(19) United States
(12) Patent Application Publication
(10) Pub. No.: US 2026/0362966 A1
(43) Pub. Date: Nov. 12, 2026

(54) ORGANIC ELECTROLUMINESCENT DEVICE AND ORGANIC ELECTROLUMINESCENT MATERIAL

(71) Applicant: DuPont Specialty Materials Korea Ltd., Chungcheongnam-do (KR)

(72) Inventors: Hyun-Woo KANG, Gyeonggi-do (KR); Kyoung-Jin PARK, Gyeonggi-do (KR); Tae-Jin LEE, Gyeonggi-do (KR); Sung-Woo JANG, Gyeonggi-do (KR); Hong-Se OH, Gyeonggi-do (KR); Hyo-Jung LEE, Gyeonggi-do (KR); Mi-Ran SEO, Gyeonggi-do (KR)

(21) Appl. No.: 19/640,360

(22) Filed: Apr. 6, 2026

(30) Foreign Application Priority Data
Apr. 16, 2025 (KR) 10-2025-0049525
Mar. 19, 2026 (KR) 10-2026-0049896

(51) Int. Cl. H10K 85/60; C07C 211/61; C07D 209/88; C07D 307/91; C07D 333/76; C07D 345/00; C07D 405/14; C07D 407/12; C07D 407/14; C07D 409/14; C07F 7/08; C07F 9/46; H10K 50/15

(52) U.S. Cl. CPC H10K 85/636; C07C 211/61; C07D 209/88; C07D 307/91; C07D 333/76; C07D 345/00; C07D 405/14; C07D 407/12; C07D 407/14; C07D 409/14; C07F 7/0816; C07F 9/46; H10K 85/633; C07C 2603/18; H10K 50/156; H10K 85/626; H10K 85/6574

(57) ABSTRACT

The present disclosure relates to an organic electroluminescent device comprising a compound represented by Formula 1 in a hole injection layer and an organic electroluminescent material for a hole injection layer, thereby providing an organic electroluminescent device having a lower driving voltage, higher luminous efficiency, higher current efficiency and/or improved lifetime characteristics compared to the conventional device.

### TECHNICAL FIELD

The present disclosure relates to an organic electroluminescent device and an organic electroluminescent material.

### BACKGROUND ART

The TPD/Alq3 bilayer small-molecule organic electroluminescent device (OLED) with green emission, which is constituted with a light-emitting layer and a charge transport layer, was first developed by Tang et al. of Eastman Kodak in 1987. Thereafter, studies on organic electroluminescent devices have proceeded rapidly, and OLEDs have since been commercialized. An OLED having a low driving voltage, high luminous efficiency, and/or long lifespan characteristics is required for long-term use and high display resolution.

In general, in single stack or double stack OLED devices used in displays, there is a problem in that lateral leakage current (LLC) occurs. The lateral leakage current is a phenomenon in which holes or electrons move in a lateral direction rather than in a vertical direction within the device, thereby causing an undesired current to flow. Such LLC is mainly caused by the influence of the p-doping concentration, and is pointed out as one of the main causes of deterioration in device performance. In addition, p-dopant materials are often monopolized by patents, so only a few companies can engage in development using them, and the difficulty of synthesis also results in high production costs. Due to these issues, there has been a continuous need for technologies capable of ensuring the performance of the hole injection layer in structures that minimize or eliminate the use of p-dopants. Accordingly, the development of a hole injection layer in a p-dopant-free structure, which can simultaneously address the LLC issue and reduce development costs, has emerged as a technically important challenge.

Korean Patent Application Laid-Open No. 10-2023-0174704 discloses a plurality of host materials and an organic electroluminescent device comprising the same, but does not specifically disclose the particular organic electroluminescent device and organic electroluminescent material for a hole injection layer as claimed in the present disclosure. In addition, there is a continuous need to develop an organic electroluminescent device having improved performance compared to a conventional device.

### DISCLOSURE OF INVENTION

### Technical Problem

The objective of the present disclosure is, first, to provide an organic electroluminescent device in which the turn-on voltage of the device is rapidly improved and lateral leakage current (LLC) does not occur, and second, to provide an organic electroluminescent material for use as a p-dopant-free hole injection layer material which is comprised in such an organic electroluminescent device.

### Solution to Problem

As a result of intensive studies to solve the technical problems, the present inventors found that the above objective can be achieved by (1) an organic electroluminescent device comprising a first electrode; a second electrode facing the first electrode; and an organic layer disposed between the first electrode and the second electrode, wherein the organic layer comprises a hole transport region, a light-emitting layer, and an electron transport region, wherein the hole transport region comprises a hole injection layer and a plurality of hole transport layers, wherein the hole injection layer comprises a compound represented by the following Formula 1, wherein each of the plurality of hole transport layers comprises at least one compound, and wherein at least two of the plurality of hole transport layers comprise different compounds from each other, (2) an organic electroluminescent material comprising a compound represented by the following Formula 1, for use in a hole injection layer, or (3) an organic electroluminescent device comprising the organic electroluminescent material.

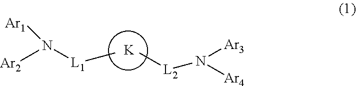

in Formula 1,
ring K is selected from the following Formulas 1-1 to 1-4;

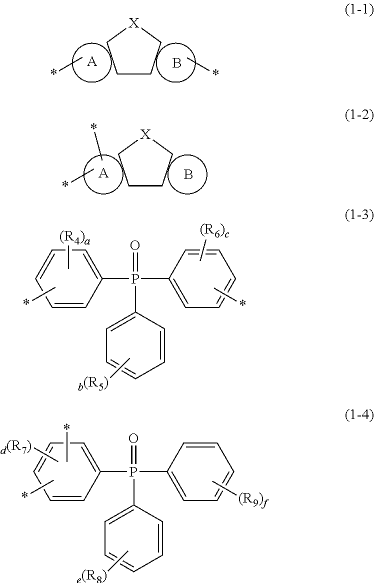

X represents —N(R₁)—, —O—, —S—, —C(R₂)(R₃)—, —Se—, —Si(R₂)(R₃)—, or —P(═O)—;
ring A and ring B each independently represent a substituted or unsubstituted (C₆-C₃₀)arene, or a substituted or unsubstituted (3- to 30-membered)heteroarene; R₁ to R₃ each independently represent a substituted or unsubstituted (C₆-C₃₀)aryl, a substituted or unsubstituted (C₁-C₃₀)alkyl, a substituted or unsubstituted (C₃-C₃₀)cycloalkyl, or a substituted or unsubstituted (3- to 30-membered)heteroaryl, or may be linked to an adjacent substituent(s) to form a substituted or unsubstituted (C₃-C₃₀) mono- or polycyclic, alicyclic, aromatic ring, or a combination thereof, wherein the carbon atoms of the formed ring may be replaced with at least one heteroatom selected from nitrogen, oxygen, and sulfur;
R₄ to R₉ and Ar₁ to Ar₄ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀)aliphatic ring(s) and a (C₆-C₃₀)aromatic ring(s), a substituted or unsubstituted mono- or di(C₁-C₃₀)alkylamino, a substituted or unsubstituted mono- or di(C₂-C₃₀)alkenylamino, a substituted or unsubstituted mono- or di(C₆-C₃₀)arylamino, a substituted or unsubstituted mono- or di(3- to 30-membered)heteroarylamino, a substituted or unsubstituted (C₁-C₃₀)alkyl(C₂-C₃₀)alkenylamino, a substituted or unsubstituted (C₁-C₃₀)alkyl(C₆-C₃₀)arylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, or a substituted or unsubstituted (C₆-C₃₀)aryl(3- to 30-membered)heteroarylamino, or may be linked to an adjacent substituent(s) to form a substituted or unsubstituted (C₃-C₃₀)mono- or polycyclic, alicyclic, aromatic ring, or a combination thereof, wherein the carbon atoms of the formed ring may be replaced with at least one heteroatom selected from nitrogen, oxygen, and sulfur;
L₁ and L₂ each independently represent a single bond, a substituted or unsubstituted (C₆-C₃₀)arylene, or a substituted or unsubstituted (3- to 30-membered)heteroarylene;
a and c each represent an integer of 1 to 4, b, e, and f each represent an integer of 1 to 5, and d represents an integer of 1 to 3; and
the heteroaryl(ene) comprises at least one heteroatom selected from B, N, O, S, Si, Se, and P.

### Advantageous Effects of Invention

An organic electroluminescent material according to the present disclosure exhibits performance suitable for use in organic electroluminescent devices. In addition, the organic electroluminescent device of the present disclosure may reduce the turn-on voltage and/or lateral leakage current (LLC). Further, an organic electroluminescent device exhibiting a lower driving voltage, higher current efficiency, higher luminous efficiency, and/or improved lifetime characteristics compared to the conventional device can be provided, regardless of the presence or absence of a p-dopant. Finally, a display device or a lighting device using the same can be manufactured.

### MODE FOR INVENTION

Hereinafter, the present disclosure will be described in detail. However, the following description is intended to explain the present disclosure, and is not meant in any way to restrict the scope of the present disclosure.

The “organic electroluminescent material” in the present disclosure is a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a charge-generating material, an N-type charge-generating material, a P-type charge-generating material, a light-emitting auxiliary material, an electron-blocking material, a light-emitting material (including a host material and a dopant material), an electron buffer material, a hole-blocking material, an electron transport material, an electron injection material, etc.

Herein, the term “(C₁-C₃₀)alkyl” is meant to refer to a linear or branched alkyl having 1 to 30 carbon atoms constituting a chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. The term “(C₃-C₃₀)cycloalkyl” is meant to refer to a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, norbornyl, adamantyl, etc. The term “(3- to 7-membered)heterocycloalkyl” in the present disclosure is meant to refer to a saturated or partially unsaturated monocyclic or polycyclic ring-shaped hydrocarbon substituent having 3 to 7, preferably 5 to 7 ring backbone atoms, and including at least one heteroatom selected from the group consisting of B, N, O, S, Si, P, and Se. The above heterocycloalkyl may include tetrahydrofuran, pyrrolidine, thiolane, tetrahydropyran, etc.

The “(C₆-C₃₀)aryl”, “(C₆-C₃₀)arylene”, or “(C₆-C₃₀)arene” in the present disclosure refers to a monocyclic or fused ring radical derived from an aromatic hydrocarbon having 6 to 30 ring backbone carbon atoms, and may be partially saturated. The number of the ring backbone carbon atoms is preferably 6 to 20, more preferably 6 to 15. The above aryl may comprise a spiro structure. The above aryl may include phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluorene-fluorene]yl, spiro[fluorene-benzofluorene]yl, azulenyl, tetramethyl-dihydrophenanthrenyl, etc. Specifically, the aryl may include o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The “(3- to 30-membered)heteroaryl”, “(3- to 30-membered)heteroarylene”, or “(3- to 30-membered)heteroarene” in the present disclosure refers to an aryl group, arylene group, or arene group having 3 to 30 ring backbone atoms and including at least one heteroatom(s) selected from the group consisting of B, N, O, S, Si, P, and Se. Herein, the number of ring backbone atoms is preferably 3 to 30, and more preferably 5 to 20. The number of heteroatoms is preferably 1 to 4. The above heteroaryl or heteroarylene may be a monocyclic ring or a fused ring condensed with at least one benzene ring, and may be partially saturated. In addition, the above heteroaryl or heteroarylene may be one formed by linking at least one heteroaryl or aryl group to a heteroaryl group via a single bond(s), and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzofuroquinolinyl, benzofuroquinazolinyl, benzofuronaphthyridinyl, benzofuropyrimidinyl, naphthofuropyrimidinyl, benzothienoquinoline, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzisothiazolyl, benzisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolyl, thiochromenoquinazolyl, dimethylbenzopyrimidinyl, indolocarbazolyl, indenocarbazolyl, etc. More specifically, the heteroaryl may include 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Additionally, “heteroaryl(ene)” can be classified into a heteroaryl(ene) with electronic properties and a heteroaryl(ene) with hole properties. A heteroaryl(ene) with electronic properties is a substituent that is relatively rich in electrons in the parent nucleus, for example, a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted quinolyl, etc. A heteroaryl(ene) with hole properties is a substituent that is relatively electron-deficient in the parent nucleus, for example, a substituted or unsubstituted carbazolyl, a substituted or unsubstituted dibenzofuranyl, a substituted or unsubstituted dibenzothiophenyl, etc.

Herein, “a fused ring group of a (C₃-C₃₀)aliphatic ring(s) and a (C₆-C₃₀)aromatic ring(s)” is meant to be a functional group of a ring in which at least one aliphatic ring having 3 to 30 ring backbone carbon atoms preferably 3 to 25 ring backbone carbon atoms, and more preferably 5 to 18 ring backbone carbon atoms, is fused with at least one aromatic ring having 6 to 30 ring backbone carbon atoms, preferably 6 to 25 ring backbone carbon atoms, and more preferably 6 to 18 ring backbone carbon atoms. Specific examples of the fused ring group include a fused ring group of one or more benzene and one or more cyclohexane, a fused ring group of one or more naphthalene and one or more cyclopentane, or a fused ring group of fluorene and cyclohexane, etc. Herein, the carbon atom of the fused ring group of a (C₃-C₃₀)aliphatic ring(s) and a (C₆-C₃₀)aromatic ring(s) may be replaced with one or more heteroatoms selected from B, N, O, S, Si, P, and Se. Herein, “halogen” includes F, Cl, Br, and I.

In addition, “ortho-” (“o-”), “meta-” (“m-”), and “para” (“p-”) are prefixes which each represent the relative positions of substituents. The prefix “ortho-” indicates that two substituents are adjacent to each other, and for example, when two substituents in a benzene derivative occupy positions 1 and 2, this is called an “ortho-” configuration. The prefix “meta-” indicates that two substituents are at positions 1 and 3, and for example, when two substituents in a benzene derivative occupy positions 1 and 3, this is called a “meta-” configuration. The prefix “para-” indicates that two substituents are at positions 1 and 4, and for example, when two substituents in a benzene derivative occupy positions 1 and 4, this is called a “para-” configuration.

Herein, “substituted” in the expression “substituted or unsubstituted” means that a hydrogen atom in a certain functional group is replaced with another atom or another functional group, i.e., a substituent, and this also includes substitution by a group in which two or more of the substituents are connected. Unless otherwise specified, the substituent may replace hydrogen at a position where the substituent can be substituted without limitation, and when two or more hydrogen atoms in a certain functional group are each replaced with a substituent, each substituent may be the same as or different from each other. The maximum number of substituents that can be substituted for a certain functional group may be the total number of valences that can be substituted for each atom forming the functional group. Herein, the substituted alkyl, the substituted aryl, the substituted arylene, the substituted arene, the substituted heteroaryl, the substituted heteroarylene, the substituted heteroarene, the substituted cycloalkyl, the substituted alkoxy, the substituted trialkylsilyl, the substituted dialkylarylsilyl, the substituted alkyldiarylsilyl, the substituted triarylsilyl, and the substituted fused ring group of an aliphatic ring(s) and an aromatic ring(s), the substituted mono- or dialkylamino, the substituted mono- or dialkenylamino, the substituted mono- or diarylamino, the substituted mono- or diheteroarylamino, the substituted alkylalkenylamino, the substituted alkylarylamino, the substituted alkylheteroarylamino, the substituted alkenylarylamino, the substituted alkenylheteroarylamino, the substituted arylheteroarylamino, the substituted mono- or polycyclic, alicyclic, aromatic ring, or a combination thereof each independently may be substituted with at least one selected from the group consisting of deuterium, a halogen, a cyano, a carboxyl, a nitro, a hydroxy, phosphinoxide, a (C₁-C₃₀)alkyl, a halo(C₁-C₃₀)alkyl, a (C₂-C₃₀)alkenyl, a (C₂-C₃₀)alkynyl, a (C₁-C₃₀)alkoxy, a (C₁-C₃₀)alkylthio, a (C₃-C₃₀)cycloalkyl, a (C₃-C₃₀)cycloalkenyl, a (3- to 7-membered)heterocycloalkyl, a (C₆-C₃₀)aryloxy, a (C₆-C₃₀)arylthio, a (3- to 30-membered)heteroaryl, a (C₆-C₃₀)aryl, a tri(C₁-C₃₀)alkylsilyl, a tri(C₆-C₃₀)arylsilyl, a di(C₁-C₃₀)alkyl(C₆-C₃₀)arylsilyl, a (C₁-C₃₀)alkyldi(C₆-C₃₀)arylsilyl, a fused ring group of a (C₃-C₃₀)aliphatic ring(s) and a (C₆-C₃₀)aromatic ring(s), an amino, a mono- or di(C₁-C₃₀)alkylamino, a mono- or di(C₂-C₃₀)alkenylamino, (C₁-C₃₀)alkyl(C₂-C₃₀)alkenylamino, a mono- or di(C₆-C₃₀)arylamino, a (C₁-C₃₀)alkyl(C₆-C₃₀)arylamino, a mono- or di(3- to 30-membered)heteroarylamino, a (C₁-C₃₀)alkyl(3- to 30-membered)heteroarylamino, a (C₂-C₃₀)alkenyl(C₆-C₃₀)arylamino, a (C₂-C₃₀)alkenyl(3- to 30-membered)heteroarylamino, a (C₆-C₃₀)aryl(3- to 30-membered)heteroarylamino, a (C₁-C₃₀)alkylcarbonyl, a (C₁-C₃₀)alkoxycarbonyl, a (C₆-C₃₀)arylcarbonyl, a (C₆-C₃₀)arylphosphinyl, a di(C₆-C₃₀)arylboronyl, a di(C₁-C₃₀)alkylboronyl, a (C₁-C₃₀)alkyl(C₆-C₃₀)arylboronyl, a (C₆-C₃₀)aryl(C₁-C₃₀)alkyl, a (C₁-C₃₀)alkyl(C₆-C₃₀)aryl, and combinations thereof. According to one embodiment of the present disclosure, the group is deuterium, a (C₁-C₂₀)alkyl, a (C₃-C₁₈)cycloalkyl, a (C₆-C₂₅)aryl, a (3- to 25-membered)heteroaryl, and combinations thereof. According to another embodiment of the present disclosure, the group is deuterium, a (C₁-C₆)alkyl, a (C₆-C₁₀)cycloalkyl, a (C₆-C₁₈)aryl, and combinations thereof. For example, the group may be deuterium, a methyl, a cyclohexyl, a norbornyl, an adamantyl, a naphthyl, etc., and these may be further substituted with deuterium.

In the present disclosure, if a substituent is not indicated in the chemical formula or compound structure, it may mean that all possible positions for the substituent are hydrogen or deuterium. That is, in the case of deuterium, it is an isotope of hydrogen, and some hydrogen atoms may be the isotope deuterium, and in this case, the content of deuterium may be 0% to 100%. In the present disclosure, in cases where a substituent is not indicated in the chemical formula or compound structure, if the substituent does not explicitly exclude deuterium, such as 0% deuterium, 100% hydrogen, and all substituents are hydrogen, hydrogen and deuterium may be used intermixed in a compound. Deuterium is one of the isotopes of hydrogen and an element with a deuteron consisting of one proton and one neutron as its nucleus. It can be represented as hydrogen-2, whose element symbol can also be written as D or ²H. Isotopes are atoms with the same atomic number (Z) but different mass numbers (A), and can also be interpreted as elements with the same number of protons but different numbers of neutrons.

In the present disclosure, “a combination thereof” refers to a combination of one or more elements from the corresponding list to form a known or chemically stable arrangement that can be envisioned by a person skilled in the art from the corresponding list. For example, alkyl and deuterium can be combined to form a partially or fully deuterated alkyl group, a halogen and alkyl can be combined to form a halogenated alkyl substituent, and a halogen, alkyl, and aryl can be combined to form a halogenated arylalkyl. For example, a preferred combination of substituents includes up to 50 atoms that are not hydrogen or deuterium, or up to 40 atoms that are not hydrogen or deuterium, or up to 30 atoms that are not hydrogen or deuterium, or in many cases, a preferred combination of substituents may comprise up to 20 atoms that are not hydrogen or deuterium.

In the formulas of the present disclosure, when there are multiple substituents represented by the same symbol, each substituent represented by the same symbol may be the same as or different from each other.

The compound represented by Formula 1 is described in more detail as follows.

In Formula 1, L₁ and L₂ each independently represent a single bond, a substituted or unsubstituted (C₆-C₃₀)arylene, or a substituted or unsubstituted (3- to 30-membered)heteroarylene. According to one embodiment of the present disclosure, L₁ and L₂ each independently represent a single bond, or a substituted or unsubstituted (C₆-C₂₅)arylene. According to another embodiment of the present disclosure, L₁ and L₂ each independently represent a single bond, or a substituted or unsubstituted (C₆-C₁₈)arylene. For example, L₁ and L₂ each independently may be a single bond, a phenylene, etc., and these may be further substituted with deuterium.

In Formula 1, Ar₁ to Ar₄ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀)aliphatic ring(s) and a (C₆-C₃₀)aromatic ring(s), a substituted or unsubstituted mono- or di(C₁-C₃₀)alkylamino, a substituted or unsubstituted mono- or di(C₂-C₃₀)alkenylamino, a substituted or unsubstituted mono- or di(C₆-C₃₀)arylamino, a substituted or unsubstituted mono- or di(3- to 30-membered)heteroarylamino, a substituted or unsubstituted (C₁-C₃₀)alkyl(C₂-C₃₀)alkenylamino, a substituted or unsubstituted (C₁-C₃₀)alkyl(C₆-C₃₀)arylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, or a substituted or unsubstituted (C₆-C₃₀)aryl(3- to 30-membered)heteroarylamino, or may be linked to an adjacent substituent(s) to form a substituted or unsubstituted (C₃-C₃₀)mono- or polycyclic, alicyclic, aromatic ring, or a combination thereof, wherein the carbon atoms of the formed ring may be replaced with at least one heteroatom selected from nitrogen, oxygen, and sulfur. Ar₁ to Ar₄ may each be the same as or different from one another. According to one embodiment of the present disclosure, Ar₁ to Ar₄ each independently represent a substituted or unsubstituted (C₆-C₂₅)aryl, a substituted or unsubstituted (3- to 25-membered)heteroaryl, a substituted or unsubstituted (C₆-C₁₈)cycloalkyl, or a substituted or unsubstituted fused ring group of a (C₃-C₁₀)aliphatic ring(s) and a (C₆-C₂₅)aromatic ring(s). According to another embodiment of the present disclosure, Ar₁ to Ar₄ each independently represent a substituted or unsubstituted (C₆-C₁₈)aryl, a substituted or unsubstituted (13- to 17-membered)heteroaryl, or a substituted or unsubstituted fused ring group of a (C₃-C₁₀)aliphatic ring(s) and a (C₆-C₁₈)aromatic ring(s), and these may be substituted with at least one selected from the group consisting of a (C₁-C₃₀)alkyl, a (C₆-C₃₀)cycloalkyl, and a (C₆-C₃₀)aryl. For example, Ar₁ to Ar₄ each independently may be a phenyl unsubstituted or substituted with a methyl, a naphthyl, a norbornyl, an adamantyl, or a cyclohexyl, a naphthyl, a naphthylphenyl, a phenylnaphthyl, a biphenyl, a terphenyl, a dimethylfluorenyl, a dimethylbenzofluorenyl, a dibenzofuranyl, a dibenzothiophenyl, a benzonaphthofuranyl, a fused ring group of a cyclohexyl and a dimethylflourenyl, etc., and these may be further substituted with deuterium.

In Formulas 1-1 to 1-4, ring A and ring B each independently represent a substituted or unsubstituted (C₆-C₃₀)arene, or a substituted or unsubstituted (3- to 30-membered)heteroarene. Ring A and ring B may each be the same as or different from each other. According to one embodiment of the present disclosure, ring A and ring B each independently represent a substituted or unsubstituted (C₆-C₁₈)arene. According to another embodiment of the present disclosure, ring A and ring B each independently represent a substituted or unsubstituted (C₆-C₁₄)arene. For example, ring A and ring B each independently may be a benzene ring, a naphthalene ring, a phenanthrene ring, etc.

According to one embodiment of the present disclosure, the ring A and the ring B may each independently be selected from the following Formulas 1-5 to 1-9.

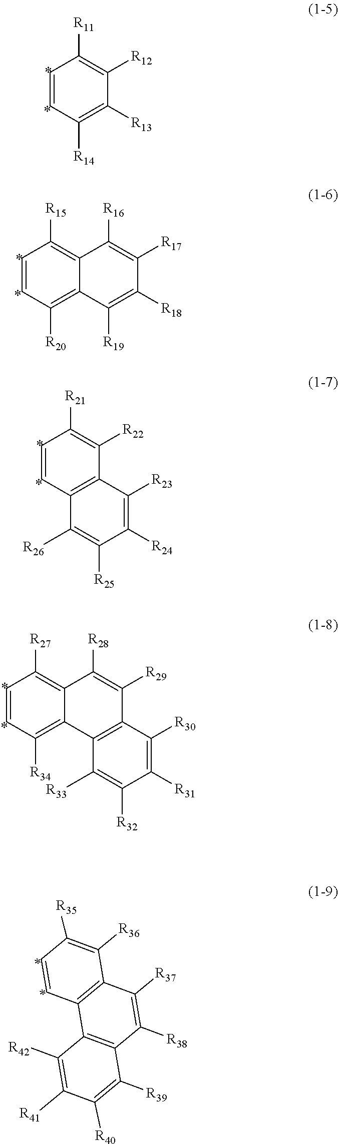

In Formulas 1-5 to 1-9, R₁₁ to R₄₂ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀)aliphatic ring(s) and a (C₆-C₃₀)aromatic ring(s), a substituted or unsubstituted mono- or di(C₁-C₃₀)alkylamino, a substituted or unsubstituted mono- or di(C₂-C₃₀)alkenylamino, a substituted or unsubstituted mono- or di(C₆-C₃₀)arylamino, a substituted or unsubstituted mono- or di(3- to 30-membered)heteroarylamino, a substituted or unsubstituted (C₁-C₃₀)alkyl(C₂-C₃₀)alkenylamino, a substituted or unsubstituted (C₁-C₃₀)alkyl(C₆-C₃₀)arylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, or a substituted or unsubstituted (C₆-C₃₀)aryl(3- to 30-membered)heteroarylamino, or may be linked to an adjacent substituent(s) to form a substituted or unsubstituted (C₃-C₃₀) mono- or polycyclic, alicyclic, aromatic ring, or a combination thereof, wherein the carbon atoms of the formed ring may be replaced with at least one heteroatom selected from nitrogen, oxygen, and sulfur. For example, R₁₁ to R₄₂ each independently may be hydrogen, or deuterium.

In Formulas 1-1 to 1-4, X represents —N(R₁)—, —O—, —S—, —C(R₂)(R₃)—, —Se—, —Si(R₂)(R₃)—, or —P(═O)—. According to one embodiment of the present disclosure, X represents —N(R₁)—, —O—, —S—, —C(R₂)(R₃)—, —Se—, or —Si(R₂)(R₃)—.

R₁ to R₃ each independently represent a substituted or unsubstituted (C₆-C₃₀)aryl, a substituted or unsubstituted (C₁-C₃₀)alkyl, a substituted or unsubstituted (C₃-C₃₀)cycloalkyl, or a substituted or unsubstituted (3- to 30-membered)heteroaryl, or may be linked to an adjacent substituent(s) to form a substituted or unsubstituted (C₃-C₃₀) mono- or polycyclic, alicyclic, aromatic ring, or a combination thereof, wherein the carbon atoms of the formed ring may be replaced with at least one heteroatom selected from nitrogen, oxygen, and sulfur. According to one embodiment of the present disclosure, R₁ to R₃ each independently represent a substituted or unsubstituted (C₁-C₁₀)alkyl, or a substituted or unsubstituted (C₆-C₂₅)aryl. According to another embodiment of the present disclosure, R₁ represents a substituted or unsubstituted (C₆-C₁₈)aryl, and R₂ and R₃ each independently represent a substituted or unsubstituted (C₁-C₆)alkyl, or a substituted or unsubstituted (C₆-C₁₈)aryl. For example, R₁ may be a phenyl, a naphthyl, etc., and R₂ and R₃ each independently may be a methyl, an ethyl, a phenyl, etc., and these may be further substituted with deuterium.

In Formulas 1-3 and 1-4, R₄ to R₉ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀)aliphatic ring(s) and a (C₆-C₃₀)aromatic ring(s), a substituted or unsubstituted mono- or di(C₁-C₃₀)alkylamino, a substituted or unsubstituted mono- or di(C₂-C₃₀)alkenylamino, a substituted or unsubstituted mono- or di(C₆-C₃₀)arylamino, a substituted or unsubstituted mono- or di(3- to 30-membered)heteroarylamino, a substituted or unsubstituted (C₁-C₃₀)alkyl(C₂-C₃₀)alkenylamino, a substituted or unsubstituted (C₁-C₃₀)alkyl(C₆-C₃₀)arylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, or a substituted or unsubstituted (C₆-C₃₀)aryl(3- to 30-membered)heteroarylamino, or may be linked to an adjacent substituent(s) to form a substituted or unsubstituted (C₃-C₃₀) mono- or polycyclic, alicyclic, aromatic ring, or a combination thereof, wherein the carbon atoms of the formed ring may be replaced with at least one heteroatom selected from nitrogen, oxygen, and sulfur. For example, R₄ to R₉ each independently may be hydrogen, or deuterium.

In Formulas 1-3 and 1-4, a and c each represent an integer of 1 to 4, b, e, and f each represent an integer of 1 to 5, and d represents an integer of 1 to 3. If a to f are each an integer of 2 or greater, R₄ to R₉ may each be the same as or different from one another.

The organic electroluminescent compound represented by Formula 1 may be at least one selected from the following compounds, but is not limited thereto.

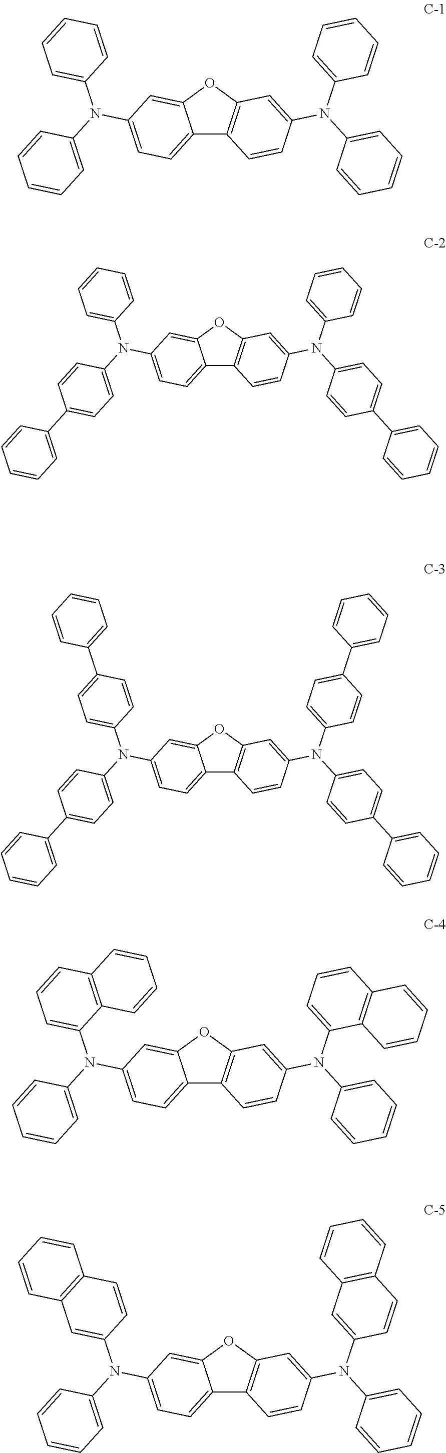 [SMILES: c1ccc(N(c2ccccc2)c2ccc3c(c2)oc2cc(N(c4ccccc4)c4ccccc4)ccc23)cc1 ; c1ccc(-c2ccc(N(c3ccccc3)c3ccc4c(c3)oc3cc(N(c5ccccc5)c5ccc(-c6ccccc6)cc5)ccc34)cc2)cc1 ; c1ccc(-c2ccc(N(c3ccc(-c4ccccc4)cc3)c3ccc4c(c3)oc3cc(N(c5ccc(-c6ccccc6)cc5)c5ccc(-c6ccccc6)cc5)ccc34)cc2)cc1 ; c1ccc(N(c2ccc3c(c2)oc2cc(N(c4ccccc4)c4cccc5ccccc45)ccc23)c2cccc3ccccc23)cc1 ; c1ccc(N(c2ccc3ccccc3c2)c2ccc3c(c2)oc2cc(N(c4ccccc4)c4ccc5ccccc5c4)ccc23)cc1]

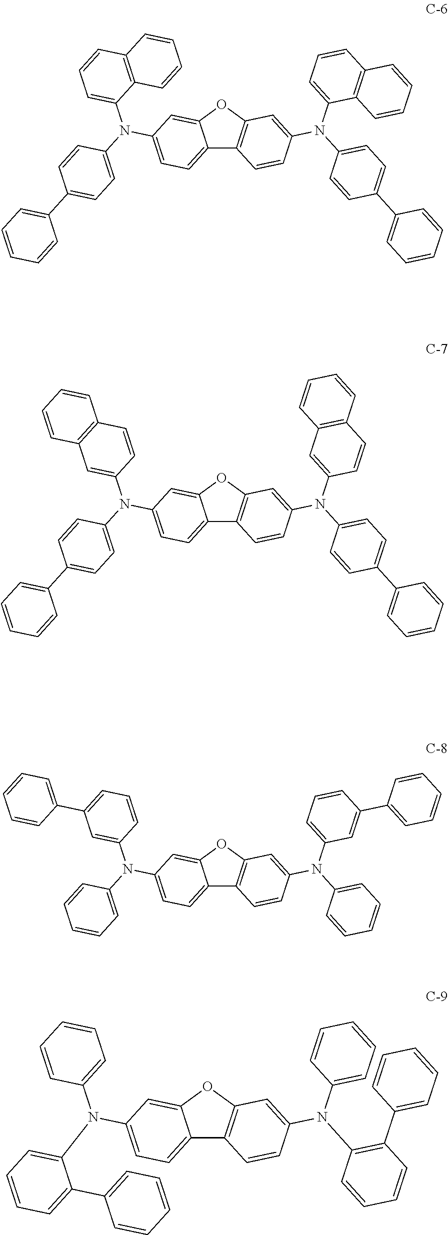 [SMILES: c1ccc(-c2ccc(N(c3ccc4c(c3)oc3cc(N(c5ccc(-c6ccccc6)cc5)c5cccc6ccccc56)ccc34)c3cccc4ccccc34)cc2)cc1 ; c1ccc(-c2ccc(N(c3ccc4ccccc4c3)c3ccc4c(c3)oc3cc(N(c5ccc(-c6ccccc6)cc5)c5ccc6ccccc6c5)ccc34)cc2)cc1 ; c1ccc(-c2cccc(N(c3ccccc3)c3ccc4c(c3)oc3cc(N(c5ccccc5)c5cccc(-c6ccccc6)c5)ccc34)c2)cc1 ; c1ccc(-c2ccccc2N(c2ccccc2)c2ccc3c(c2)oc2cc(N(c4ccccc4)c4ccccc4-c4ccccc4)ccc23)cc1]

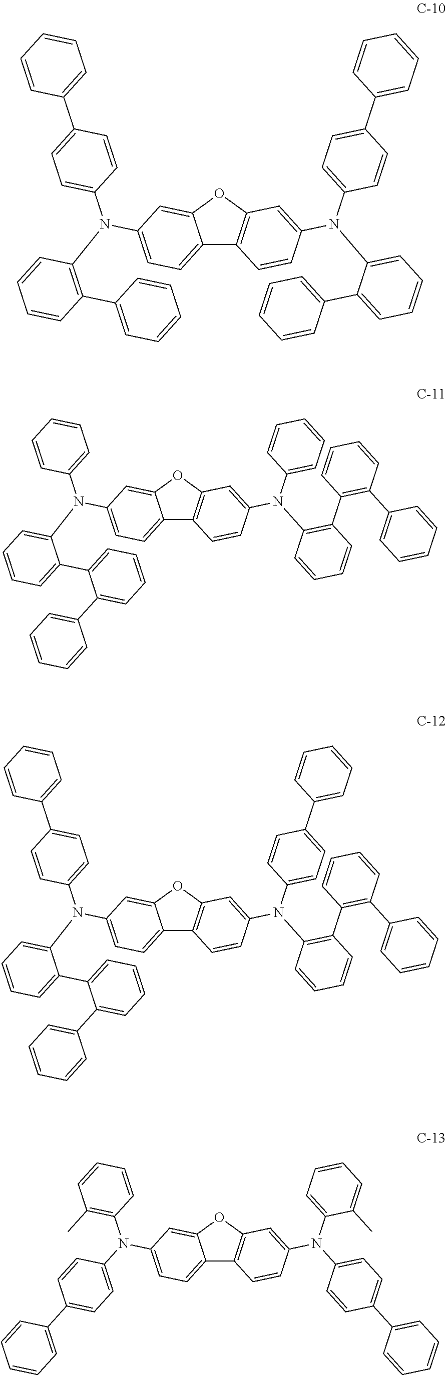 [SMILES: c1ccc(-c2ccc(N(c3ccc4c(c3)oc3cc(N(c5ccc(-c6ccccc6)cc5)c5ccccc5-c5ccccc5)ccc34)c3ccccc3-c3ccccc3)cc2)cc1 ; c1ccc(-c2ccccc2-c2ccccc2N(c2ccccc2)c2ccc3c(c2)oc2cc(N(c4ccccc4)c4ccccc4-c4ccccc4-c4ccccc4)ccc23)cc1 ; c1ccc(-c2ccc(N(c3ccc4c(c3)oc3cc(N(c5ccc(-c6ccccc6)cc5)c5ccccc5-c5ccccc5-c5ccccc5)ccc34)c3ccccc3-c3ccccc3-c3ccccc3)cc2)cc1 ; Cc1ccccc1N(c1ccc(-c2ccccc2)cc1)c1ccc2c(c1)oc1cc(N(c3ccc(-c4ccccc4)cc3)c3ccccc3C)ccc12]

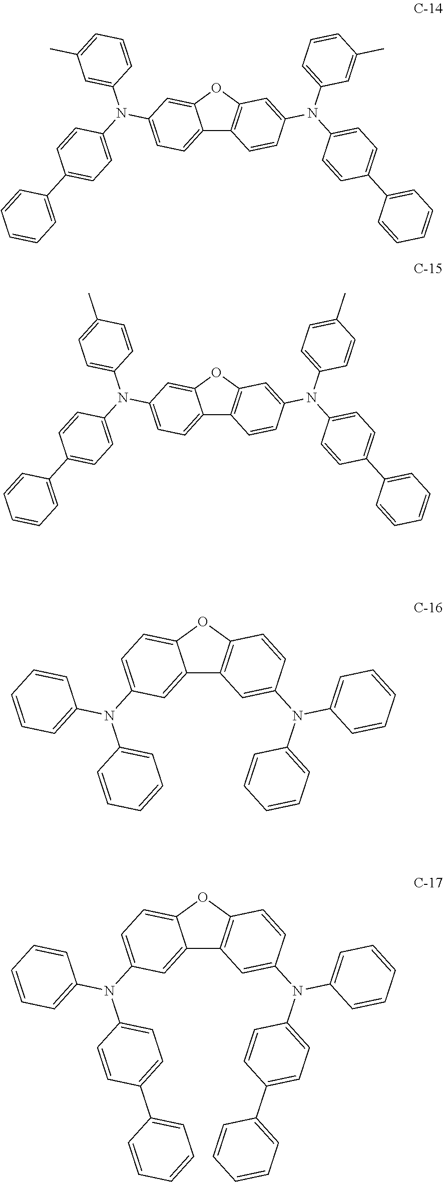 [SMILES: Cc1cccc(N(c2ccc(-c3ccccc3)cc2)c2ccc3c(c2)oc2cc(N(c4ccc(-c5ccccc5)cc4)c4cccc(C)c4)ccc23)c1 ; Cc1ccc(N(c2ccc(-c3ccccc3)cc2)c2ccc3c(c2)oc2cc(N(c4ccc(C)cc4)c4ccc(-c5ccccc5)cc4)ccc23)cc1 ; c1ccc(N(c2ccccc2)c2ccc3oc4ccc(N(c5ccccc5)c5ccccc5)cc4c3c2)cc1 ; c1ccc(-c2ccc(N(c3ccccc3)c3ccc4oc5ccc(N(c6ccccc6)c6ccc(-c7ccccc7)cc6)cc5c4c3)cc2)cc1]

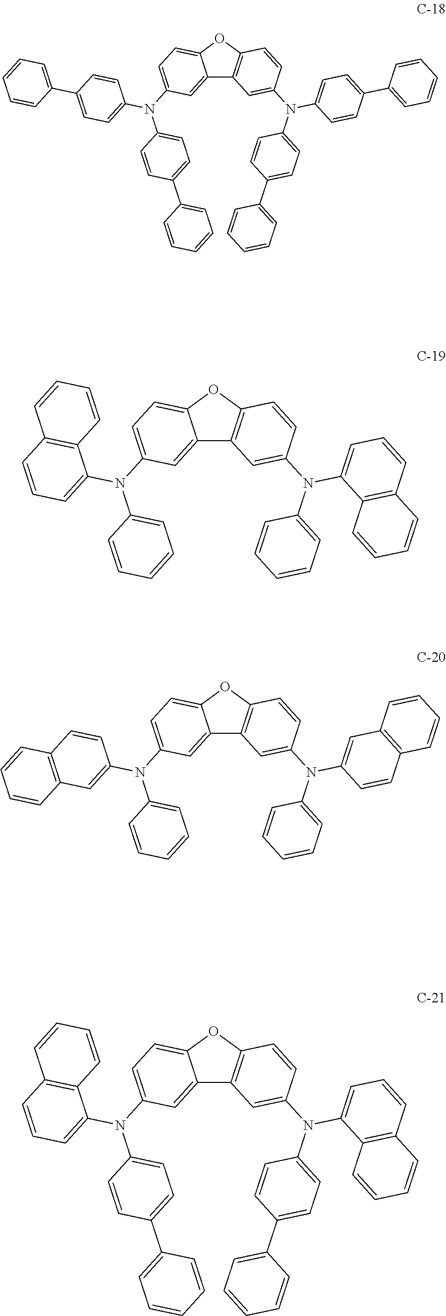 [SMILES: c1ccc(-c2ccc(N(c3ccc(-c4ccccc4)cc3)c3ccc4oc5ccc(N(c6ccc(-c7ccccc7)cc6)c6ccc(-c7ccccc7)cc6)cc5c4c3)cc2)cc1 ; c1ccc(N(c2ccc3oc4ccc(N(c5ccccc5)c5cccc6ccccc56)cc4c3c2)c2cccc3ccccc23)cc1 ; c1ccc(N(c2ccc3ccccc3c2)c2ccc3oc4ccc(N(c5ccccc5)c5ccc6ccccc6c5)cc4c3c2)cc1 ; c1ccc(-c2ccc(N(c3ccc4oc5ccc(N(c6ccc(-c7ccccc7)cc6)c6cccc7ccccc67)cc5c4c3)c3cccc4ccccc34)cc2)cc1]

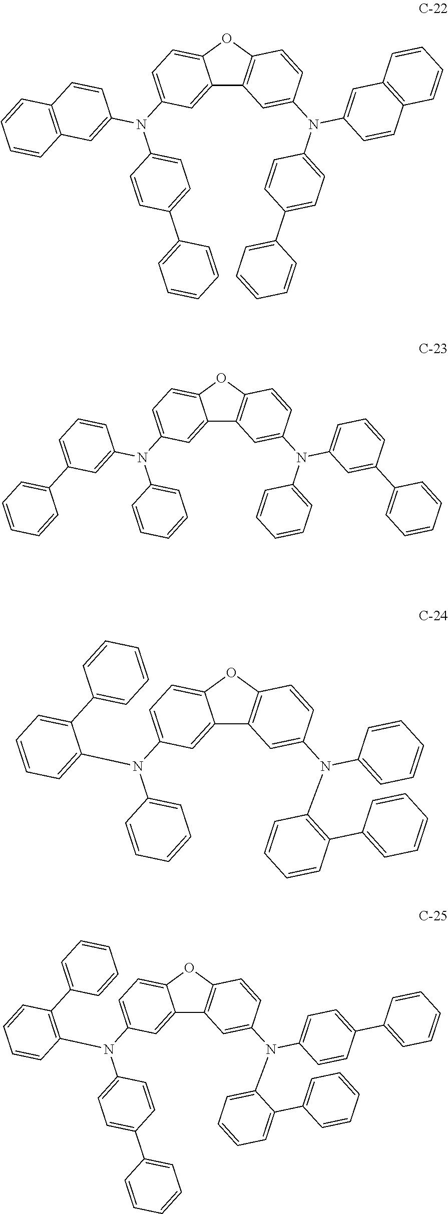 [SMILES: c1ccc(-c2ccc(N(c3ccc4ccccc4c3)c3ccc4oc5ccc(N(c6ccc(-c7ccccc7)cc6)c6ccc7ccccc7c6)cc5c4c3)cc2)cc1 ; c1ccc(-c2cccc(N(c3ccccc3)c3ccc4oc5ccc(N(c6ccccc6)c6cccc(-c7ccccc7)c6)cc5c4c3)c2)cc1 ; c1ccc(-c2ccccc2N(c2ccccc2)c2ccc3oc4ccc(N(c5ccccc5)c5ccccc5-c5ccccc5)cc4c3c2)cc1 ; c1ccc(-c2ccc(N(c3ccc4oc5ccc(N(c6ccc(-c7ccccc7)cc6)c6ccccc6-c6ccccc6)cc5c4c3)c3ccccc3-c3ccccc3)cc2)cc1]

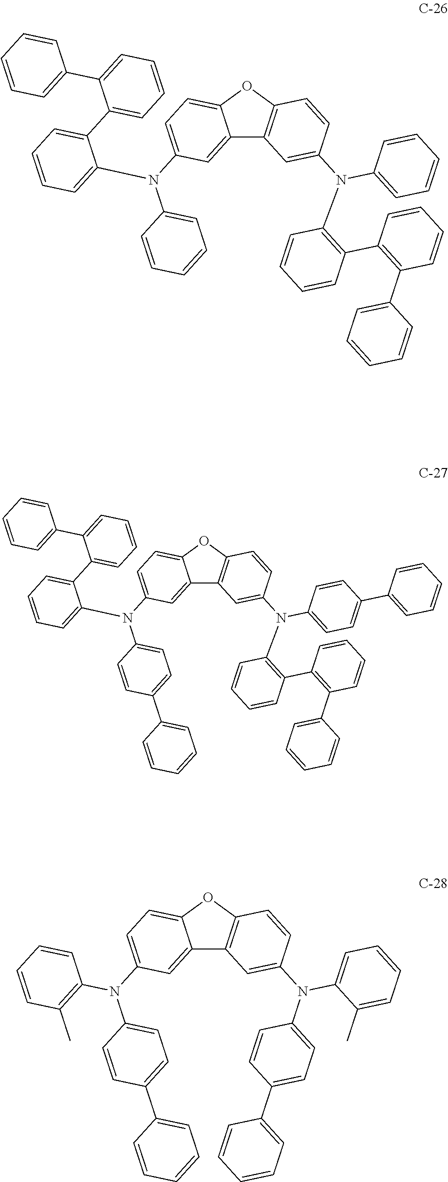 [SMILES: c1ccc(-c2ccccc2-c2ccccc2N(c2ccccc2)c2ccc3oc4ccc(N(c5ccccc5)c5ccccc5-c5ccccc5-c5ccccc5)cc4c3c2)cc1 ; c1ccc(-c2ccc(N(c3ccc4oc5ccc(N(c6ccc(-c7ccccc7)cc6)c6ccccc6-c6ccccc6-c6ccccc6)cc5c4c3)c3ccccc3-c3ccccc3-c3ccccc3)cc2)cc1 ; Cc1ccccc1N(c1ccc(-c2ccccc2)cc1)c1ccc2oc3ccc(N(c4ccc(-c5ccccc5)cc4)c4ccccc4C)cc3c2c1]

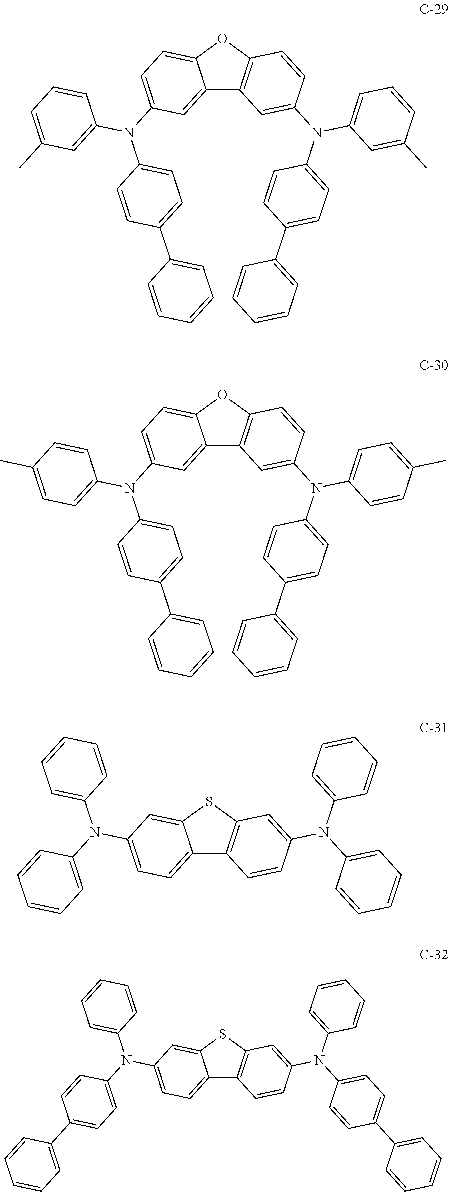

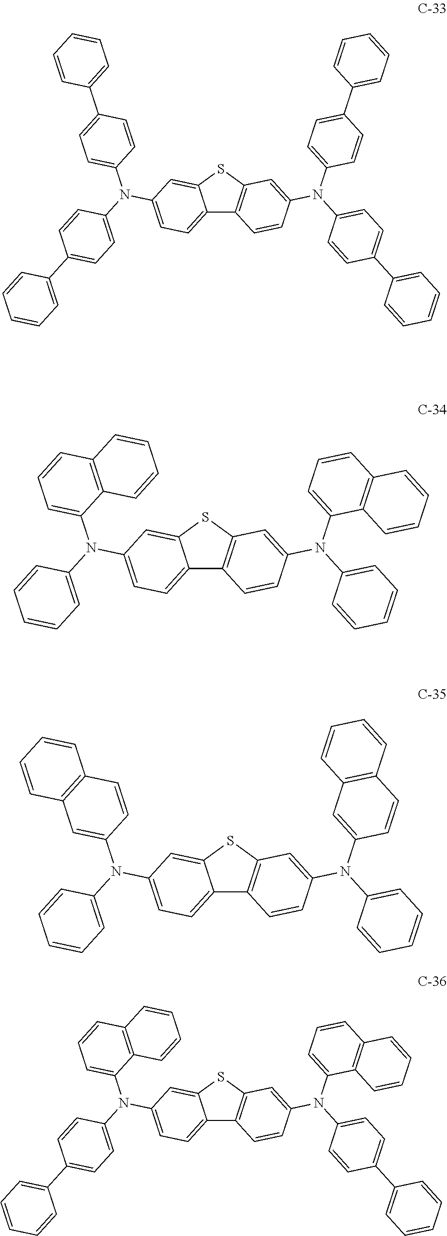 [SMILES: c1ccc(-c2ccc(N(c3ccc(-c4ccccc4)cc3)c3ccc4c(c3)sc3cc(N(c5ccc(-c6ccccc6)cc5)c5ccc(-c6ccccc6)cc5)ccc34)cc2)cc1 ; c1ccc(N(c2ccc3c(c2)sc2cc(N(c4ccccc4)c4cccc5ccccc45)ccc23)c2cccc3ccccc23)cc1 ; c1ccc(N(c2ccc3ccccc3c2)c2ccc3c(c2)sc2cc(N(c4ccccc4)c4ccc5ccccc5c4)ccc23)cc1 ; c1ccc(-c2ccc(N(c3ccc4c(c3)sc3cc(N(c5ccc(-c6ccccc6)cc5)c5cccc6ccccc56)ccc34)c3cccc4ccccc34)cc2)cc1]

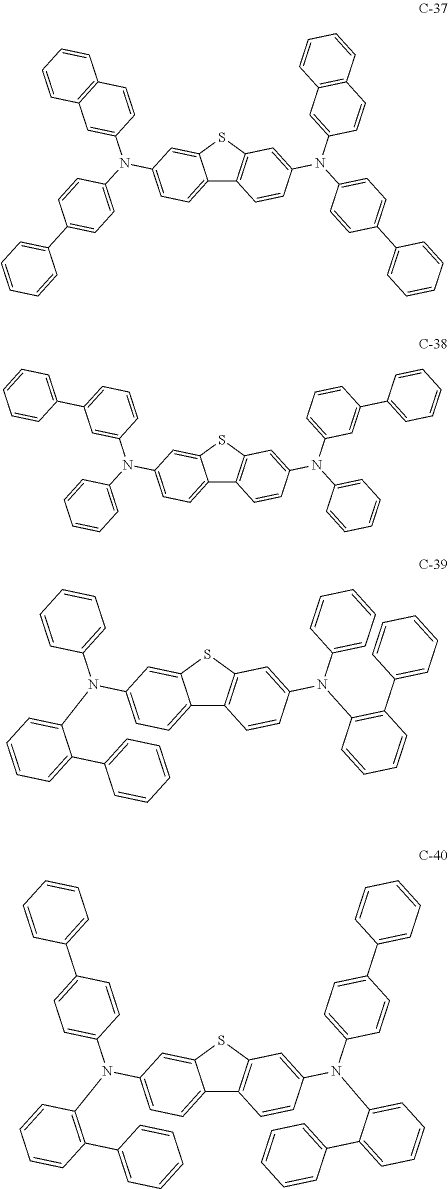 [SMILES: c1ccc(-c2ccc(N(c3ccc4ccccc4c3)c3ccc4c(c3)sc3cc(N(c5ccc(-c6ccccc6)cc5)c5ccc6ccccc6c5)ccc34)cc2)cc1 ; c1ccc(-c2cccc(N(c3ccccc3)c3ccc4c(c3)sc3cc(N(c5ccccc5)c5cccc(-c6ccccc6)c5)ccc34)c2)cc1 ; c1ccc(-c2ccccc2N(c2ccccc2)c2ccc3c(c2)sc2cc(N(c4ccccc4)c4ccccc4-c4ccccc4)ccc23)cc1 ; c1ccc(-c2ccc(N(c3ccc4c(c3)sc3cc(N(c5ccc(-c6ccccc6)cc5)c5ccccc5-c5ccccc5)ccc34)c3ccccc3-c3ccccc3)cc2)cc1]

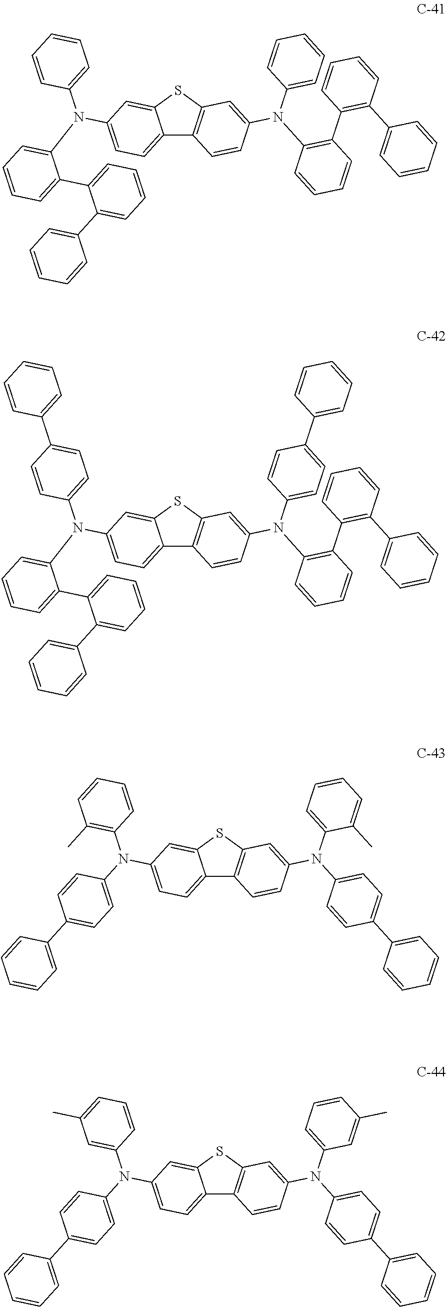 [SMILES: c1ccc(-c2ccccc2-c2ccccc2N(c2ccccc2)c2ccc3c(c2)sc2cc(N(c4ccccc4)c4ccccc4-c4ccccc4-c4ccccc4)ccc23)cc1 ; c1ccc(-c2ccc(N(c3ccc4c(c3)sc3cc(N(c5ccc(-c6ccccc6)cc5)c5ccccc5-c5ccccc5-c5ccccc5)ccc34)c3ccccc3-c3ccccc3-c3ccccc3)cc2)cc1 ; Cc1ccccc1N(c1ccc(-c2ccccc2)cc1)c1ccc2c(c1)sc1cc(N(c3ccc(-c4ccccc4)cc3)c3ccccc3C)ccc12 ; Cc1cccc(N(c2ccc(-c3ccccc3)cc2)c2ccc3c(c2)sc2cc(N(c4ccc(-c5ccccc5)cc4)c4cccc(C)c4)ccc23)c1]

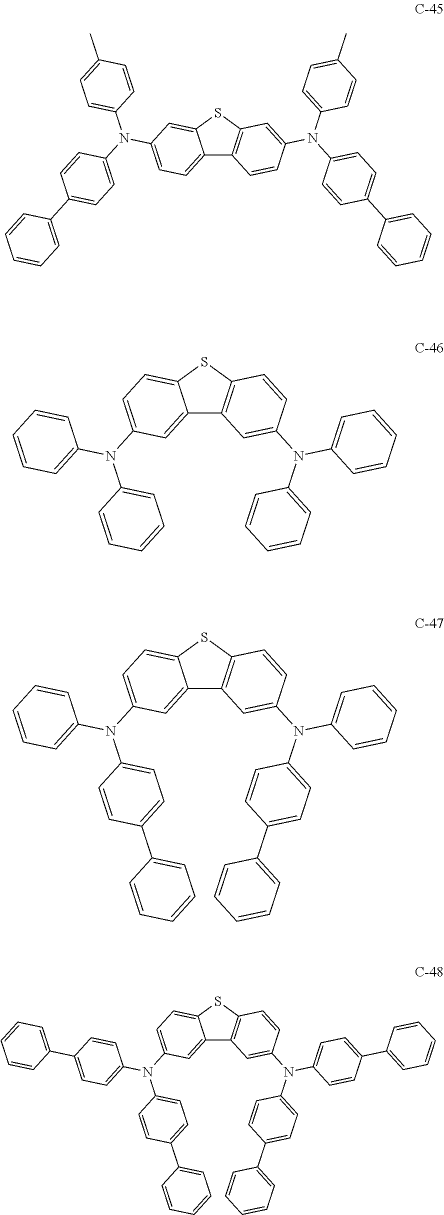 [SMILES: Cc1ccc(N(c2ccc(-c3ccccc3)cc2)c2ccc3c(c2)sc2cc(N(c4ccc(C)cc4)c4ccc(-c5ccccc5)cc4)ccc23)cc1 ; c1ccc(N(c2ccccc2)c2ccc3sc4ccc(N(c5ccccc5)c5ccccc5)cc4c3c2)cc1 ; c1ccc(-c2ccc(N(c3ccccc3)c3ccc4sc5ccc(N(c6ccccc6)c6ccc(-c7ccccc7)cc6)cc5c4c3)cc2)cc1 ; c1ccc(-c2ccc(N(c3ccc(-c4ccccc4)cc3)c3ccc4sc5ccc(N(c6ccc(-c7ccccc7)cc6)c6ccc(-c7ccccc7)cc6)cc5c4c3)cc2)cc1]

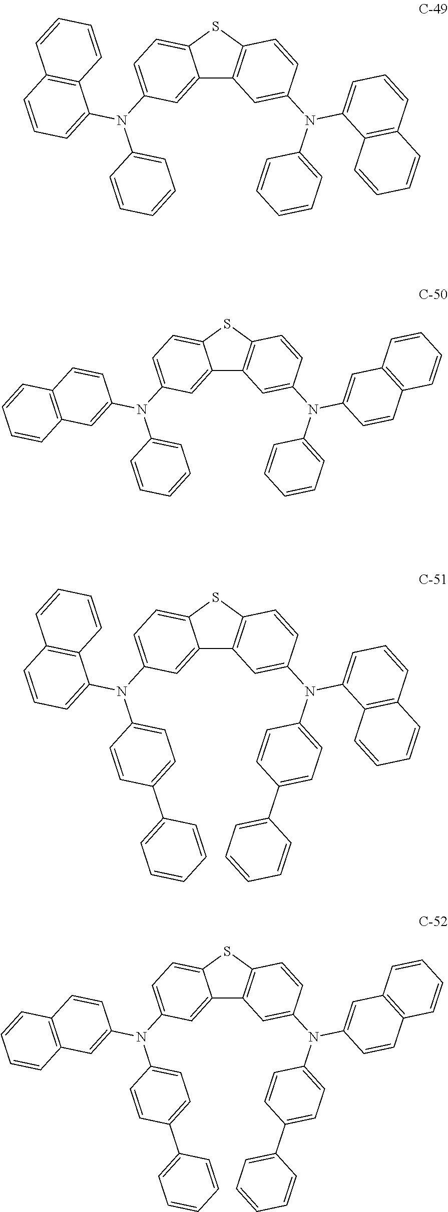 [SMILES: c1ccc(N(c2ccc3sc4ccc(N(c5ccccc5)c5cccc6ccccc56)cc4c3c2)c2cccc3ccccc23)cc1 ; c1ccc(N(c2ccc3ccccc3c2)c2ccc3sc4ccc(N(c5ccccc5)c5ccc6ccccc6c5)cc4c3c2)cc1 ; c1ccc(-c2ccc(N(c3ccc4sc5ccc(N(c6ccc(-c7ccccc7)cc6)c6cccc7ccccc67)cc5c4c3)c3cccc4ccccc34)cc2)cc1 ; c1ccc(-c2ccc(N(c3ccc4ccccc4c3)c3ccc4sc5ccc(N(c6ccc(-c7ccccc7)cc6)c6ccc7ccccc7c6)cc5c4c3)cc2)cc1]

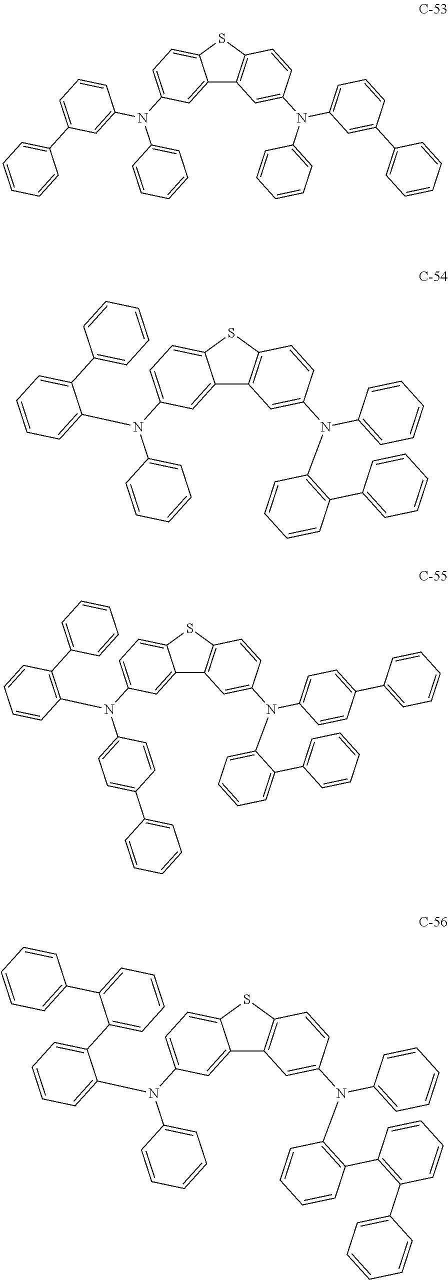 [SMILES: c1ccc(-c2cccc(N(c3ccccc3)c3ccc4sc5ccc(N(c6ccccc6)c6cccc(-c7ccccc7)c6)cc5c4c3)c2)cc1 ; c1ccc(-c2ccccc2N(c2ccccc2)c2ccc3sc4ccc(N(c5ccccc5)c5ccccc5-c5ccccc5)cc4c3c2)cc1 ; c1ccc(-c2ccc(N(c3ccc4sc5ccc(N(c6ccc(-c7ccccc7)cc6)c6ccccc6-c6ccccc6)cc5c4c3)c3ccccc3-c3ccccc3)cc2)cc1 ; c1ccc(-c2ccccc2-c2ccccc2N(c2ccccc2)c2ccc3sc4ccc(N(c5ccccc5)c5ccccc5-c5ccccc5-c5ccccc5)cc4c3c2)cc1]

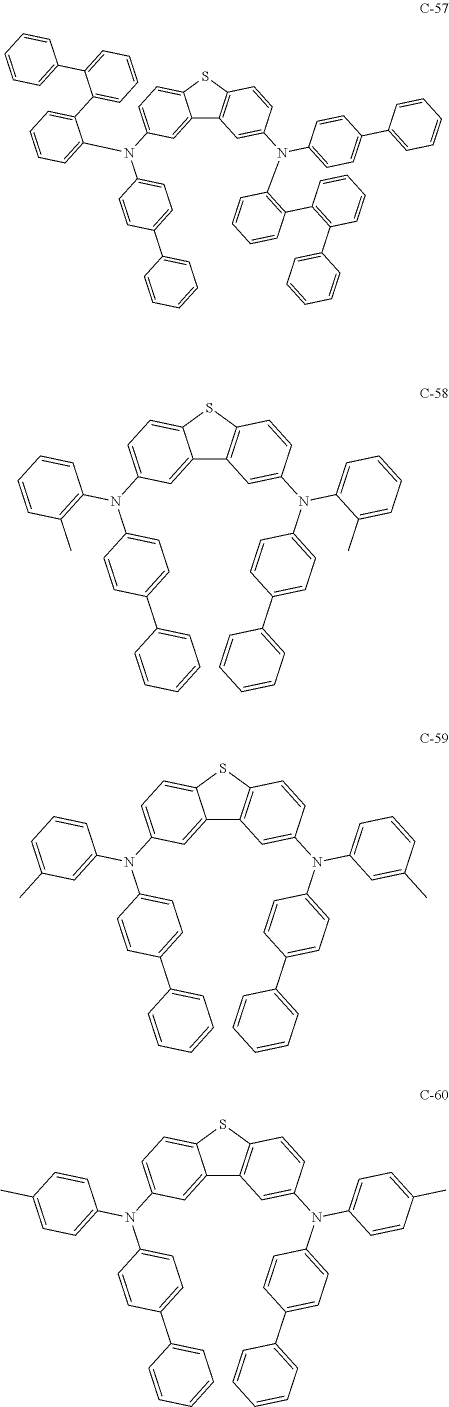

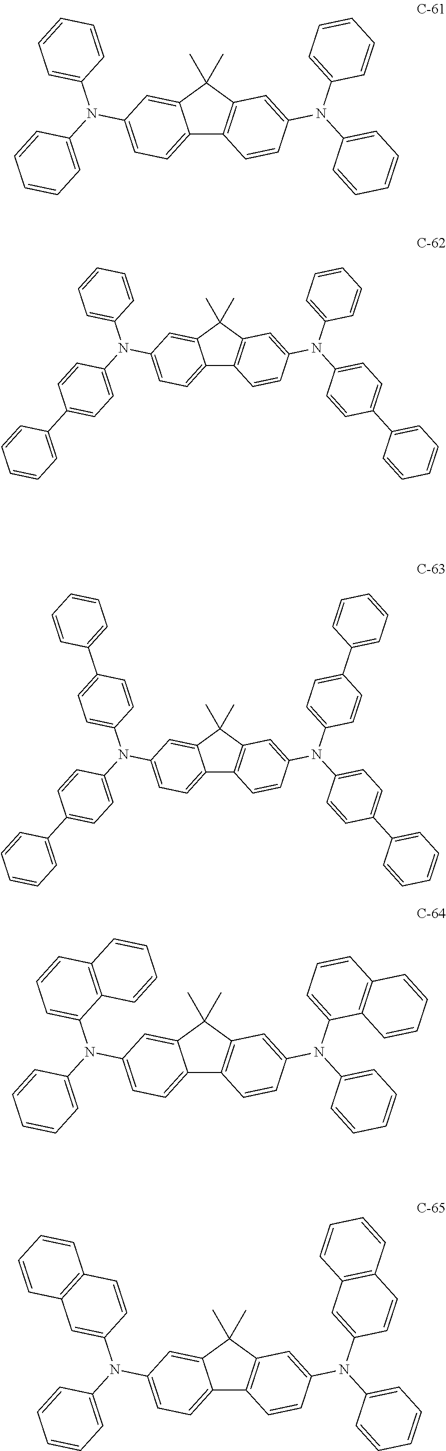 [SMILES: CC1(C)c2cc(N(c3ccccc3)c3ccccc3)ccc2-c2ccc(N(c3ccccc3)c3ccccc3)cc21 ; CC1(C)c2cc(N(c3ccccc3)c3ccc(-c4ccccc4)cc3)ccc2-c2ccc(N(c3ccccc3)c3ccc(-c4ccccc4)cc3)cc21 ; CC1(C)c2cc(N(c3ccc(-c4ccccc4)cc3)c3ccc(-c4ccccc4)cc3)ccc2-c2ccc(N(c3ccc(-c4ccccc4)cc3)c3ccc(-c4ccccc4)cc3)cc21 ; CC1(C)c2cc(N(c3ccccc3)c3cccc4ccccc34)ccc2-c2ccc(N(c3ccccc3)c3cccc4ccccc34)cc21 ; CC1(C)c2cc(N(c3ccccc3)c3ccc4ccccc4c3)ccc2-c2ccc(N(c3ccccc3)c3ccc4ccccc4c3)cc21]

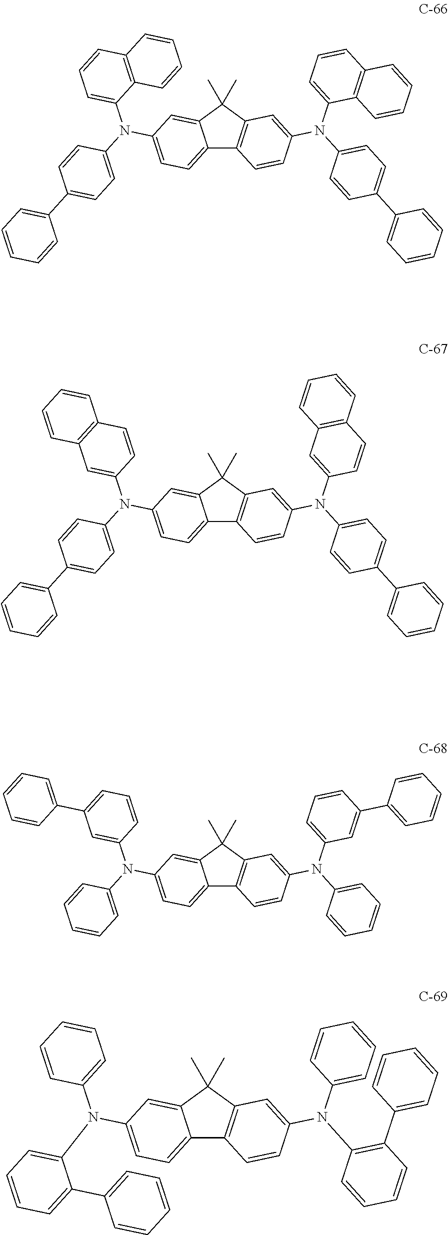 [SMILES: CC1(C)c2cc(N(c3ccc(-c4ccccc4)cc3)c3cccc4ccccc34)ccc2-c2ccc(N(c3ccc(-c4ccccc4)cc3)c3cccc4ccccc34)cc21 ; CC1(C)c2cc(N(c3ccc(-c4ccccc4)cc3)c3ccc4ccccc4c3)ccc2-c2ccc(N(c3ccc(-c4ccccc4)cc3)c3ccc4ccccc4c3)cc21 ; CC1(C)c2cc(N(c3ccccc3)c3cccc(-c4ccccc4)c3)ccc2-c2ccc(N(c3ccccc3)c3cccc(-c4ccccc4)c3)cc21 ; CC1(C)c2cc(N(c3ccccc3)c3ccccc3-c3ccccc3)ccc2-c2ccc(N(c3ccccc3)c3ccccc3-c3ccccc3)cc21]

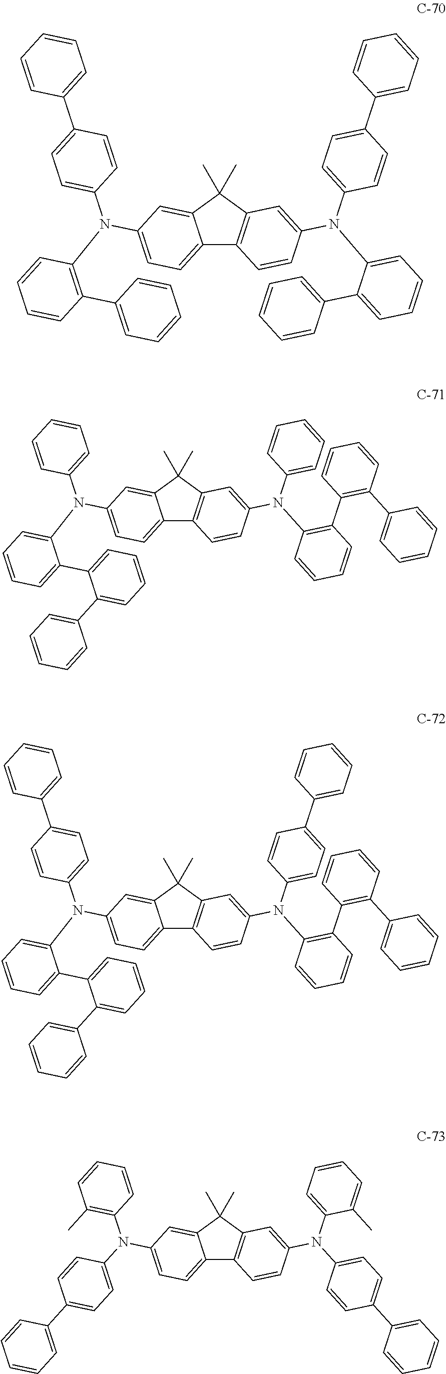 [SMILES: CC1(C)c2cc(N(c3ccc(-c4ccccc4)cc3)c3ccccc3-c3ccccc3)ccc2-c2ccc(N(c3ccc(-c4ccccc4)cc3)c3ccccc3-c3ccccc3)cc21 ; CC1(C)c2cc(N(c3ccccc3)c3ccccc3-c3ccccc3-c3ccccc3)ccc2-c2ccc(N(c3ccccc3)c3ccccc3-c3ccccc3-c3ccccc3)cc21 ; CC1(C)c2cc(N(c3ccc(-c4ccccc4)cc3)c3ccccc3-c3ccccc3-c3ccccc3)ccc2-c2ccc(N(c3ccc(-c4ccccc4)cc3)c3ccccc3-c3ccccc3-c3ccccc3)cc21 ; Cc1ccccc1N(c1ccc(-c2ccccc2)cc1)c1ccc2c(c1)C(C)(C)c1cc(N(c3ccc(-c4ccccc4)cc3)c3ccccc3C)ccc1-2]

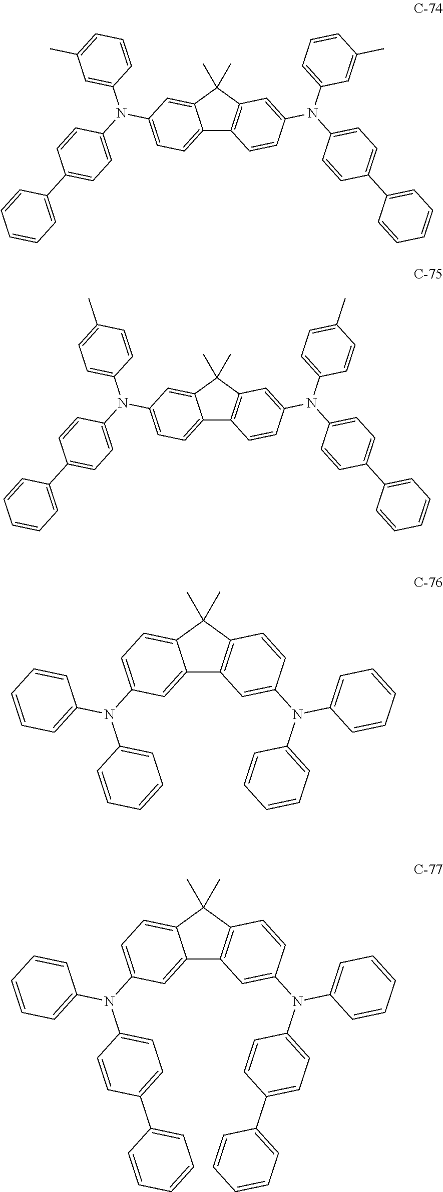 [SMILES: Cc1cccc(N(c2ccc(-c3ccccc3)cc2)c2ccc3c(c2)C(C)(C)c2cc(N(c4ccc(-c5ccccc5)cc4)c4cccc(C)c4)ccc2-3)c1 ; Cc1ccc(N(c2ccc(-c3ccccc3)cc2)c2ccc3c(c2)C(C)(C)c2cc(N(c4ccc(C)cc4)c4ccc(-c5ccccc5)cc4)ccc2-3)cc1 ; CC1(C)c2ccc(N(c3ccccc3)c3ccccc3)cc2-c2cc(N(c3ccccc3)c3ccccc3)ccc21 ; CC1(C)c2ccc(N(c3ccccc3)c3ccc(-c4ccccc4)cc3)cc2-c2cc(N(c3ccccc3)c3ccc(-c4ccccc4)cc3)ccc21]

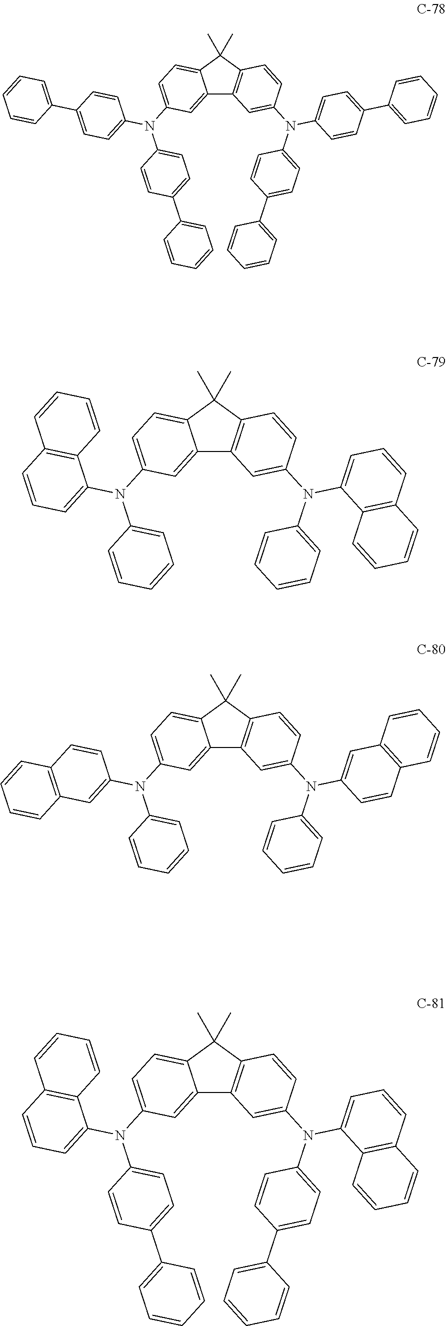 [SMILES: CC1(C)c2ccc(N(c3ccc(-c4ccccc4)cc3)c3ccc(-c4ccccc4)cc3)cc2-c2cc(N(c3ccc(-c4ccccc4)cc3)c3ccc(-c4ccccc4)cc3)ccc21 ; CC1(C)c2ccc(N(c3ccccc3)c3cccc4ccccc34)cc2-c2cc(N(c3ccccc3)c3cccc4ccccc34)ccc21 ; CC1(C)c2ccc(N(c3ccccc3)c3ccc4ccccc4c3)cc2-c2cc(N(c3ccccc3)c3ccc4ccccc4c3)ccc21 ; CC1(C)c2ccc(N(c3ccc(-c4ccccc4)cc3)c3cccc4ccccc34)cc2-c2cc(N(c3ccc(-c4ccccc4)cc3)c3cccc4ccccc34)ccc21]

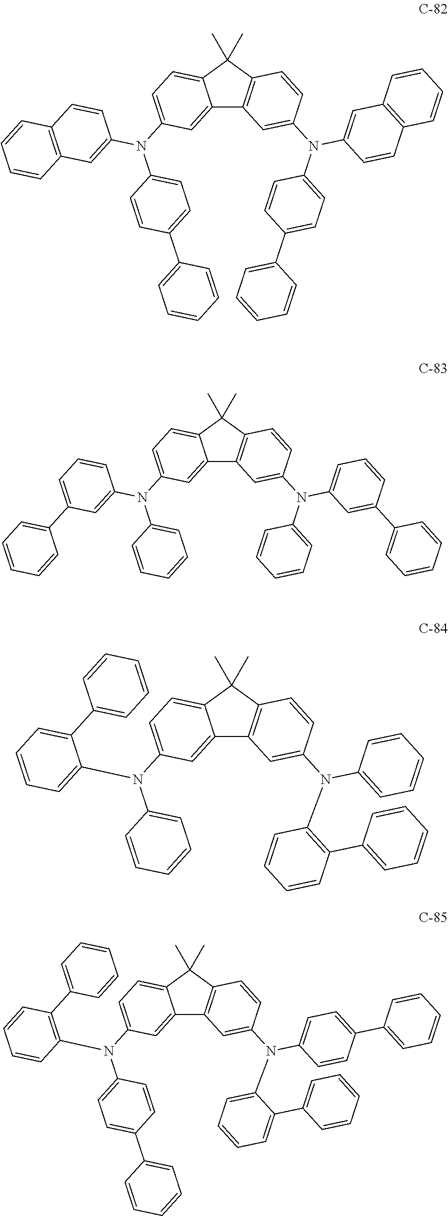 [SMILES: CC1(C)c2ccc(N(c3ccc(-c4ccccc4)cc3)c3ccc4ccccc4c3)cc2-c2cc(N(c3ccc(-c4ccccc4)cc3)c3ccc4ccccc4c3)ccc21 ; CC1(C)c2ccc(N(c3ccccc3)c3cccc(-c4ccccc4)c3)cc2-c2cc(N(c3ccccc3)c3cccc(-c4ccccc4)c3)ccc21 ; CC1(C)c2ccc(N(c3ccccc3)c3ccccc3-c3ccccc3)cc2-c2cc(N(c3ccccc3)c3ccccc3-c3ccccc3)ccc21 ; CC1(C)c2ccc(N(c3ccc(-c4ccccc4)cc3)c3ccccc3-c3ccccc3)cc2-c2cc(N(c3ccc(-c4ccccc4)cc3)c3ccccc3-c3ccccc3)ccc21]

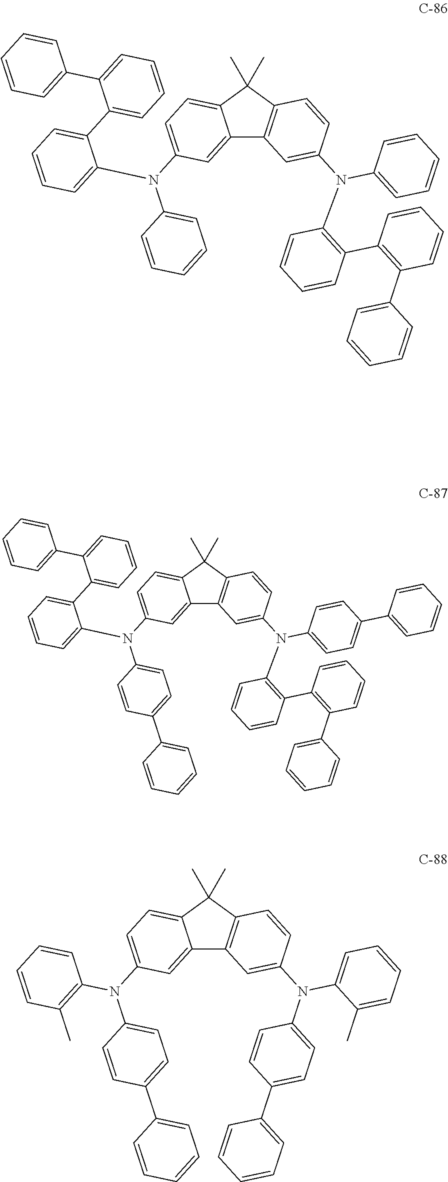 [SMILES: CC1(C)c2ccc(N(c3ccccc3)c3ccccc3-c3ccccc3-c3ccccc3)cc2-c2cc(N(c3ccccc3)c3ccccc3-c3ccccc3-c3ccccc3)ccc21 ; CC1(C)c2ccc(N(c3ccc(-c4ccccc4)cc3)c3ccccc3-c3ccccc3-c3ccccc3)cc2-c2cc(N(c3ccc(-c4ccccc4)cc3)c3ccccc3-c3ccccc3-c3ccccc3)ccc21 ; Cc1ccccc1N(c1ccc(-c2ccccc2)cc1)c1ccc2c(c1)-c1cc(N(c3ccc(-c4ccccc4)cc3)c3ccccc3C)ccc1C2(C)C]

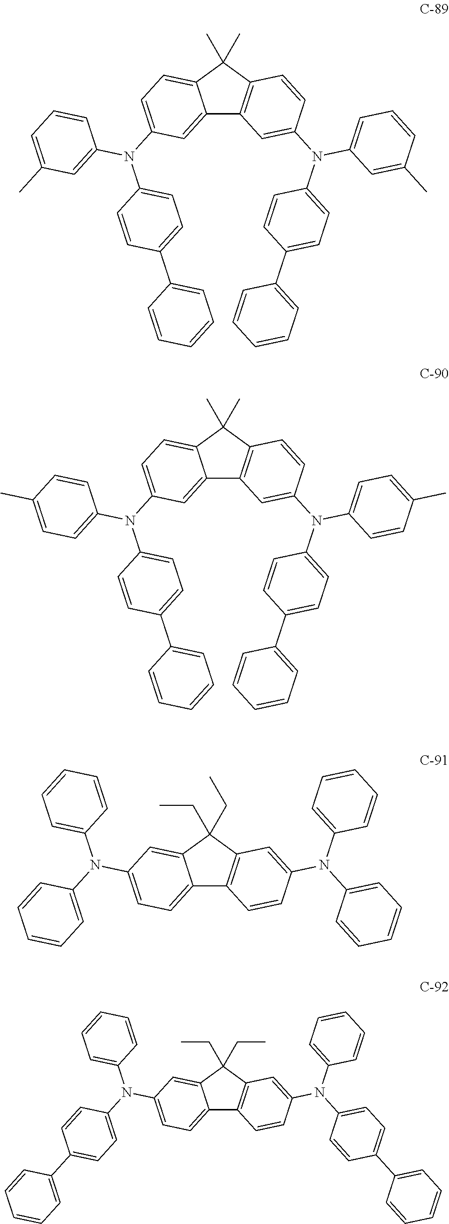

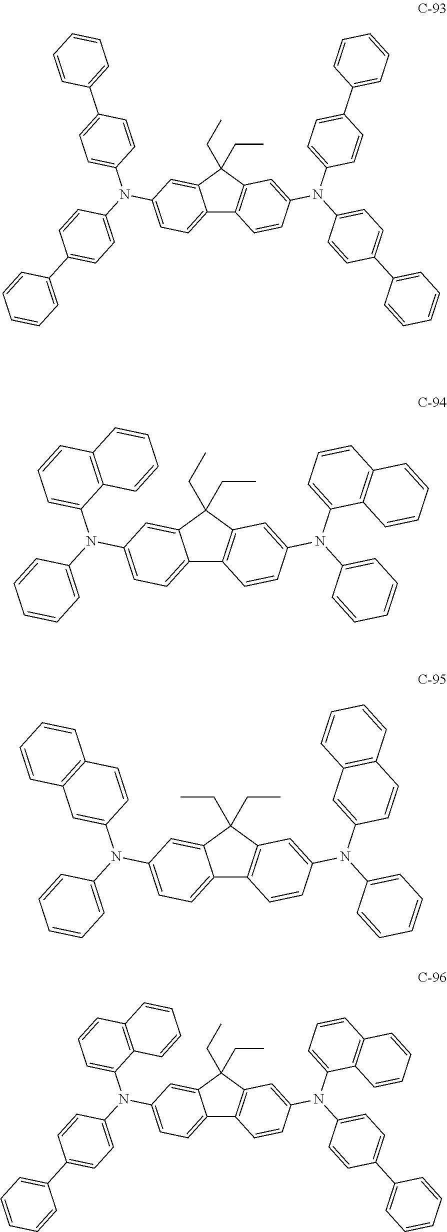 [SMILES: CCC1(CC)c2cc(N(c3ccc(-c4ccccc4)cc3)c3ccc(-c4ccccc4)cc3)ccc2-c2ccc(N(c3ccc(-c4ccccc4)cc3)c3ccc(-c4ccccc4)cc3)cc21 ; CCC1(CC)c2cc(N(c3ccccc3)c3cccc4ccccc34)ccc2-c2ccc(N(c3ccccc3)c3cccc4ccccc34)cc21 ; CCC1(CC)c2cc(N(c3ccccc3)c3ccc4ccccc4c3)ccc2-c2ccc(N(c3ccccc3)c3ccc4ccccc4c3)cc21 ; CCC1(CC)c2cc(N(c3ccc(-c4ccccc4)cc3)c3cccc4ccccc34)ccc2-c2ccc(N(c3ccc(-c4ccccc4)cc3)c3cccc4ccccc34)cc21]

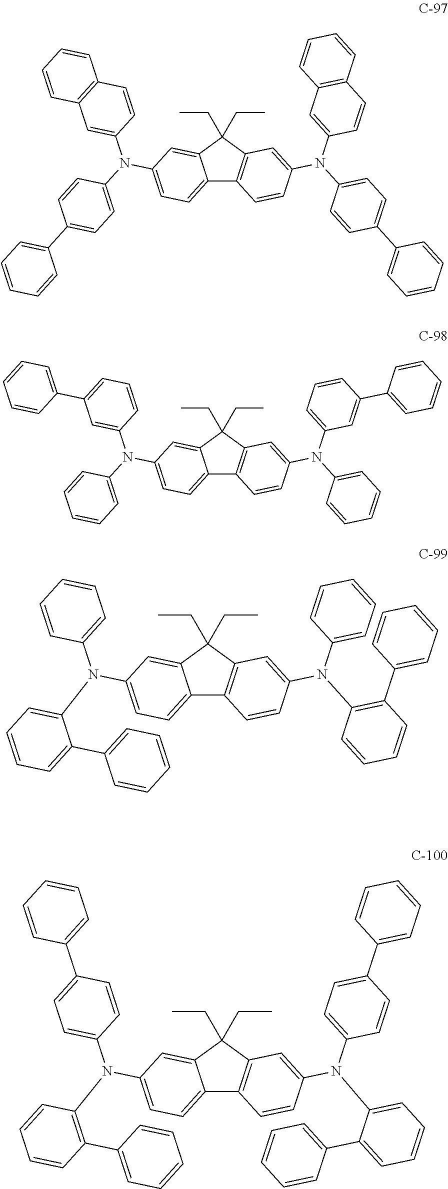 [SMILES: CCC1(CC)c2cc(N(c3ccc(-c4ccccc4)cc3)c3ccc4ccccc4c3)ccc2-c2ccc(N(c3ccc(-c4ccccc4)cc3)c3ccc4ccccc4c3)cc21 ; CCC1(CC)c2cc(N(c3ccccc3)c3cccc(-c4ccccc4)c3)ccc2-c2ccc(N(c3ccccc3)c3cccc(-c4ccccc4)c3)cc21 ; CCC1(CC)c2cc(N(c3ccccc3)c3ccccc3-c3ccccc3)ccc2-c2ccc(N(c3ccccc3)c3ccccc3-c3ccccc3)cc21 ; CCC1(CC)c2cc(N(c3ccc(-c4ccccc4)cc3)c3ccccc3-c3ccccc3)ccc2-c2ccc(N(c3ccc(-c4ccccc4)cc3)c3ccccc3-c3ccccc3)cc21]

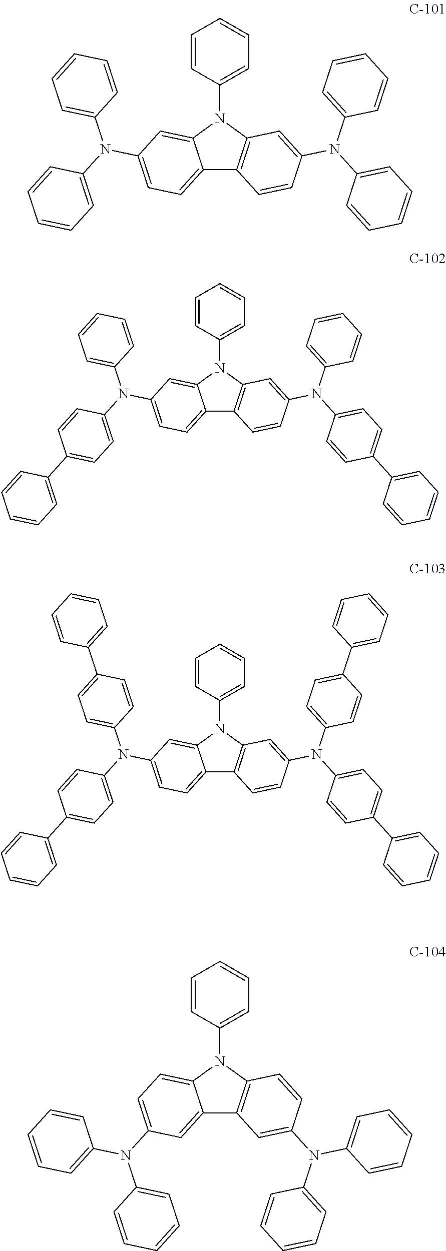 [SMILES: c1ccc(N(c2ccccc2)c2ccc3c4ccc(N(c5ccccc5)c5ccccc5)cc4n(-c4ccccc4)c3c2)cc1 ; c1ccc(-c2ccc(N(c3ccccc3)c3ccc4c5ccc(N(c6ccccc6)c6ccc(-c7ccccc7)cc6)cc5n(-c5ccccc5)c4c3)cc2)cc1 ; c1ccc(-c2ccc(N(c3ccc(-c4ccccc4)cc3)c3ccc4c5ccc(N(c6ccc(-c7ccccc7)cc6)c6ccc(-c7ccccc7)cc6)cc5n(-c5ccccc5)c4c3)cc2)cc1 ; c1ccc(N(c2ccccc2)c2ccc3c(c2)c2cc(N(c4ccccc4)c4ccccc4)ccc2n3-c2ccccc2)cc1]

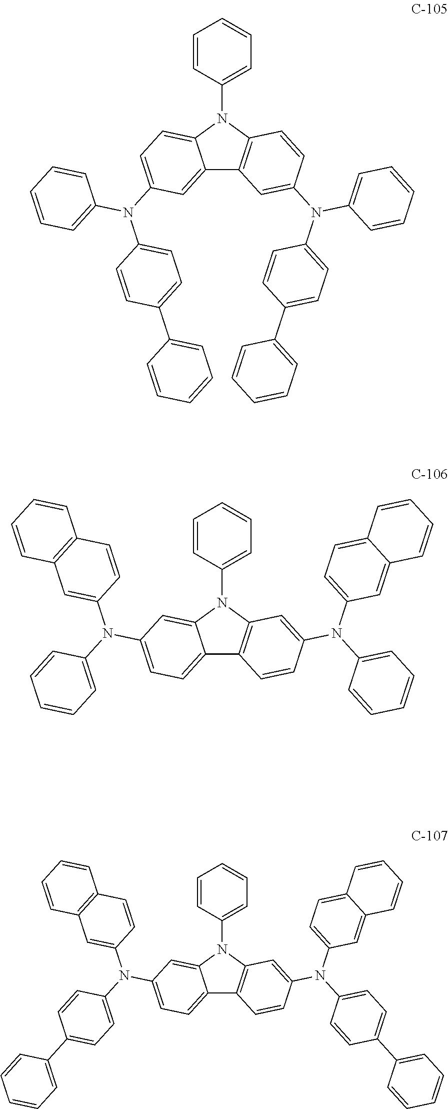 [SMILES: c1ccc(-c2ccc(N(c3ccccc3)c3ccc4c(c3)c3cc(N(c5ccccc5)c5ccc(-c6ccccc6)cc5)ccc3n4-c3ccccc3)cc2)cc1 ; c1ccc(N(c2ccc3ccccc3c2)c2ccc3c4ccc(N(c5ccccc5)c5ccc6ccccc6c5)cc4n(-c4ccccc4)c3c2)cc1 ; c1ccc(-c2ccc(N(c3ccc4ccccc4c3)c3ccc4c5ccc(N(c6ccc(-c7ccccc7)cc6)c6ccc7ccccc7c6)cc5n(-c5ccccc5)c4c3)cc2)cc1]

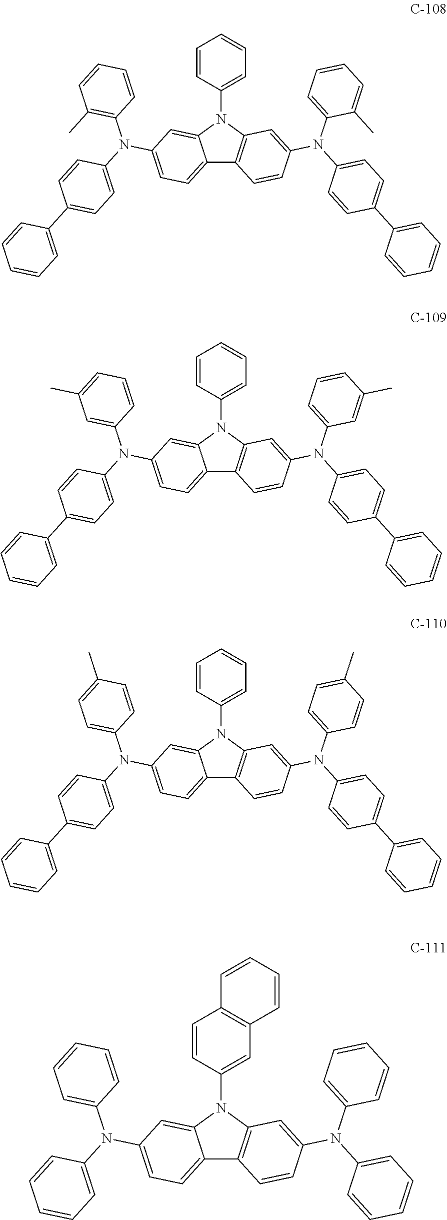 [SMILES: Cc1ccccc1N(c1ccc(-c2ccccc2)cc1)c1ccc2c3ccc(N(c4ccc(-c5ccccc5)cc4)c4ccccc4C)cc3n(-c3ccccc3)c2c1 ; Cc1cccc(N(c2ccc(-c3ccccc3)cc2)c2ccc3c4ccc(N(c5ccc(-c6ccccc6)cc5)c5cccc(C)c5)cc4n(-c4ccccc4)c3c2)c1 ; Cc1ccc(N(c2ccc(-c3ccccc3)cc2)c2ccc3c4ccc(N(c5ccc(C)cc5)c5ccc(-c6ccccc6)cc5)cc4n(-c4ccccc4)c3c2)cc1 ; c1ccc(N(c2ccccc2)c2ccc3c4ccc(N(c5ccccc5)c5ccccc5)cc4n(-c4ccc5ccccc5c4)c3c2)cc1]

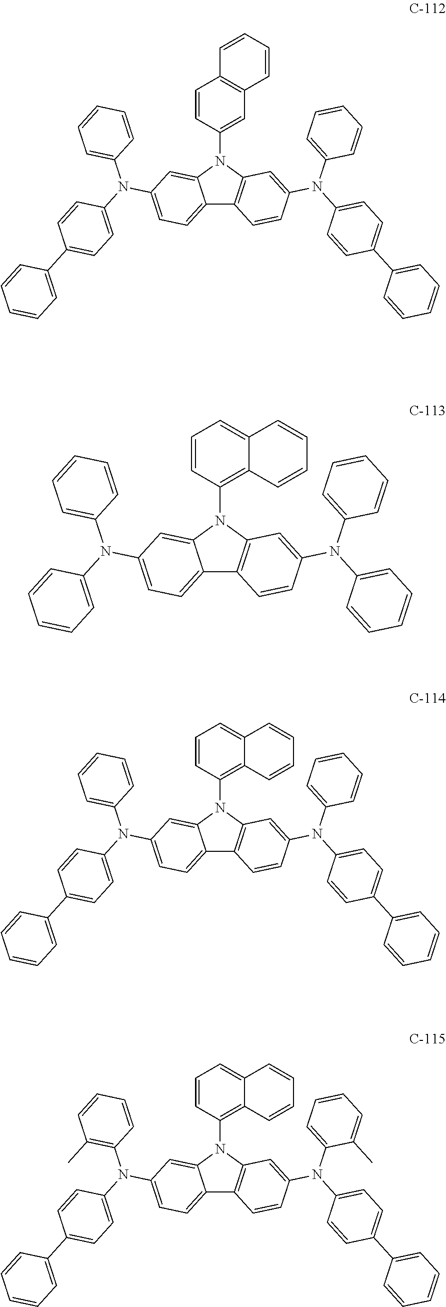 [SMILES: c1ccc(-c2ccc(N(c3ccccc3)c3ccc4c5ccc(N(c6ccccc6)c6ccc(-c7ccccc7)cc6)cc5n(-c5ccc6ccccc6c5)c4c3)cc2)cc1 ; c1ccc(N(c2ccccc2)c2ccc3c4ccc(N(c5ccccc5)c5ccccc5)cc4n(-c4cccc5ccccc45)c3c2)cc1 ; c1ccc(-c2ccc(N(c3ccccc3)c3ccc4c5ccc(N(c6ccccc6)c6ccc(-c7ccccc7)cc6)cc5n(-c5cccc6ccccc56)c4c3)cc2)cc1 ; Cc1ccccc1N(c1ccc(-c2ccccc2)cc1)c1ccc2c3ccc(N(c4ccc(-c5ccccc5)cc4)c4ccccc4C)cc3n(-c3cccc4ccccc34)c2c1]

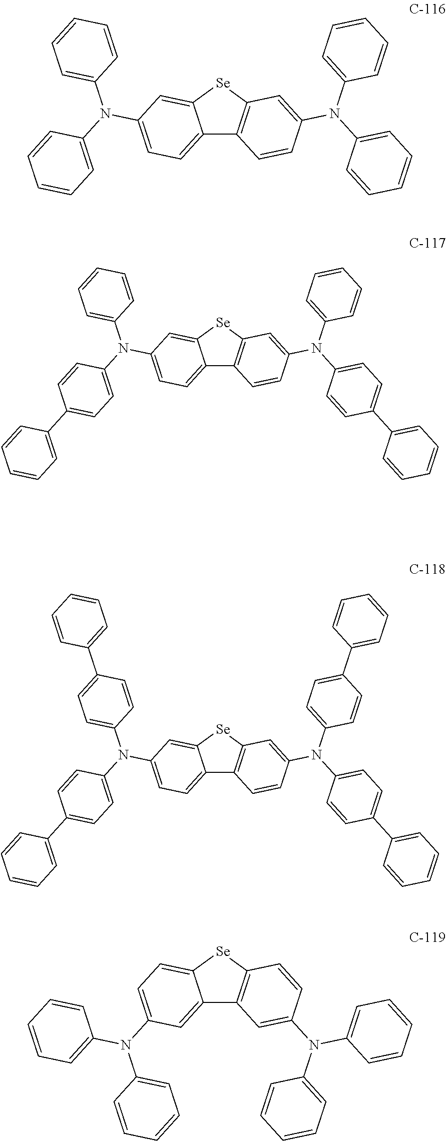 [SMILES: c1ccc(N(c2ccccc2)c2ccc3c(c2)[se]c2cc(N(c4ccccc4)c4ccccc4)ccc23)cc1 ; c1ccc(-c2ccc(N(c3ccccc3)c3ccc4c(c3)[se]c3cc(N(c5ccccc5)c5ccc(-c6ccccc6)cc5)ccc34)cc2)cc1 ; c1ccc(-c2ccc(N(c3ccc(-c4ccccc4)cc3)c3ccc4c(c3)[se]c3cc(N(c5ccc(-c6ccccc6)cc5)c5ccc(-c6ccccc6)cc5)ccc34)cc2)cc1 ; c1ccc(N(c2ccccc2)c2ccc3[se]c4ccc(N(c5ccccc5)c5ccccc5)cc4c3c2)cc1]

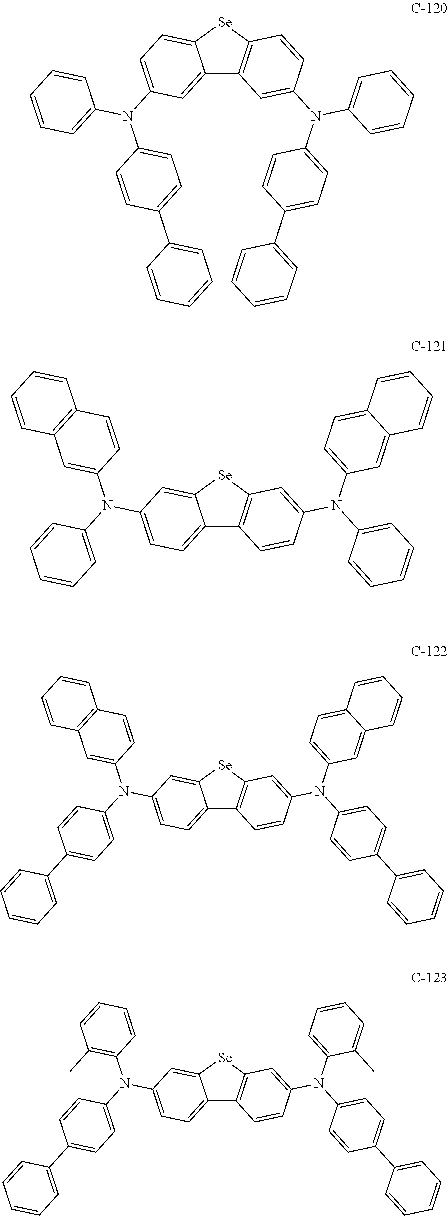 [SMILES: c1ccc(-c2ccc(N(c3ccccc3)c3ccc4[se]c5ccc(N(c6ccccc6)c6ccc(-c7ccccc7)cc6)cc5c4c3)cc2)cc1 ; c1ccc(N(c2ccc3ccccc3c2)c2ccc3c(c2)[se]c2cc(N(c4ccccc4)c4ccc5ccccc5c4)ccc23)cc1 ; c1ccc(-c2ccc(N(c3ccc4ccccc4c3)c3ccc4c(c3)[se]c3cc(N(c5ccc(-c6ccccc6)cc5)c5ccc6ccccc6c5)ccc34)cc2)cc1 ; Cc1ccccc1N(c1ccc(-c2ccccc2)cc1)c1ccc2c(c1)[se]c1cc(N(c3ccc(-c4ccccc4)cc3)c3ccccc3C)ccc12]

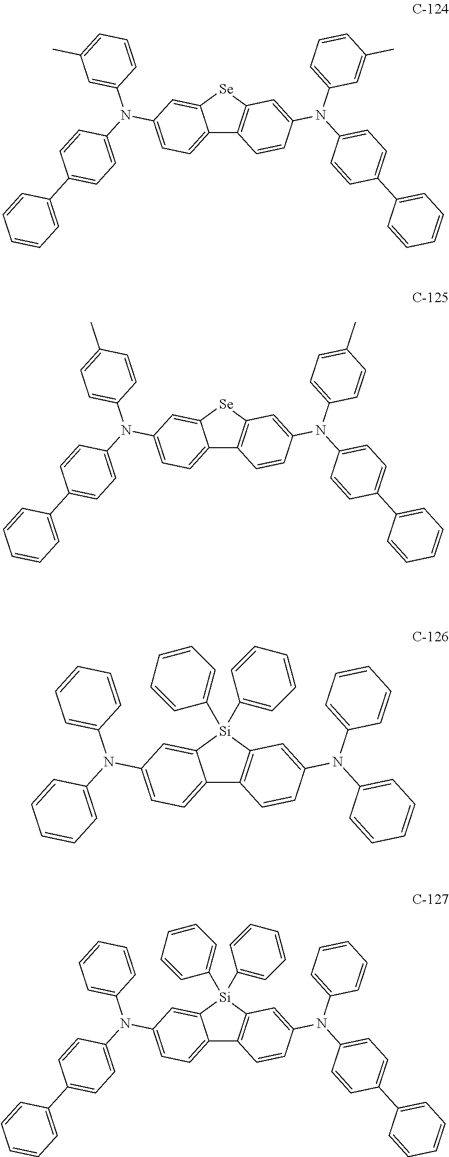 [SMILES: Cc1cccc(N(c2ccc(-c3ccccc3)cc2)c2ccc3c(c2)[se]c2cc(N(c4ccc(-c5ccccc5)cc4)c4cccc(C)c4)ccc23)c1 ; Cc1ccc(N(c2ccc(-c3ccccc3)cc2)c2ccc3c(c2)[se]c2cc(N(c4ccc(C)cc4)c4ccc(-c5ccccc5)cc4)ccc23)cc1 ; c1ccc(N(c2ccccc2)c2ccc3c(c2)[Si](c2ccccc2)(c2ccccc2)c2cc(N(c4ccccc4)c4ccccc4)ccc2-3)cc1 ; c1ccc(-c2ccc(N(c3ccccc3)c3ccc4c(c3)[Si](c3ccccc3)(c3ccccc3)c3cc(N(c5ccccc5)c5ccc(-c6ccccc6)cc5)ccc3-4)cc2)cc1]

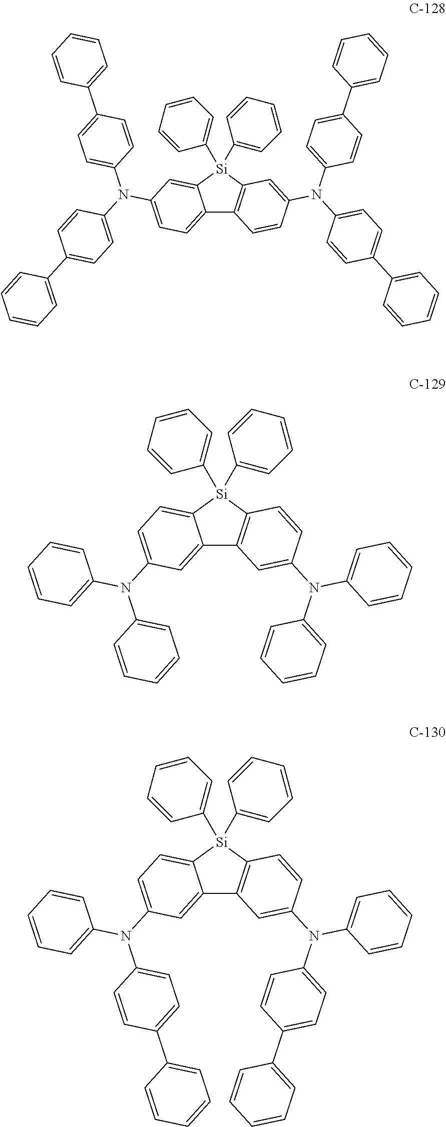 [SMILES: c1ccc(-c2ccc(N(c3ccc(-c4ccccc4)cc3)c3ccc4c(c3)[Si](c3ccccc3)(c3ccccc3)c3cc(N(c5ccc(-c6ccccc6)cc5)c5ccc(-c6ccccc6)cc5)ccc3-4)cc2)cc1 ; c1ccc(N(c2ccccc2)c2ccc3c(c2)-c2cc(N(c4ccccc4)c4ccccc4)ccc2[Si]3(c2ccccc2)c2ccccc2)cc1 ; c1ccc(-c2ccc(N(c3ccccc3)c3ccc4c(c3)-c3cc(N(c5ccccc5)c5ccc(-c6ccccc6)cc5)ccc3[Si]4(c3ccccc3)c3ccccc3)cc2)cc1]

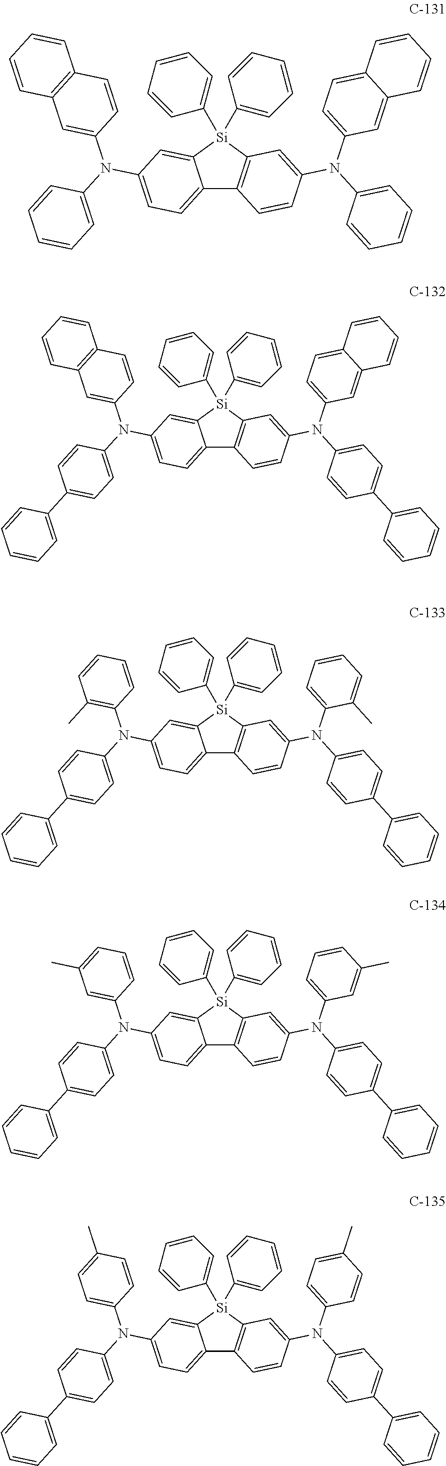

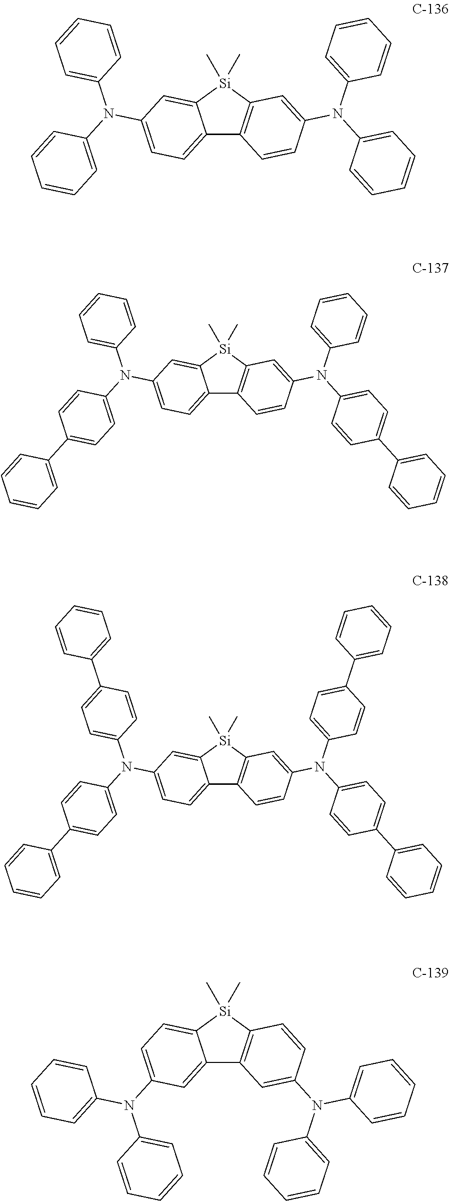 [SMILES: C[Si]1(C)c2cc(N(c3ccccc3)c3ccccc3)ccc2-c2ccc(N(c3ccccc3)c3ccccc3)cc21 ; C[Si]1(C)c2cc(N(c3ccccc3)c3ccc(-c4ccccc4)cc3)ccc2-c2ccc(N(c3ccccc3)c3ccc(-c4ccccc4)cc3)cc21 ; C[Si]1(C)c2cc(N(c3ccc(-c4ccccc4)cc3)c3ccc(-c4ccccc4)cc3)ccc2-c2ccc(N(c3ccc(-c4ccccc4)cc3)c3ccc(-c4ccccc4)cc3)cc21 ; C[Si]1(C)c2ccc(N(c3ccccc3)c3ccccc3)cc2-c2cc(N(c3ccccc3)c3ccccc3)ccc21]

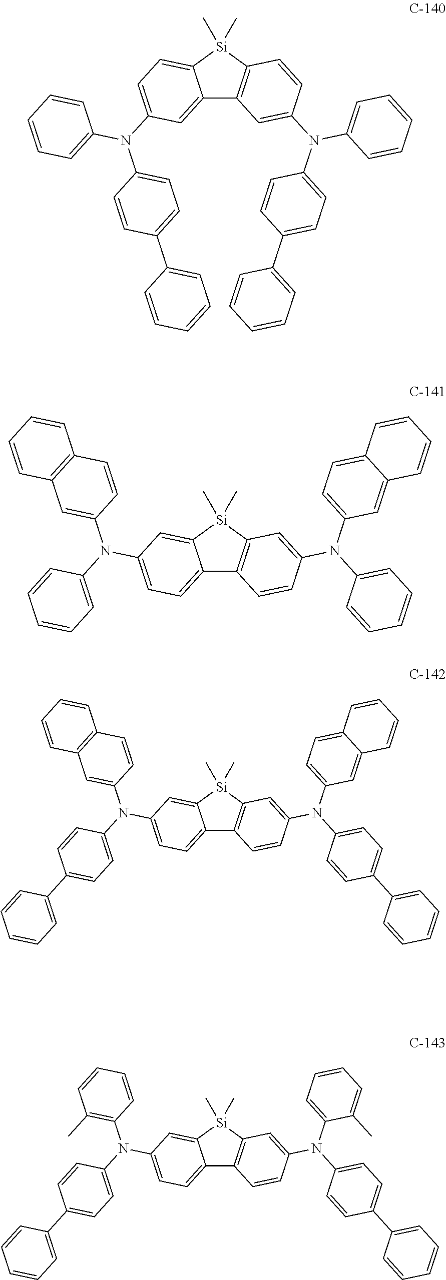 [SMILES: C[Si]1(C)c2ccc(N(c3ccccc3)c3ccc(-c4ccccc4)cc3)cc2-c2cc(N(c3ccccc3)c3ccc(-c4ccccc4)cc3)ccc21 ; C[Si]1(C)c2cc(N(c3ccccc3)c3ccc4ccccc4c3)ccc2-c2ccc(N(c3ccccc3)c3ccc4ccccc4c3)cc21 ; C[Si]1(C)c2cc(N(c3ccc(-c4ccccc4)cc3)c3ccc4ccccc4c3)ccc2-c2ccc(N(c3ccc(-c4ccccc4)cc3)c3ccc4ccccc4c3)cc21 ; Cc1ccccc1N(c1ccc(-c2ccccc2)cc1)c1ccc2c(c1)[Si](C)(C)c1cc(N(c3ccc(-c4ccccc4)cc3)c3ccccc3C)ccc1-2]

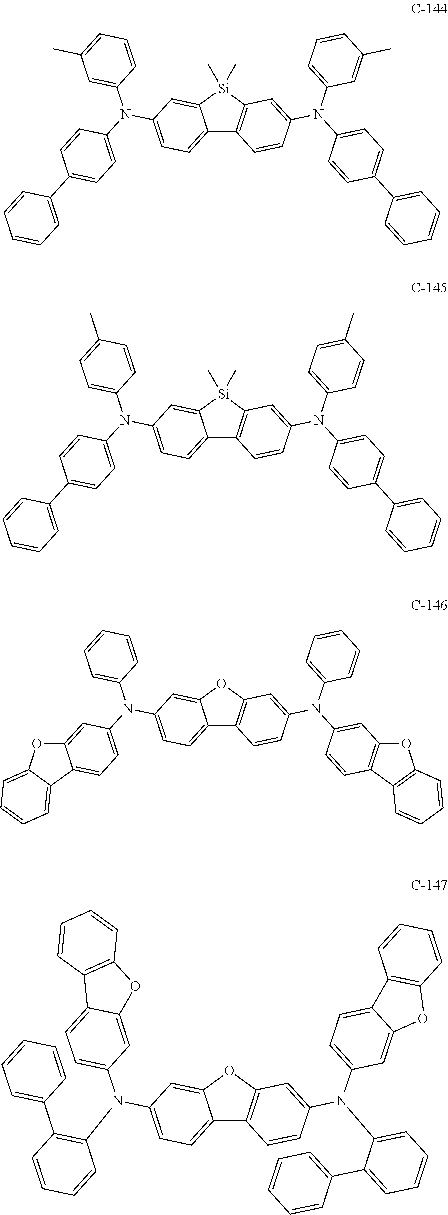 [SMILES: Cc1cccc(N(c2ccc(-c3ccccc3)cc2)c2ccc3c(c2)[Si](C)(C)c2cc(N(c4ccc(-c5ccccc5)cc4)c4cccc(C)c4)ccc2-3)c1 ; Cc1ccc(N(c2ccc(-c3ccccc3)cc2)c2ccc3c(c2)[Si](C)(C)c2cc(N(c4ccc(C)cc4)c4ccc(-c5ccccc5)cc4)ccc2-3)cc1 ; c1ccc(N(c2ccc3c(c2)oc2ccccc23)c2ccc3c(c2)oc2cc(N(c4ccccc4)c4ccc5c(c4)oc4ccccc45)ccc23)cc1 ; c1ccc(-c2ccccc2N(c2ccc3c(c2)oc2ccccc23)c2ccc3c(c2)oc2cc(N(c4ccc5c(c4)oc4ccccc45)c4ccccc4-c4ccccc4)ccc23)cc1]

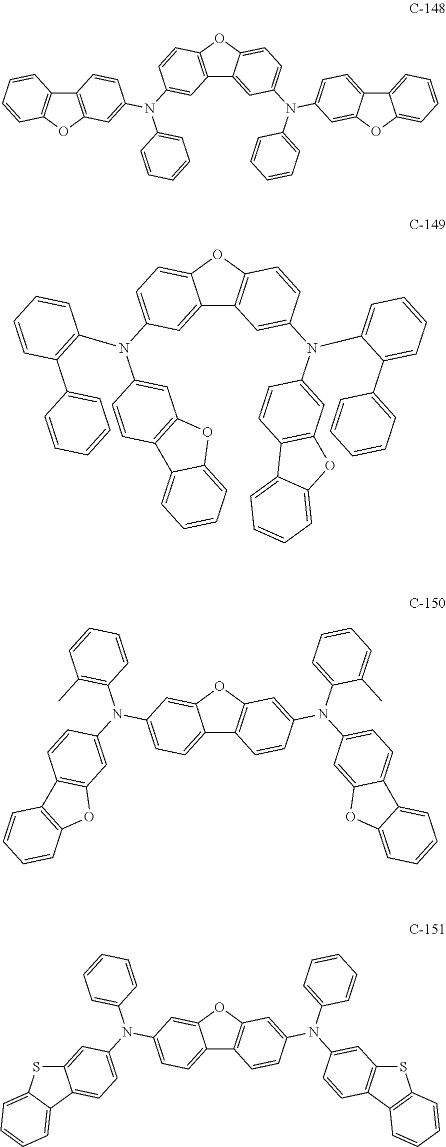 [SMILES: c1ccc(N(c2ccc3c(c2)oc2ccccc23)c2ccc3oc4ccc(N(c5ccccc5)c5ccc6c(c5)oc5ccccc56)cc4c3c2)cc1 ; c1ccc(-c2ccccc2N(c2ccc3c(c2)oc2ccccc23)c2ccc3oc4ccc(N(c5ccc6c(c5)oc5ccccc56)c5ccccc5-c5ccccc5)cc4c3c2)cc1 ; Cc1ccccc1N(c1ccc2c(c1)oc1ccccc12)c1ccc2c(c1)oc1cc(N(c3ccc4c(c3)oc3ccccc34)c3ccccc3C)ccc12 ; c1ccc(N(c2ccc3c(c2)oc2cc(N(c4ccccc4)c4ccc5c(c4)sc4ccccc45)ccc23)c2ccc3c(c2)sc2ccccc23)cc1]

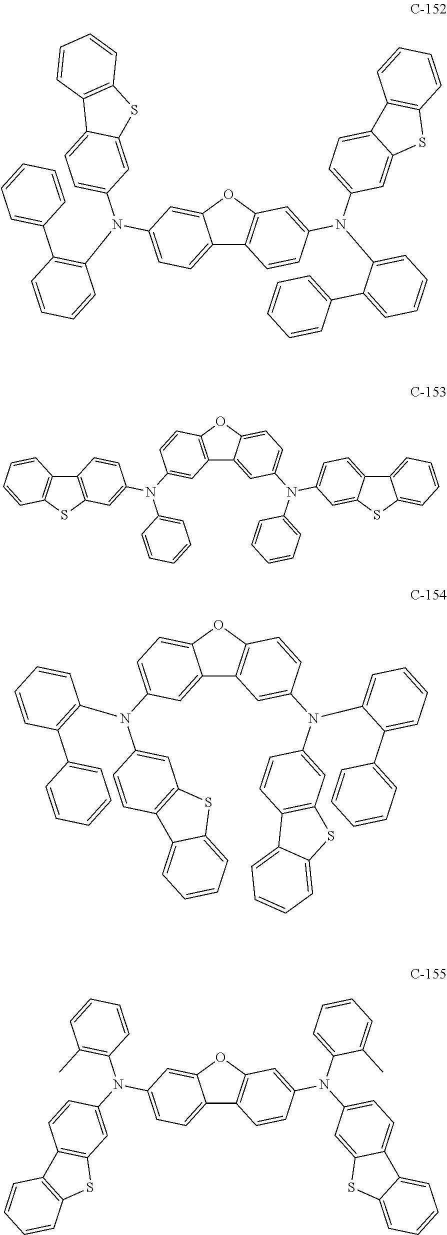 [SMILES: c1ccc(-c2ccccc2N(c2ccc3c(c2)oc2cc(N(c4ccc5c(c4)sc4ccccc45)c4ccccc4-c4ccccc4)ccc23)c2ccc3c(c2)sc2ccccc23)cc1 ; c1ccc(N(c2ccc3c(c2)sc2ccccc23)c2ccc3oc4ccc(N(c5ccccc5)c5ccc6c(c5)sc5ccccc56)cc4c3c2)cc1 ; c1ccc(-c2ccccc2N(c2ccc3c(c2)sc2ccccc23)c2ccc3oc4ccc(N(c5ccc6c(c5)sc5ccccc56)c5ccccc5-c5ccccc5)cc4c3c2)cc1 ; Cc1ccccc1N(c1ccc2c(c1)oc1cc(N(c3ccc4c(c3)sc3ccccc34)c3ccccc3C)ccc12)c1ccc2c(c1)sc1ccccc12]

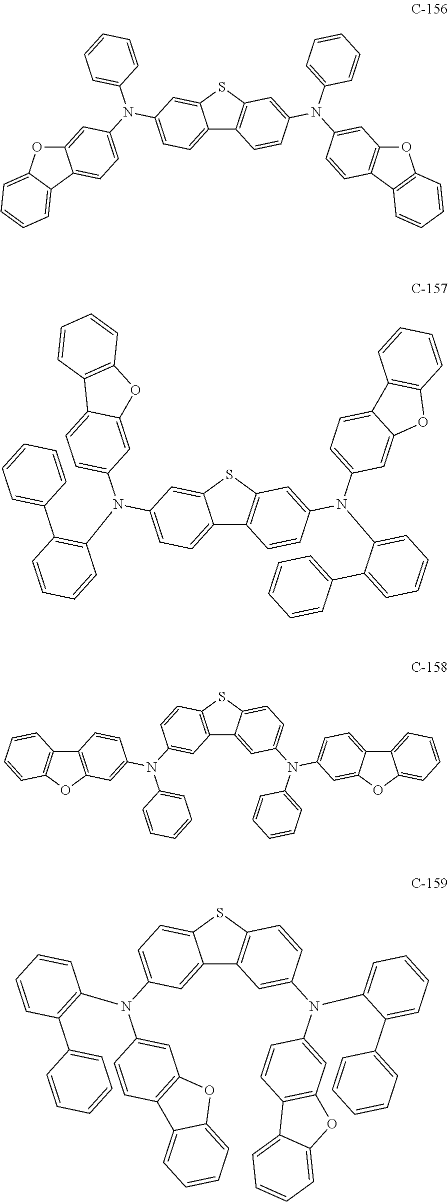 [SMILES: c1ccc(N(c2ccc3c(c2)oc2ccccc23)c2ccc3c(c2)sc2cc(N(c4ccccc4)c4ccc5c(c4)oc4ccccc45)ccc23)cc1 ; c1ccc(-c2ccccc2N(c2ccc3c(c2)oc2ccccc23)c2ccc3c(c2)sc2cc(N(c4ccc5c(c4)oc4ccccc45)c4ccccc4-c4ccccc4)ccc23)cc1 ; c1ccc(N(c2ccc3c(c2)oc2ccccc23)c2ccc3sc4ccc(N(c5ccccc5)c5ccc6c(c5)oc5ccccc56)cc4c3c2)cc1 ; c1ccc(-c2ccccc2N(c2ccc3c(c2)oc2ccccc23)c2ccc3sc4ccc(N(c5ccc6c(c5)oc5ccccc56)c5ccccc5-c5ccccc5)cc4c3c2)cc1]

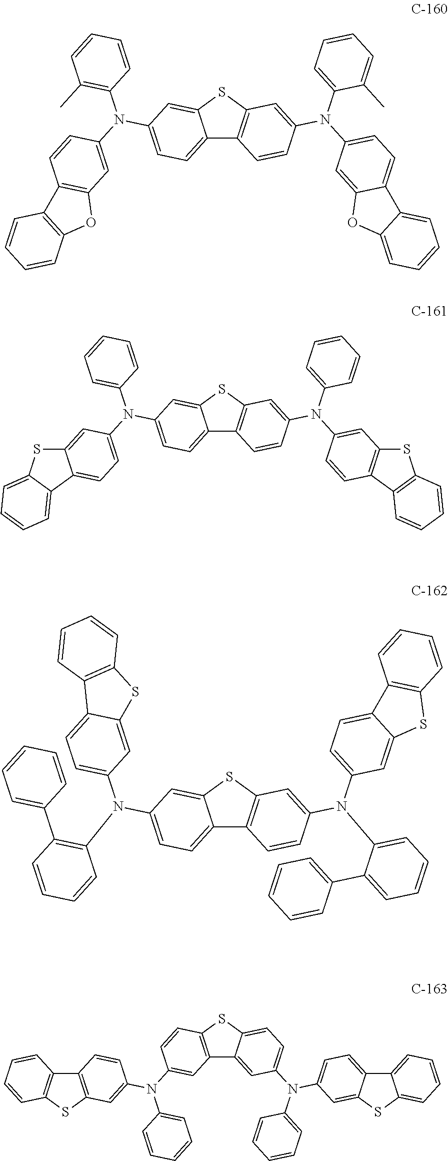 [SMILES: Cc1ccccc1N(c1ccc2c(c1)oc1ccccc12)c1ccc2c(c1)sc1cc(N(c3ccc4c(c3)oc3ccccc34)c3ccccc3C)ccc12 ; c1ccc(N(c2ccc3c(c2)sc2ccccc23)c2ccc3c(c2)sc2cc(N(c4ccccc4)c4ccc5c(c4)sc4ccccc45)ccc23)cc1 ; c1ccc(-c2ccccc2N(c2ccc3c(c2)sc2ccccc23)c2ccc3c(c2)sc2cc(N(c4ccc5c(c4)sc4ccccc45)c4ccccc4-c4ccccc4)ccc23)cc1 ; c1ccc(N(c2ccc3c(c2)sc2ccccc23)c2ccc3sc4ccc(N(c5ccccc5)c5ccc6c(c5)sc5ccccc56)cc4c3c2)cc1]

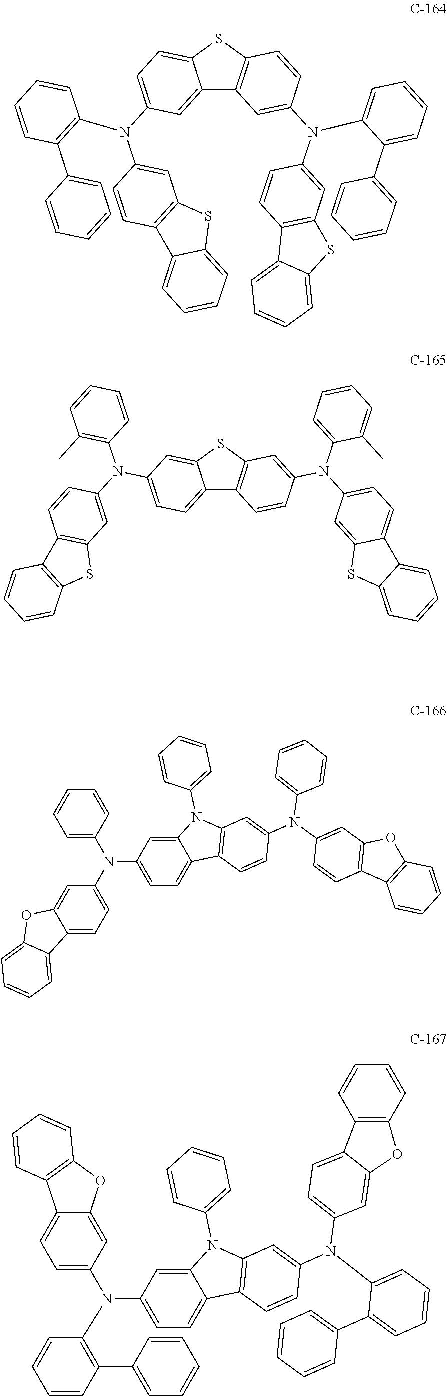 [SMILES: c1ccc(-c2ccccc2N(c2ccc3c(c2)sc2ccccc23)c2ccc3sc4ccc(N(c5ccc6c(c5)sc5ccccc56)c5ccccc5-c5ccccc5)cc4c3c2)cc1 ; Cc1ccccc1N(c1ccc2c(c1)sc1ccccc12)c1ccc2c(c1)sc1cc(N(c3ccc4c(c3)sc3ccccc34)c3ccccc3C)ccc12 ; c1ccc(N(c2ccc3c(c2)oc2ccccc23)c2ccc3c4ccc(N(c5ccccc5)c5ccc6c(c5)oc5ccccc56)cc4n(-c4ccccc4)c3c2)cc1 ; c1ccc(-c2ccccc2N(c2ccc3c(c2)oc2ccccc23)c2ccc3c4ccc(N(c5ccc6c(c5)oc5ccccc56)c5ccccc5-c5ccccc5)cc4n(-c4ccccc4)c3c2)cc1]

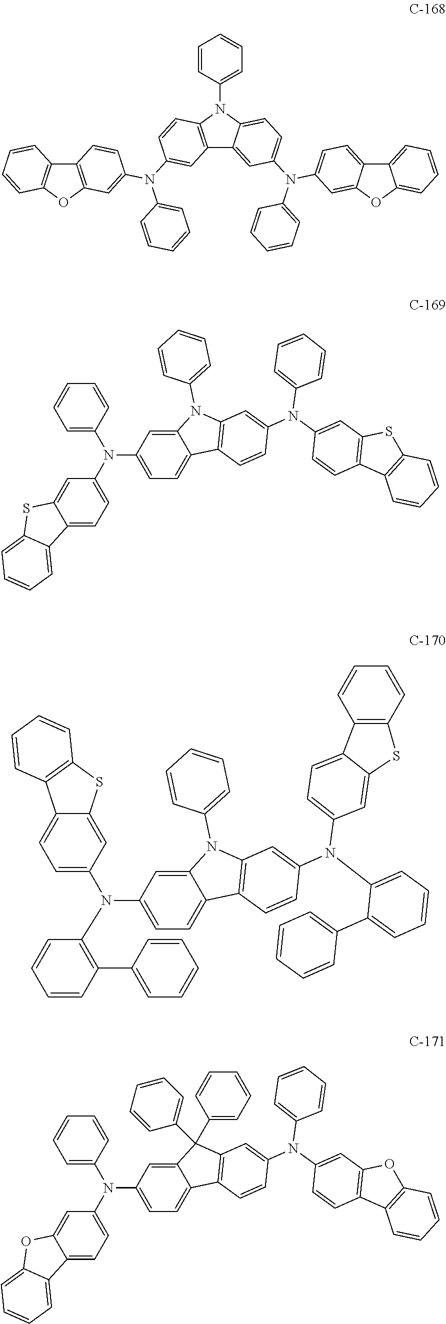 [SMILES: c1ccc(N(c2ccc3c(c2)oc2ccccc23)c2ccc3c(c2)c2cc(N(c4ccccc4)c4ccc5c(c4)oc4ccccc45)ccc2n3-c2ccccc2)cc1 ; c1ccc(N(c2ccc3c(c2)sc2ccccc23)c2ccc3c4ccc(N(c5ccccc5)c5ccc6c(c5)sc5ccccc56)cc4n(-c4ccccc4)c3c2)cc1 ; c1ccc(-c2ccccc2N(c2ccc3c(c2)sc2ccccc23)c2ccc3c4ccc(N(c5ccc6c(c5)sc5ccccc56)c5ccccc5-c5ccccc5)cc4n(-c4ccccc4)c3c2)cc1 ; c1ccc(N(c2ccc3c(c2)C(c2ccccc2)(c2ccccc2)c2cc(N(c4ccccc4)c4ccc5c(c4)oc4ccccc45)ccc2-3)c2ccc3c(c2)oc2ccccc23)cc1]

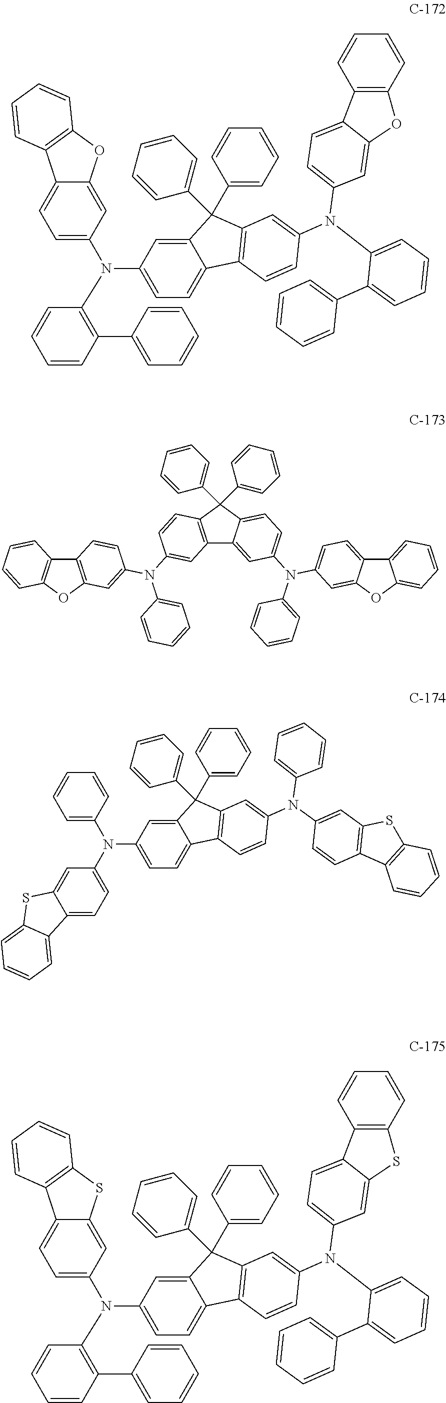 [SMILES: c1ccc(-c2ccccc2N(c2ccc3c(c2)C(c2ccccc2)(c2ccccc2)c2cc(N(c4ccc5c(c4)oc4ccccc45)c4ccccc4-c4ccccc4)ccc2-3)c2ccc3c(c2)oc2ccccc23)cc1 ; c1ccc(N(c2ccc3c(c2)-c2cc(N(c4ccccc4)c4ccc5c(c4)oc4ccccc45)ccc2C3(c2ccccc2)c2ccccc2)c2ccc3c(c2)oc2ccccc23)cc1 ; c1ccc(N(c2ccc3c(c2)C(c2ccccc2)(c2ccccc2)c2cc(N(c4ccccc4)c4ccc5c(c4)sc4ccccc45)ccc2-3)c2ccc3c(c2)sc2ccccc23)cc1 ; c1ccc(-c2ccccc2N(c2ccc3c(c2)C(c2ccccc2)(c2ccccc2)c2cc(N(c4ccc5c(c4)sc4ccccc45)c4ccccc4-c4ccccc4)ccc2-3)c2ccc3c(c2)sc2ccccc23)cc1]

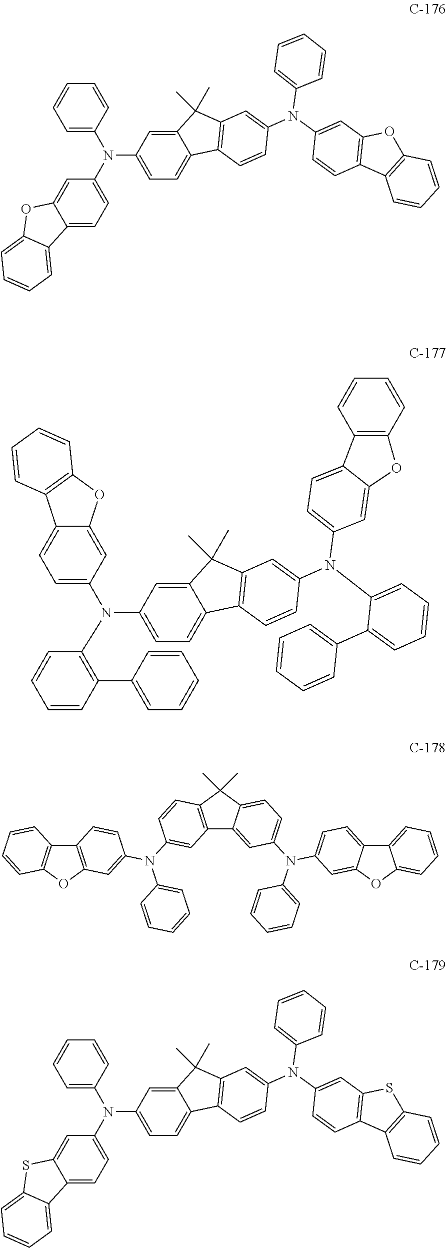 [SMILES: CC1(C)c2cc(N(c3ccccc3)c3ccc4c(c3)oc3ccccc34)ccc2-c2ccc(N(c3ccccc3)c3ccc4c(c3)oc3ccccc34)cc21 ; CC1(C)c2cc(N(c3ccc4c(c3)oc3ccccc34)c3ccccc3-c3ccccc3)ccc2-c2ccc(N(c3ccc4c(c3)oc3ccccc34)c3ccccc3-c3ccccc3)cc21 ; CC1(C)c2ccc(N(c3ccccc3)c3ccc4c(c3)oc3ccccc34)cc2-c2cc(N(c3ccccc3)c3ccc4c(c3)oc3ccccc34)ccc21 ; CC1(C)c2cc(N(c3ccccc3)c3ccc4c(c3)sc3ccccc34)ccc2-c2ccc(N(c3ccccc3)c3ccc4c(c3)sc3ccccc34)cc21]

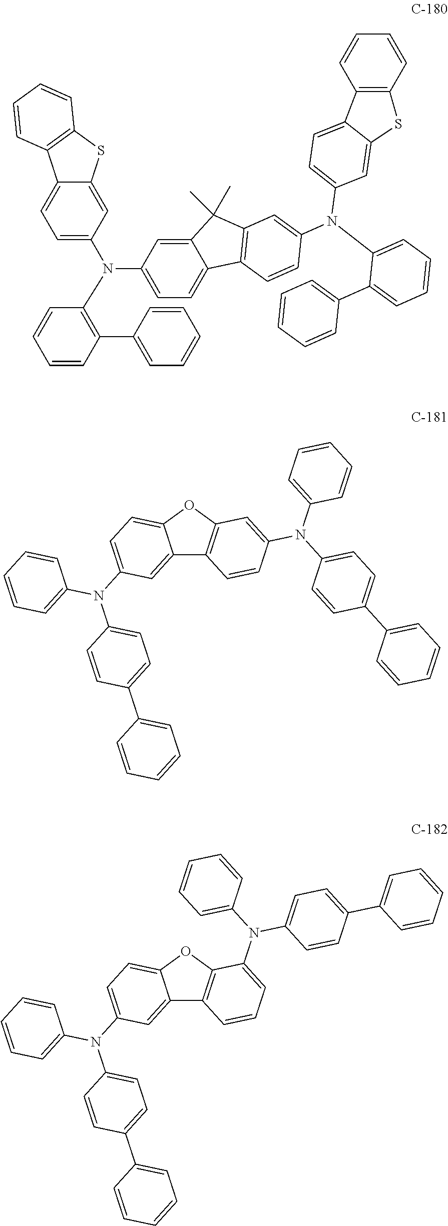 [SMILES: CC1(C)c2cc(N(c3ccc4c(c3)sc3ccccc34)c3ccccc3-c3ccccc3)ccc2-c2ccc(N(c3ccc4c(c3)sc3ccccc34)c3ccccc3-c3ccccc3)cc21 ; c1ccc(-c2ccc(N(c3ccccc3)c3ccc4c(c3)oc3ccc(N(c5ccccc5)c5ccc(-c6ccccc6)cc5)cc34)cc2)cc1 ; c1ccc(-c2ccc(N(c3ccccc3)c3ccc4oc5c(N(c6ccccc6)c6ccc(-c7ccccc7)cc6)cccc5c4c3)cc2)cc1]

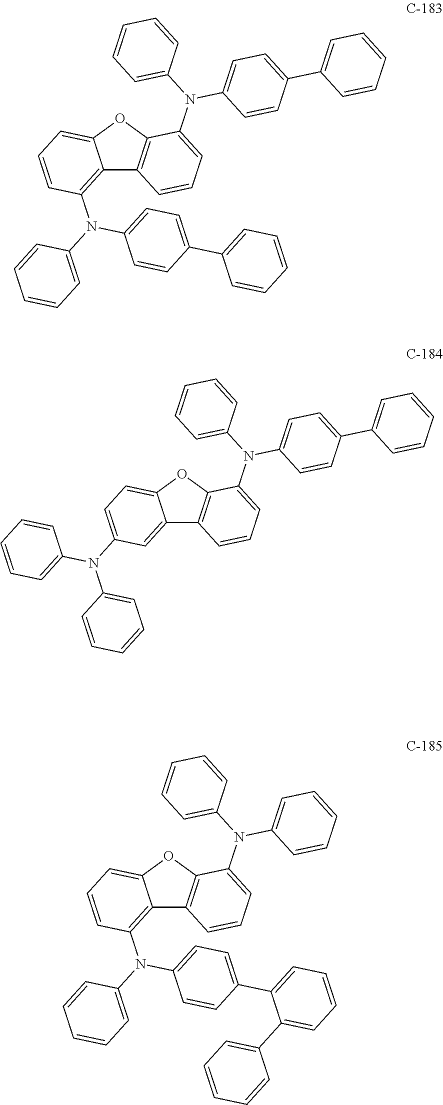 [SMILES: c1ccc(-c2ccc(N(c3ccccc3)c3cccc4c3oc3cccc(N(c5ccccc5)c5ccc(-c6ccccc6)cc5)c34)cc2)cc1 ; c1ccc(-c2ccc(N(c3ccccc3)c3cccc4c3oc3ccc(N(c5ccccc5)c5ccccc5)cc34)cc2)cc1 ; c1ccc(-c2ccccc2-c2ccc(N(c3ccccc3)c3cccc4oc5c(N(c6ccccc6)c6ccccc6)cccc5c34)cc2)cc1]

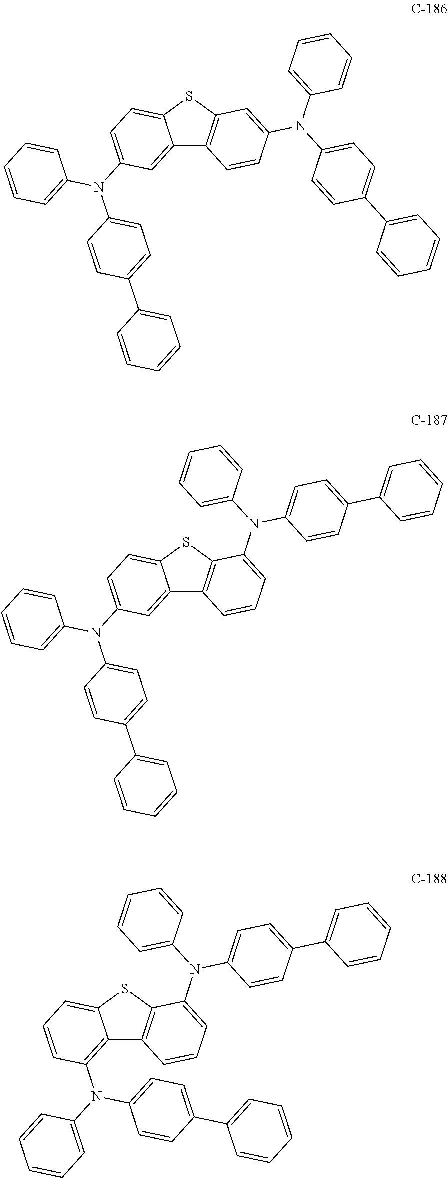 [SMILES: c1ccc(-c2ccc(N(c3ccccc3)c3ccc4c(c3)sc3ccc(N(c5ccccc5)c5ccc(-c6ccccc6)cc5)cc34)cc2)cc1 ; c1ccc(-c2ccc(N(c3ccccc3)c3ccc4sc5c(N(c6ccccc6)c6ccc(-c7ccccc7)cc6)cccc5c4c3)cc2)cc1 ; c1ccc(-c2ccc(N(c3ccccc3)c3cccc4c3sc3cccc(N(c5ccccc5)c5ccc(-c6ccccc6)cc5)c34)cc2)cc1]

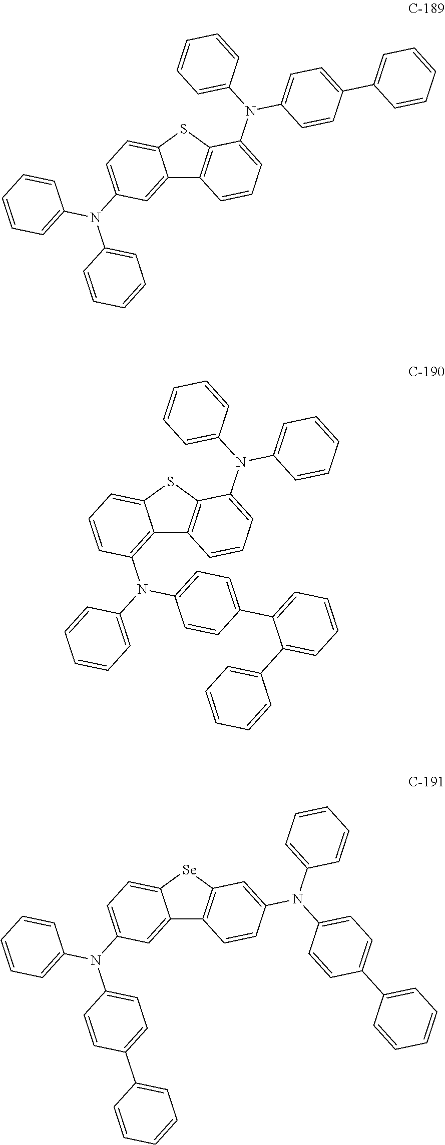 [SMILES: c1ccc(-c2ccc(N(c3ccccc3)c3cccc4c3sc3ccc(N(c5ccccc5)c5ccccc5)cc34)cc2)cc1 ; c1ccc(-c2ccccc2-c2ccc(N(c3ccccc3)c3cccc4sc5c(N(c6ccccc6)c6ccccc6)cccc5c34)cc2)cc1 ; c1ccc(-c2ccc(N(c3ccccc3)c3ccc4c(c3)[se]c3ccc(N(c5ccccc5)c5ccc(-c6ccccc6)cc5)cc34)cc2)cc1]

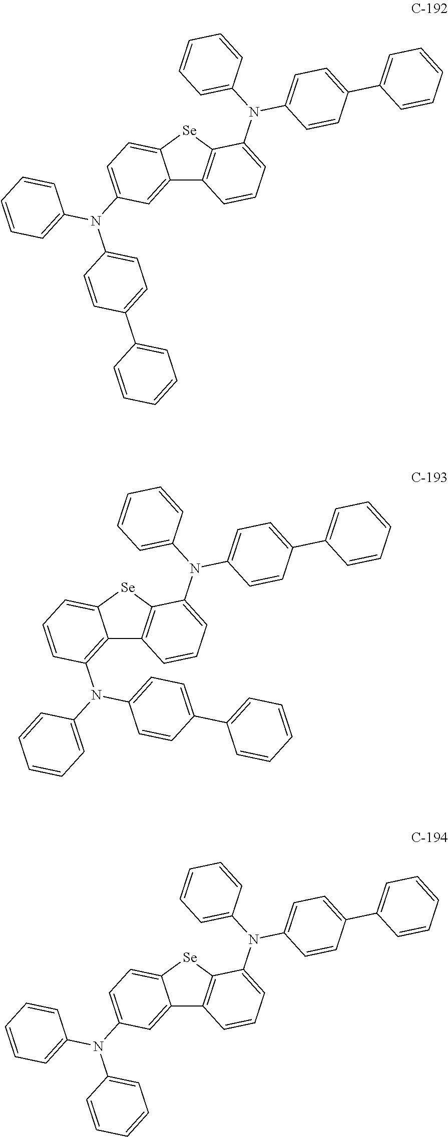 [SMILES: c1ccc(-c2ccc(N(c3ccccc3)c3ccc4[se]c5c(N(c6ccccc6)c6ccc(-c7ccccc7)cc6)cccc5c4c3)cc2)cc1 ; c1ccc(-c2ccc(N(c3ccccc3)c3cccc4c3[se]c3cccc(N(c5ccccc5)c5ccc(-c6ccccc6)cc5)c34)cc2)cc1 ; c1ccc(-c2ccc(N(c3ccccc3)c3cccc4c3[se]c3ccc(N(c5ccccc5)c5ccccc5)cc34)cc2)cc1]

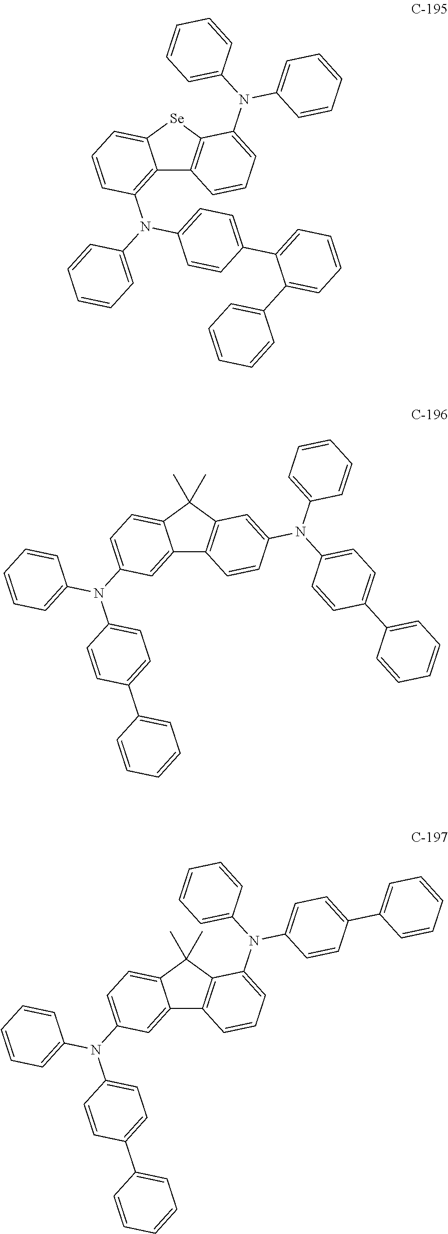 [SMILES: c1ccc(-c2ccccc2-c2ccc(N(c3ccccc3)c3cccc4[se]c5c(N(c6ccccc6)c6ccccc6)cccc5c34)cc2)cc1 ; CC1(C)c2ccc(N(c3ccccc3)c3ccc(-c4ccccc4)cc3)cc2-c2ccc(N(c3ccccc3)c3ccc(-c4ccccc4)cc3)cc21 ; CC1(C)c2ccc(N(c3ccccc3)c3ccc(-c4ccccc4)cc3)cc2-c2cccc(N(c3ccccc3)c3ccc(-c4ccccc4)cc3)c21]

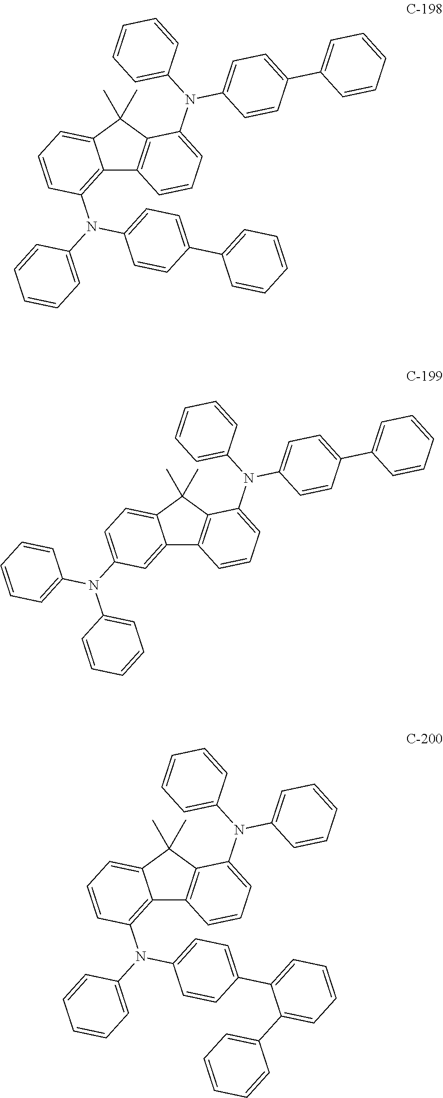 [SMILES: CC1(C)c2cccc(N(c3ccccc3)c3ccc(-c4ccccc4)cc3)c2-c2cccc(N(c3ccccc3)c3ccc(-c4ccccc4)cc3)c21 ; CC1(C)c2ccc(N(c3ccccc3)c3ccccc3)cc2-c2cccc(N(c3ccccc3)c3ccc(-c4ccccc4)cc3)c21 ; CC1(C)c2cccc(N(c3ccccc3)c3ccc(-c4ccccc4-c4ccccc4)cc3)c2-c2cccc(N(c3ccccc3)c3ccccc3)c21]

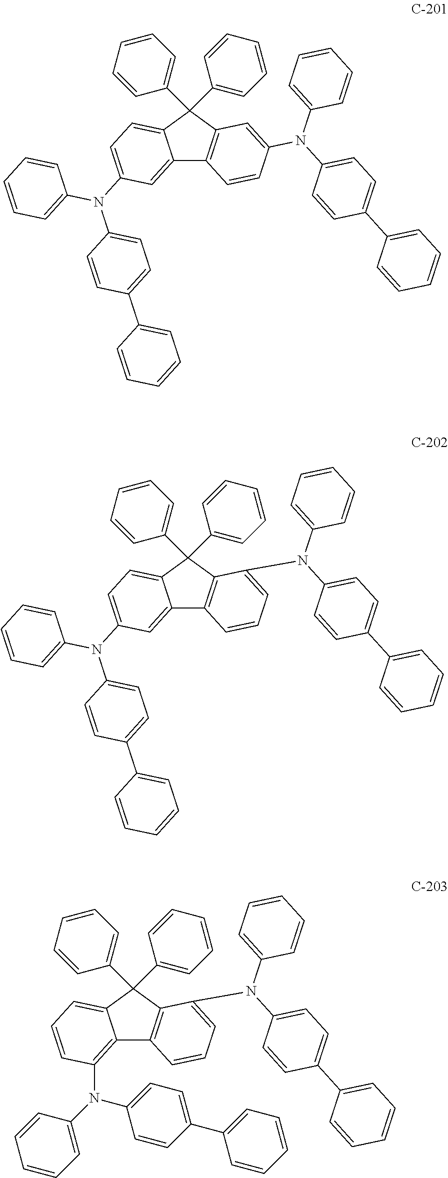 [SMILES: c1ccc(-c2ccc(N(c3ccccc3)c3ccc4c(c3)-c3ccc(N(c5ccccc5)c5ccc(-c6ccccc6)cc5)cc3C4(c3ccccc3)c3ccccc3)cc2)cc1 ; c1ccc(-c2ccc(N(c3ccccc3)c3ccc4c(c3)-c3cccc(N(c5ccccc5)c5ccc(-c6ccccc6)cc5)c3C4(c3ccccc3)c3ccccc3)cc2)cc1 ; c1ccc(-c2ccc(N(c3ccccc3)c3cccc4c3-c3cccc(N(c5ccccc5)c5ccc(-c6ccccc6)cc5)c3C4(c3ccccc3)c3ccccc3)cc2)cc1]

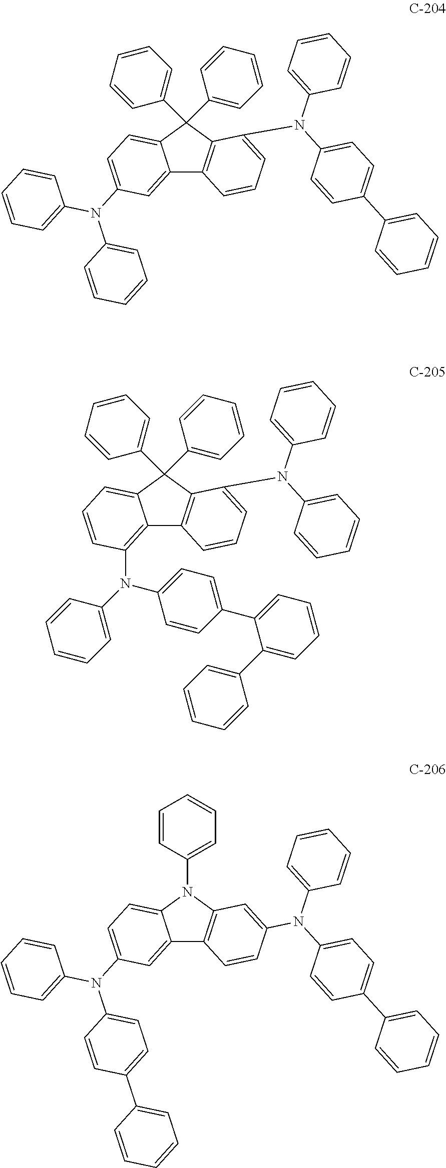 [SMILES: c1ccc(-c2ccc(N(c3ccccc3)c3cccc4c3C(c3ccccc3)(c3ccccc3)c3ccc(N(c5ccccc5)c5ccccc5)cc3-4)cc2)cc1 ; c1ccc(-c2ccccc2-c2ccc(N(c3ccccc3)c3cccc4c3-c3cccc(N(c5ccccc5)c5ccccc5)c3C4(c3ccccc3)c3ccccc3)cc2)cc1 ; c1ccc(-c2ccc(N(c3ccccc3)c3ccc4c(c3)c3ccc(N(c5ccccc5)c5ccc(-c6ccccc6)cc5)cc3n4-c3ccccc3)cc2)cc1]

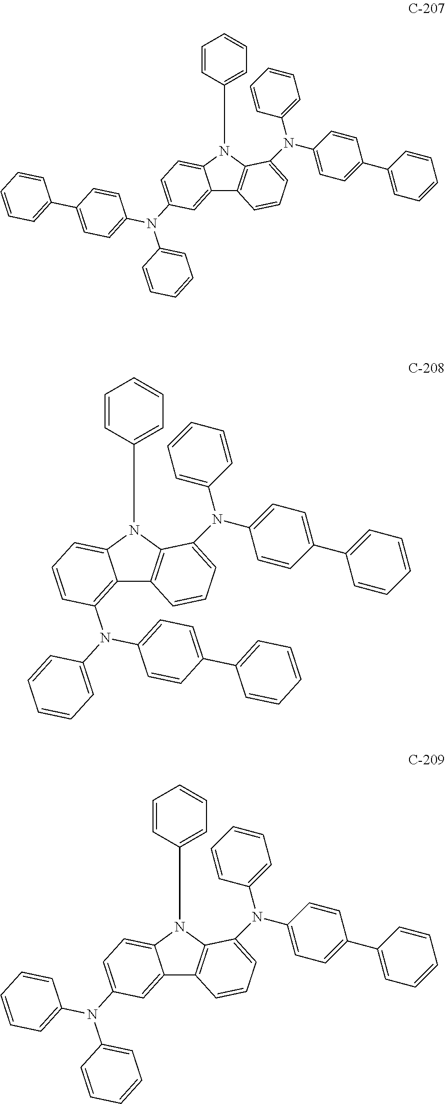 [SMILES: c1ccc(-c2ccc(N(c3ccccc3)c3ccc4c(c3)c3cccc(N(c5ccccc5)c5ccc(-c6ccccc6)cc5)c3n4-c3ccccc3)cc2)cc1 ; c1ccc(-c2ccc(N(c3ccccc3)c3cccc4c3c3cccc(N(c5ccccc5)c5ccc(-c6ccccc6)cc5)c3n4-c3ccccc3)cc2)cc1 ; c1ccc(-c2ccc(N(c3ccccc3)c3cccc4c5cc(N(c6ccccc6)c6ccccc6)ccc5n(-c5ccccc5)c34)cc2)cc1]

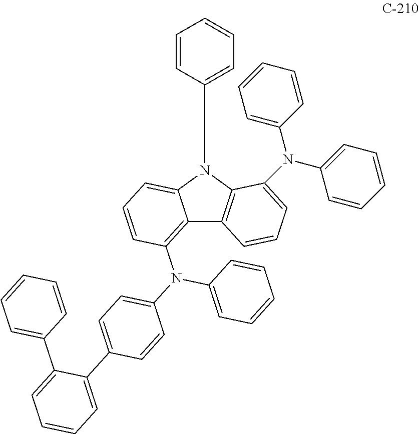 [SMILES: c1ccc(-c2ccccc2-c2ccc(N(c3ccccc3)c3cccc4c3c3cccc(N(c5ccccc5)c5ccccc5)c3n4-c3ccccc3)cc2)cc1]

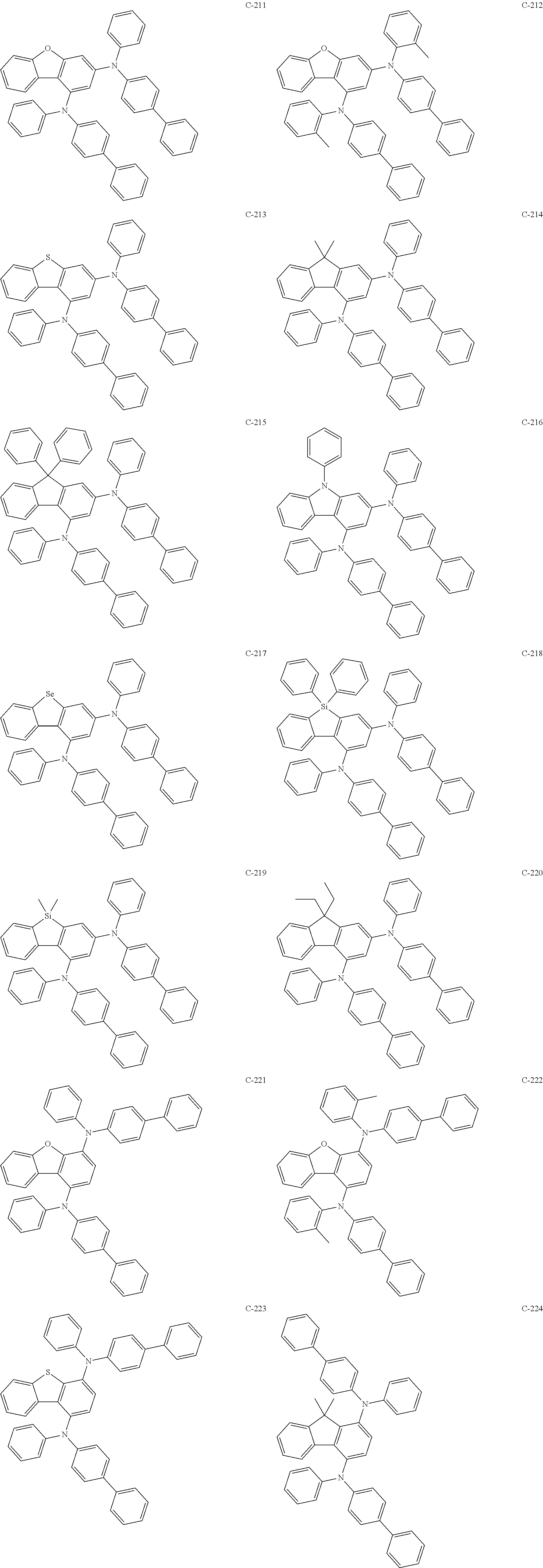

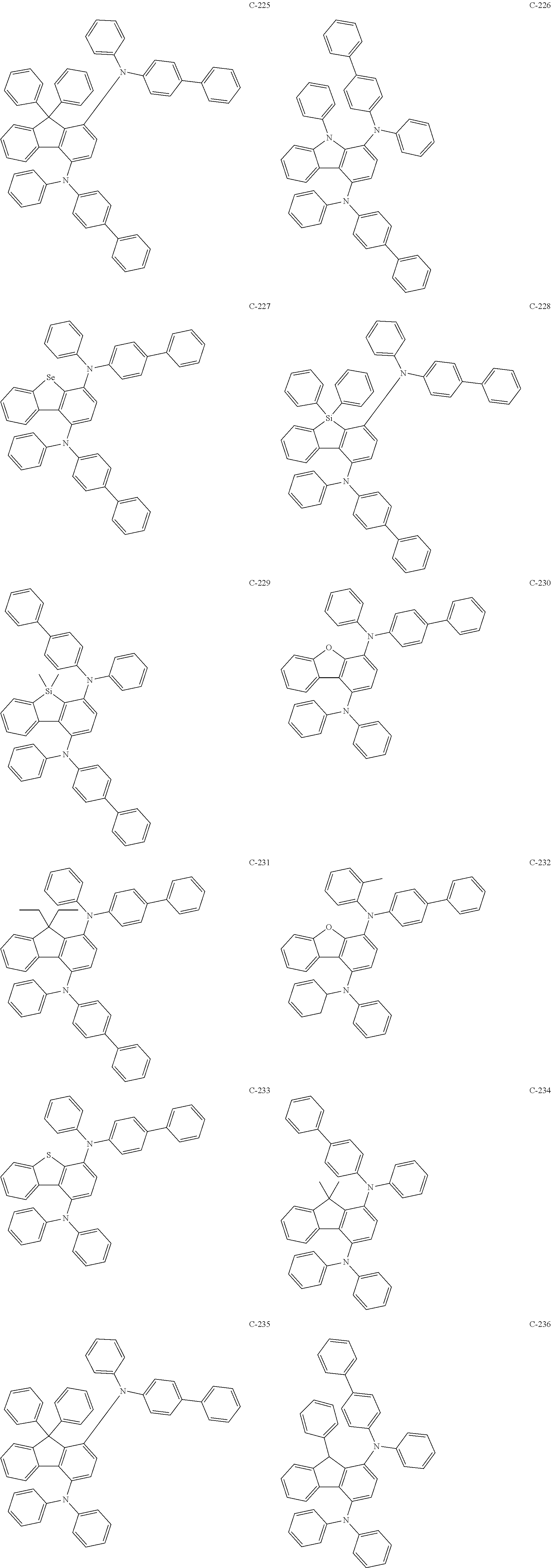

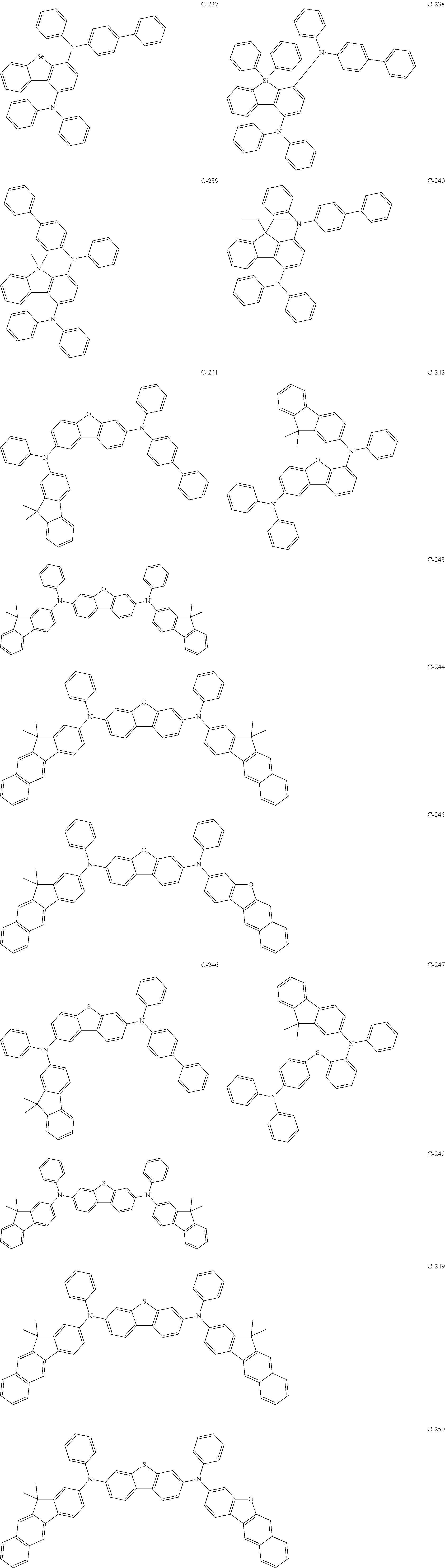

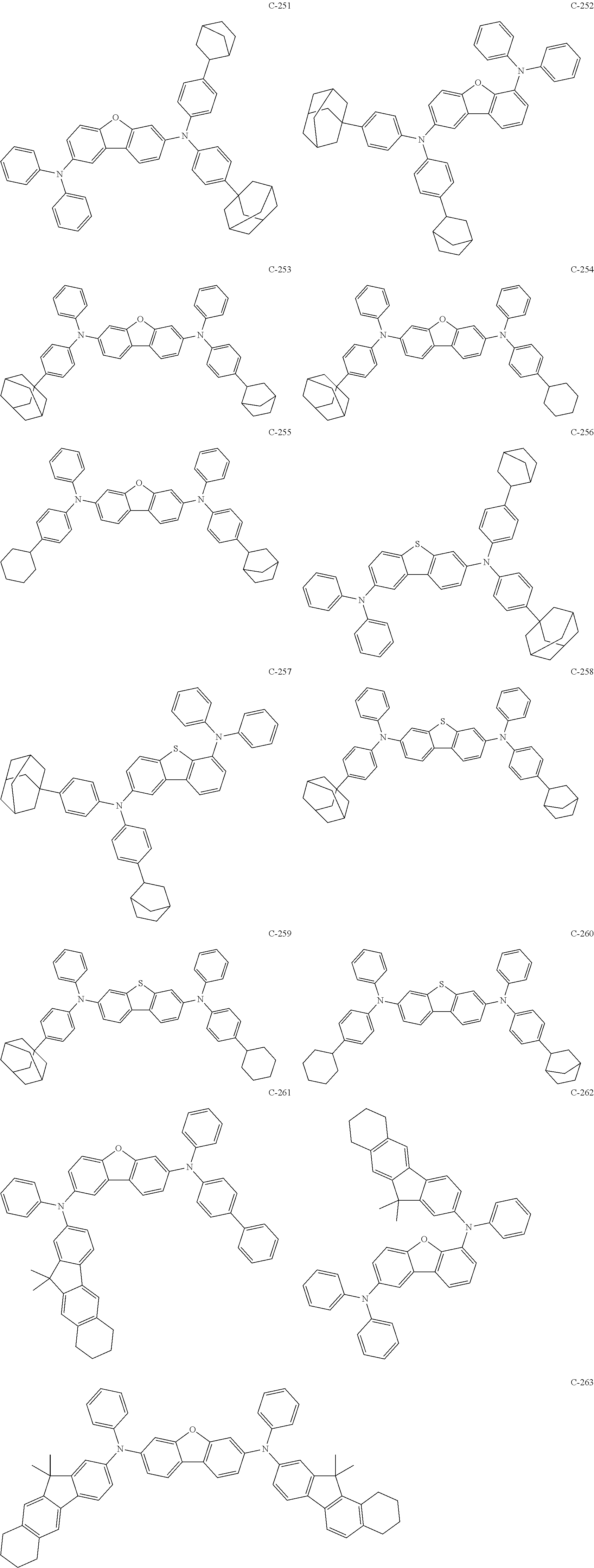

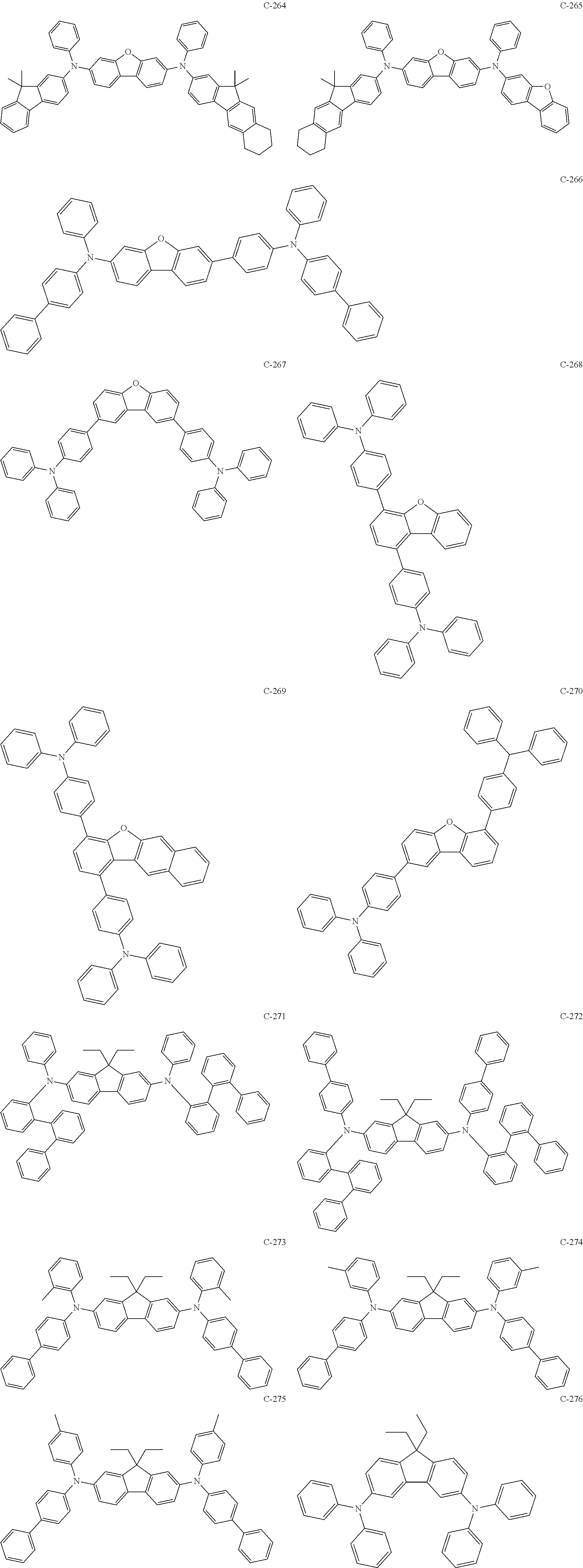

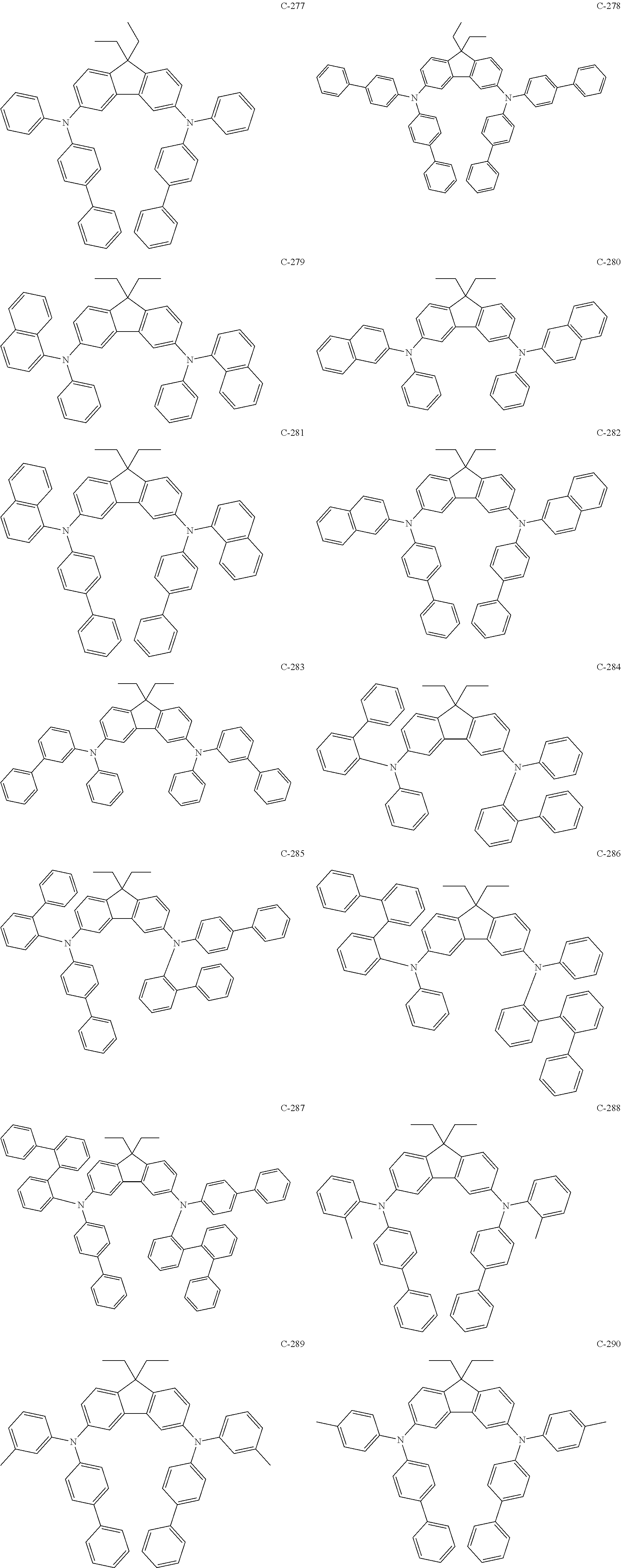

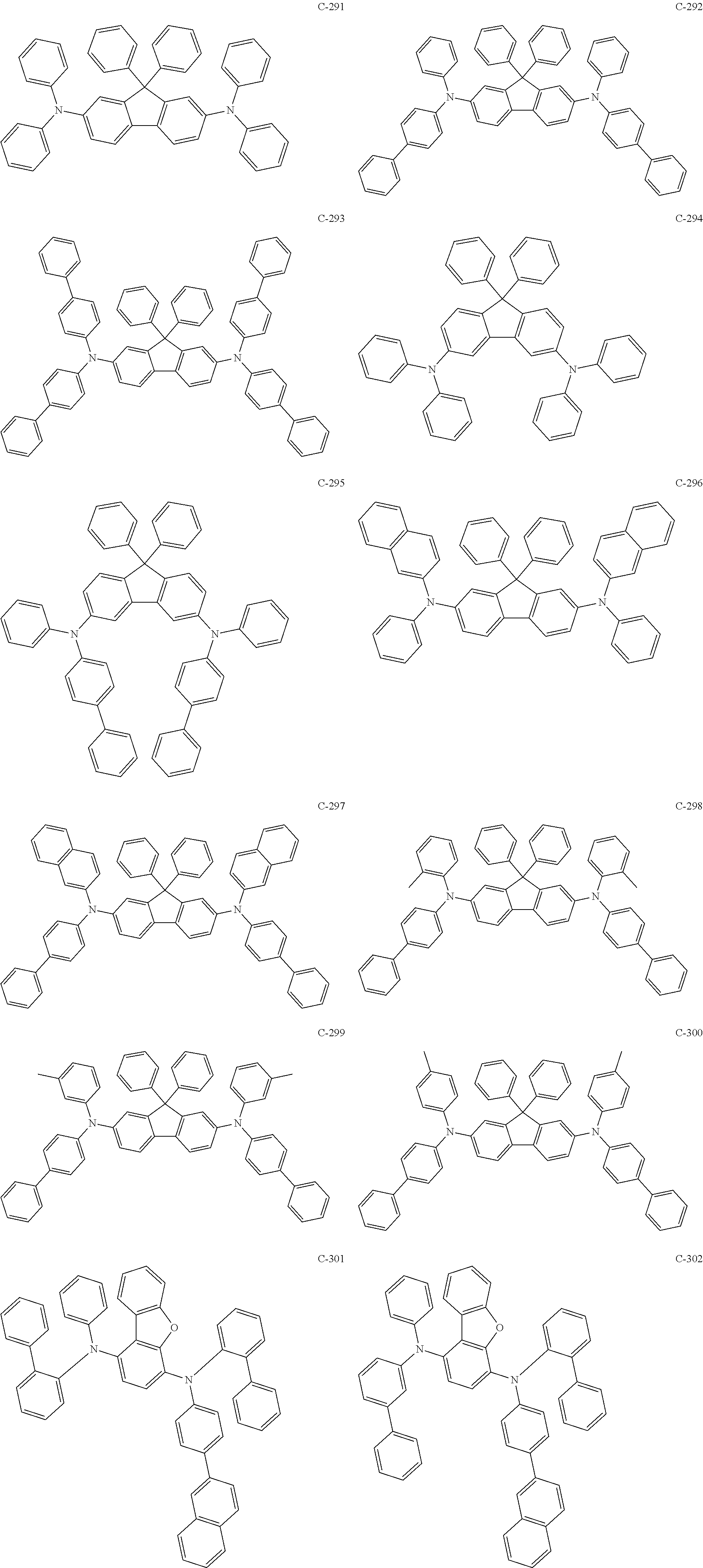

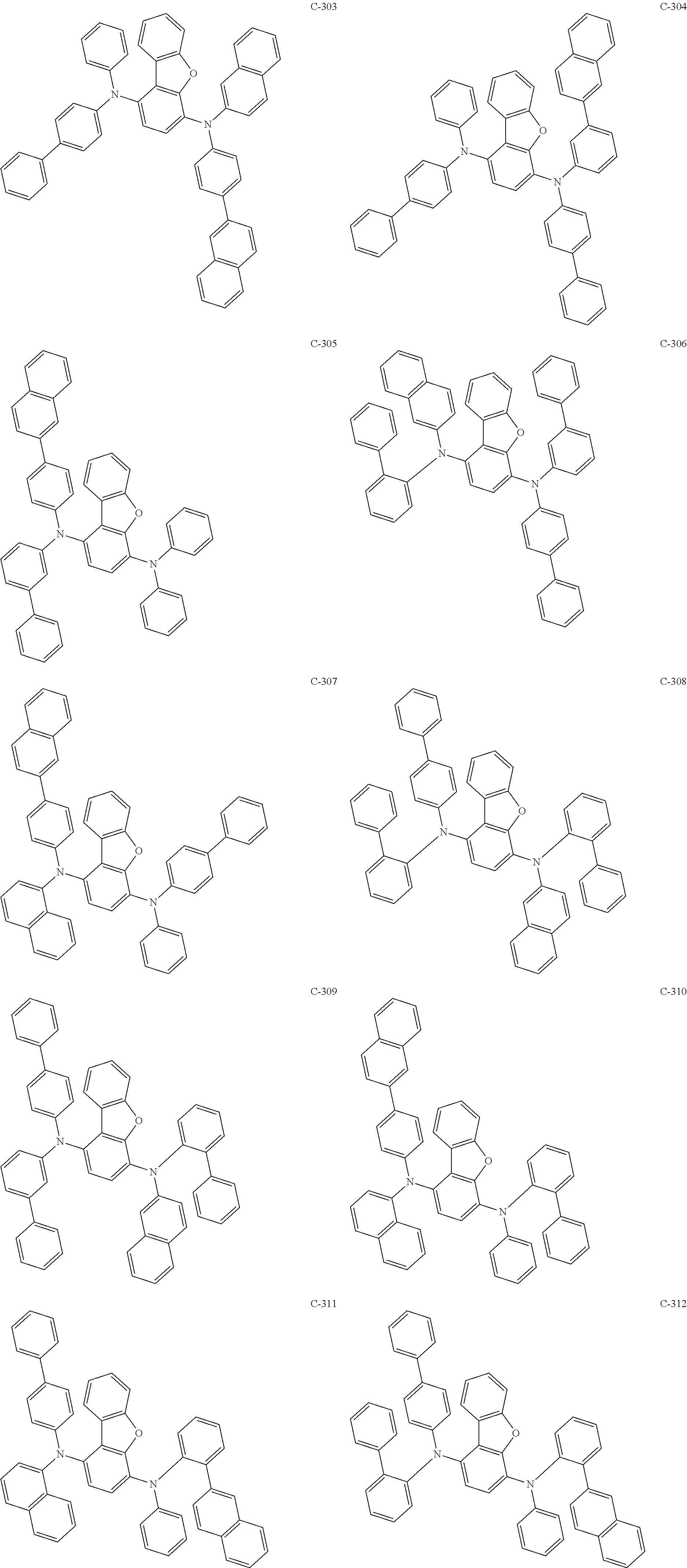

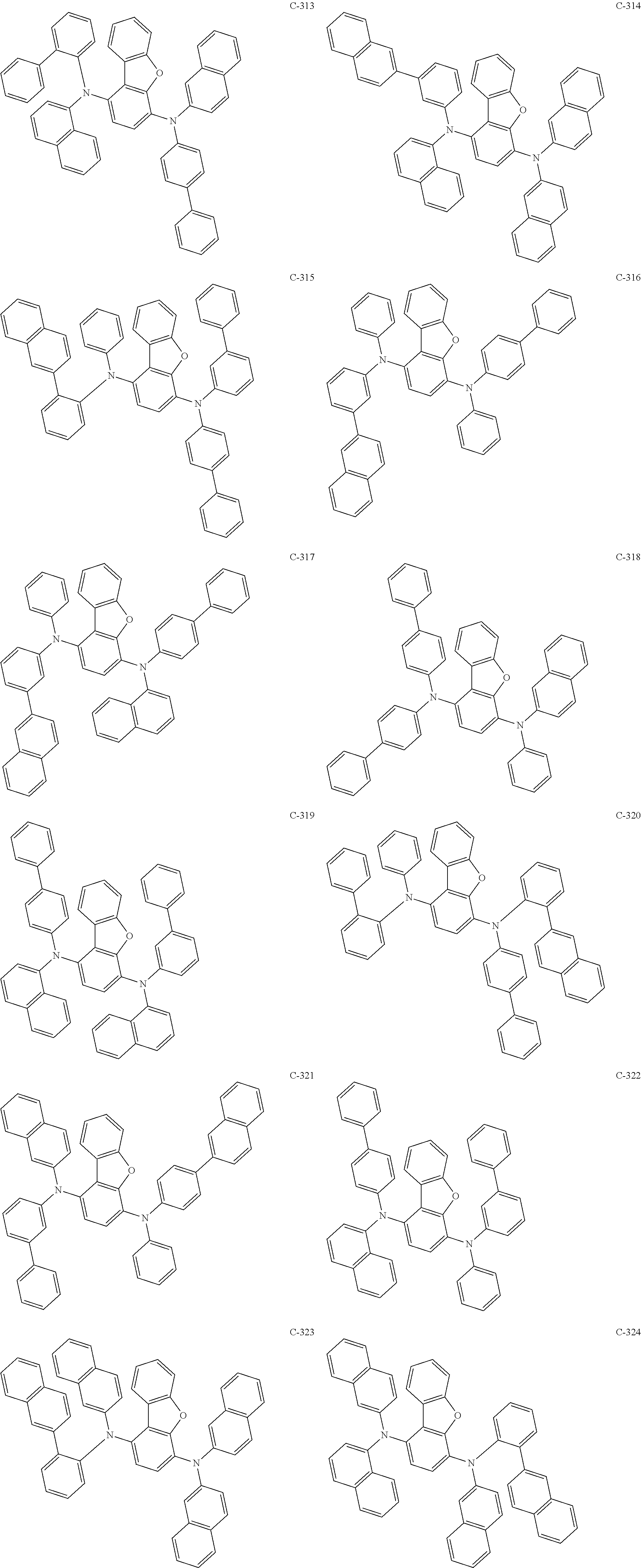

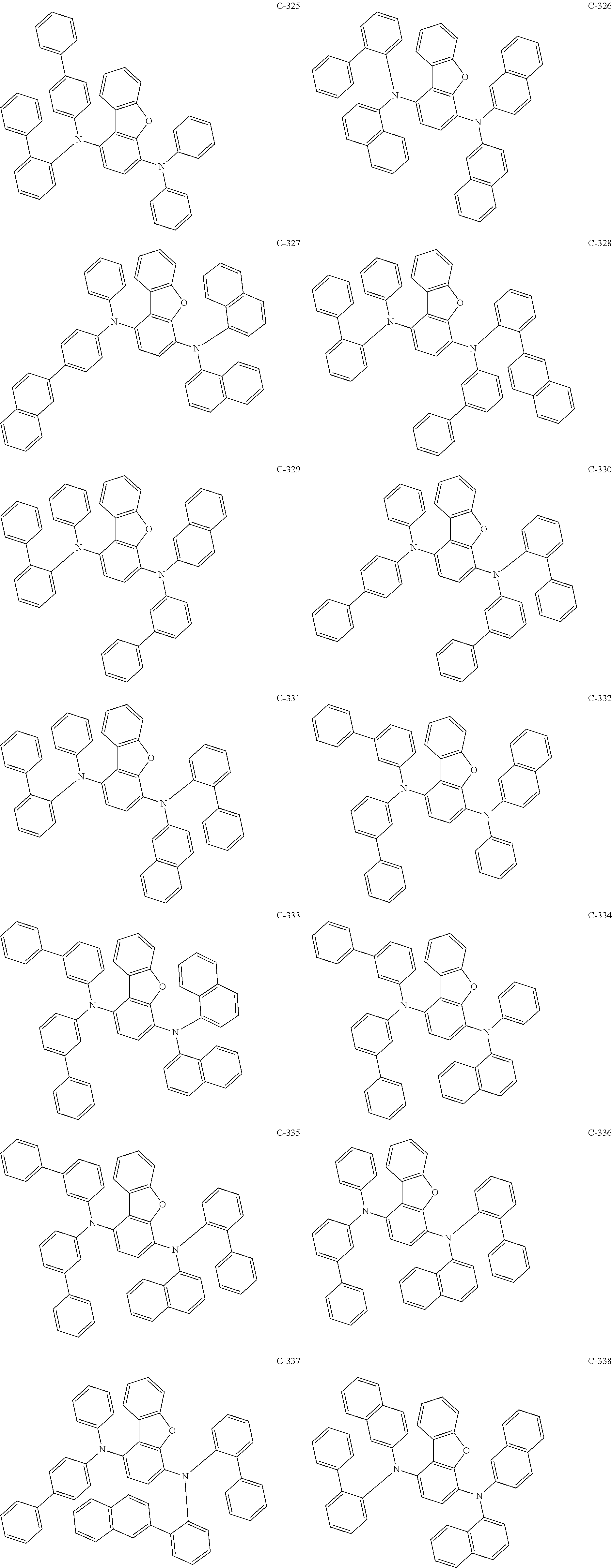

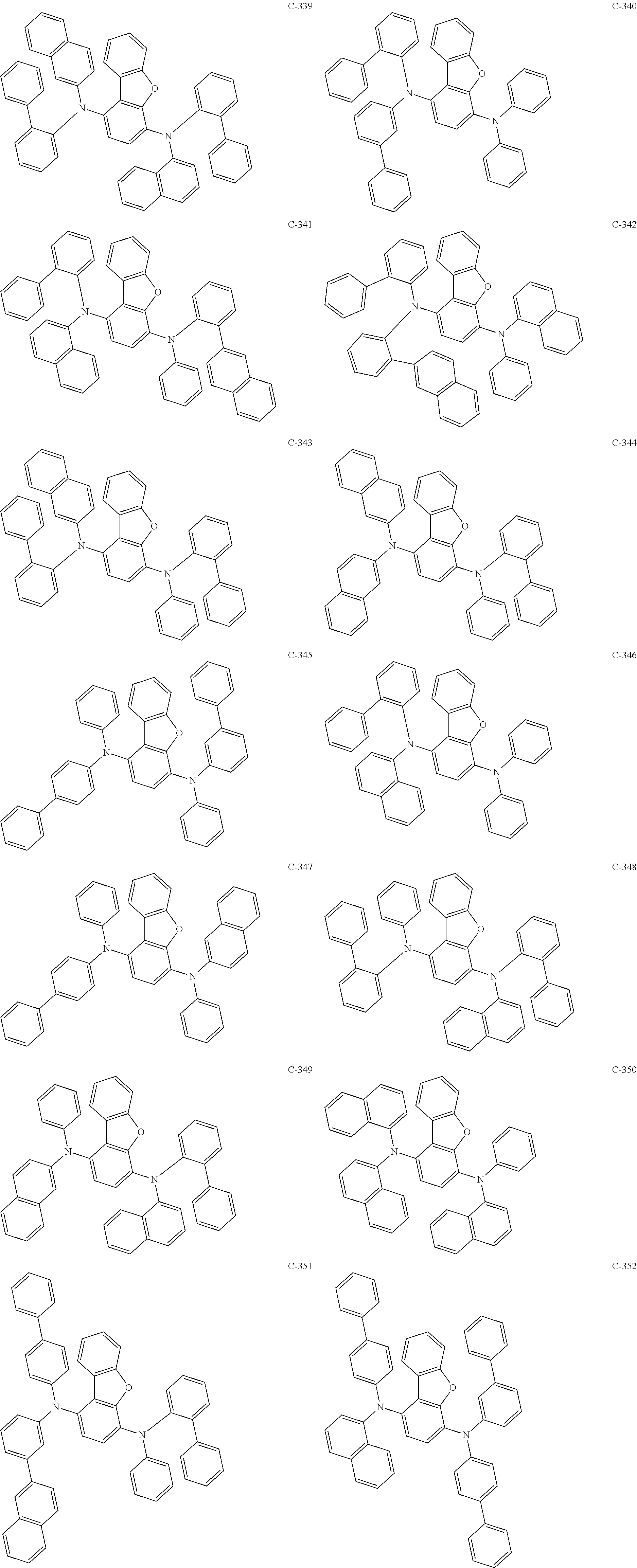

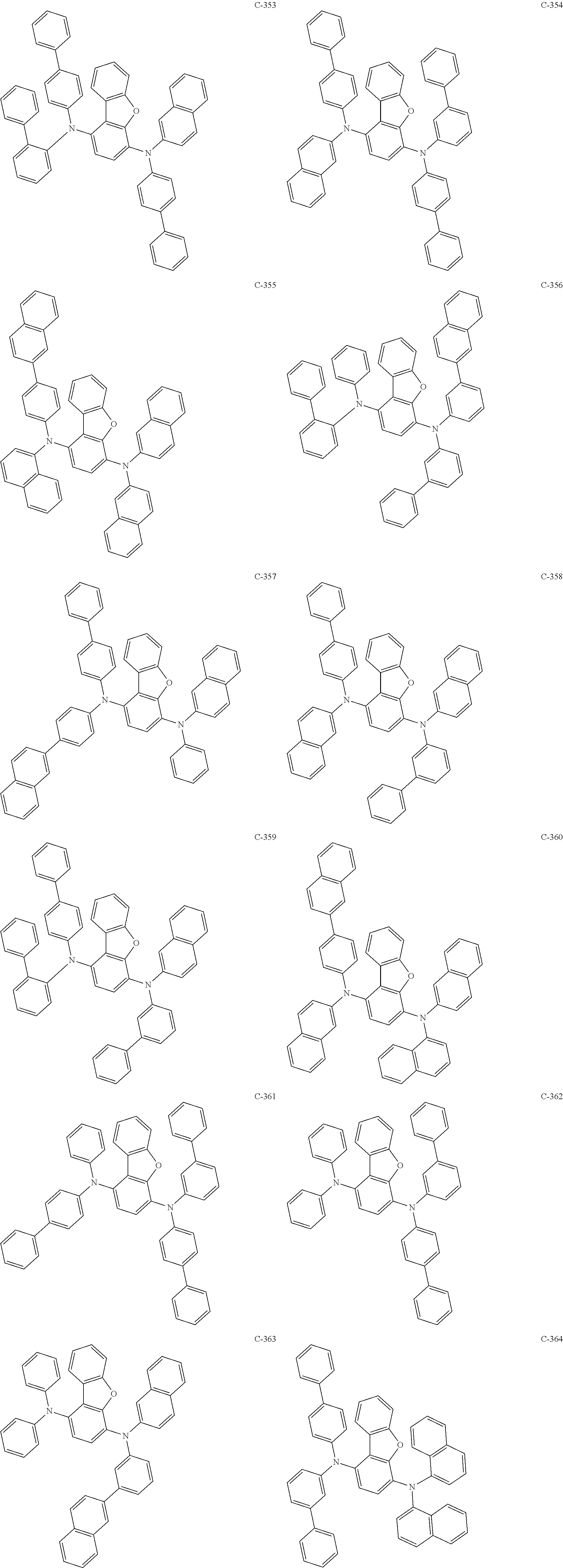

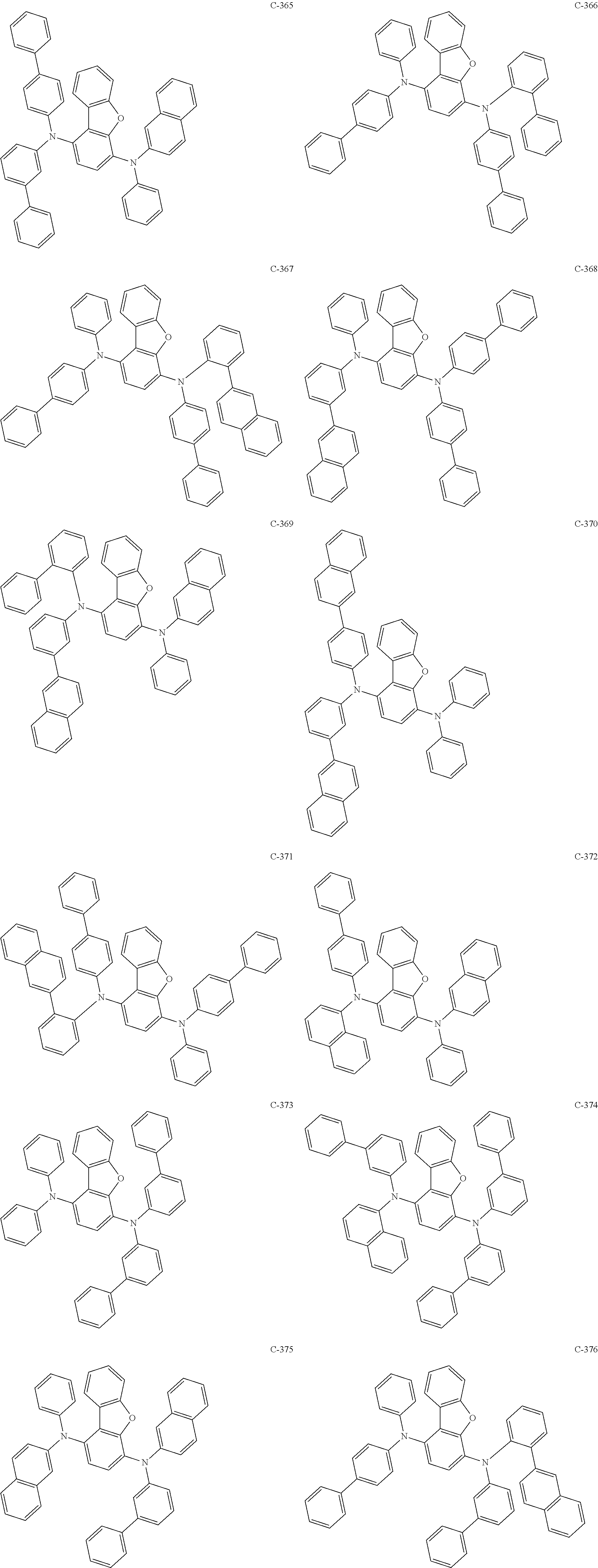

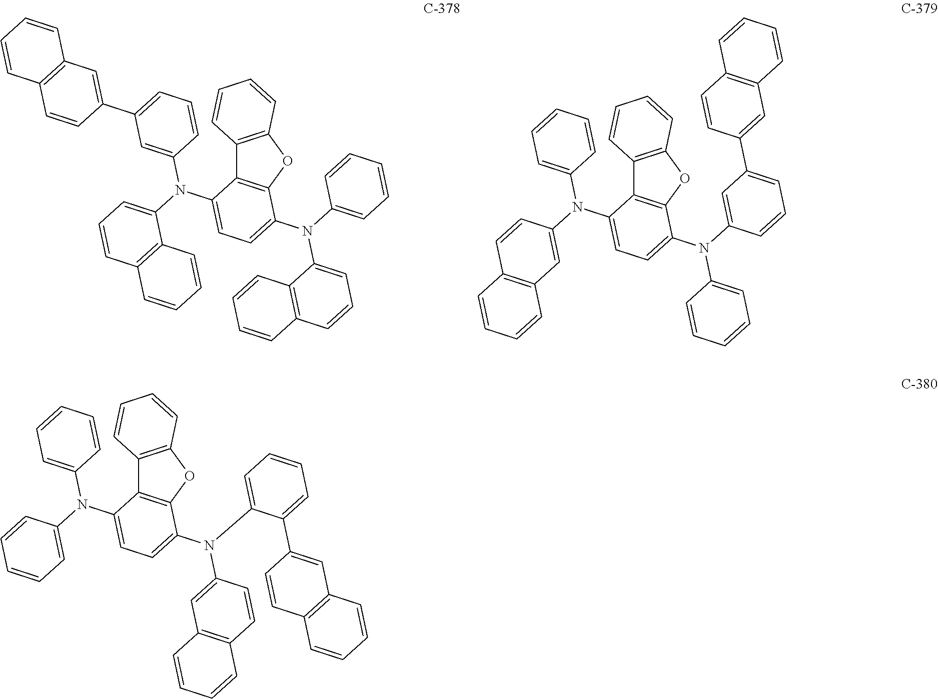 [SMILES: c1ccc(N(c2cccc3ccccc23)c2ccc(N(c3cccc(-c4ccc5ccccc5c4)c3)c3cccc4ccccc34)c3c2oc2ccccc23)cc1 ; c1ccc(N(c2cccc(-c3ccc4ccccc4c3)c2)c2ccc(N(c3ccccc3)c3ccc4ccccc4c3)c3c2oc2ccccc23)cc1 ; c1ccc(N(c2ccccc2)c2ccc(N(c3ccc4ccccc4c3)c3ccccc3-c3ccc4ccccc4c3)c3oc4ccccc4c23)cc1]

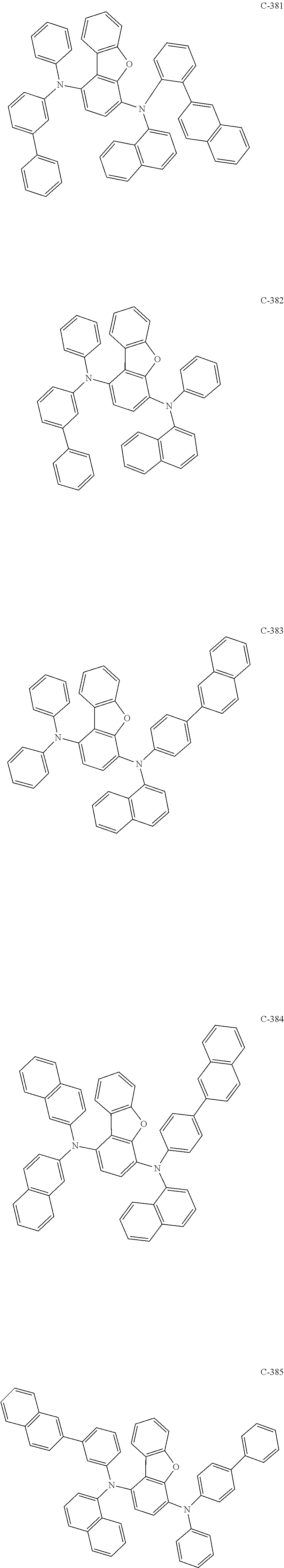 [SMILES: c1ccc(-c2cccc(N(c3ccccc3)c3ccc(N(c4ccccc4-c4ccc5ccccc5c4)c4cccc5ccccc45)c4oc5ccccc5c34)c2)cc1 ; c1ccc(-c2cccc(N(c3ccccc3)c3ccc(N(c4ccccc4)c4cccc5ccccc45)c4oc5ccccc5c34)c2)cc1 ; c1ccc(N(c2ccccc2)c2ccc(N(c3ccc(-c4ccc5ccccc5c4)cc3)c3cccc4ccccc34)c3oc4ccccc4c23)cc1 ; c1ccc2cc(-c3ccc(N(c4cccc5ccccc45)c4ccc(N(c5ccc6ccccc6c5)c5ccc6ccccc6c5)c5c4oc4ccccc45)cc3)ccc2c1 ; c1ccc(-c2ccc(N(c3ccccc3)c3ccc(N(c4cccc(-c5ccc6ccccc6c5)c4)c4cccc5ccccc45)c4c3oc3ccccc34)cc2)cc1]

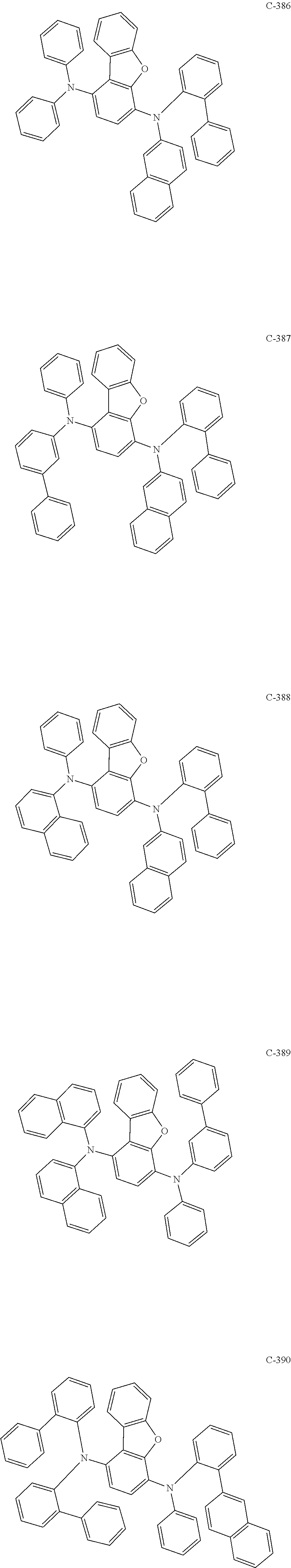 [SMILES: c1ccc(-c2ccccc2N(c2ccc3ccccc3c2)c2ccc(N(c3ccccc3)c3ccccc3)c3c2oc2ccccc23)cc1 ; c1ccc(-c2cccc(N(c3ccccc3)c3ccc(N(c4ccc5ccccc5c4)c4ccccc4-c4ccccc4)c4oc5ccccc5c34)c2)cc1 ; c1ccc(-c2ccccc2N(c2ccc3ccccc3c2)c2ccc(N(c3ccccc3)c3cccc4ccccc34)c3c2oc2ccccc23)cc1 ; c1ccc(-c2cccc(N(c3ccccc3)c3ccc(N(c4cccc5ccccc45)c4cccc5ccccc45)c4c3oc3ccccc34)c2)cc1 ; c1ccc(-c2ccccc2N(c2ccccc2-c2ccccc2)c2ccc(N(c3ccccc3)c3ccccc3-c3ccc4ccccc4c3)c3oc4ccccc4c23)cc1]

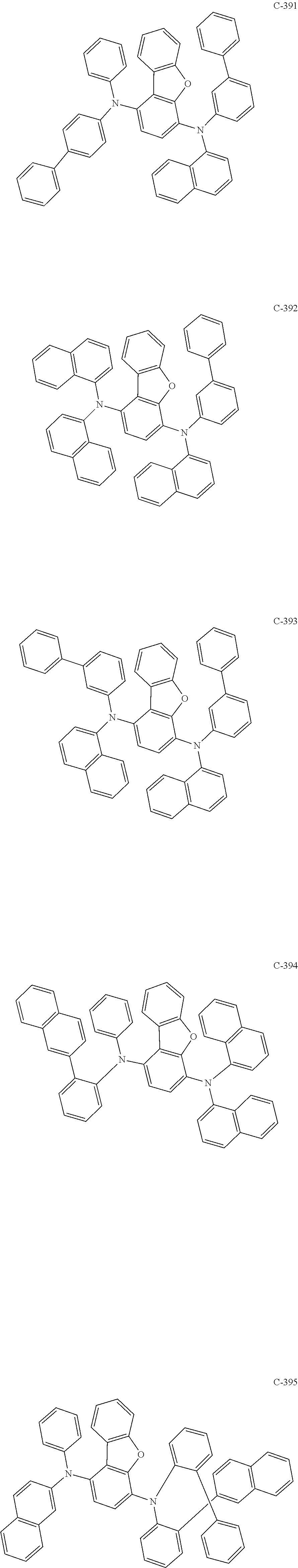 [SMILES: c1ccc(-c2ccc(N(c3ccccc3)c3ccc(N(c4cccc(-c5ccccc5)c4)c4cccc5ccccc45)c4oc5ccccc5c34)cc2)cc1 ; c1ccc(-c2cccc(N(c3cccc4ccccc34)c3ccc(N(c4cccc5ccccc45)c4cccc5ccccc45)c4c3oc3ccccc34)c2)cc1 ; c1ccc(-c2cccc(N(c3cccc4ccccc34)c3ccc(N(c4cccc(-c5ccccc5)c4)c4cccc5ccccc45)c4c3oc3ccccc34)c2)cc1 ; c1ccc(N(c2ccccc2-c2ccc3ccccc3c2)c2ccc(N(c3cccc4ccccc34)c3cccc4ccccc34)c3oc4ccccc4c23)cc1 ; c1ccc(-c2ccccc2N(c2ccccc2-c2ccc3ccccc3c2)c2ccc(N(c3ccccc3)c3ccc4ccccc4c3)c3c2oc2ccccc23)cc1]

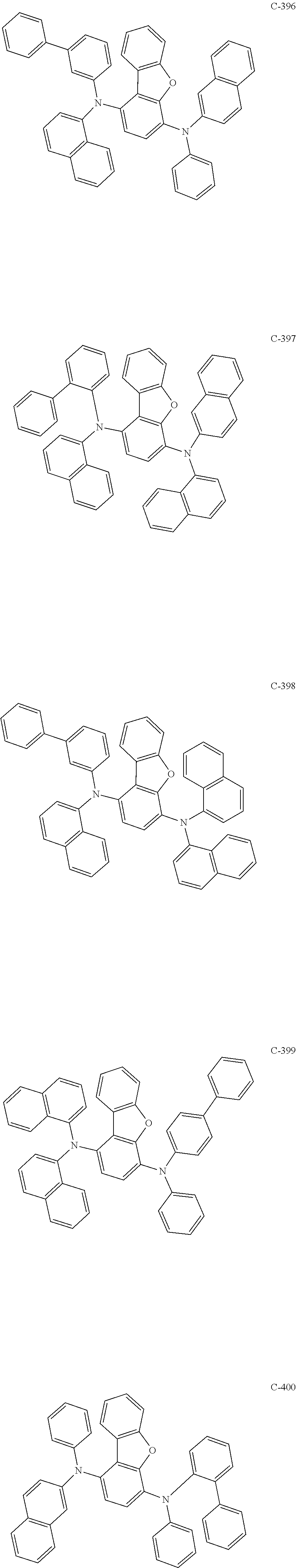 [SMILES: c1ccc(-c2cccc(N(c3cccc4ccccc34)c3ccc(N(c4ccccc4)c4ccc5ccccc5c4)c4oc5ccccc5c34)c2)cc1 ; c1ccc(-c2ccccc2N(c2cccc3ccccc23)c2ccc(N(c3ccc4ccccc4c3)c3cccc4ccccc34)c3oc4ccccc4c23)cc1 ; c1ccc(-c2cccc(N(c3cccc4ccccc34)c3ccc(N(c4cccc5ccccc45)c4cccc5ccccc45)c4oc5ccccc5c34)c2)cc1 ; c1ccc(-c2ccc(N(c3ccccc3)c3ccc(N(c4cccc5ccccc45)c4cccc5ccccc45)c4c3oc3ccccc34)cc2)cc1 ; c1ccc(-c2ccccc2N(c2ccccc2)c2ccc(N(c3ccccc3)c3ccc4ccccc4c3)c3c2oc2ccccc23)cc1]

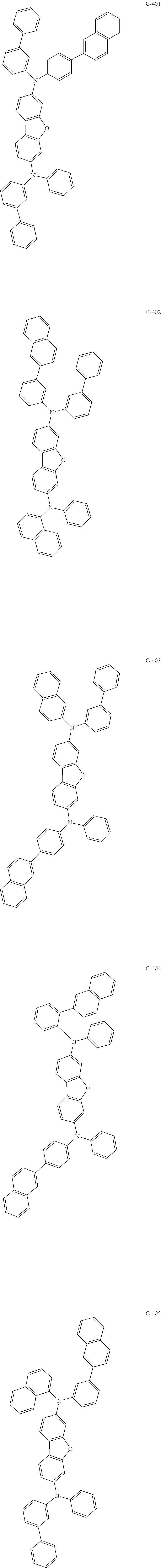 [SMILES: c1ccc(-c2cccc(N(c3ccccc3)c3ccc4c(c3)oc3cc(N(c5ccc(-c6ccc7ccccc7c6)cc5)c5cccc(-c6ccccc6)c5)ccc34)c2)cc1 ; c1ccc(-c2cccc(N(c3cccc(-c4ccc5ccccc5c4)c3)c3ccc4c(c3)oc3cc(N(c5ccccc5)c5cccc6ccccc56)ccc34)c2)cc1 ; c1ccc(-c2cccc(N(c3ccc4ccccc4c3)c3ccc4c(c3)oc3cc(N(c5ccccc5)c5ccc(-c6ccc7ccccc7c6)cc5)ccc34)c2)cc1 ; c1ccc(N(c2ccc(-c3ccc4ccccc4c3)cc2)c2ccc3c(c2)oc2cc(N(c4ccccc4)c4ccccc4-c4ccc5ccccc5c4)ccc23)cc1 ; c1ccc(-c2cccc(N(c3ccccc3)c3ccc4c(c3)oc3cc(N(c5cccc(-c6ccc7ccccc7c6)c5)c5cccc6ccccc56)ccc34)c2)cc1]

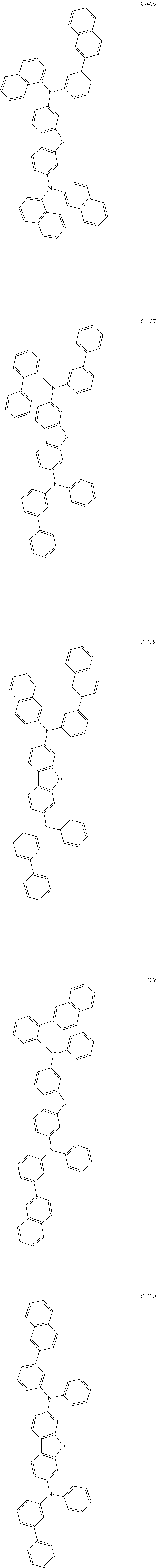 [SMILES: c1cc(-c2ccc3ccccc3c2)cc(N(c2ccc3c(c2)oc2cc(N(c4ccc5ccccc5c4)c4cccc5ccccc45)ccc23)c2cccc3ccccc23)c1 ; c1ccc(-c2cccc(N(c3ccccc3)c3ccc4c(c3)oc3cc(N(c5cccc(-c6ccccc6)c5)c5ccccc5-c5ccccc5)ccc34)c2)cc1 ; c1ccc(-c2cccc(N(c3ccccc3)c3ccc4c(c3)oc3cc(N(c5cccc(-c6ccc7ccccc7c6)c5)c5ccc6ccccc6c5)ccc34)c2)cc1 ; c1ccc(N(c2cccc(-c3ccc4ccccc4c3)c2)c2ccc3c(c2)oc2cc(N(c4ccccc4)c4ccccc4-c4ccc5ccccc5c4)ccc23)cc1 ; c1ccc(-c2cccc(N(c3ccccc3)c3ccc4c(c3)oc3cc(N(c5ccccc5)c5cccc(-c6ccc7ccccc7c6)c5)ccc34)c2)cc1]

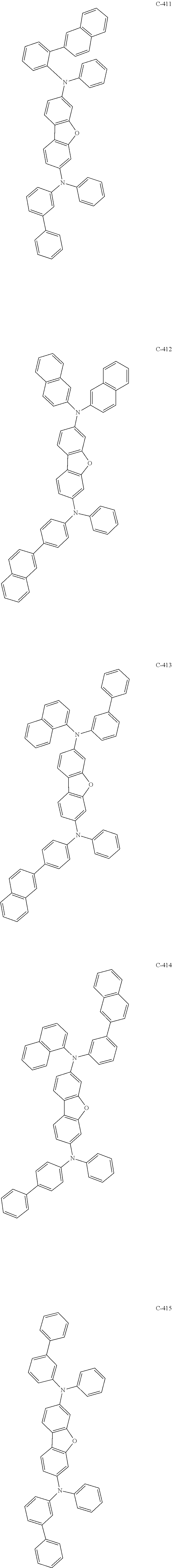 [SMILES: c1ccc(-c2cccc(N(c3ccccc3)c3ccc4c(c3)oc3cc(N(c5ccccc5)c5ccccc5-c5ccc6ccccc6c5)ccc34)c2)cc1 ; c1ccc(N(c2ccc(-c3ccc4ccccc4c3)cc2)c2ccc3c(c2)oc2cc(N(c4ccc5ccccc5c4)c4ccc5ccccc5c4)ccc23)cc1 ; c1ccc(-c2cccc(N(c3ccc4c(c3)oc3cc(N(c5ccccc5)c5ccc(-c6ccc7ccccc7c6)cc5)ccc34)c3cccc4ccccc34)c2)cc1 ; c1ccc(-c2ccc(N(c3ccccc3)c3ccc4c(c3)oc3cc(N(c5cccc(-c6ccc7ccccc7c6)c5)c5cccc6ccccc56)ccc34)cc2)cc1 ; c1ccc(-c2cccc(N(c3ccccc3)c3ccc4c(c3)oc3cc(N(c5ccccc5)c5cccc(-c6ccccc6)c5)ccc34)c2)cc1]

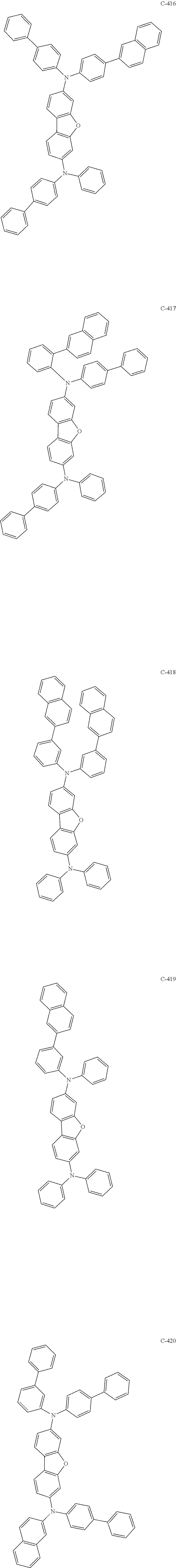 [SMILES: c1ccc(-c2ccc(N(c3ccccc3)c3ccc4c(c3)oc3cc(N(c5ccc(-c6ccccc6)cc5)c5ccc(-c6ccc7ccccc7c6)cc5)ccc34)cc2)cc1 ; c1ccc(-c2ccc(N(c3ccccc3)c3ccc4c(c3)oc3cc(N(c5ccc(-c6ccccc6)cc5)c5ccccc5-c5ccc6ccccc6c5)ccc34)cc2)cc1 ; c1ccc(N(c2ccccc2)c2ccc3c(c2)oc2cc(N(c4cccc(-c5ccc6ccccc6c5)c4)c4cccc(-c5ccc6ccccc6c5)c4)ccc23)cc1 ; c1ccc(N(c2ccccc2)c2ccc3c(c2)oc2cc(N(c4ccccc4)c4cccc(-c5ccc6ccccc6c5)c4)ccc23)cc1 ; c1ccc(-c2ccc(N(c3cccc(-c4ccccc4)c3)c3ccc4c(c3)oc3cc(N(c5ccc(-c6ccccc6)cc5)c5ccc6ccccc6c5)ccc34)cc2)cc1]

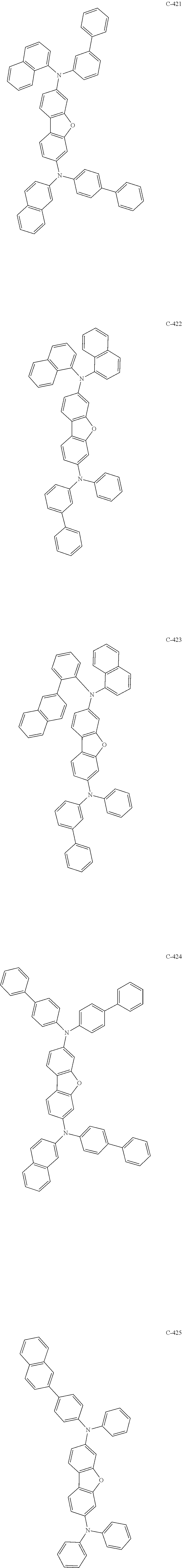 [SMILES: c1ccc(-c2ccc(N(c3ccc4ccccc4c3)c3ccc4c(c3)oc3cc(N(c5cccc(-c6ccccc6)c5)c5cccc6ccccc56)ccc34)cc2)cc1 ; c1ccc(-c2cccc(N(c3ccccc3)c3ccc4c(c3)oc3cc(N(c5cccc6ccccc56)c5cccc6ccccc56)ccc34)c2)cc1 ; c1ccc(-c2cccc(N(c3ccccc3)c3ccc4c(c3)oc3cc(N(c5ccccc5-c5ccc6ccccc6c5)c5cccc6ccccc56)ccc34)c2)cc1 ; c1ccc(-c2ccc(N(c3ccc(-c4ccccc4)cc3)c3ccc4c(c3)oc3cc(N(c5ccc(-c6ccccc6)cc5)c5ccc6ccccc6c5)ccc34)cc2)cc1 ; c1ccc(N(c2ccccc2)c2ccc3c(c2)oc2cc(N(c4ccccc4)c4ccc(-c5ccc6ccccc6c5)cc4)ccc23)cc1]

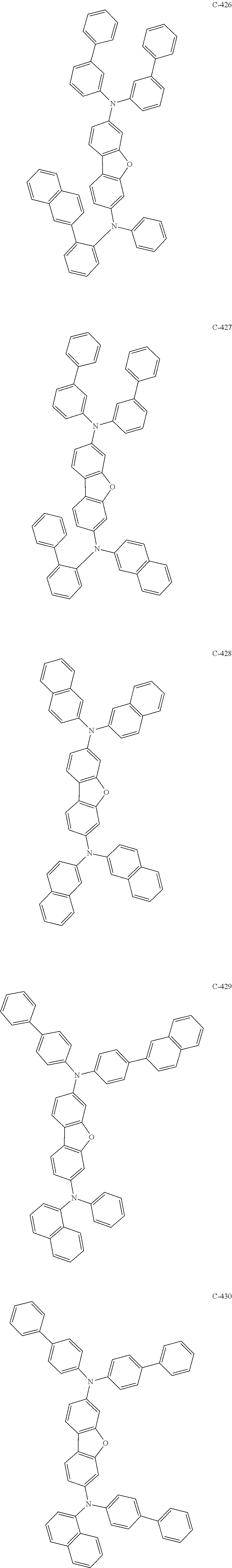 [SMILES: c1ccc(-c2cccc(N(c3cccc(-c4ccccc4)c3)c3ccc4c(c3)oc3cc(N(c5ccccc5)c5ccccc5-c5ccc6ccccc6c5)ccc34)c2)cc1 ; c1ccc(-c2cccc(N(c3cccc(-c4ccccc4)c3)c3ccc4c(c3)oc3cc(N(c5ccc6ccccc6c5)c5ccccc5-c5ccccc5)ccc34)c2)cc1 ; c1ccc2cc(N(c3ccc4ccccc4c3)c3ccc4c(c3)oc3cc(N(c5ccc6ccccc6c5)c5ccc6ccccc6c5)ccc34)ccc2c1 ; c1ccc(-c2ccc(N(c3ccc(-c4ccc5ccccc5c4)cc3)c3ccc4c(c3)oc3cc(N(c5ccccc5)c5cccc6ccccc56)ccc34)cc2)cc1 ; c1ccc(-c2ccc(N(c3ccc(-c4ccccc4)cc3)c3ccc4c(c3)oc3cc(N(c5ccc(-c6ccccc6)cc5)c5cccc6ccccc56)ccc34)cc2)cc1]

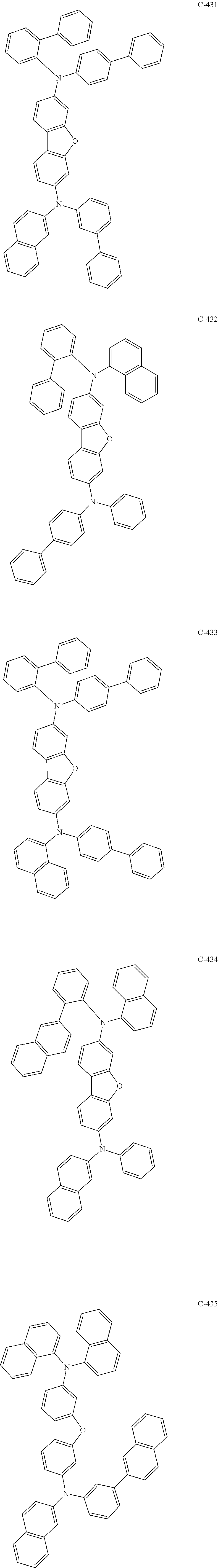 [SMILES: c1ccc(-c2ccc(N(c3ccc4c(c3)oc3cc(N(c5cccc(-c6ccccc6)c5)c5ccc6ccccc6c5)ccc34)c3ccccc3-c3ccccc3)cc2)cc1 ; c1ccc(-c2ccc(N(c3ccccc3)c3ccc4c(c3)oc3cc(N(c5ccccc5-c5ccccc5)c5cccc6ccccc56)ccc34)cc2)cc1 ; c1ccc(-c2ccc(N(c3ccc4c(c3)oc3cc(N(c5ccc(-c6ccccc6)cc5)c5cccc6ccccc56)ccc34)c3ccccc3-c3ccccc3)cc2)cc1 ; c1ccc(N(c2ccc3ccccc3c2)c2ccc3c(c2)oc2cc(N(c4ccccc4-c4ccc5ccccc5c4)c4cccc5ccccc45)ccc23)cc1 ; c1cc(-c2ccc3ccccc3c2)cc(N(c2ccc3ccccc3c2)c2ccc3c(c2)oc2cc(N(c4cccc5ccccc45)c4cccc5ccccc45)ccc23)c1]

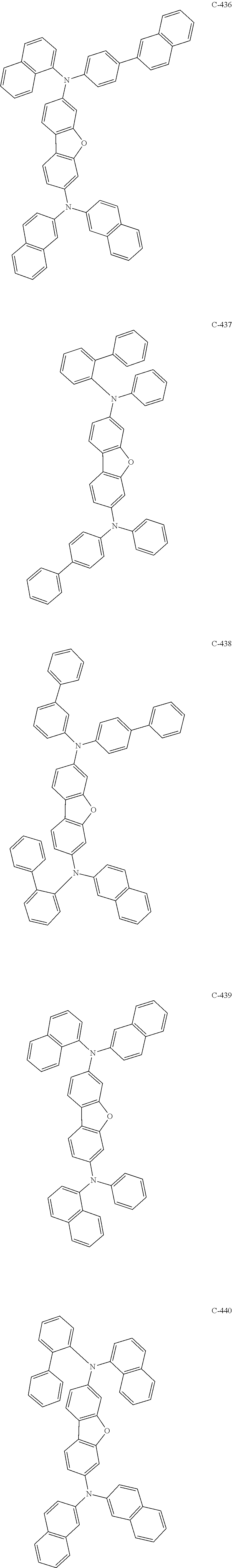 [SMILES: c1ccc2cc(-c3ccc(N(c4ccc5c(c4)oc4cc(N(c6ccc7ccccc7c6)c6ccc7ccccc7c6)ccc45)c4cccc5ccccc45)cc3)ccc2c1 ; c1ccc(-c2ccc(N(c3ccccc3)c3ccc4c(c3)oc3cc(N(c5ccccc5)c5ccccc5-c5ccccc5)ccc34)cc2)cc1 ; c1ccc(-c2ccc(N(c3cccc(-c4ccccc4)c3)c3ccc4c(c3)oc3cc(N(c5ccc6ccccc6c5)c5ccccc5-c5ccccc5)ccc34)cc2)cc1 ; c1ccc(N(c2ccc3c(c2)oc2cc(N(c4ccc5ccccc5c4)c4cccc5ccccc45)ccc23)c2cccc3ccccc23)cc1 ; c1ccc(-c2ccccc2N(c2ccc3c(c2)oc2cc(N(c4ccc5ccccc5c4)c4ccc5ccccc5c4)ccc23)c2cccc3ccccc23)cc1]

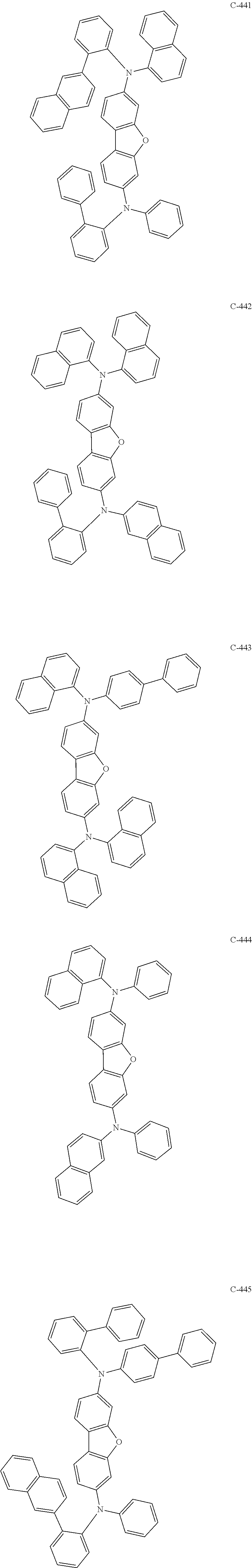 [SMILES: c1ccc(-c2ccccc2N(c2ccccc2)c2ccc3c(c2)oc2cc(N(c4ccccc4-c4ccc5ccccc5c4)c4cccc5ccccc45)ccc23)cc1 ; c1ccc(-c2ccccc2N(c2ccc3ccccc3c2)c2ccc3c(c2)oc2cc(N(c4cccc5ccccc45)c4cccc5ccccc45)ccc23)cc1 ; c1ccc(-c2ccc(N(c3ccc4c(c3)oc3cc(N(c5cccc6ccccc56)c5cccc6ccccc56)ccc34)c3cccc4ccccc34)cc2)cc1 ; c1ccc(N(c2ccc3ccccc3c2)c2ccc3c(c2)oc2cc(N(c4ccccc4)c4cccc5ccccc45)ccc23)cc1 ; c1ccc(-c2ccc(N(c3ccc4c(c3)oc3cc(N(c5ccccc5)c5ccccc5-c5ccc6ccccc6c5)ccc34)c3ccccc3-c3ccccc3)cc2)cc1]

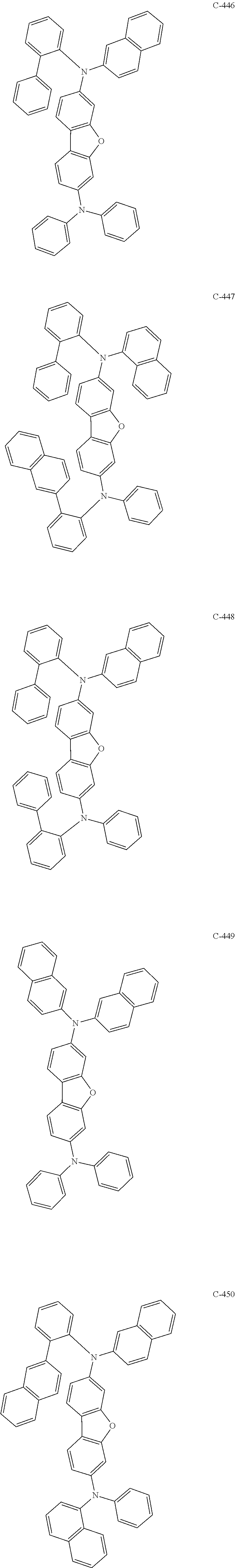 [SMILES: c1ccc(-c2ccccc2N(c2ccc3ccccc3c2)c2ccc3c(c2)oc2cc(N(c4ccccc4)c4ccccc4)ccc23)cc1 ; c1ccc(-c2ccccc2N(c2ccc3c(c2)oc2cc(N(c4ccccc4)c4ccccc4-c4ccc5ccccc5c4)ccc23)c2cccc3ccccc23)cc1 ; c1ccc(-c2ccccc2N(c2ccccc2)c2ccc3c(c2)oc2cc(N(c4ccc5ccccc5c4)c4ccccc4-c4ccccc4)ccc23)cc1 ; c1ccc(N(c2ccccc2)c2ccc3c(c2)oc2cc(N(c4ccc5ccccc5c4)c4ccc5ccccc5c4)ccc23)cc1 ; c1ccc(N(c2ccc3c(c2)oc2cc(N(c4ccc5ccccc5c4)c4ccccc4-c4ccc5ccccc5c4)ccc23)c2cccc3ccccc23)cc1]

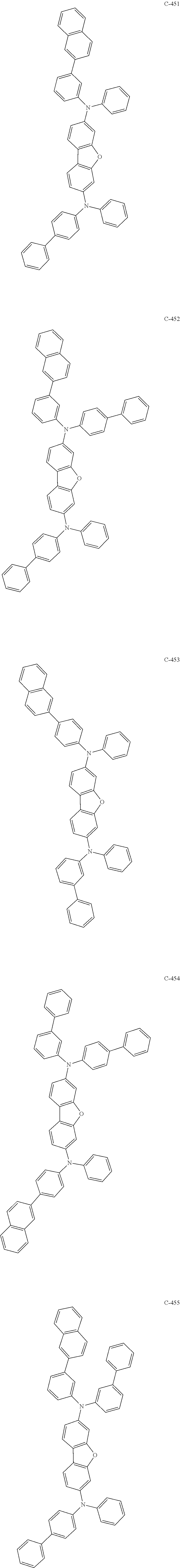 [SMILES: c1ccc(-c2ccc(N(c3ccccc3)c3ccc4c(c3)oc3cc(N(c5ccccc5)c5cccc(-c6ccc7ccccc7c6)c5)ccc34)cc2)cc1 ; c1ccc(-c2ccc(N(c3ccccc3)c3ccc4c(c3)oc3cc(N(c5ccc(-c6ccccc6)cc5)c5cccc(-c6ccc7ccccc7c6)c5)ccc34)cc2)cc1 ; c1ccc(-c2cccc(N(c3ccccc3)c3ccc4c(c3)oc3cc(N(c5ccccc5)c5ccc(-c6ccc7ccccc7c6)cc5)ccc34)c2)cc1 ; c1ccc(-c2ccc(N(c3cccc(-c4ccccc4)c3)c3ccc4c(c3)oc3cc(N(c5ccccc5)c5ccc(-c6ccc7ccccc7c6)cc5)ccc34)cc2)cc1 ; c1ccc(-c2ccc(N(c3ccccc3)c3ccc4c(c3)oc3cc(N(c5cccc(-c6ccccc6)c5)c5cccc(-c6ccc7ccccc7c6)c5)ccc34)cc2)cc1]

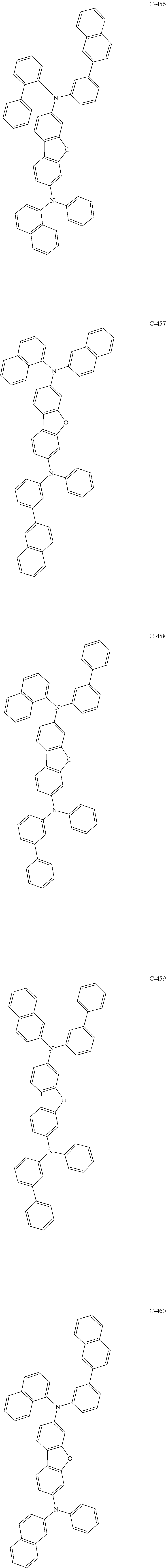 [SMILES: c1ccc(-c2ccccc2N(c2cccc(-c3ccc4ccccc4c3)c2)c2ccc3c(c2)oc2cc(N(c4ccccc4)c4cccc5ccccc45)ccc23)cc1 ; c1ccc(N(c2cccc(-c3ccc4ccccc4c3)c2)c2ccc3c(c2)oc2cc(N(c4ccc5ccccc5c4)c4cccc5ccccc45)ccc23)cc1 ; c1ccc(-c2cccc(N(c3ccccc3)c3ccc4c(c3)oc3cc(N(c5cccc(-c6ccccc6)c5)c5cccc6ccccc56)ccc34)c2)cc1 ; c1ccc(-c2cccc(N(c3ccccc3)c3ccc4c(c3)oc3cc(N(c5cccc(-c6ccccc6)c5)c5ccc6ccccc6c5)ccc34)c2)cc1 ; c1ccc(N(c2ccc3ccccc3c2)c2ccc3c(c2)oc2cc(N(c4cccc(-c5ccc6ccccc6c5)c4)c4cccc5ccccc45)ccc23)cc1]

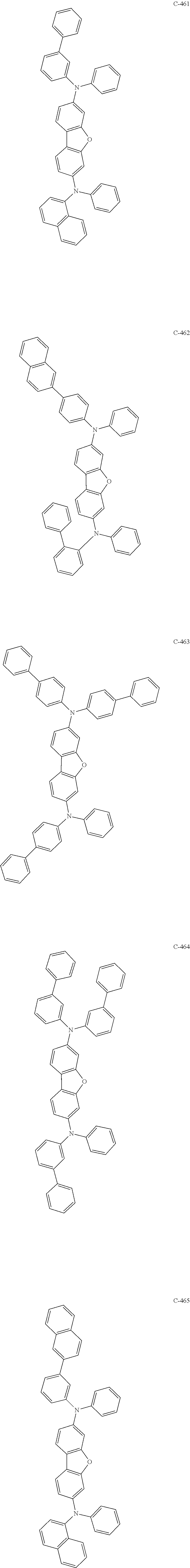 [SMILES: c1ccc(-c2cccc(N(c3ccccc3)c3ccc4c(c3)oc3cc(N(c5ccccc5)c5cccc6ccccc56)ccc34)c2)cc1 ; c1ccc(-c2ccccc2N(c2ccccc2)c2ccc3c(c2)oc2cc(N(c4ccccc4)c4ccc(-c5ccc6ccccc6c5)cc4)ccc23)cc1 ; c1ccc(-c2ccc(N(c3ccccc3)c3ccc4c(c3)oc3cc(N(c5ccc(-c6ccccc6)cc5)c5ccc(-c6ccccc6)cc5)ccc34)cc2)cc1 ; c1ccc(-c2cccc(N(c3ccccc3)c3ccc4c(c3)oc3cc(N(c5cccc(-c6ccccc6)c5)c5cccc(-c6ccccc6)c5)ccc34)c2)cc1 ; c1ccc(N(c2cccc(-c3ccc4ccccc4c3)c2)c2ccc3c(c2)oc2cc(N(c4ccccc4)c4cccc5ccccc45)ccc23)cc1]

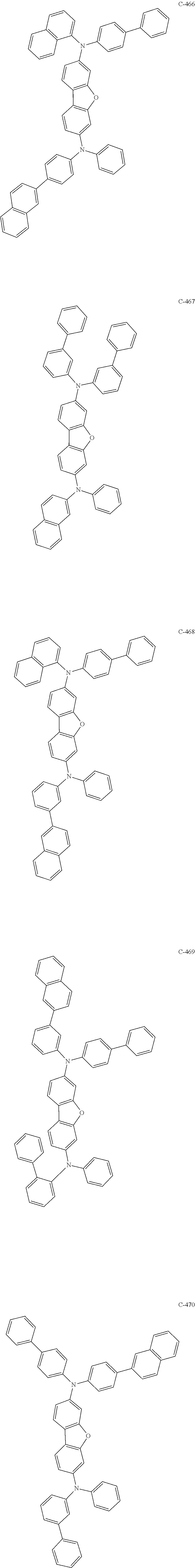 [SMILES: c1ccc(-c2ccc(N(c3ccc4c(c3)oc3cc(N(c5ccccc5)c5ccc(-c6ccc7ccccc7c6)cc5)ccc34)c3cccc4ccccc34)cc2)cc1 ; c1ccc(-c2cccc(N(c3cccc(-c4ccccc4)c3)c3ccc4c(c3)oc3cc(N(c5ccccc5)c5ccc6ccccc6c5)ccc34)c2)cc1 ; c1ccc(-c2ccc(N(c3ccc4c(c3)oc3cc(N(c5ccccc5)c5cccc(-c6ccc7ccccc7c6)c5)ccc34)c3cccc4ccccc34)cc2)cc1 ; c1ccc(-c2ccc(N(c3cccc(-c4ccc5ccccc5c4)c3)c3ccc4c(c3)oc3cc(N(c5ccccc5)c5ccccc5-c5ccccc5)ccc34)cc2)cc1 ; c1ccc(-c2ccc(N(c3ccc(-c4ccc5ccccc5c4)cc3)c3ccc4c(c3)oc3cc(N(c5ccccc5)c5cccc(-c6ccccc6)c5)ccc34)cc2)cc1]

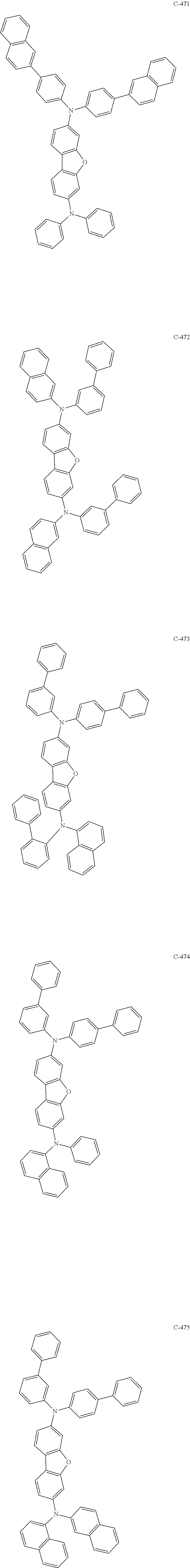 [SMILES: c1ccc(N(c2ccccc2)c2ccc3c(c2)oc2cc(N(c4ccc(-c5ccc6ccccc6c5)cc4)c4ccc(-c5ccc6ccccc6c5)cc4)ccc23)cc1 ; c1ccc(-c2cccc(N(c3ccc4ccccc4c3)c3ccc4c(c3)oc3cc(N(c5cccc(-c6ccccc6)c5)c5ccc6ccccc6c5)ccc34)c2)cc1 ; c1ccc(-c2ccc(N(c3cccc(-c4ccccc4)c3)c3ccc4c(c3)oc3cc(N(c5ccccc5-c5ccccc5)c5cccc6ccccc56)ccc34)cc2)cc1 ; c1ccc(-c2ccc(N(c3cccc(-c4ccccc4)c3)c3ccc4c(c3)oc3cc(N(c5ccccc5)c5cccc6ccccc56)ccc34)cc2)cc1 ; c1ccc(-c2ccc(N(c3cccc(-c4ccccc4)c3)c3ccc4c(c3)oc3cc(N(c5ccc6ccccc6c5)c5cccc6ccccc56)ccc34)cc2)cc1]

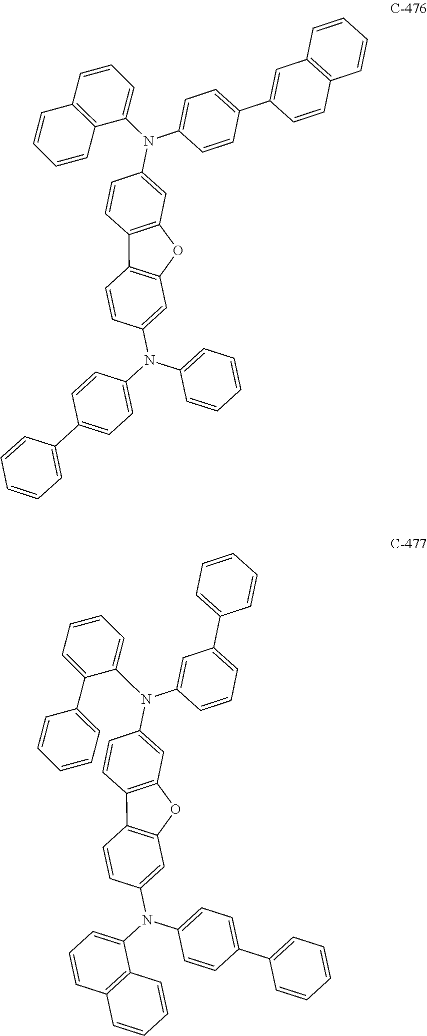 [SMILES: c1ccc(-c2ccc(N(c3ccccc3)c3ccc4c(c3)oc3cc(N(c5ccc(-c6ccc7ccccc7c6)cc5)c5cccc6ccccc56)ccc34)cc2)cc1 ; c1ccc(-c2ccc(N(c3ccc4c(c3)oc3cc(N(c5cccc(-c6ccccc6)c5)c5ccccc5-c5ccccc5)ccc34)c3cccc4ccccc34)cc2)cc1]

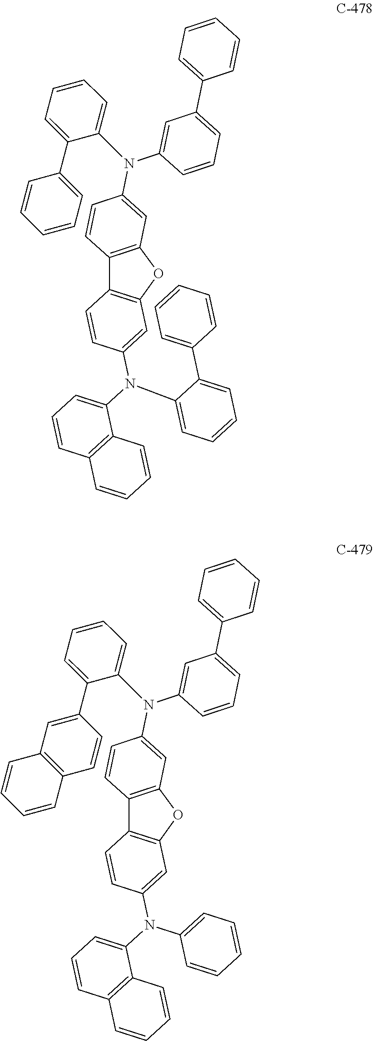 [SMILES: c1ccc(-c2cccc(N(c3ccc4c(c3)oc3cc(N(c5ccccc5-c5ccccc5)c5cccc6ccccc56)ccc34)c3ccccc3-c3ccccc3)c2)cc1 ; c1ccc(-c2cccc(N(c3ccc4c(c3)oc3cc(N(c5ccccc5)c5cccc6ccccc56)ccc34)c3ccccc3-c3ccc4ccccc4c3)c2)cc1]

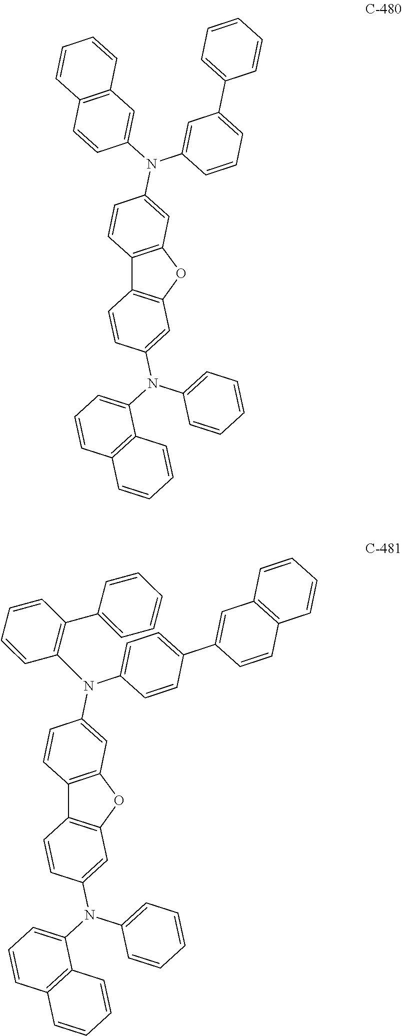 [SMILES: c1ccc(-c2cccc(N(c3ccc4ccccc4c3)c3ccc4c(c3)oc3cc(N(c5ccccc5)c5cccc6ccccc56)ccc34)c2)cc1 ; c1ccc(-c2ccccc2N(c2ccc(-c3ccc4ccccc4c3)cc2)c2ccc3c(c2)oc2cc(N(c4ccccc4)c4cccc5ccccc45)ccc23)cc1]

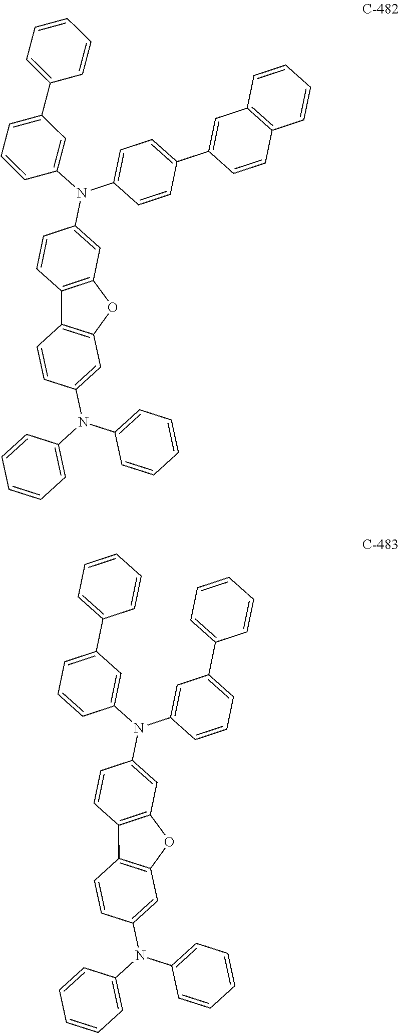 [SMILES: c1ccc(-c2cccc(N(c3ccc(-c4ccc5ccccc5c4)cc3)c3ccc4c(c3)oc3cc(N(c5ccccc5)c5ccccc5)ccc34)c2)cc1 ; c1ccc(-c2cccc(N(c3cccc(-c4ccccc4)c3)c3ccc4c(c3)oc3cc(N(c5ccccc5)c5ccccc5)ccc34)c2)cc1]

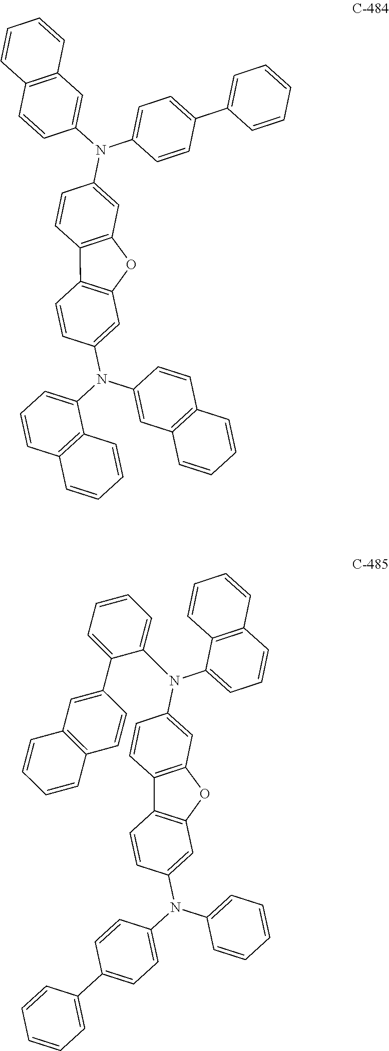 [SMILES: c1ccc(-c2ccc(N(c3ccc4ccccc4c3)c3ccc4c(c3)oc3cc(N(c5ccc6ccccc6c5)c5cccc6ccccc56)ccc34)cc2)cc1 ; c1ccc(-c2ccc(N(c3ccccc3)c3ccc4c(c3)oc3cc(N(c5ccccc5-c5ccc6ccccc6c5)c5cccc6ccccc56)ccc34)cc2)cc1]

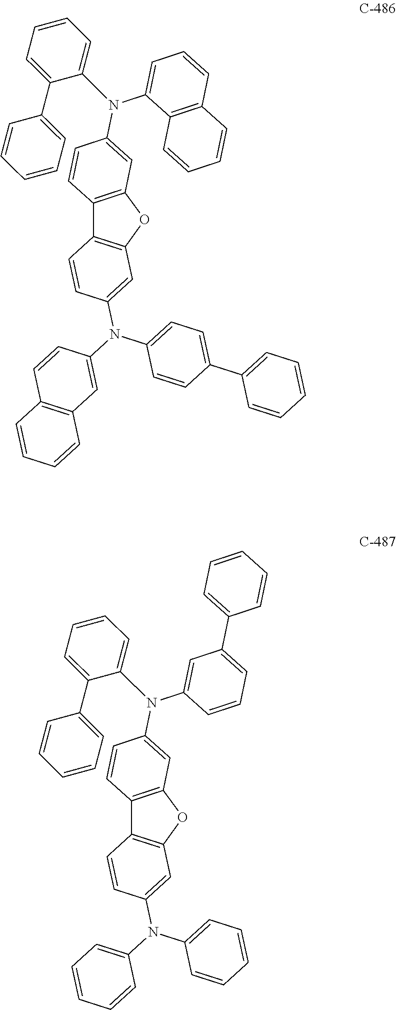 [SMILES: c1ccc(-c2ccc(N(c3ccc4ccccc4c3)c3ccc4c(c3)oc3cc(N(c5ccccc5-c5ccccc5)c5cccc6ccccc56)ccc34)cc2)cc1 ; c1ccc(-c2cccc(N(c3ccc4c(c3)oc3cc(N(c5ccccc5)c5ccccc5)ccc34)c3ccccc3-c3ccccc3)c2)cc1]

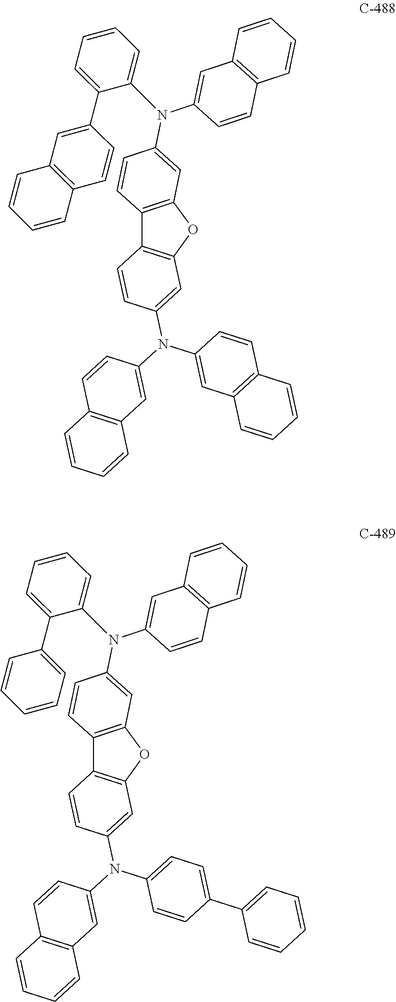 [SMILES: c1ccc(N(c2ccc3ccccc3c2)c2ccc3c(c2)oc2cc(N(c4ccc5ccccc5c4)c4ccc5ccccc5c4)ccc23)c(-c2ccc3ccccc3c2)c1 ; c1ccc(-c2ccc(N(c3ccc4ccccc4c3)c3ccc4c(c3)oc3cc(N(c5ccc6ccccc6c5)c5ccccc5-c5ccccc5)ccc34)cc2)cc1]

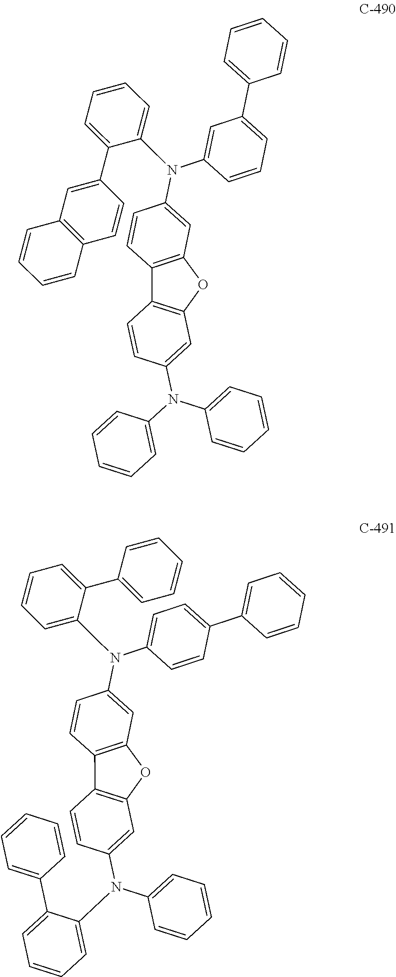 [SMILES: c1ccc(-c2cccc(N(c3ccc4c(c3)oc3cc(N(c5ccccc5)c5ccccc5)ccc34)c3ccccc3-c3ccc4ccccc4c3)c2)cc1 ; c1ccc(-c2ccc(N(c3ccc4c(c3)oc3cc(N(c5ccccc5)c5ccccc5-c5ccccc5)ccc34)c3ccccc3-c3ccccc3)cc2)cc1]

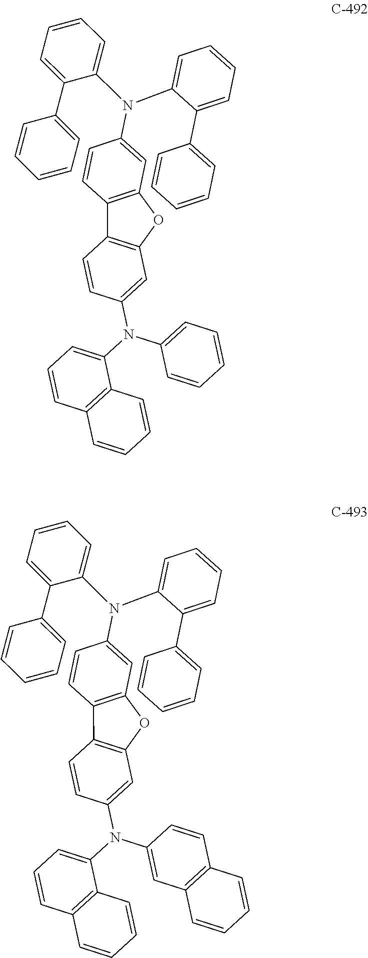 [SMILES: c1ccc(-c2ccccc2N(c2ccc3c(c2)oc2cc(N(c4ccccc4)c4cccc5ccccc45)ccc23)c2ccccc2-c2ccccc2)cc1 ; c1ccc(-c2ccccc2N(c2ccc3c(c2)oc2cc(N(c4ccc5ccccc5c4)c4cccc5ccccc45)ccc23)c2ccccc2-c2ccccc2)cc1]

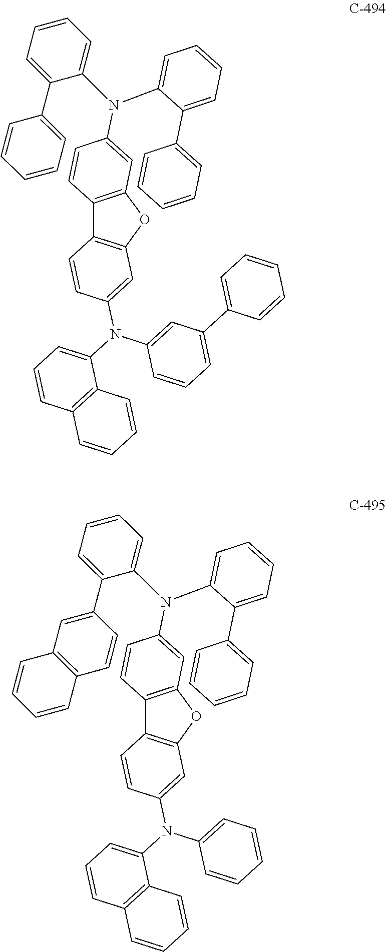 [SMILES: c1ccc(-c2cccc(N(c3ccc4c(c3)oc3cc(N(c5ccccc5-c5ccccc5)c5ccccc5-c5ccccc5)ccc34)c3cccc4ccccc34)c2)cc1 ; c1ccc(-c2ccccc2N(c2ccc3c(c2)oc2cc(N(c4ccccc4)c4cccc5ccccc45)ccc23)c2ccccc2-c2ccc3ccccc3c2)cc1]

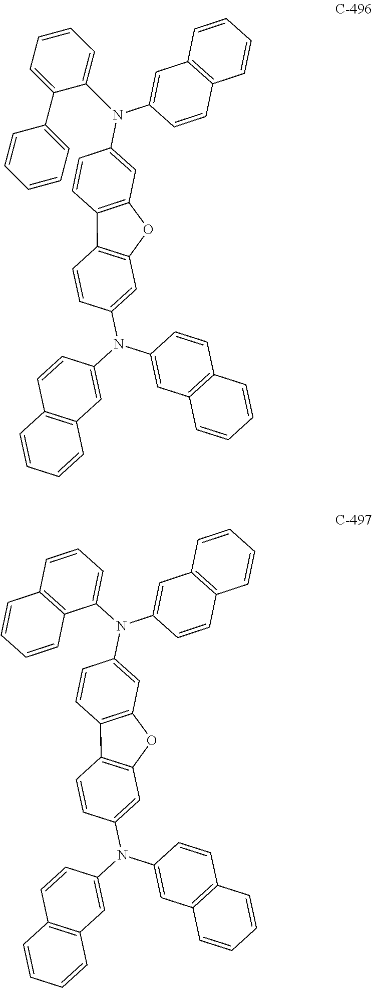 [SMILES: c1ccc(-c2ccccc2N(c2ccc3ccccc3c2)c2ccc3c(c2)oc2cc(N(c4ccc5ccccc5c4)c4ccc5ccccc5c4)ccc23)cc1 ; c1ccc2cc(N(c3ccc4ccccc4c3)c3ccc4c(c3)oc3cc(N(c5ccc6ccccc6c5)c5cccc6ccccc56)ccc34)ccc2c1]

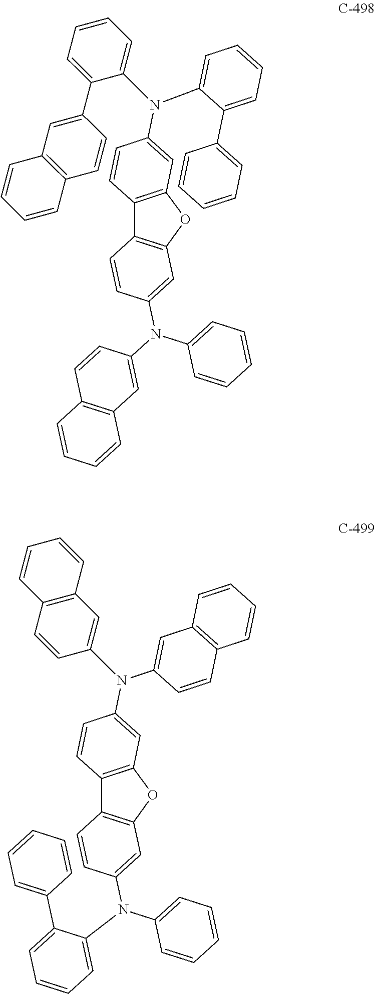 [SMILES: c1ccc(-c2ccccc2N(c2ccc3c(c2)oc2cc(N(c4ccccc4)c4ccc5ccccc5c4)ccc23)c2ccccc2-c2ccc3ccccc3c2)cc1 ; c1ccc(-c2ccccc2N(c2ccccc2)c2ccc3c(c2)oc2cc(N(c4ccc5ccccc5c4)c4ccc5ccccc5c4)ccc23)cc1]

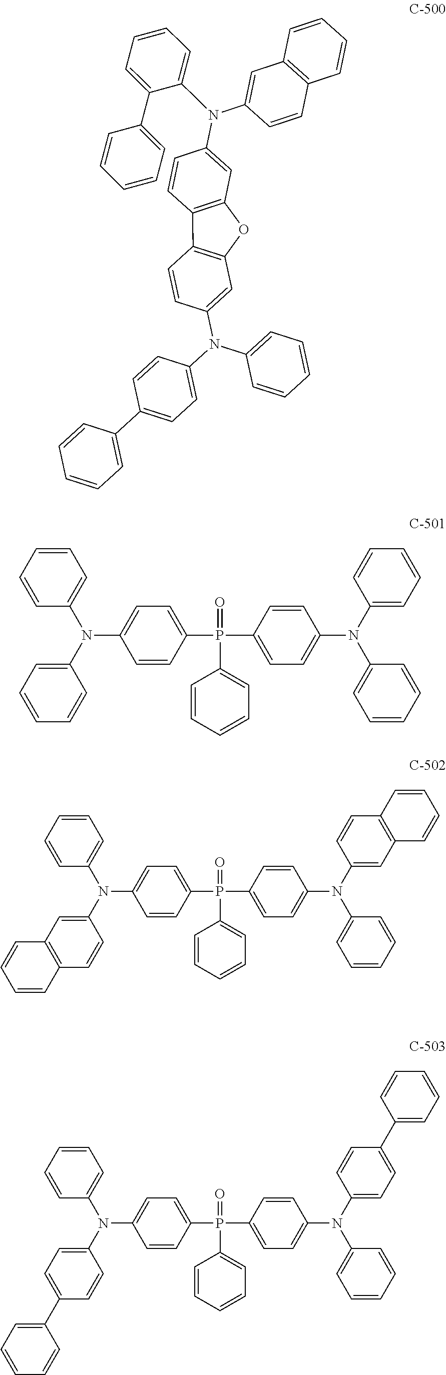 [SMILES: c1ccc(-c2ccc(N(c3ccccc3)c3ccc4c(c3)oc3cc(N(c5ccc6ccccc6c5)c5ccccc5-c5ccccc5)ccc34)cc2)cc1 ; O=P(c1ccccc1)(c1ccc(N(c2ccccc2)c2ccccc2)cc1)c1ccc(N(c2ccccc2)c2ccccc2)cc1 ; O=P(c1ccccc1)(c1ccc(N(c2ccccc2)c2ccc3ccccc3c2)cc1)c1ccc(N(c2ccccc2)c2ccc3ccccc3c2)cc1 ; O=P(c1ccccc1)(c1ccc(N(c2ccccc2)c2ccc(-c3ccccc3)cc2)cc1)c1ccc(N(c2ccccc2)c2ccc(-c3ccccc3)cc2)cc1]

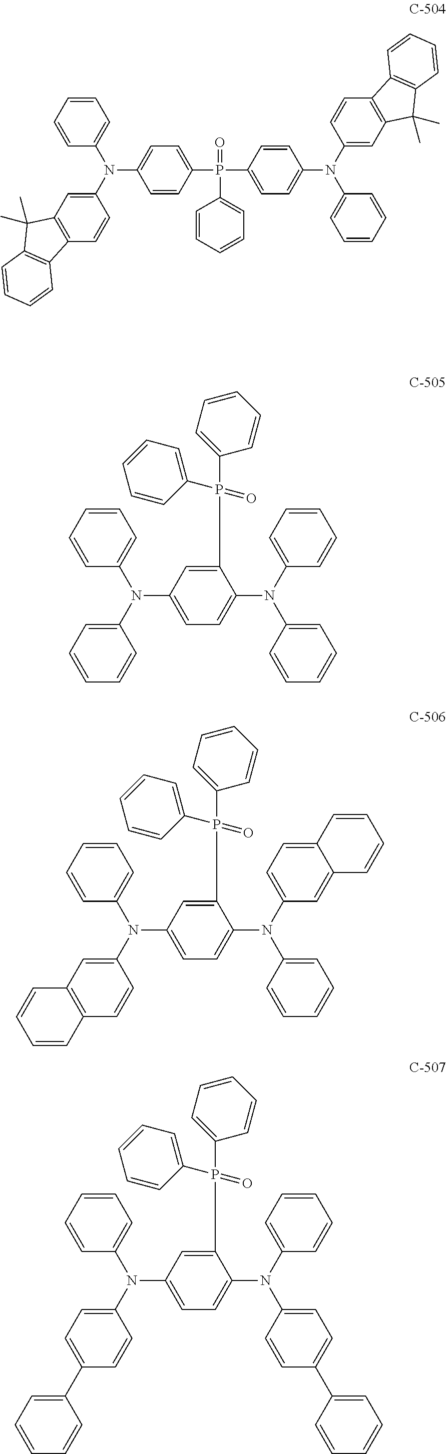

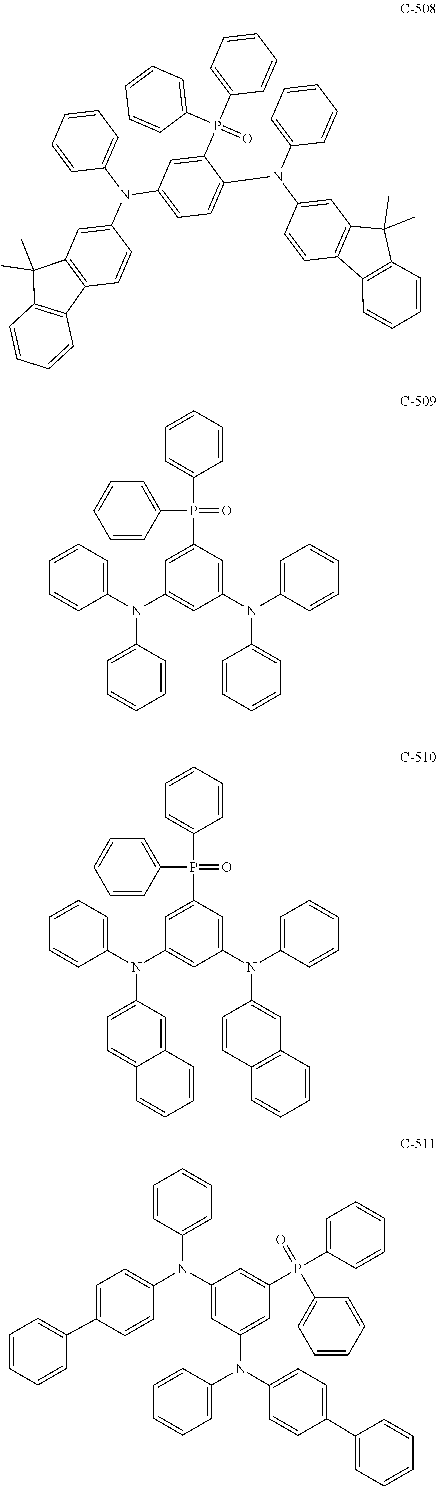 [SMILES: CC1(C)c2ccccc2-c2ccc(N(c3ccccc3)c3ccc(N(c4ccccc4)c4ccc5c(c4)C(C)(C)c4ccccc4-5)c(P(=O)(c4ccccc4)c4ccccc4)c3)cc21 ; O=P(c1ccccc1)(c1ccccc1)c1cc(N(c2ccccc2)c2ccccc2)cc(N(c2ccccc2)c2ccccc2)c1 ; O=P(c1ccccc1)(c1ccccc1)c1cc(N(c2ccccc2)c2ccc3ccccc3c2)cc(N(c2ccccc2)c2ccc3ccccc3c2)c1 ; O=P(c1ccccc1)(c1ccccc1)c1cc(N(c2ccccc2)c2ccc(-c3ccccc3)cc2)cc(N(c2ccccc2)c2ccc(-c3ccccc3)cc2)c1]

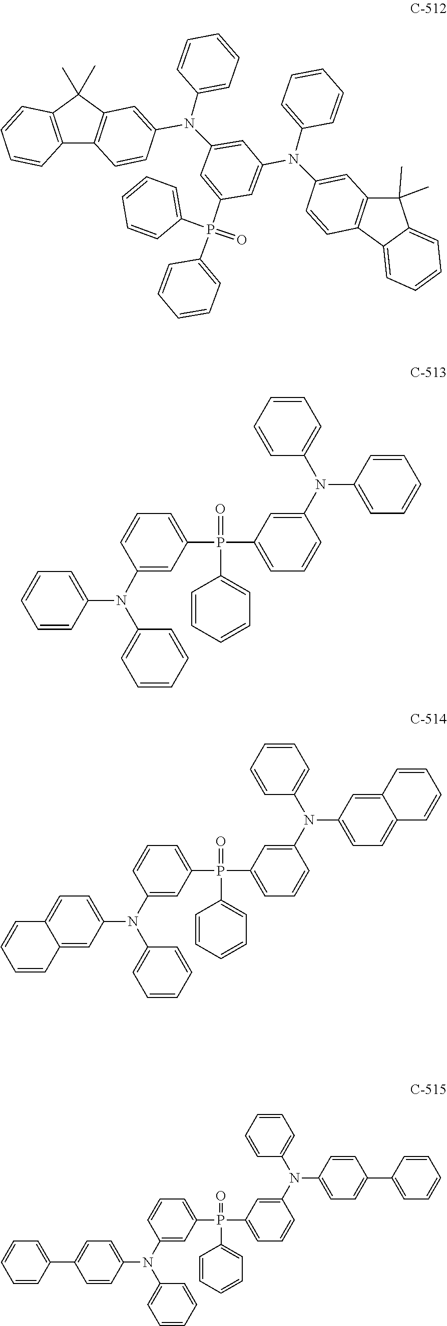 [SMILES: CC1(C)c2ccccc2-c2ccc(N(c3ccccc3)c3cc(N(c4ccccc4)c4ccc5c(c4)C(C)(C)c4ccccc4-5)cc(P(=O)(c4ccccc4)c4ccccc4)c3)cc21 ; O=P(c1ccccc1)(c1cccc(N(c2ccccc2)c2ccccc2)c1)c1cccc(N(c2ccccc2)c2ccccc2)c1 ; O=P(c1ccccc1)(c1cccc(N(c2ccccc2)c2ccc3ccccc3c2)c1)c1cccc(N(c2ccccc2)c2ccc3ccccc3c2)c1 ; O=P(c1ccccc1)(c1cccc(N(c2ccccc2)c2ccc(-c3ccccc3)cc2)c1)c1cccc(N(c2ccccc2)c2ccc(-c3ccccc3)cc2)c1]

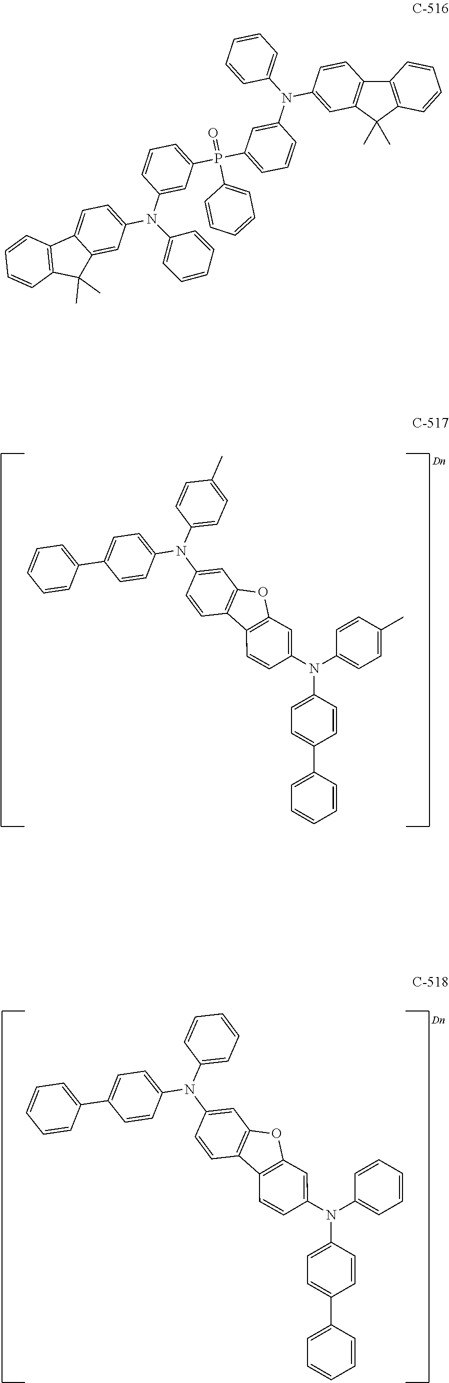

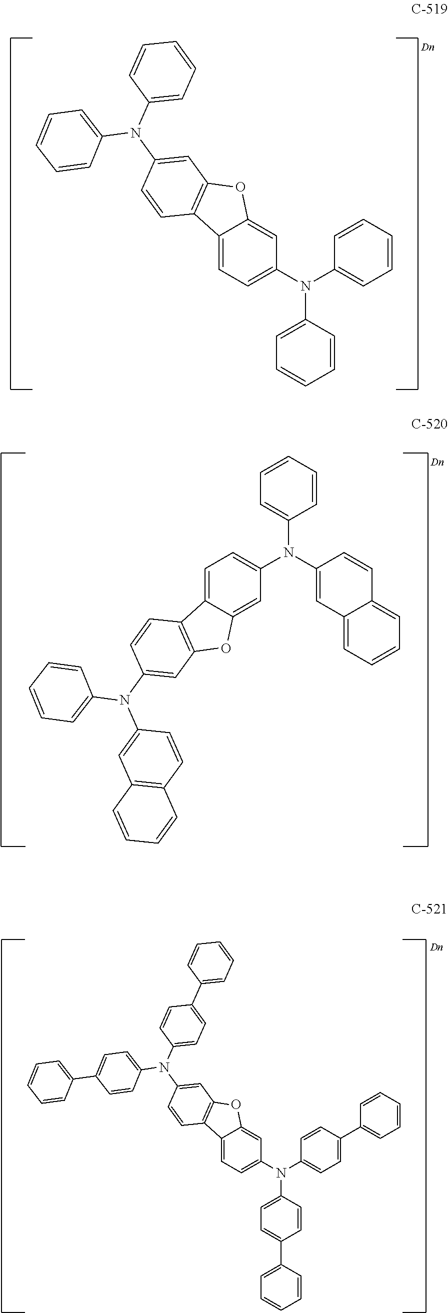

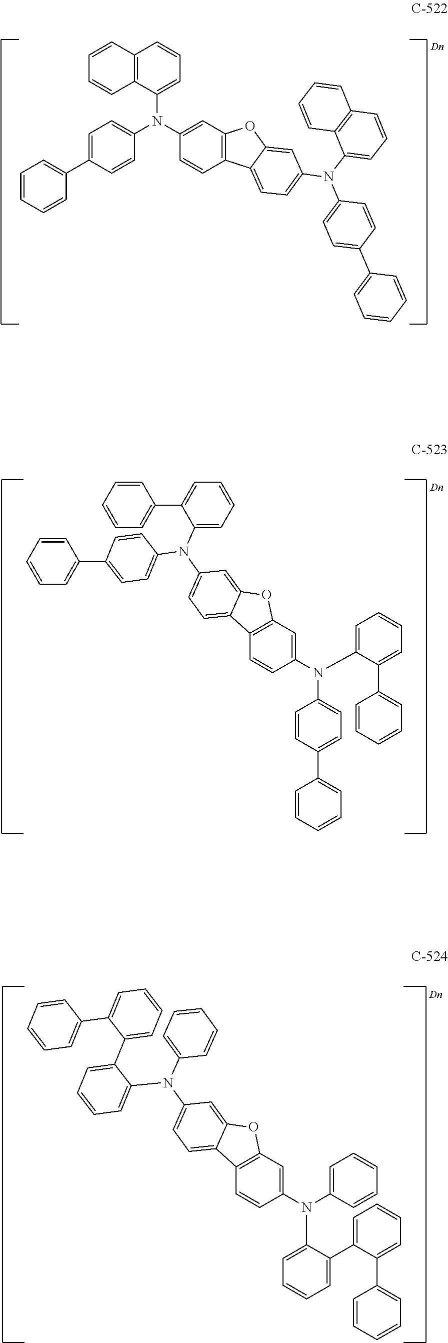

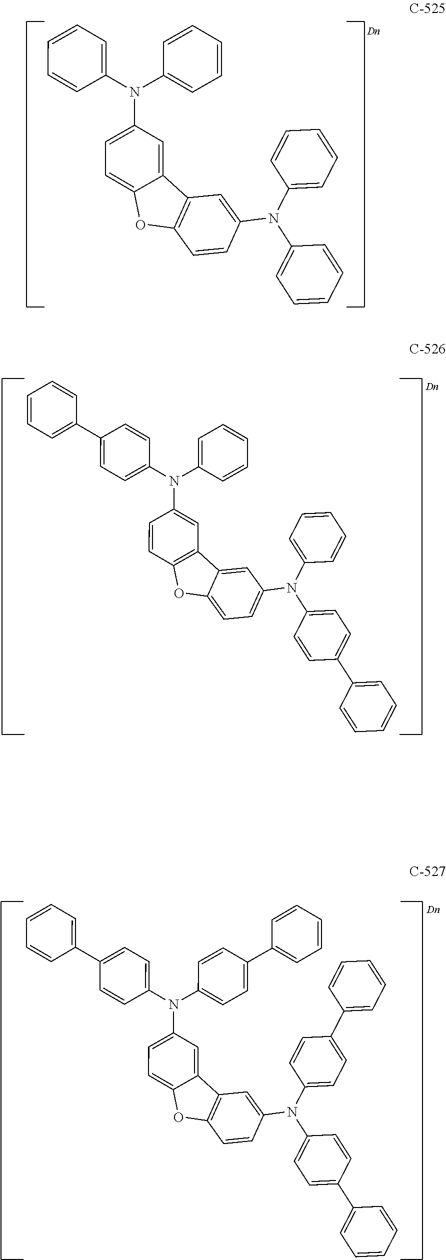

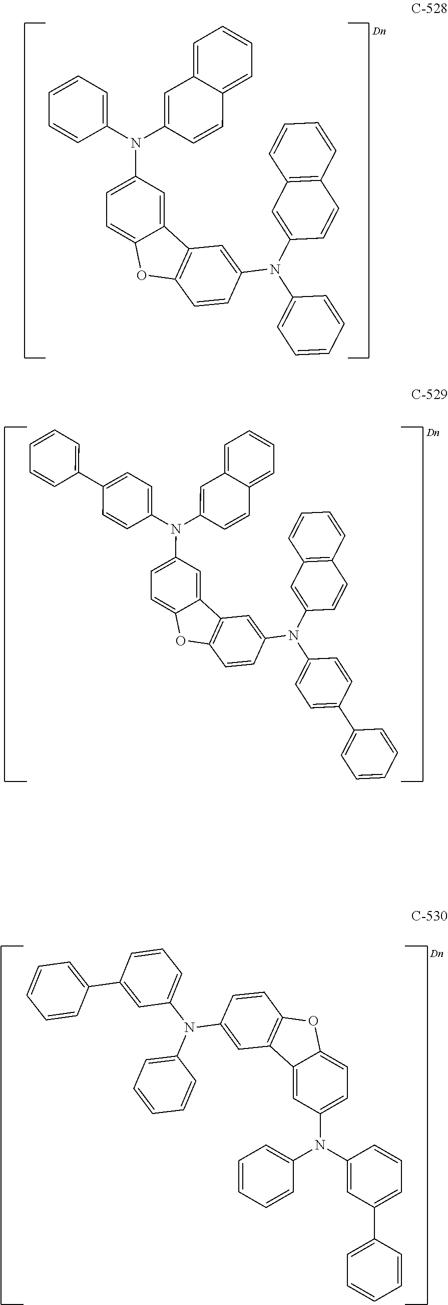

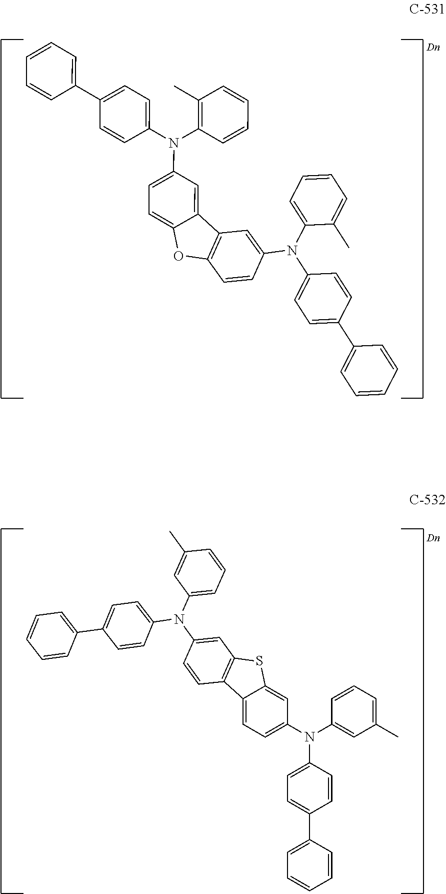

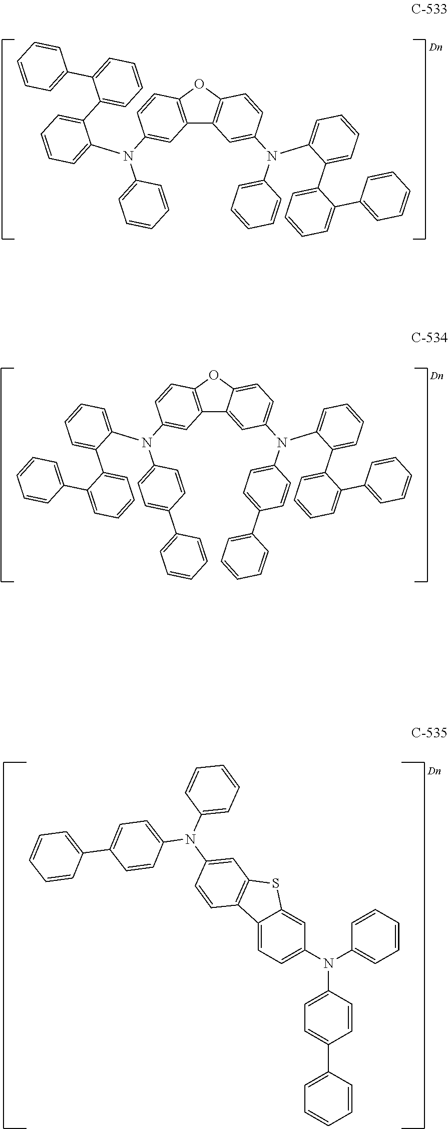

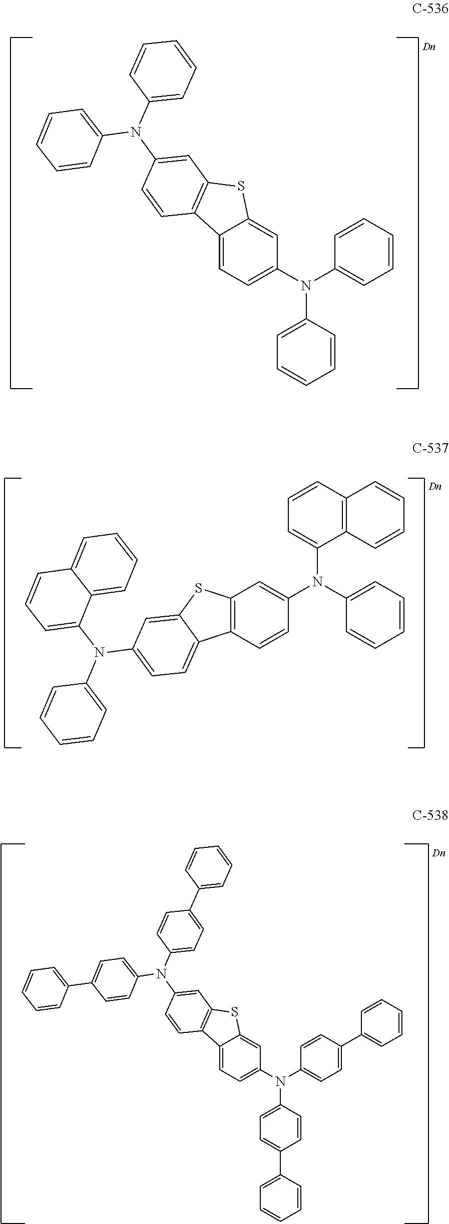

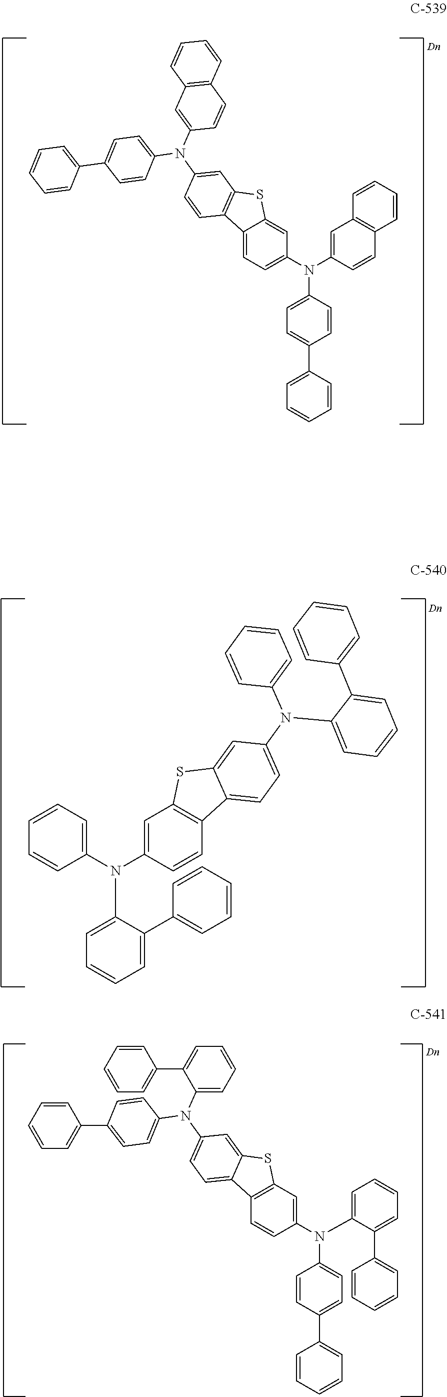

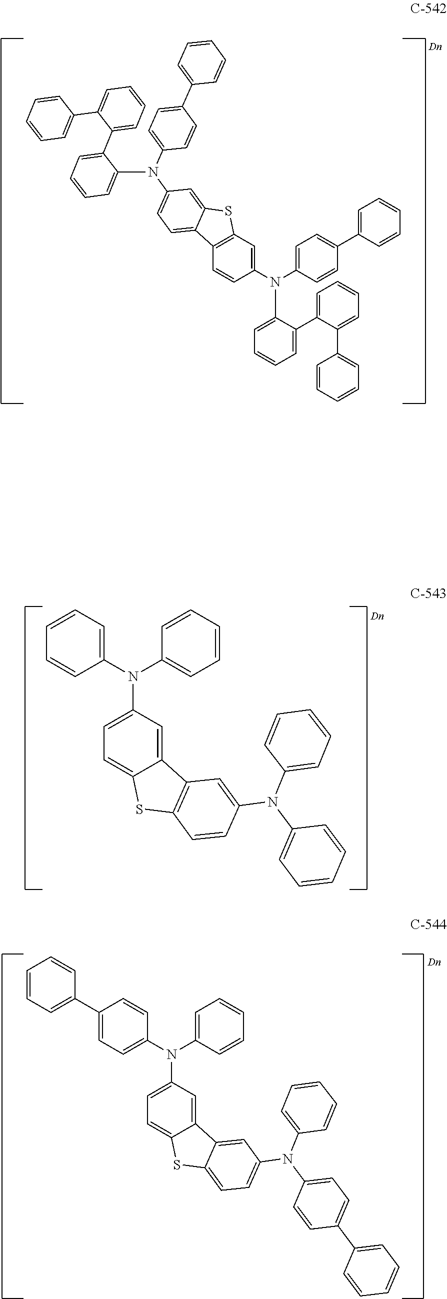

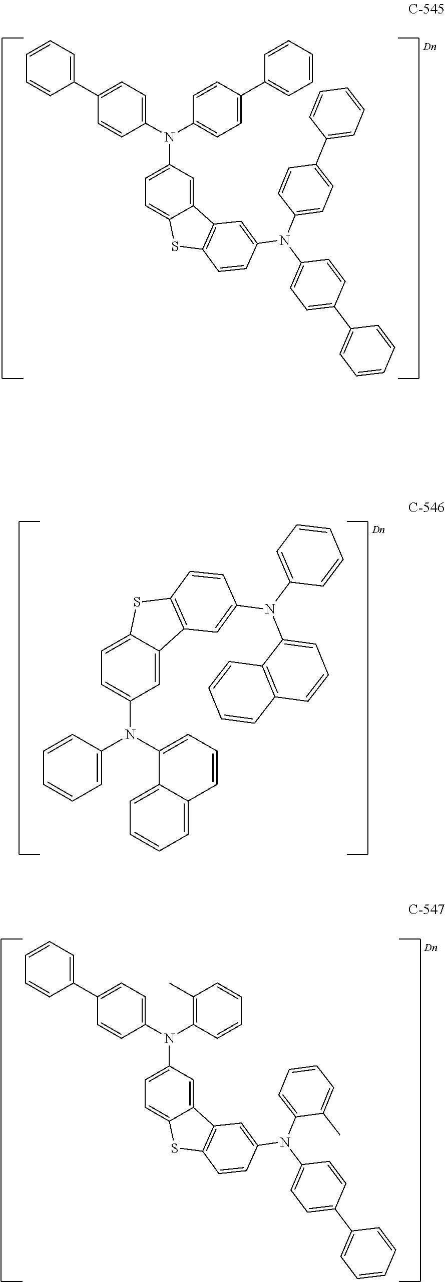

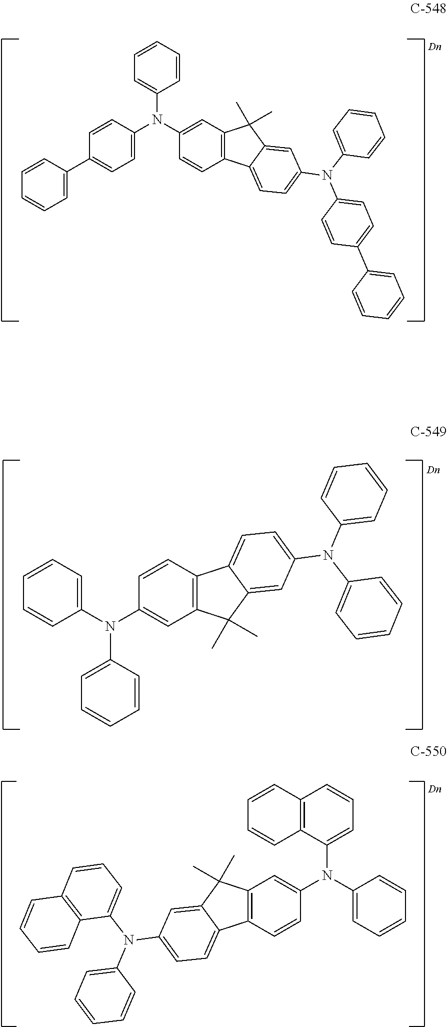

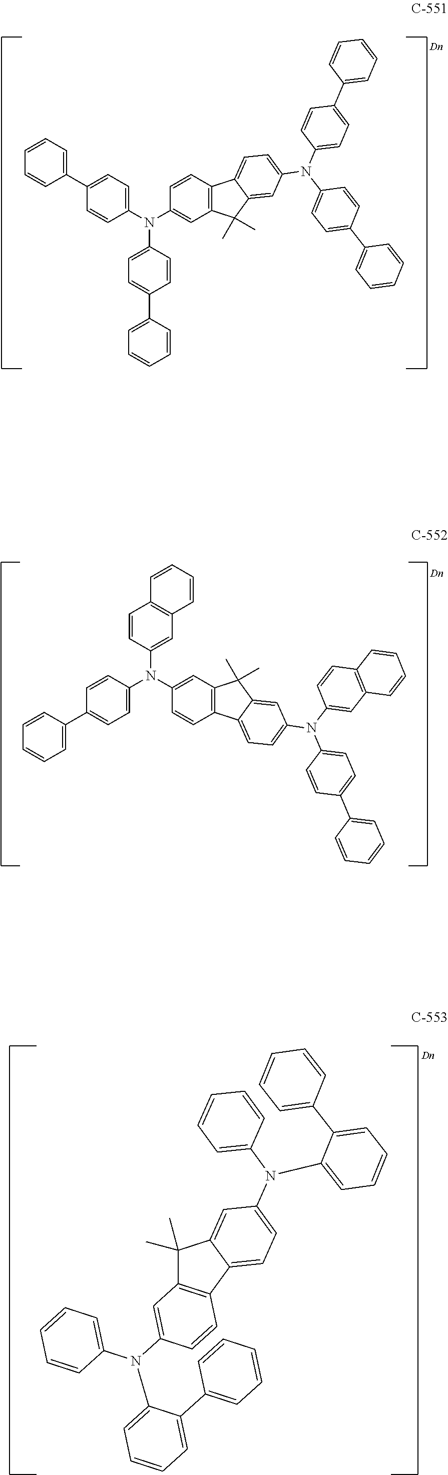

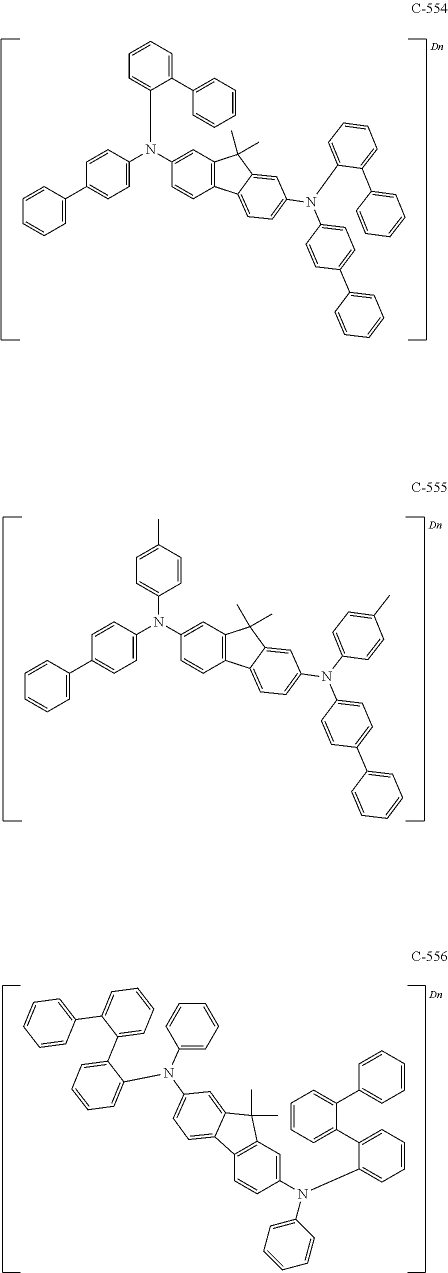

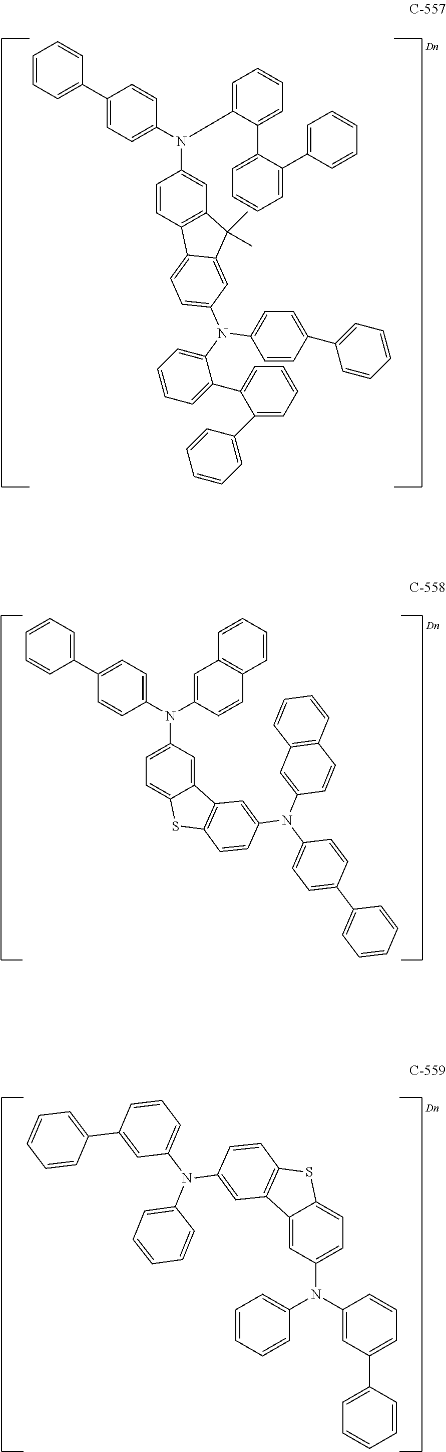

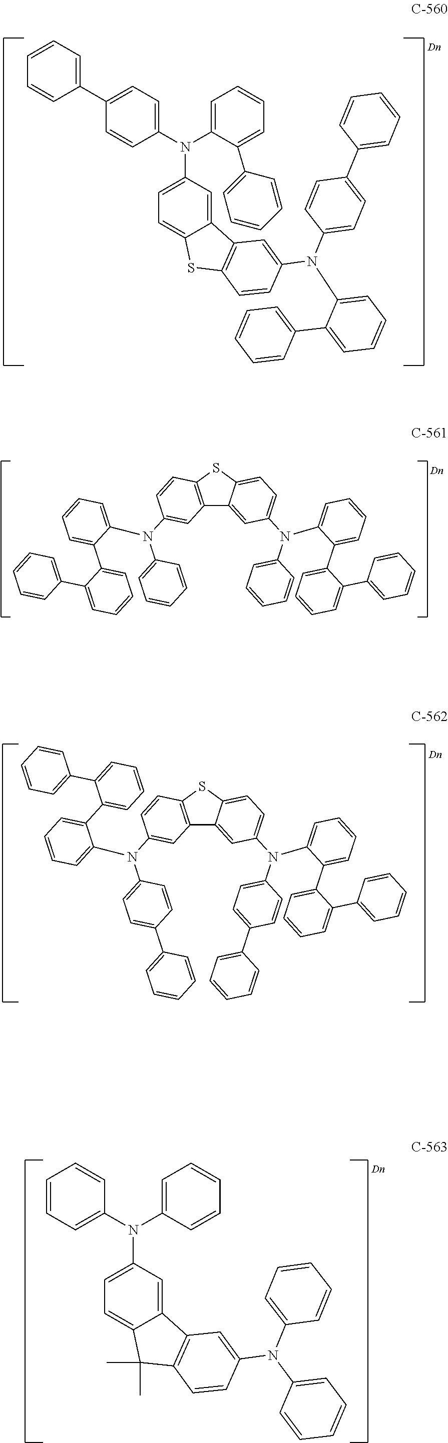

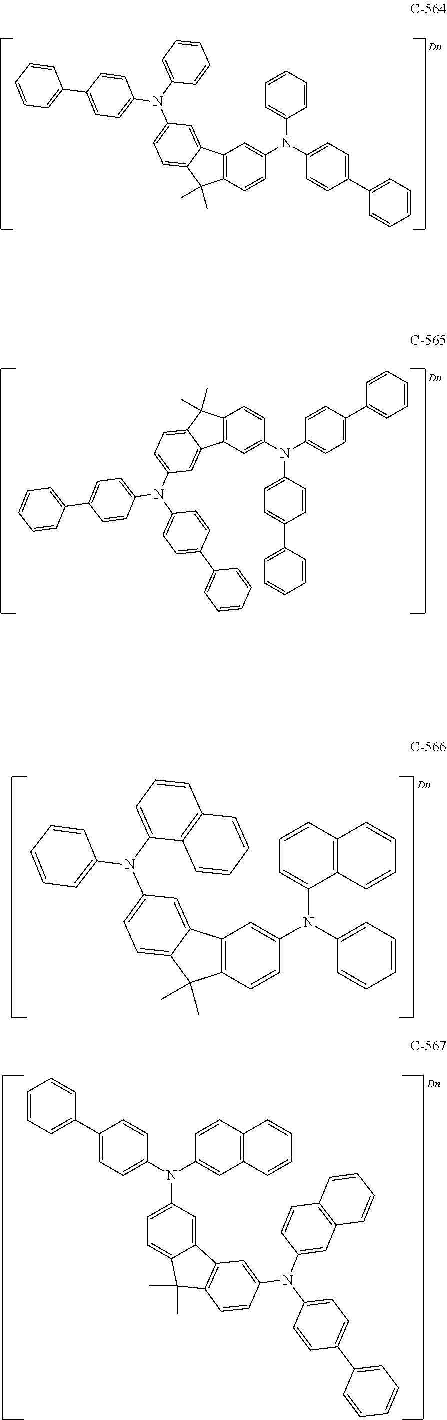

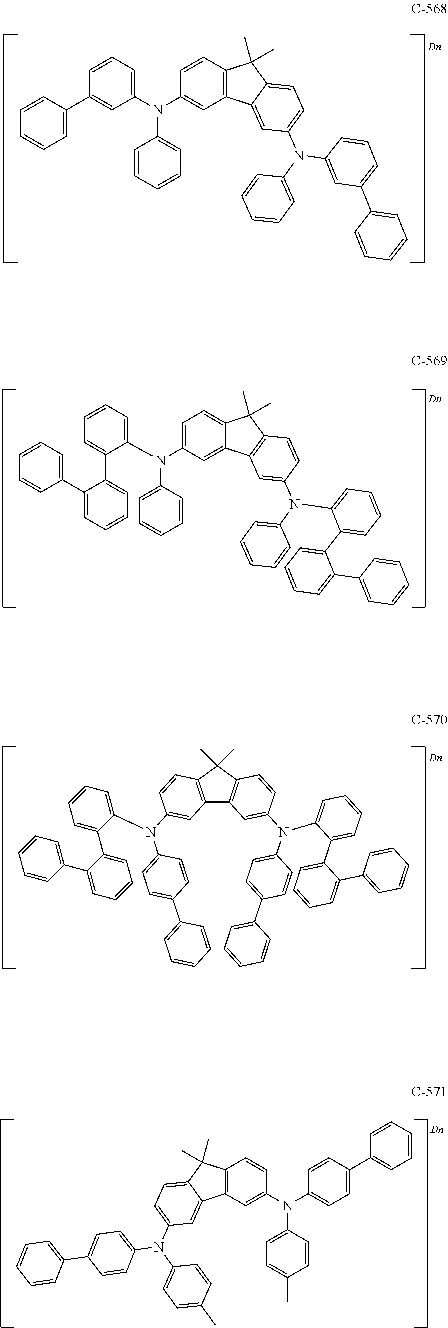

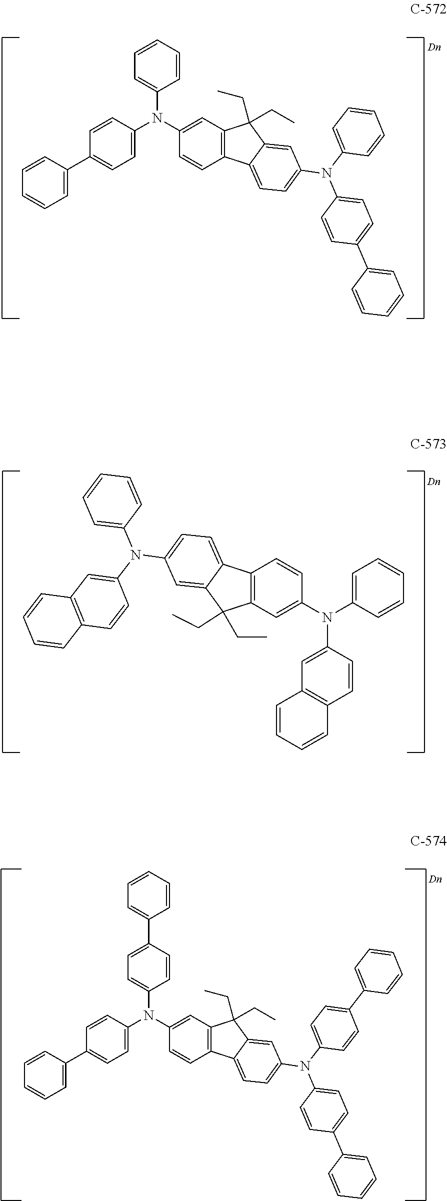

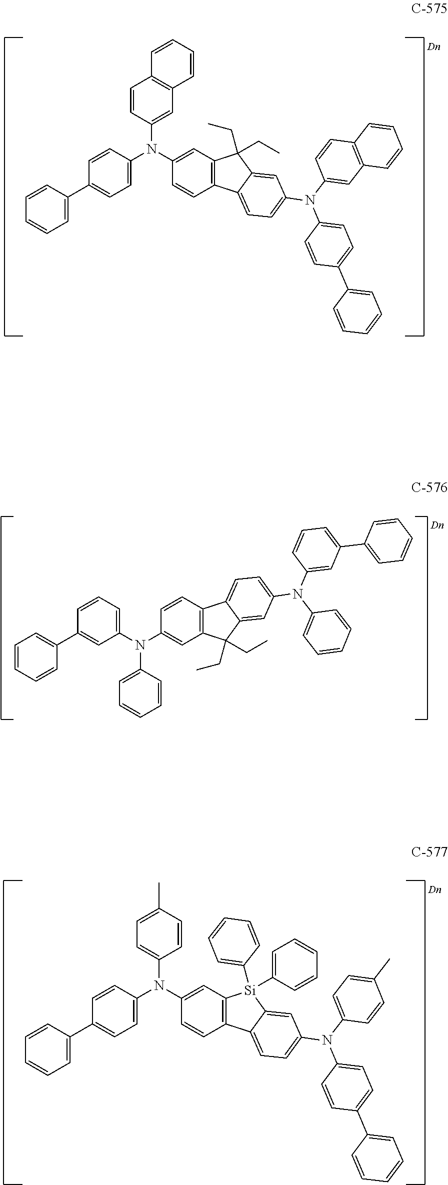

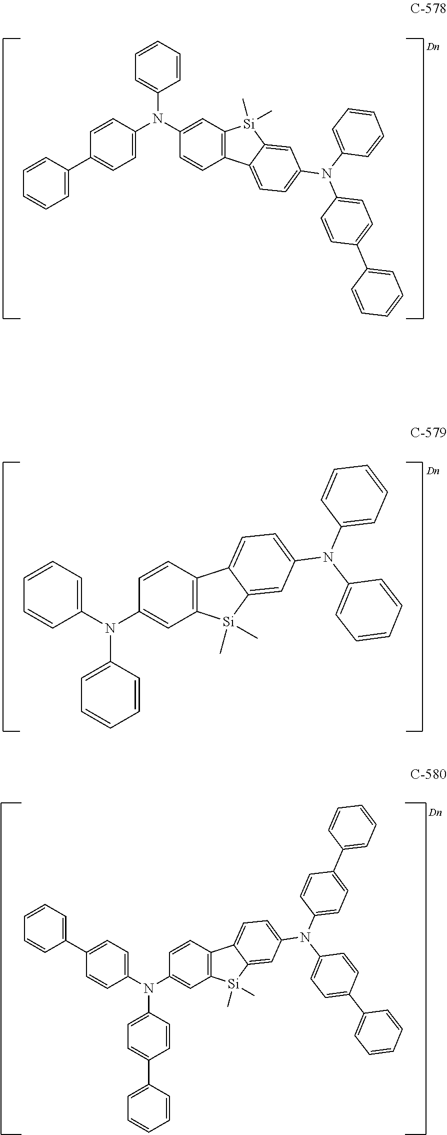

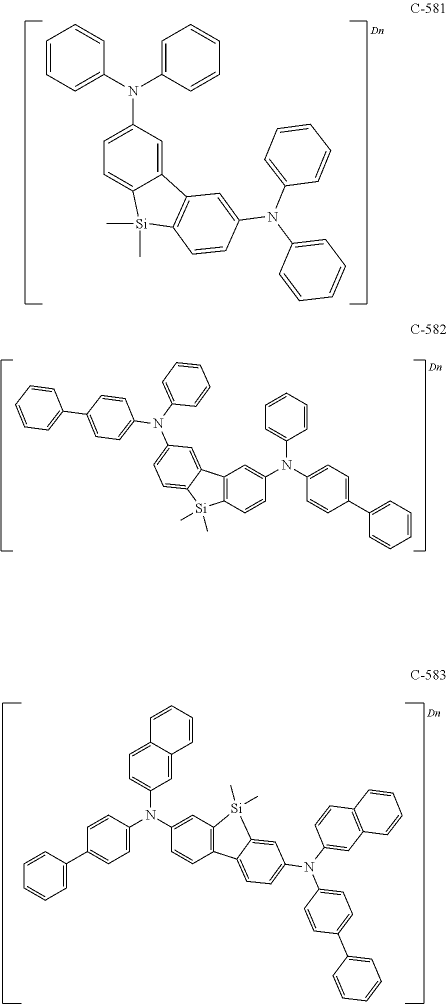

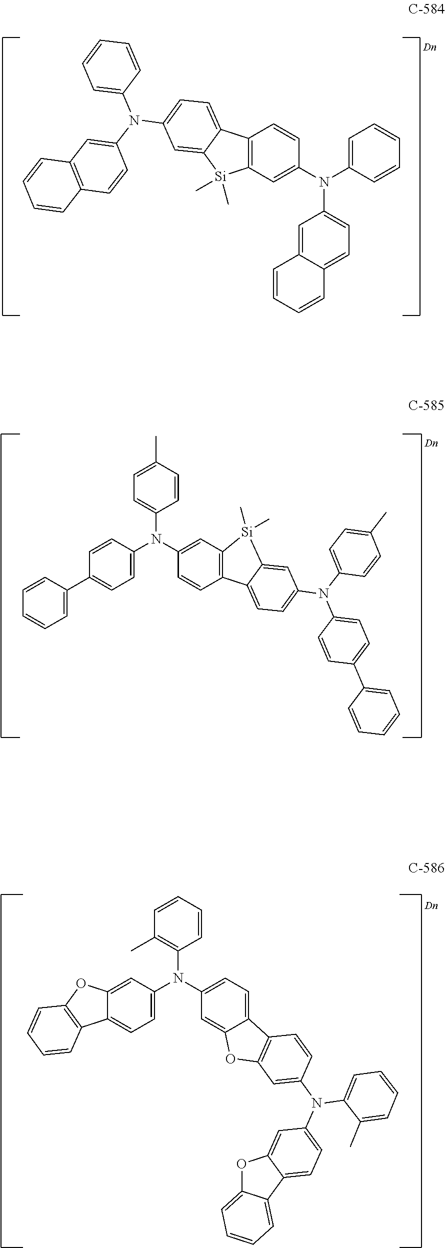

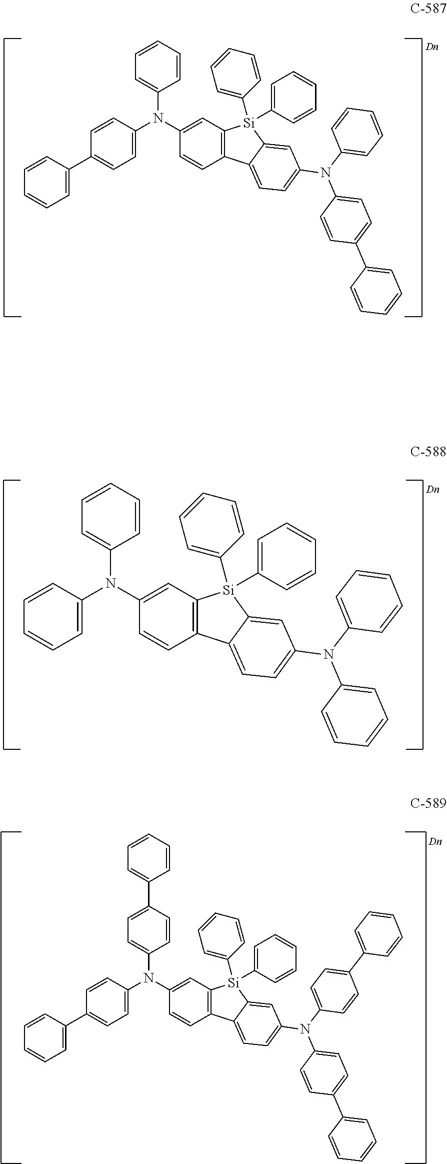

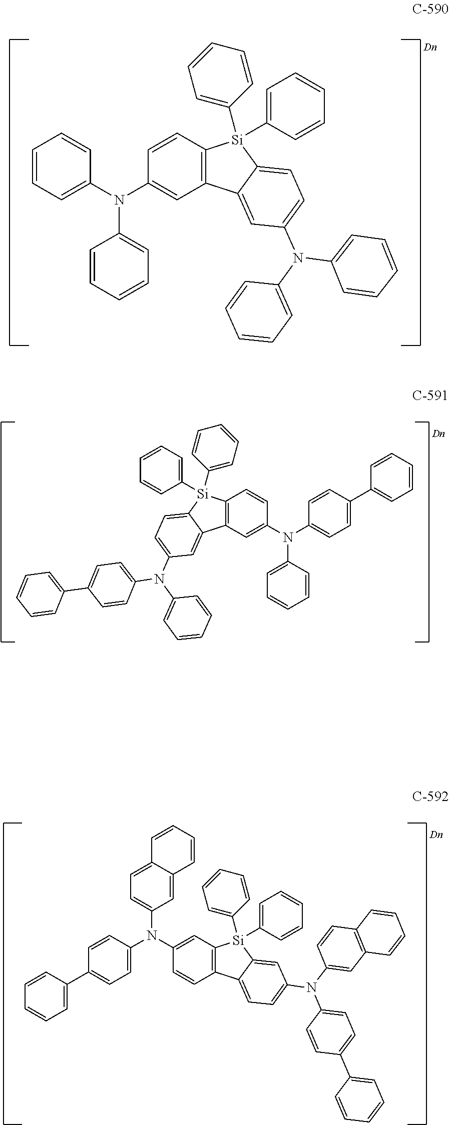

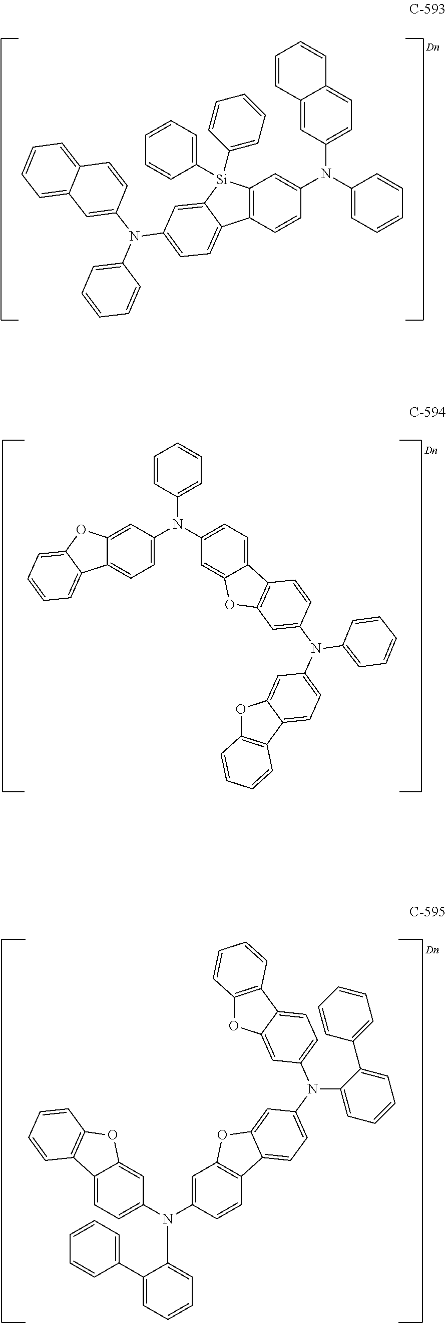

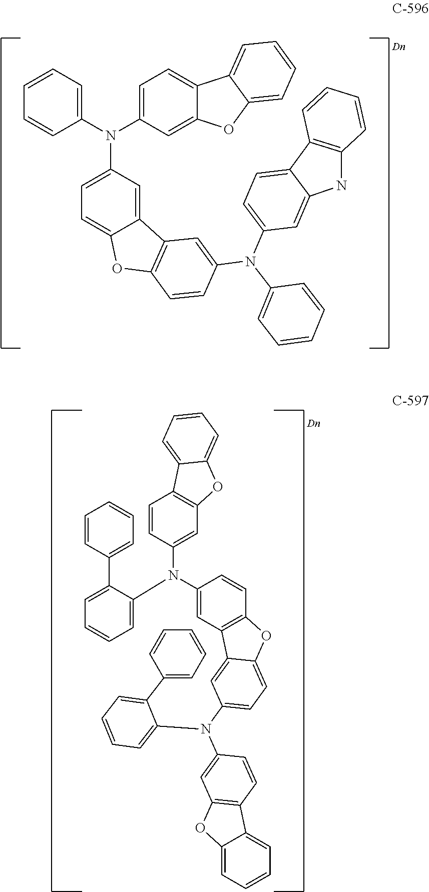

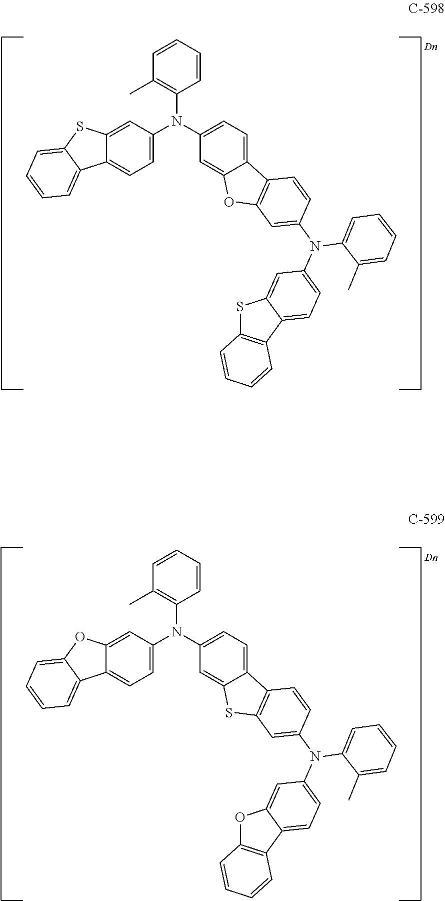

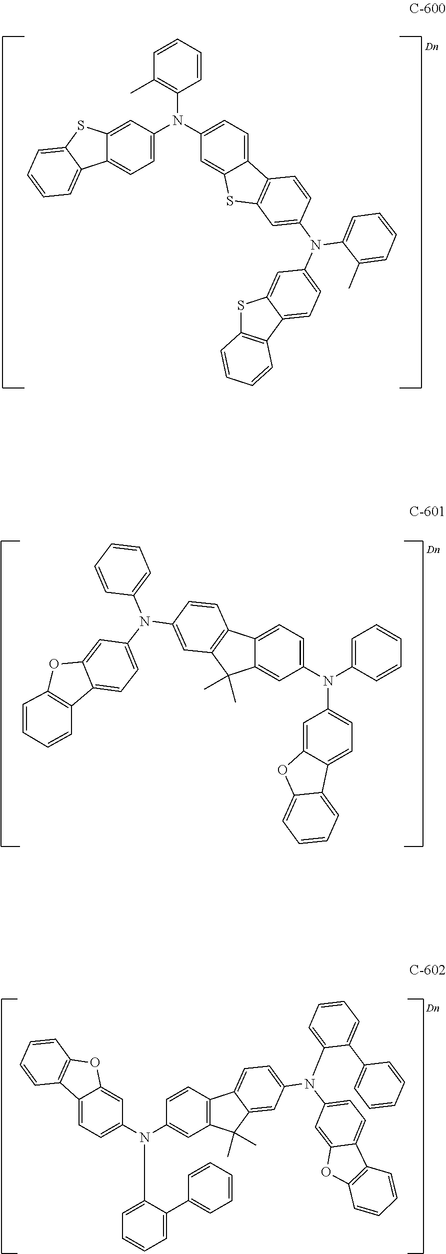

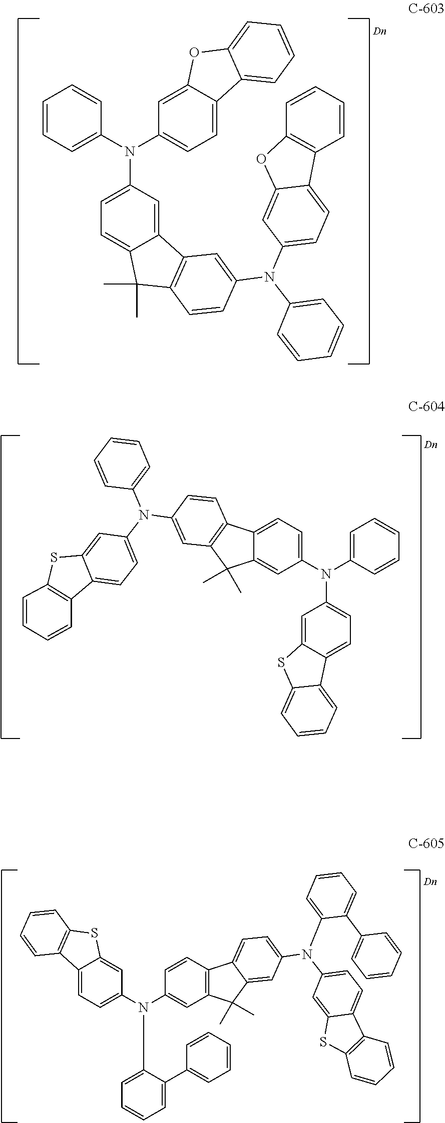

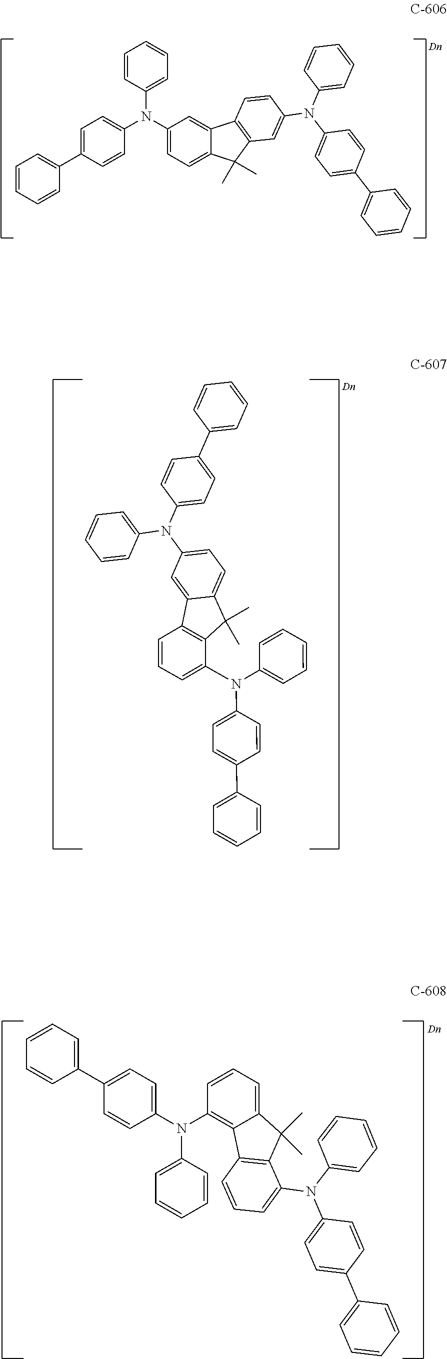

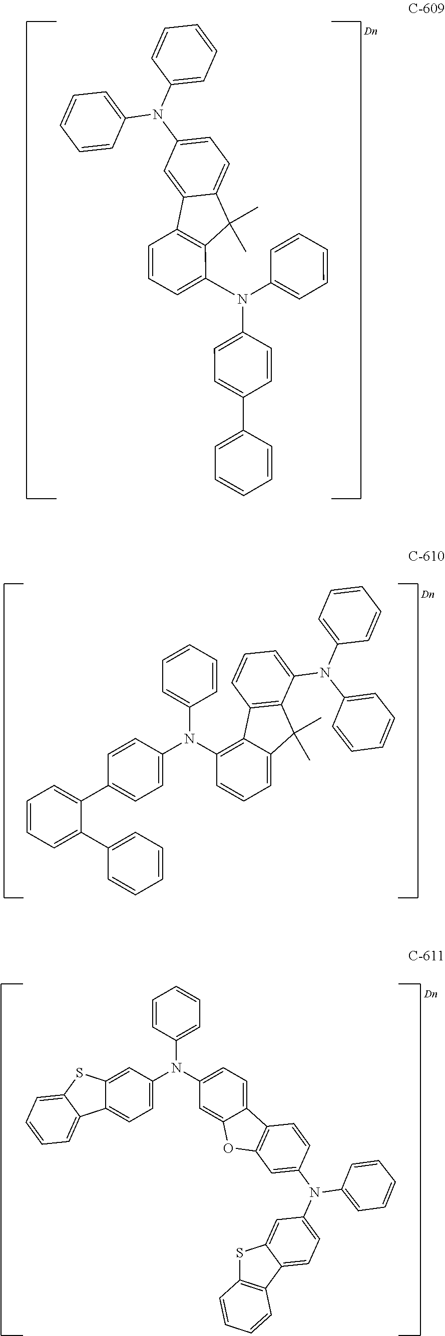

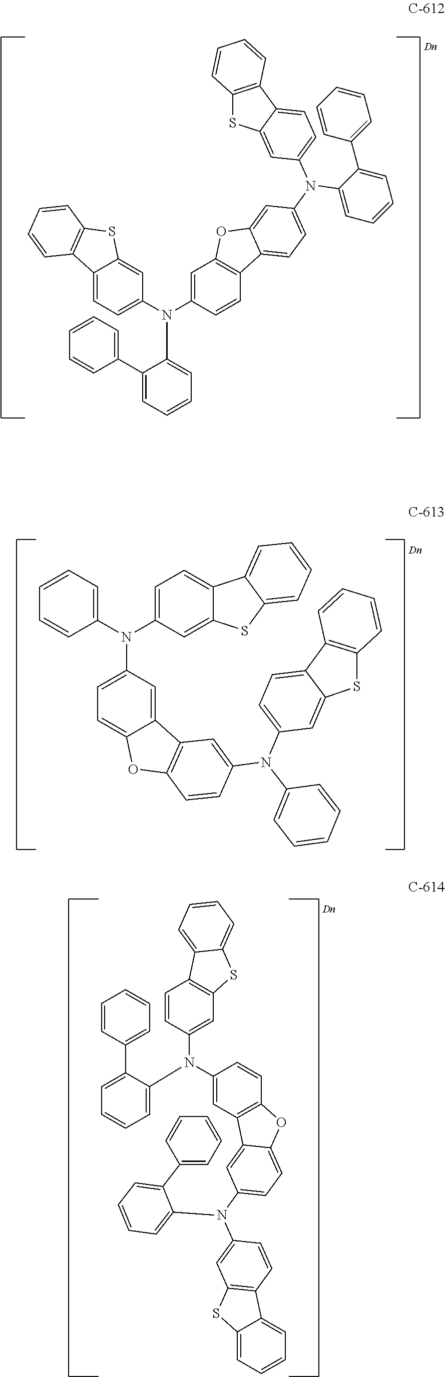

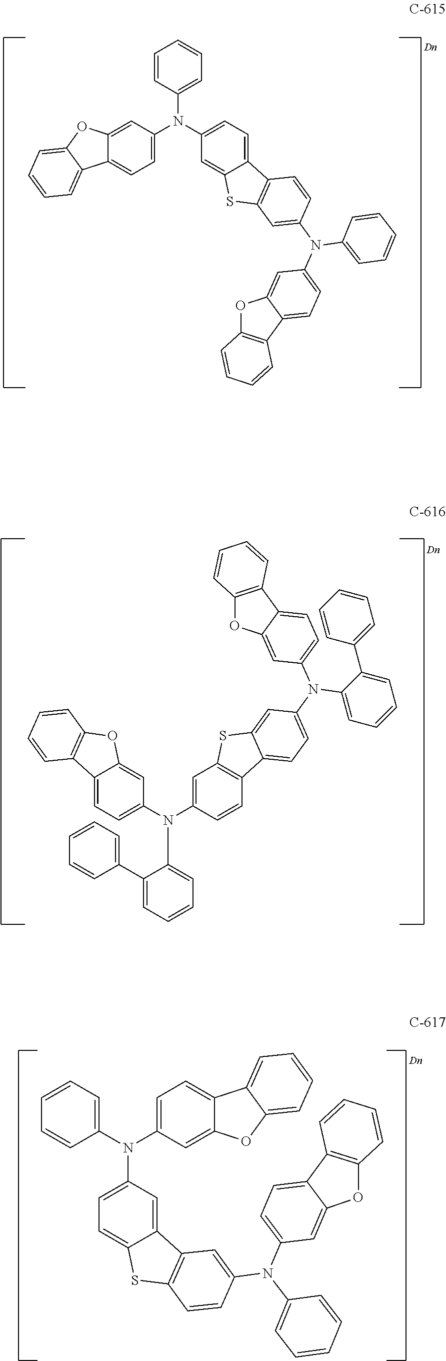

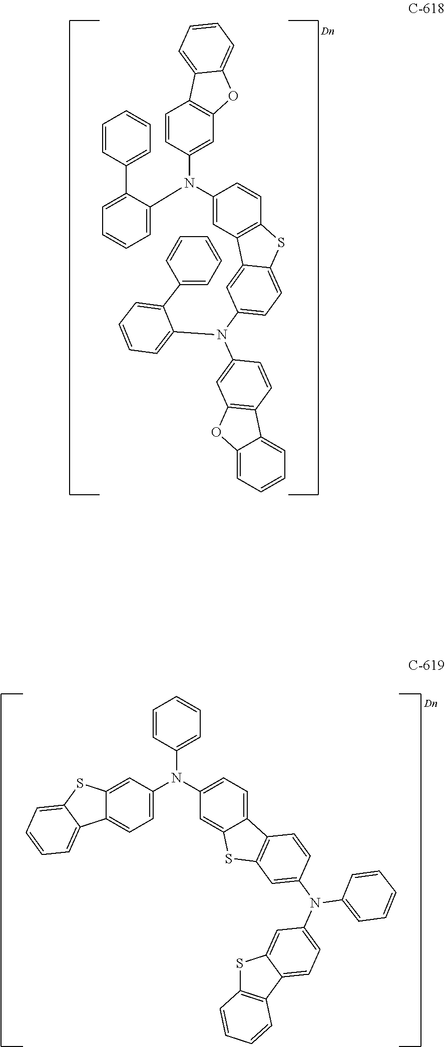

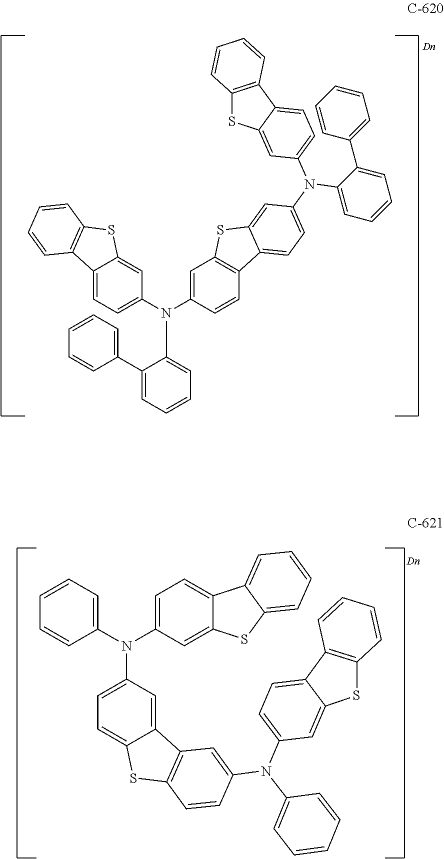

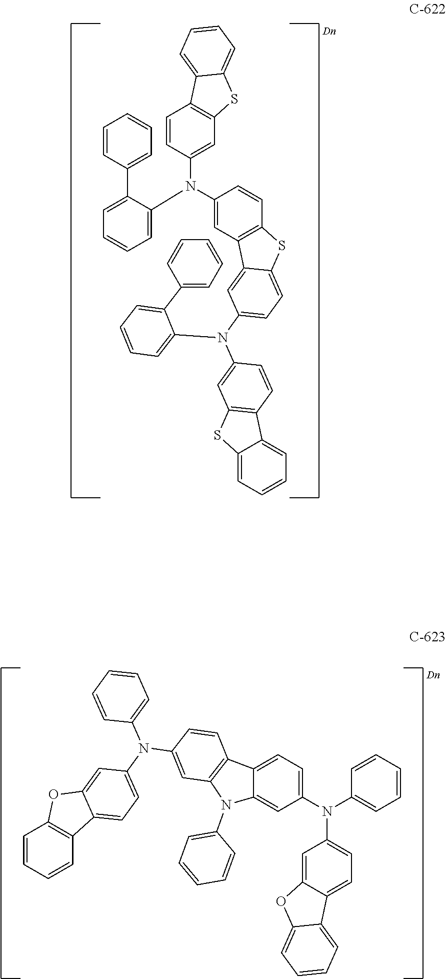

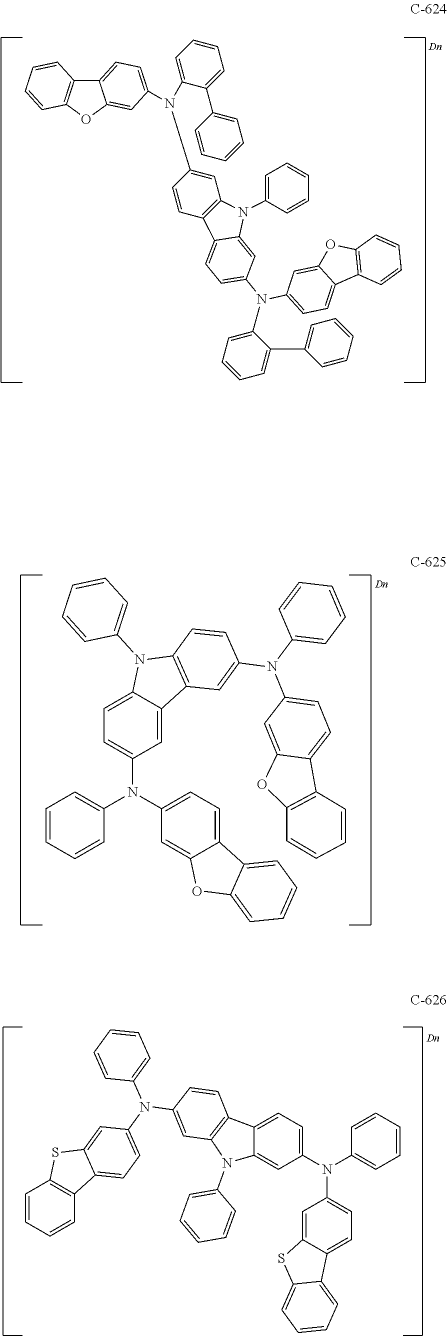

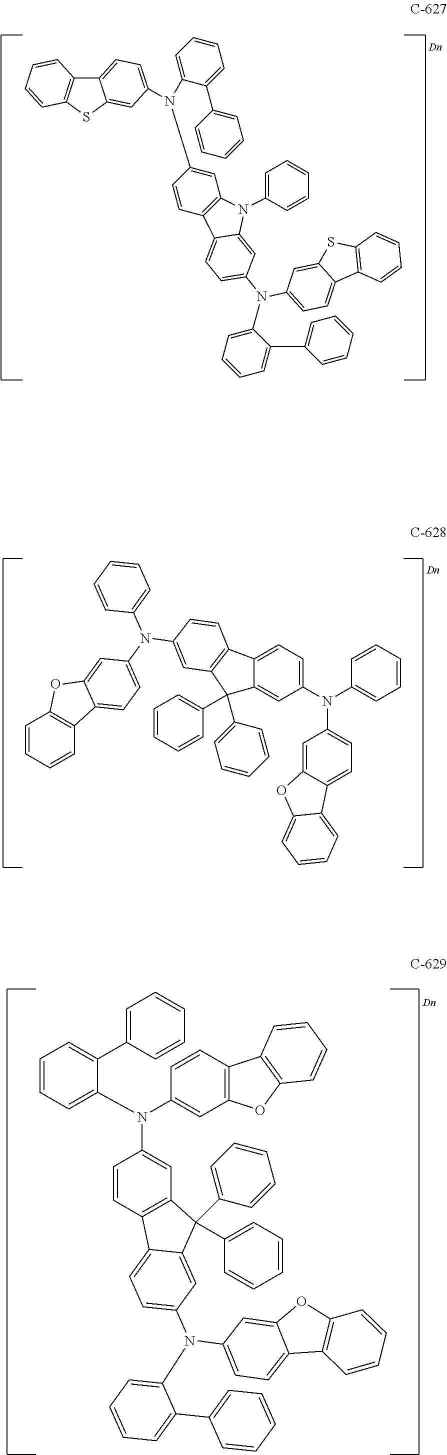

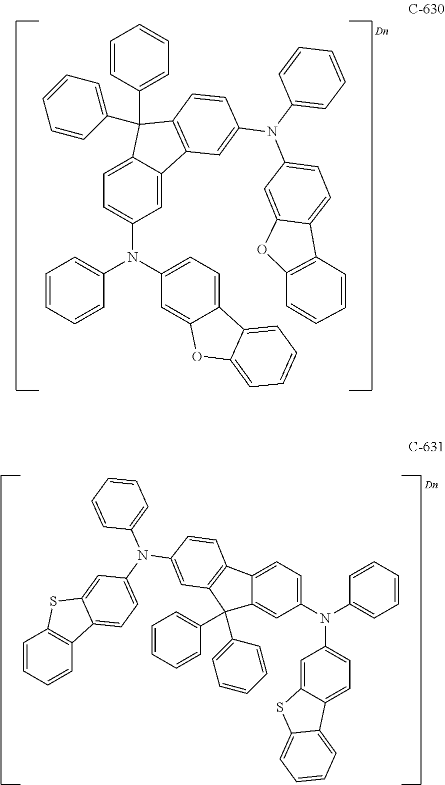

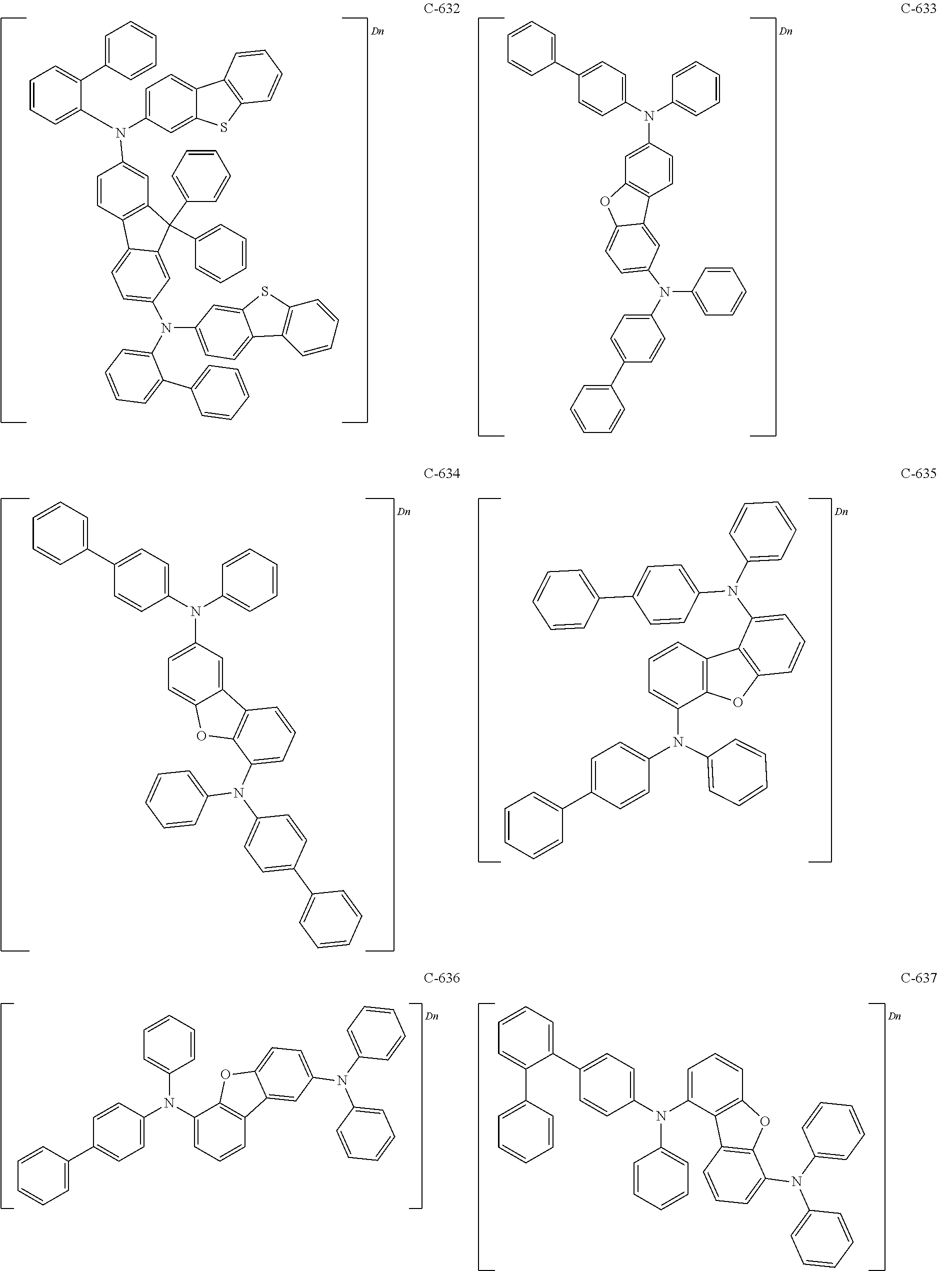

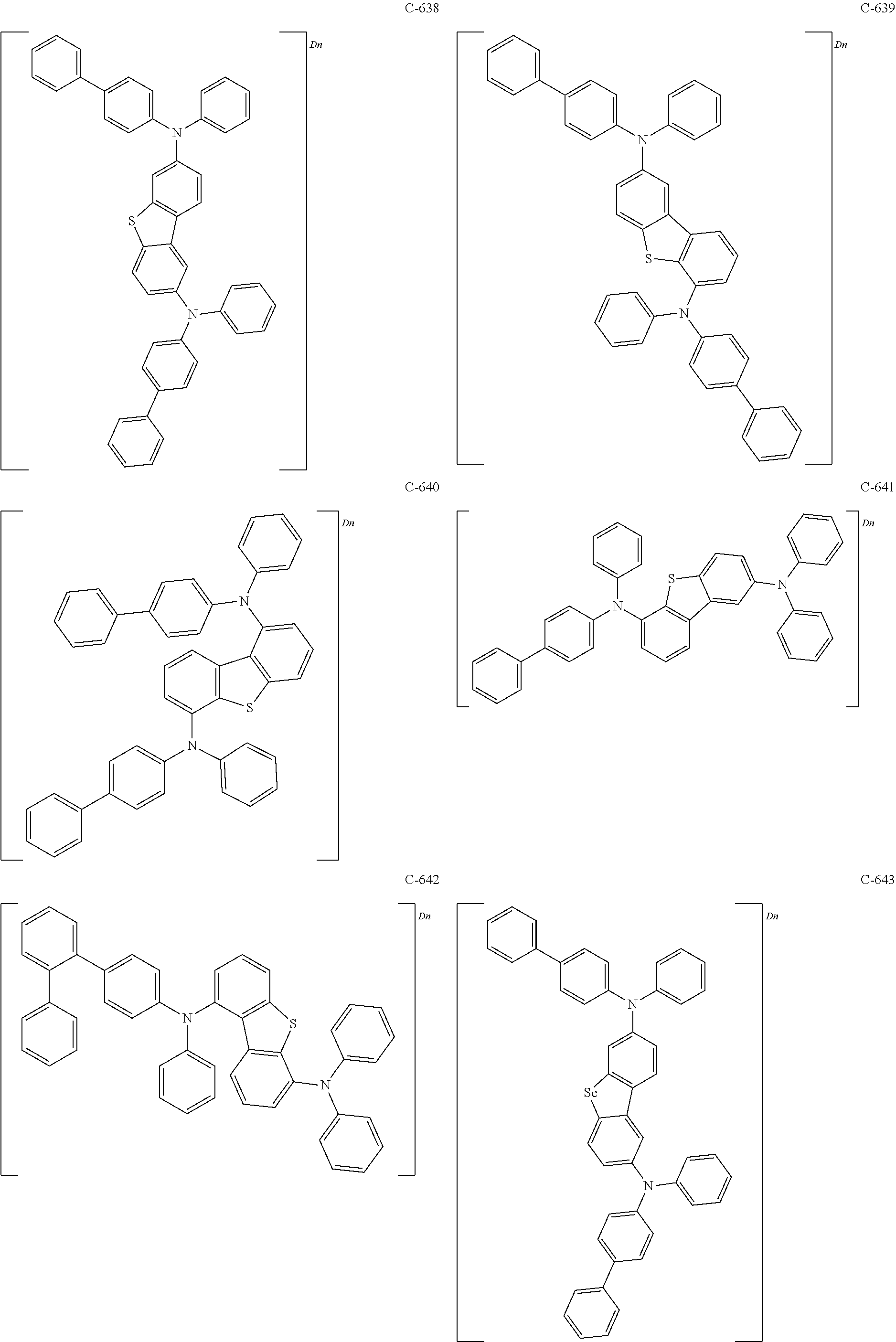

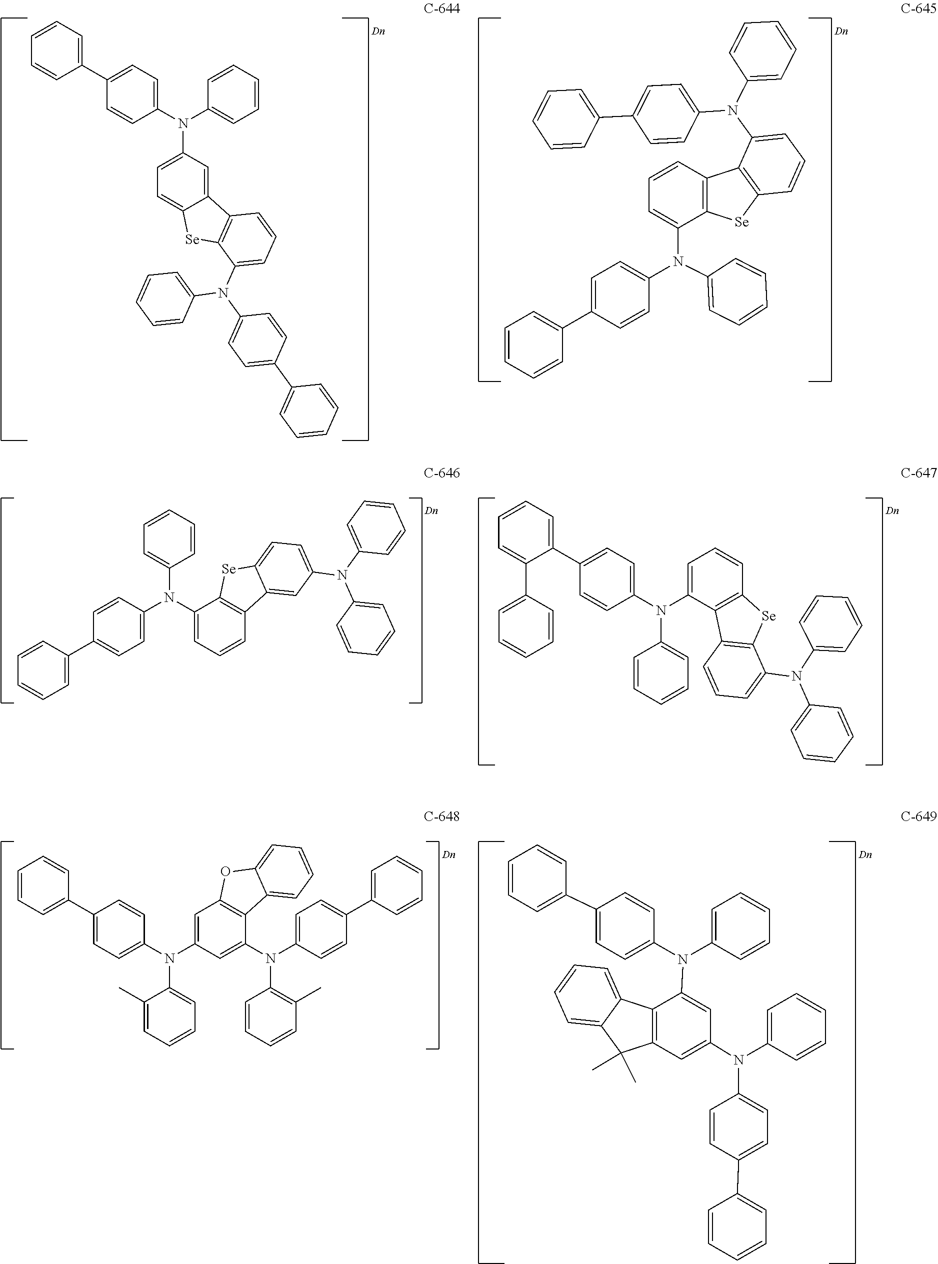

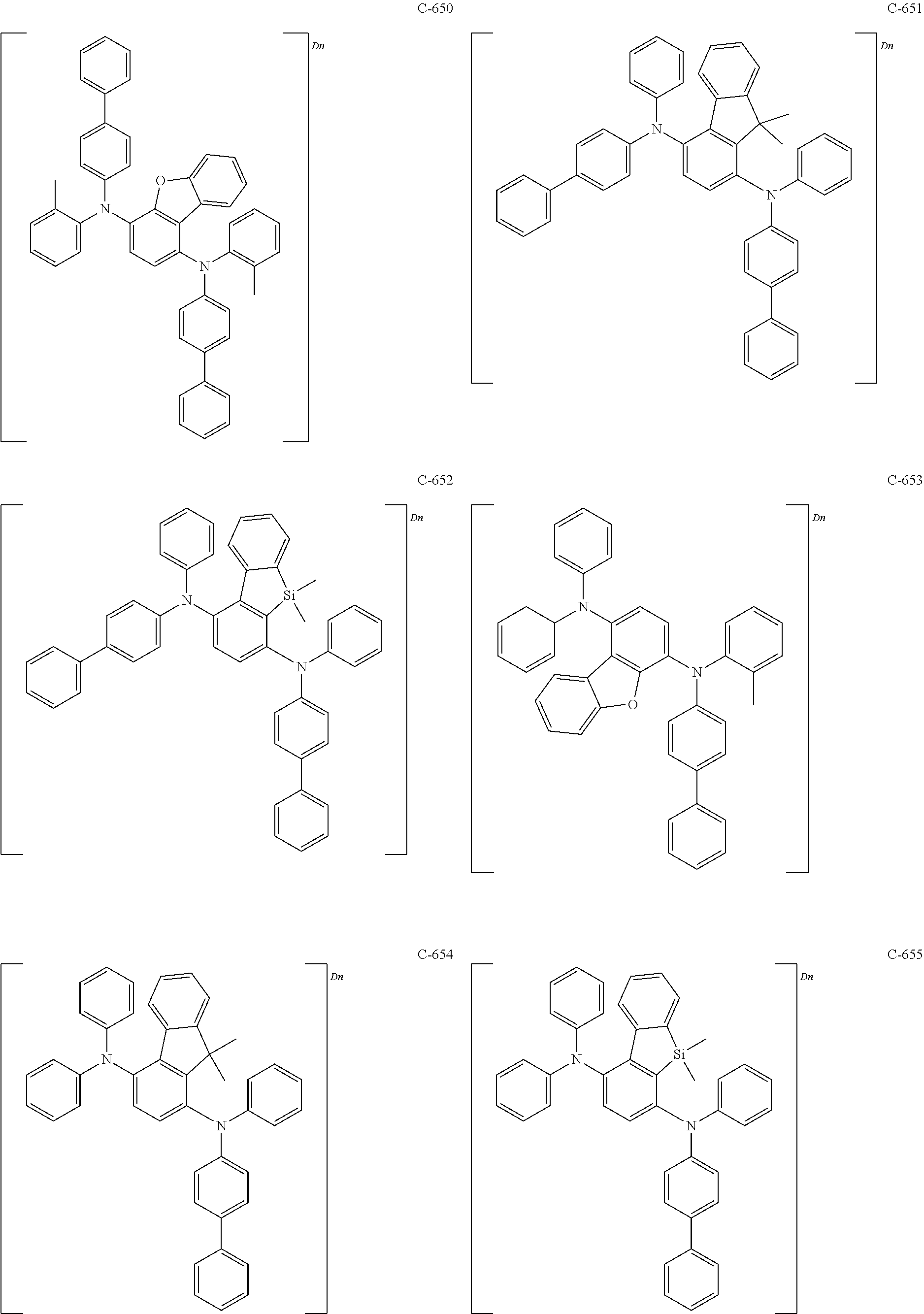

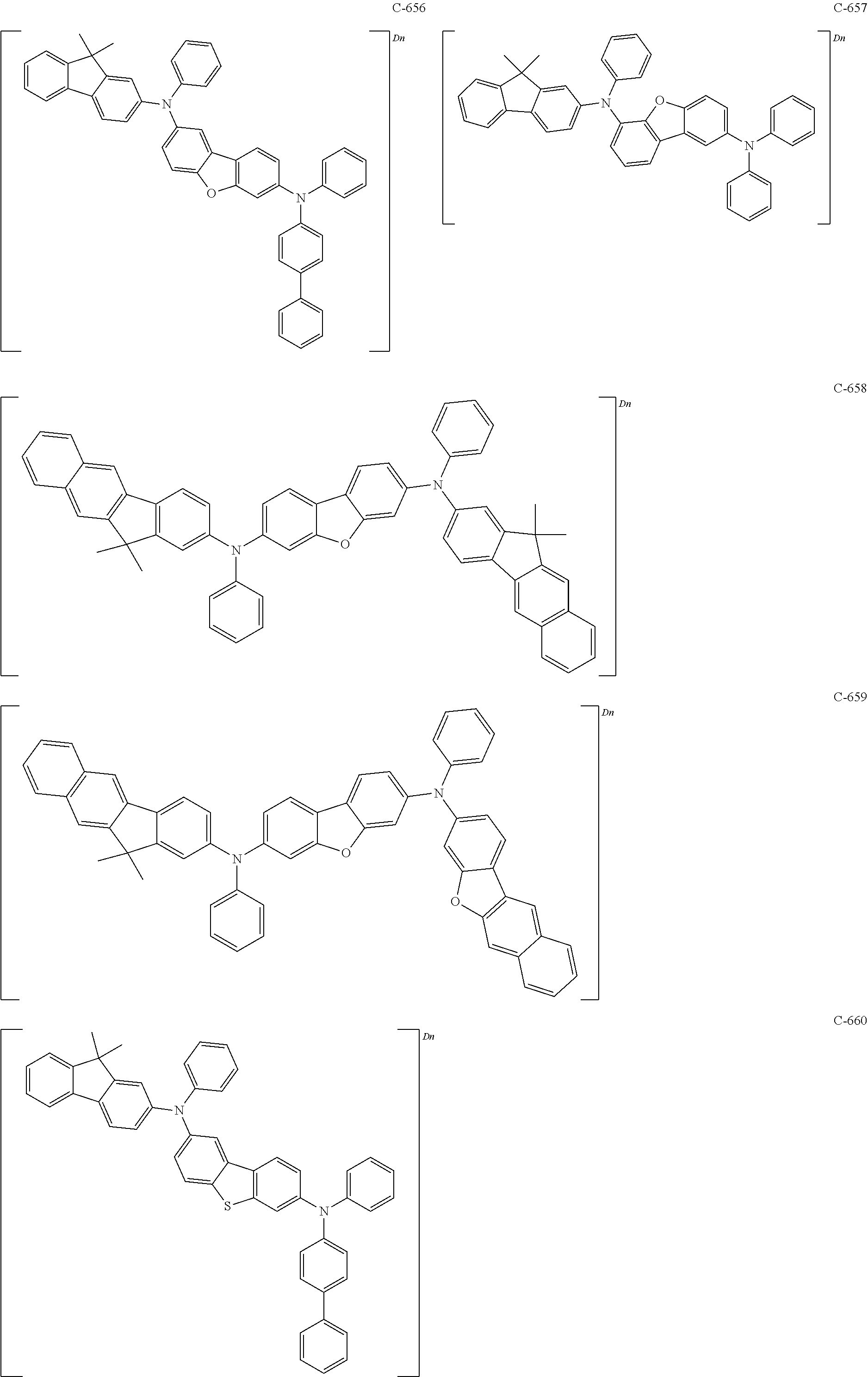

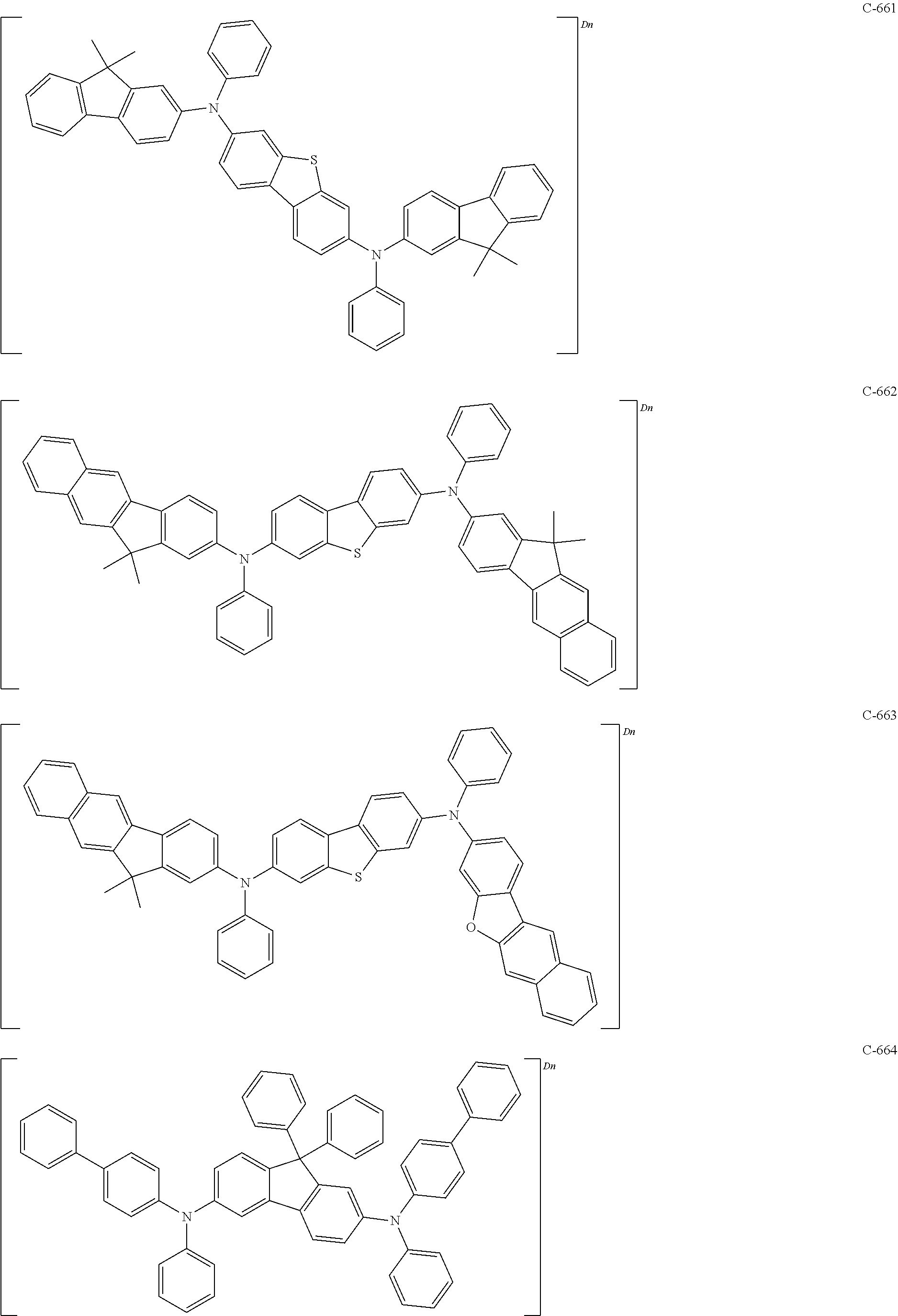

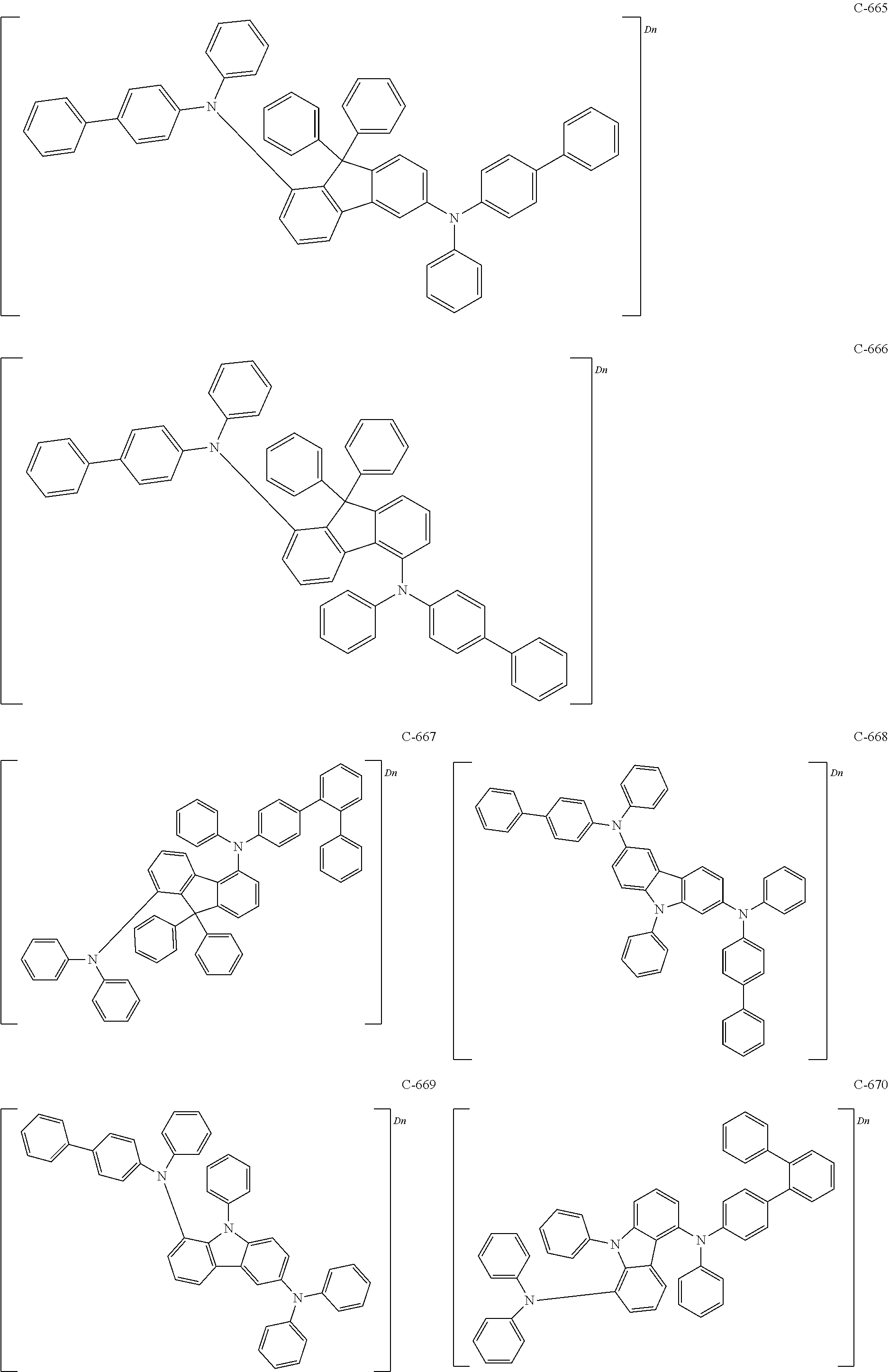

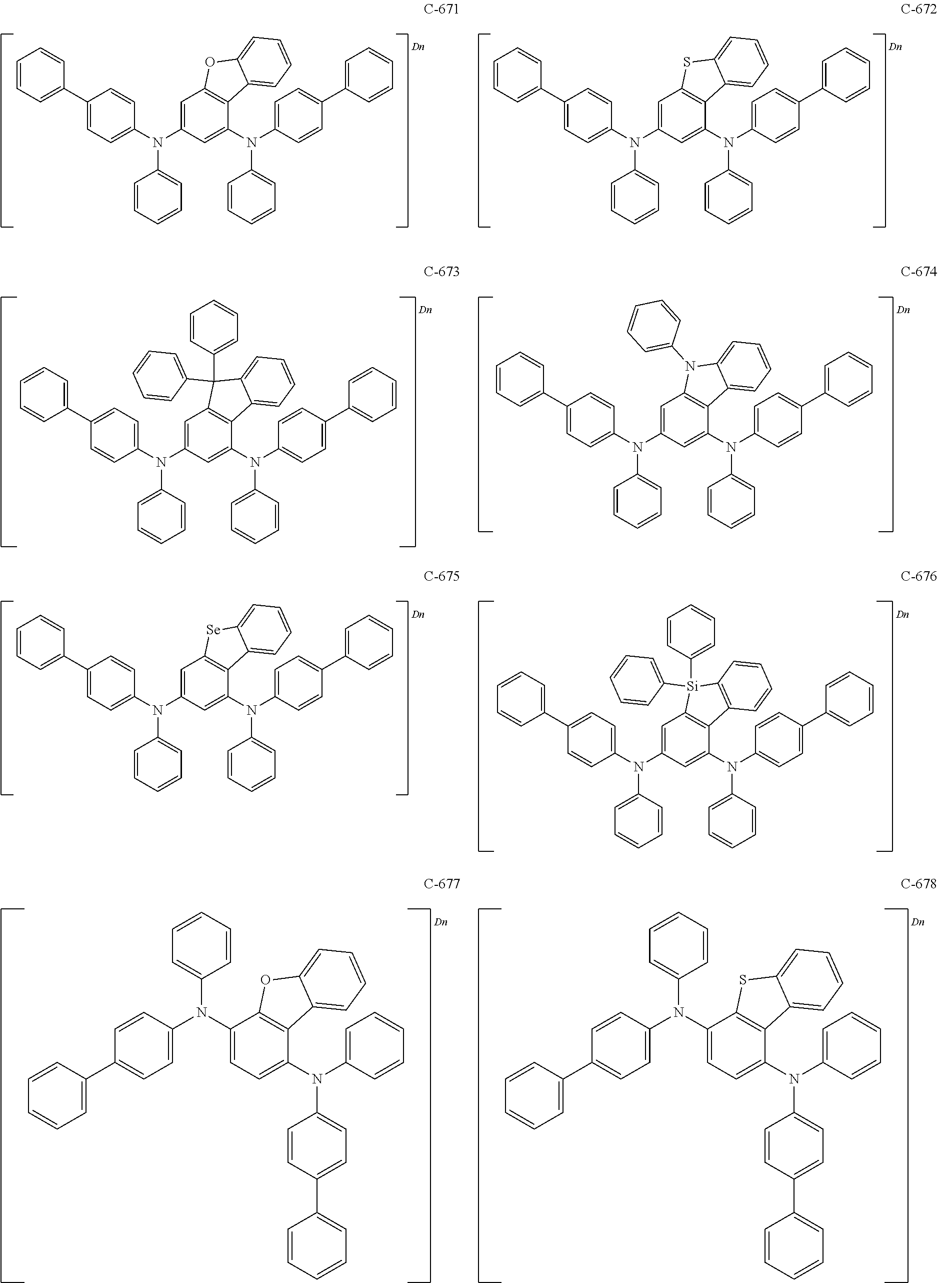

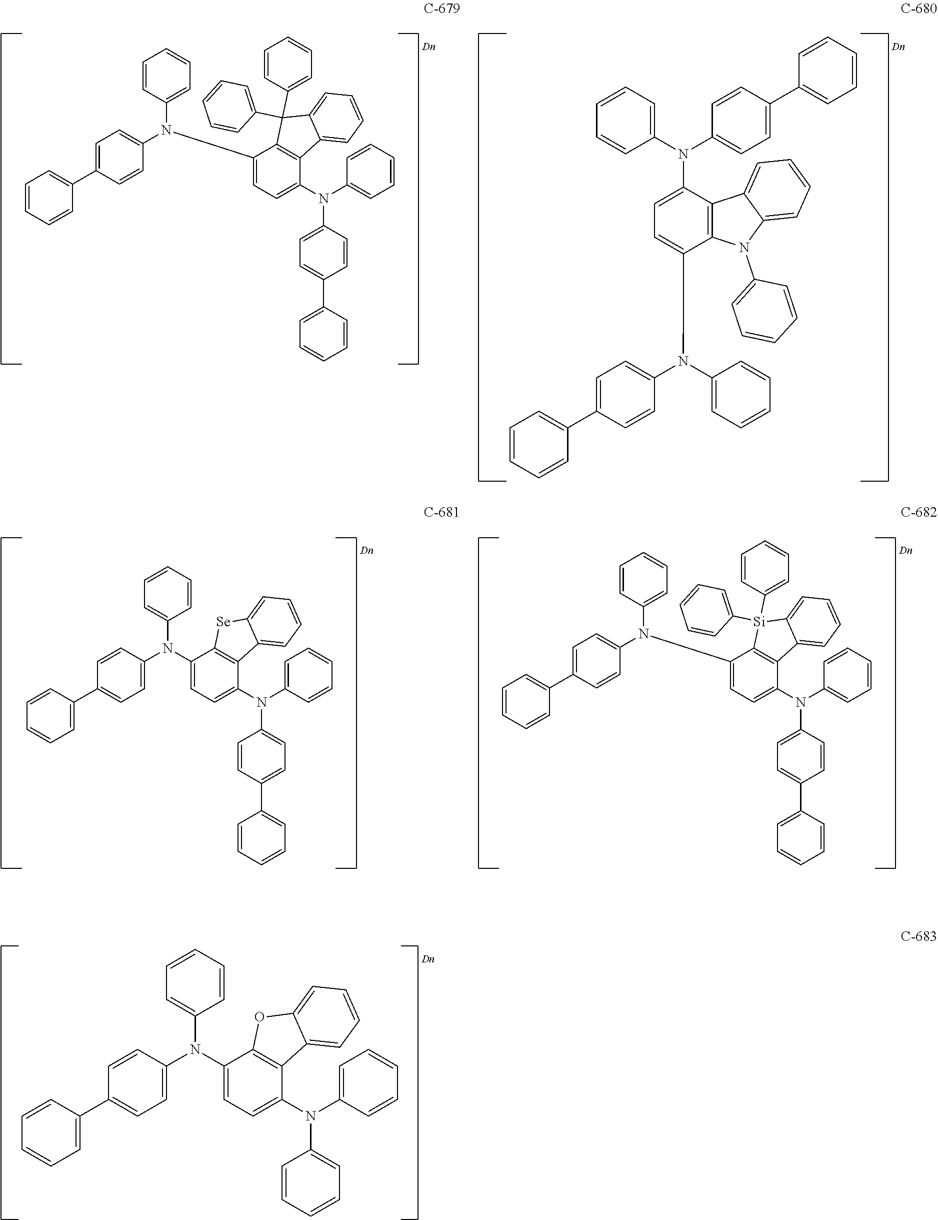

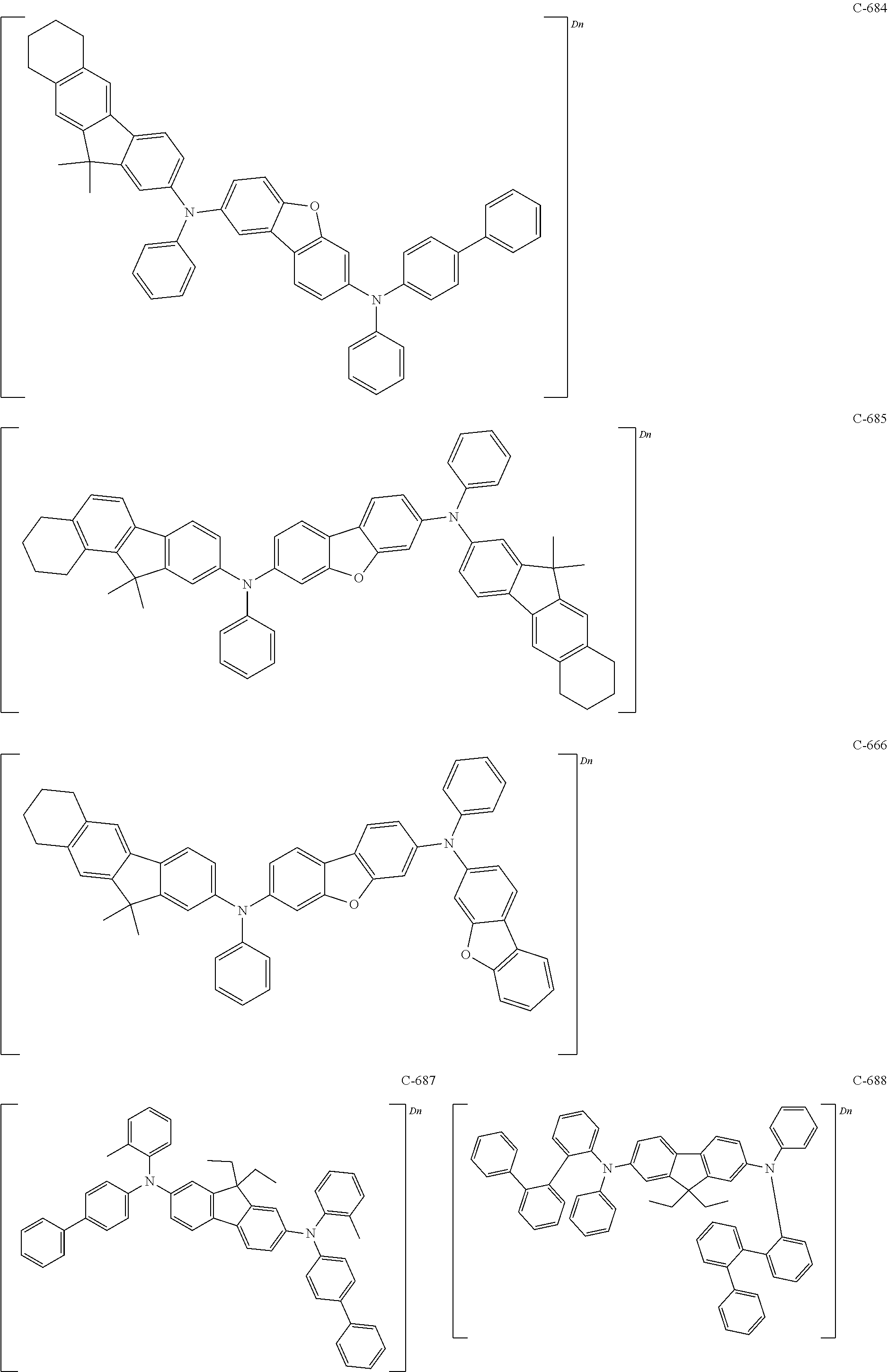

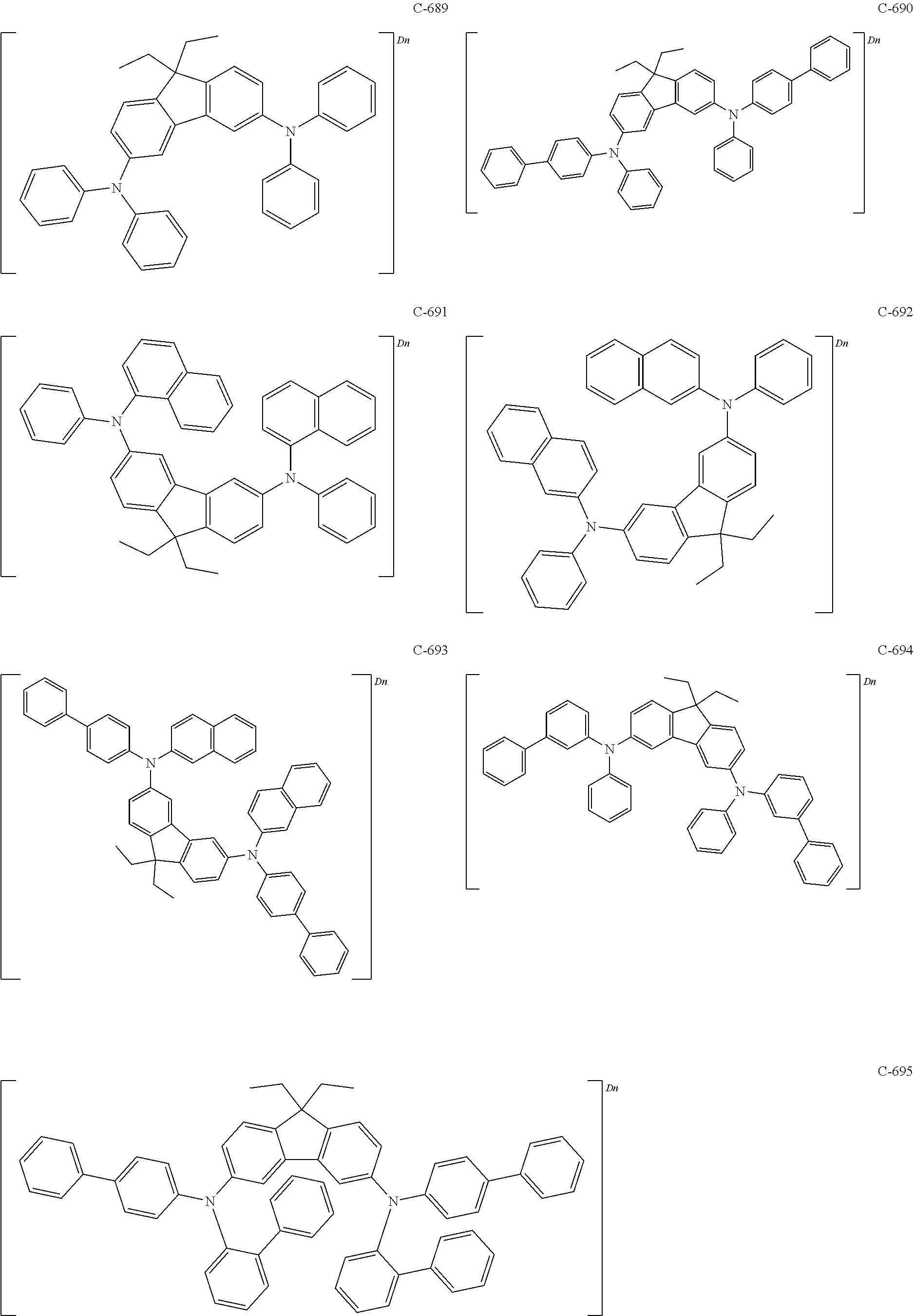

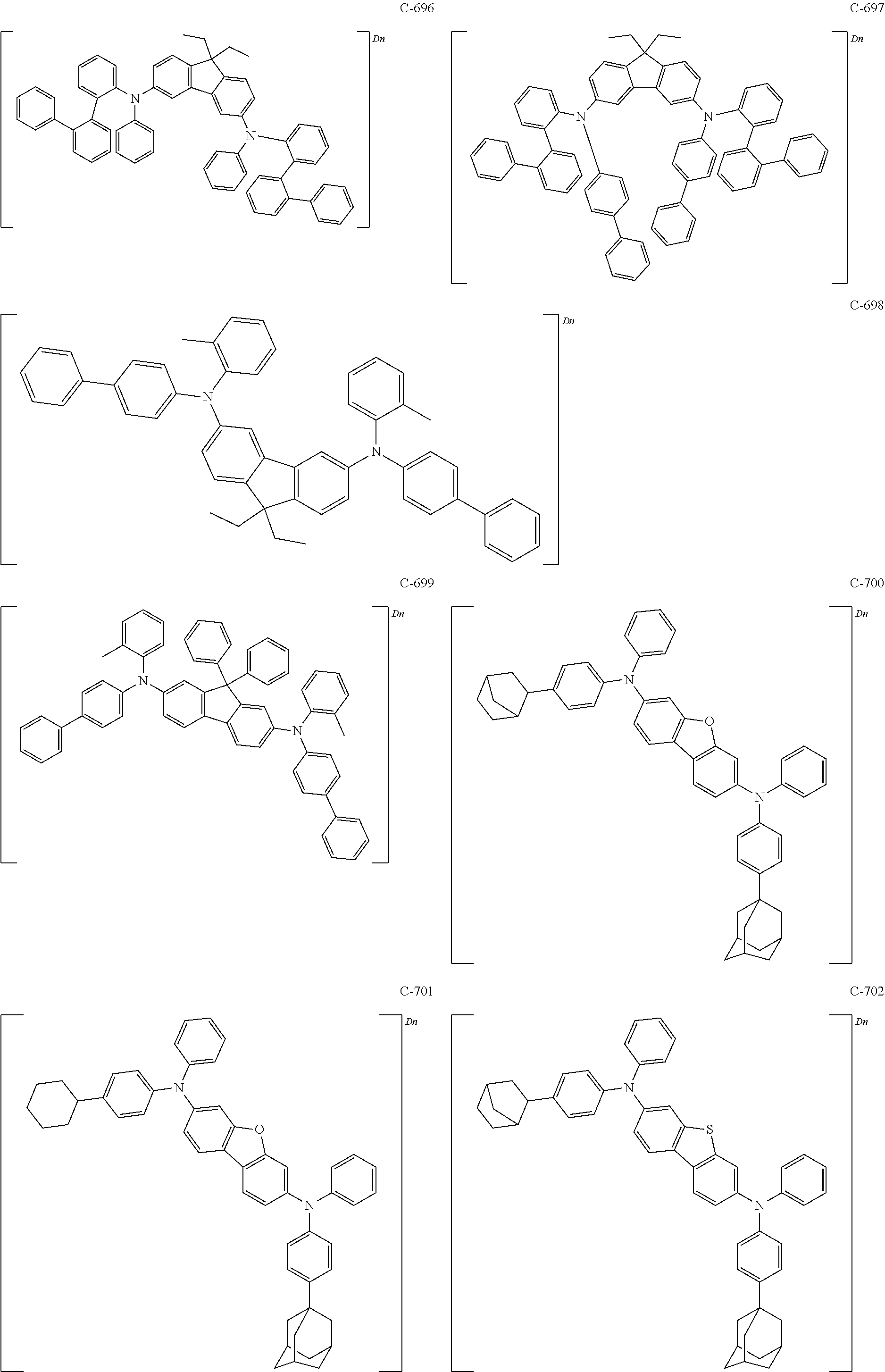

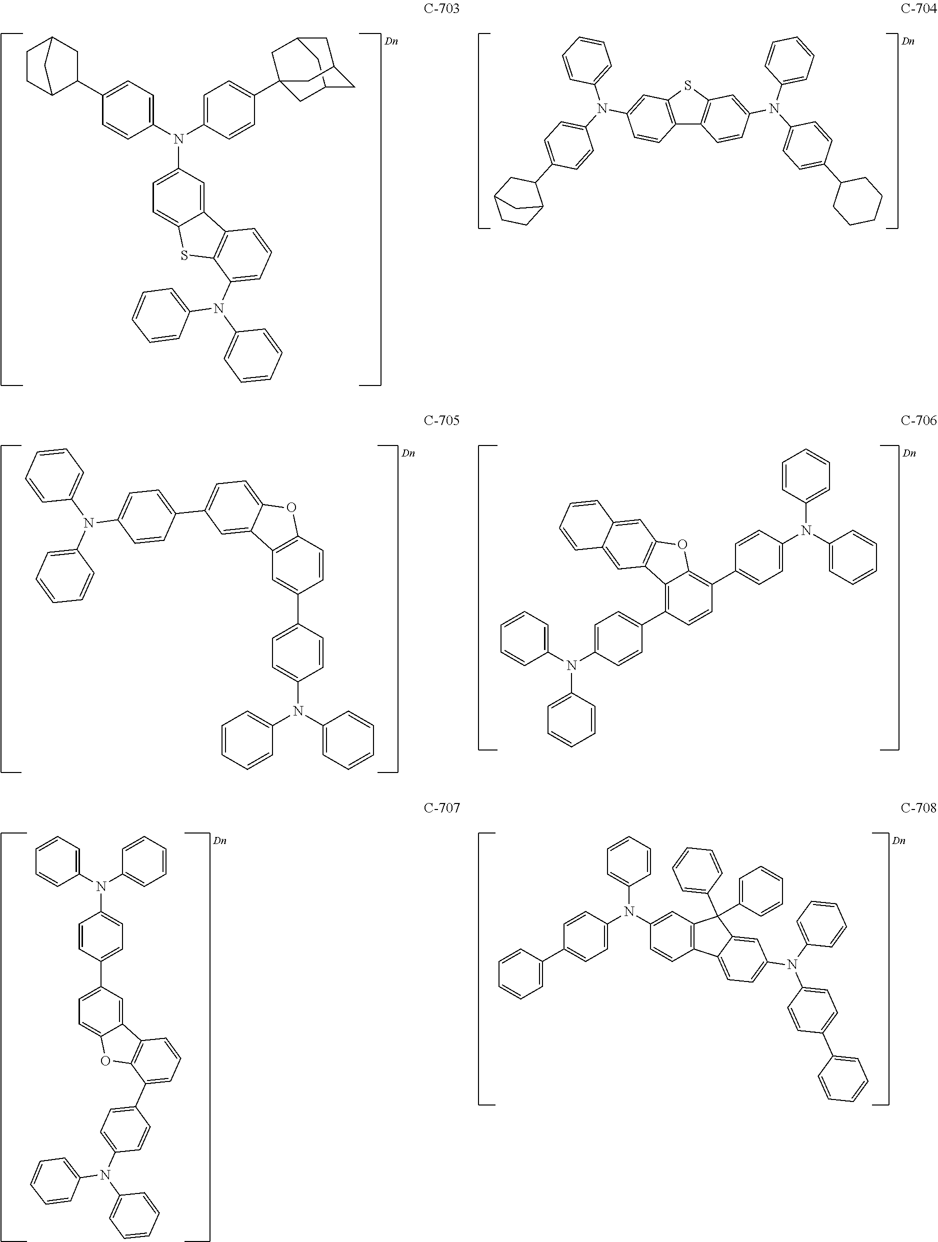

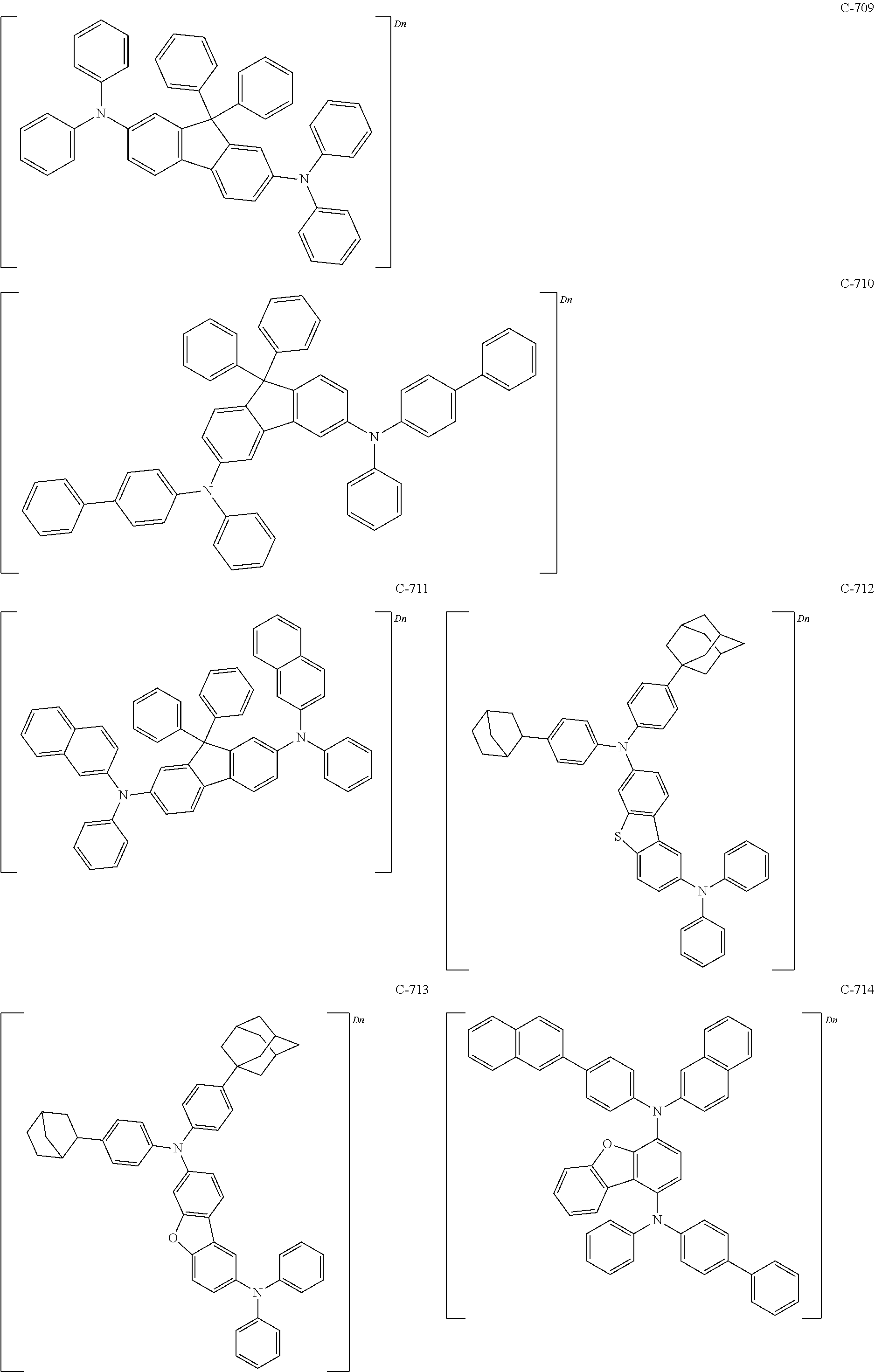

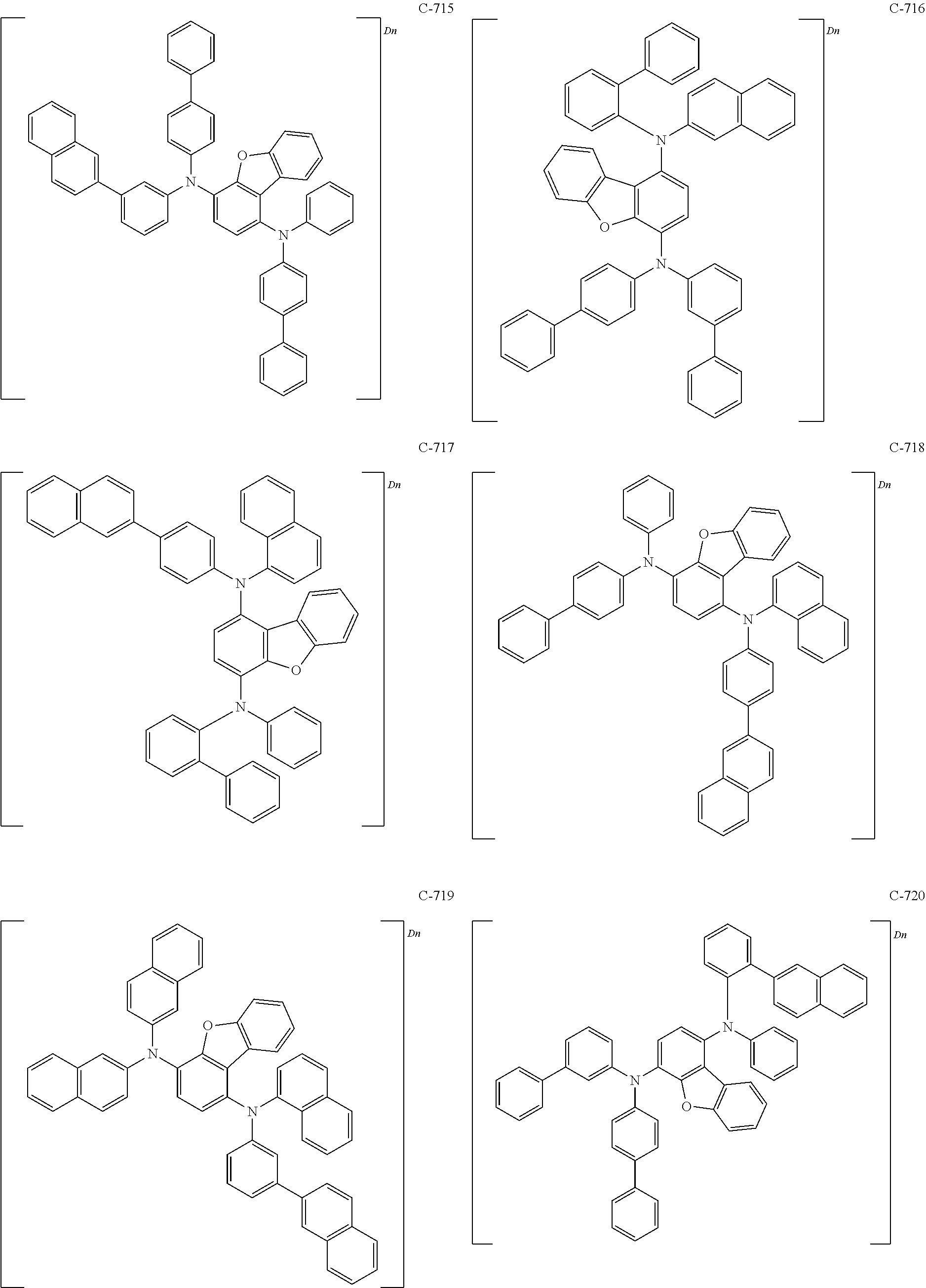

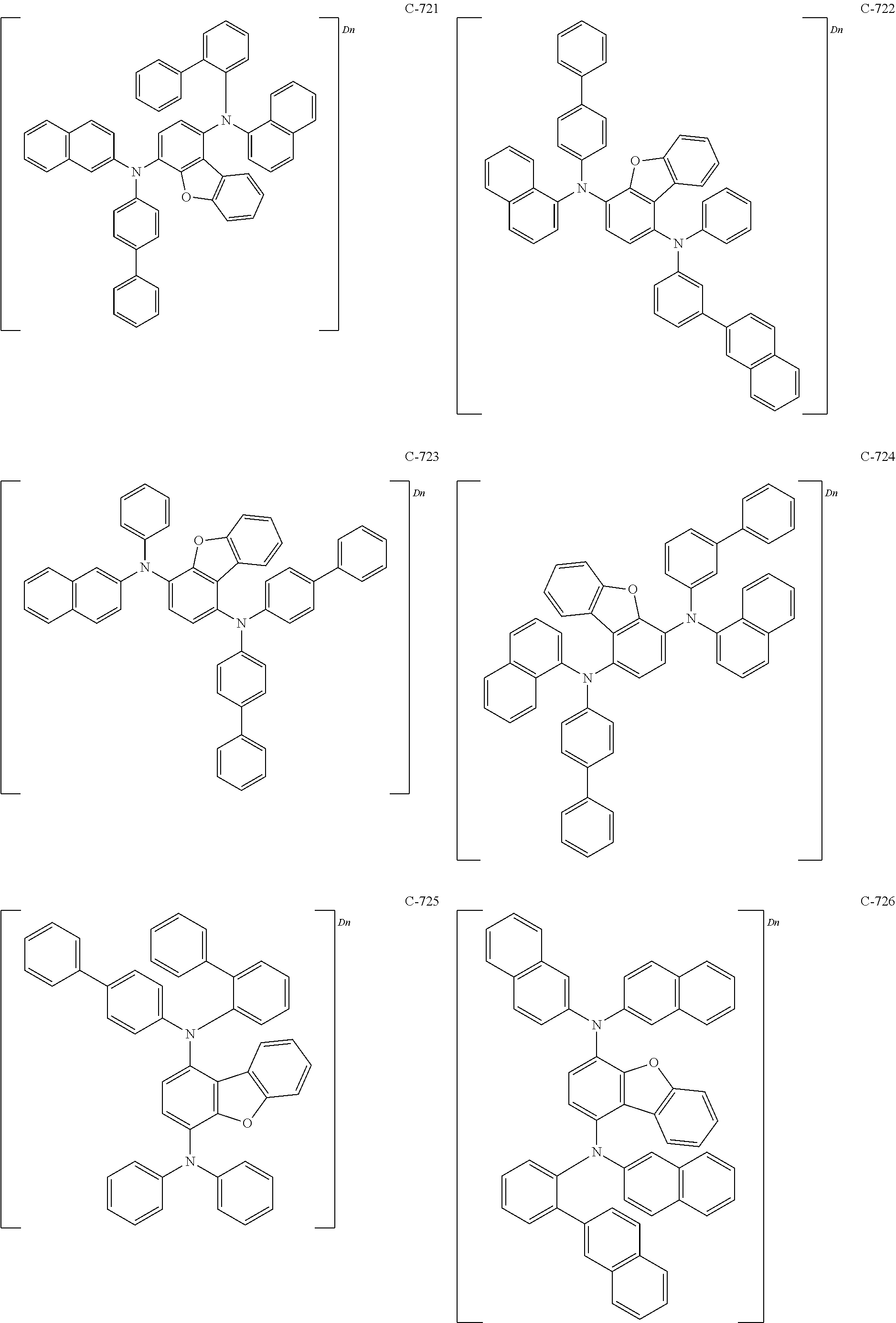

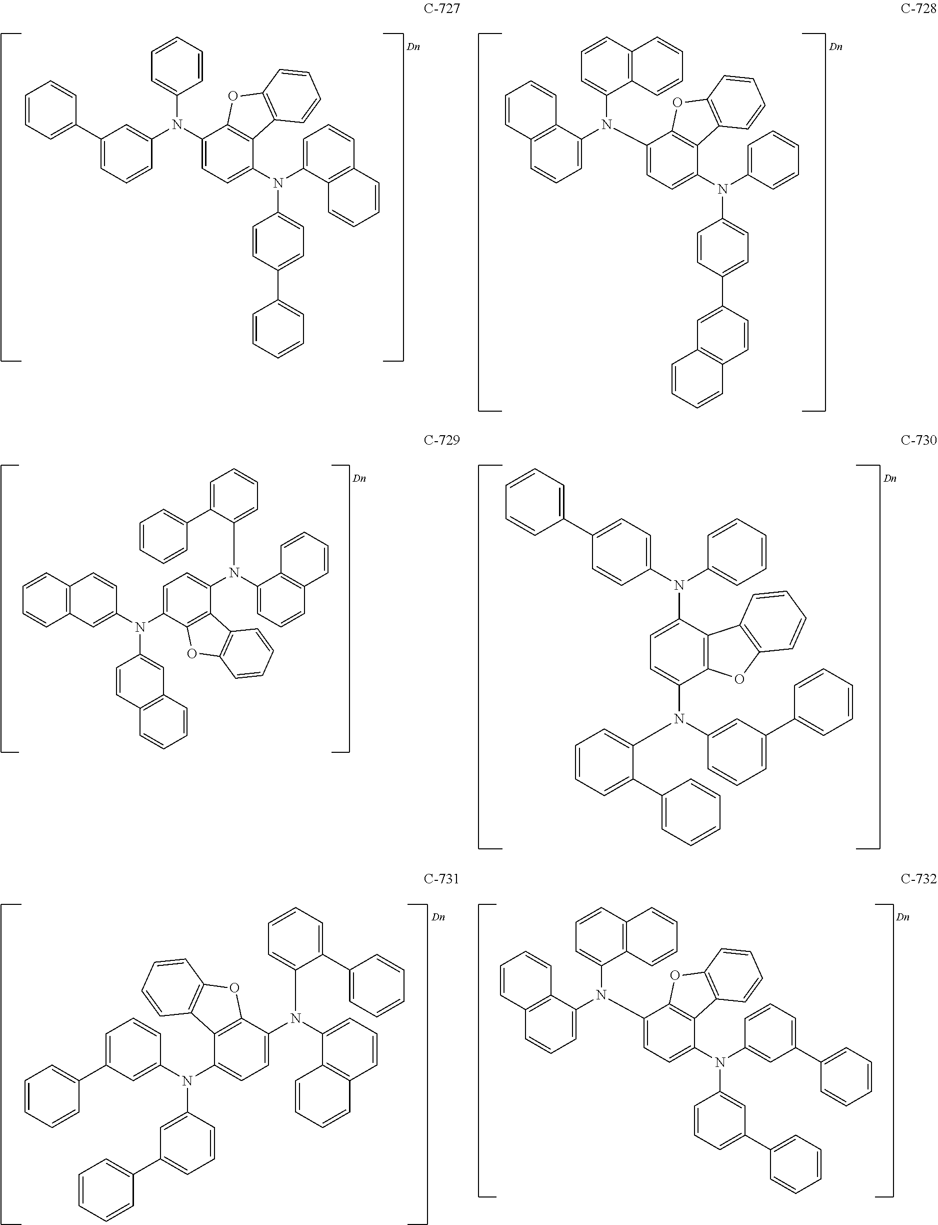

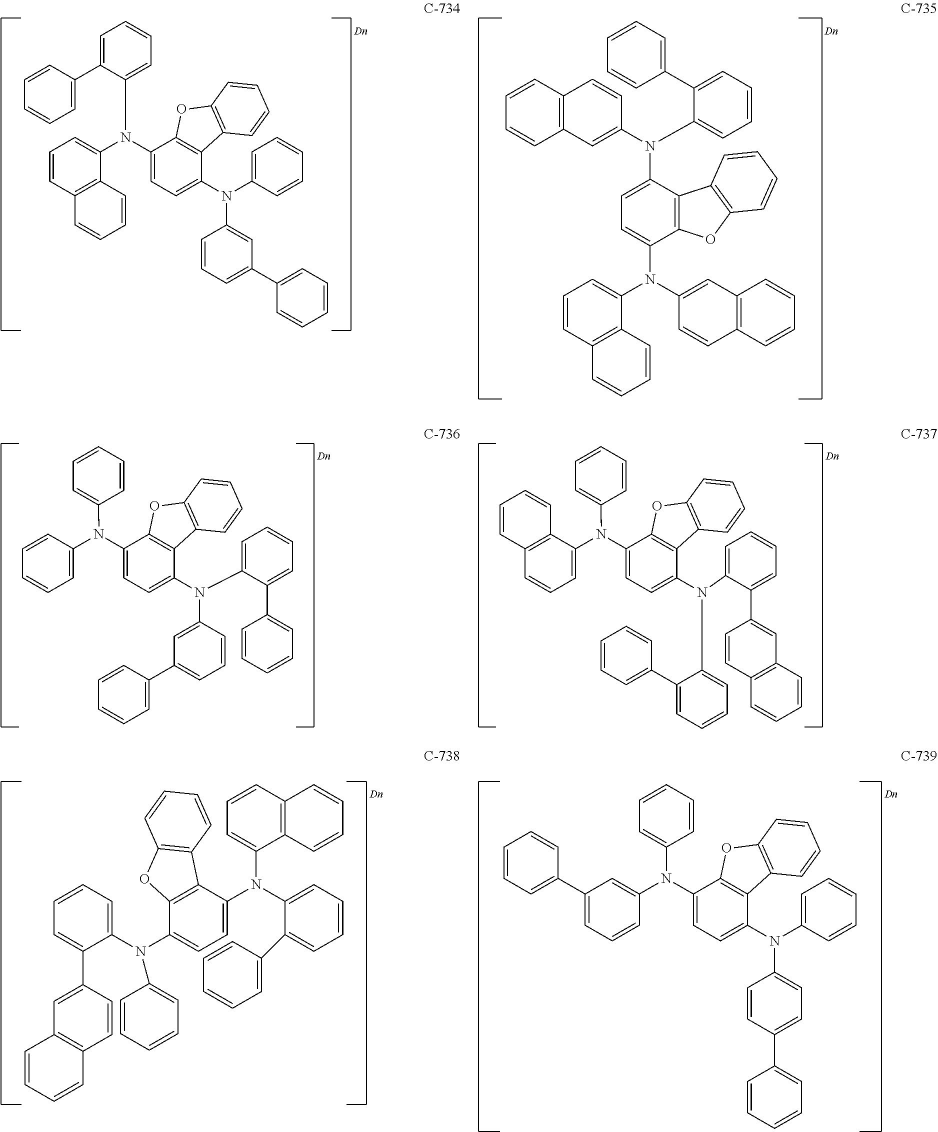

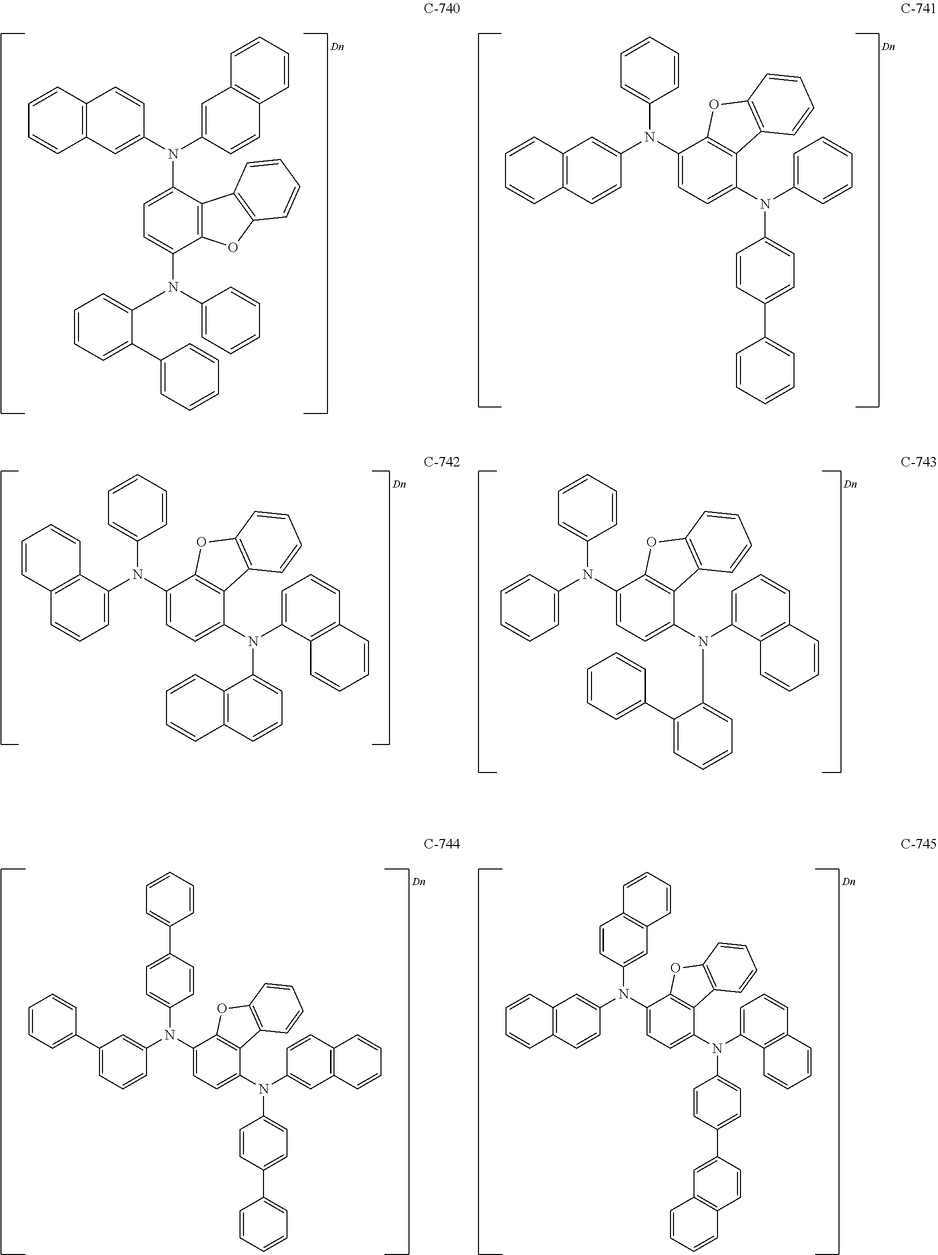

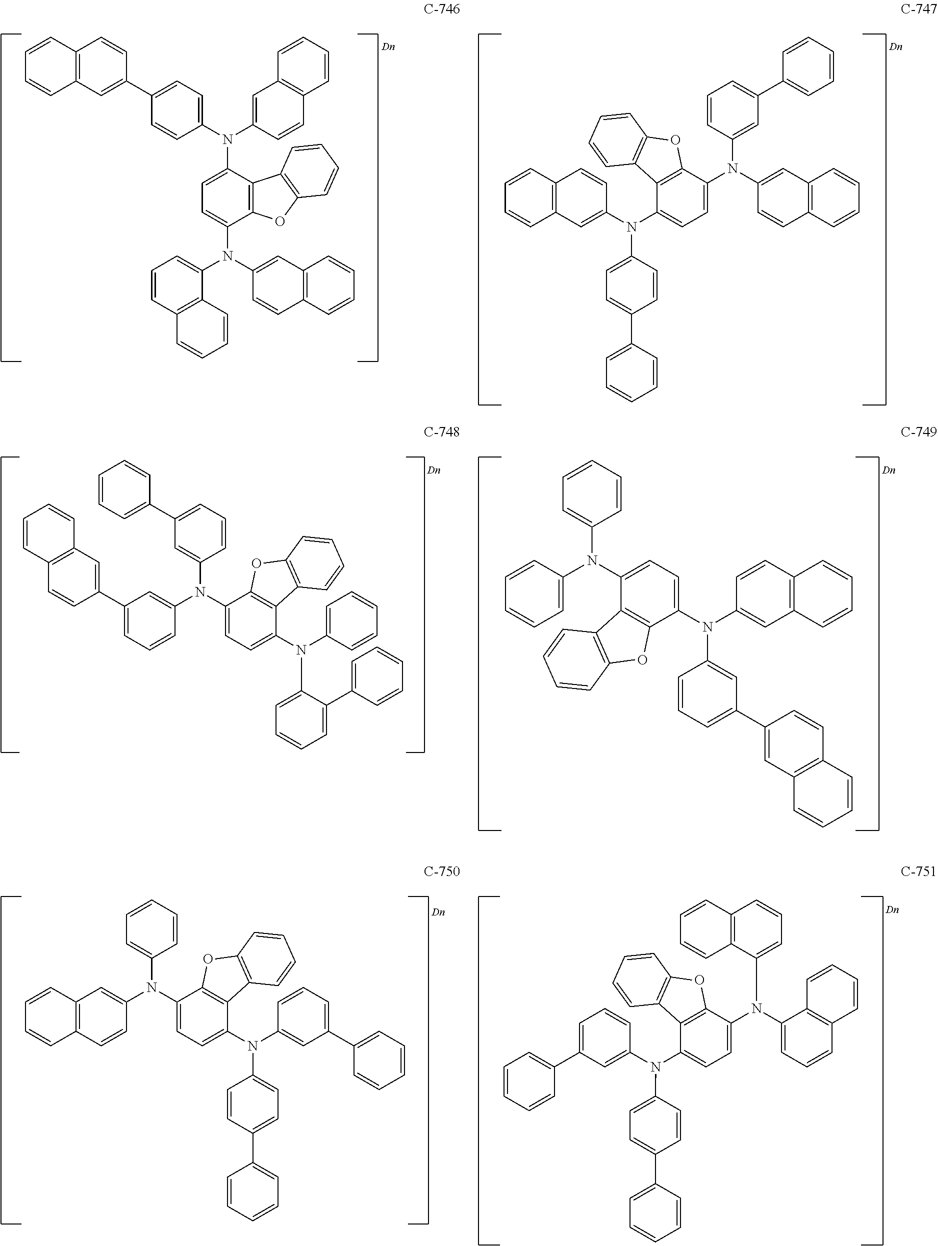

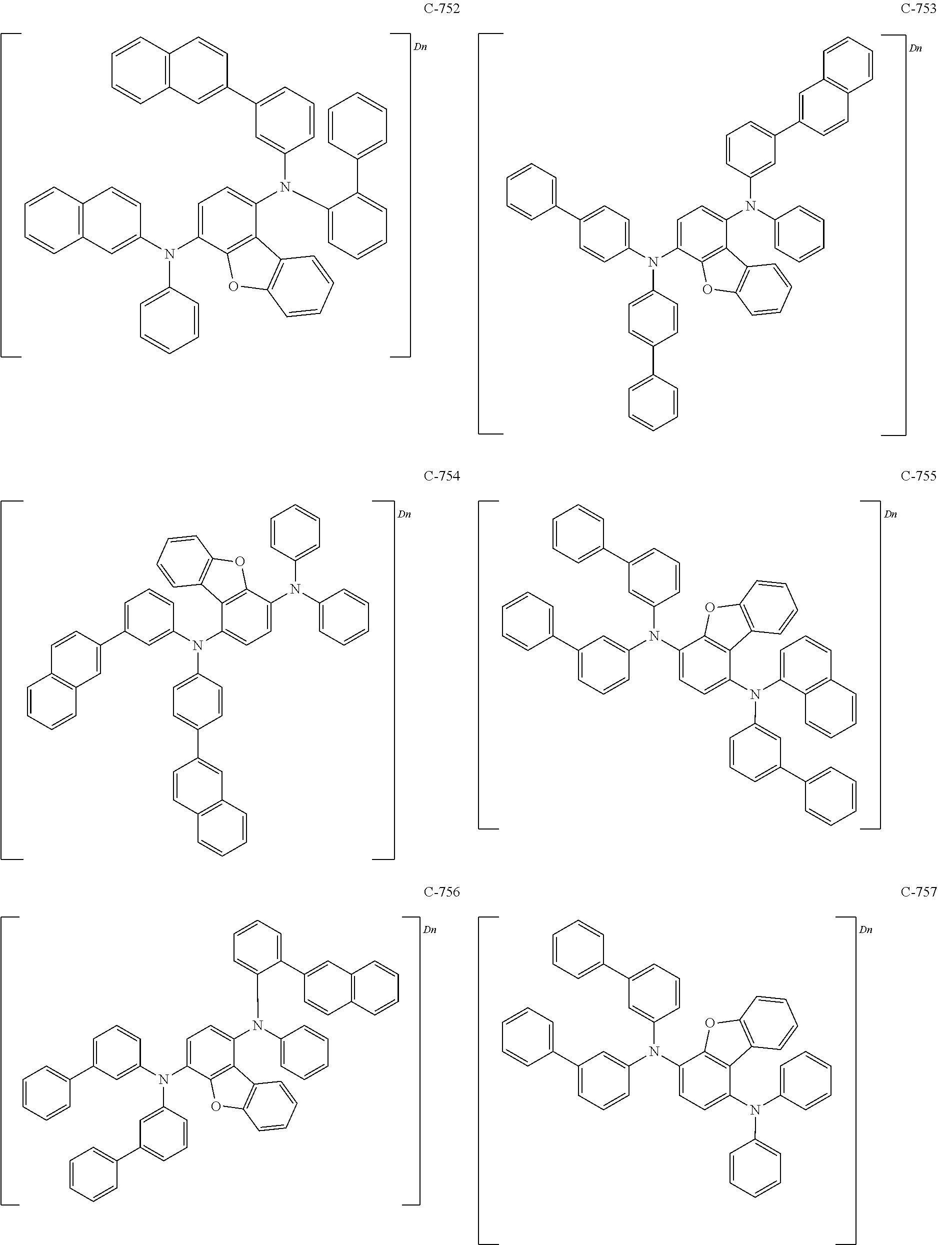

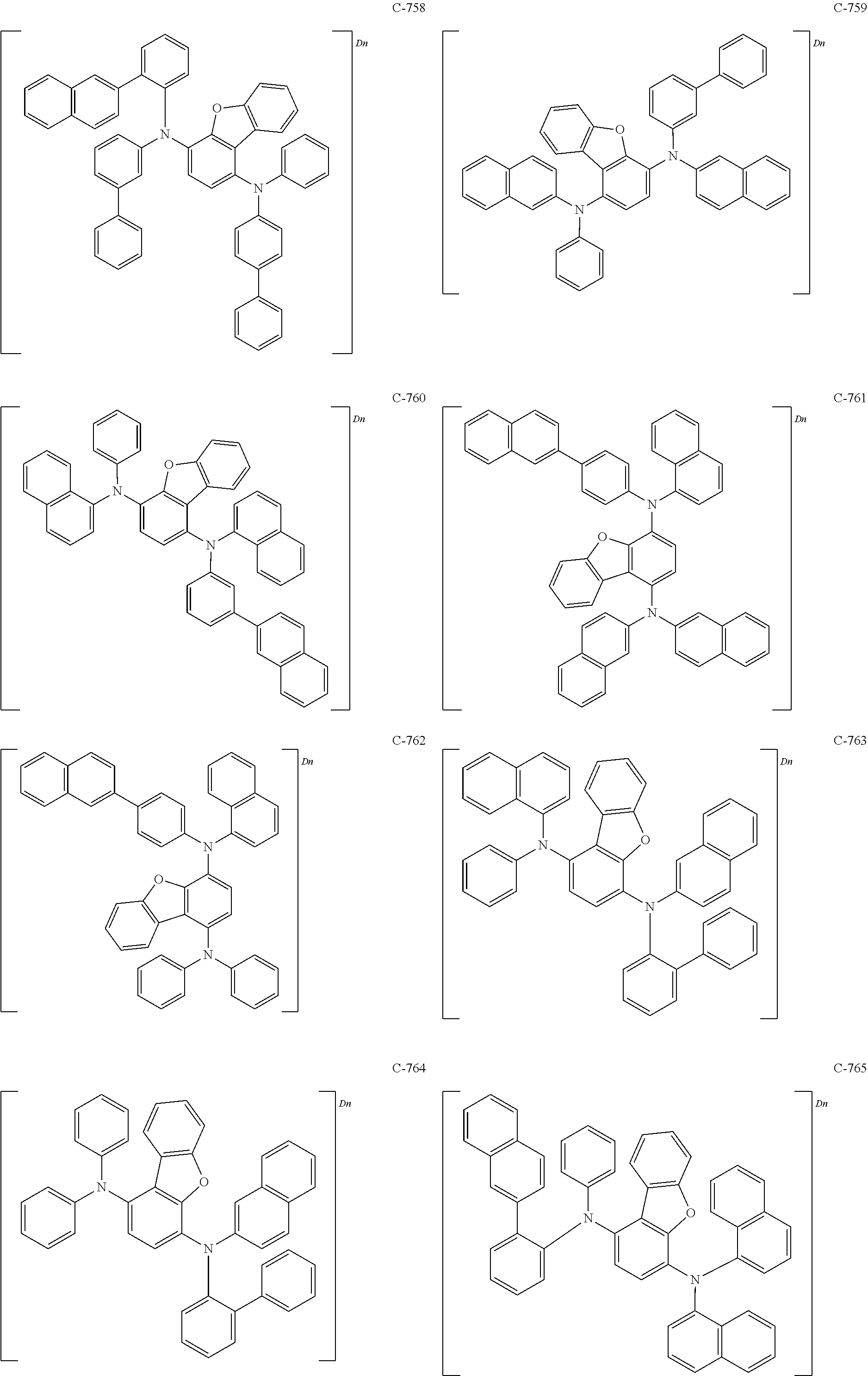

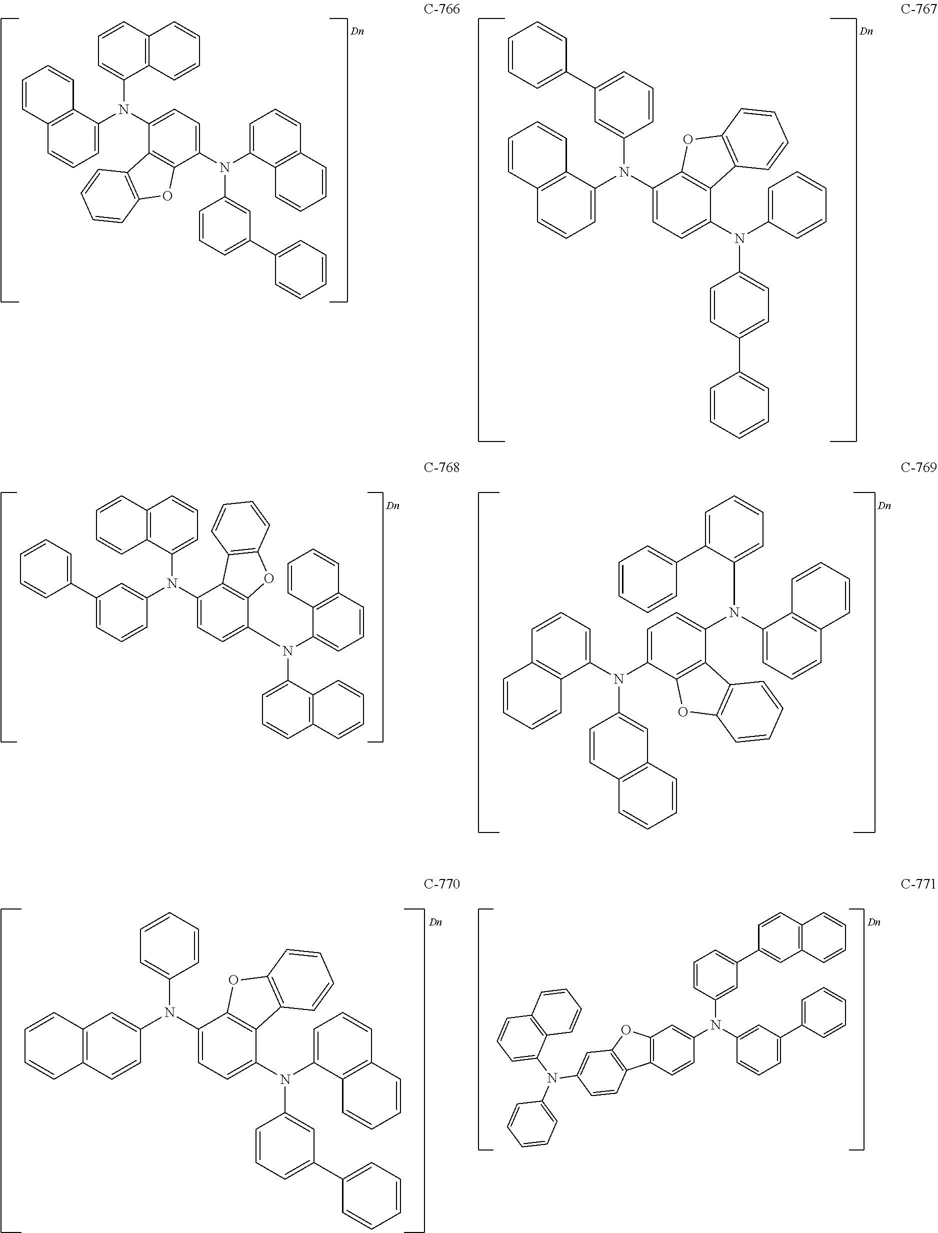

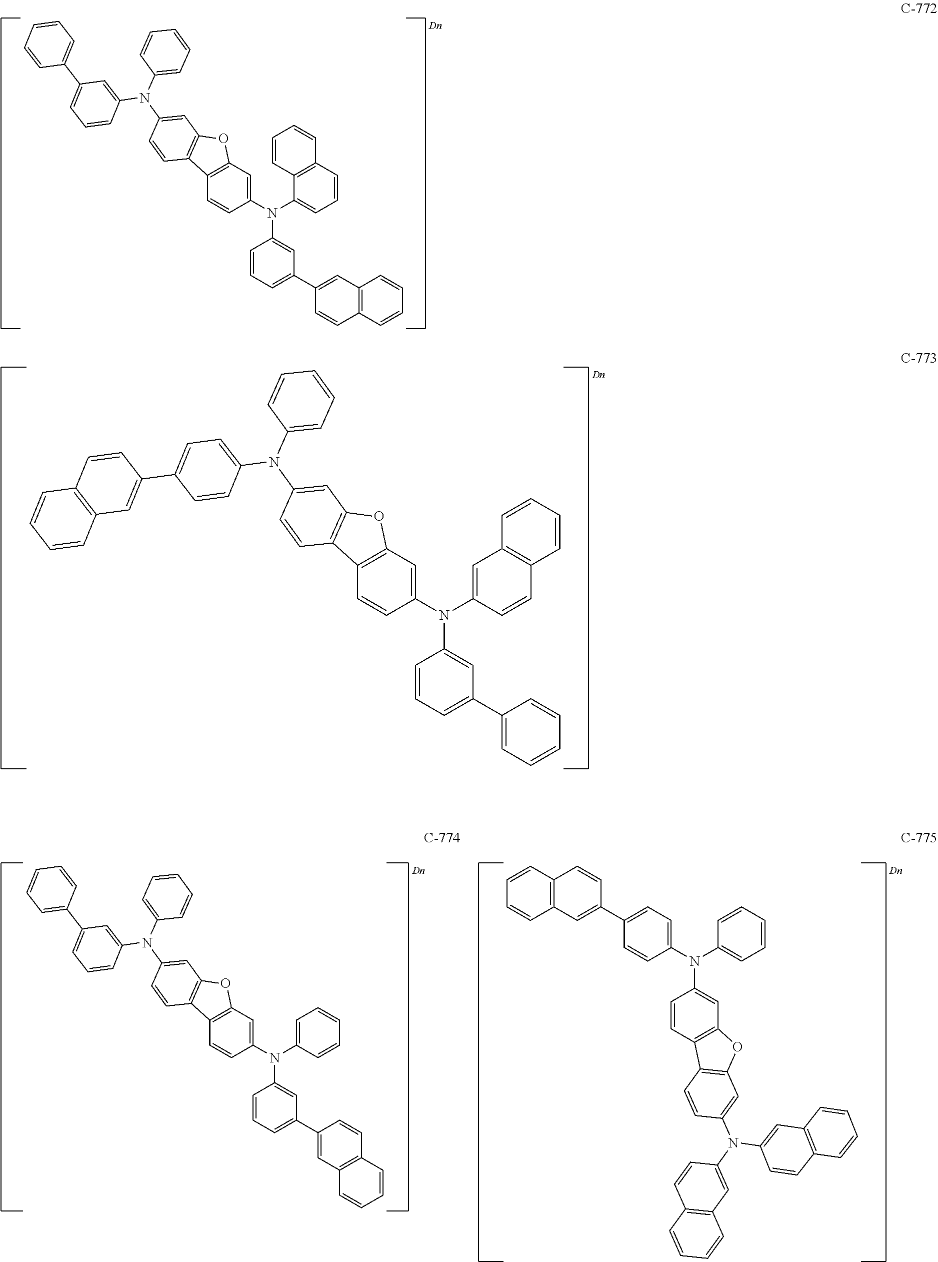

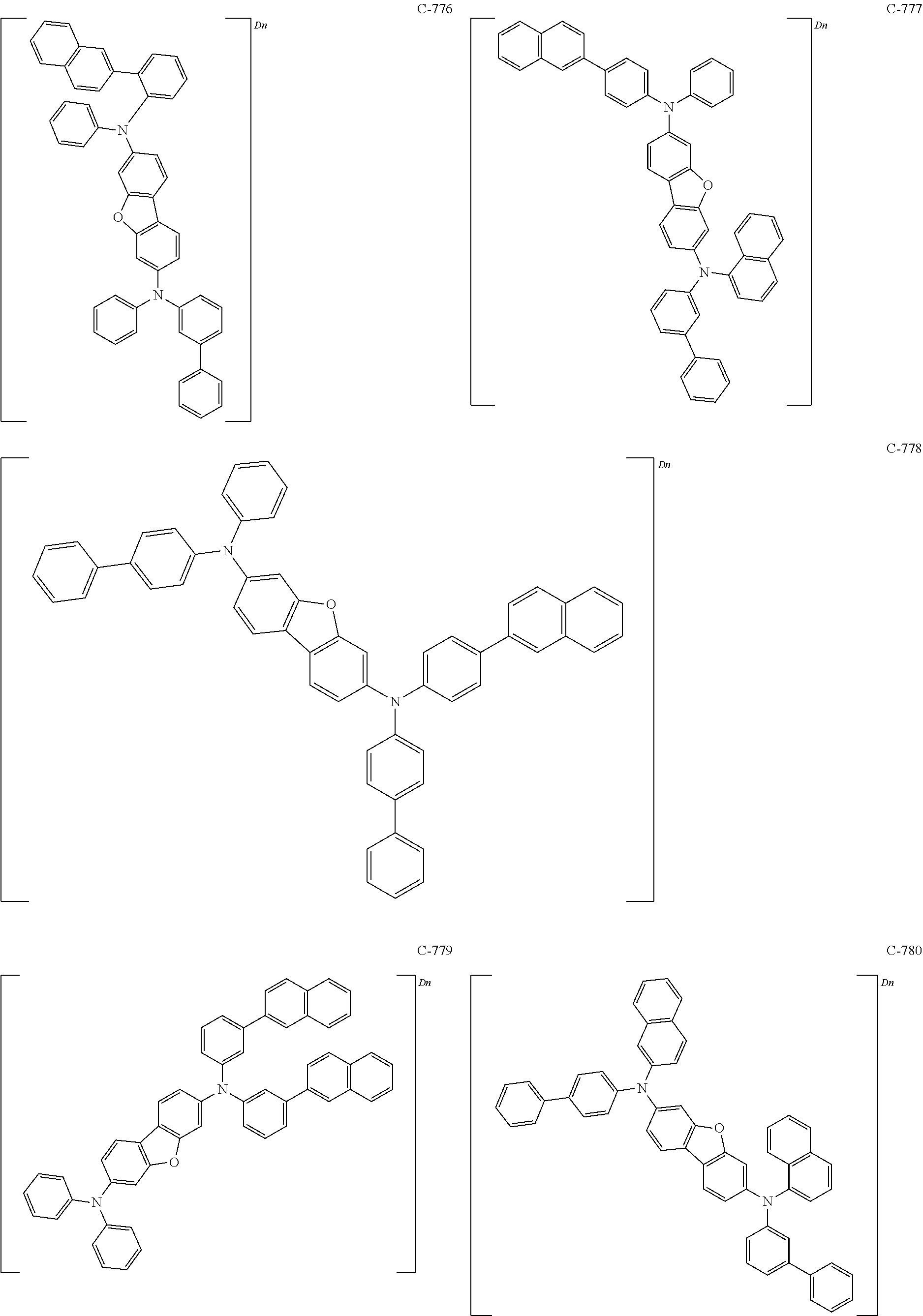

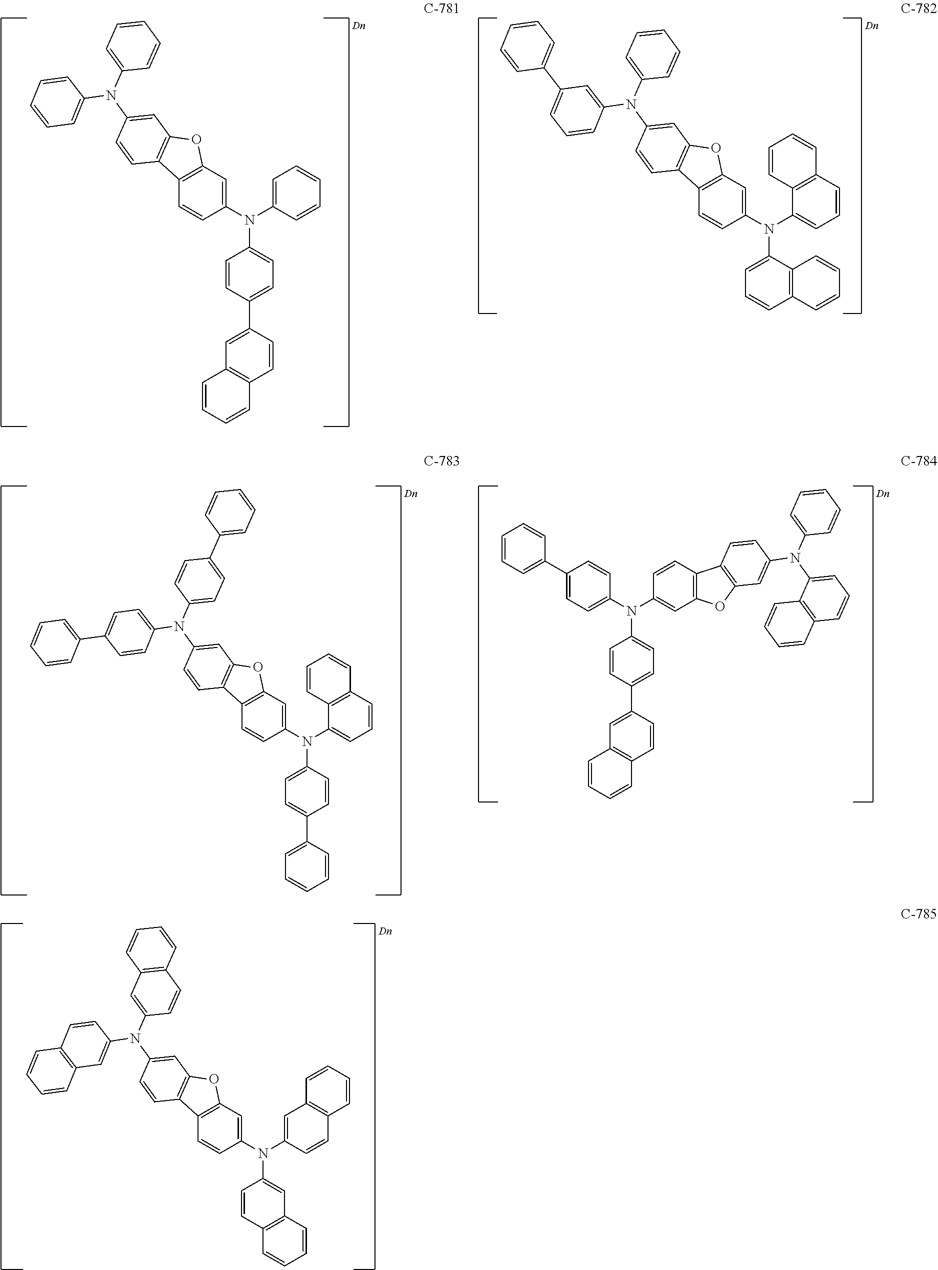

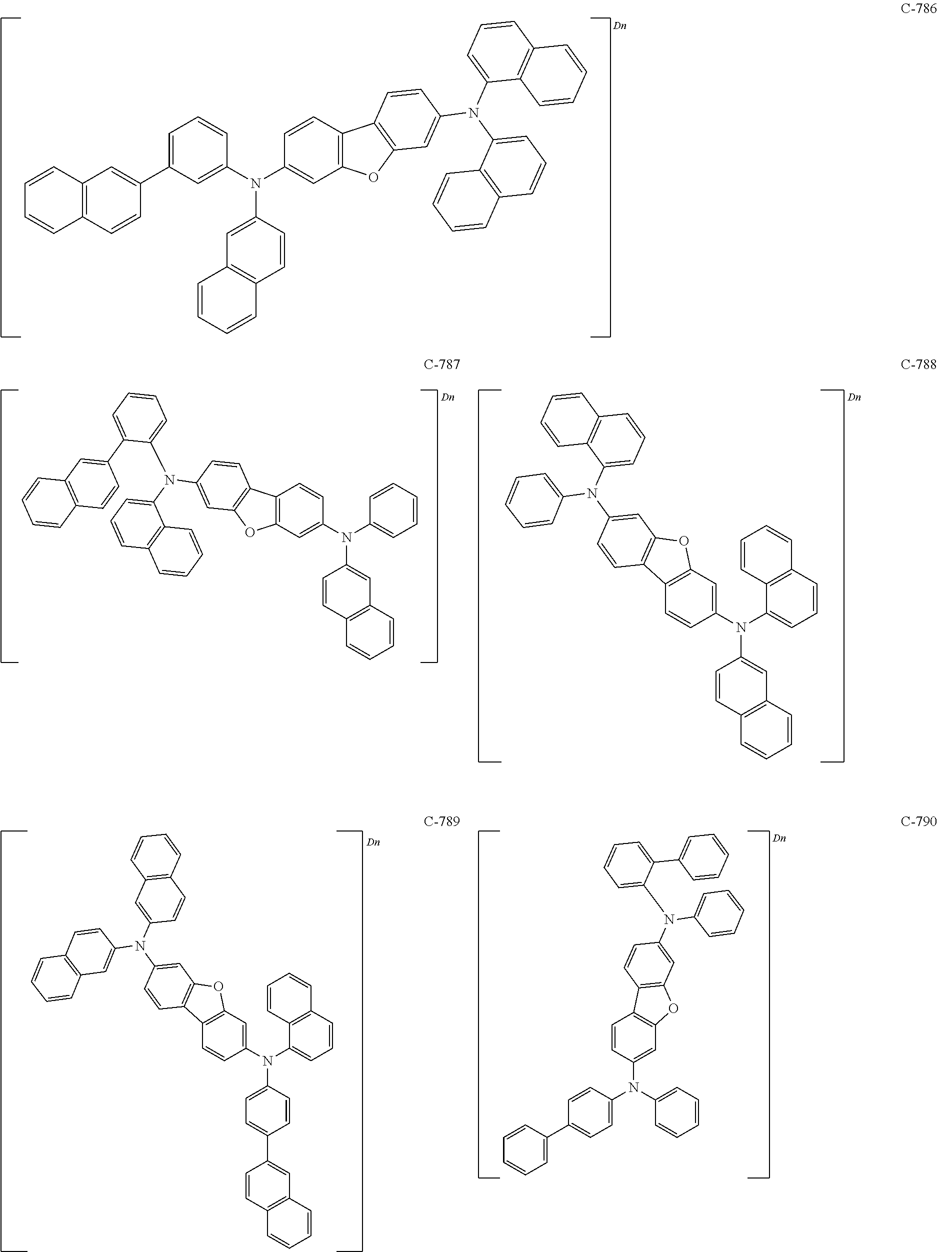

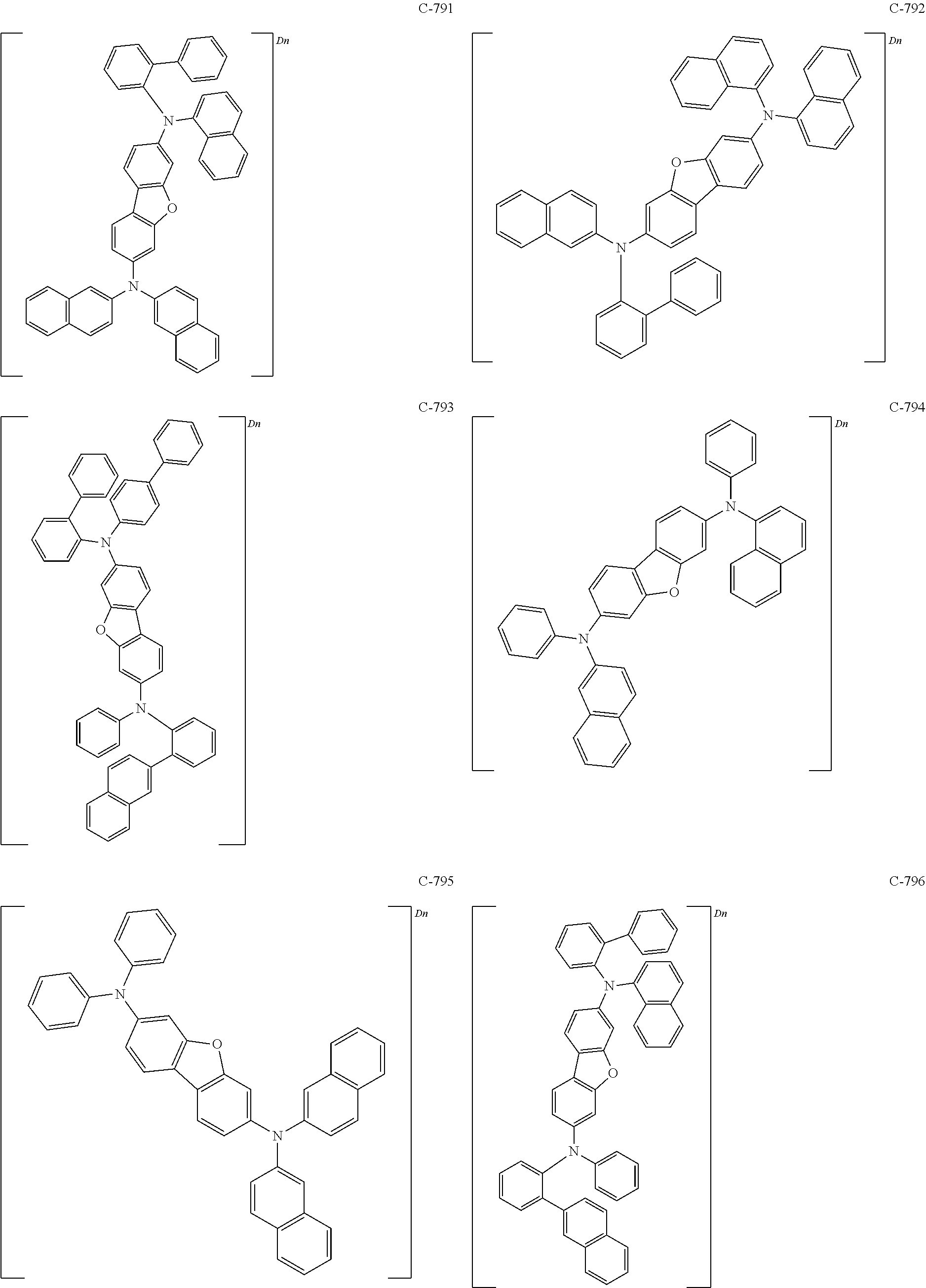

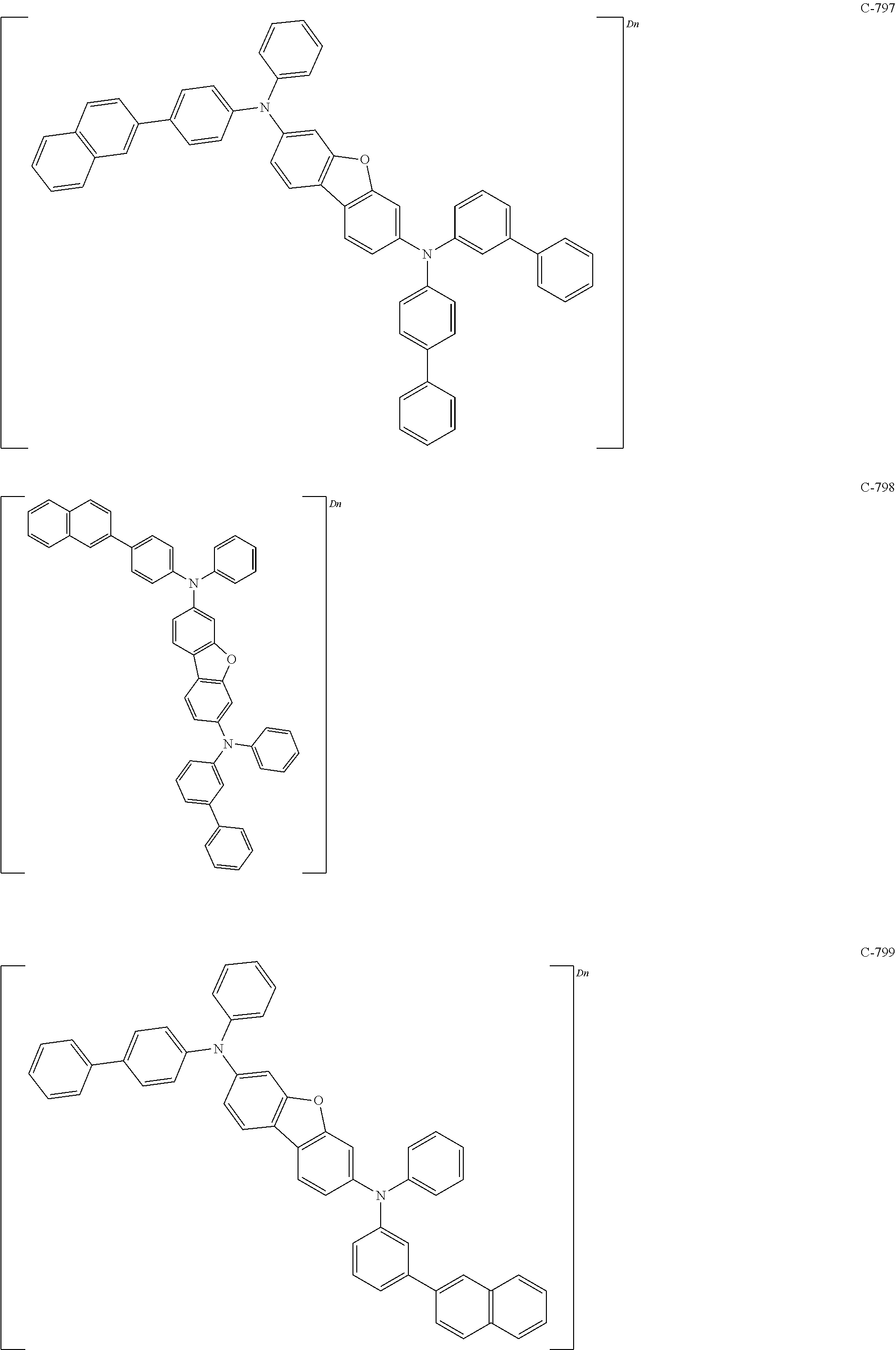

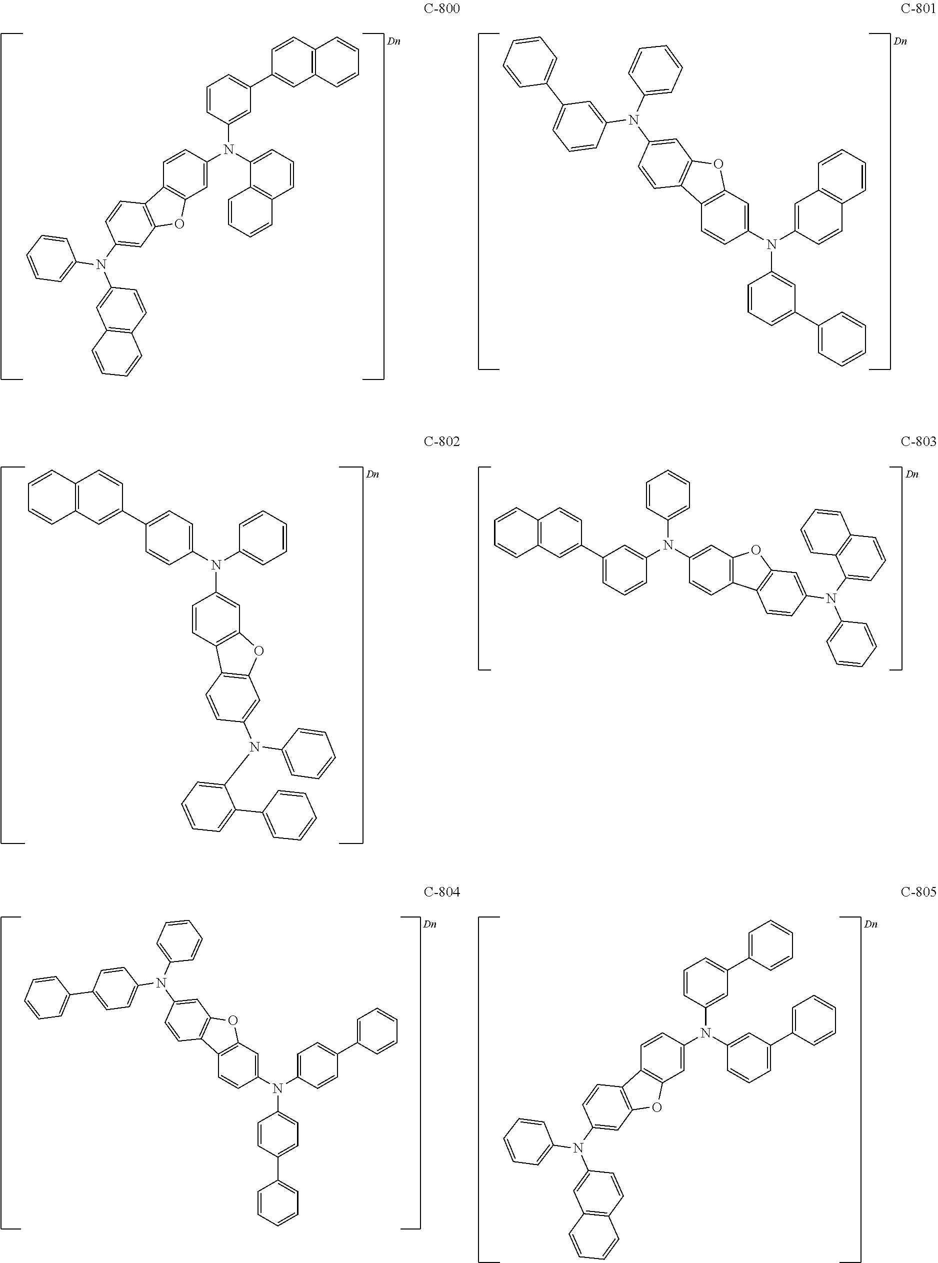

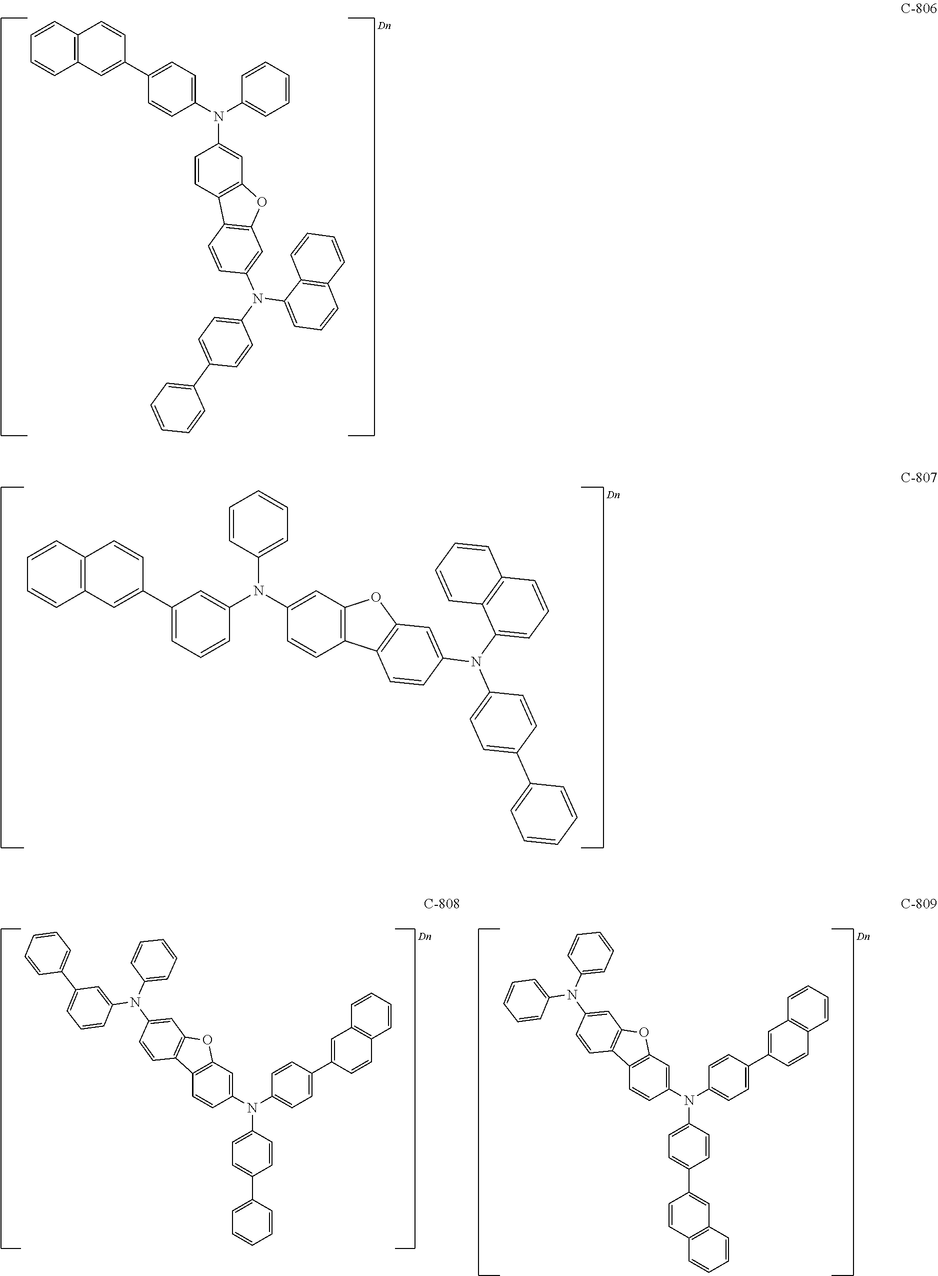

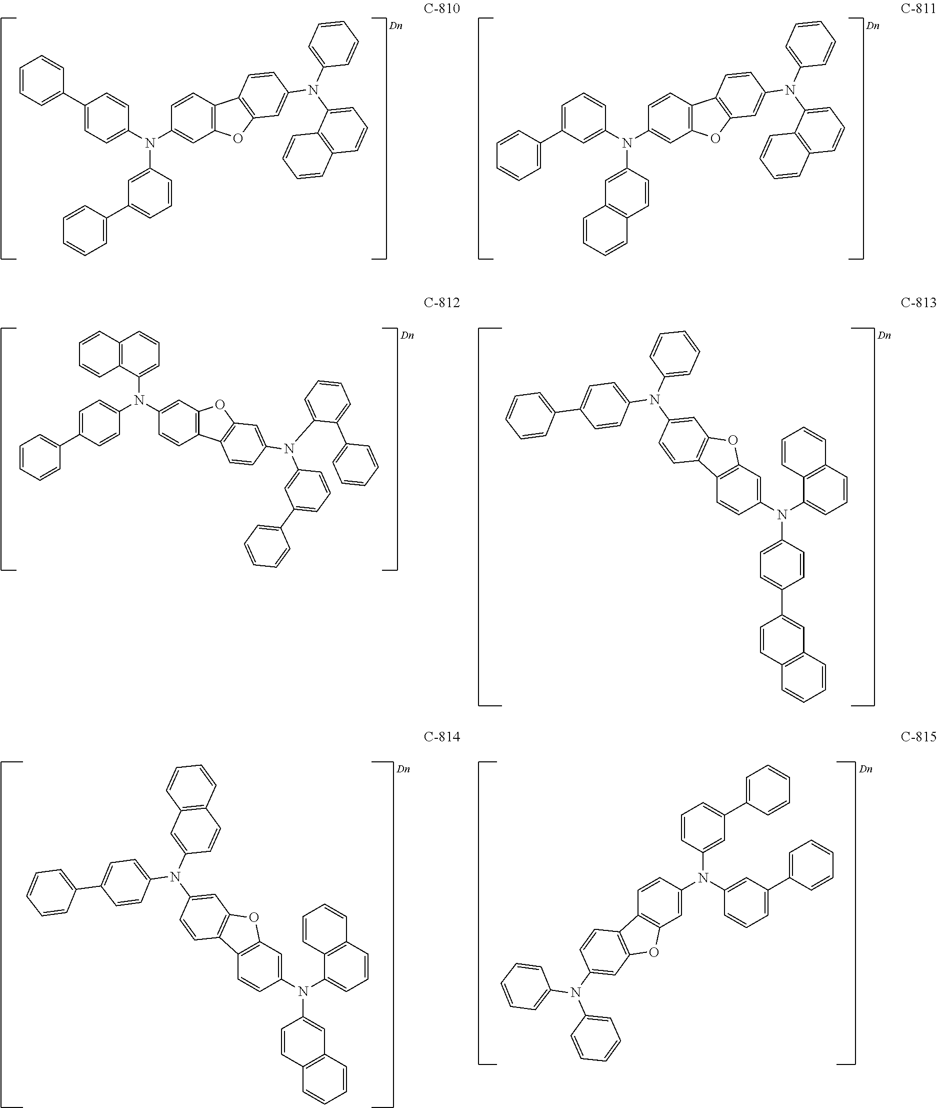

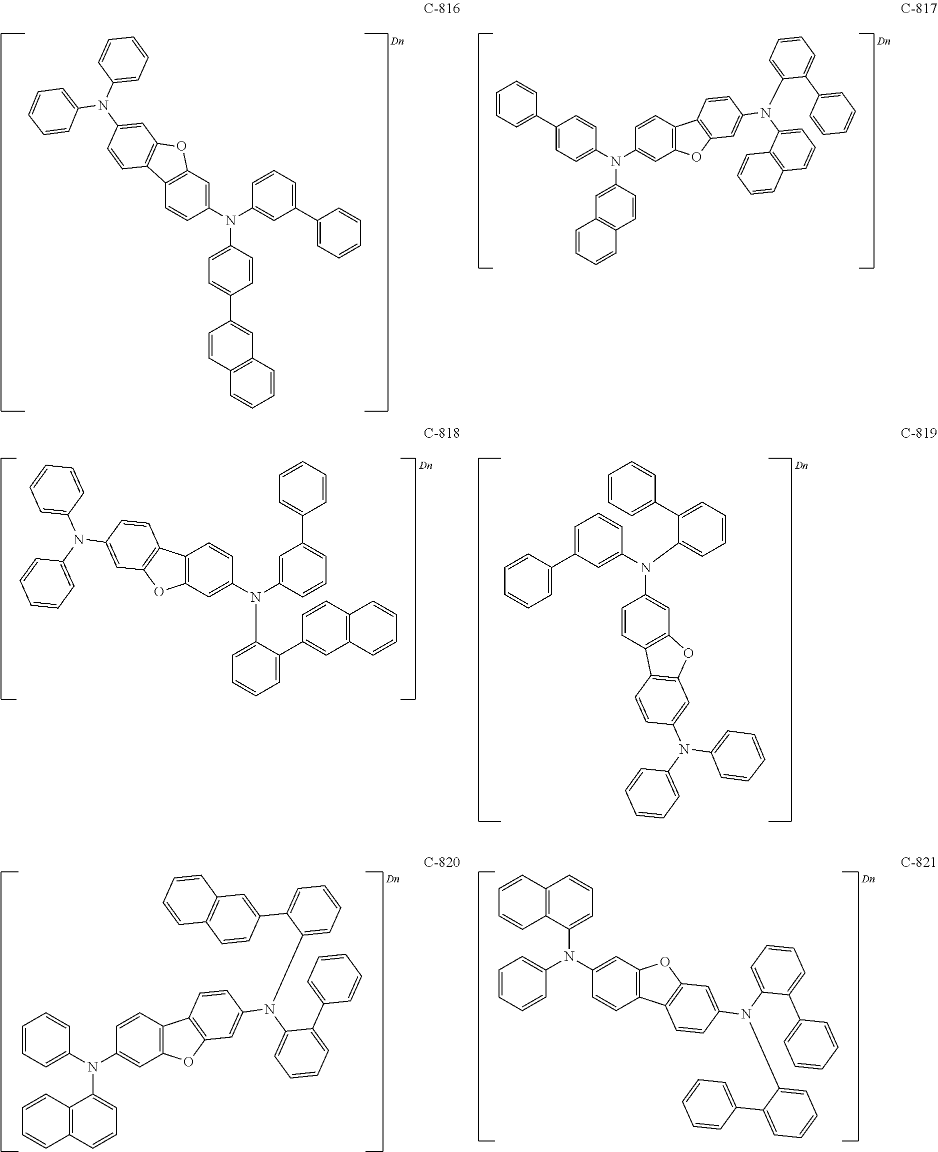

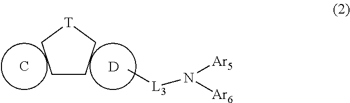

In the compounds above, Dₙ means that n number of hydrogens are replaced with deuterium, and n represents an integer from 1 to the maximum number of hydrogens in the compound.

According to one embodiment of the present disclosure, in an organic electroluminescent device, at least one of the plurality of hole transport layers may comprise a compound represented by the following Formula 2.

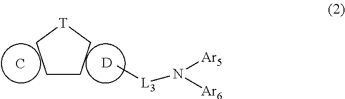

In Formula 2,
T represents —N(R₅₁)—, —C(R₅₂)(R₅₃)—, —Si(R₅₄)(R₅₅)—, —Se—, —O—, or —S—;
ring C or ring D each independently represents a substituted or unsubstituted (C₆-C₃₀)arene, or a substituted or unsubstituted (3- to 30-membered)heteroarene;
R₅₁ to R₅₅ each independently represent a substituted or unsubstituted (C₆-C₃₀)aryl, a substituted or unsubstituted (C₁-C₃₀)alkyl, a substituted or unsubstituted (C₃-C₃₀)cycloalkyl, or a substituted or unsubstituted (3- to 30-membered)heteroaryl, or may be linked to an adjacent substituent(s) to form a substituted or unsubstituted (C₃-C₃₀)mono- or polycyclic, alicyclic, aromatic ring, or a combination thereof, wherein the carbon atoms of the formed ring may be replaced with at least one heteroatom selected from nitrogen, oxygen, and sulfur;
Ar₅ and Ar₆ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀)aliphatic ring(s) and a (C₆-C₃₀)aromatic ring(s), a substituted or unsubstituted mono- or di(C₁-C₃₀)alkylamino, a substituted or unsubstituted mono- or di(C₂-C₃₀)alkenylamino, a substituted or unsubstituted mono- or di(C₆-C₃₀)arylamino, a substituted or unsubstituted mono- or di(3- to 30-membered)heteroarylamino, a substituted or unsubstituted (C₁-C₃₀)alkyl(C₂-C₃₀)alkenylamino, a substituted or unsubstituted (C₁-C₃₀)alkyl(C₆-C₃₀)arylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, or a substituted or unsubstituted (C₆-C₃₀)aryl(3- to 30-membered)heteroarylamino, or may be linked to an adjacent substituent(s) to form a substituted or unsubstituted (C₃-C₃₀)mono- or polycyclic, alicyclic, aromatic ring, or a combination thereof, wherein the carbon atoms of the formed ring may be replaced with at least one heteroatom selected from nitrogen, oxygen, and sulfur;
L₃ represents a single bond, a substituted or unsubstituted (C₆-C₃₀)arylene, or a substituted or unsubstituted (3- to 30-membered)heteroarylene; and
the heteroaryl(ene) comprises at least one heteroatom selected from B, N, O, S, Si, Se, and P.

According to one embodiment of the present disclosure, the ring C or the ring D each independently represents a substituted or unsubstituted (C₆-C₁₈)arene. According to another embodiment of the present disclosure, the ring C or the ring D each independently represents a substituted or unsubstituted (C₆-C₁₄)arene. For example, the ring C or the ring D each independently may be a benzene ring, a naphthalene ring, a phenanthrene ring, etc.

According to one embodiment of the present disclosure, the ring C and the ring D are each independently selected from the following Formulas 1-5 to 1-9.

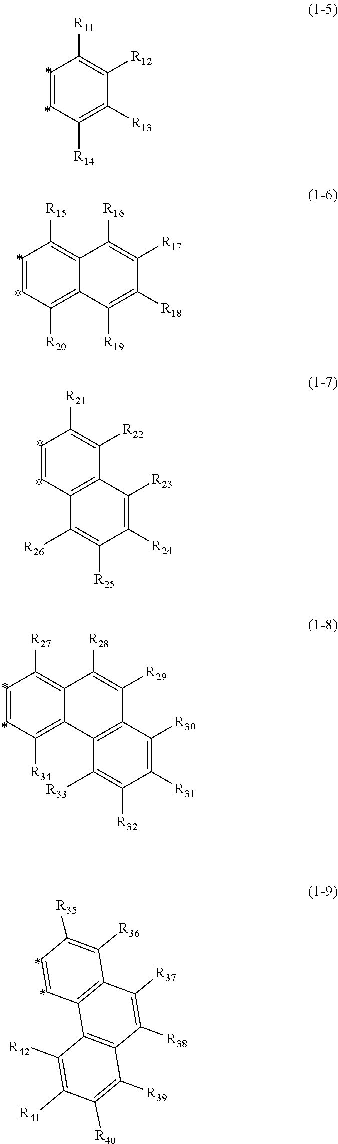

R₁₁ to R₄₂ in Formulas 1-5 to 1-9 each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀)aliphatic ring(s) and a (C₆-C₃₀)aromatic ring(s), a substituted or unsubstituted mono- or di(C₁-C₃₀)alkylamino, a substituted or unsubstituted mono- or di(C₂-C₃₀)alkenylamino, a substituted or unsubstituted mono- or di(C₆-C₃₀)arylamino, a substituted or unsubstituted mono- or di(3- to 30-membered)heteroarylamino, a substituted or unsubstituted (C₁-C₃₀)alkyl(C₂-C₃₀)alkenylamino, a substituted or unsubstituted (C₁-C₃₀)alkyl(C₆-C₃₀)arylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, or a substituted or unsubstituted (C₆-C₃₀)aryl(3- to 30-membered)heteroarylamino, or may be linked to an adjacent substituent(s) to form a substituted or unsubstituted (C₃-C₃₀) mono- or polycyclic, alicyclic, aromatic ring, or a combination thereof, and wherein the carbon atoms of the formed ring may be replaced with at least one heteroatom selected from nitrogen, oxygen, and sulfur.

The compounds represented by Formula 1 according to the present disclosure may be produced by synthetic methods known to one skilled in the art. For example, the compounds of the present disclosure may be synthesized by referring to Korean Patent Application Laid-Open No. 2023-0174704 (Dec. 28, 2023) and Japanese Patent Application Laid-Open No. 1995-287408 (Oct. 31, 1995), but are not limited thereto.

The dopant comprised in the organic electroluminescent device of the present disclosure may be at least one phosphorescent or fluorescent dopant, and is preferably a fluorescent dopant.

The dopant comprised in the organic electroluminescent device of the present disclosure may be a compound represented by the following Formula BD, but is not limited thereto.

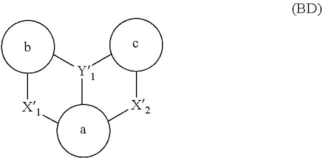

In Formula BD,
Y′₁ represents B or N;
X′₁ and X′₂ each independently represent O, S, NR′, or BR′;
ring a, ring b, and ring c each independently represent a substituted or unsubstituted aromatic hydrocarbon ring having 5 to 50 carbon atoms, a substituted or unsubstituted aromatic heterocyclic ring having 2 to 50 carbon atoms, or a substituted or unsubstituted fused ring of an aliphatic ring having 3 to 30 carbon atoms and an aromatic ring having 3 to 30 carbon atoms;
each R′ independently represents hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or -L′₄-N—(Ar′₄)(Ar′₅); or may be linked to an adjacent substituent(s) to form a ring(s);
L′₄ represents a single bond, a substituted or unsubstituted (C₆-C₃₀)arylene, or a substituted or unsubstituted(3- to 30-membered)heteroarylene; and
Ar′₄ and Ar′₅ each independently represent hydrogen, a substituted or unsubstituted (C₁-C₃₀)alkyl, a substituted or unsubstituted (C₂-C₃₀)alkenyl, a substituted or unsubstituted fused ring of a (C₃-C₃₀)aliphatic ring and a (C₆-C₃₀)aromatic ring, a substituted or unsubstituted (C₆-C₃₀)aryl, or a substituted or unsubstituted (3- to 30-membered)heteroaryl.

According to one embodiment of the present disclosure, Formula BD may be represented by the following Formula BD-1.

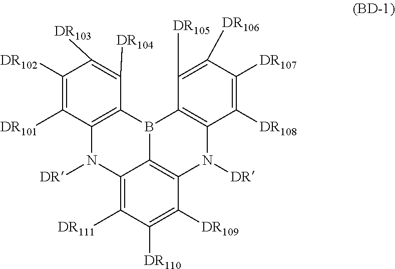

In Formula BD-1,
DR₁₀₁ to DR₁₁₁ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or -L′₄-N—(Ar′₄)(Ar′₅); or may be linked to an adjacent substituent(s) to form a ring(s);
each DR′ independently represents hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or -L′₄-N—(Ar′₄)(Ar′₅); or may be linked to at least one of DR₁₀₁, DR₁₀₈, DR₁₉, and DR₁₁₁ to form a ring(s);
each L′₄ independently represents a single bond, a substituted or unsubstituted (C₆-C₃₀)arylene, or a substituted or unsubstituted(3- to 30-membered)heteroarylene; and
Ar′₄ and Ar′₅ each independently represent hydrogen, a substituted or unsubstituted (C₁-C₃₀)alkyl, a substituted or unsubstituted (C₂-C₃₀)alkenyl, a substituted or unsubstituted fused ring of a (C₃-C₃₀)aliphatic ring and a (C₆-C₃₀)aromatic ring, a substituted or unsubstituted (C₆-C₃₀)aryl, or a substituted or unsubstituted (3- to 30-membered)heteroaryl.

Preferably, DR₁₀₁ to DR₁₁₁ each independently represent hydrogen, deuterium, a substituted or unsubstituted (C₁-C₂₀)alkyl, a substituted or unsubstituted (C₆-C₂₅)aryl, a substituted or unsubstituted (5- to 25-membered)heteroaryl, or -L′₄-N—(Ar′₄)(Ar′₅); or may be linked to an adjacent substituent(s) to form a ring(s).

More preferably, DR₁₀₁ to DR₁₁₁ each independently represent hydrogen, deuterium, an unsubstituted (C₁-C₁₀)alkyl; a (C₆-C₁₈)aryl unsubstituted or substituted with at least one of (C₁-C₁₀)alkyl, (13- to 18-membered)heteroaryl, and di(C₆-C₁₈)arylamino; a (5- to 18-membered)heteroaryl unsubstituted or substituted with at least one of (C₁-C₁₀)alkyl; or -L′₄-N—(Ar′₄)(Ar′₅); or may be linked to an adjacent substituent(s) to form a ring(s). For example, DR₁₀₁ to DR₁₁₁ each independently may be hydrogen, a methyl, a tert-butyl, a substituted or unsubstituted phenyl, a biphenyl, a terphenyl, a triphenylenyl, a carbazolyl, a phenoxazinyl, a phenothiazinyl, a dimethylacridinyl, a dimethylxanthenyl, a diphenylamino unsubstituted or substituted with at least one of a methyl and a diphenylamino, a phenylnaphthylamino, a dibiphenylamino, a phenylamino substituted with a phenylcarbazolyl or a dibenzofuranyl, a (17- to 21-membered)heteroaryl substituted with at least one of a methyl and a phenyl; or may be linked to an adjacent substituent(s) to form a benzene ring, an indole ring substituted with at least one of a phenyl and a diphenylamino, a benzofuran ring, a benzothiophene ring, or a 19-membered hetero ring substituted with at least one of a methyl. The substituted phenyl may be substituted with one or more of a methyl, a carbazolyl, a dibenzofuranyl, a diphenylamino, a phenoxazinyl, a phenothiazinyl, and a dimethylacridinyl.

According to another embodiment of the present disclosure, Formula BD may be represented by the following Formula BD-2 or BD-3.

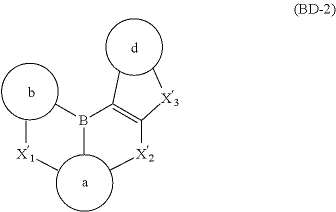

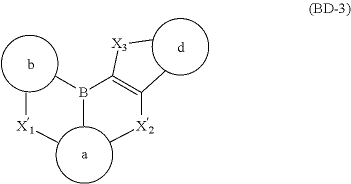

In Formulas BD-2 and BD-3,
X′₁ and X′₂ each independently represent O, S, NR′, or BR′;
X′₃ represents O or S;
ring a, ring b, and ring d each independently represent a substituted or unsubstituted aromatic hydrocarbon ring having 5 to 50 carbon atoms, a substituted or unsubstituted aromatic heterocyclic ring having 2 to 50 carbon atoms, or a substituted or unsubstituted fused ring of an aliphatic ring having 3 to 30 carbon atoms and an aromatic ring having 3 to 30 carbon atoms; and
R′, L′₄, Ar′₄, and Ar′₅ are as defined in Formula BD.

According to one embodiment of the present disclosure, Formula BD may be particularly exemplified by the following compounds, but is not limited thereto.

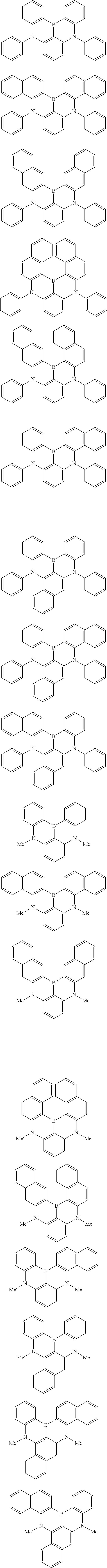

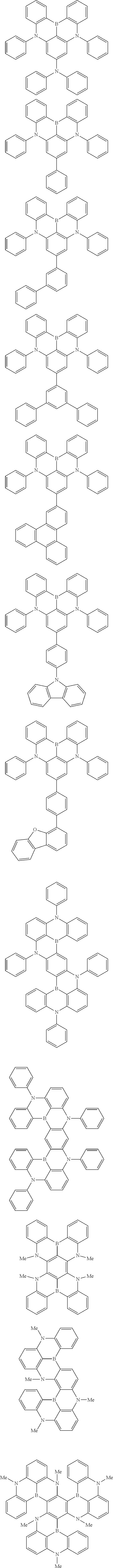

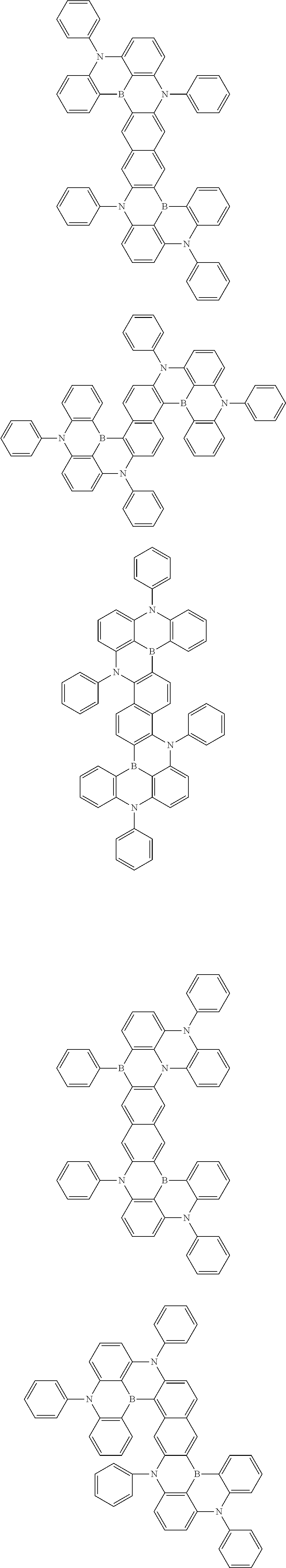

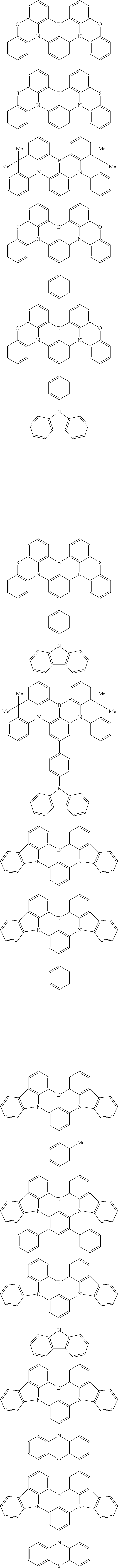

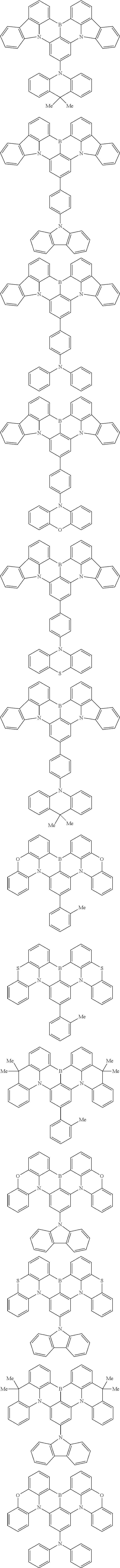

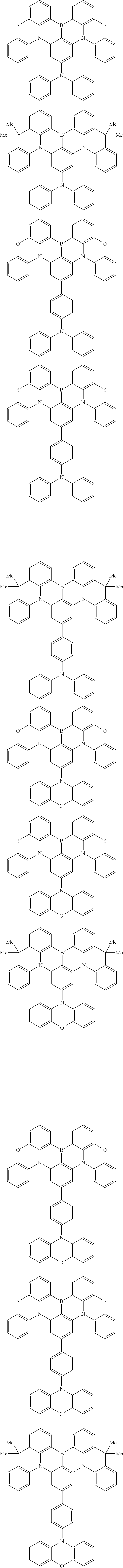

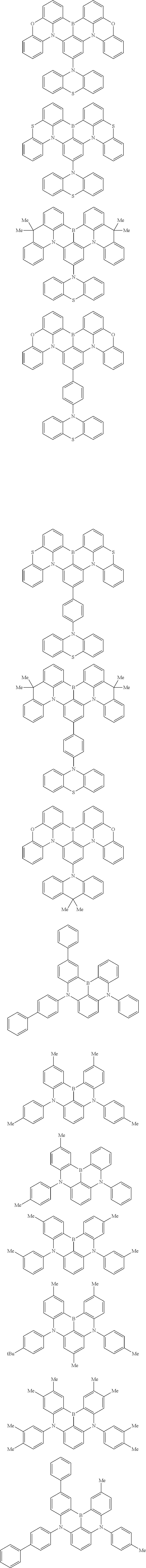

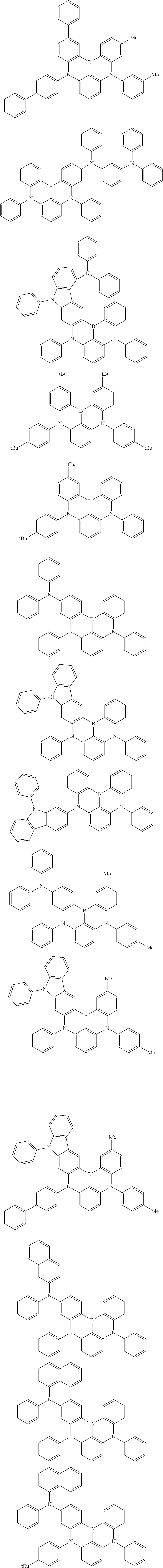

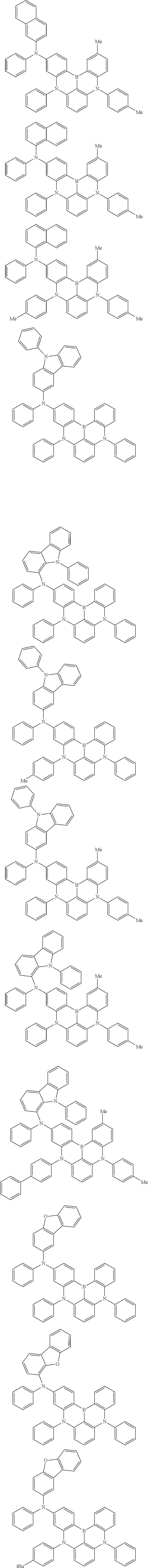

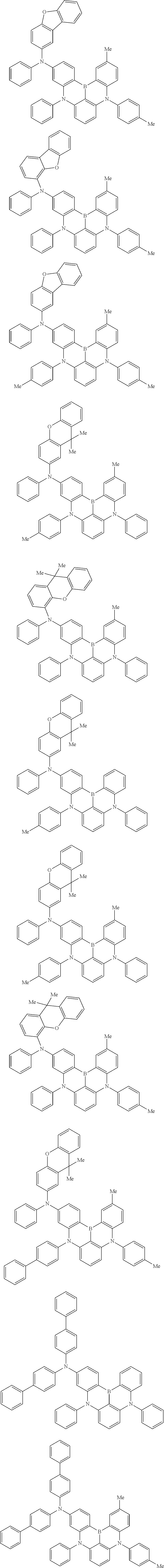

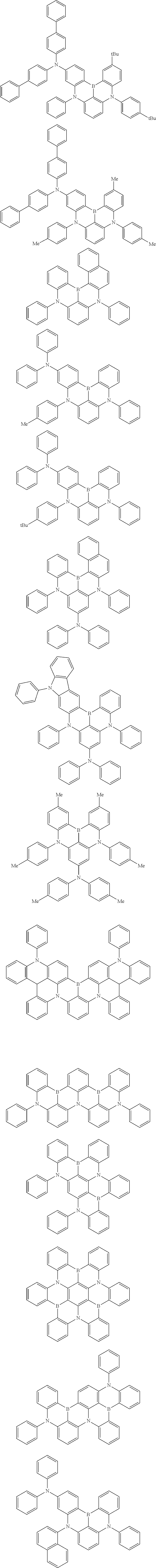

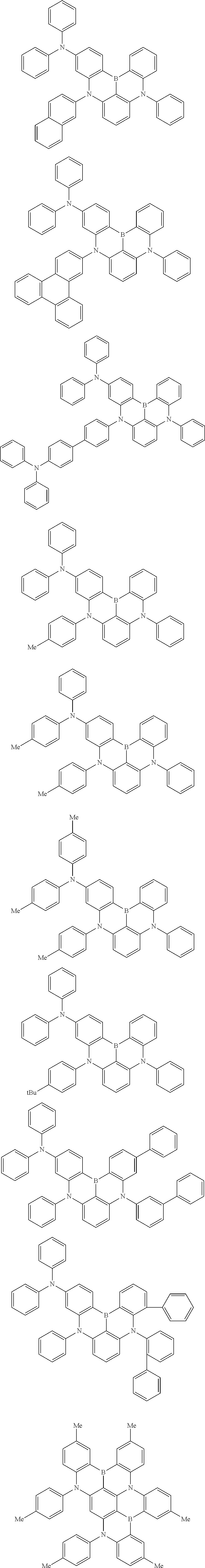

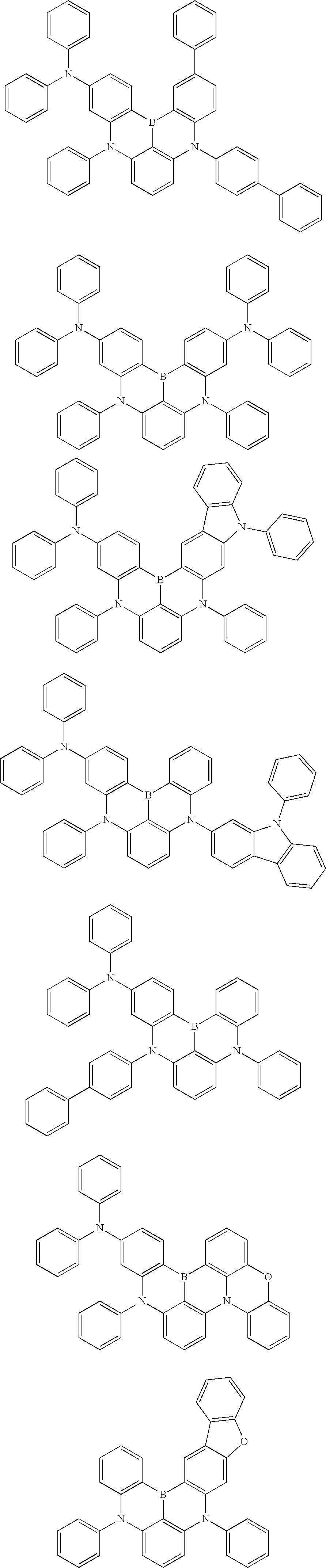

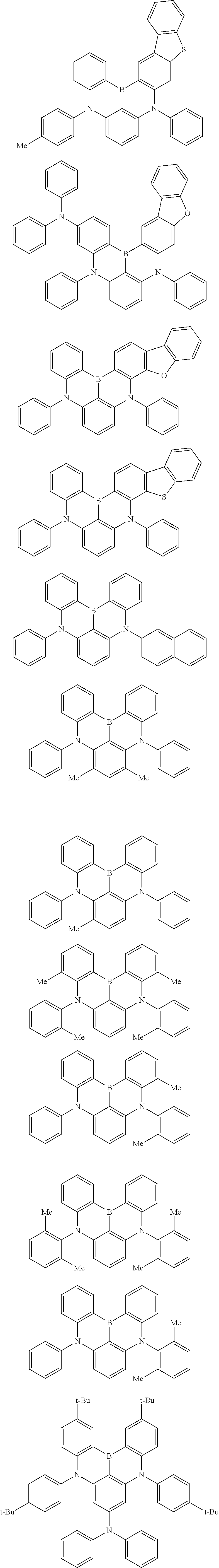

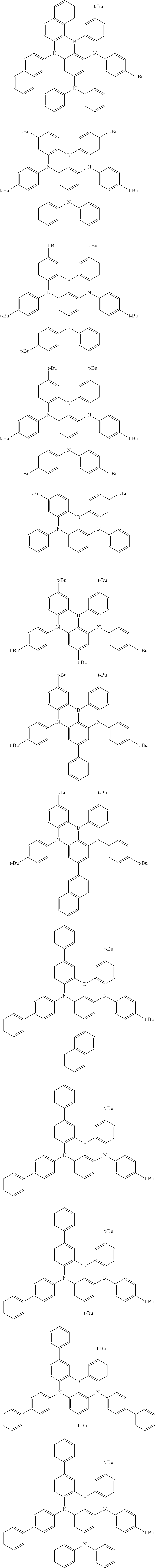

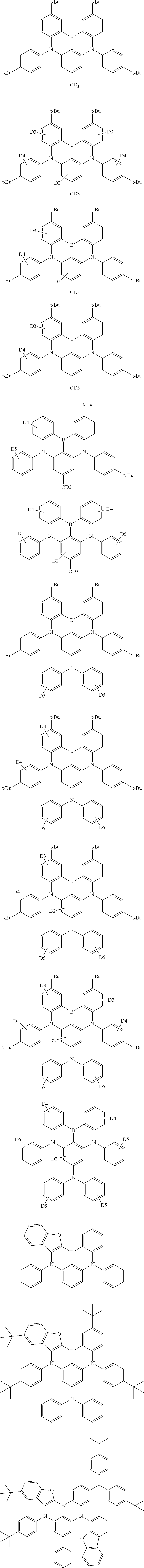

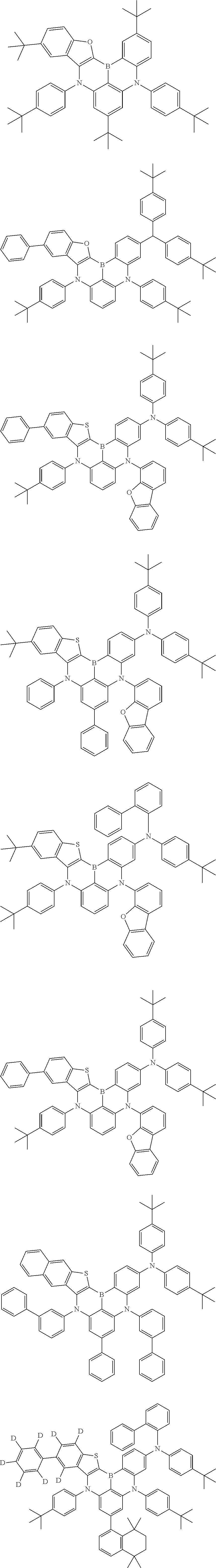

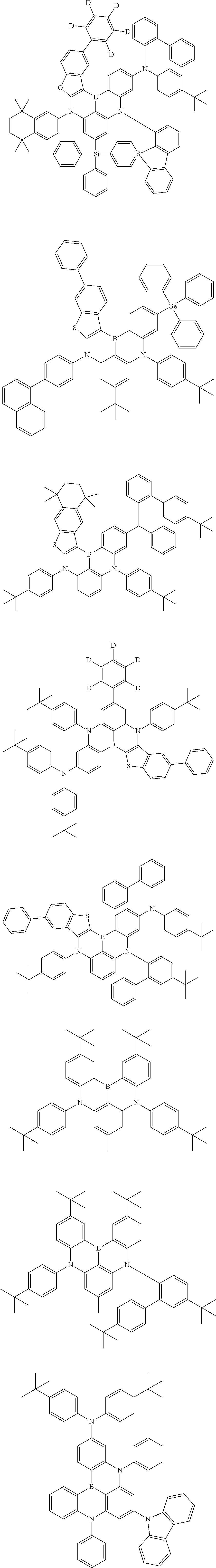

In the above compounds, D2 to D5 mean that 2 to 5 hydrogens are replaced by deuterium, respectively.

The dopant comprised in the organic electroluminescent device of the present disclosure may be a compound represented by the following Formula 101, but is not limited thereto.

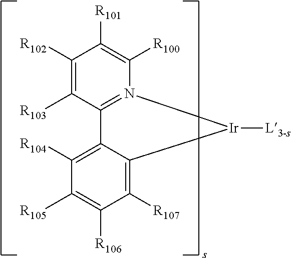

In Formula 101,
L′ is any one selected from the following Structures 1 to 3:

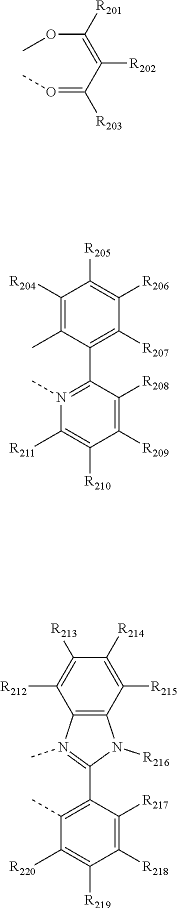

R₁₀₀ to R₁₀₃ each independently represent hydrogen, deuterium, a halogen, a (C₁-C₃₀)alkyl unsubstituted or substituted with deuterium and/or a halogen(s), a substituted or unsubstituted (C₃-C₃₀)cycloalkyl, a substituted or unsubstituted (C₆-C₃₀)aryl, a cyano, a substituted or unsubstituted (3- to 30-membered)heteroaryl, or a substituted or unsubstituted (C₁-C₃₀)alkoxy; or may be linked to an adjacent substituent(s) to form a ring(s), for example, to form a ring(s) with a pyridine, e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;
R₁₀₄ to R₁₀₇ each independently represent, hydrogen, deuterium, halogen, a (C₁-C₃₀)alkyl unsubstituted or substituted with deuterium and/or a halogen(s), a substituted or unsubstituted (C₃-C₃₀)cycloalkyl, a substituted or unsubstituted (C₆-C₃₀)aryl, a substituted or unsubstituted (3- to 30-membered)heteroaryl, cyano, or a substituted or unsubstituted (C₁-C₃₀)alkoxy; or may be linked to an adjacent substituent(s) to form a substituted or unsubstituted ring(s), for example, to form a ring(s) with a benzene, e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;
R₂₀₁ to R₂₂₀ each independently represent, hydrogen, deuterium, halogen, a (C₁-C₃₀)alkyl unsubstituted or substituted with deuterium and/or a halogen(s), a substituted or unsubstituted (C₃-C₃₀)cycloalkyl, or a substituted or unsubstituted (C₆-C₃₀)aryl; or may be linked to an adjacent substituent(s) to form a ring(s); and
s represents an integer of 1 to 3.

Specifically, the specific examples of the dopant compound include the following, but are not limited thereto.

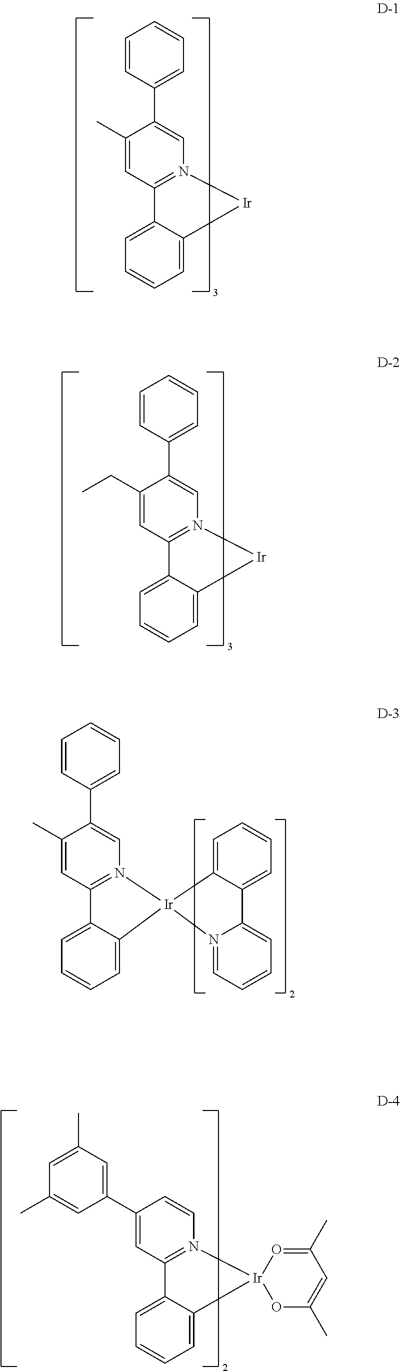

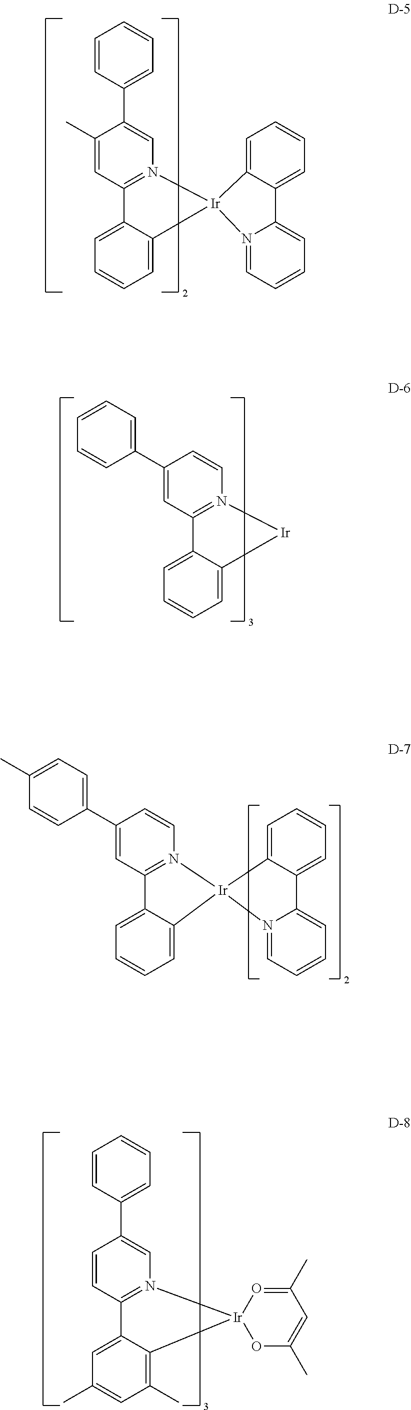

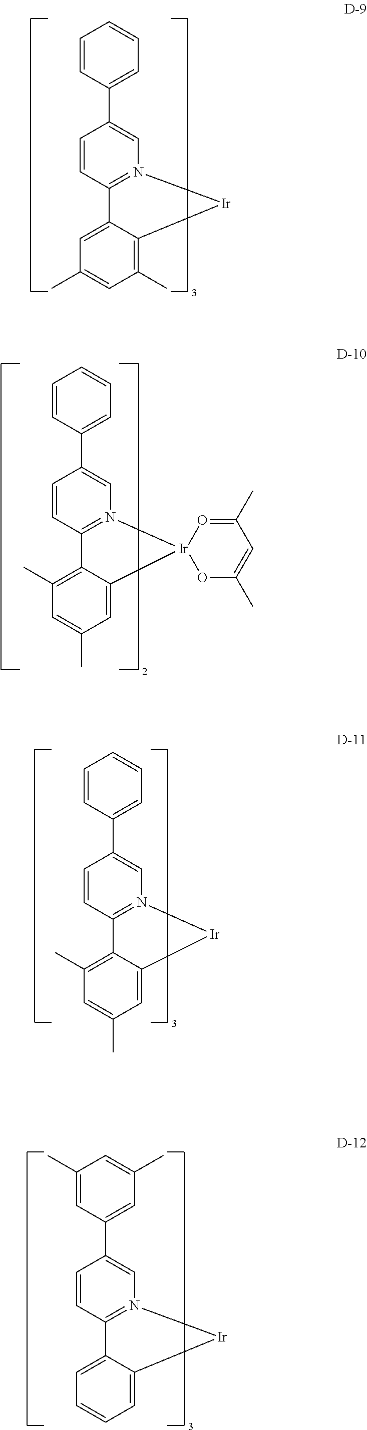

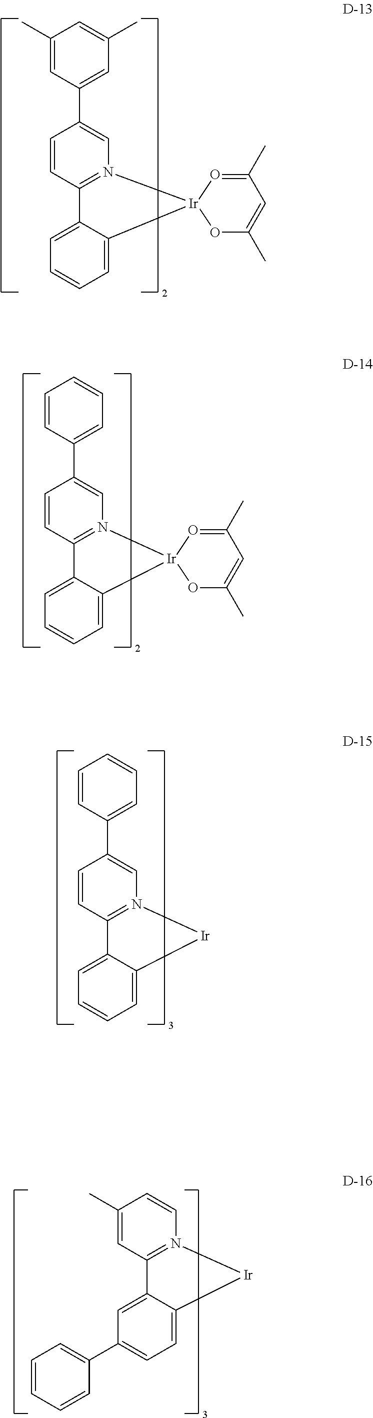

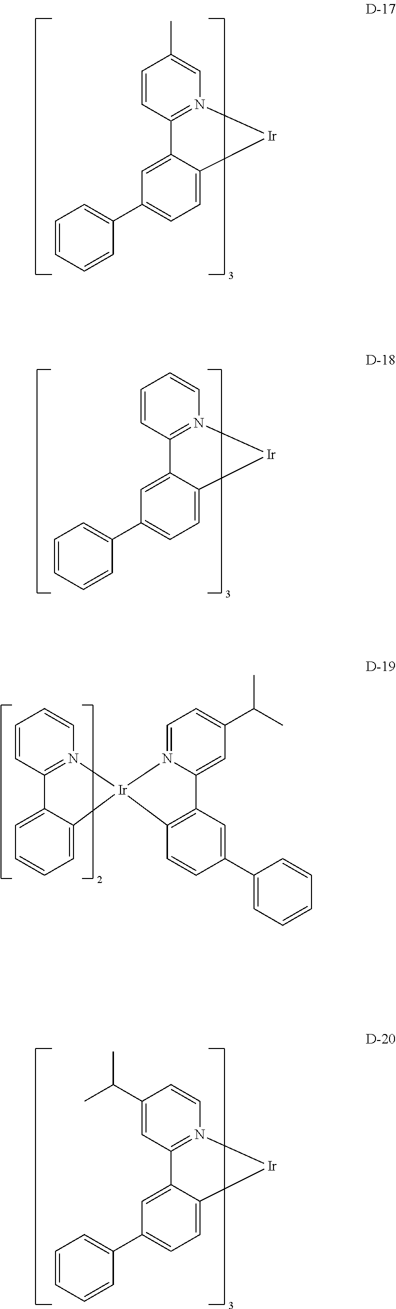

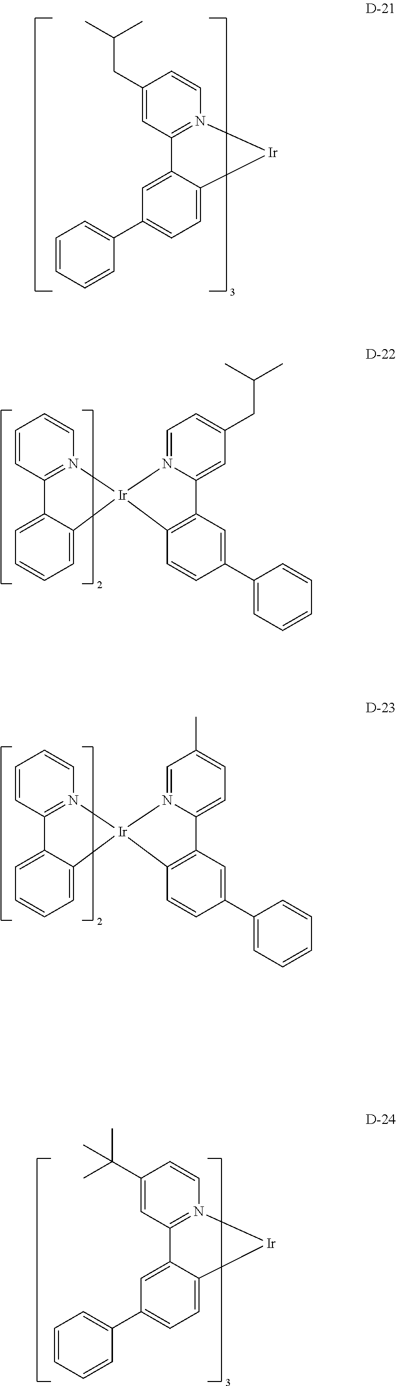

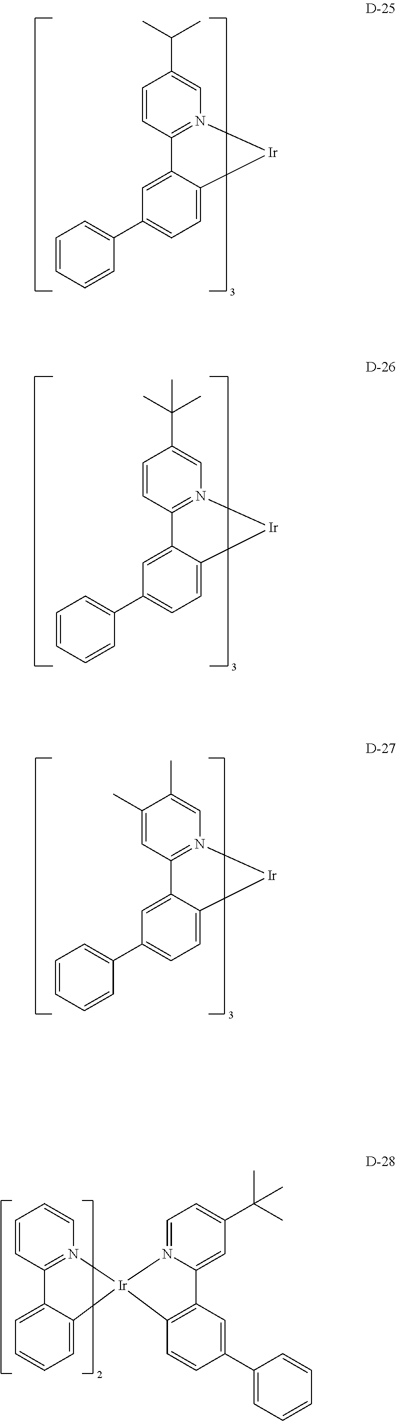

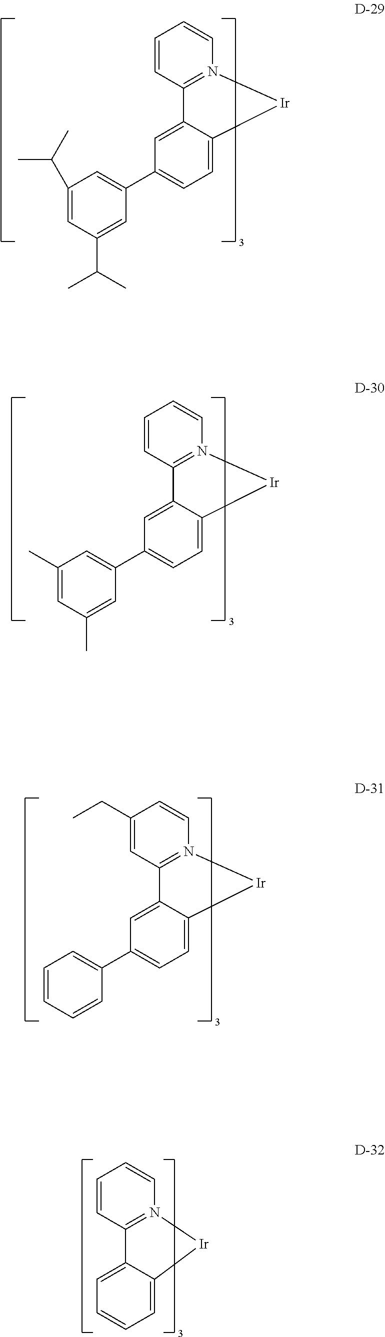

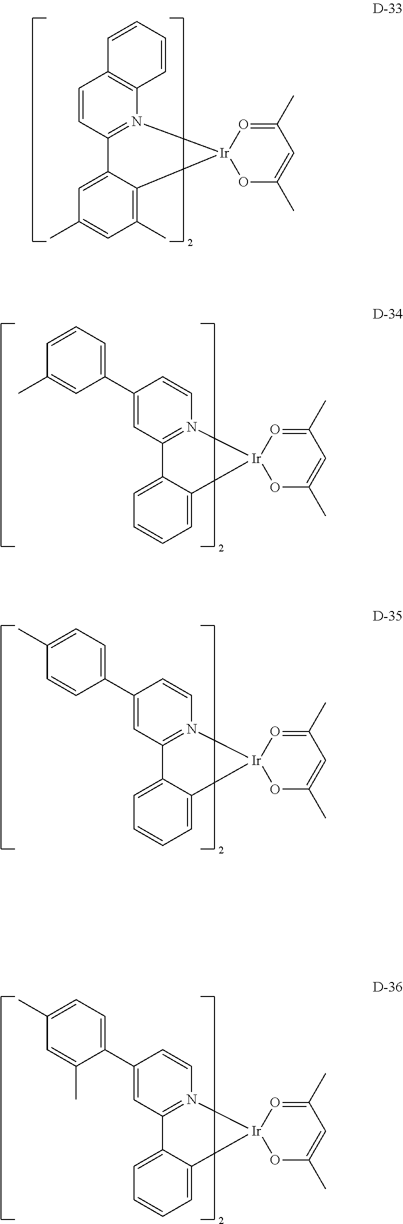

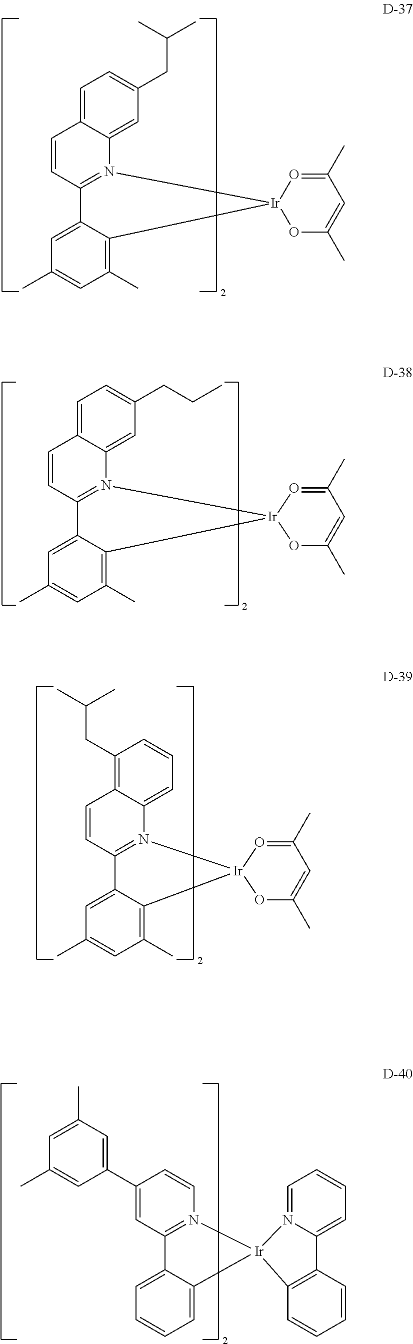

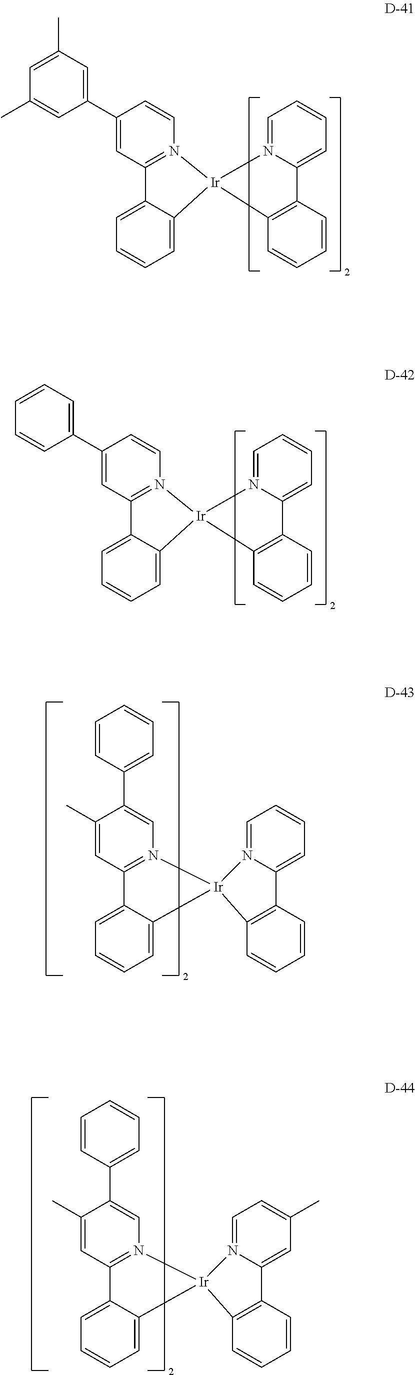

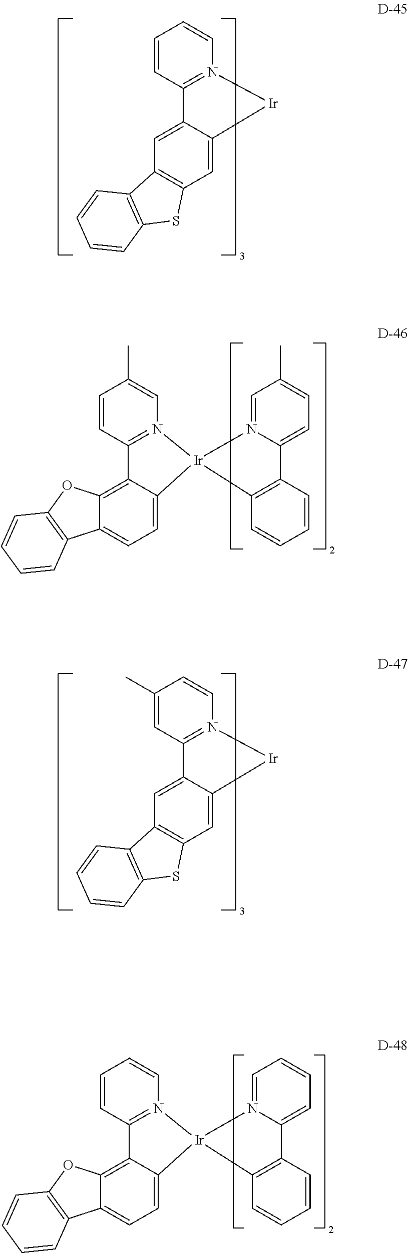

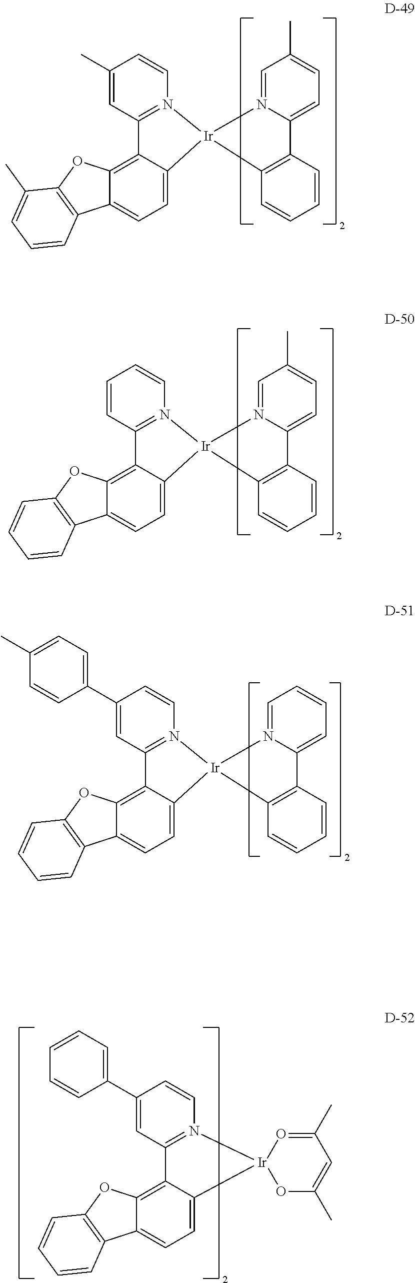

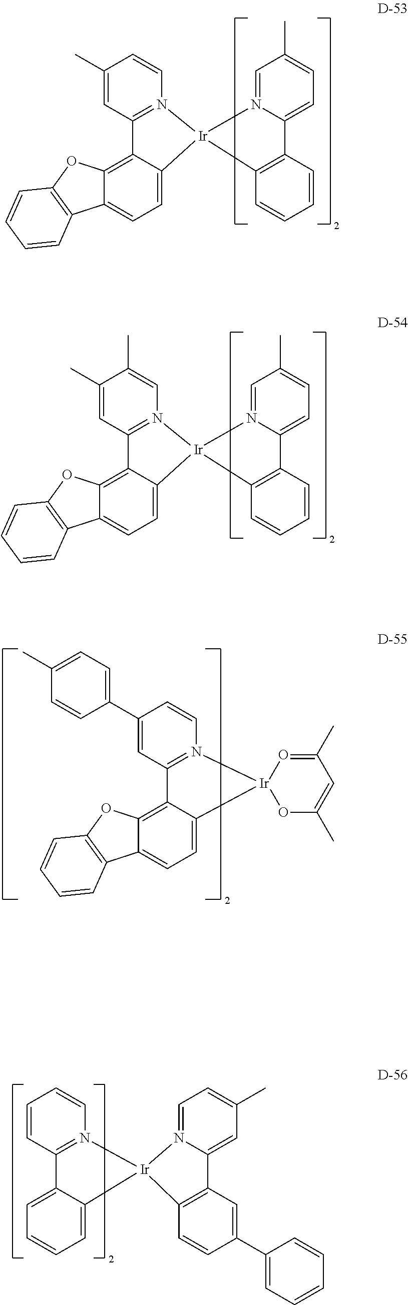

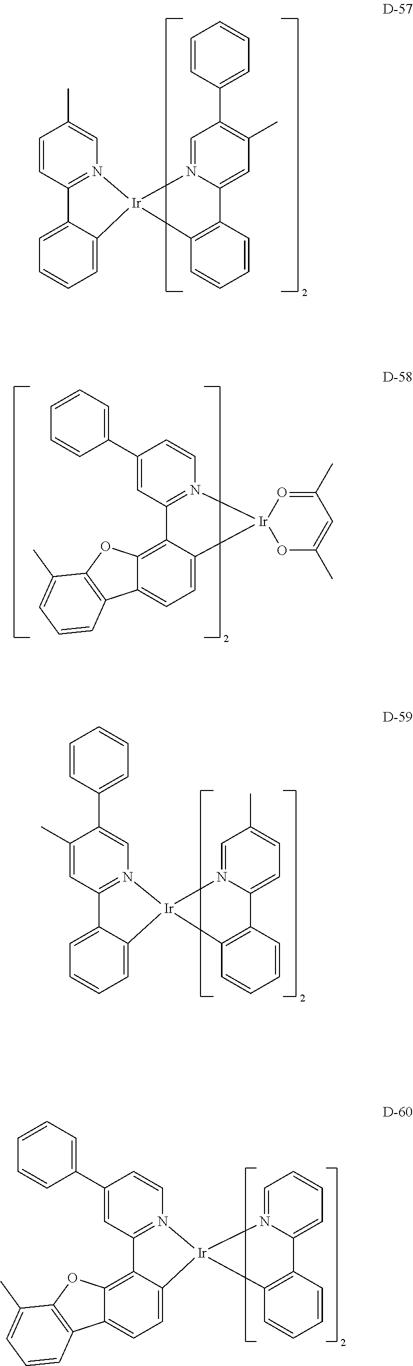

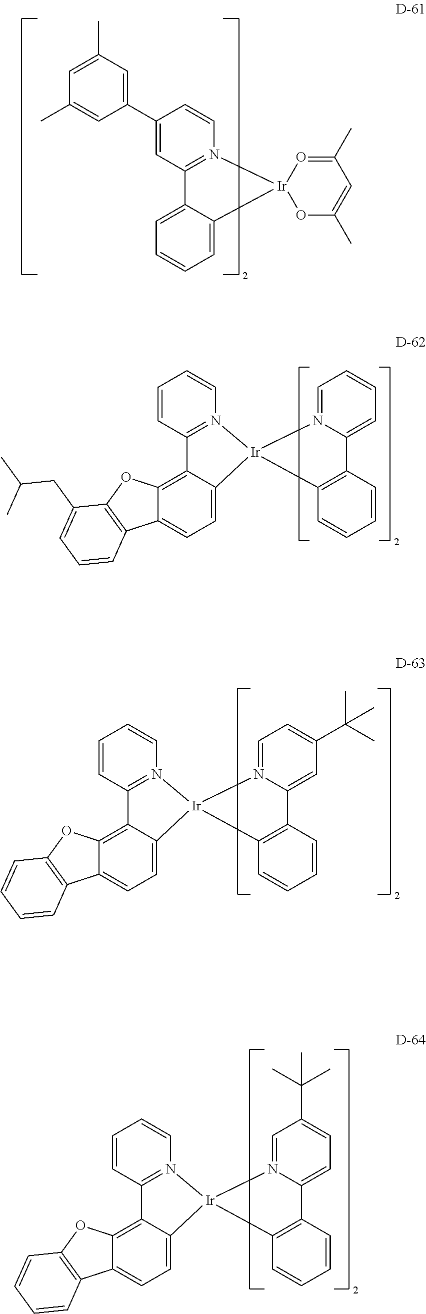

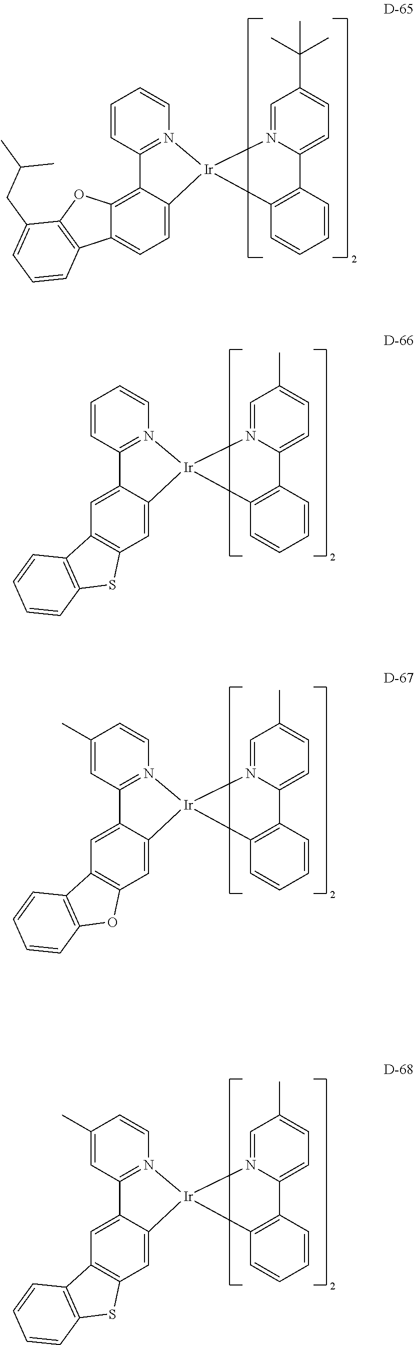

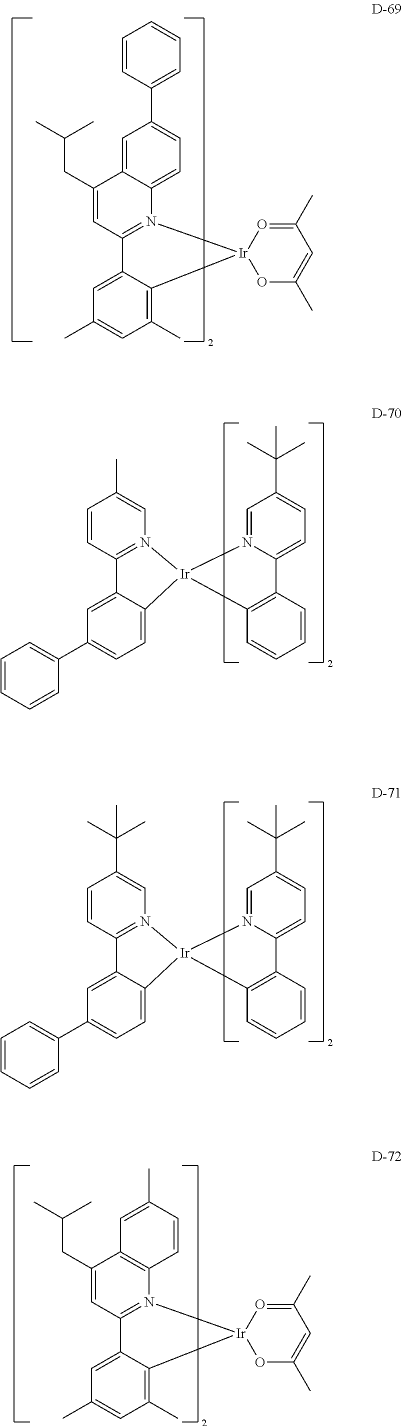

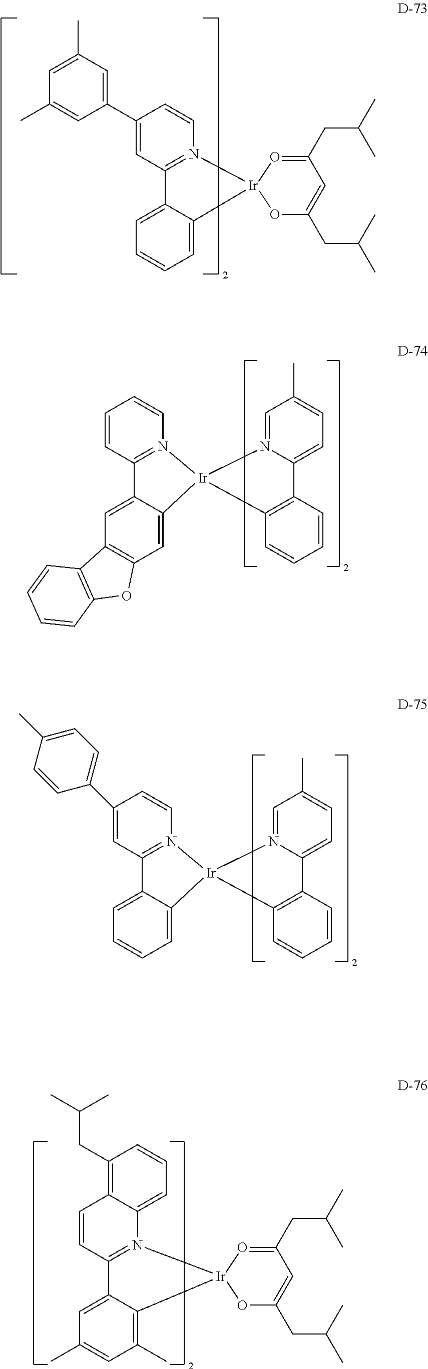

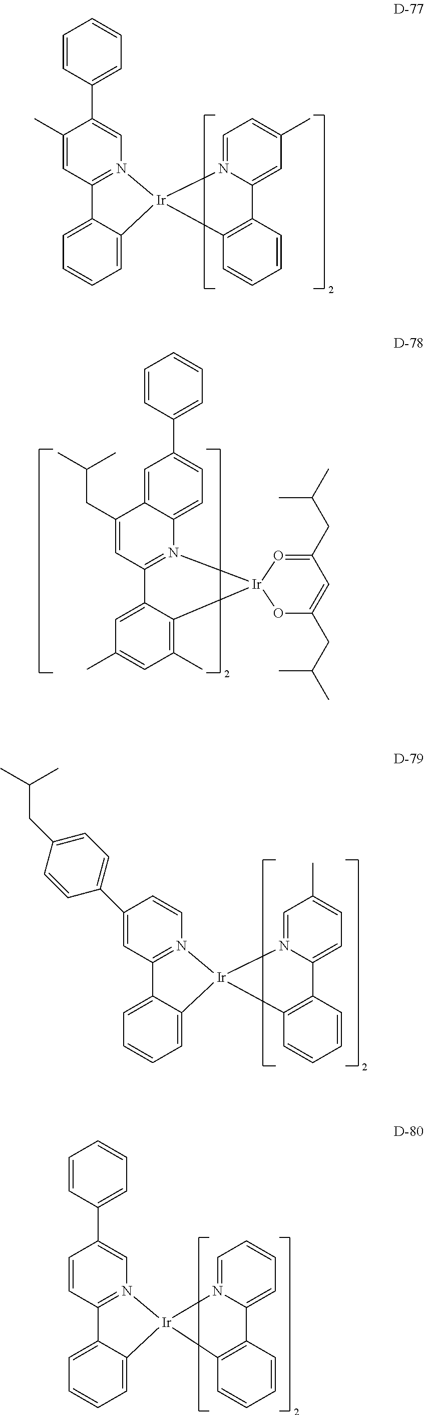

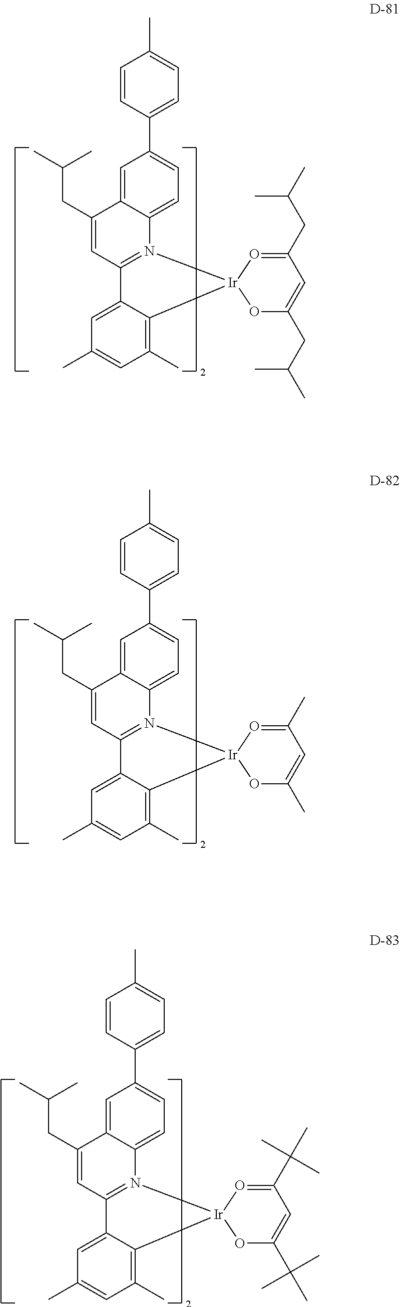

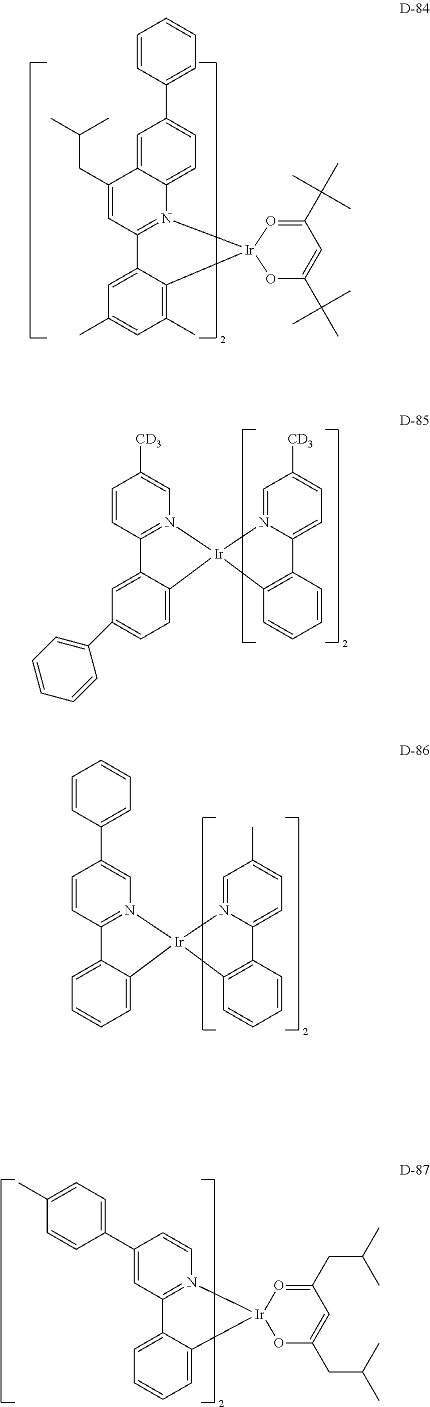

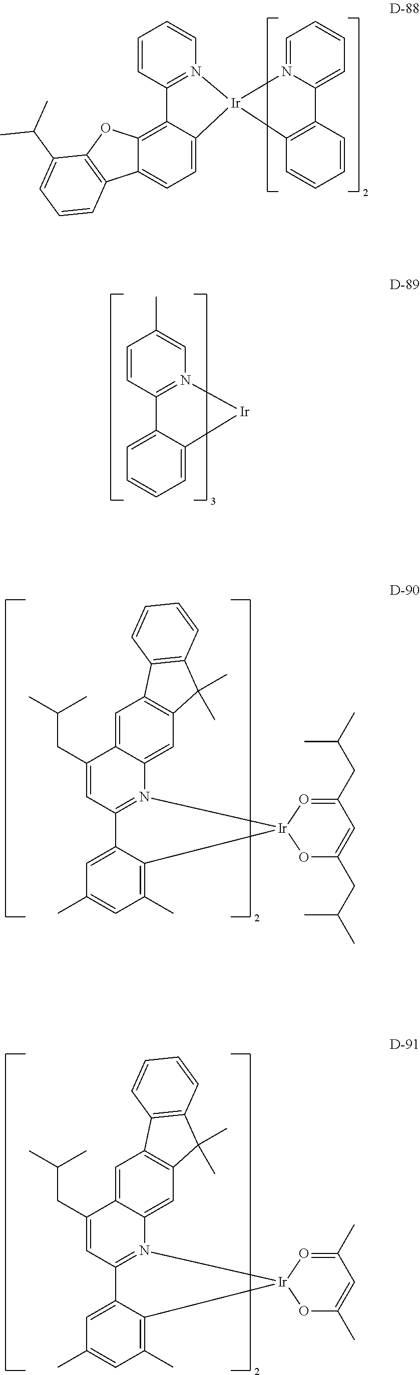

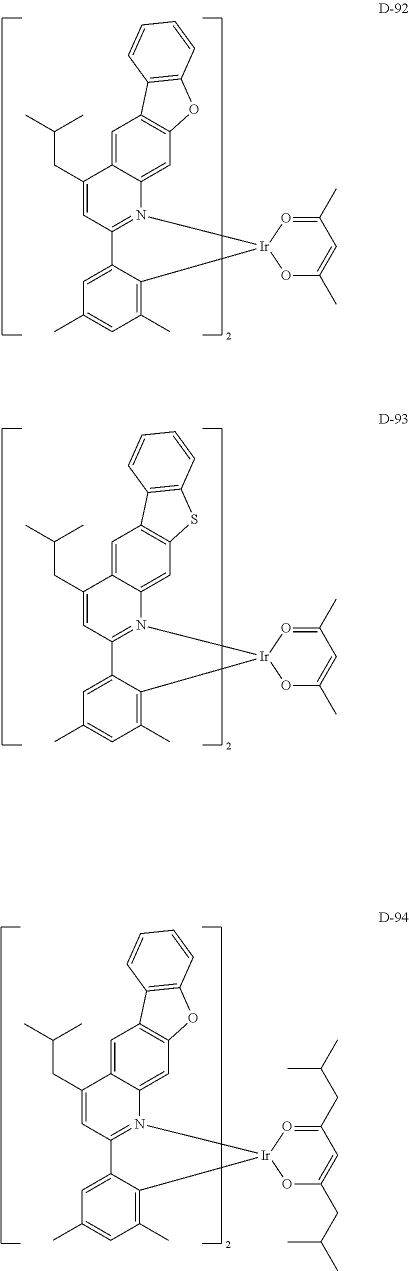

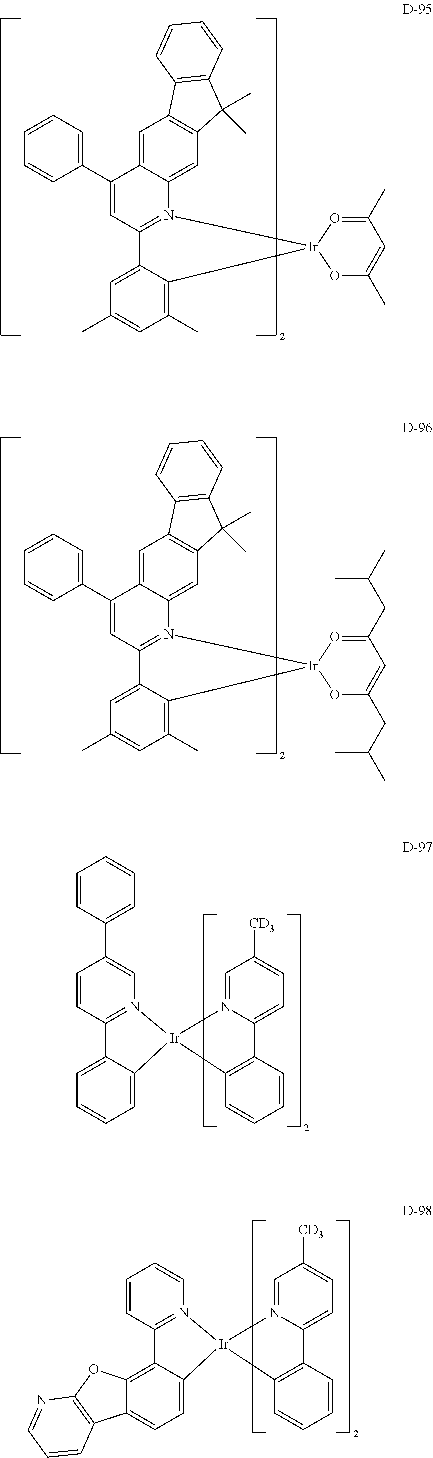

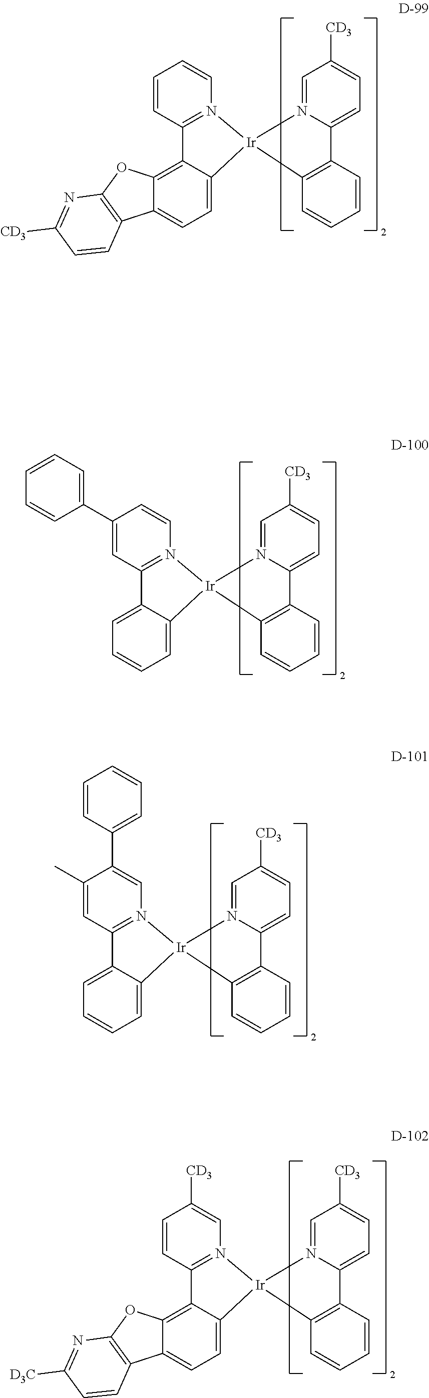

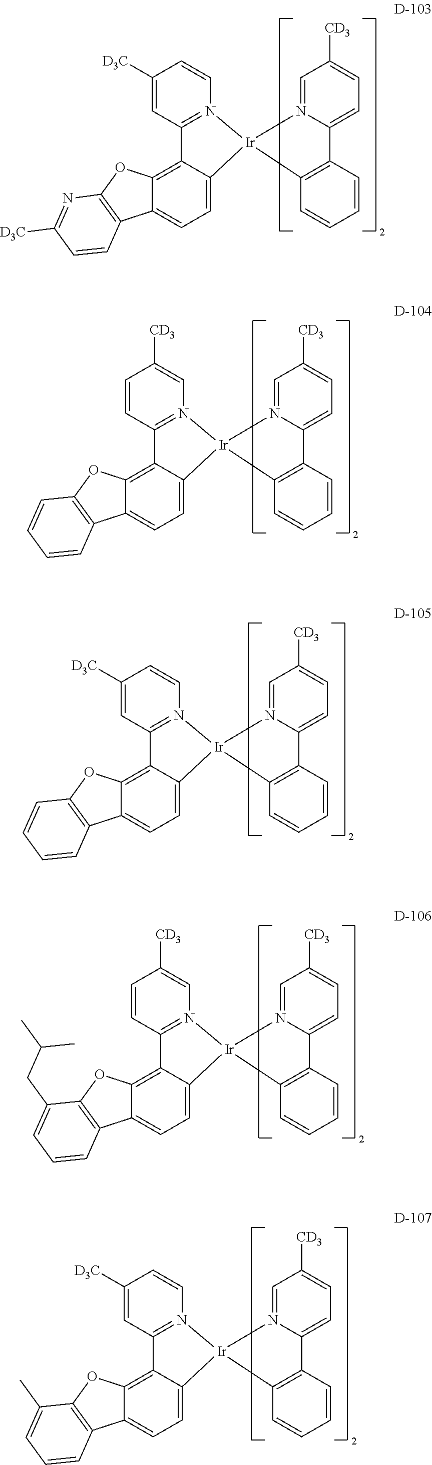

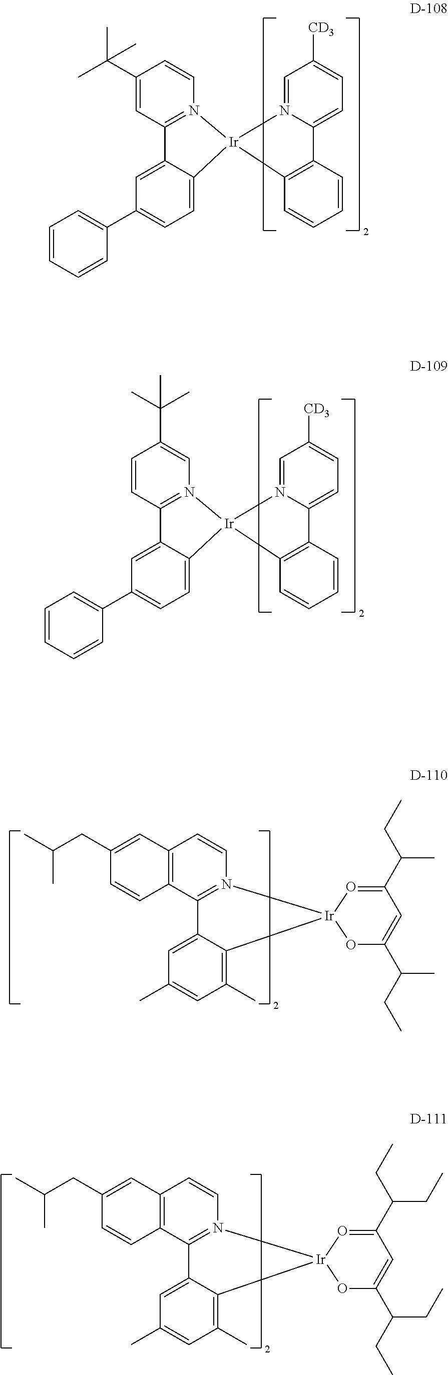

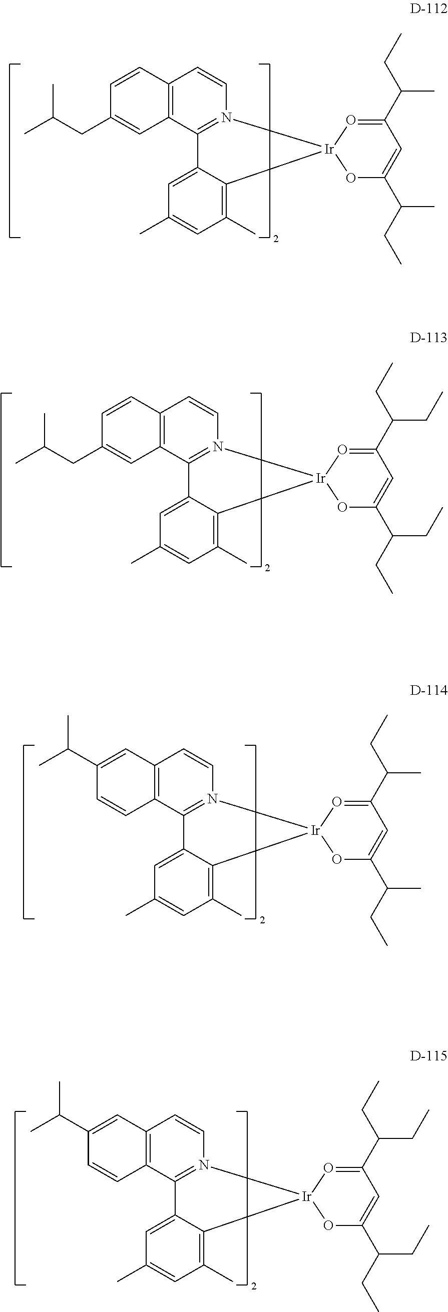

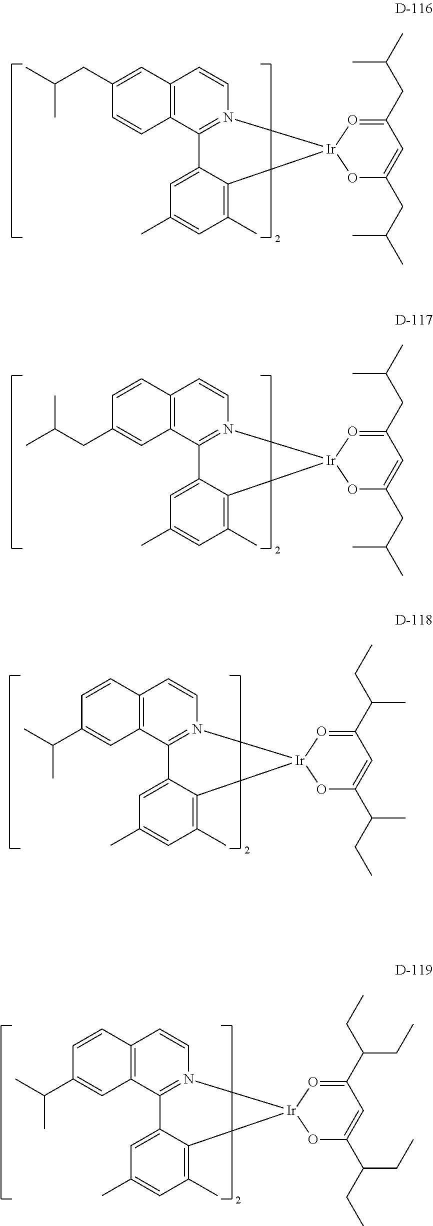

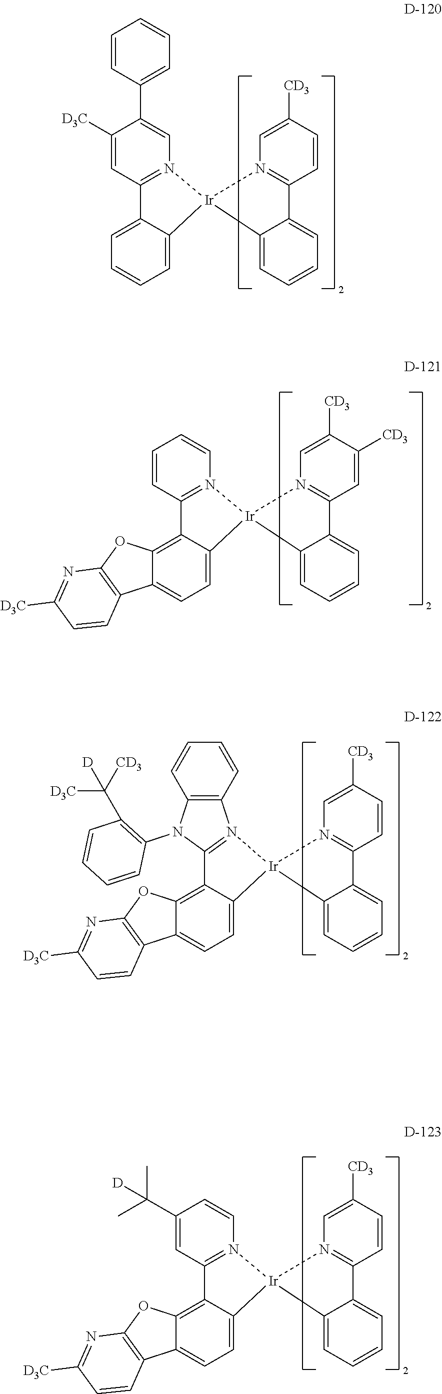

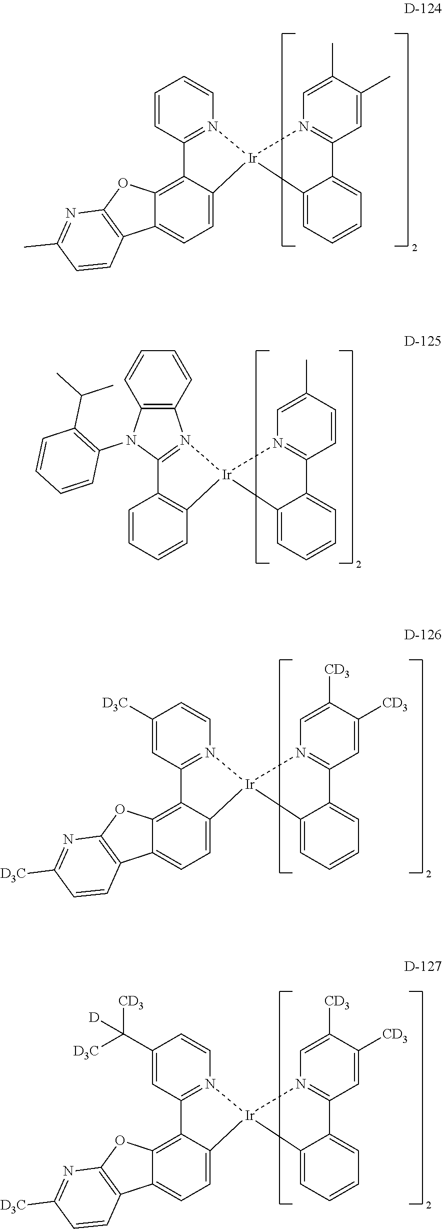

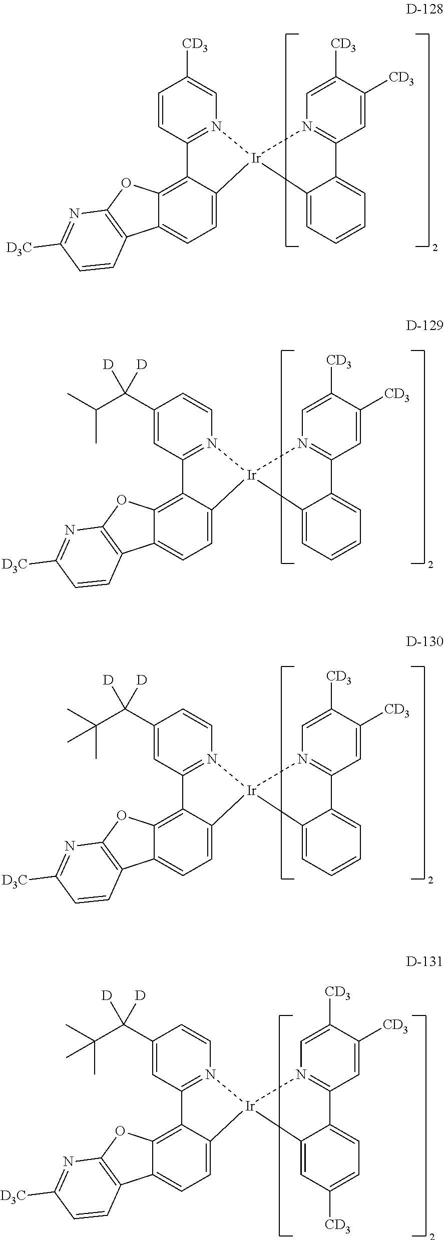

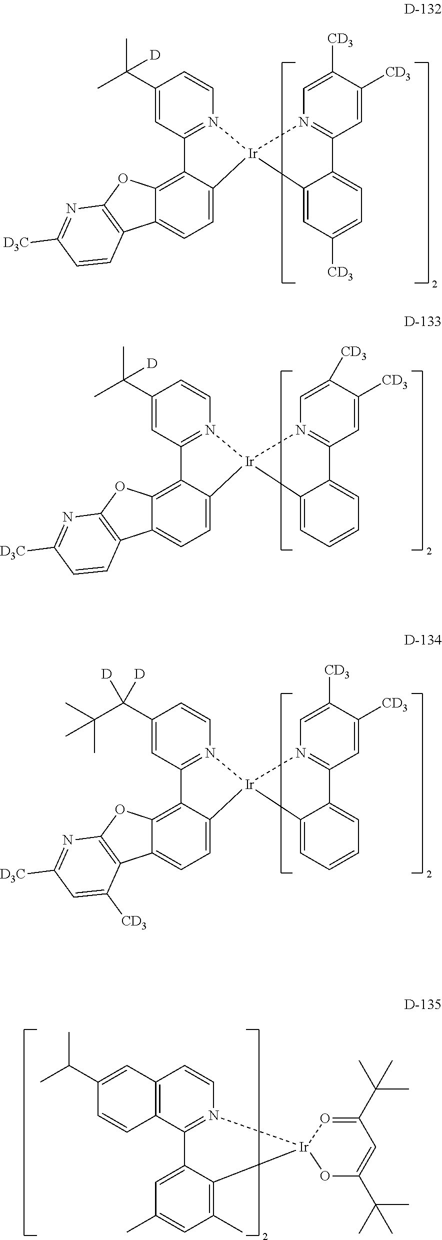

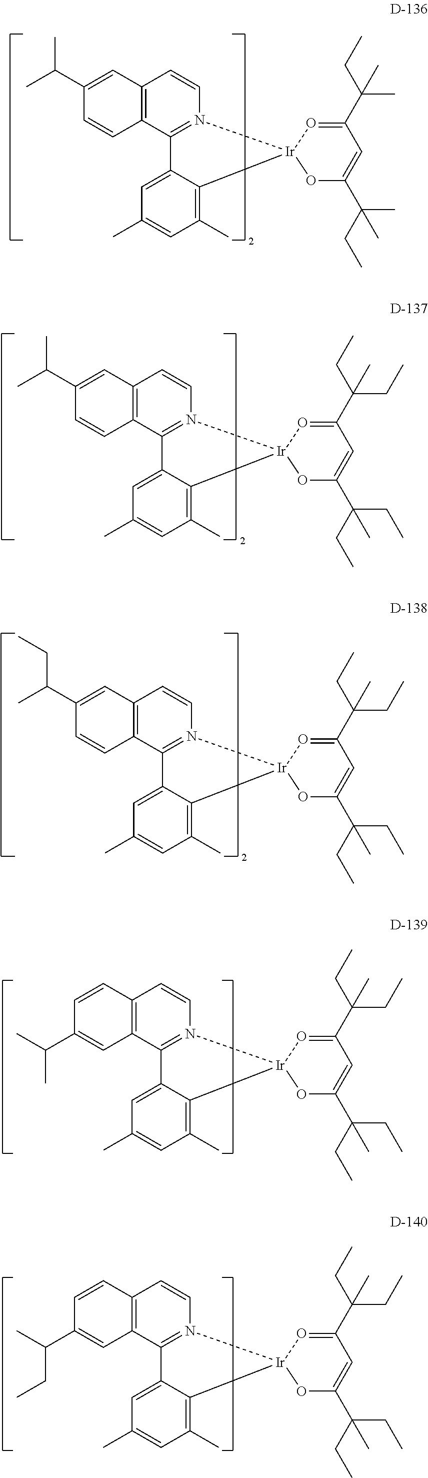

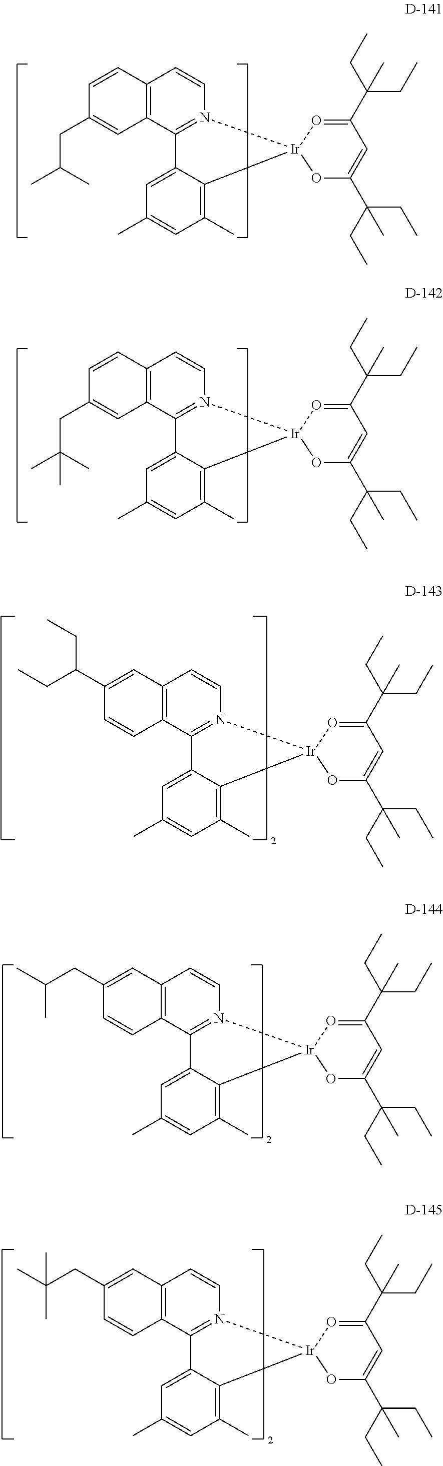

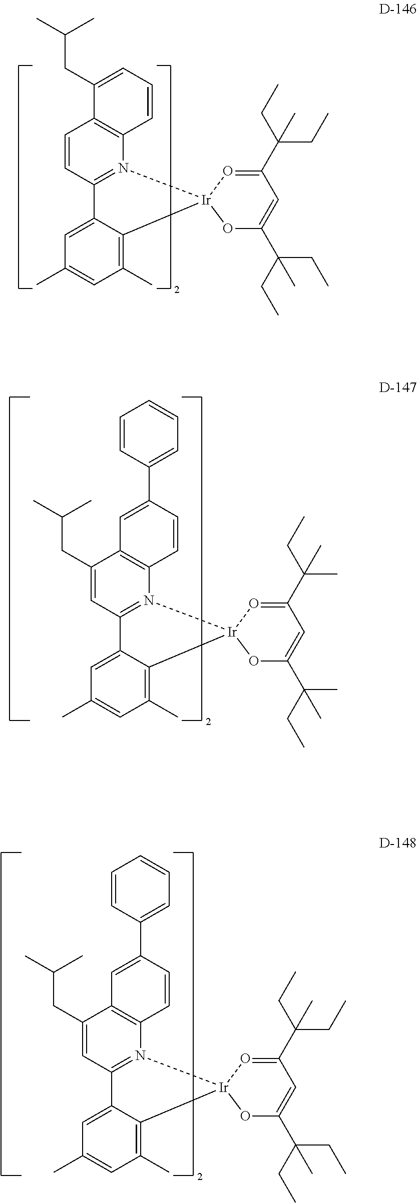

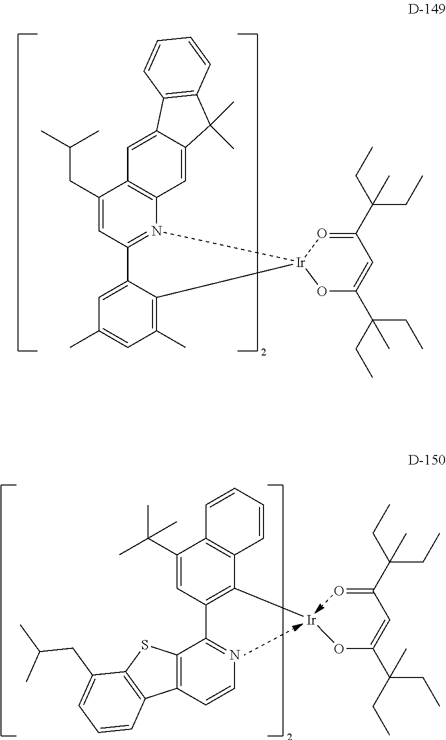

The organic electroluminescent device according to the present disclosure comprises a first electrode; a second electrode facing the first electrode; and an organic layer between the first electrode and the second electrode, wherein the organic layer comprises a hole transport region, a light-emitting layer, and an electron transport region, wherein the hole transport region comprises a hole injection layer and a plurality of hole transport layers, wherein the hole injection layer comprises a compound represented by Formula 1, wherein each of the plurality of hole transport layers comprises at least one compound, and wherein at least two of the plurality of hole transport layers are different from each other. At least one of the plurality of hole transport layers comprises a compound represented by Formula 2.

One of the first and second electrodes may be an anode, and the other may be a cathode. The first electrode and the second electrode may each be formed with a transmissive conductive material, a transflective conductive material, or a reflective conductive material. The organic electroluminescent device may be a top emission type, a bottom emission type, or a both-sides emission type depending on the type of the material forming the first electrode and the second electrode.

According to one embodiment of the present disclosure, the hole injection layer further comprises at least one p-dopant. According to another embodiment of the present disclosure, the hole injection layer does not comprise a p-dopant.

According to one embodiment of the present disclosure, the plurality of hole transport layers comprises a first hole transport layer, a second hole transport layer, and a third hole transport layer. According to another embodiment of the present disclosure, the plurality of hole transport layers comprises at least three layers. The plurality of hole transport layers comprises a first hole transport layer, a second hole transport layer, and a third hole transport layer, and a compound comprised in the first hole transport layer, a compound comprised in the second hole transport layer, and a compound comprised in the third hole transport layer may be each different from one another. According to one embodiment of the present disclosure, the plurality of hole transport layers may comprise at least one of a hole transport layer, a hole auxiliary layer, and an electron-blocking layer. For example, the plurality of hole transport layers may comprise one or more hole transport layers and two or more hole auxiliary layers. In addition, for example, the plurality of hole transport layers may comprise one or more hole transport layers, one or more hole auxiliary layers, and one or more electron-blocking layers.

According to one embodiment of the present disclosure, the light-emitting layer comprises a plurality of light-emitting layers comprising at least two layers, and a first light-emitting layer and a second light-emitting layer are comprised adjacently. According to another embodiment of the present disclosure, the light-emitting layer comprises a plurality of light-emitting layers comprising at least three layers, and a first light-emitting layer, a second light-emitting layer, and a third light-emitting layer are comprised sequentially adjacent to one another.

According to one embodiment of the present disclosure, the organic layer further comprises a first stack and a second stack, wherein a charge generation region is comprised between the first stack and the second stack, and wherein the charge generation region comprises an N-type charge generation layer, and a P-type charge-generation layer. The N-type charge generation layer comprises a compound having a structure comprising at least one of a phenanthroline and a terpyridine, and the N-type charge generation layer may be doped with Li or Yb.

According to one embodiment of the present disclosure, the organic layer may further comprise at least one layer selected from a hole auxiliary layer, a light-emitting auxiliary layer, an electron transport layer, an electron buffer layer, an electron injection layer, an interlayer, a hole-blocking layer, and an electron-blocking layer. Each of the layers may be further configured with multiple layers. In addition, the electron injection layer may be further doped with an n-type dopant.

The organic layer may additionally comprise at least one compound selected from the group consisting of an arylamine-based compound, and a styrylarylamine-based compound. In addition, the organic layer may further comprise at least one metal selected from the group consisting of metals of Group 1, metals of Group 2, transition metals of the 4th period, transition metals of the 5th period, lanthanides, and organic metals of the d-transition elements of the Periodic Table, or at least one complex compound comprising the metal.

In addition, the organic electroluminescent device of the present disclosure may emit white light by further comprising at least one light-emitting layer comprising blue, red, or green light-emitting compounds known in the art. Furthermore, if necessary, the device may further comprise a yellow or orange light-emitting layer. The white organic light-emitting device has been suggested to have various structures such as a side-by-side arrangement method, a stacking arrangement method, or a color conversion material (CCM) method, etc., according to the arrangement of R (red), G (green), YG (yellowish green), or B (blue) light-emitting units.

In the organic electroluminescent device of the present disclosure, it is preferable to dispose at least one layer selected from a chalcogenide layer, a metal halide layer, and a metal oxide layer (hereinafter referred to as a “surface layer”) on at least one inner surface of a pair of electrodes. Specifically, a chalcogenide (including an oxide) layer of silicon and aluminum is preferably placed on an anode surface of an electroluminescent medium layer side, and a metal halide layer or a metal oxide layer is preferably placed on a cathode surface of an electroluminescent medium layer side. Driving stabilization of the organic electroluminescent device can be obtained by the surface layer. Preferred examples of the chalcogenide include SiO_{X} (1≤X≤2), AlO_{X} (1≤X≤1.5), SiON, SiAlON, etc., preferred examples of the metal halide include LiF, MgF₂, CaF₂, a rare earth metal fluoride, etc., and preferred examples of the metal oxide include Cs₂O, Li₂O, MgO, SrO, BaO, CaO, etc.

A hole injection layer, a hole transport layer, an electron-blocking layer, or a combination thereof may be used between an anode and a light-emitting layer. The hole injection layer may be multi-layered in order to lower the hole injection barrier (or hole injection voltage) from the anode to the hole transport layer or the electron-blocking layer, wherein two compounds may be simultaneously used in each of the multi-layers. The hole transport layer or the electron-blocking layer may also be multi-layers.

An electron buffer layer, a hole-blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole-blocking layer or the electron transport layer may also be multi-layers, and each layer may use a plurality of compounds.

A light-emitting auxiliary layer may be a layer placed between an anode and a light-emitting layer, or between a cathode and a light-emitting layer. When placed between the anode and the light-emitting layer, the light-emitting auxiliary layer may be used to facilitate hole injection and/or hole transport or to block the overflow of electrons. When placed between the cathode and the light-emitting layer, the light-emitting auxiliary layer may be used to facilitate electron injection and/or electron transport or to block the overflow of holes. In addition, the hole auxiliary layer may be placed between the hole transport layer (or hole injection layer) and the light-emitting layer, and may exhibit an effect of facilitating or blocking the hole transport rate (or hole injection rate), and accordingly may adjust the charge balance. Also, the electron-blocking layer may be placed between the hole transport layer (or hole injection layer) and the light-emitting layer, and can confine the excitons within the light-emitting layer by blocking the overflow of electrons from the light-emitting layer to prevent a light-emitting leakage. When an organic electroluminescent device includes two or more hole transport layers, the hole transport layer additionally included may be used as a hole auxiliary layer or an electron-blocking layer. The light-emitting auxiliary layer, the hole auxiliary layer, or the electron-blocking layer may have an effect of improving the efficiency and/or lifetime of the organic electroluminescent device.

In addition, in an organic electroluminescent device of the present disclosure, a mixed region of an electron transport compound and a reductive dopant, or a mixed region of a hole transport compound and an oxidative dopant, may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferred oxidative dopants include various Lewis acids and acceptor compounds, and preferred reductive dopants include alkali metals, alkali metal compounds, alkaline earth metals, rare earth metals, and mixtures thereof. In addition, an organic electroluminescent device having at least two light-emitting layers and emitting white light may be manufactured by using the reductive dopant layer as a charge-generating layer.

The organic electroluminescent material according to one embodiment of the present disclosure may be used as a hole injection layer material for a white organic light-emitting device. In addition, the organic electroluminescent material according to one embodiment of the present disclosure may also be used in an organic electroluminescent device comprising a quantum dot (QD).

In order to form each layer of the organic electroluminescent device of the present disclosure, dry film-forming methods such as vacuum evaporation, sputtering, plasma, ion plating methods, etc. or wet film-forming methods such as inkjet printing, nozzle printing, slot coating, spin coating, dip coating, flow coating methods, etc. can be used.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film formation capability.

In addition, a display device, such as smartphones, tablets, notebooks, PCs, TVs, or display devices for vehicles, or lighting devices such as outdoor or indoor lighting, can be manufactured using the organic electroluminescent device of the present disclosure.

Hereinafter, for a detailed understanding of the present disclosure, the driving voltage and luminous efficiency of an OLED according to the present disclosure will be explained using a representative compound thereof. However, the following examples only describe the properties of the compound and the OLED according to the present disclosure, but the present disclosure is not limited to the following examples.

### Device Example 1: Producing a Red-Emitting OLED Having the Hole Injection Layer According to the Present Disclosure Deposited Thereon

An OLED according to the present disclosure was produced. First, a transparent electrode indium-tin-oxide (ITO) thin film (10 Ω/sq) on a glass substrate for an OLED (GEOMATEC CO., LTD., Japan) was subjected to an ultrasonic washing with acetone and isopropyl alcohol, sequentially, and was then stored in isopropyl alcohol. The ITO substrate was mounted on a substrate holder of a vacuum vapor deposition apparatus. Compound C-2 was introduced into a cell of the vacuum vapor deposition apparatus as a first hole injection compound, and Compound HI-2 was introduced into another cell. The two materials were evaporated at different rates, and Compound HI-2 was deposited in a doping amount of 3 wt % based to the total amount of Compound C-2 and Compound HI-2 to form a first hole injection layer with a thickness of 10 nm. Subsequently, Compound HT-1 as a first hole transport layer was deposited on the first hole injection layer to a thickness of 90 nm. Compound HT-2 was then introduced into another cell of the vacuum vapor deposition apparatus as a second hole transport compound, and was evaporated by applying an electric current to the cell, thereby depositing a second hole transport layer with a thickness of 60 nm on the first hole transport layer. Subsequently, Compound HT-3 was introduced into another cell of the vacuum vapor deposition apparatus as a third hole transport compound, and was evaporated by applying an electric current to the cell, thereby depositing a third hole transport layer with a thickness of 7.5 nm on the second hole transport layer. After formation of the hole injection layer and the hole transport layers, a light-emitting layer was deposited thereon as follows. After Compounds RH-1 and RH-2 and Compound RH-3 as light-emitting layer hosts were respectively introduced into two cells in a vacuum vapor deposition apparatus, Compound RH-1, Compound RH-2, and Compound RH-3 were evaporated at a rate ratio of 3.3:1.7:5, and Compound D-39 as a dopant was introduced into another cell and doped at 2 wt % based on the total amount of the hosts and the dopant, thereby forming a light-emitting layer with a thickness of 36 nm on the third hole transport layer. Subsequently, Compound B-3 as an electron buffer material was deposited to a thickness of 5 nm on the light-emitting layer. Subsequently, Compounds ET-2 and EI-1 as an electron transport layer were deposited to a thickness of 25 nm at a weight ratio of 2:1. After Compound EI-1 was deposited as an electron injection layer with a thickness of 2 nm on the electron transport layer, an Al cathode was deposited with a thickness of 80 nm on the electron injection layer using another vacuum vapor deposition apparatus to manufacture an OLED.

### Device Example 2: Producing a Red-Emitting OLED According to the Present Disclosure

In Device Example 2, an OLED was produced in the same manner as in Device Example 1, except that Compounds RH-4 and RH-5 as light-emitting layer hosts were respectively introduced into two cells in a vacuum vapor deposition apparatus, and Compounds RH-4 and RH-5 were evaporated at a rate ratio of 5:5.

### Device Example 3: Producing a Red-Emitting OLED According to the Present Disclosure

In Device Example 3, an OLED was produced in the same manner as in Device Example 1, except that Compound C-2 alone was deposited to a thickness of 10 nm as the first hole injection compound, without doping Compound HI-2.

### Device Example 4: Producing a Red-Emitting OLED According to the Present Disclosure

In Device Example 4, an OLED was produced in the same manner as in Device Example 2, except that Compound C-2 alone was deposited to a thickness of 10 nm as the first hole injection compound, without doping Compound HI-2.

### Comparative Example 1: Producing a Red-Emitting OLED not According to the Present Disclosure

In Comparative Example 1, an OLED was produced in the same manner as in Device Example 1, except that Compound HT-1 was used in the first hole injection layer instead of Compound C-2.

### Comparative Example 2: Producing a Red-Emitting OLED not According to the Present Disclosure

In Comparative Example 2, an OLED was produced in the same manner as in Device Example 2, except that Compound HT-1 was used in the first hole injection layer instead of Compound C-2.

### Comparative Example 3: Producing a Red-Emitting OLED not According to the Present Disclosure

In Comparative Example 3, an OLED was produced in the same manner as in Device Example 3, except that Compound HT-1 was used in the first hole injection layer instead of Compound C-2.

### Comparative Example 4: Producing a Red-Emitting OLED not According to the Present Disclosure

In Comparative Example 4, an OLED was produced in the same manner as in Device Example 4, except that Compound HT-1 was used in the first hole injection layer instead of Compound C-2.

The driving voltage, power efficiency, CIE color coordinates at a luminance of 1,000 nit, and the T95-converted lifetimes at a luminance of 10,000 nit (the time taken for luminance to reduce from 100% to 95%) of the organic electroluminescent devices according to Device Examples 1 and 2 and Comparative Examples 1 and 2 as described above were measured, and the results thereof are shown in Table 1 below.

TABLE 1

| | First hole | | Driving | Power | CIE color | Lifetime |
|---|---|---|---|---|---|---|
| | injection | Light-emitting | voltage | efficiency | coordinates | (T95) |
| | layer | layer Host | [V] | [lm/W] | (x, y) | [hr] |
| Device | C-2 | RH-1:RH-2:RH-3 | 3.2 | 30.5 | (0.663, 0.336) | 258 |
| Example 1 | | | | | | |
| Device | C-2 | RH-4:RH-5 | 3.3 | 32.0 | (0.662, 0.338) | 241 |
| Example 2 | | | | | | |
| Comparative | HT-1 | RH-1:RH-2:RH-3 | 3.6 | 26.2 | (0.663, 0.337) | 83 |
| Example 1 | | | | | | |
| Comparative | HT-1 | RH-4:RH-5 | 3.9 | 26.2 | (0.662, 0.338) | 59 |
| Example 2 | | | | | | |

From Table 1 above, it can be confirmed that the OLEDs according to the present disclosure exhibit a lower driving voltage, higher power efficiency, and longer lifetime characteristics compared to organic electroluminescent devices using a conventional compound in the first hole injection layer.

The driving voltage, luminous efficiency, power efficiency, and CIE color coordinates at a luminance of 1,000 nit of the organic electroluminescent devices according to Device Examples 3 and 4, and Comparative Examples 3 and 4 as described above were measured, and the results thereof are shown in Table 2 below.

TABLE 2

| | First hole | | Driving | Luminous | Power | CIE color |
|---|---|---|---|---|---|---|
| | injection | Light-emitting | voltage | efficiency | efficiency | coordinate |
| | layer | layer Host | [V] | [Cd/A] | [lm/W] | (x, y) |
| Device | C-2 | RH-1:RH-2:RH-3 | 8.8 | 20.7 | 7.4 | (0.656, 0.342) |
| Example 3 | | | | | | |
| Device | C-2 | RH-4:RH-5 | 9.3 | 21.3 | 7.2 | (0.656, 0.342) |
| Example 4 | | | | | | |
| Comparative | HT-1 | RH-1:RH-2:RH-3 | 11.6 | 12.0 | 3.3 | (0.652, 0.345) |
| Example 3 | | | | | | |
| Comparative | HT-1 | RH-4:RH-5 | 13.3 | 10.1 | 2.4 | (0.651, 0.346) |
| Example 4 | | | | | | |

From Table 2 above, it can be confirmed that the OLEDs according to the present disclosure exhibit a lower driving voltage, higher luminous efficiency, and higher power efficiency characteristics compared to organic electroluminescent devices using a conventional compound in the first hole injection layer.

### Device Example 5: Producing an OLED in which a Compound According to the Present Disclosure is Deposited

An OLED according to the present disclosure was produced. First, a transparent electrode indium-tin-oxide (ITO) thin film (10 Ω/sq) on a glass substrate for an OLED (GEOMATEC CO., LTD., Japan) was subjected to an ultrasonic washing with acetone and isopropyl alcohol, sequentially, and was then stored in isopropyl alcohol. After the ITO substrate was mounted on a substrate holder of a vacuum vapor deposition apparatus, Compound C-2 was introduced into a cell of the vacuum vapor deposition apparatus, and was evaporated to form a hole injection layer with a thickness of 10 nm. Subsequently, Compound HT-1 was introduced into another cell of the vacuum vapor deposition apparatus, and was evaporated by applying an electric current to the cell, thereby depositing a hole transport layer with a thickness of 80 nm on the hole injection layer. Compound EB-1 was evaporated onto the hole transport layer to deposit an electron-blocking layer with a thickness of 15 nm. After formation of the hole injection layer, the hole transport layer, and the electron-blocking layer, a light-emitting layer was deposited thereon as follows.

Host Compounds H-1 and H-2 described in Table 3 below were introduced into two cells of the vacuum vapor deposition apparatus as hosts, respectively, and Compound BD-100 was introduced into another cell as a dopant. The two host materials were then evaporated at a ratio of 1:1, and the dopant material was simultaneously evaporated at a different rate, and the dopant was deposited in a doping amount of 2 wt % based on the total amount of the hosts and dopant to form a light-emitting layer having a thickness of 18 nm on the electron-blocking layer. Subsequently, Compound B-1 was deposited as a buffer layer on the light-emitting layer to a thickness of 5 nm. Compounds ET-2 and EI-1 as an electron transport layer were deposited to a thickness of 25 nm at a weight ratio of 2:1. After Compound EI-2 was deposited as an electron injection layer with a thickness of 2 nm on the electron transport layer, an Al cathode was deposited with a thickness of 80 nm on the electron injection layer using another vacuum vapor deposition apparatus to manufacture an OLED. Each compound used for all of the materials was purified by vacuum sublimation under 10⁻⁶ Torr.

### Device Example 6: Producing an OLED in which a Compound According to the Present Disclosure is Deposited

An OLED was produced in the same manner as in Device Example 5, except that Compound D-2 was used in the formation of the hole injection layer.

### Comparative Example 5: Producing an OLED in which a Compound not According to the Present Disclosure is Deposited

An OLED was produced in the same manner as in Device Example 5, except that Compound HI-3 was used in the formation of the hole injection layer.

The driving voltage, luminous efficiency, and emission color at a luminance of 750 nit, and the time taken for luminance to reduce from 100% to 95% at a luminance of 1,280 nit (lifetime: T95) of the organic electroluminescent devices according to Device Examples 5, 6, and Comparative Example 5 as described above were measured, and the results thereof are shown in Table 3 below.

TABLE 3

| | Hole | Hole | Driving | Luminous | | |
|---|---|---|---|---|---|---|
| | injection | transport | voltage | efficiency | Emission | Lifetime |
| | layer | layer | [V] | [Cd/A] | color | (T95) [hr] |
| Device | C-2 | HT-1 | 4.32 | 6.0 | Blue | 2.7 |
| Example 5 | | | | | | |
| Device | D-2 | HT-1 | 4.17 | 6.1 | Blue | 16.2 |
| Example 6 | | | | | | |
| Comparative | HI-3 | HT-1 | 4.95 | 5.2 | Blue | 0.2 |
| Example 5 | | | | | | |

From Table 3 above, it can be confirmed that the OLEDs according to the present disclosure exhibit a lower driving voltage, higher luminous efficiency, and longer lifetime characteristics compared to organic electroluminescent devices using a conventional compound in the first hole injection layer.

The compounds used in Device Examples and Comparative Examples are shown in Table 4 below.

TABLE 4

| | |
|---|---|
| Hole Injection Layer/Hole Transport Layer | 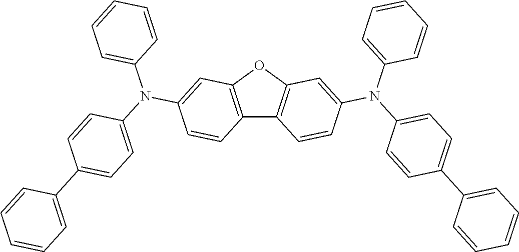 [SMILES: c1ccc(-c2ccc(N(c3ccccc3)c3ccc4c(c3)oc3cc(N(c5ccccc5)c5ccc(-c6ccccc6)cc5)ccc34)cc2)cc1] |
| | C-2 |
| | 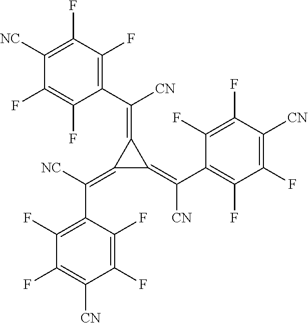 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-2 |
| | 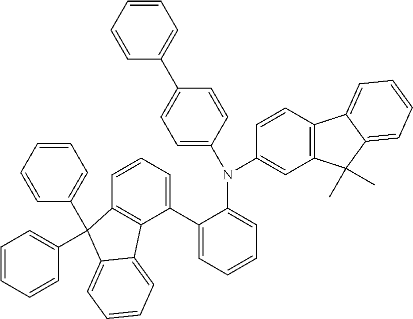 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21] |
| | HT-1 |
| | 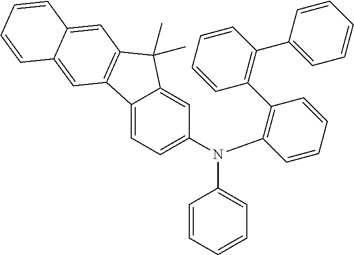 [SMILES: CC1(C)c2cc(N(c3ccccc3)c3ccccc3-c3ccccc3-c3ccccc3)ccc2-c2cc3ccccc3cc21] |
| | HT-2 |
| | 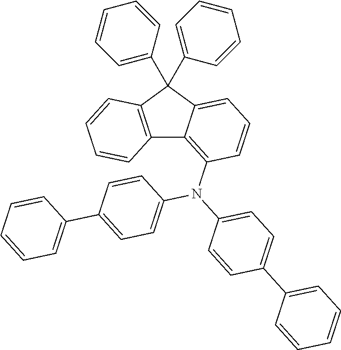 [SMILES: c1ccc(-c2ccc(N(c3ccc(-c4ccccc4)cc3)c3cccc4c3-c3ccccc3C4(c3ccccc3)c3ccccc3)cc2)cc1] |
| | HT-3 |
| | 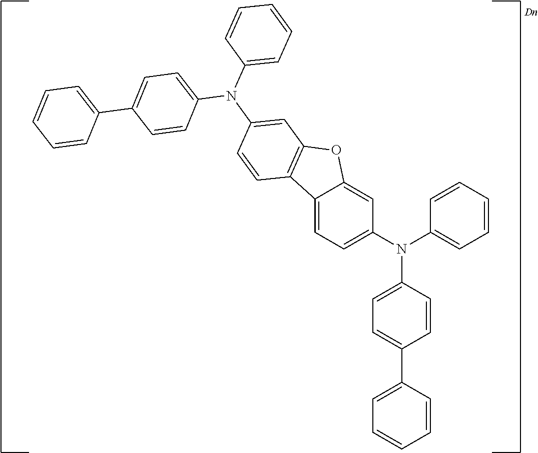 |
| | D-2 |
| | 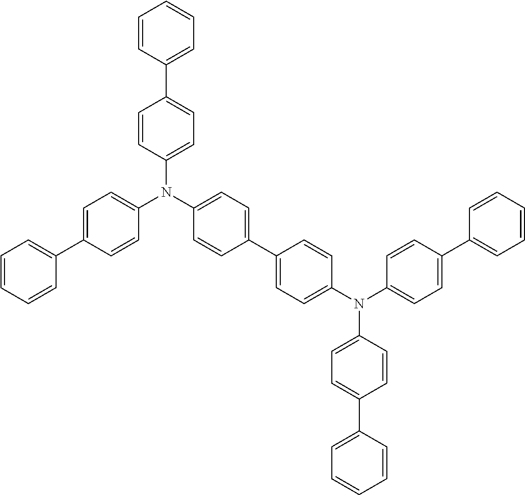 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1] |
| | HI-3 |
| Electron- Blocking Layer | 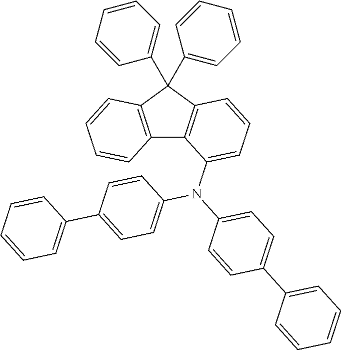 [SMILES: c1ccc(-c2ccc(N(c3ccc(-c4ccccc4)cc3)c3cccc4c3-c3ccccc3C4(c3ccccc3)c3ccccc3)cc2)cc1] |
| | EB-1 |
| Light-Emitting Layer | 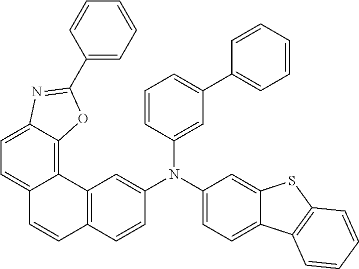 [SMILES: c1ccc(-c2cccc(N(c3ccc4c(c3)sc3ccccc34)c3ccc4ccc5ccc6nc(-c7ccccc7)oc6c5c4c3)c2)cc1] |
| | RH-1 |
| | 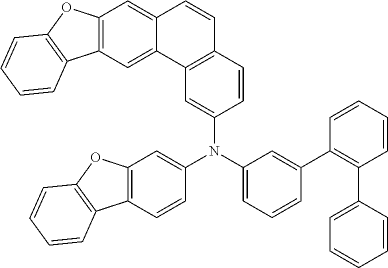 [SMILES: c1ccc(-c2ccccc2-c2cccc(N(c3ccc4c(c3)oc3ccccc34)c3ccc4ccc5cc6oc7ccccc7c6cc5c4c3)c2)cc1] |
| | RH-2 |
| | 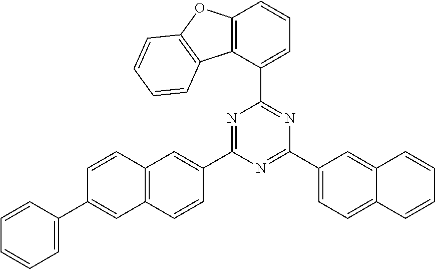 [SMILES: c1ccc(-c2ccc3cc(-c4nc(-c5ccc6ccccc6c5)nc(-c5cccc6oc7ccccc7c56)n4)ccc3c2)cc1] |
| | RH-3 |
| | 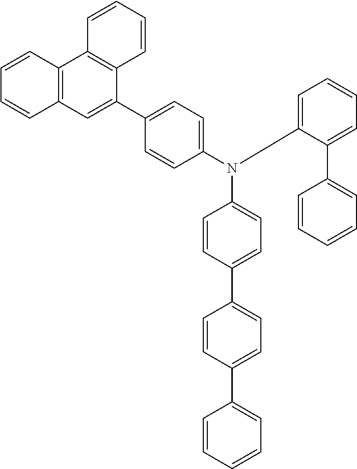 [SMILES: c1ccc(-c2ccc(-c3ccc(N(c4ccc(-c5cc6ccccc6c6ccccc56)cc4)c4ccccc4-c4ccccc4)cc3)cc2)cc1] |
| | RH-4 |
| | 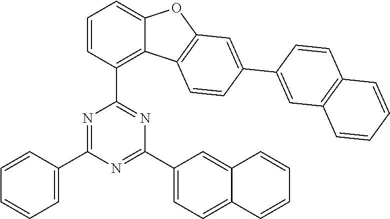 [SMILES: c1ccc(-c2nc(-c3ccc4ccccc4c3)nc(-c3cccc4oc5cc(-c6ccc7ccccc7c6)ccc5c34)n2)cc1] |
| | RH-5 |
| | 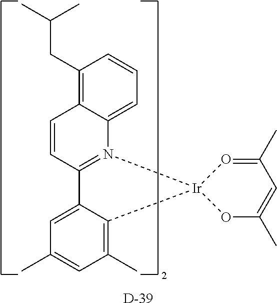 |
| | 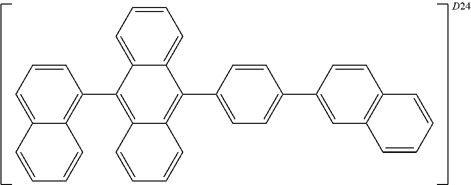 |
| | H-1 |
| | 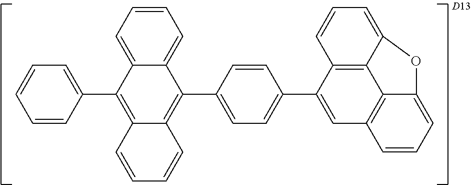 |
| | H-2 |
| | 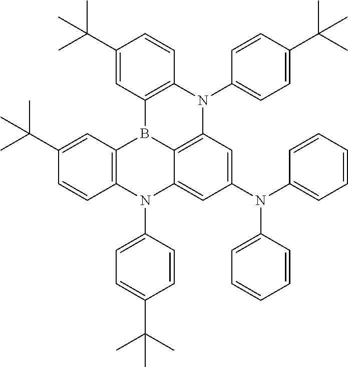 [SMILES: CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1] |
| | BD-100 |
| Electron Buffer Layer | 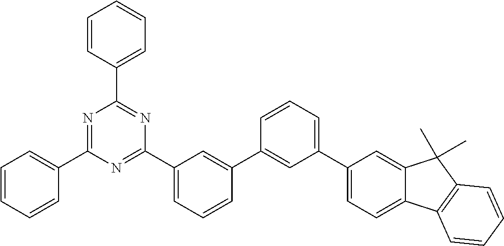 [SMILES: CC1(C)c2ccccc2-c2ccc(-c3cccc(-c4cccc(-c5nc(-c6ccccc6)nc(-c6ccccc6)n5)c4)c3)cc21] |
| | B-3 |
| | 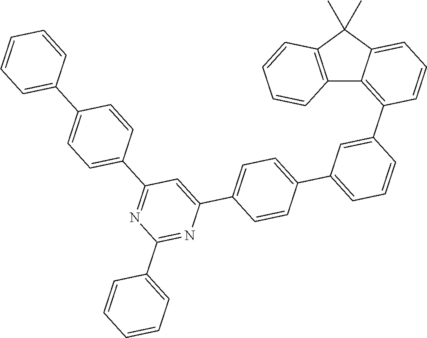 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | B-1 |
| Electron Transport Layer/ Electron Injection Layer | 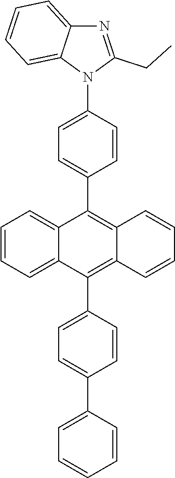 [SMILES: CCc1nc2ccccc2n1-c1ccc(-c2c3ccccc3c(-c3ccc(-c4ccccc4)cc3)c3ccccc23)cc1] |
| | ET-2 |
| | 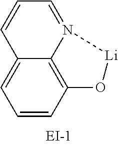 |
| | Yb |
| | EI-2 |

1. An organic electroluminescent device comprising a first electrode; a second electrode facing the first electrode; and an organic layer disposed between the first electrode and the second electrode, wherein the organic layer comprises a hole transport region, a light-emitting layer, and an electron transport region, wherein the hole transport region comprises a hole injection layer and a plurality of hole transport layers, wherein the hole injection layer comprises a compound represented by the following Formula 1, wherein each of the plurality of hole transport layers comprises at least one compound, and wherein at least two of the plurality of hole transport layers comprise different compounds from each other: 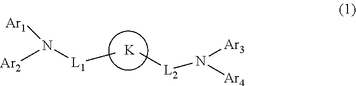
wherein in Formula 1,
ring K is selected from the following Formulas 1-1 to 1-4; 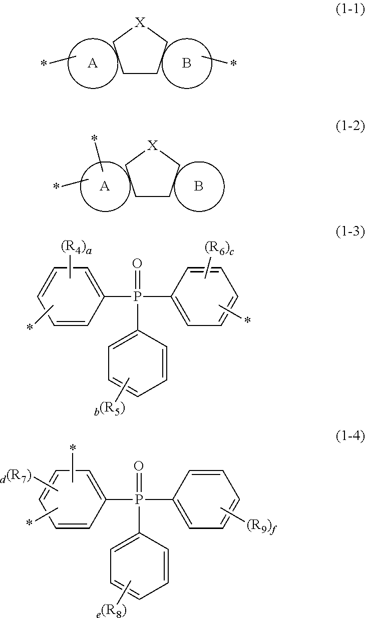
wherein in Formulas 1, and 1-1 to 1-4,
X represents —N(R₁)—, —O—, —S—, —C(R₂)(R₃)—, —Se—, —Si(R₂)(R₃)—, or —P(═O)—;
ring A and ring B each independently represent a substituted or unsubstituted (C₆-C₃₀)arene, or a substituted or unsubstituted (3- to 30-membered)heteroarene;
R₁ to R₃ each independently represent a substituted or unsubstituted (C₆-C₃₀)aryl, a substituted or unsubstituted (C₁-C₃₀)alkyl, a substituted or unsubstituted (C₃-C₃₀)cycloalkyl, or a substituted or unsubstituted (3- to 30-membered)heteroaryl, or may be linked to an adjacent substituent(s) to form a substituted or unsubstituted (C₃-C₃₀)mono- or polycyclic, alicyclic, aromatic ring, or a combination thereof, wherein the carbon atoms of the formed ring may be replaced with at least one heteroatom selected from nitrogen, oxygen, and sulfur;
R₄ to R₉ and Ar₁ to Ar₄ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀)aliphatic ring(s) and a (C₆-C₃₀)aromatic ring(s), a substituted or unsubstituted mono- or di(C₁-C₃₀)alkylamino, a substituted or unsubstituted mono- or di(C₂-C₃₀)alkenylamino, a substituted or unsubstituted mono- or di(C₆-C₃₀)arylamino, a substituted or unsubstituted mono- or di(3- to 30-membered)heteroarylamino, a substituted or unsubstituted (C₁-C₃₀)alkyl(C₂-C₃₀)alkenylamino, a substituted or unsubstituted (C₁-C₃₀)alkyl(C₆-C₃₀)arylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, or a substituted or unsubstituted (C₆-C₃₀)aryl(3- to 30-membered)heteroarylamino, or may be linked to an adjacent substituent(s) to form a substituted or unsubstituted (C₃-C₃₀)mono- or polycyclic, alicyclic, aromatic ring, or a combination thereof, wherein the carbon atoms of the formed ring may be replaced with at least one heteroatom selected from nitrogen, oxygen, and sulfur;
L₁ and L₂ each independently represent a single bond, a substituted or unsubstituted (C₆-C₃₀)arylene, or a substituted or unsubstituted (3- to 30-membered)heteroarylene;
a and c each represent an integer of 1 to 4, b, e, and f each represent an integer of 1 to 5, and d represents an integer of 1 to 3; and
the heteroaryl(ene) comprises at least one heteroatom selected from B, N, O, S, Si, Se, and P.

2. The organic electroluminescent device according to claim 1, wherein at least one of the plurality of hole transport layers comprises a compound represented by the following Formula 2: 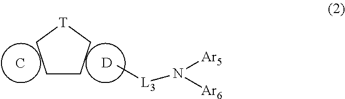
wherein in Formula 2,
T represents —N(R₅₁)—, —C(R₅₂)(R₅₃)—, —Si(R₅₄)(R₅₅)—, —Se—, —O—, or —S—;
ring C or ring D each independently represents a substituted or unsubstituted (C₆-C₃₀)arene, or a substituted or unsubstituted (3- to 30-membered)heteroarene;
R₅₁ to R₅₅ each independently represent a substituted or unsubstituted (C₆-C₃₀)aryl, a substituted or unsubstituted (C₁-C₃₀)alkyl, a substituted or unsubstituted (C₃-C₃₀)cycloalkyl, or a substituted or unsubstituted (3- to 30-membered)heteroaryl, or may be linked to an adjacent substituent(s) to form a substituted or unsubstituted (C₃-C₃₀)mono- or polycyclic, alicyclic, aromatic ring, or a combination thereof, wherein the carbon atoms of the formed ring may be replaced with at least one heteroatom selected from nitrogen, oxygen, and sulfur;
Ar₅ and Ar₆ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀)aliphatic ring(s) and a (C₆-C₃₀)aromatic ring(s), a substituted or unsubstituted mono- or di(C₁-C₃₀)alkylamino, a substituted or unsubstituted mono- or di(C₂-C₃₀)alkenylamino, a substituted or unsubstituted mono- or di(C₆-C₃₀)arylamino, a substituted or unsubstituted mono- or di(3- to 30-membered)heteroarylamino, a substituted or unsubstituted (C₁-C₃₀)alkyl(C₂-C₃₀)alkenylamino, a substituted or unsubstituted (C₁-C₃₀)alkyl(C₆-C₃₀)arylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, or a substituted or unsubstituted (C₆-C₃₀)aryl(3- to 30-membered)heteroarylamino, or may be linked to an adjacent substituent(s) to form a substituted or unsubstituted (C₃-C₃₀)mono- or polycyclic, alicyclic, aromatic ring, or a combination thereof, wherein the carbon atoms of the formed ring may be replaced with at least one heteroatom selected from nitrogen, oxygen, and sulfur;
L₃ represents a single bond, a substituted or unsubstituted (C₆-C₃₀)arylene, or a substituted or unsubstituted (3- to 30-membered)heteroarylene; and
the heteroaryl(ene) comprises at least one heteroatom selected from B, N, O, S, Si, Se, and P.

3. The organic electroluminescent device according to claim 1 or 2, wherein the substituted alkyl, the substituted aryl, the substituted arylene, the substituted arene, the substituted heteroaryl, the substituted heteroarylene, the substituted heteroarene, the substituted cycloalkyl, the substituted alkoxy, the substituted trialkylsilyl, the substituted dialkylarylsilyl, the substituted alkyldiarylsilyl, the substituted triarylsilyl, the substituted fused ring group of an aliphatic ring(s) and an aromatic ring(s), the substituted mono- or dialkylamino, the substituted mono- or dialkenylamino, the substituted mono- or diarylamino, the substituted mono- or diheteroarylamino, the substituted alkylalkenylamino, the substituted alkylarylamino, the substituted alkylheteroarylamino, the substituted alkenylarylamino, the substituted alkenylheteroarylamino, the substituted arylheteroarylamino, the substituted mono- or polycyclic, alicyclic, aromatic ring, or a combination thereof each independently are substituted with at least one selected from the group consisting of deuterium, a halogen, a cyano, a carboxyl, a nitro, a hydroxy, phosphinoxide, a (C₁-C₃₀)alkyl, a halo(C₁-C₃₀)alkyl, a (C₂-C₃₀)alkenyl, a (C₂-C₃₀)alkynyl, a (C₁-C₃₀)alkoxy, a (C₁-C₃₀)alkylthio, a (C₃-C₃₀)cycloalkyl, a (C₃-C₃₀)cycloalkenyl, a (3- to 7-membered)heterocycloalkyl, a (C₆-C₃₀)aryloxy, a (C₆-C₃₀)arylthio, a (3- to 30-membered)heteroaryl, a (C₆-C₃₀)aryl, a tri(C₁-C₃₀)alkylsilyl, a tri(C₆-C₃₀)arylsilyl, a di(C₁-C₃₀)alkyl(C₆-C₃₀)arylsilyl, a (C₁-C₃₀)alkyldi(C₆-C₃₀)arylsilyl, a fused ring group of a (C₃-C₃₀)aliphatic ring(s) and a (C₆-C₃₀)aromatic ring(s), an amino, a mono- or di(C₁-C₃₀)alkylamino, a mono- or di(C₂-C₃₀)alkenylamino, (C₁-C₃₀)alkyl(C₂-C₃₀)alkenylamino, a mono- or di(C₆-C₃₀)arylamino, a (C₁-C₃₀)alkyl(C₆-C₃₀)arylamino, a mono- or di(3- to 30-membered)heteroarylamino, a (C₁-C₃₀)alkyl(3- to 30-membered)heteroarylamino, a (C₂-C₃₀)alkenyl(C₆-C₃₀)arylamino, a (C₂-C₃₀)alkenyl(3- to 30-membered)heteroarylamino, a (C₆-C₃₀)aryl(3- to 30-membered)heteroarylamino, a (C₁-C₃₀)alkylcarbonyl, a (C₁-C₃₀)alkoxycarbonyl, a (C₆-C₃₀)arylcarbonyl, a (C₆-C₃₀)arylphosphinyl, a di(C₆-C₃₀)arylboronyl, a di(C₁-C₃₀)alkylboronyl, a (C₁-C₃₀)alkyl(C₆-C₃₀)arylboronyl, a (C₆-C₃₀)aryl(C₁-C₃₀)alkyl, a (C₁-C₃₀)alkyl(C₆-C₃₀)aryl, and combinations thereof.

4. The organic electroluminescent device according to claim 1, wherein the hole injection layer further comprises at least one p-dopant.

5. The organic electroluminescent device according to claim 1, wherein the hole injection layer does not comprise a p-dopant.

6. The organic electroluminescent device according to claim 1, wherein the plurality of hole transport layers comprises a first hole transport layer, a second hole transport layer, and a third hole transport layer.

7. The organic electroluminescent device according to claim 1, wherein the plurality of hole transport layers comprises at least three layers, wherein the plurality of hole transport layers comprises a first hole transport layer, a second hole transport layer, and a third hole transport layer, and wherein a compound comprised in the first hole transport layer, a compound comprised in the second hole transport layer, and a compound comprised in the third hole transport layer are each different from one another.

8. The organic electroluminescent device according to claim 1, wherein the ring A and the ring B are each independently selected from the following Formulas 1-5 to 1-9: 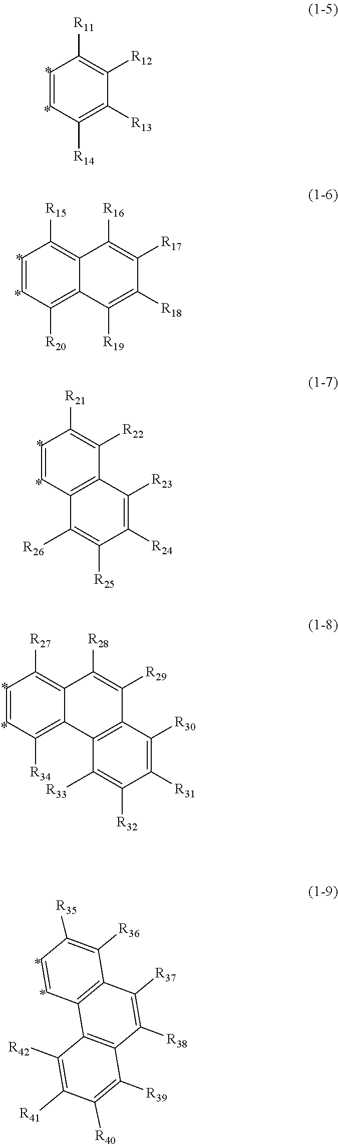
wherein in Formulas 1-5 to 1-9,
R₁₁ to R₄₂ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀)aliphatic ring(s) and a (C₆-C₃₀)aromatic ring(s), a substituted or unsubstituted mono- or di(C₁-C₃₀)alkylamino, a substituted or unsubstituted mono- or di(C₂-C₃₀)alkenylamino, a substituted or unsubstituted mono- or di(C₆-C₃₀)arylamino, a substituted or unsubstituted mono- or di(3- to 30-membered)heteroarylamino, a substituted or unsubstituted (C₁-C₃₀)alkyl(C₂-C₃₀)alkenylamino, a substituted or unsubstituted (C₁-C₃₀)alkyl(C₆-C₃₀)arylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, or a substituted or unsubstituted (C₆-C₃₀)aryl(3- to 30-membered)heteroarylamino, or may be linked to an adjacent substituent(s) to form a substituted or unsubstituted (C₃-C₃₀)mono- or polycyclic, alicyclic, aromatic ring, or a combination thereof, wherein the carbon atoms of the formed ring may be replaced with at least one heteroatom selected from nitrogen, oxygen, and sulfur.

9. The organic electroluminescent device according to claim 2, wherein the ring C and the ring D are each independently selected from the following Formulas 1-5 to 1-9: 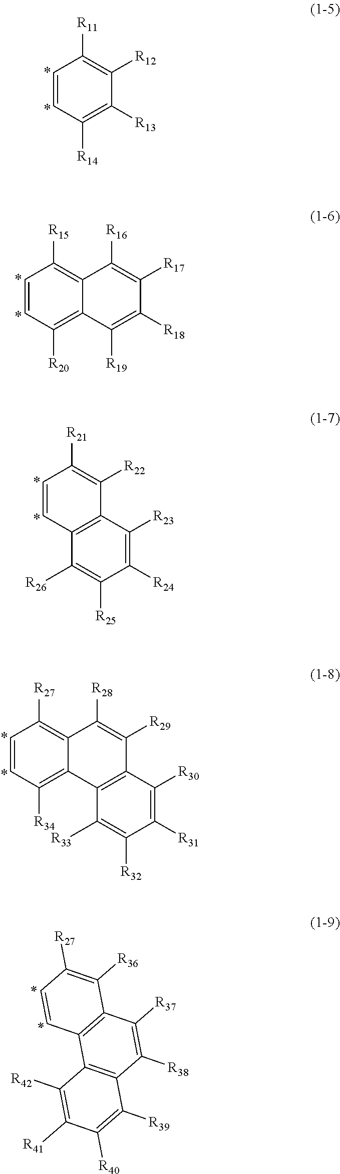
wherein in Formulas 1-5 to 1-9,
R₁₁ to R₄₂ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀)aliphatic ring(s) and a (C₆-C₃₀)aromatic ring(s), a substituted or unsubstituted mono- or di(C₁-C₃₀)alkylamino, a substituted or unsubstituted mono- or di(C₂-C₃₀)alkenylamino, a substituted or unsubstituted mono- or di(C₆-C₃₀)arylamino, a substituted or unsubstituted mono- or di(3- to 30-membered)heteroarylamino, a substituted or unsubstituted (C₁-C₃₀)alkyl(C₂-C₃₀)alkenylamino, a substituted or unsubstituted (C₁-C₃₀)alkyl(C₆-C₃₀)arylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, or a substituted or unsubstituted (C₆-C₃₀)aryl(3- to 30-membered)heteroarylamino, or may be linked to an adjacent substituent(s) to form a substituted or unsubstituted (C₃-C₃₀)mono- or polycyclic, alicyclic, aromatic ring, or a combination thereof, and wherein the carbon atoms of the formed ring may be replaced with at least one heteroatom selected from nitrogen, oxygen, and sulfur.

10. The organic electroluminescent device according to claim 1, wherein the light-emitting layer comprises a plurality of light-emitting layers comprising at least two layers, and wherein a first light-emitting layer and a second light-emitting layer are comprised adjacently.

11. The organic electroluminescent device according to claim 1, wherein the light-emitting layer comprises a plurality of light-emitting layers comprising at least three layers, and wherein a first light-emitting layer, a second light-emitting layer, and a third light-emitting layer are comprised sequentially adjacent to one another.

12. The organic electroluminescent device according to claim 1, wherein the compound represented by Formula 1 is at least one selected from the following compounds: 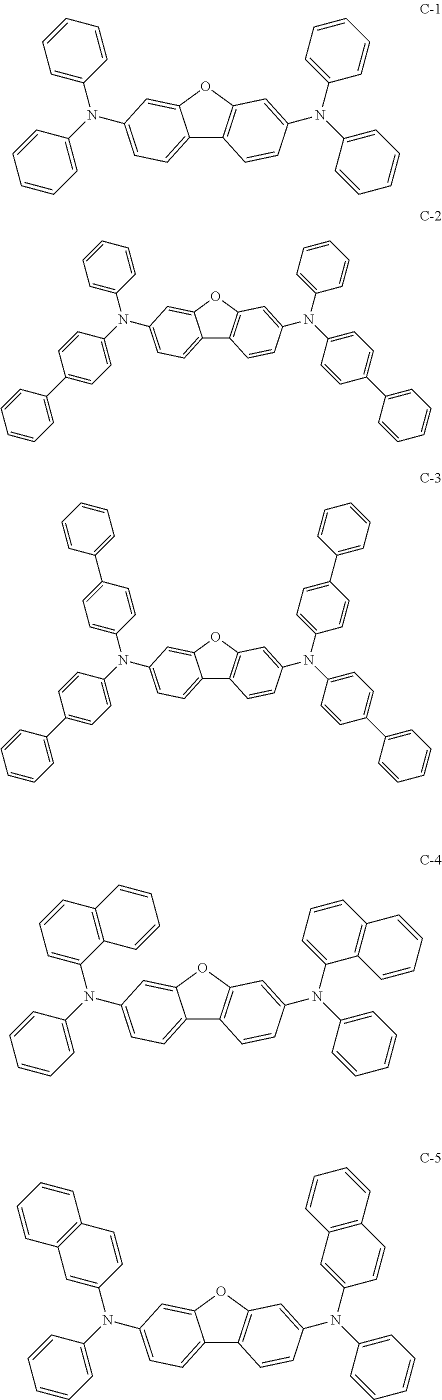 [SMILES: c1ccc(N(c2ccccc2)c2ccc3c(c2)oc2cc(N(c4ccccc4)c4ccccc4)ccc23)cc1 ; c1ccc(-c2ccc(N(c3ccccc3)c3ccc4c(c3)oc3cc(N(c5ccccc5)c5ccc(-c6ccccc6)cc5)ccc34)cc2)cc1 ; c1ccc(-c2ccc(N(c3ccc(-c4ccccc4)cc3)c3ccc4c(c3)oc3cc(N(c5ccc(-c6ccccc6)cc5)c5ccc(-c6ccccc6)cc5)ccc34)cc2)cc1 ; c1ccc(N(c2ccc3c(c2)oc2cc(N(c4ccccc4)c4cccc5ccccc45)ccc23)c2cccc3ccccc23)cc1 ; c1ccc(N(c2ccc3ccccc3c2)c2ccc3c(c2)oc2cc(N(c4ccccc4)c4ccc5ccccc5c4)ccc23)cc1] 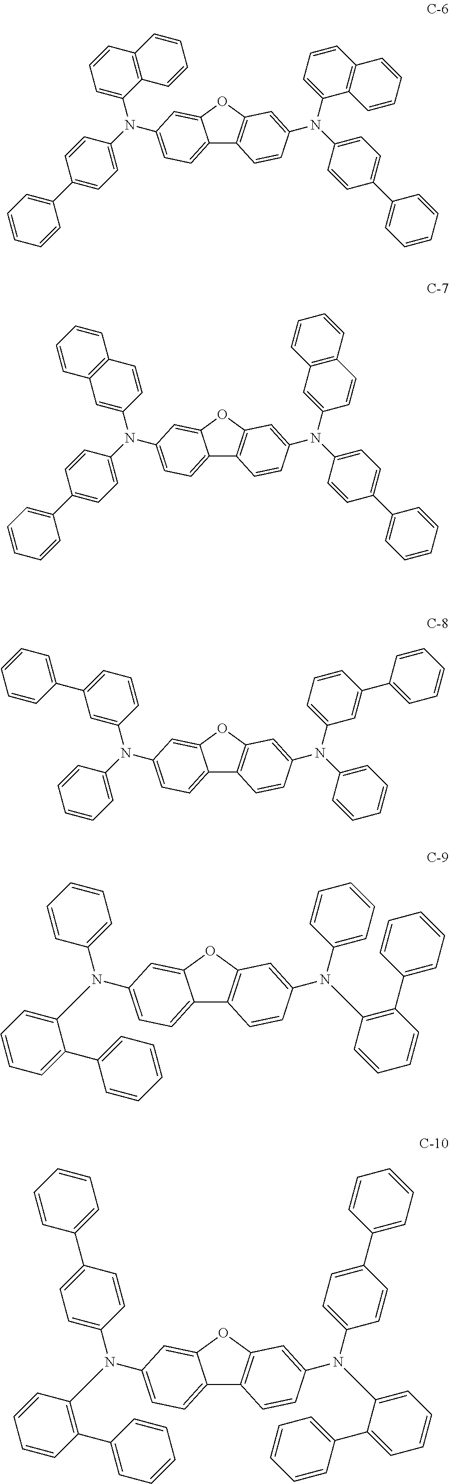 [SMILES: c1ccc(-c2ccc(N(c3ccc4c(c3)oc3cc(N(c5ccc(-c6ccccc6)cc5)c5cccc6ccccc56)ccc34)c3cccc4ccccc34)cc2)cc1 ; c1ccc(-c2ccc(N(c3ccc4ccccc4c3)c3ccc4c(c3)oc3cc(N(c5ccc(-c6ccccc6)cc5)c5ccc6ccccc6c5)ccc34)cc2)cc1 ; c1ccc(-c2cccc(N(c3ccccc3)c3ccc4c(c3)oc3cc(N(c5ccccc5)c5cccc(-c6ccccc6)c5)ccc34)c2)cc1 ; c1ccc(-c2ccccc2N(c2ccccc2)c2ccc3c(c2)oc2cc(N(c4ccccc4)c4ccccc4-c4ccccc4)ccc23)cc1 ; c1ccc(-c2ccc(N(c3ccc4c(c3)oc3cc(N(c5ccc(-c6ccccc6)cc5)c5ccccc5-c5ccccc5)ccc34)c3ccccc3-c3ccccc3)cc2)cc1] 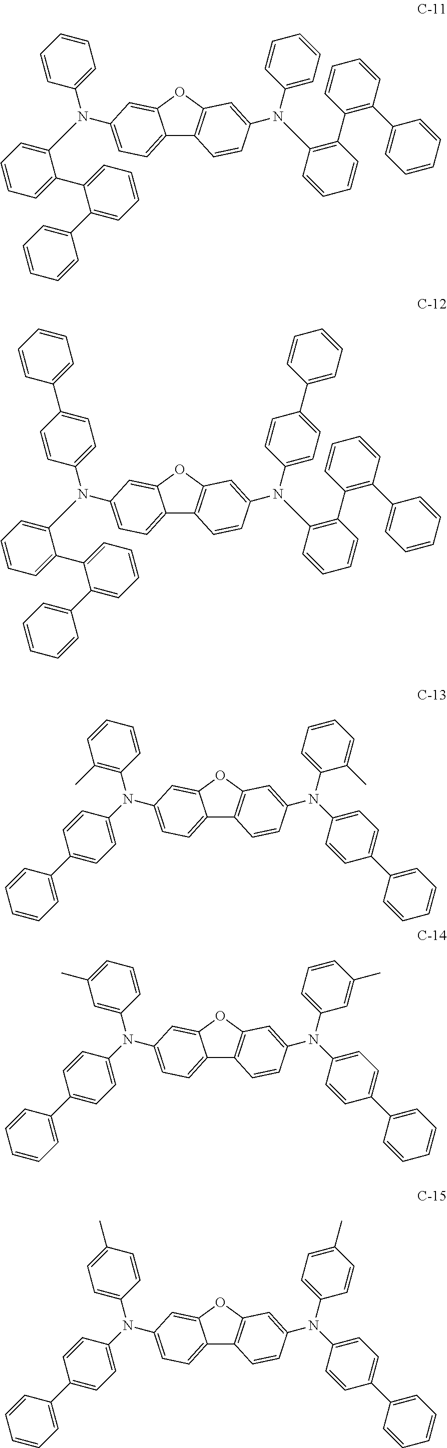 [SMILES: c1ccc(-c2ccccc2-c2ccccc2N(c2ccccc2)c2ccc3c(c2)oc2cc(N(c4ccccc4)c4ccccc4-c4ccccc4-c4ccccc4)ccc23)cc1 ; c1ccc(-c2ccc(N(c3ccc4c(c3)oc3cc(N(c5ccc(-c6ccccc6)cc5)c5ccccc5-c5ccccc5-c5ccccc5)ccc34)c3ccccc3-c3ccccc3-c3ccccc3)cc2)cc1 ; Cc1ccccc1N(c1ccc(-c2ccccc2)cc1)c1ccc2c(c1)oc1cc(N(c3ccc(-c4ccccc4)cc3)c3ccccc3C)ccc12 ; Cc1cccc(N(c2ccc(-c3ccccc3)cc2)c2ccc3c(c2)oc2cc(N(c4ccc(-c5ccccc5)cc4)c4cccc(C)c4)ccc23)c1 ; Cc1ccc(N(c2ccc(-c3ccccc3)cc2)c2ccc3c(c2)oc2cc(N(c4ccc(C)cc4)c4ccc(-c5ccccc5)cc4)ccc23)cc1] 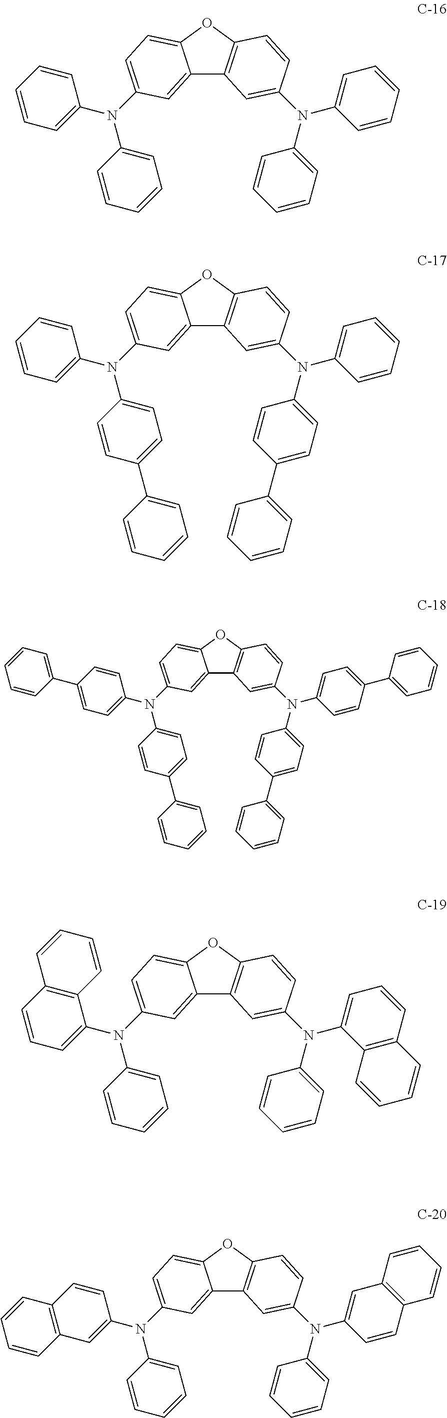 [SMILES: c1ccc(N(c2ccccc2)c2ccc3oc4ccc(N(c5ccccc5)c5ccccc5)cc4c3c2)cc1 ; c1ccc(-c2ccc(N(c3ccccc3)c3ccc4oc5ccc(N(c6ccccc6)c6ccc(-c7ccccc7)cc6)cc5c4c3)cc2)cc1 ; c1ccc(-c2ccc(N(c3ccc(-c4ccccc4)cc3)c3ccc4oc5ccc(N(c6ccc(-c7ccccc7)cc6)c6ccc(-c7ccccc7)cc6)cc5c4c3)cc2)cc1 ; c1ccc(N(c2ccc3oc4ccc(N(c5ccccc5)c5cccc6ccccc56)cc4c3c2)c2cccc3ccccc23)cc1 ; c1ccc(N(c2ccc3ccccc3c2)c2ccc3oc4ccc(N(c5ccccc5)c5ccc6ccccc6c5)cc4c3c2)cc1] 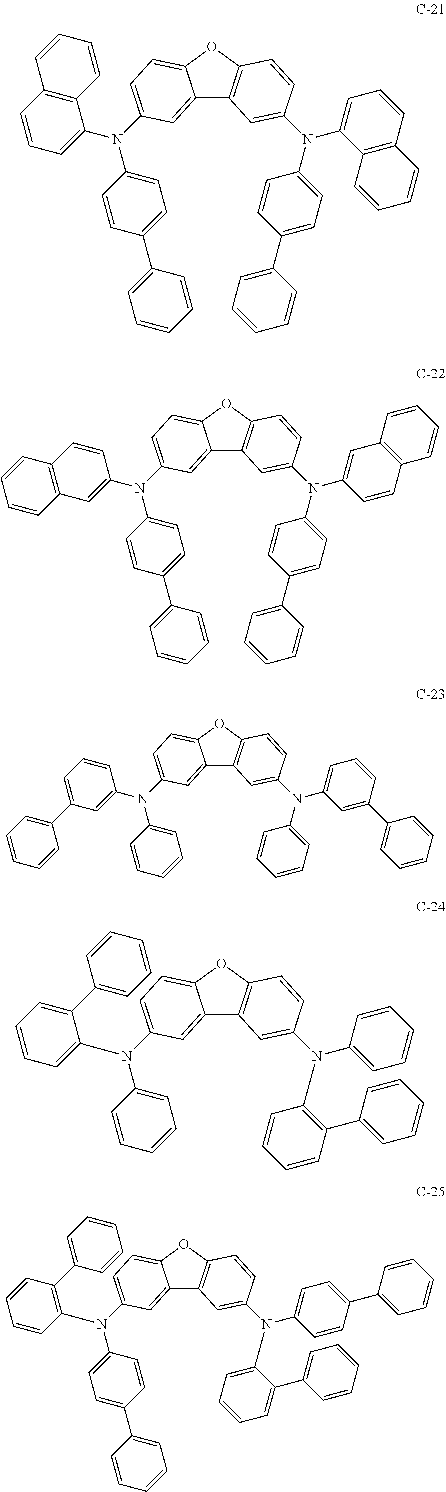 [SMILES: c1ccc(-c2ccc(N(c3ccc4oc5ccc(N(c6ccc(-c7ccccc7)cc6)c6cccc7ccccc67)cc5c4c3)c3cccc4ccccc34)cc2)cc1 ; c1ccc(-c2ccc(N(c3ccc4ccccc4c3)c3ccc4oc5ccc(N(c6ccc(-c7ccccc7)cc6)c6ccc7ccccc7c6)cc5c4c3)cc2)cc1 ; c1ccc(-c2cccc(N(c3ccccc3)c3ccc4oc5ccc(N(c6ccccc6)c6cccc(-c7ccccc7)c6)cc5c4c3)c2)cc1 ; c1ccc(-c2ccccc2N(c2ccccc2)c2ccc3oc4ccc(N(c5ccccc5)c5ccccc5-c5ccccc5)cc4c3c2)cc1 ; c1ccc(-c2ccc(N(c3ccc4oc5ccc(N(c6ccc(-c7ccccc7)cc6)c6ccccc6-c6ccccc6)cc5c4c3)c3ccccc3-c3ccccc3)cc2)cc1] 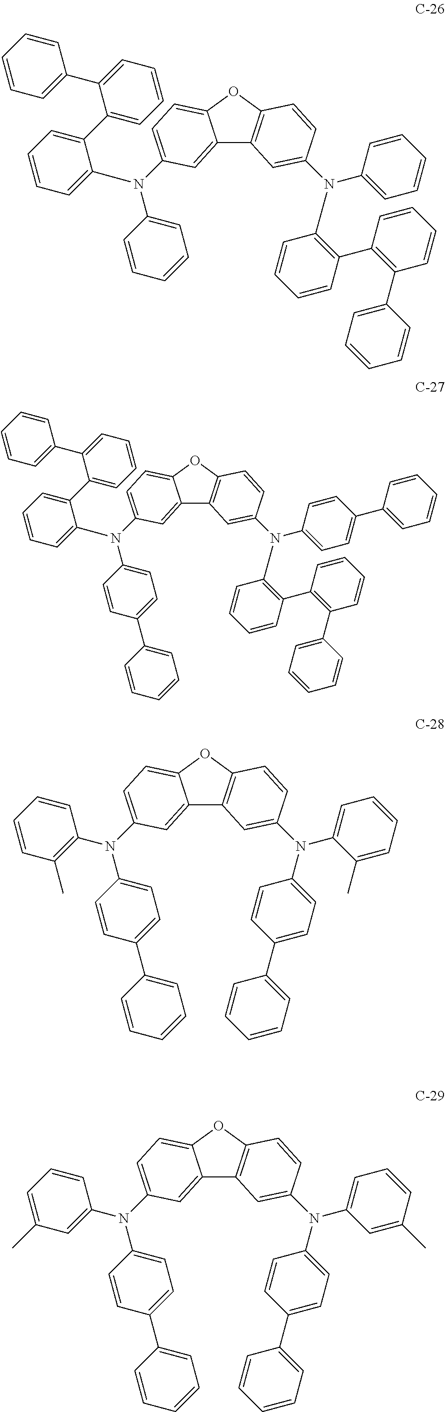 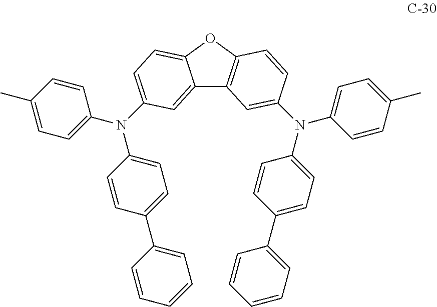 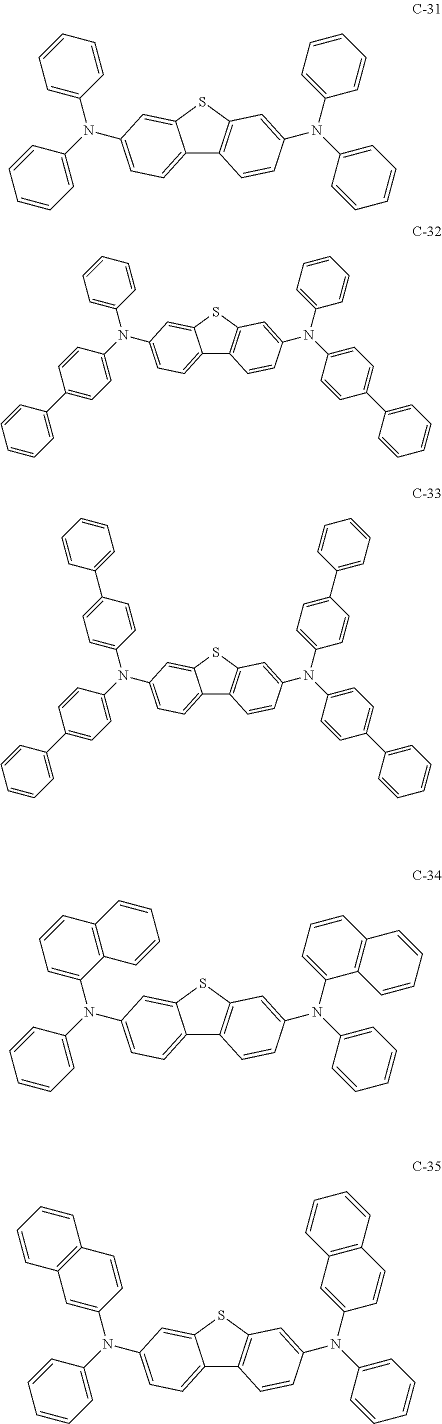 [SMILES: c1ccc(N(c2ccccc2)c2ccc3c(c2)sc2cc(N(c4ccccc4)c4ccccc4)ccc23)cc1 ; c1ccc(-c2ccc(N(c3ccccc3)c3ccc4c(c3)sc3cc(N(c5ccccc5)c5ccc(-c6ccccc6)cc5)ccc34)cc2)cc1 ; c1ccc(-c2ccc(N(c3ccc(-c4ccccc4)cc3)c3ccc4c(c3)sc3cc(N(c5ccc(-c6ccccc6)cc5)c5ccc(-c6ccccc6)cc5)ccc34)cc2)cc1 ; c1ccc(N(c2ccc3c(c2)sc2cc(N(c4ccccc4)c4cccc5ccccc45)ccc23)c2cccc3ccccc23)cc1 ; c1ccc(N(c2ccc3ccccc3c2)c2ccc3c(c2)sc2cc(N(c4ccccc4)c4ccc5ccccc5c4)ccc23)cc1] 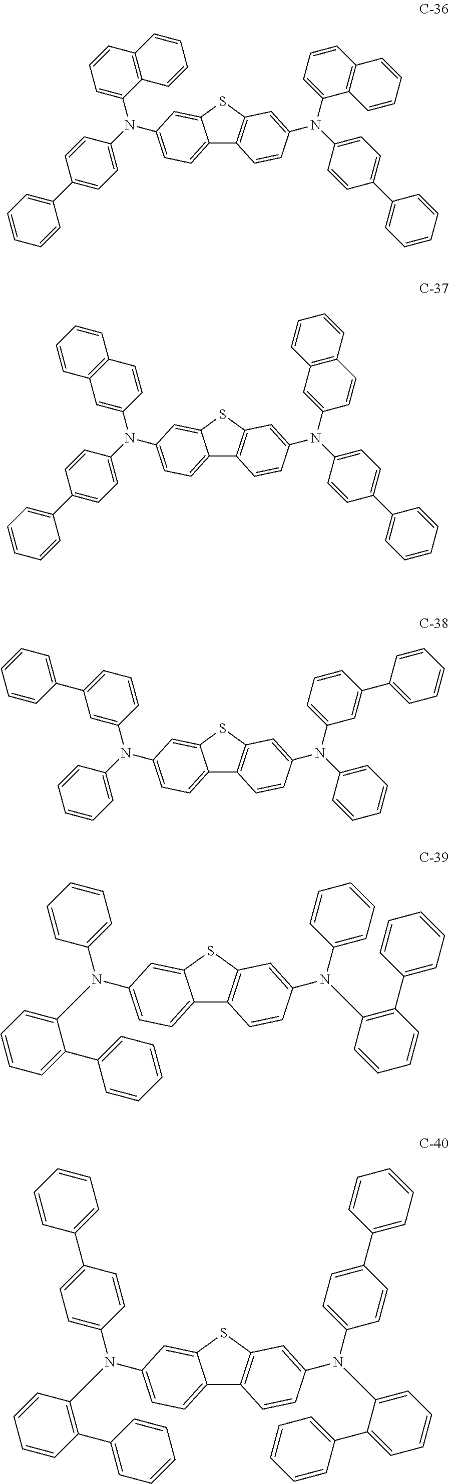 [SMILES: c1ccc(-c2ccc(N(c3ccc4c(c3)sc3cc(N(c5ccc(-c6ccccc6)cc5)c5cccc6ccccc56)ccc34)c3cccc4ccccc34)cc2)cc1 ; c1ccc(-c2ccc(N(c3ccc4ccccc4c3)c3ccc4c(c3)sc3cc(N(c5ccc(-c6ccccc6)cc5)c5ccc6ccccc6c5)ccc34)cc2)cc1 ; c1ccc(-c2cccc(N(c3ccccc3)c3ccc4c(c3)sc3cc(N(c5ccccc5)c5cccc(-c6ccccc6)c5)ccc34)c2)cc1 ; c1ccc(-c2ccccc2N(c2ccccc2)c2ccc3c(c2)sc2cc(N(c4ccccc4)c4ccccc4-c4ccccc4)ccc23)cc1 ; c1ccc(-c2ccc(N(c3ccc4c(c3)sc3cc(N(c5ccc(-c6ccccc6)cc5)c5ccccc5-c5ccccc5)ccc34)c3ccccc3-c3ccccc3)cc2)cc1] 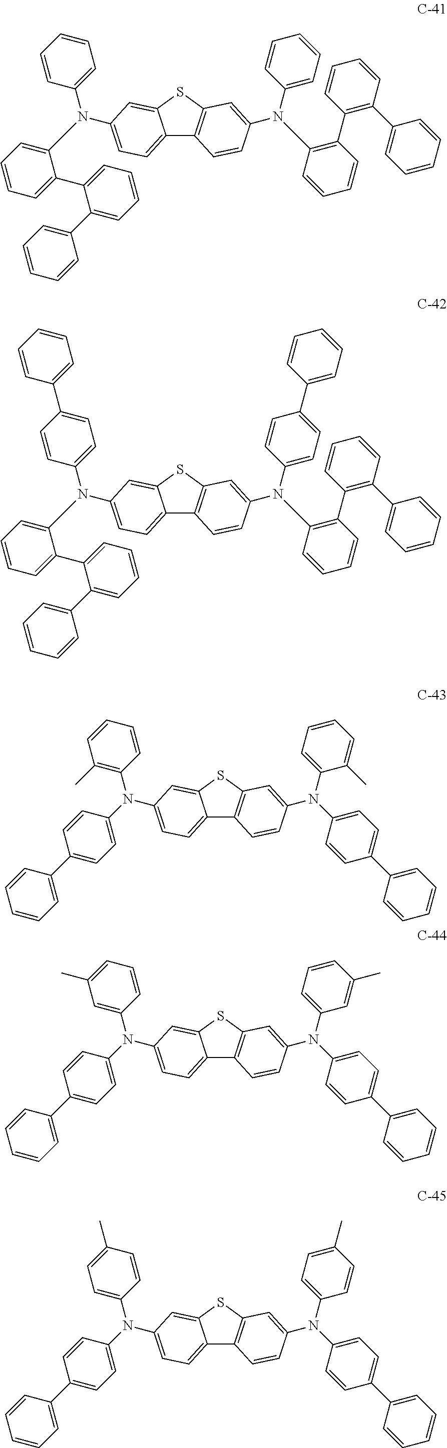 [SMILES: c1ccc(-c2ccccc2-c2ccccc2N(c2ccccc2)c2ccc3c(c2)sc2cc(N(c4ccccc4)c4ccccc4-c4ccccc4-c4ccccc4)ccc23)cc1 ; c1ccc(-c2ccc(N(c3ccc4c(c3)sc3cc(N(c5ccc(-c6ccccc6)cc5)c5ccccc5-c5ccccc5-c5ccccc5)ccc34)c3ccccc3-c3ccccc3-c3ccccc3)cc2)cc1 ; Cc1ccccc1N(c1ccc(-c2ccccc2)cc1)c1ccc2c(c1)sc1cc(N(c3ccc(-c4ccccc4)cc3)c3ccccc3C)ccc12 ; Cc1cccc(N(c2ccc(-c3ccccc3)cc2)c2ccc3c(c2)sc2cc(N(c4ccc(-c5ccccc5)cc4)c4cccc(C)c4)ccc23)c1 ; Cc1ccc(N(c2ccc(-c3ccccc3)cc2)c2ccc3c(c2)sc2cc(N(c4ccc(C)cc4)c4ccc(-c5ccccc5)cc4)ccc23)cc1] 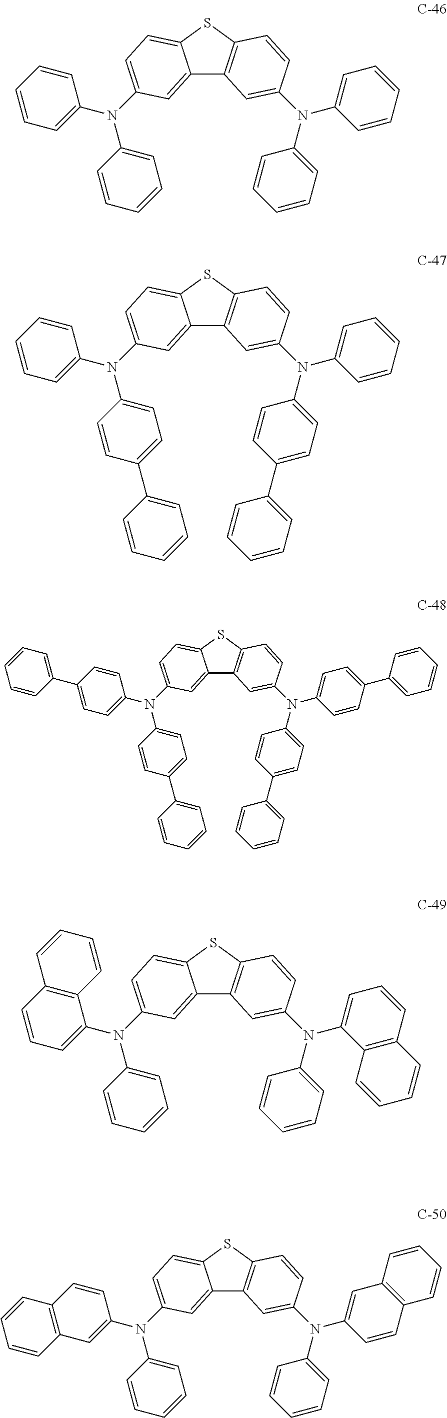 [SMILES: c1ccc(N(c2ccccc2)c2ccc3sc4ccc(N(c5ccccc5)c5ccccc5)cc4c3c2)cc1 ; c1ccc(-c2ccc(N(c3ccccc3)c3ccc4sc5ccc(N(c6ccccc6)c6ccc(-c7ccccc7)cc6)cc5c4c3)cc2)cc1 ; c1ccc(-c2ccc(N(c3ccc(-c4ccccc4)cc3)c3ccc4sc5ccc(N(c6ccc(-c7ccccc7)cc6)c6ccc(-c7ccccc7)cc6)cc5c4c3)cc2)cc1 ; c1ccc(N(c2ccc3sc4ccc(N(c5ccccc5)c5cccc6ccccc56)cc4c3c2)c2cccc3ccccc23)cc1 ; c1ccc(N(c2ccc3ccccc3c2)c2ccc3sc4ccc(N(c5ccccc5)c5ccc6ccccc6c5)cc4c3c2)cc1] 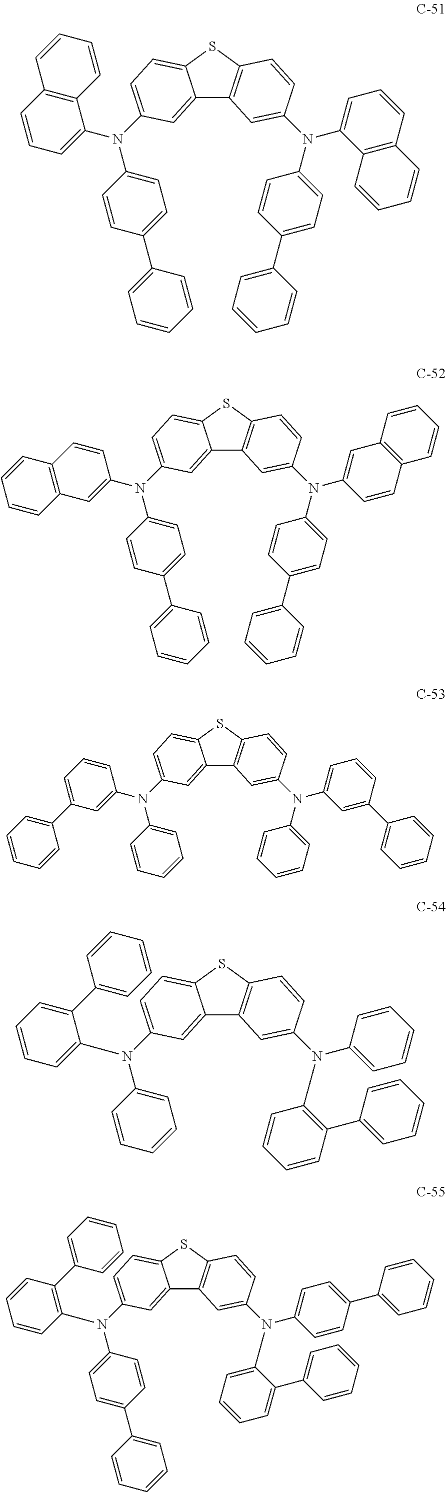 [SMILES: c1ccc(-c2ccc(N(c3ccc4sc5ccc(N(c6ccc(-c7ccccc7)cc6)c6cccc7ccccc67)cc5c4c3)c3cccc4ccccc34)cc2)cc1 ; c1ccc(-c2ccc(N(c3ccc4ccccc4c3)c3ccc4sc5ccc(N(c6ccc(-c7ccccc7)cc6)c6ccc7ccccc7c6)cc5c4c3)cc2)cc1 ; c1ccc(-c2cccc(N(c3ccccc3)c3ccc4sc5ccc(N(c6ccccc6)c6cccc(-c7ccccc7)c6)cc5c4c3)c2)cc1 ; c1ccc(-c2ccccc2N(c2ccccc2)c2ccc3sc4ccc(N(c5ccccc5)c5ccccc5-c5ccccc5)cc4c3c2)cc1 ; c1ccc(-c2ccc(N(c3ccc4sc5ccc(N(c6ccc(-c7ccccc7)cc6)c6ccccc6-c6ccccc6)cc5c4c3)c3ccccc3-c3ccccc3)cc2)cc1] 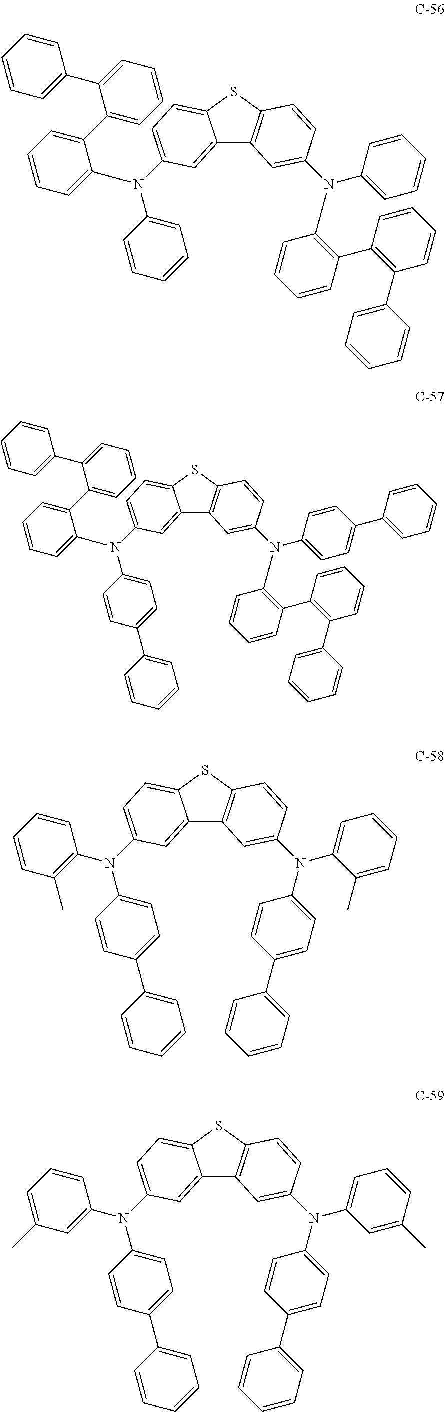 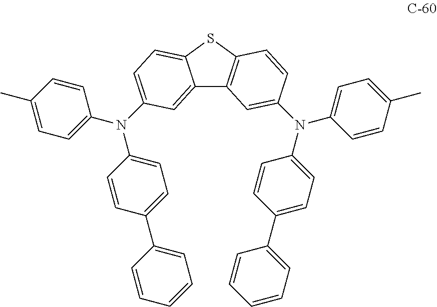 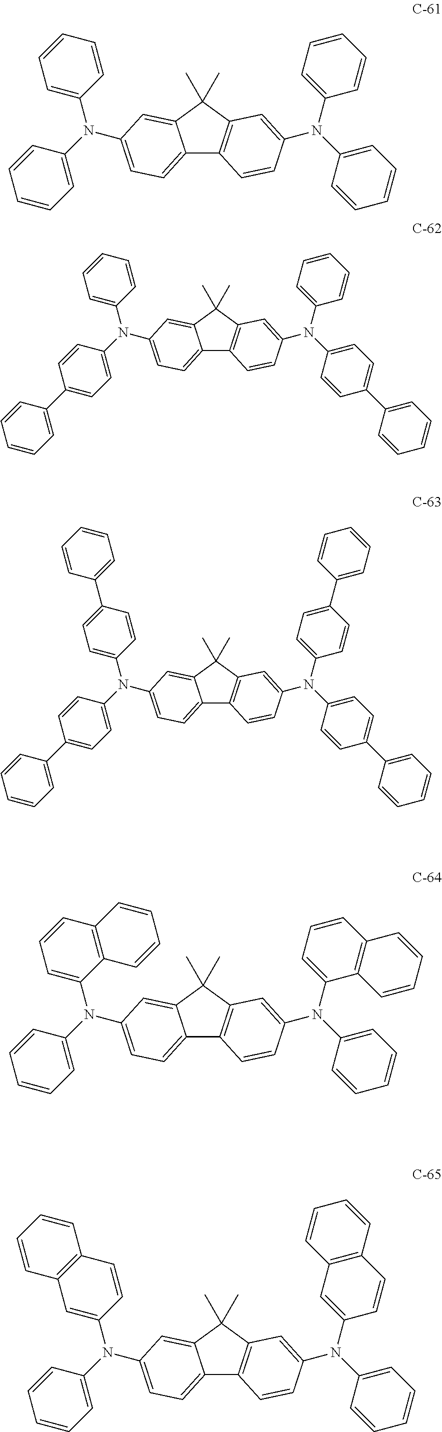 [SMILES: CC1(C)c2cc(N(c3ccccc3)c3ccccc3)ccc2-c2ccc(N(c3ccccc3)c3ccccc3)cc21 ; CC1(C)c2cc(N(c3ccccc3)c3ccc(-c4ccccc4)cc3)ccc2-c2ccc(N(c3ccccc3)c3ccc(-c4ccccc4)cc3)cc21 ; CC1(C)c2cc(N(c3ccc(-c4ccccc4)cc3)c3ccc(-c4ccccc4)cc3)ccc2-c2ccc(N(c3ccc(-c4ccccc4)cc3)c3ccc(-c4ccccc4)cc3)cc21 ; CC1(C)c2cc(N(c3ccccc3)c3cccc4ccccc34)ccc2-c2ccc(N(c3ccccc3)c3cccc4ccccc34)cc21 ; CC1(C)c2cc(N(c3ccccc3)c3ccc4ccccc4c3)ccc2-c2ccc(N(c3ccccc3)c3ccc4ccccc4c3)cc21] 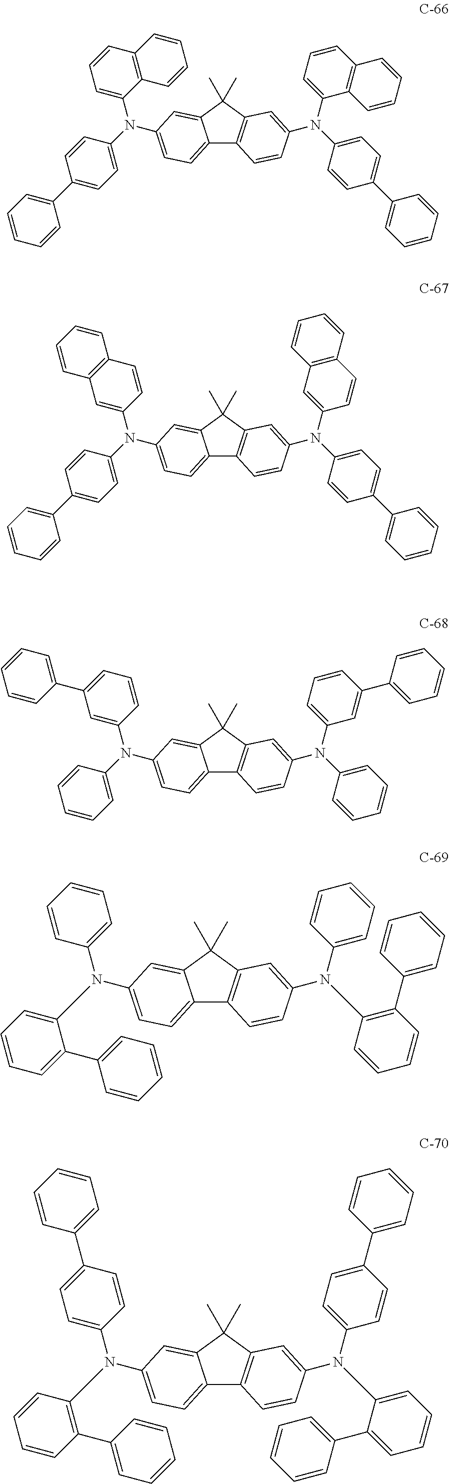 [SMILES: CC1(C)c2cc(N(c3ccc(-c4ccccc4)cc3)c3cccc4ccccc34)ccc2-c2ccc(N(c3ccc(-c4ccccc4)cc3)c3cccc4ccccc34)cc21 ; CC1(C)c2cc(N(c3ccc(-c4ccccc4)cc3)c3ccc4ccccc4c3)ccc2-c2ccc(N(c3ccc(-c4ccccc4)cc3)c3ccc4ccccc4c3)cc21 ; CC1(C)c2cc(N(c3ccccc3)c3cccc(-c4ccccc4)c3)ccc2-c2ccc(N(c3ccccc3)c3cccc(-c4ccccc4)c3)cc21 ; CC1(C)c2cc(N(c3ccccc3)c3ccccc3-c3ccccc3)ccc2-c2ccc(N(c3ccccc3)c3ccccc3-c3ccccc3)cc21 ; CC1(C)c2cc(N(c3ccc(-c4ccccc4)cc3)c3ccccc3-c3ccccc3)ccc2-c2ccc(N(c3ccc(-c4ccccc4)cc3)c3ccccc3-c3ccccc3)cc21] 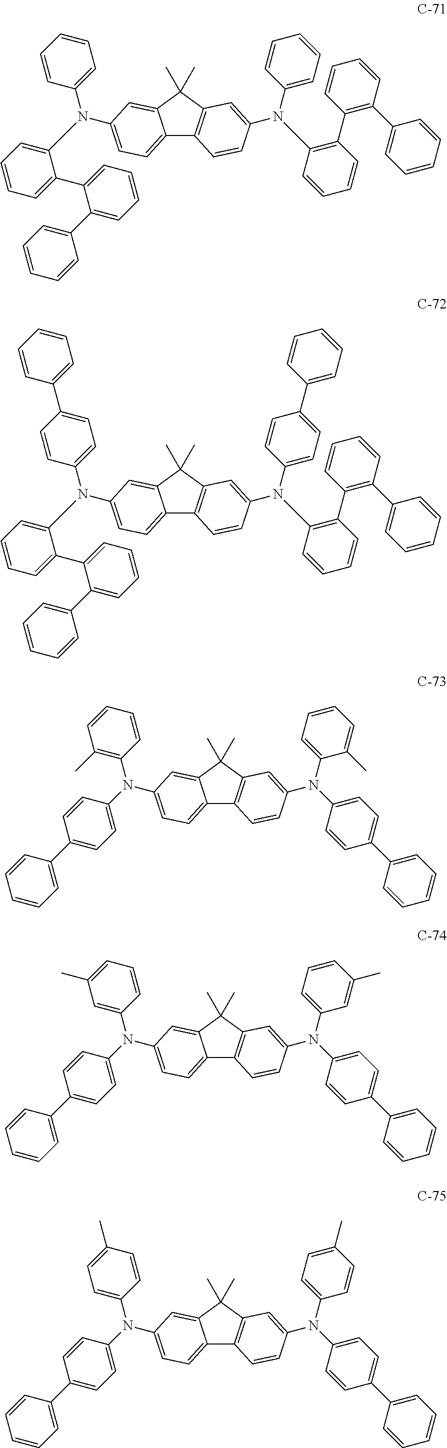 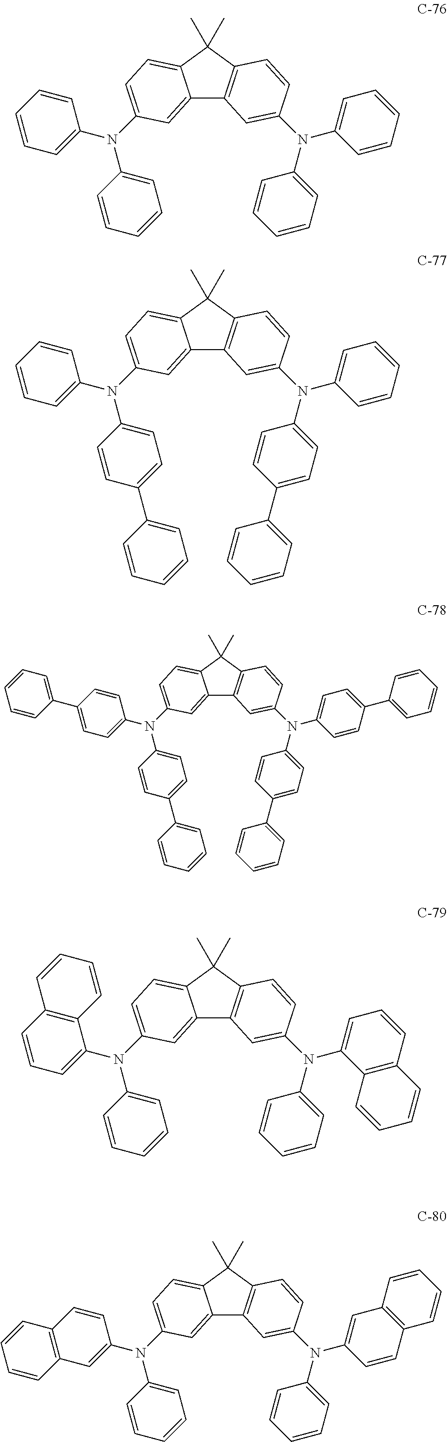 [SMILES: CC1(C)c2ccc(N(c3ccccc3)c3ccccc3)cc2-c2cc(N(c3ccccc3)c3ccccc3)ccc21 ; CC1(C)c2ccc(N(c3ccccc3)c3ccc(-c4ccccc4)cc3)cc2-c2cc(N(c3ccccc3)c3ccc(-c4ccccc4)cc3)ccc21 ; CC1(C)c2ccc(N(c3ccc(-c4ccccc4)cc3)c3ccc(-c4ccccc4)cc3)cc2-c2cc(N(c3ccc(-c4ccccc4)cc3)c3ccc(-c4ccccc4)cc3)ccc21 ; CC1(C)c2ccc(N(c3ccccc3)c3cccc4ccccc34)cc2-c2cc(N(c3ccccc3)c3cccc4ccccc34)ccc21 ; CC1(C)c2ccc(N(c3ccccc3)c3ccc4ccccc4c3)cc2-c2cc(N(c3ccccc3)c3ccc4ccccc4c3)ccc21] 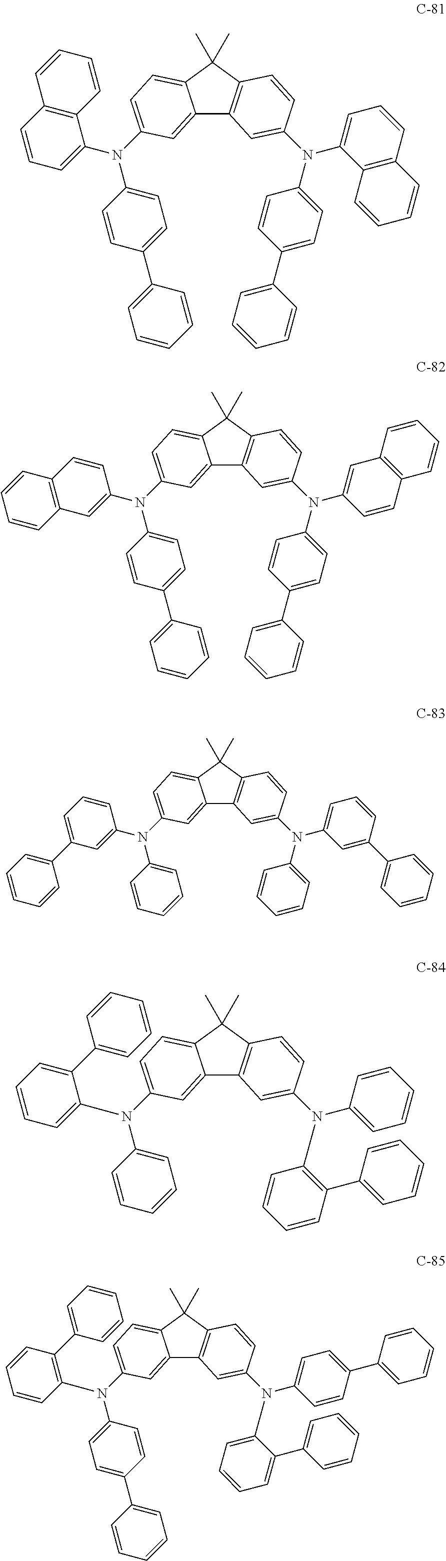 [SMILES: CC1(C)c2ccc(N(c3ccc(-c4ccccc4)cc3)c3cccc4ccccc34)cc2-c2cc(N(c3ccc(-c4ccccc4)cc3)c3cccc4ccccc34)ccc21 ; CC1(C)c2ccc(N(c3ccc(-c4ccccc4)cc3)c3ccc4ccccc4c3)cc2-c2cc(N(c3ccc(-c4ccccc4)cc3)c3ccc4ccccc4c3)ccc21 ; CC1(C)c2ccc(N(c3ccccc3)c3cccc(-c4ccccc4)c3)cc2-c2cc(N(c3ccccc3)c3cccc(-c4ccccc4)c3)ccc21 ; CC1(C)c2ccc(N(c3ccccc3)c3ccccc3-c3ccccc3)cc2-c2cc(N(c3ccccc3)c3ccccc3-c3ccccc3)ccc21 ; CC1(C)c2ccc(N(c3ccc(-c4ccccc4)cc3)c3ccccc3-c3ccccc3)cc2-c2cc(N(c3ccc(-c4ccccc4)cc3)c3ccccc3-c3ccccc3)ccc21] 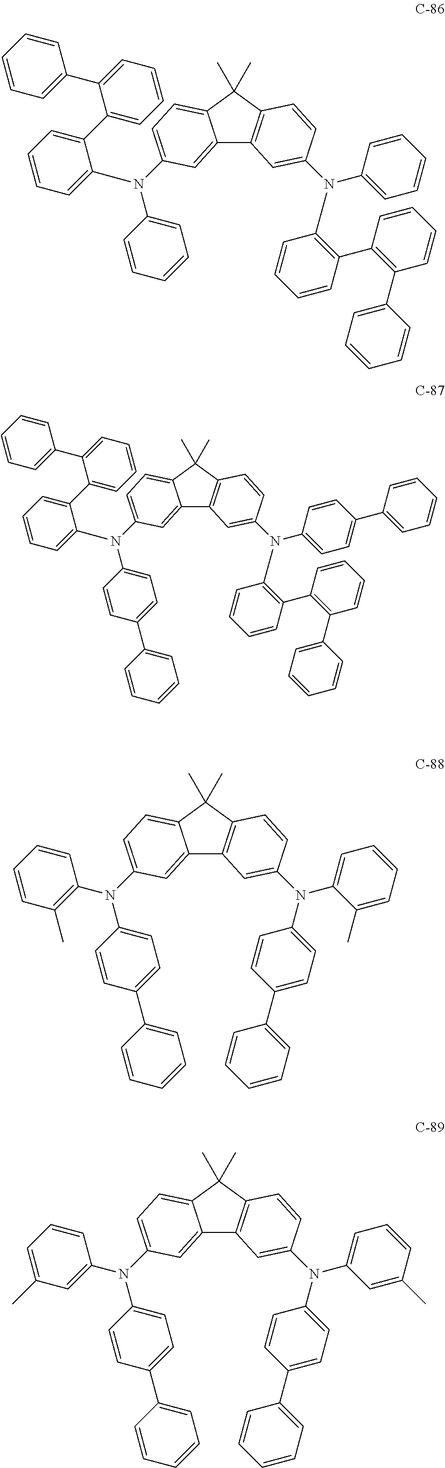 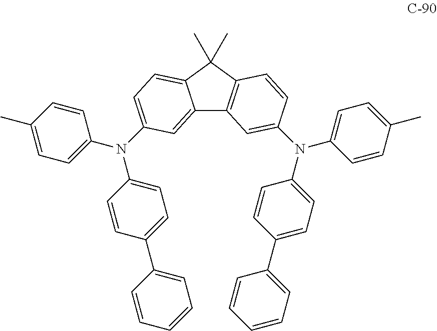 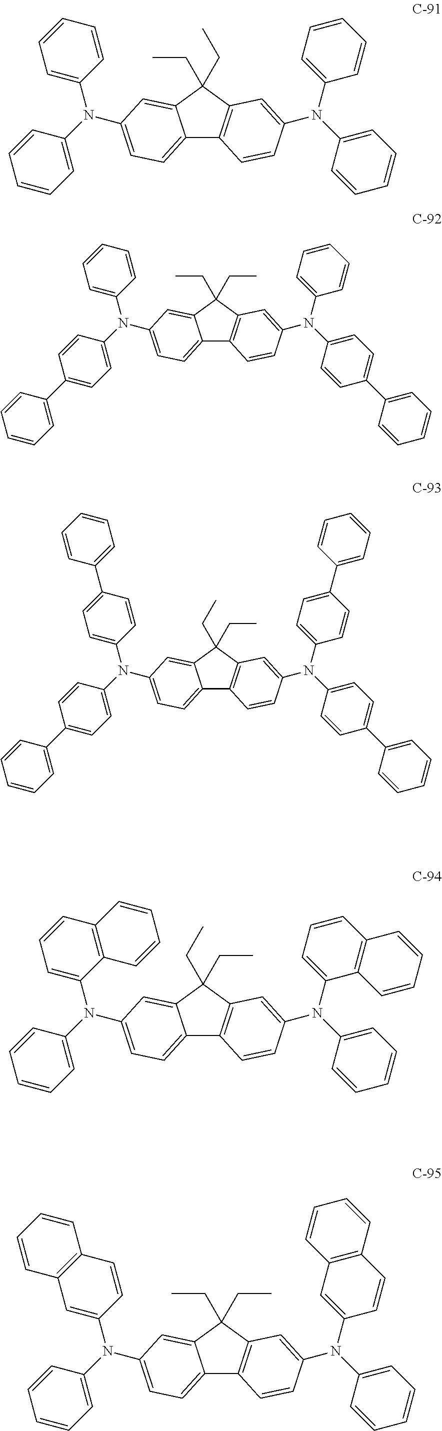 [SMILES: CCC1(CC)c2cc(N(c3ccccc3)c3ccccc3)ccc2-c2ccc(N(c3ccccc3)c3ccccc3)cc21 ; CCC1(CC)c2cc(N(c3ccccc3)c3ccc(-c4ccccc4)cc3)ccc2-c2ccc(N(c3ccccc3)c3ccc(-c4ccccc4)cc3)cc21 ; CCC1(CC)c2cc(N(c3ccc(-c4ccccc4)cc3)c3ccc(-c4ccccc4)cc3)ccc2-c2ccc(N(c3ccc(-c4ccccc4)cc3)c3ccc(-c4ccccc4)cc3)cc21 ; CCC1(CC)c2cc(N(c3ccccc3)c3cccc4ccccc34)ccc2-c2ccc(N(c3ccccc3)c3cccc4ccccc34)cc21 ; CCC1(CC)c2cc(N(c3ccccc3)c3ccc4ccccc4c3)ccc2-c2ccc(N(c3ccccc3)c3ccc4ccccc4c3)cc21] 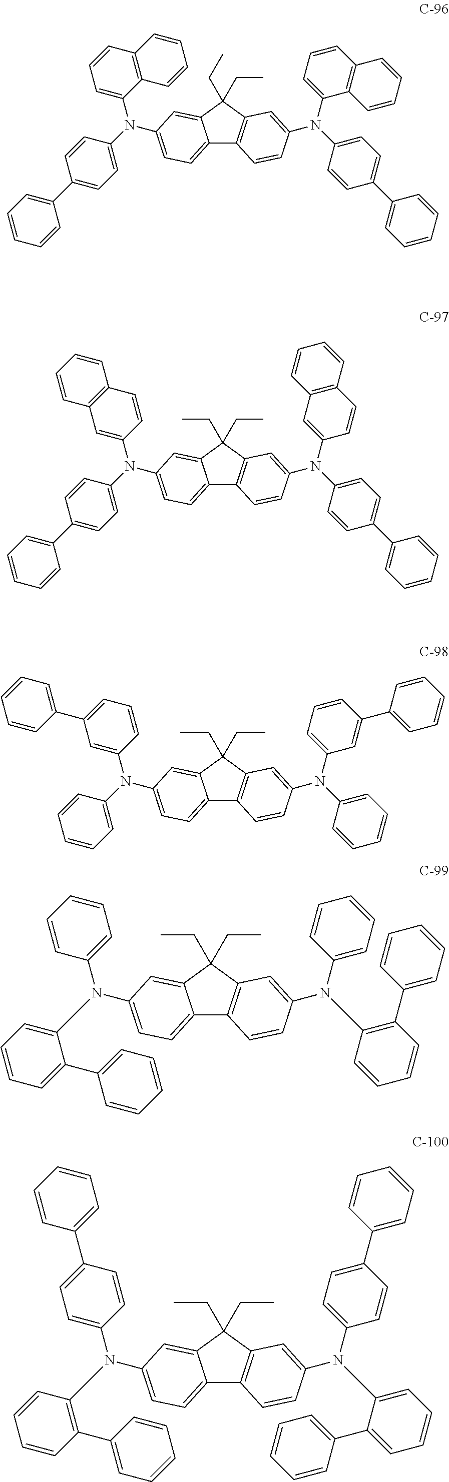 [SMILES: CCC1(CC)c2cc(N(c3ccc(-c4ccccc4)cc3)c3cccc4ccccc34)ccc2-c2ccc(N(c3ccc(-c4ccccc4)cc3)c3cccc4ccccc34)cc21 ; CCC1(CC)c2cc(N(c3ccc(-c4ccccc4)cc3)c3ccc4ccccc4c3)ccc2-c2ccc(N(c3ccc(-c4ccccc4)cc3)c3ccc4ccccc4c3)cc21 ; CCC1(CC)c2cc(N(c3ccccc3)c3cccc(-c4ccccc4)c3)ccc2-c2ccc(N(c3ccccc3)c3cccc(-c4ccccc4)c3)cc21 ; CCC1(CC)c2cc(N(c3ccccc3)c3ccccc3-c3ccccc3)ccc2-c2ccc(N(c3ccccc3)c3ccccc3-c3ccccc3)cc21 ; CCC1(CC)c2cc(N(c3ccc(-c4ccccc4)cc3)c3ccccc3-c3ccccc3)ccc2-c2ccc(N(c3ccc(-c4ccccc4)cc3)c3ccccc3-c3ccccc3)cc21] 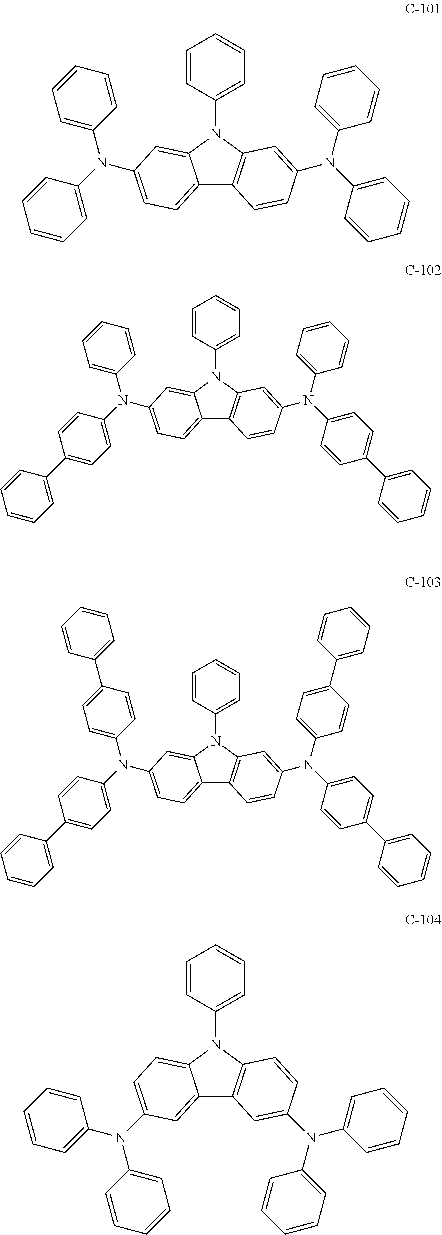 [SMILES: c1ccc(N(c2ccccc2)c2ccc3c4ccc(N(c5ccccc5)c5ccccc5)cc4n(-c4ccccc4)c3c2)cc1 ; c1ccc(-c2ccc(N(c3ccccc3)c3ccc4c5ccc(N(c6ccccc6)c6ccc(-c7ccccc7)cc6)cc5n(-c5ccccc5)c4c3)cc2)cc1 ; c1ccc(-c2ccc(N(c3ccc(-c4ccccc4)cc3)c3ccc4c5ccc(N(c6ccc(-c7ccccc7)cc6)c6ccc(-c7ccccc7)cc6)cc5n(-c5ccccc5)c4c3)cc2)cc1 ; c1ccc(N(c2ccccc2)c2ccc3c(c2)c2cc(N(c4ccccc4)c4ccccc4)ccc2n3-c2ccccc2)cc1] 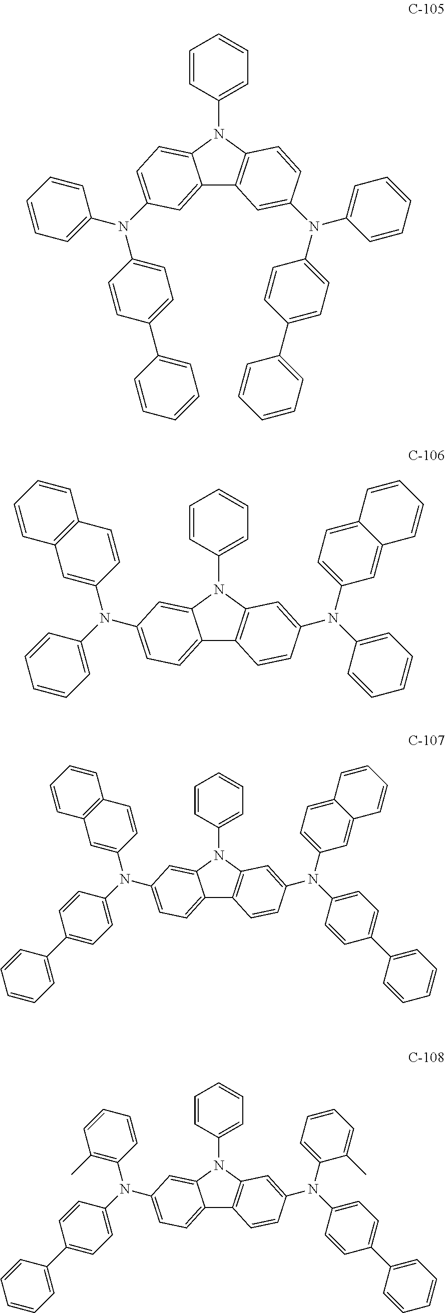 [SMILES: c1ccc(-c2ccc(N(c3ccccc3)c3ccc4c(c3)c3cc(N(c5ccccc5)c5ccc(-c6ccccc6)cc5)ccc3n4-c3ccccc3)cc2)cc1 ; c1ccc(N(c2ccc3ccccc3c2)c2ccc3c4ccc(N(c5ccccc5)c5ccc6ccccc6c5)cc4n(-c4ccccc4)c3c2)cc1 ; c1ccc(-c2ccc(N(c3ccc4ccccc4c3)c3ccc4c5ccc(N(c6ccc(-c7ccccc7)cc6)c6ccc7ccccc7c6)cc5n(-c5ccccc5)c4c3)cc2)cc1 ; Cc1ccccc1N(c1ccc(-c2ccccc2)cc1)c1ccc2c3ccc(N(c4ccc(-c5ccccc5)cc4)c4ccccc4C)cc3n(-c3ccccc3)c2c1] 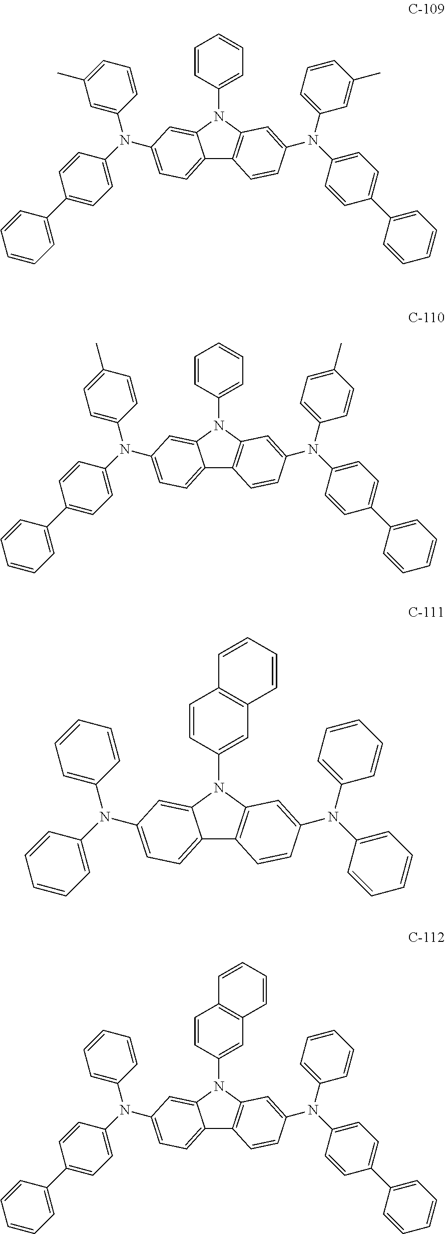 [SMILES: Cc1cccc(N(c2ccc(-c3ccccc3)cc2)c2ccc3c4ccc(N(c5ccc(-c6ccccc6)cc5)c5cccc(C)c5)cc4n(-c4ccccc4)c3c2)c1 ; Cc1ccc(N(c2ccc(-c3ccccc3)cc2)c2ccc3c4ccc(N(c5ccc(C)cc5)c5ccc(-c6ccccc6)cc5)cc4n(-c4ccccc4)c3c2)cc1 ; c1ccc(N(c2ccccc2)c2ccc3c4ccc(N(c5ccccc5)c5ccccc5)cc4n(-c4ccc5ccccc5c4)c3c2)cc1 ; c1ccc(-c2ccc(N(c3ccccc3)c3ccc4c5ccc(N(c6ccccc6)c6ccc(-c7ccccc7)cc6)cc5n(-c5ccc6ccccc6c5)c4c3)cc2)cc1] 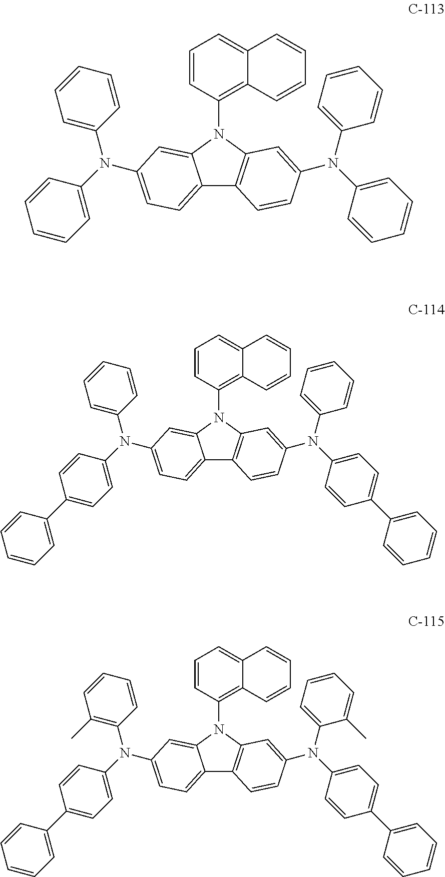 [SMILES: c1ccc(N(c2ccccc2)c2ccc3c4ccc(N(c5ccccc5)c5ccccc5)cc4n(-c4cccc5ccccc45)c3c2)cc1 ; c1ccc(-c2ccc(N(c3ccccc3)c3ccc4c5ccc(N(c6ccccc6)c6ccc(-c7ccccc7)cc6)cc5n(-c5cccc6ccccc56)c4c3)cc2)cc1 ; Cc1ccccc1N(c1ccc(-c2ccccc2)cc1)c1ccc2c3ccc(N(c4ccc(-c5ccccc5)cc4)c4ccccc4C)cc3n(-c3cccc4ccccc34)c2c1] 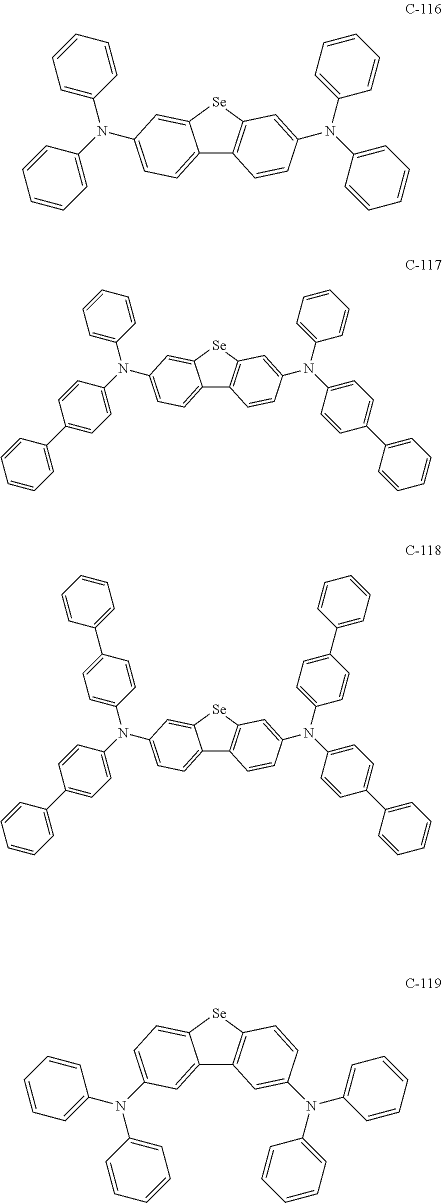 [SMILES: c1ccc(N(c2ccccc2)c2ccc3c(c2)[se]c2cc(N(c4ccccc4)c4ccccc4)ccc23)cc1 ; c1ccc(-c2ccc(N(c3ccccc3)c3ccc4c(c3)[se]c3cc(N(c5ccccc5)c5ccc(-c6ccccc6)cc5)ccc34)cc2)cc1 ; c1ccc(-c2ccc(N(c3ccc(-c4ccccc4)cc3)c3ccc4c(c3)[se]c3cc(N(c5ccc(-c6ccccc6)cc5)c5ccc(-c6ccccc6)cc5)ccc34)cc2)cc1 ; c1ccc(N(c2ccccc2)c2ccc3[se]c4ccc(N(c5ccccc5)c5ccccc5)cc4c3c2)cc1] 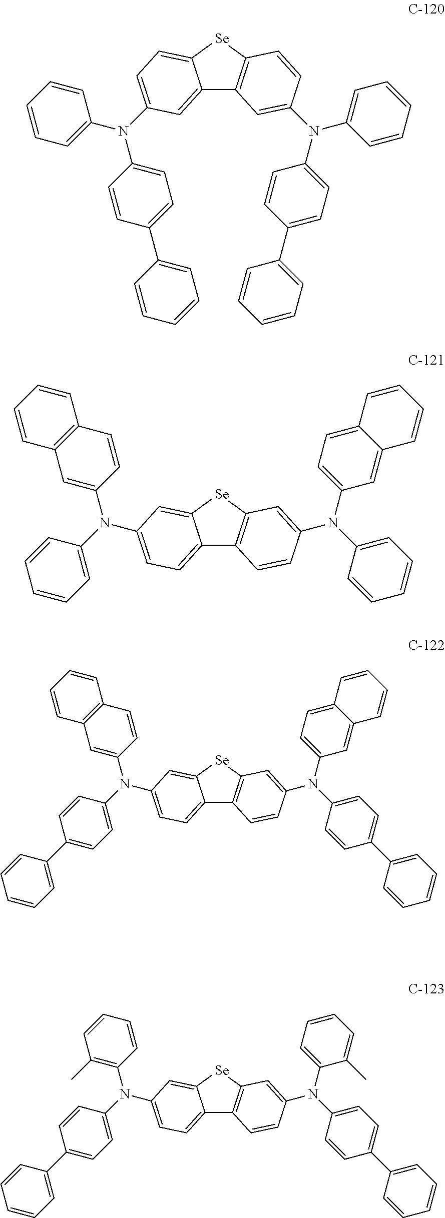 [SMILES: c1ccc(-c2ccc(N(c3ccccc3)c3ccc4[se]c5ccc(N(c6ccccc6)c6ccc(-c7ccccc7)cc6)cc5c4c3)cc2)cc1 ; c1ccc(N(c2ccc3ccccc3c2)c2ccc3c(c2)[se]c2cc(N(c4ccccc4)c4ccc5ccccc5c4)ccc23)cc1 ; c1ccc(-c2ccc(N(c3ccc4ccccc4c3)c3ccc4c(c3)[se]c3cc(N(c5ccc(-c6ccccc6)cc5)c5ccc6ccccc6c5)ccc34)cc2)cc1 ; Cc1ccccc1N(c1ccc(-c2ccccc2)cc1)c1ccc2c(c1)[se]c1cc(N(c3ccc(-c4ccccc4)cc3)c3ccccc3C)ccc12] 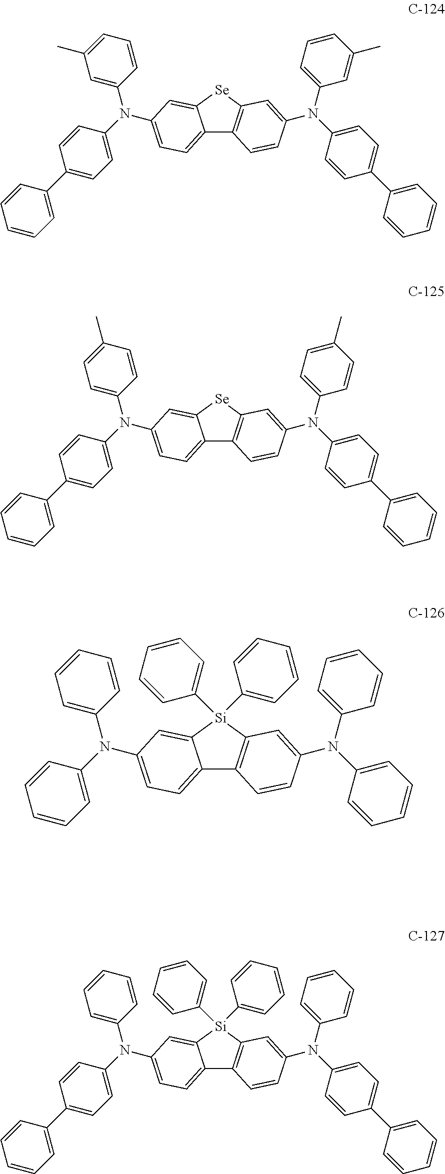 [SMILES: Cc1cccc(N(c2ccc(-c3ccccc3)cc2)c2ccc3c(c2)[se]c2cc(N(c4ccc(-c5ccccc5)cc4)c4cccc(C)c4)ccc23)c1 ; Cc1ccc(N(c2ccc(-c3ccccc3)cc2)c2ccc3c(c2)[se]c2cc(N(c4ccc(C)cc4)c4ccc(-c5ccccc5)cc4)ccc23)cc1 ; c1ccc(N(c2ccccc2)c2ccc3c(c2)[Si](c2ccccc2)(c2ccccc2)c2cc(N(c4ccccc4)c4ccccc4)ccc2-3)cc1 ; c1ccc(-c2ccc(N(c3ccccc3)c3ccc4c(c3)[Si](c3ccccc3)(c3ccccc3)c3cc(N(c5ccccc5)c5ccc(-c6ccccc6)cc5)ccc3-4)cc2)cc1] 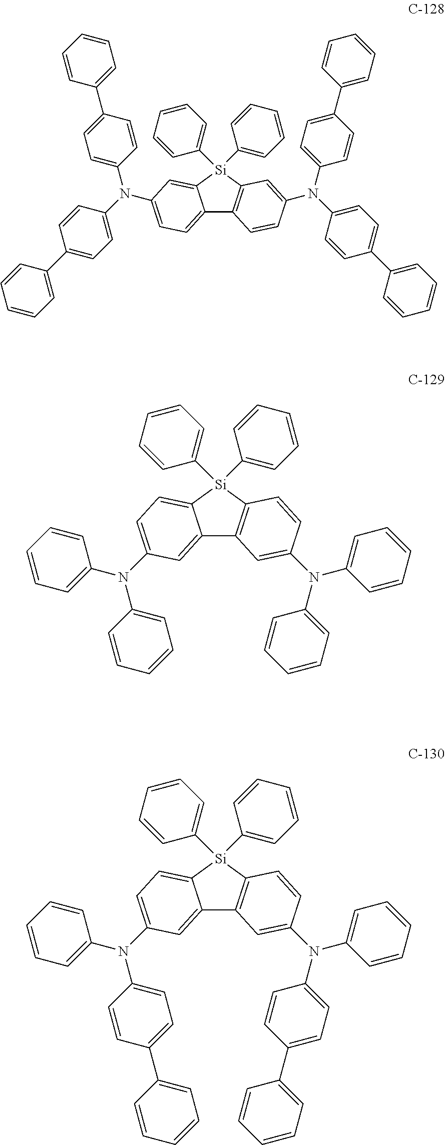 [SMILES: c1ccc(-c2ccc(N(c3ccc(-c4ccccc4)cc3)c3ccc4c(c3)[Si](c3ccccc3)(c3ccccc3)c3cc(N(c5ccc(-c6ccccc6)cc5)c5ccc(-c6ccccc6)cc5)ccc3-4)cc2)cc1 ; c1ccc(N(c2ccccc2)c2ccc3c(c2)-c2cc(N(c4ccccc4)c4ccccc4)ccc2[Si]3(c2ccccc2)c2ccccc2)cc1 ; c1ccc(-c2ccc(N(c3ccccc3)c3ccc4c(c3)-c3cc(N(c5ccccc5)c5ccc(-c6ccccc6)cc5)ccc3[Si]4(c3ccccc3)c3ccccc3)cc2)cc1] 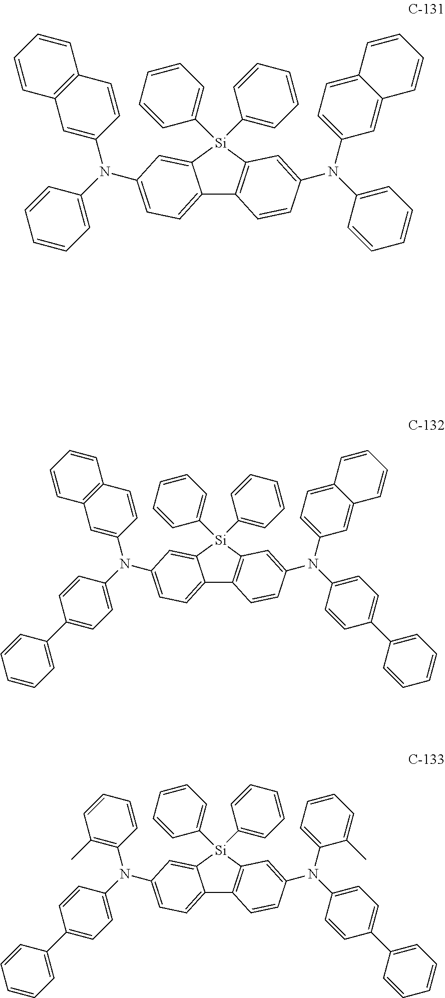 [SMILES: c1ccc(N(c2ccc3c(c2)[Si](c2ccccc2)(c2ccccc2)c2cc(N(c4ccccc4)c4ccc5ccccc5c4)ccc2-3)c2ccc3ccccc3c2)cc1 ; c1ccc(-c2ccc(N(c3ccc4c(c3)[Si](c3ccccc3)(c3ccccc3)c3cc(N(c5ccc(-c6ccccc6)cc5)c5ccc6ccccc6c5)ccc3-4)c3ccc4ccccc4c3)cc2)cc1 ; Cc1ccccc1N(c1ccc(-c2ccccc2)cc1)c1ccc2c(c1)[Si](c1ccccc1)(c1ccccc1)c1cc(N(c3ccc(-c4ccccc4)cc3)c3ccccc3C)ccc1-2] 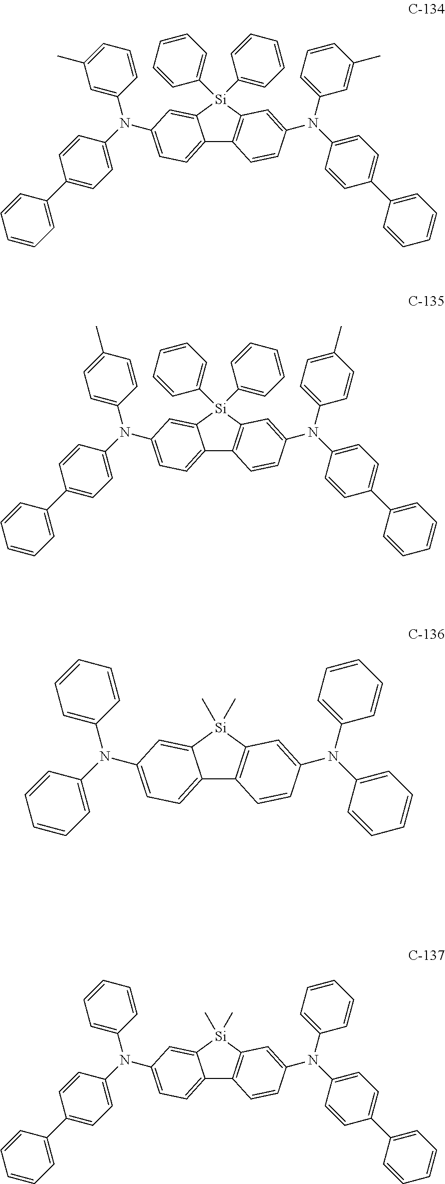 [SMILES: Cc1cccc(N(c2ccc(-c3ccccc3)cc2)c2ccc3c(c2)[Si](c2ccccc2)(c2ccccc2)c2cc(N(c4ccc(-c5ccccc5)cc4)c4cccc(C)c4)ccc2-3)c1 ; Cc1ccc(N(c2ccc(-c3ccccc3)cc2)c2ccc3c(c2)[Si](c2ccccc2)(c2ccccc2)c2cc(N(c4ccc(C)cc4)c4ccc(-c5ccccc5)cc4)ccc2-3)cc1 ; C[Si]1(C)c2cc(N(c3ccccc3)c3ccccc3)ccc2-c2ccc(N(c3ccccc3)c3ccccc3)cc21 ; C[Si]1(C)c2cc(N(c3ccccc3)c3ccc(-c4ccccc4)cc3)ccc2-c2ccc(N(c3ccccc3)c3ccc(-c4ccccc4)cc3)cc21] 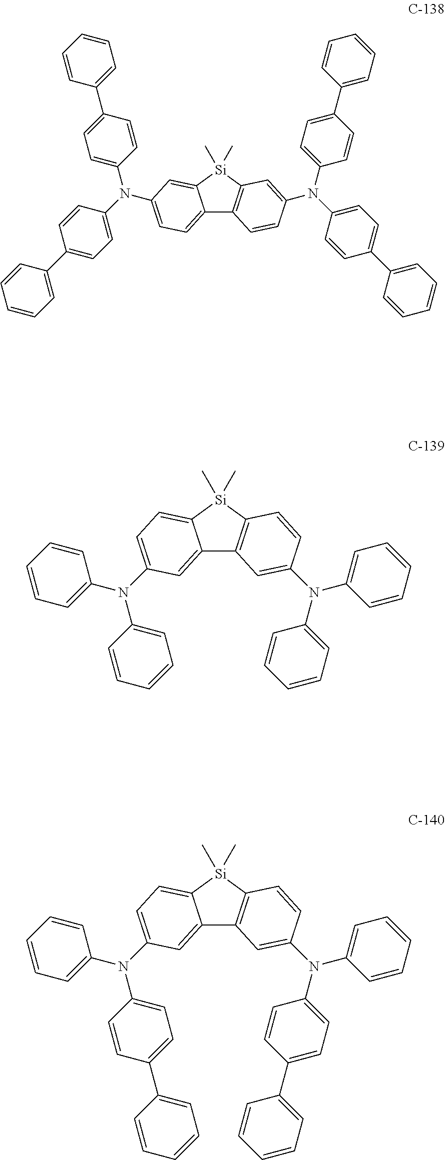 [SMILES: C[Si]1(C)c2cc(N(c3ccc(-c4ccccc4)cc3)c3ccc(-c4ccccc4)cc3)ccc2-c2ccc(N(c3ccc(-c4ccccc4)cc3)c3ccc(-c4ccccc4)cc3)cc21 ; C[Si]1(C)c2ccc(N(c3ccccc3)c3ccccc3)cc2-c2cc(N(c3ccccc3)c3ccccc3)ccc21 ; C[Si]1(C)c2ccc(N(c3ccccc3)c3ccc(-c4ccccc4)cc3)cc2-c2cc(N(c3ccccc3)c3ccc(-c4ccccc4)cc3)ccc21] 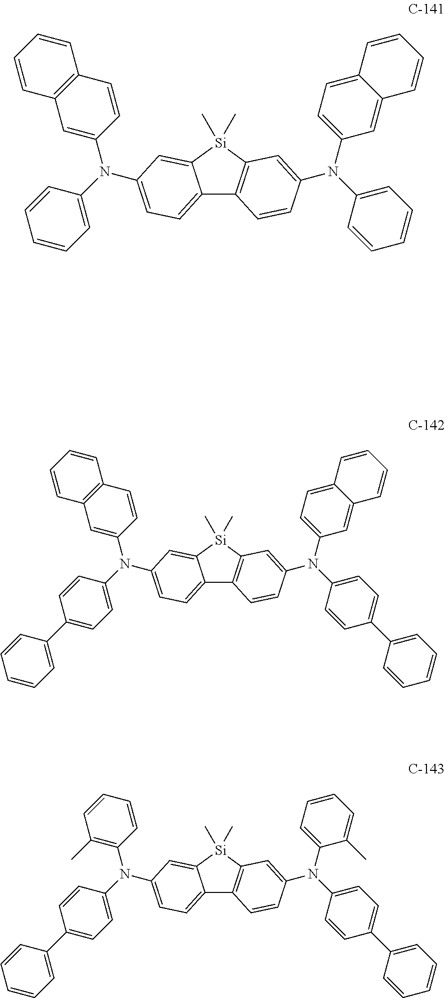 [SMILES: C[Si]1(C)c2cc(N(c3ccccc3)c3ccc4ccccc4c3)ccc2-c2ccc(N(c3ccccc3)c3ccc4ccccc4c3)cc21 ; C[Si]1(C)c2cc(N(c3ccc(-c4ccccc4)cc3)c3ccc4ccccc4c3)ccc2-c2ccc(N(c3ccc(-c4ccccc4)cc3)c3ccc4ccccc4c3)cc21 ; Cc1ccccc1N(c1ccc(-c2ccccc2)cc1)c1ccc2c(c1)[Si](C)(C)c1cc(N(c3ccc(-c4ccccc4)cc3)c3ccccc3C)ccc1-2] 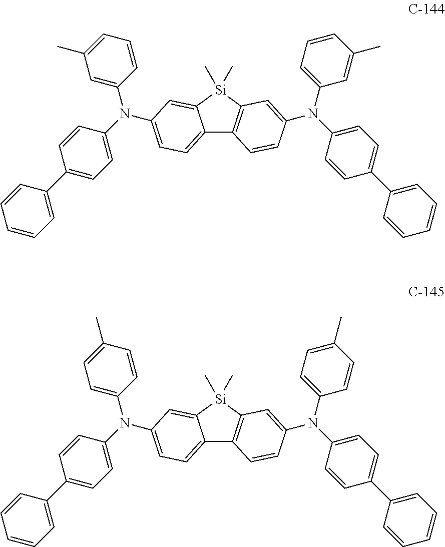 [SMILES: Cc1cccc(N(c2ccc(-c3ccccc3)cc2)c2ccc3c(c2)[Si](C)(C)c2cc(N(c4ccc(-c5ccccc5)cc4)c4cccc(C)c4)ccc2-3)c1 ; Cc1ccc(N(c2ccc(-c3ccccc3)cc2)c2ccc3c(c2)[Si](C)(C)c2cc(N(c4ccc(C)cc4)c4ccc(-c5ccccc5)cc4)ccc2-3)cc1] 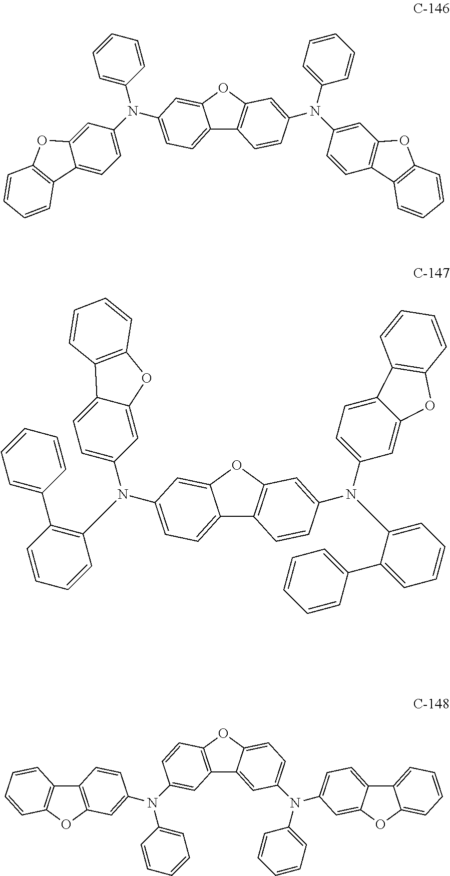 [SMILES: c1ccc(N(c2ccc3c(c2)oc2ccccc23)c2ccc3c(c2)oc2cc(N(c4ccccc4)c4ccc5c(c4)oc4ccccc45)ccc23)cc1 ; c1ccc(-c2ccccc2N(c2ccc3c(c2)oc2ccccc23)c2ccc3c(c2)oc2cc(N(c4ccc5c(c4)oc4ccccc45)c4ccccc4-c4ccccc4)ccc23)cc1 ; c1ccc(N(c2ccc3c(c2)oc2ccccc23)c2ccc3oc4ccc(N(c5ccccc5)c5ccc6c(c5)oc5ccccc56)cc4c3c2)cc1] 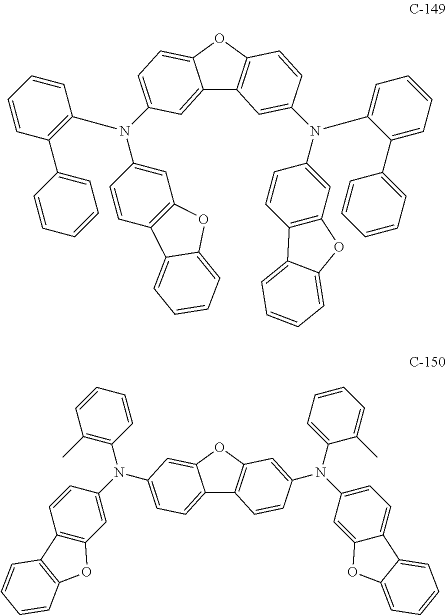 [SMILES: c1ccc(-c2ccccc2N(c2ccc3c(c2)oc2ccccc23)c2ccc3oc4ccc(N(c5ccc6c(c5)oc5ccccc56)c5ccccc5-c5ccccc5)cc4c3c2)cc1 ; Cc1ccccc1N(c1ccc2c(c1)oc1ccccc12)c1ccc2c(c1)oc1cc(N(c3ccc4c(c3)oc3ccccc34)c3ccccc3C)ccc12] 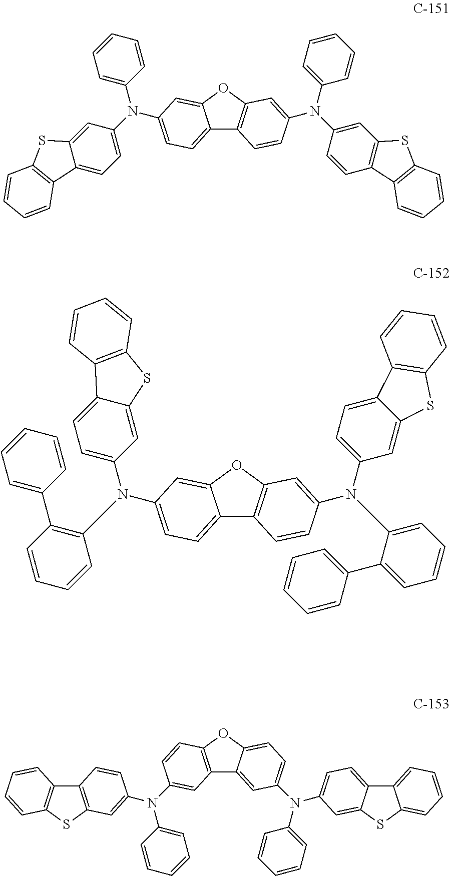 [SMILES: c1ccc(N(c2ccc3c(c2)oc2cc(N(c4ccccc4)c4ccc5c(c4)sc4ccccc45)ccc23)c2ccc3c(c2)sc2ccccc23)cc1 ; c1ccc(-c2ccccc2N(c2ccc3c(c2)oc2cc(N(c4ccc5c(c4)sc4ccccc45)c4ccccc4-c4ccccc4)ccc23)c2ccc3c(c2)sc2ccccc23)cc1 ; c1ccc(N(c2ccc3c(c2)sc2ccccc23)c2ccc3oc4ccc(N(c5ccccc5)c5ccc6c(c5)sc5ccccc56)cc4c3c2)cc1] 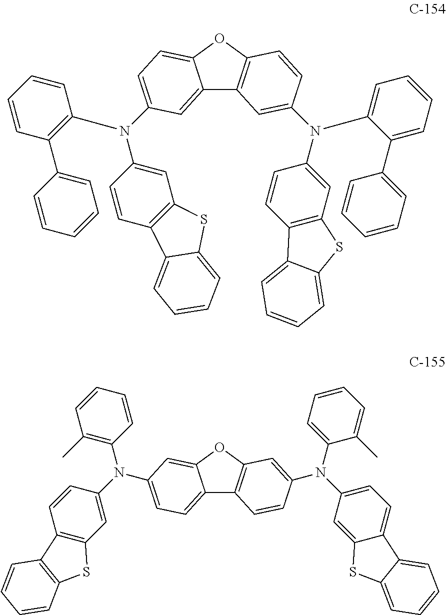 [SMILES: c1ccc(-c2ccccc2N(c2ccc3c(c2)sc2ccccc23)c2ccc3oc4ccc(N(c5ccc6c(c5)sc5ccccc56)c5ccccc5-c5ccccc5)cc4c3c2)cc1 ; Cc1ccccc1N(c1ccc2c(c1)oc1cc(N(c3ccc4c(c3)sc3ccccc34)c3ccccc3C)ccc12)c1ccc2c(c1)sc1ccccc12] 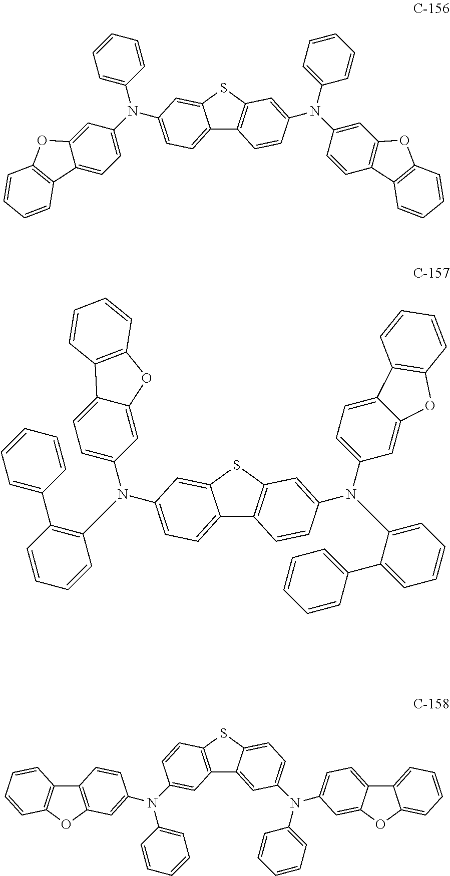 [SMILES: c1ccc(N(c2ccc3c(c2)oc2ccccc23)c2ccc3c(c2)sc2cc(N(c4ccccc4)c4ccc5c(c4)oc4ccccc45)ccc23)cc1 ; c1ccc(-c2ccccc2N(c2ccc3c(c2)oc2ccccc23)c2ccc3c(c2)sc2cc(N(c4ccc5c(c4)oc4ccccc45)c4ccccc4-c4ccccc4)ccc23)cc1 ; c1ccc(N(c2ccc3c(c2)oc2ccccc23)c2ccc3sc4ccc(N(c5ccccc5)c5ccc6c(c5)oc5ccccc56)cc4c3c2)cc1] 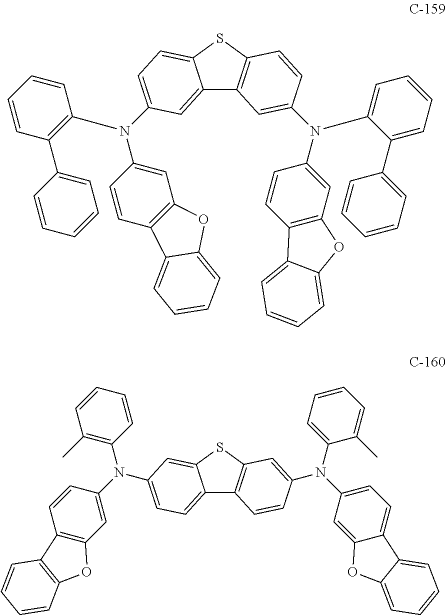 [SMILES: c1ccc(-c2ccccc2N(c2ccc3c(c2)oc2ccccc23)c2ccc3sc4ccc(N(c5ccc6c(c5)oc5ccccc56)c5ccccc5-c5ccccc5)cc4c3c2)cc1 ; Cc1ccccc1N(c1ccc2c(c1)oc1ccccc12)c1ccc2c(c1)sc1cc(N(c3ccc4c(c3)oc3ccccc34)c3ccccc3C)ccc12] 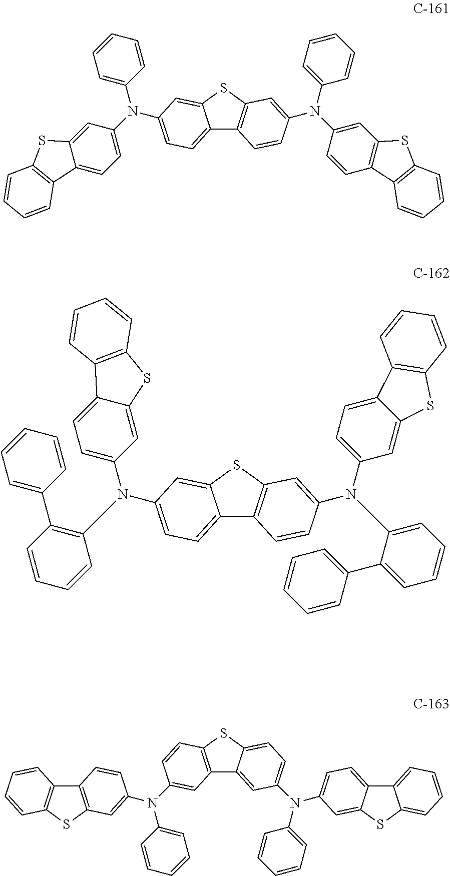 [SMILES: c1ccc(N(c2ccc3c(c2)sc2ccccc23)c2ccc3c(c2)sc2cc(N(c4ccccc4)c4ccc5c(c4)sc4ccccc45)ccc23)cc1 ; c1ccc(-c2ccccc2N(c2ccc3c(c2)sc2ccccc23)c2ccc3c(c2)sc2cc(N(c4ccc5c(c4)sc4ccccc45)c4ccccc4-c4ccccc4)ccc23)cc1 ; c1ccc(N(c2ccc3c(c2)sc2ccccc23)c2ccc3sc4ccc(N(c5ccccc5)c5ccc6c(c5)sc5ccccc56)cc4c3c2)cc1] 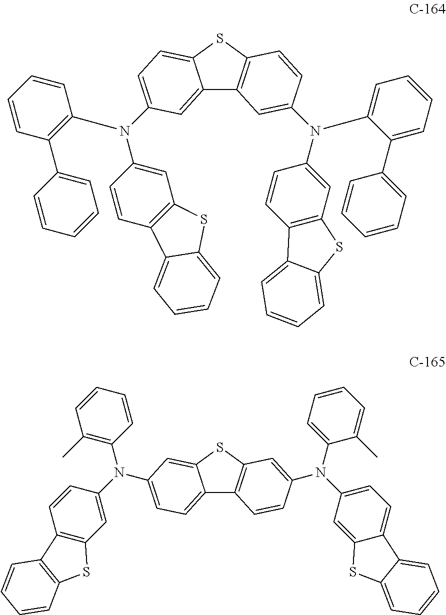 [SMILES: c1ccc(-c2ccccc2N(c2ccc3c(c2)sc2ccccc23)c2ccc3sc4ccc(N(c5ccc6c(c5)sc5ccccc56)c5ccccc5-c5ccccc5)cc4c3c2)cc1 ; Cc1ccccc1N(c1ccc2c(c1)sc1ccccc12)c1ccc2c(c1)sc1cc(N(c3ccc4c(c3)sc3ccccc34)c3ccccc3C)ccc12] 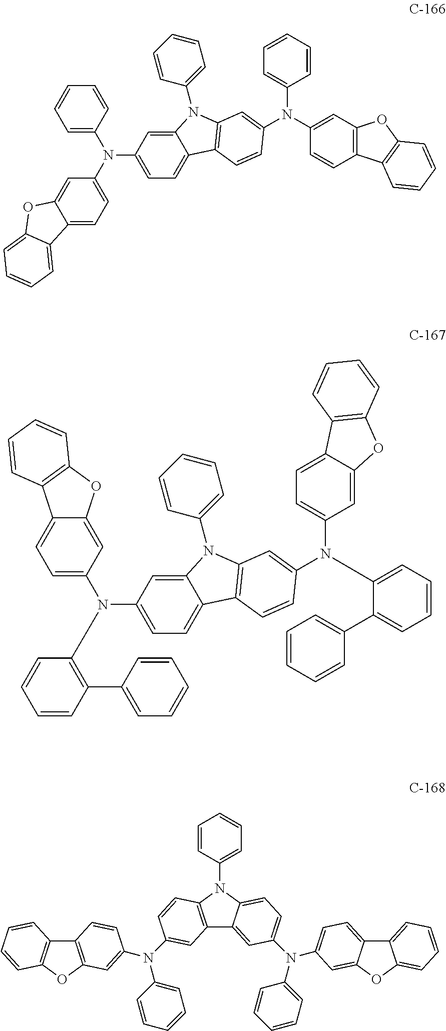 [SMILES: c1ccc(N(c2ccc3c(c2)oc2ccccc23)c2ccc3c4ccc(N(c5ccccc5)c5ccc6c(c5)oc5ccccc56)cc4n(-c4ccccc4)c3c2)cc1 ; c1ccc(-c2ccccc2N(c2ccc3c(c2)oc2ccccc23)c2ccc3c4ccc(N(c5ccc6c(c5)oc5ccccc56)c5ccccc5-c5ccccc5)cc4n(-c4ccccc4)c3c2)cc1 ; c1ccc(N(c2ccc3c(c2)oc2ccccc23)c2ccc3c(c2)c2cc(N(c4ccccc4)c4ccc5c(c4)oc4ccccc45)ccc2n3-c2ccccc2)cc1] 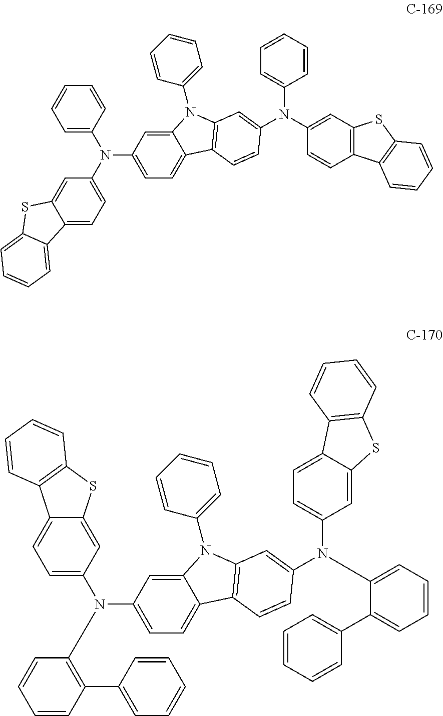 [SMILES: c1ccc(N(c2ccc3c(c2)sc2ccccc23)c2ccc3c4ccc(N(c5ccccc5)c5ccc6c(c5)sc5ccccc56)cc4n(-c4ccccc4)c3c2)cc1 ; c1ccc(-c2ccccc2N(c2ccc3c(c2)sc2ccccc23)c2ccc3c4ccc(N(c5ccc6c(c5)sc5ccccc56)c5ccccc5-c5ccccc5)cc4n(-c4ccccc4)c3c2)cc1] 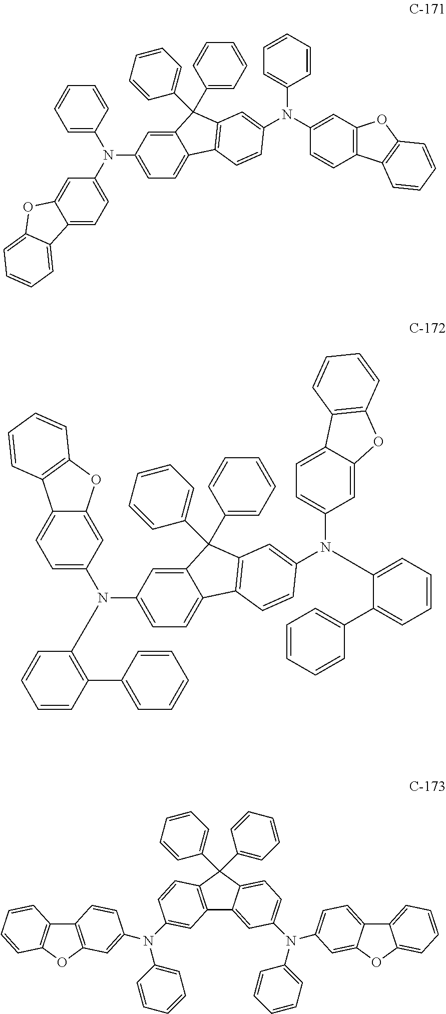 [SMILES: c1ccc(N(c2ccc3c(c2)C(c2ccccc2)(c2ccccc2)c2cc(N(c4ccccc4)c4ccc5c(c4)oc4ccccc45)ccc2-3)c2ccc3c(c2)oc2ccccc23)cc1 ; c1ccc(-c2ccccc2N(c2ccc3c(c2)C(c2ccccc2)(c2ccccc2)c2cc(N(c4ccc5c(c4)oc4ccccc45)c4ccccc4-c4ccccc4)ccc2-3)c2ccc3c(c2)oc2ccccc23)cc1 ; c1ccc(N(c2ccc3c(c2)-c2cc(N(c4ccccc4)c4ccc5c(c4)oc4ccccc45)ccc2C3(c2ccccc2)c2ccccc2)c2ccc3c(c2)oc2ccccc23)cc1] 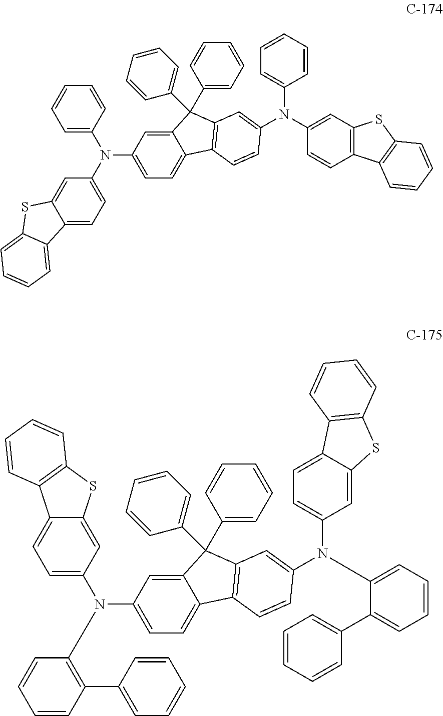 [SMILES: c1ccc(N(c2ccc3c(c2)C(c2ccccc2)(c2ccccc2)c2cc(N(c4ccccc4)c4ccc5c(c4)sc4ccccc45)ccc2-3)c2ccc3c(c2)sc2ccccc23)cc1 ; c1ccc(-c2ccccc2N(c2ccc3c(c2)C(c2ccccc2)(c2ccccc2)c2cc(N(c4ccc5c(c4)sc4ccccc45)c4ccccc4-c4ccccc4)ccc2-3)c2ccc3c(c2)sc2ccccc23)cc1] 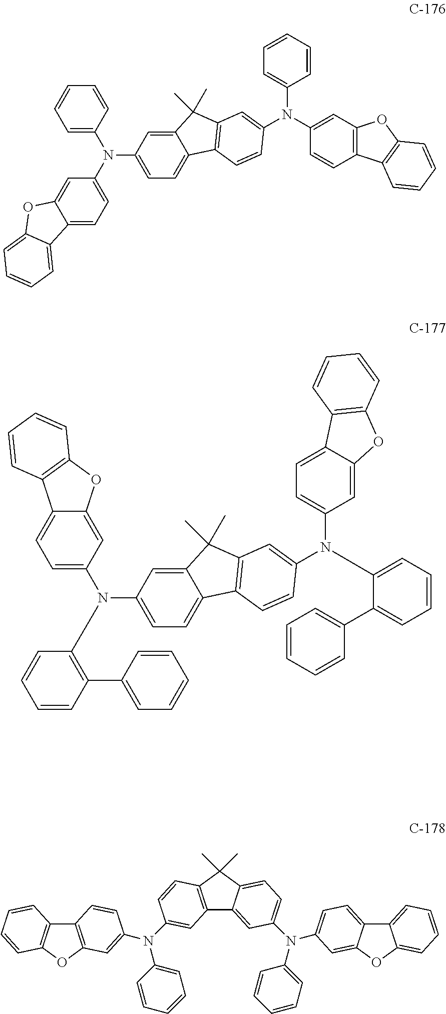 [SMILES: CC1(C)c2cc(N(c3ccccc3)c3ccc4c(c3)oc3ccccc34)ccc2-c2ccc(N(c3ccccc3)c3ccc4c(c3)oc3ccccc34)cc21 ; CC1(C)c2cc(N(c3ccc4c(c3)oc3ccccc34)c3ccccc3-c3ccccc3)ccc2-c2ccc(N(c3ccc4c(c3)oc3ccccc34)c3ccccc3-c3ccccc3)cc21 ; CC1(C)c2ccc(N(c3ccccc3)c3ccc4c(c3)oc3ccccc34)cc2-c2cc(N(c3ccccc3)c3ccc4c(c3)oc3ccccc34)ccc21] 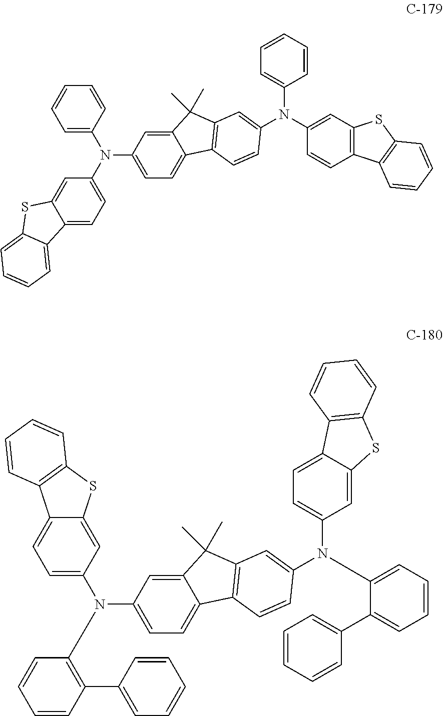 [SMILES: CC1(C)c2cc(N(c3ccccc3)c3ccc4c(c3)sc3ccccc34)ccc2-c2ccc(N(c3ccccc3)c3ccc4c(c3)sc3ccccc34)cc21 ; CC1(C)c2cc(N(c3ccc4c(c3)sc3ccccc34)c3ccccc3-c3ccccc3)ccc2-c2ccc(N(c3ccc4c(c3)sc3ccccc34)c3ccccc3-c3ccccc3)cc21] 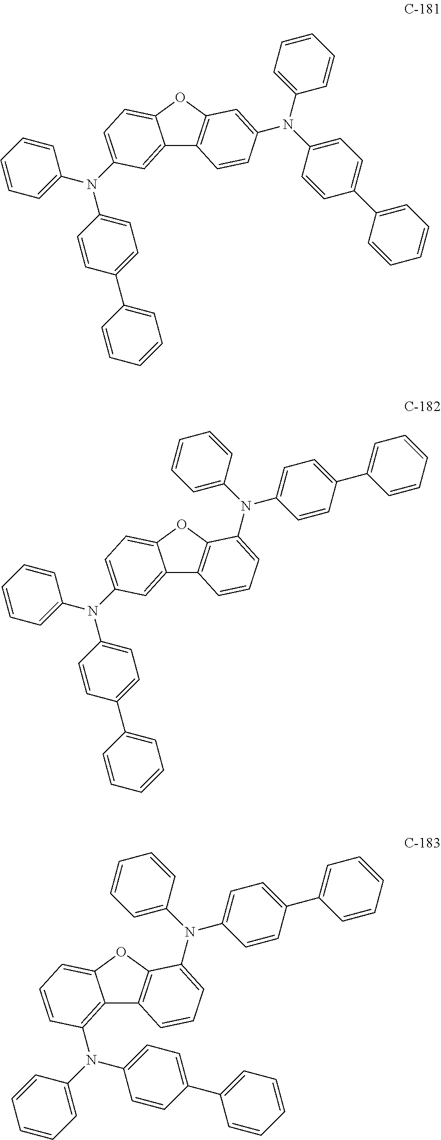 [SMILES: c1ccc(-c2ccc(N(c3ccccc3)c3ccc4c(c3)oc3ccc(N(c5ccccc5)c5ccc(-c6ccccc6)cc5)cc34)cc2)cc1 ; c1ccc(-c2ccc(N(c3ccccc3)c3ccc4oc5c(N(c6ccccc6)c6ccc(-c7ccccc7)cc6)cccc5c4c3)cc2)cc1 ; c1ccc(-c2ccc(N(c3ccccc3)c3cccc4c3oc3cccc(N(c5ccccc5)c5ccc(-c6ccccc6)cc5)c34)cc2)cc1] 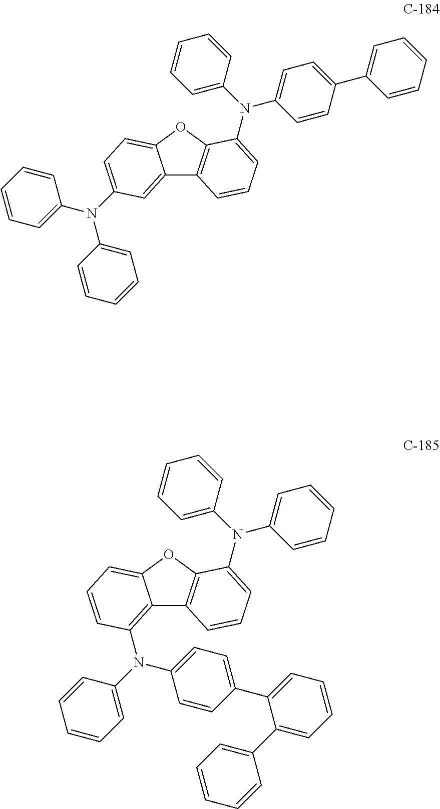 [SMILES: c1ccc(-c2ccc(N(c3ccccc3)c3cccc4c3oc3ccc(N(c5ccccc5)c5ccccc5)cc34)cc2)cc1 ; c1ccc(-c2ccccc2-c2ccc(N(c3ccccc3)c3cccc4oc5c(N(c6ccccc6)c6ccccc6)cccc5c34)cc2)cc1] 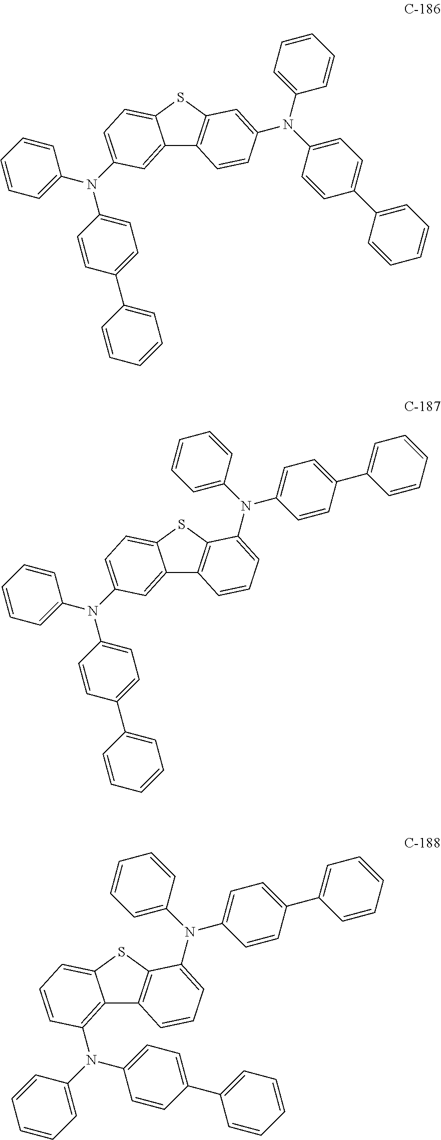 [SMILES: c1ccc(-c2ccc(N(c3ccccc3)c3ccc4c(c3)sc3ccc(N(c5ccccc5)c5ccc(-c6ccccc6)cc5)cc34)cc2)cc1 ; c1ccc(-c2ccc(N(c3ccccc3)c3ccc4sc5c(N(c6ccccc6)c6ccc(-c7ccccc7)cc6)cccc5c4c3)cc2)cc1 ; c1ccc(-c2ccc(N(c3ccccc3)c3cccc4c3sc3cccc(N(c5ccccc5)c5ccc(-c6ccccc6)cc5)c34)cc2)cc1] 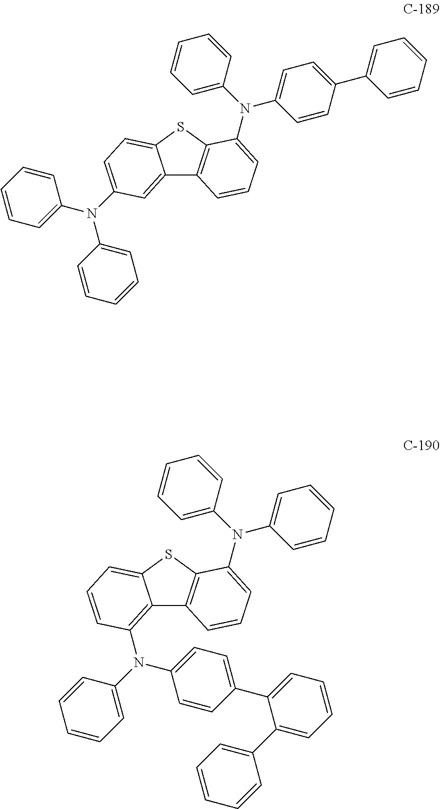 [SMILES: c1ccc(-c2ccc(N(c3ccccc3)c3cccc4c3sc3ccc(N(c5ccccc5)c5ccccc5)cc34)cc2)cc1 ; c1ccc(-c2ccccc2-c2ccc(N(c3ccccc3)c3cccc4sc5c(N(c6ccccc6)c6ccccc6)cccc5c34)cc2)cc1] 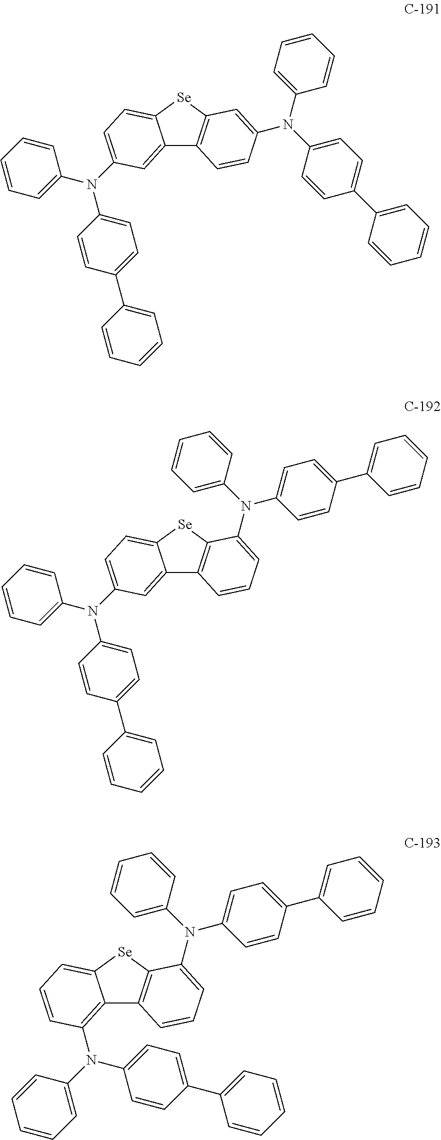 [SMILES: c1ccc(-c2ccc(N(c3ccccc3)c3ccc4c(c3)[se]c3ccc(N(c5ccccc5)c5ccc(-c6ccccc6)cc5)cc34)cc2)cc1 ; c1ccc(-c2ccc(N(c3ccccc3)c3ccc4[se]c5c(N(c6ccccc6)c6ccc(-c7ccccc7)cc6)cccc5c4c3)cc2)cc1 ; c1ccc(-c2ccc(N(c3ccccc3)c3cccc4c3[se]c3cccc(N(c5ccccc5)c5ccc(-c6ccccc6)cc5)c34)cc2)cc1] 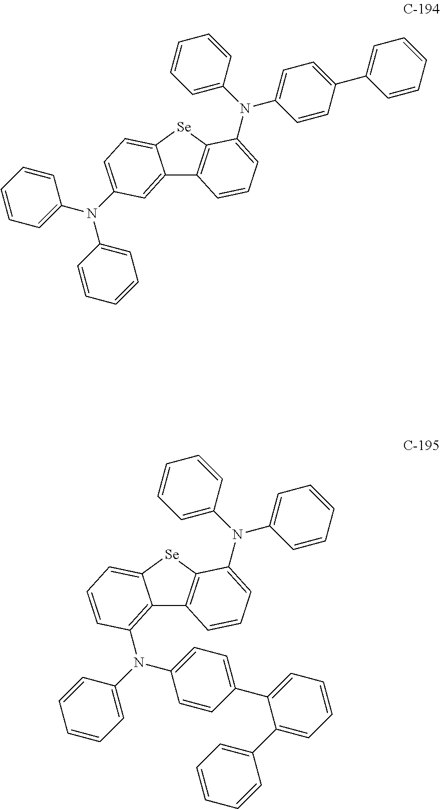 [SMILES: c1ccc(-c2ccc(N(c3ccccc3)c3cccc4c3[se]c3ccc(N(c5ccccc5)c5ccccc5)cc34)cc2)cc1 ; c1ccc(-c2ccccc2-c2ccc(N(c3ccccc3)c3cccc4[se]c5c(N(c6ccccc6)c6ccccc6)cccc5c34)cc2)cc1] 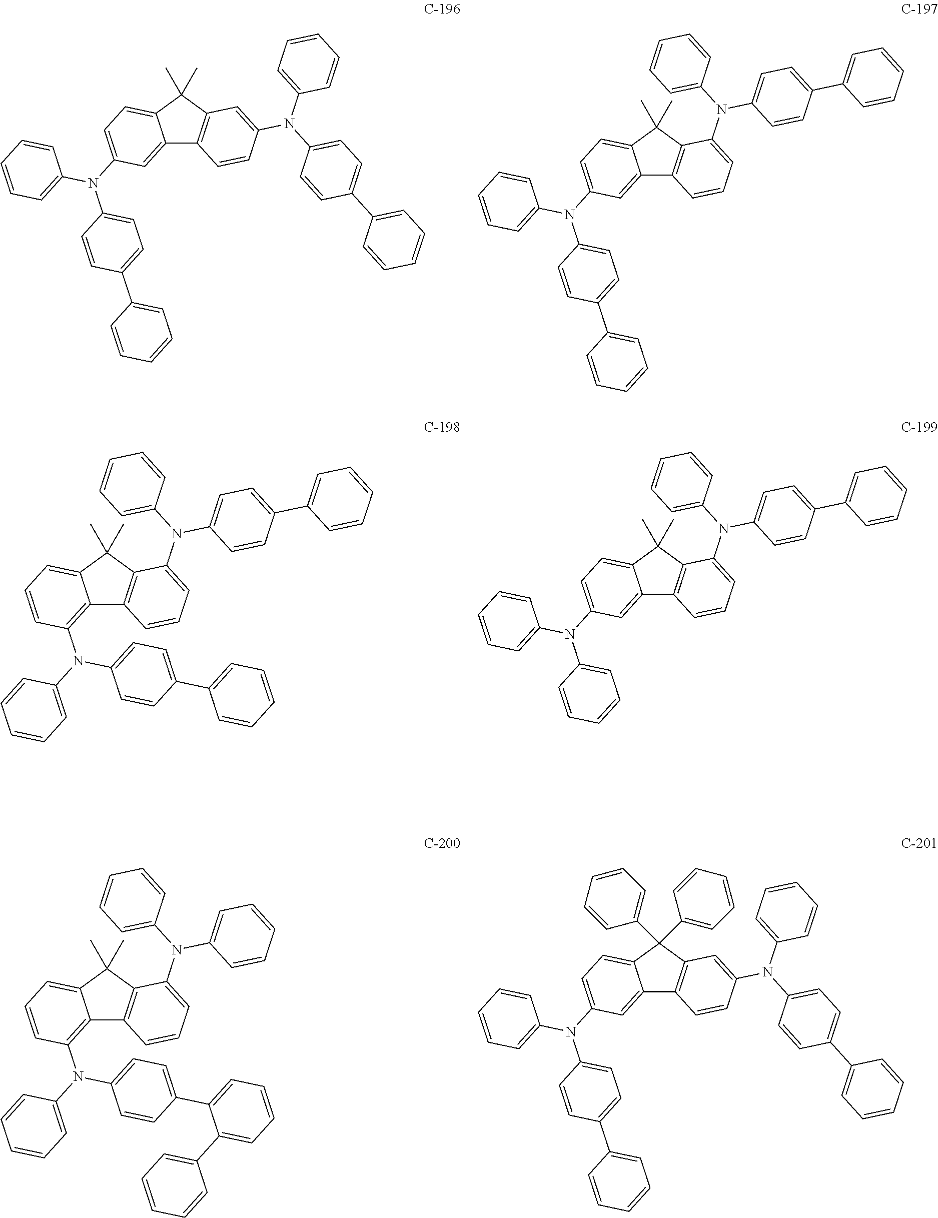 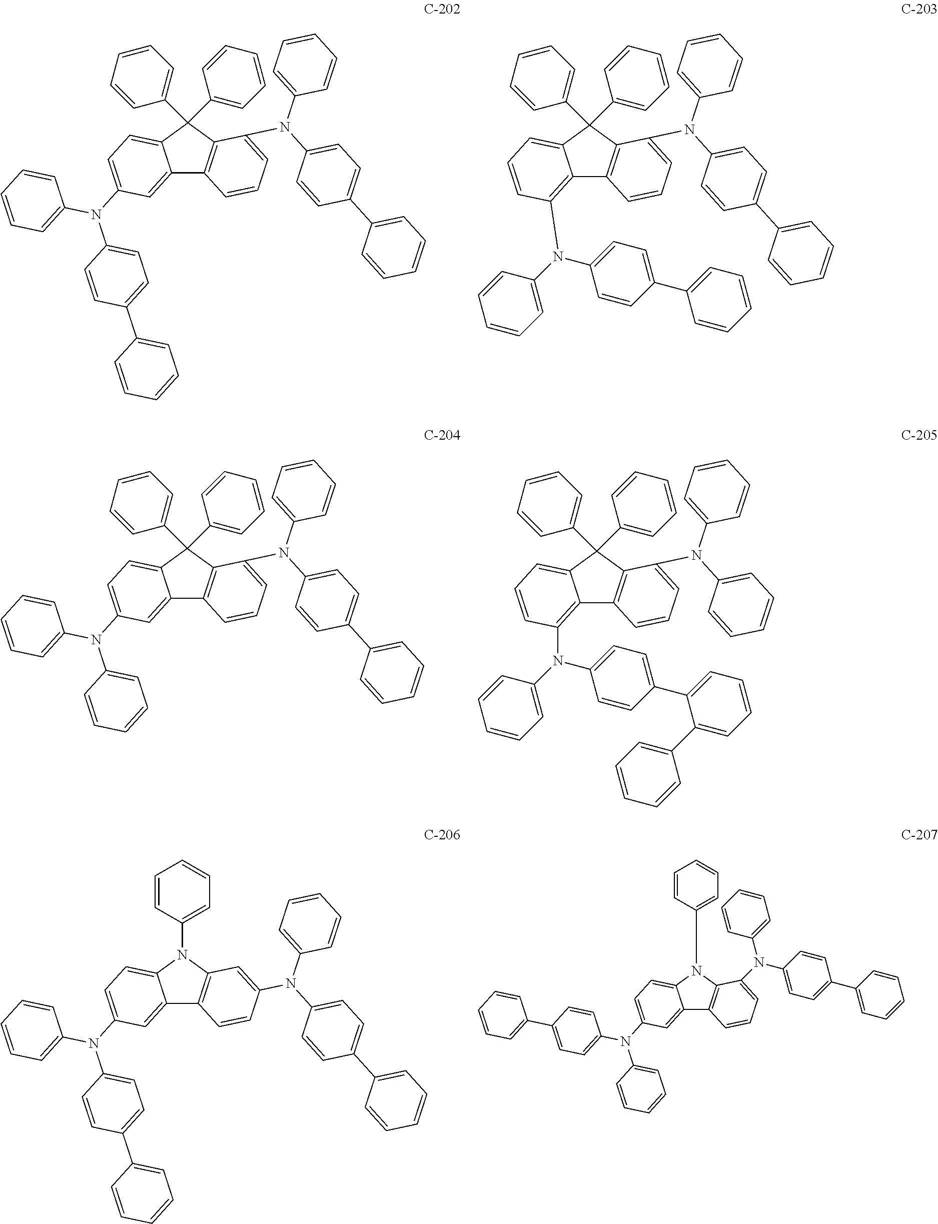 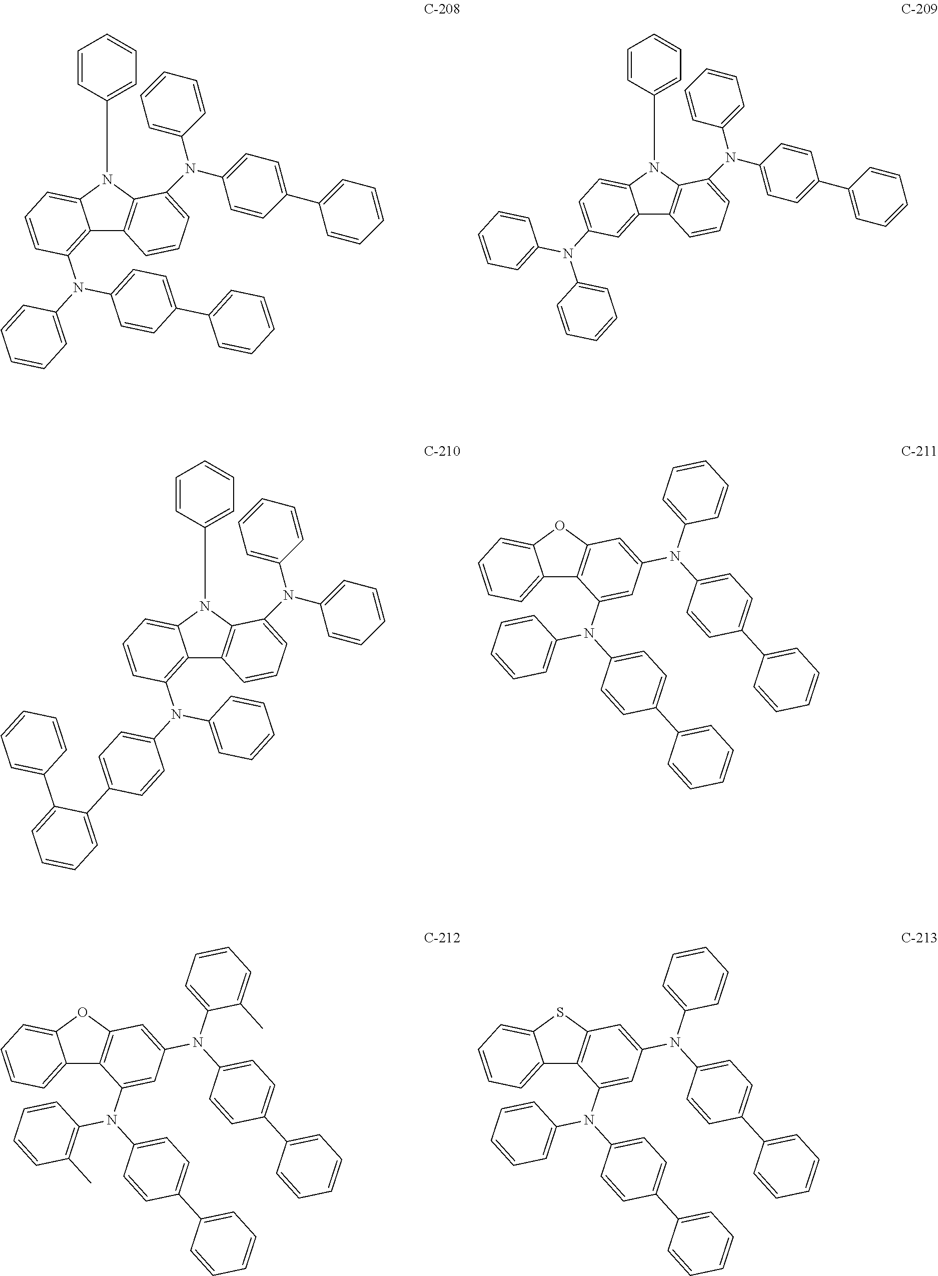 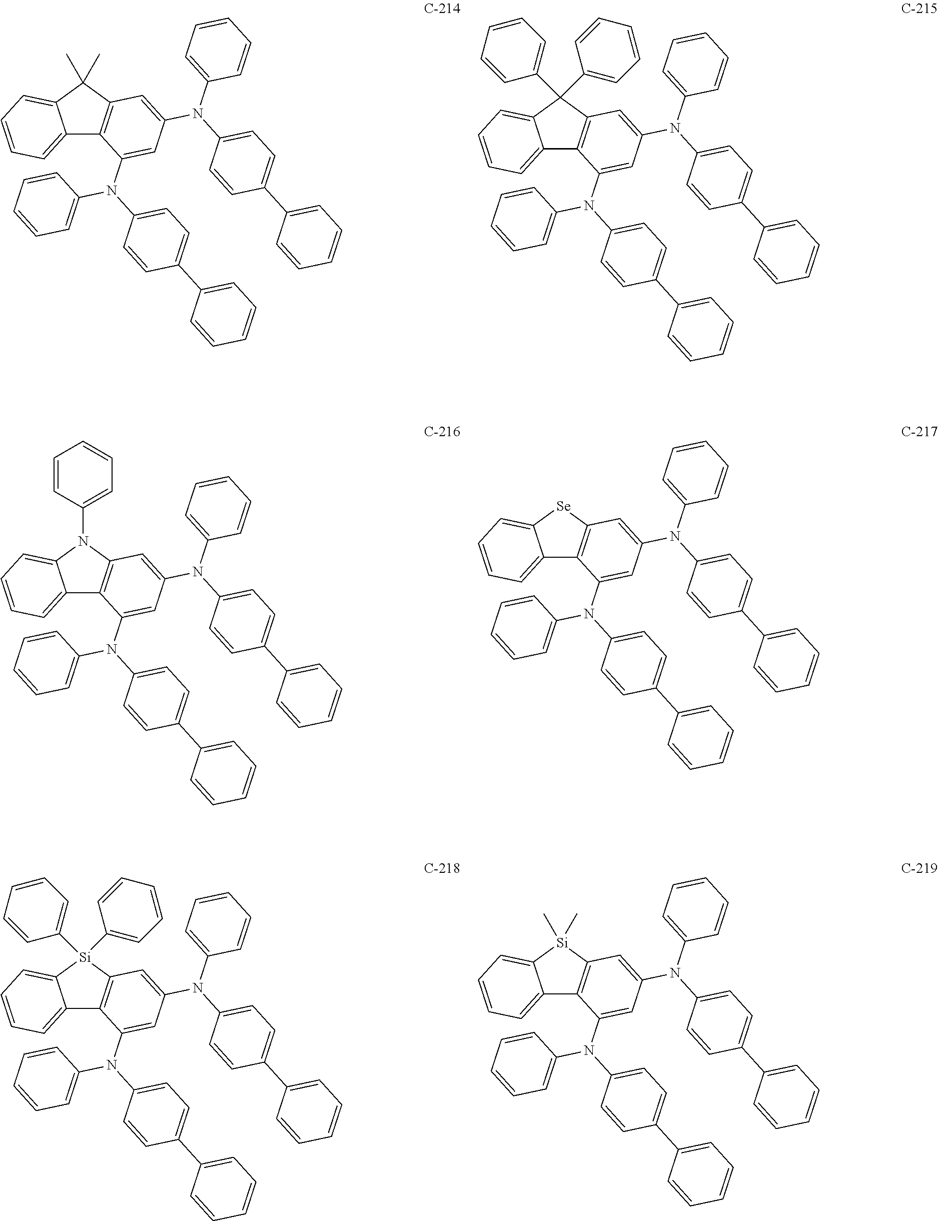 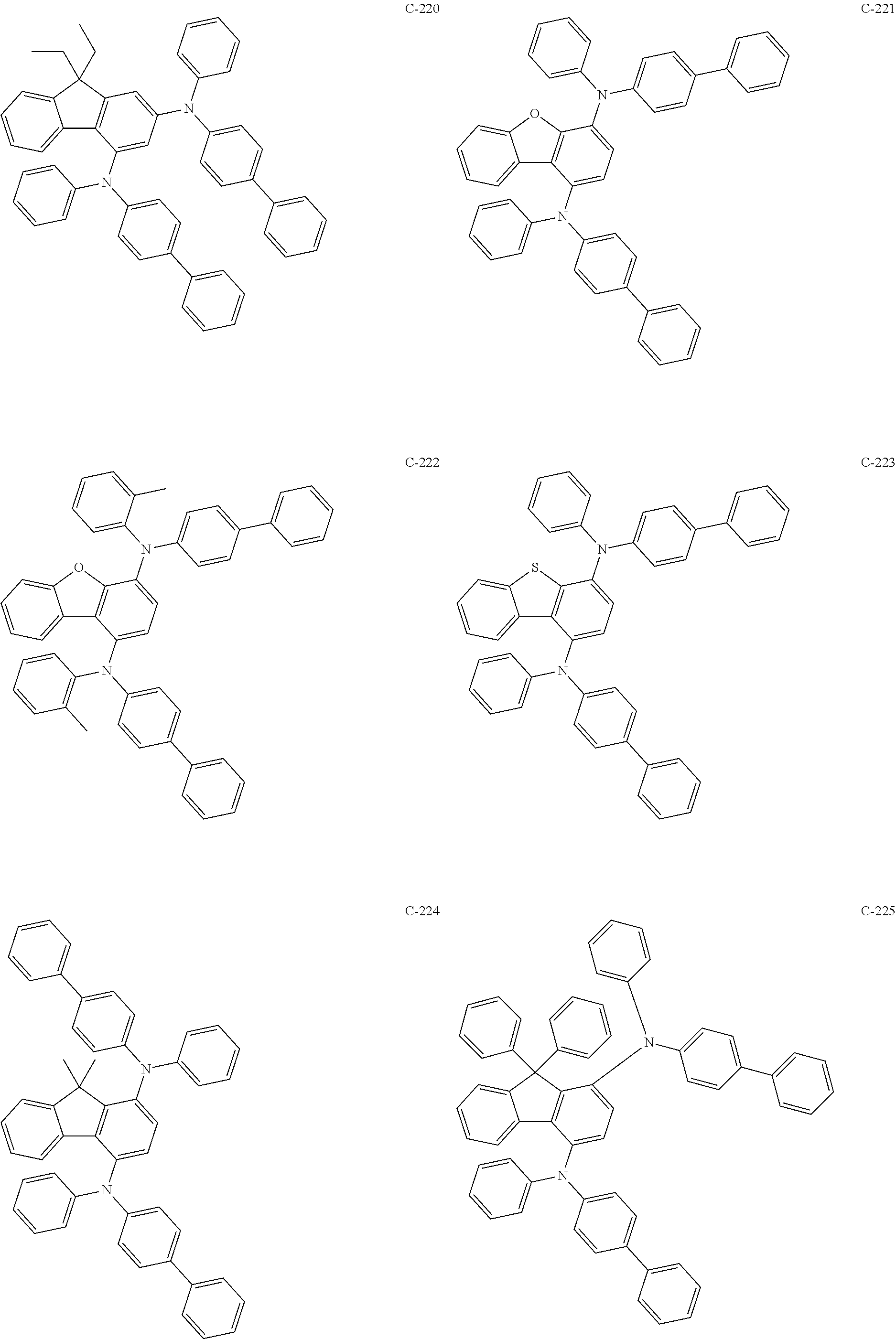 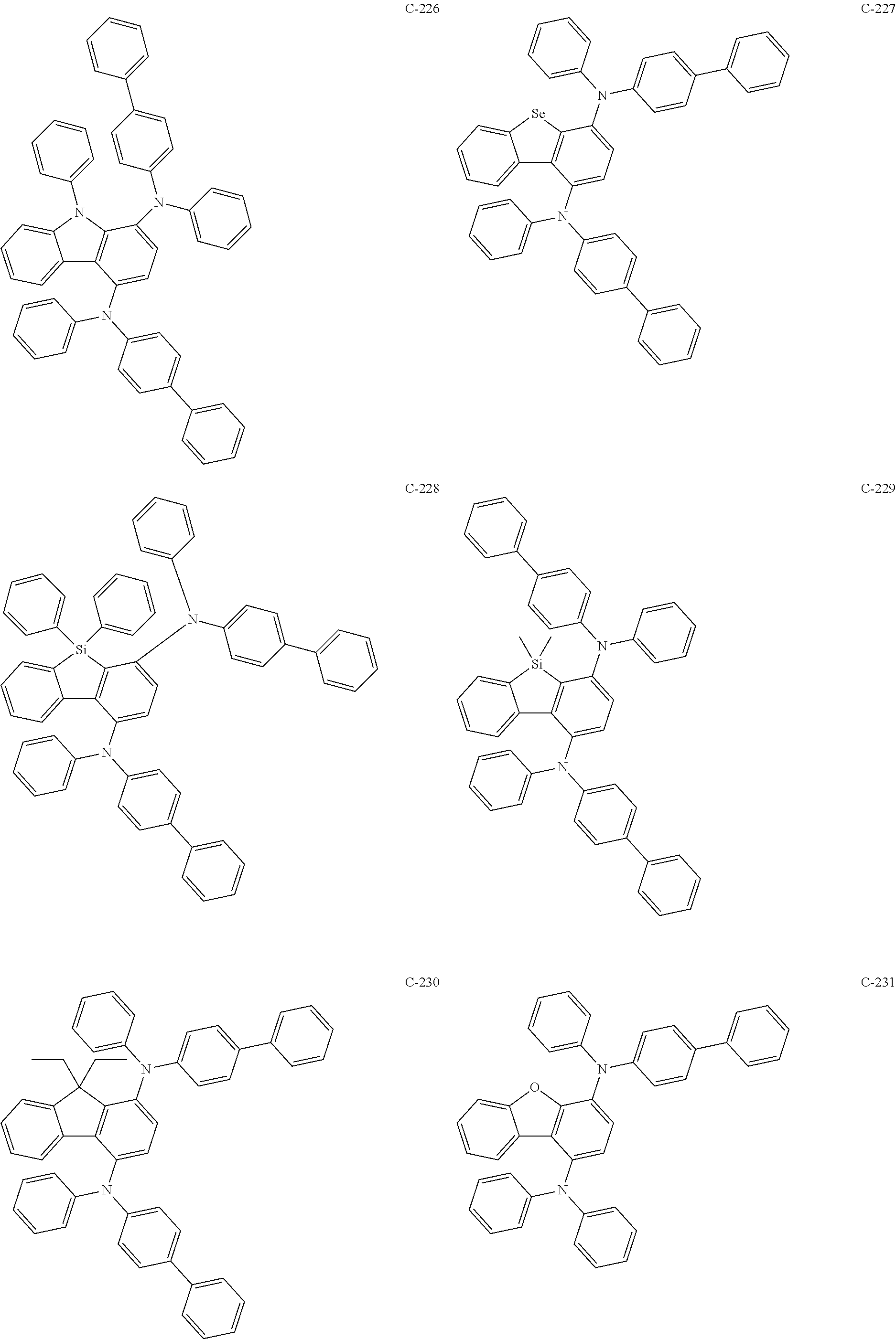 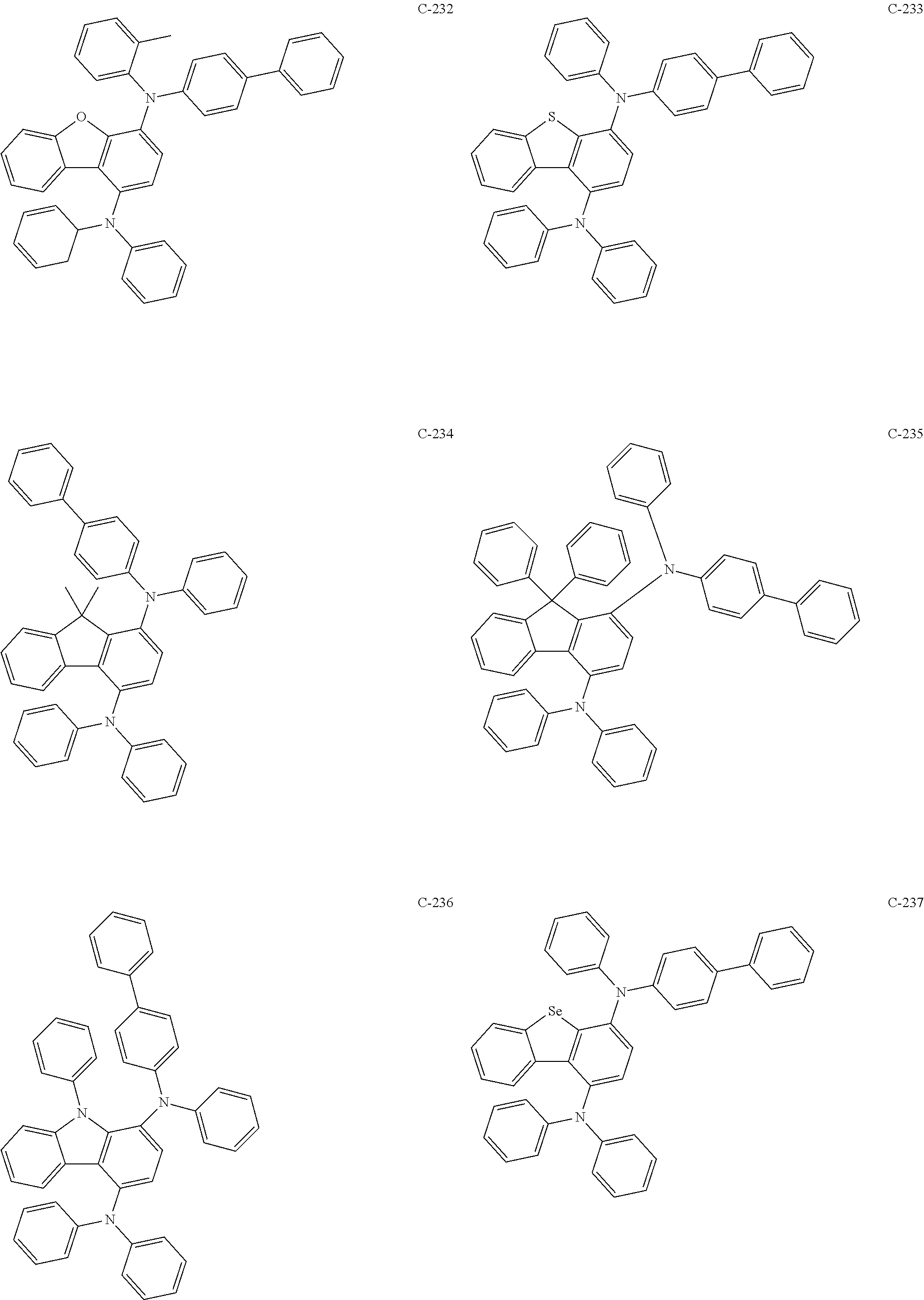 [SMILES: Cc1ccccc1N(c1ccc(-c2ccccc2)cc1)c1ccc(N(c2ccccc2)C2C=CC=CC2)c2c1oc1ccccc12 ; c1ccc(-c2ccc(N(c3ccccc3)c3ccc(N(c4ccccc4)c4ccccc4)c4c3sc3ccccc34)cc2)cc1 ; CC1(C)c2ccccc2-c2c(N(c3ccccc3)c3ccccc3)ccc(N(c3ccccc3)c3ccc(-c4ccccc4)cc3)c21 ; c1ccc(-c2ccc(N(c3ccccc3)c3ccc(N(c4ccccc4)c4ccccc4)c4c3C(c3ccccc3)(c3ccccc3)c3ccccc3-4)cc2)cc1 ; c1ccc(-c2ccc(N(c3ccccc3)c3ccc(N(c4ccccc4)c4ccccc4)c4c5ccccc5n(-c5ccccc5)c34)cc2)cc1 ; c1ccc(-c2ccc(N(c3ccccc3)c3ccc(N(c4ccccc4)c4ccccc4)c4c3[se]c3ccccc34)cc2)cc1] 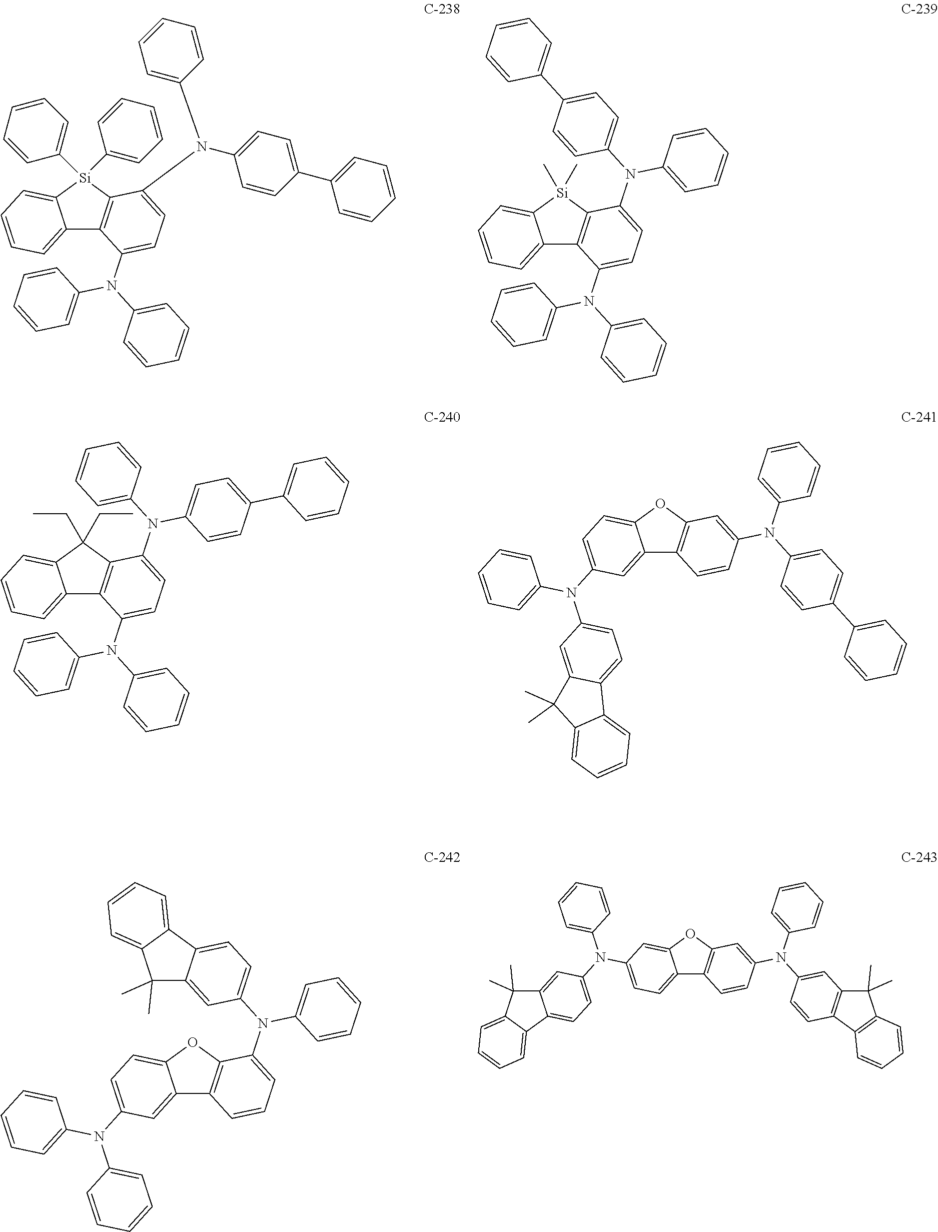 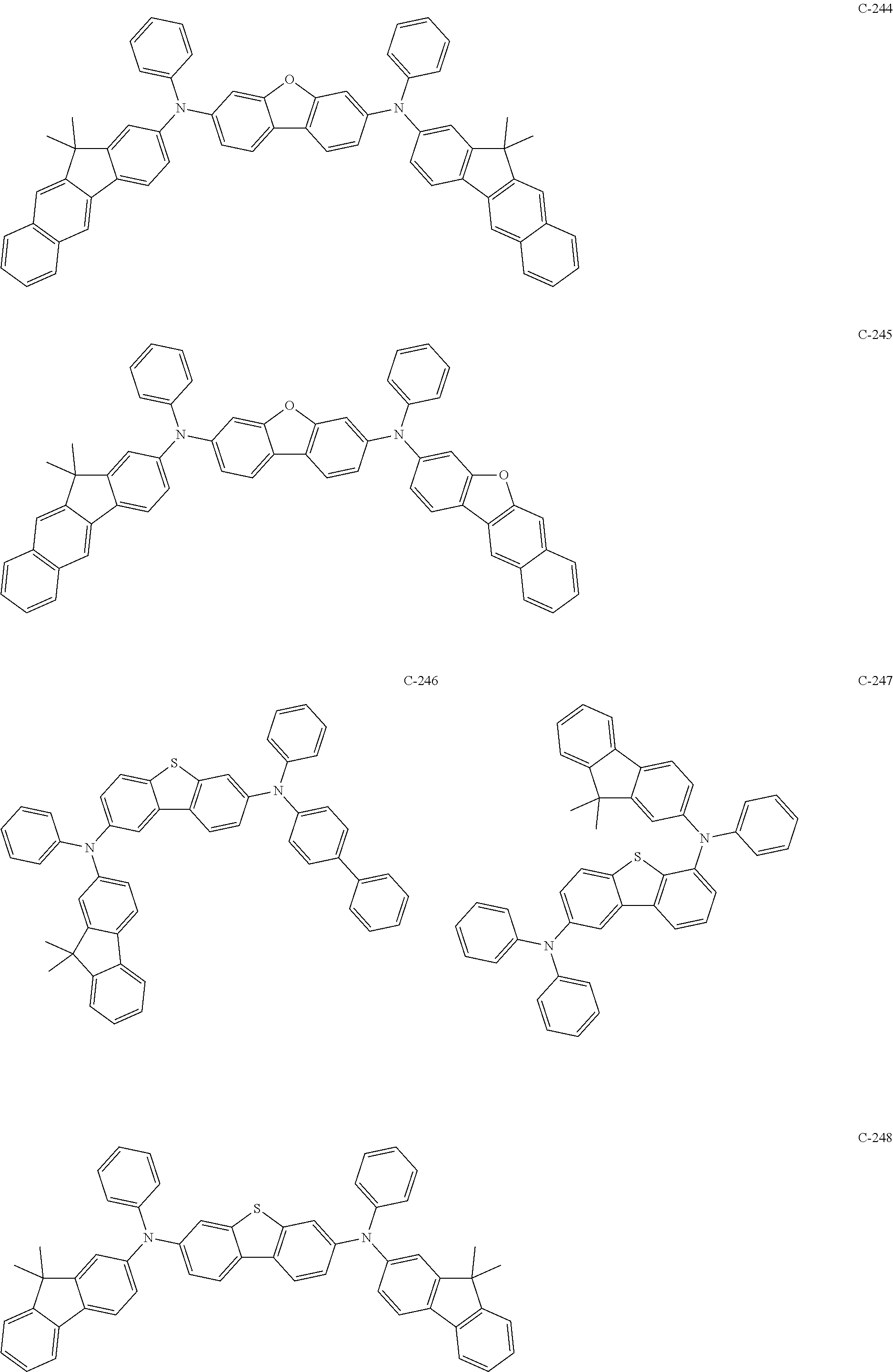 [SMILES: CC1(C)c2cc(N(c3ccccc3)c3ccc4c(c3)oc3cc(N(c5ccccc5)c5ccc6c(c5)C(C)(C)c5cc7ccccc7cc5-6)ccc34)ccc2-c2cc3ccccc3cc21 ; CC1(C)c2cc(N(c3ccccc3)c3ccc4c(c3)oc3cc(N(c5ccccc5)c5ccc6c(c5)oc5cc7ccccc7cc56)ccc34)ccc2-c2cc3ccccc3cc21 ; CC1(C)c2ccccc2-c2ccc(N(c3ccccc3)c3ccc4sc5cc(N(c6ccccc6)c6ccc(-c7ccccc7)cc6)ccc5c4c3)cc21 ; CC1(C)c2ccccc2-c2ccc(N(c3ccccc3)c3cccc4c3sc3ccc(N(c5ccccc5)c5ccccc5)cc34)cc21 ; CC1(C)c2ccccc2-c2ccc(N(c3ccccc3)c3ccc4c(c3)sc3cc(N(c5ccccc5)c5ccc6c(c5)C(C)(C)c5ccccc5-6)ccc34)cc21] 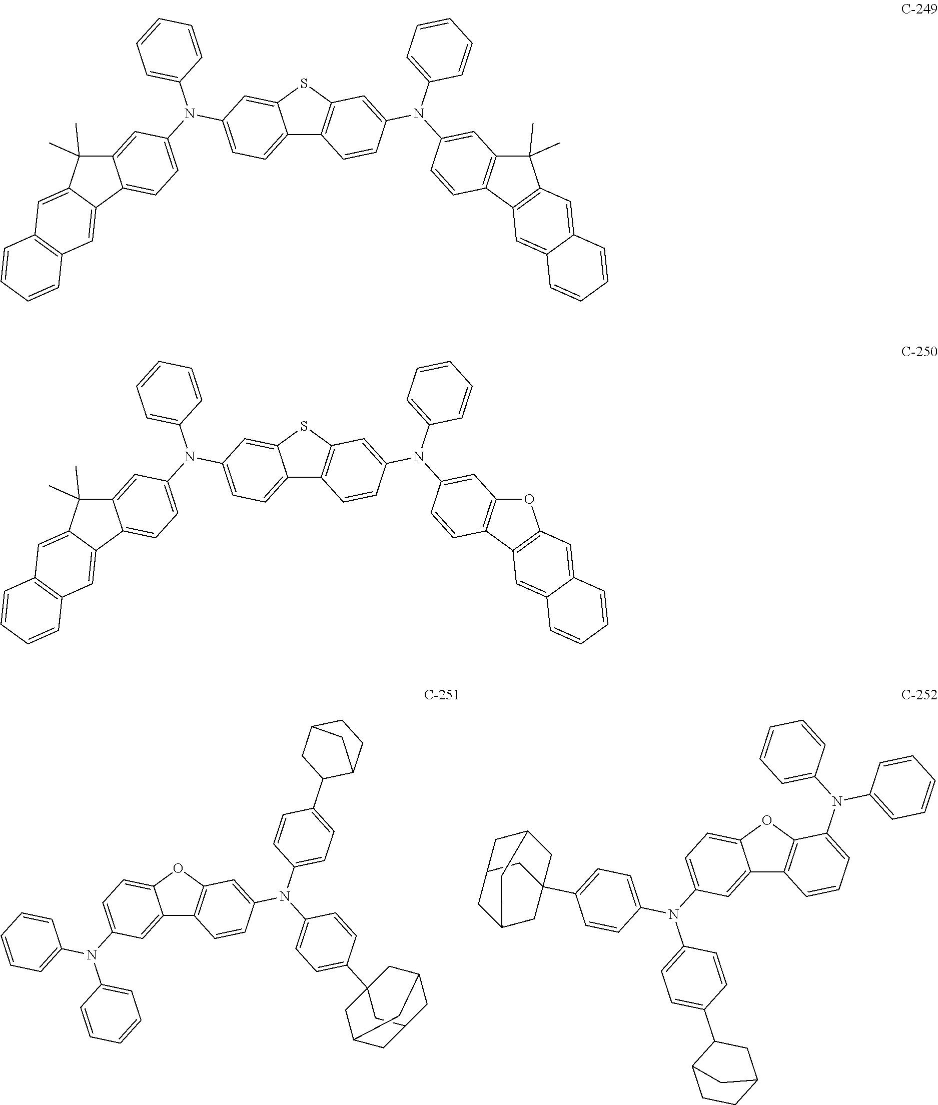 [SMILES: CC1(C)c2cc(N(c3ccccc3)c3ccc4c(c3)sc3cc(N(c5ccccc5)c5ccc6c(c5)C(C)(C)c5cc7ccccc7cc5-6)ccc34)ccc2-c2cc3ccccc3cc21 ; CC1(C)c2cc(N(c3ccccc3)c3ccc4c(c3)sc3cc(N(c5ccccc5)c5ccc6c(c5)oc5cc7ccccc7cc56)ccc34)ccc2-c2cc3ccccc3cc21 ; c1ccc(N(c2ccccc2)c2ccc3oc4cc(N(c5ccc(C6CC7CCC6C7)cc5)c5ccc(C67CC8CC(CC(C8)C6)C7)cc5)ccc4c3c2)cc1 ; c1ccc(N(c2ccccc2)c2cccc3c2oc2ccc(N(c4ccc(C5CC6CCC5C6)cc4)c4ccc(C56CC7CC(CC(C7)C5)C6)cc4)cc23)cc1] 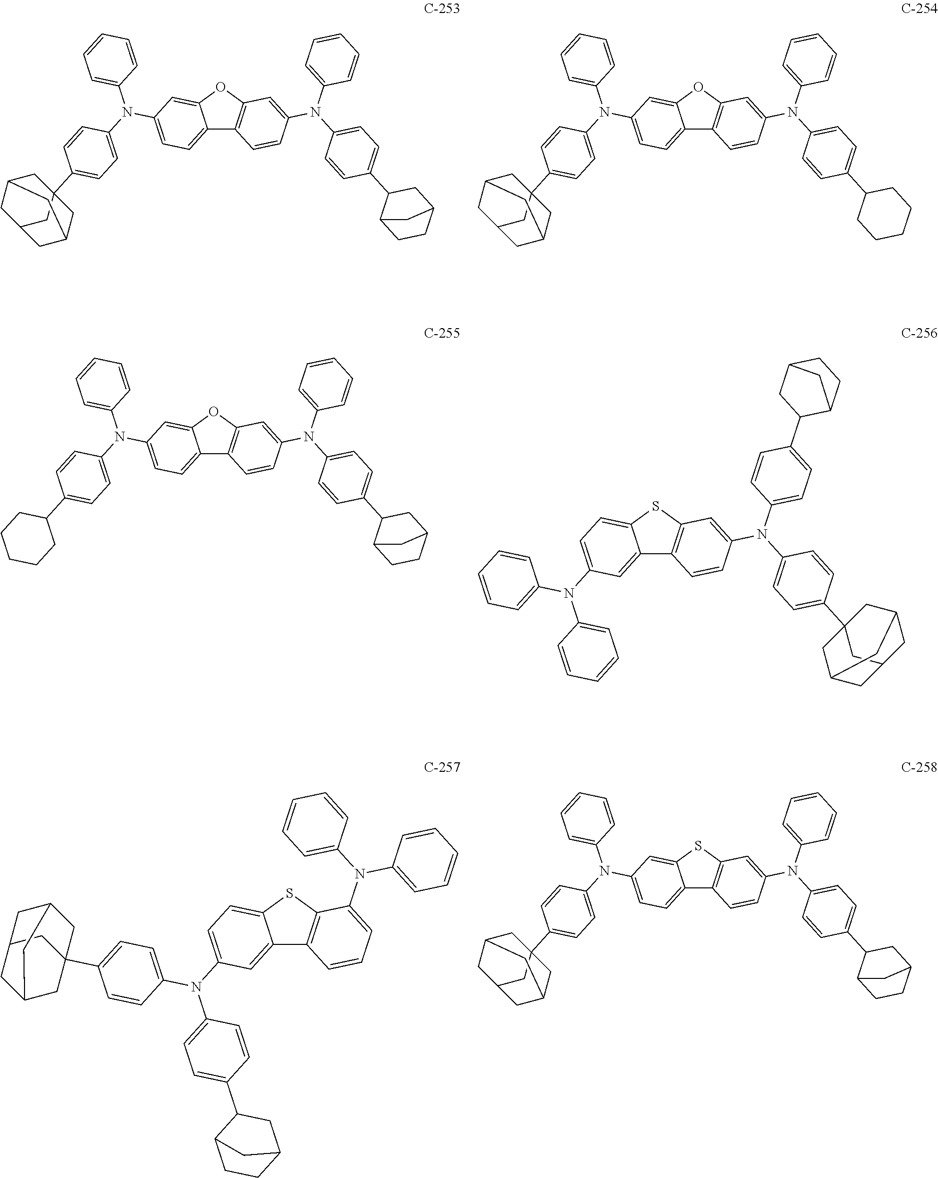 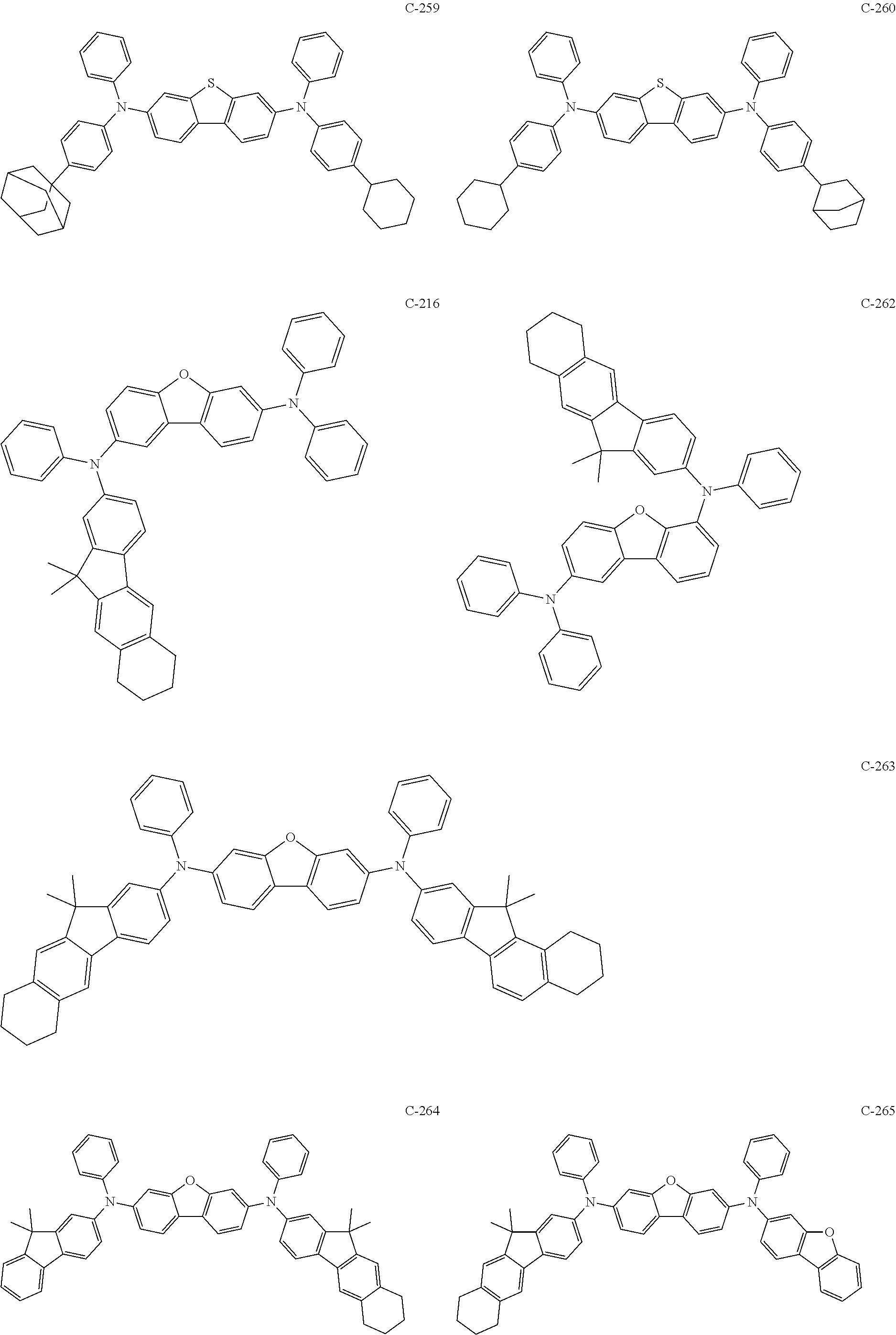 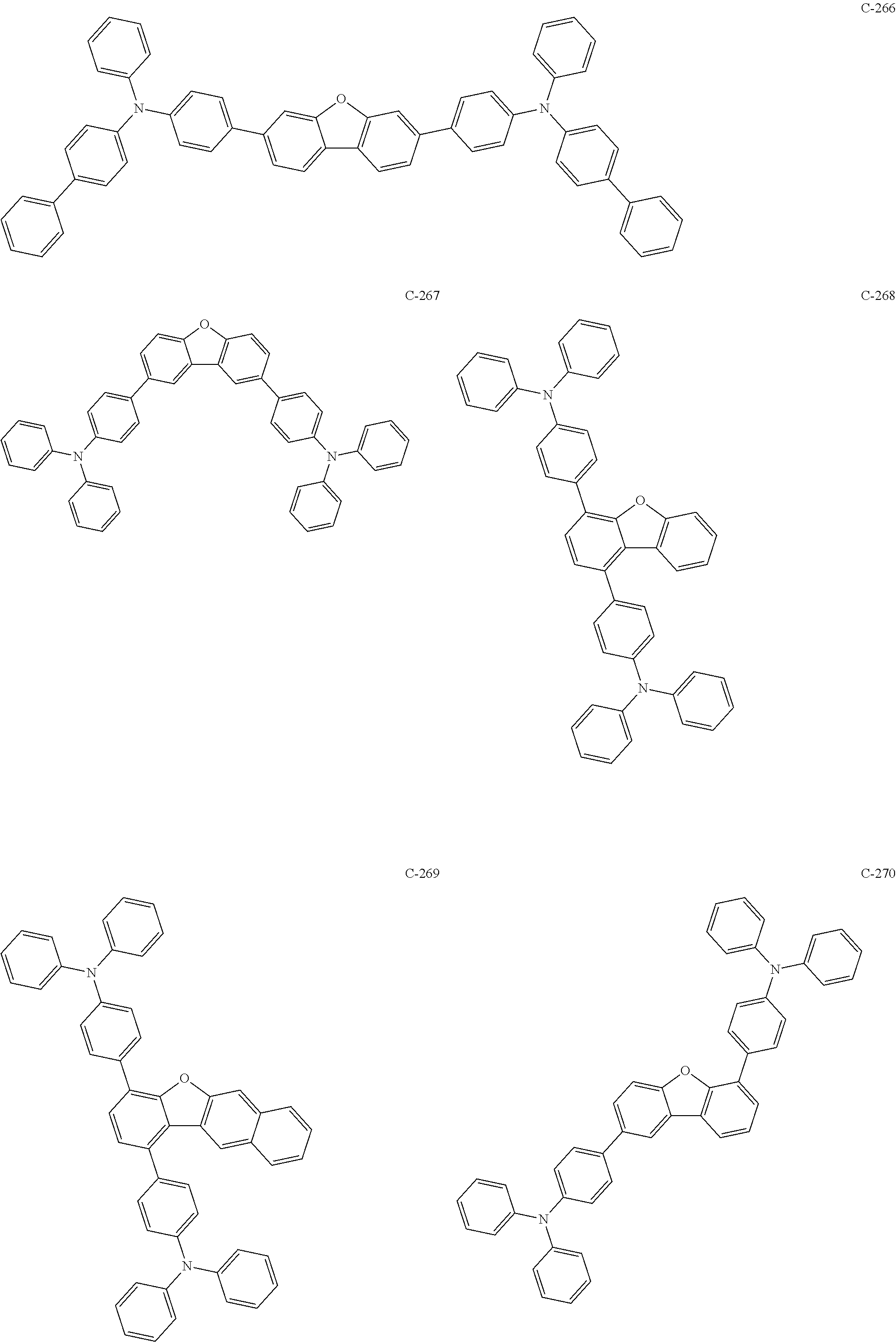 [SMILES: c1ccc(-c2ccc(N(c3ccccc3)c3ccc(-c4ccc5c(c4)oc4cc(-c6ccc(N(c7ccccc7)c7ccc(-c8ccccc8)cc7)cc6)ccc45)cc3)cc2)cc1 ; c1ccc(N(c2ccccc2)c2ccc(-c3ccc4oc5ccc(-c6ccc(N(c7ccccc7)c7ccccc7)cc6)cc5c4c3)cc2)cc1 ; c1ccc(N(c2ccccc2)c2ccc(-c3ccc(-c4ccc(N(c5ccccc5)c5ccccc5)cc4)c4c3oc3ccccc34)cc2)cc1 ; c1ccc(N(c2ccccc2)c2ccc(-c3ccc(-c4ccc(N(c5ccccc5)c5ccccc5)cc4)c4c3oc3cc5ccccc5cc34)cc2)cc1 ; c1ccc(N(c2ccccc2)c2ccc(-c3ccc4oc5c(-c6ccc(N(c7ccccc7)c7ccccc7)cc6)cccc5c4c3)cc2)cc1] 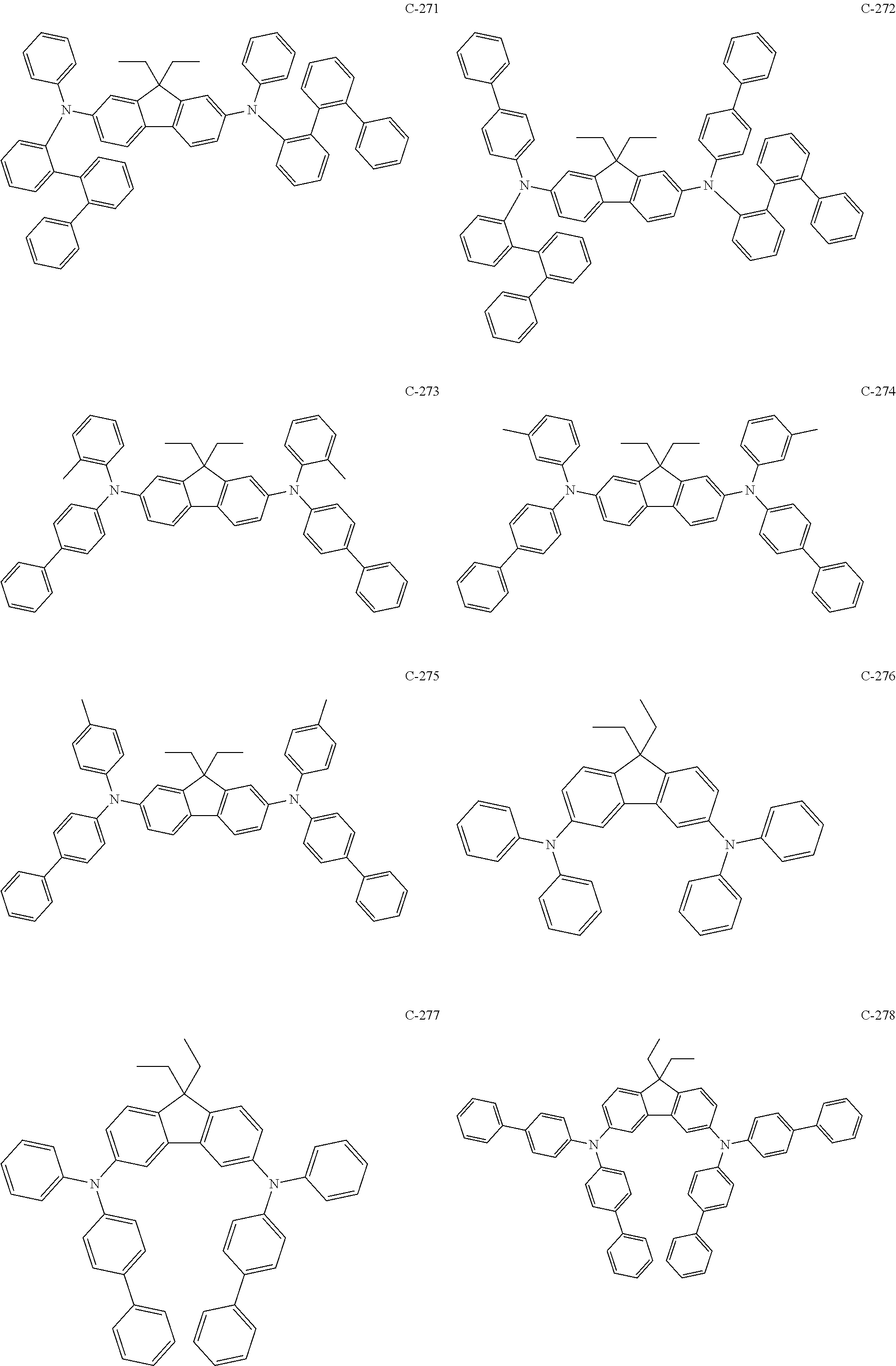 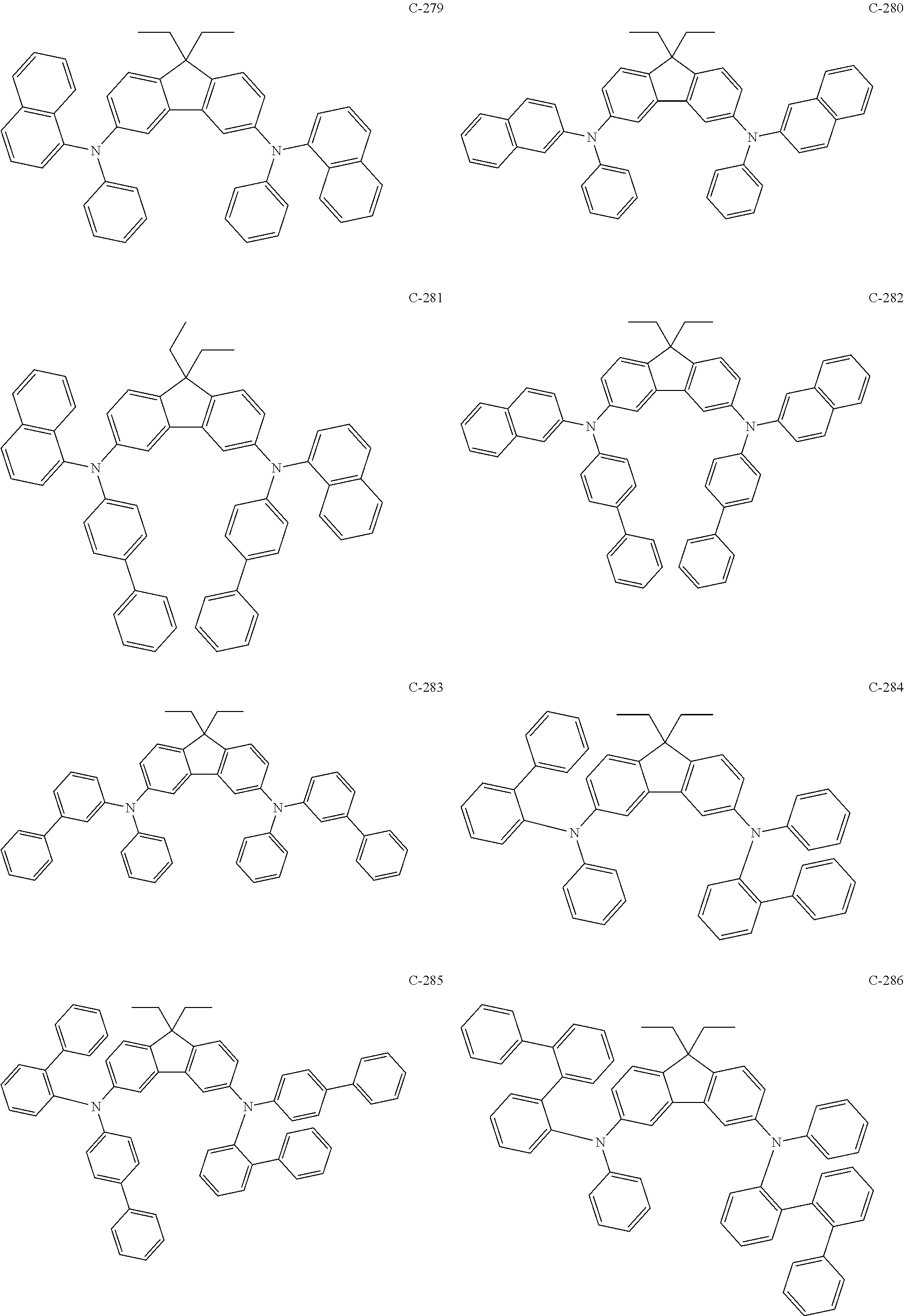 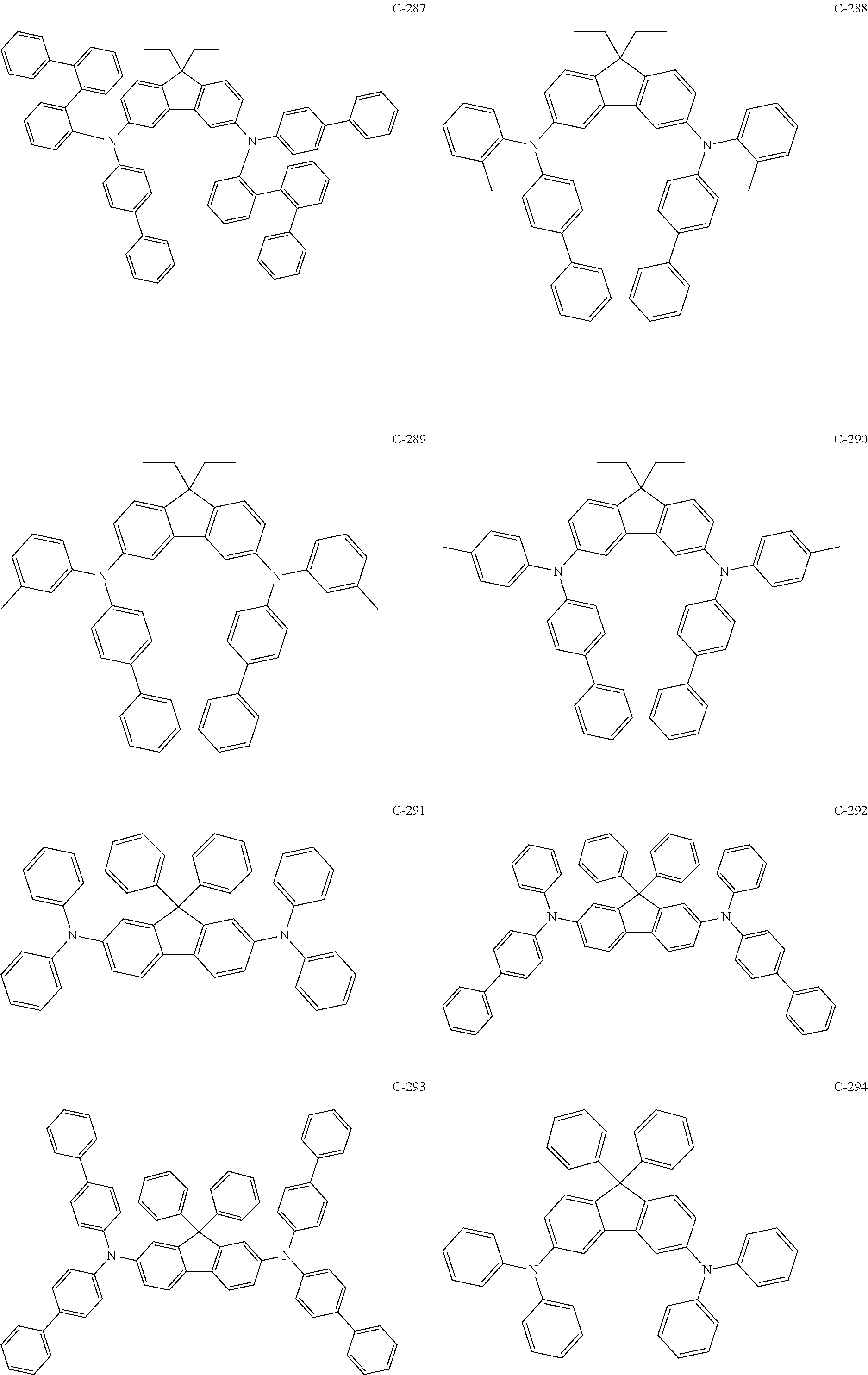 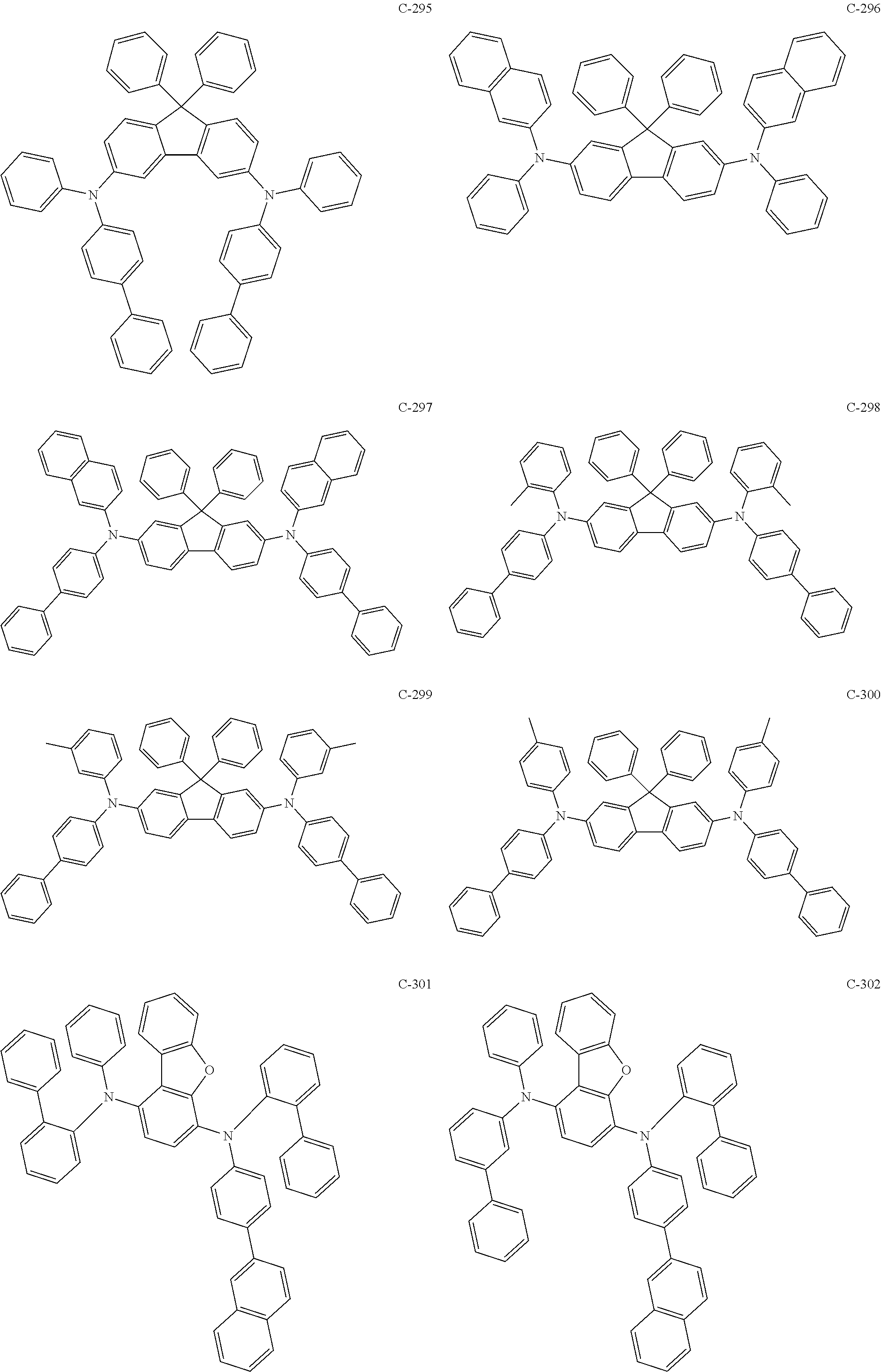 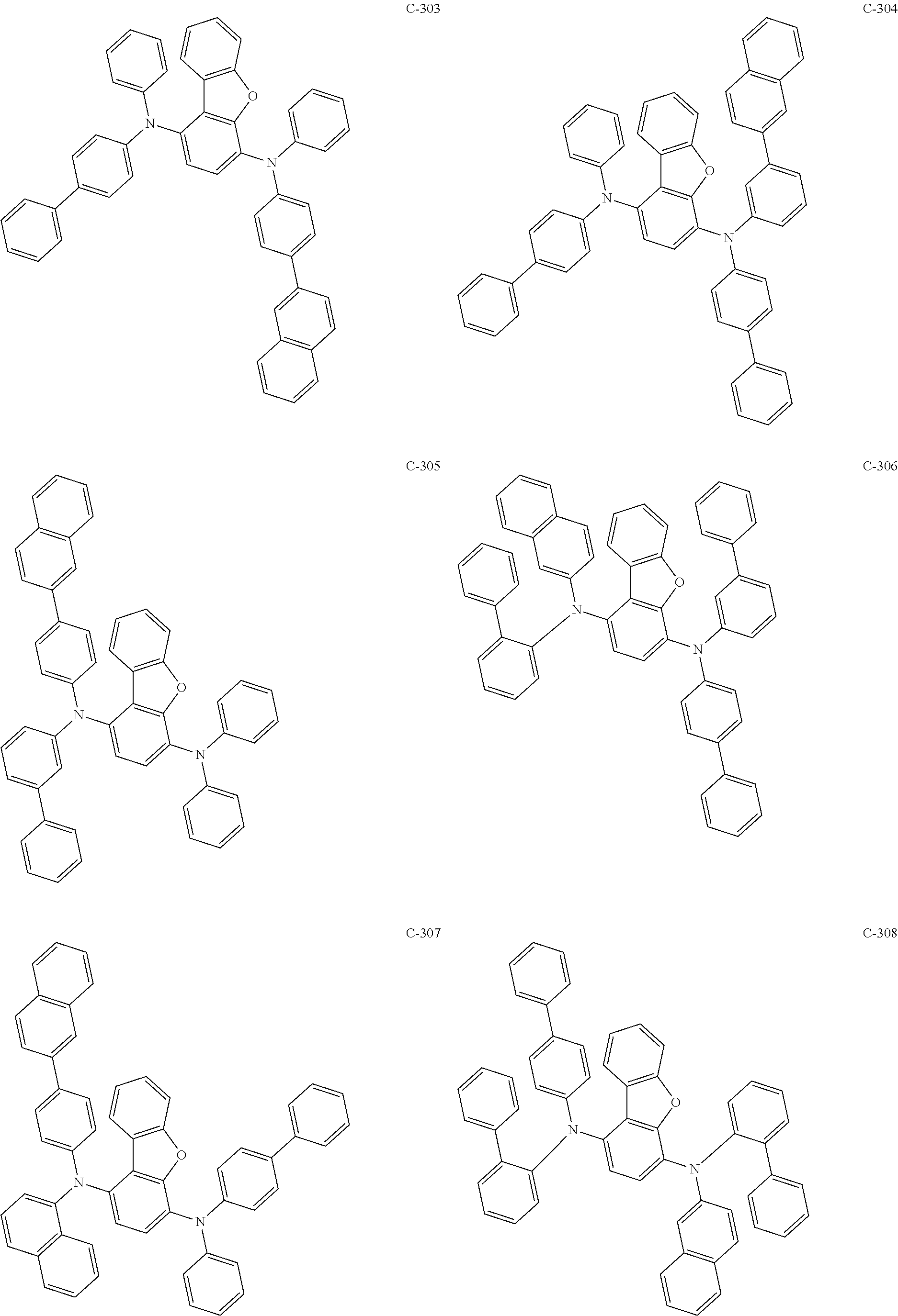 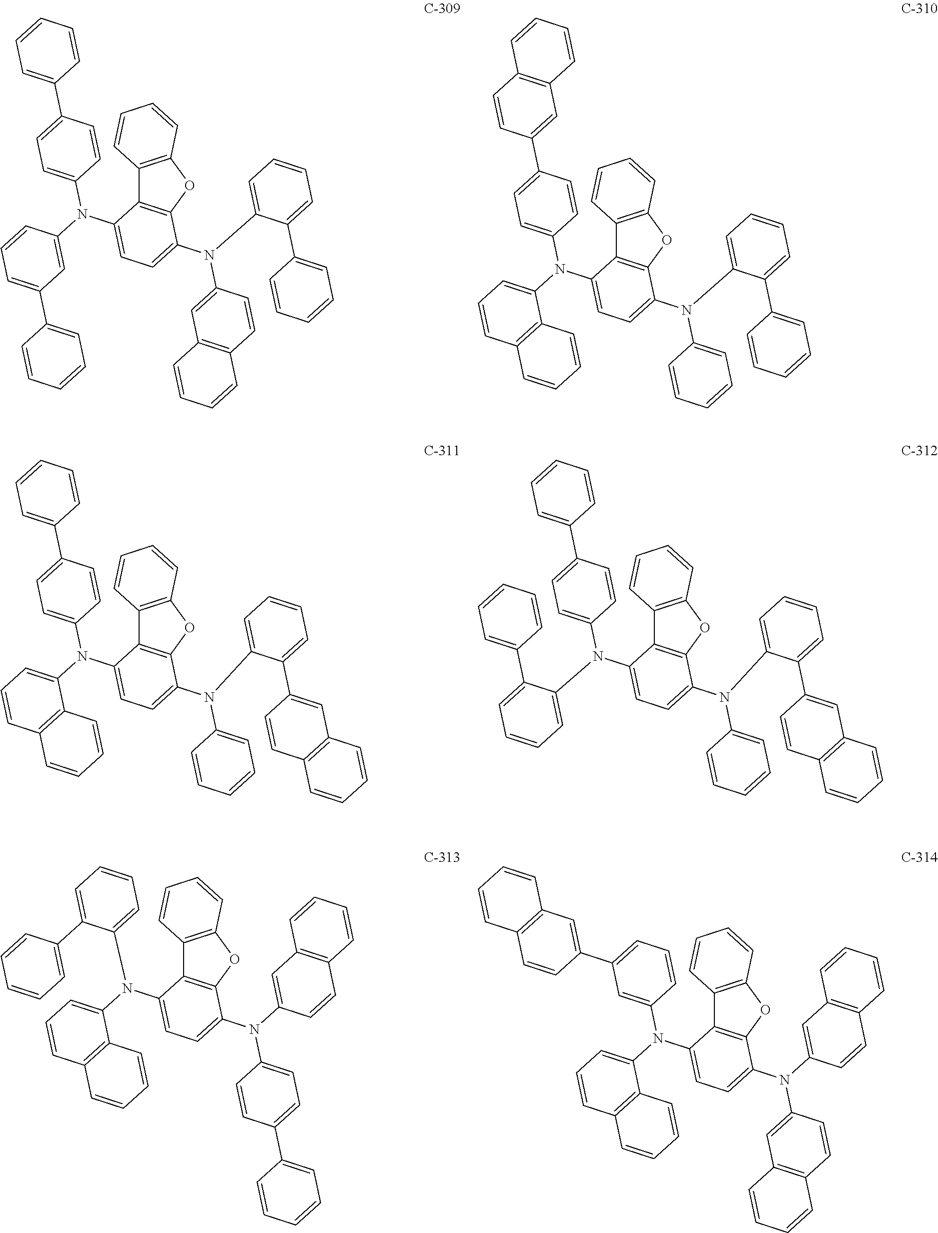 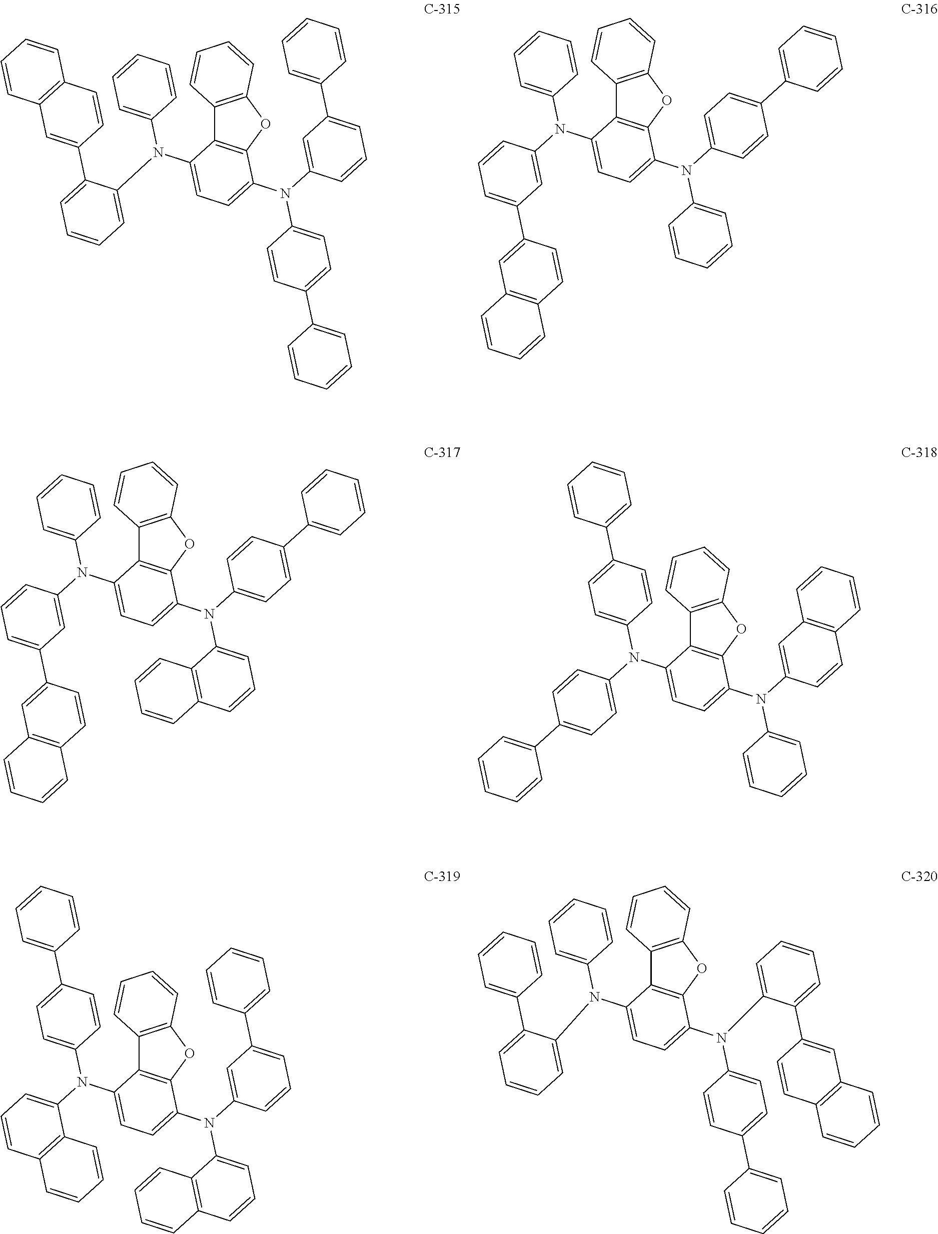 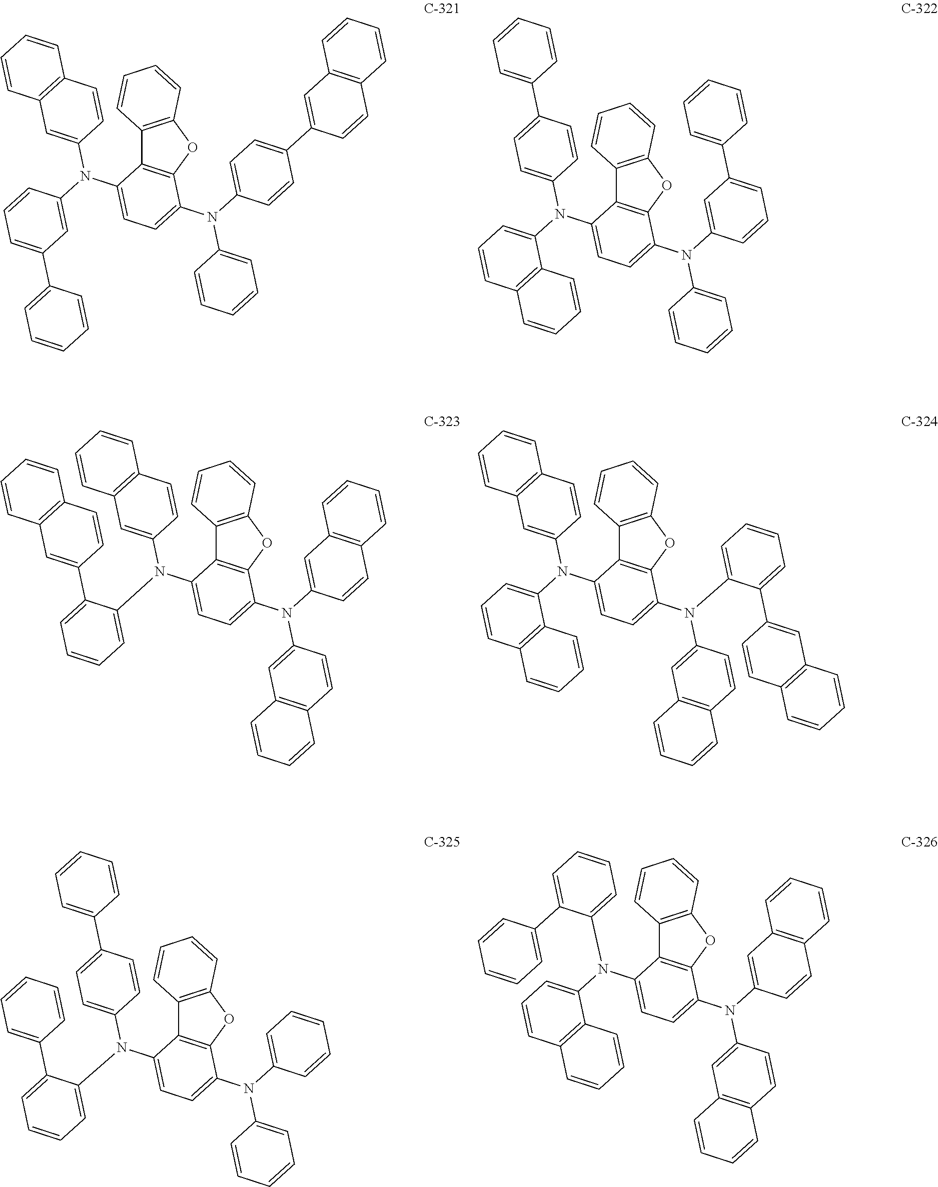 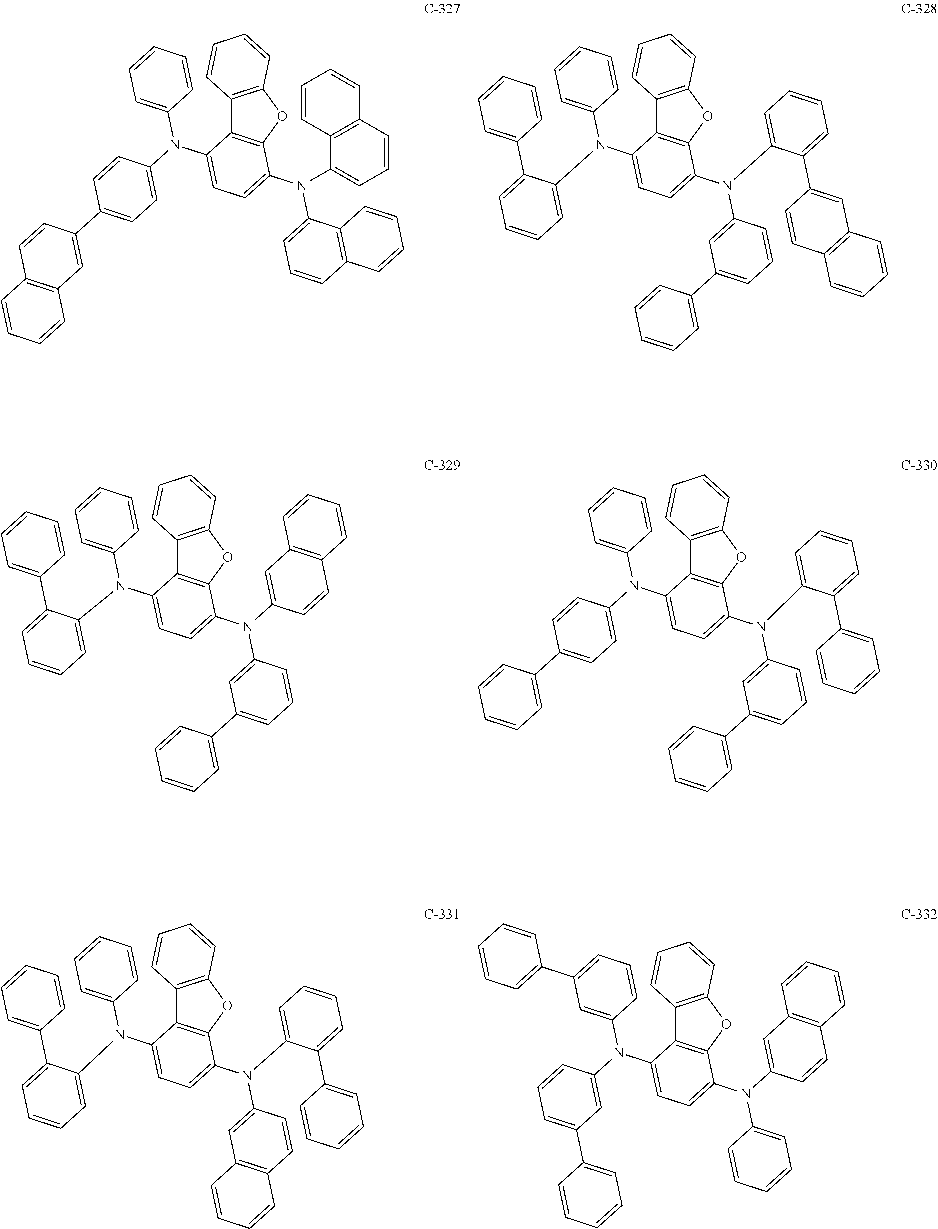 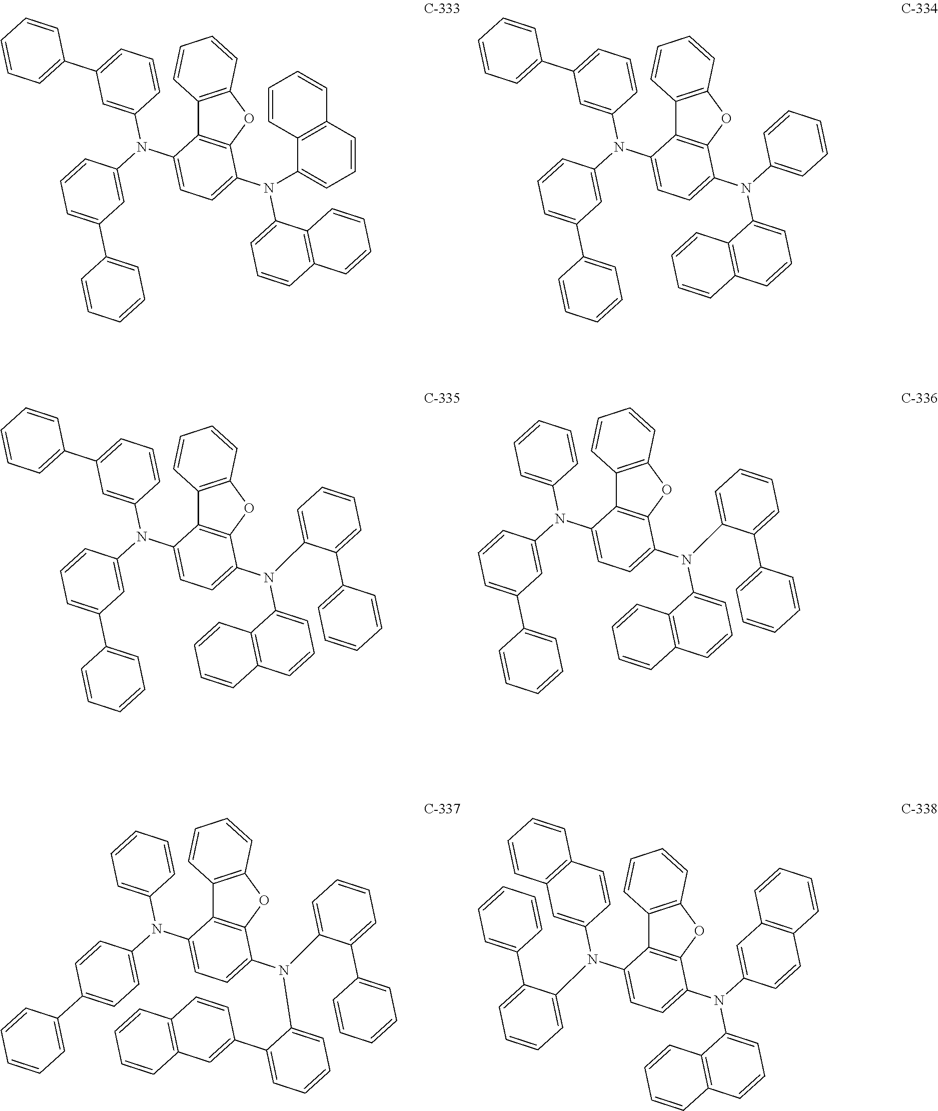 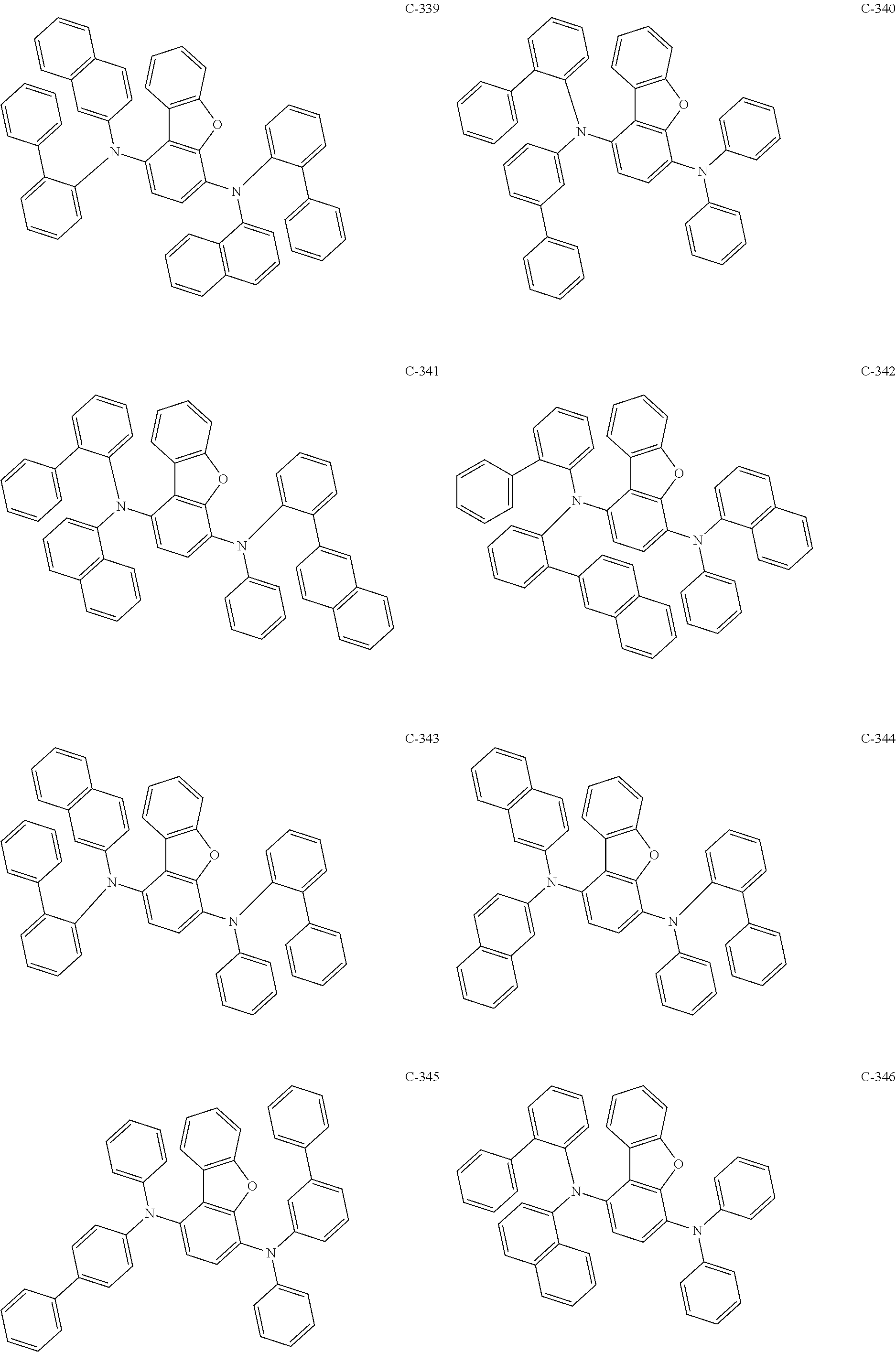 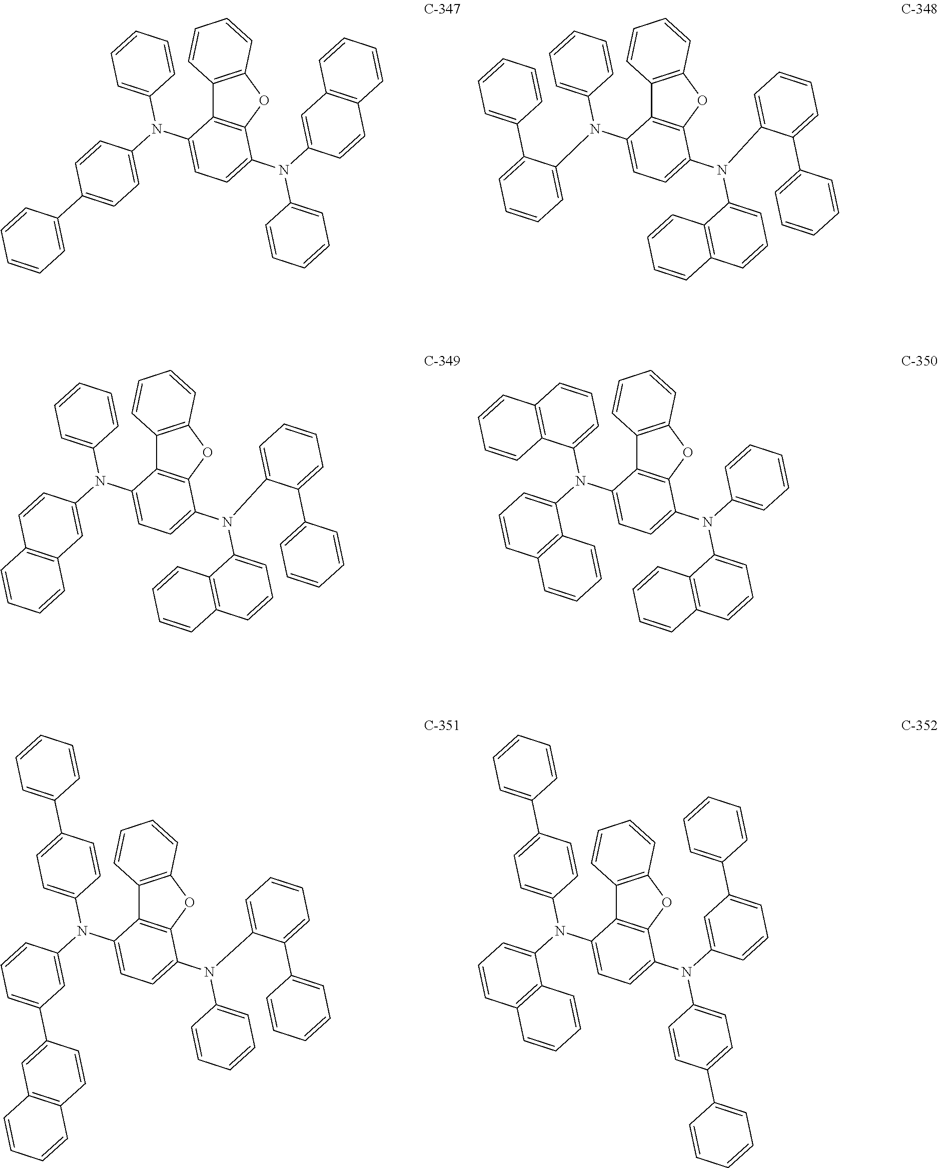 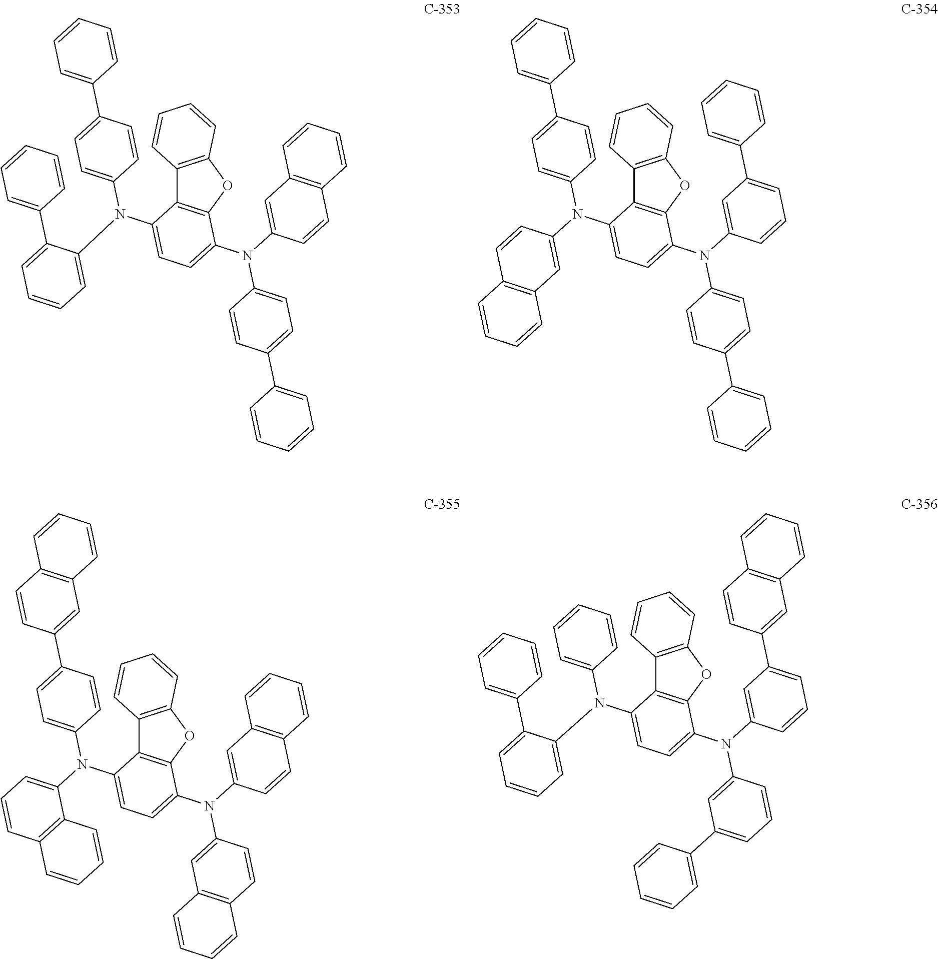 [SMILES: c1ccc(-c2ccc(N(c3ccc4ccccc4c3)c3ccc(N(c4ccc(-c5ccccc5)cc4)c4ccccc4-c4ccccc4)c4c3oc3ccccc34)cc2)cc1 ; c1ccc(-c2ccc(N(c3cccc(-c4ccccc4)c3)c3ccc(N(c4ccc(-c5ccccc5)cc4)c4ccc5ccccc5c4)c4c3oc3ccccc34)cc2)cc1 ; c1ccc2cc(-c3ccc(N(c4cccc5ccccc45)c4ccc(N(c5ccc6ccccc6c5)c5ccc6ccccc6c5)c5oc6ccccc6c45)cc3)ccc2c1 ; c1ccc(-c2cccc(N(c3cccc(-c4ccc5ccccc5c4)c3)c3ccc(N(c4ccccc4)c4ccccc4-c4ccccc4)c4c3oc3ccccc34)c2)cc1] 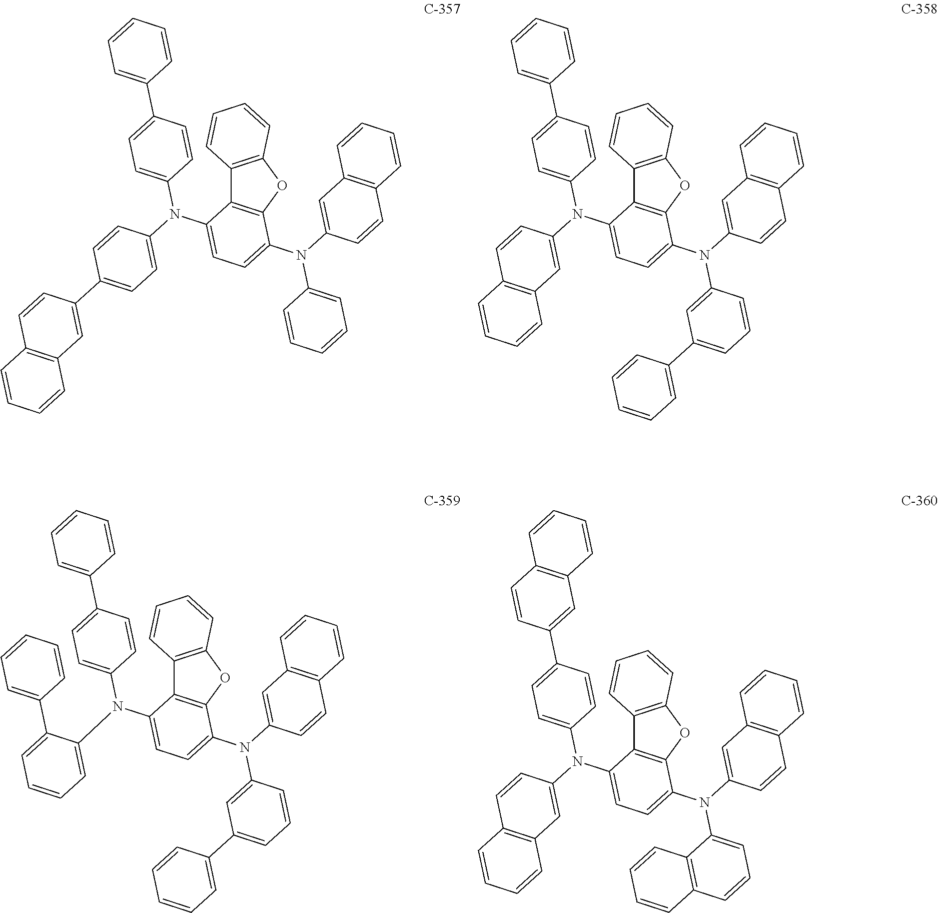 [SMILES: c1ccc(-c2ccc(N(c3ccc(-c4ccc5ccccc5c4)cc3)c3ccc(N(c4ccccc4)c4ccc5ccccc5c4)c4oc5ccccc5c34)cc2)cc1 ; c1ccc(-c2ccc(N(c3ccc4ccccc4c3)c3ccc(N(c4cccc(-c5ccccc5)c4)c4ccc5ccccc5c4)c4oc5ccccc5c34)cc2)cc1 ; c1ccc(-c2ccc(N(c3ccccc3-c3ccccc3)c3ccc(N(c4cccc(-c5ccccc5)c4)c4ccc5ccccc5c4)c4oc5ccccc5c34)cc2)cc1 ; c1ccc2cc(-c3ccc(N(c4ccc5ccccc5c4)c4ccc(N(c5ccc6ccccc6c5)c5cccc6ccccc56)c5oc6ccccc6c45)cc3)ccc2c1] 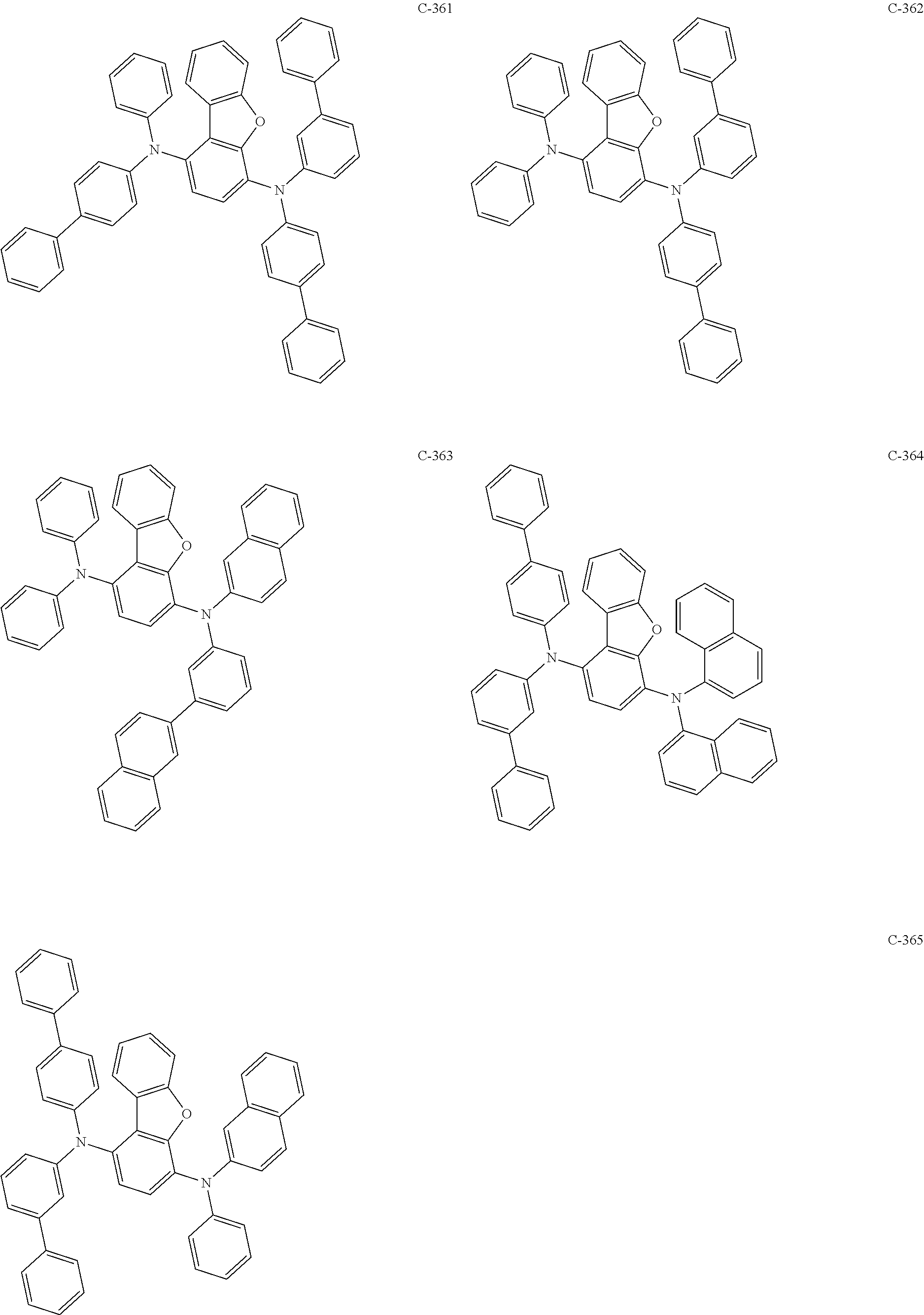 [SMILES: c1ccc(-c2ccc(N(c3cccc(-c4ccccc4)c3)c3ccc(N(c4ccccc4)c4ccc(-c5ccccc5)cc4)c4c3oc3ccccc34)cc2)cc1 ; c1ccc(-c2ccc(N(c3cccc(-c4ccccc4)c3)c3ccc(N(c4ccccc4)c4ccccc4)c4c3oc3ccccc34)cc2)cc1 ; c1ccc(N(c2ccccc2)c2ccc(N(c3cccc(-c4ccc5ccccc5c4)c3)c3ccc4ccccc4c3)c3oc4ccccc4c23)cc1 ; c1ccc(-c2ccc(N(c3cccc(-c4ccccc4)c3)c3ccc(N(c4cccc5ccccc45)c4cccc5ccccc45)c4oc5ccccc5c34)cc2)cc1 ; c1ccc(-c2ccc(N(c3cccc(-c4ccccc4)c3)c3ccc(N(c4ccccc4)c4ccc5ccccc5c4)c4oc5ccccc5c34)cc2)cc1] 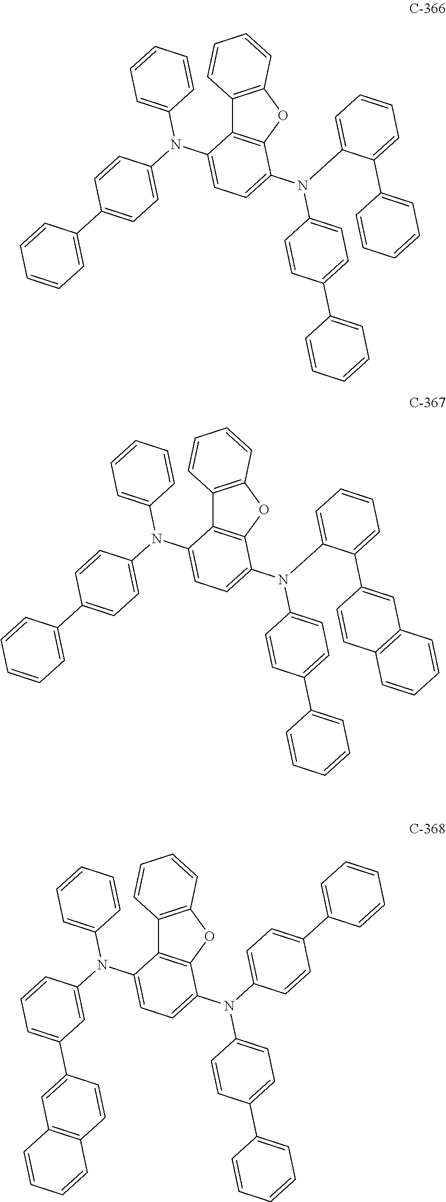 [SMILES: c1ccc(-c2ccc(N(c3ccccc3-c3ccccc3)c3ccc(N(c4ccccc4)c4ccc(-c5ccccc5)cc4)c4c3oc3ccccc34)cc2)cc1 ; c1ccc(-c2ccc(N(c3ccccc3-c3ccc4ccccc4c3)c3ccc(N(c4ccccc4)c4ccc(-c5ccccc5)cc4)c4c3oc3ccccc34)cc2)cc1 ; c1ccc(-c2ccc(N(c3ccc(-c4ccccc4)cc3)c3ccc(N(c4ccccc4)c4cccc(-c5ccc6ccccc6c5)c4)c4c3oc3ccccc34)cc2)cc1] 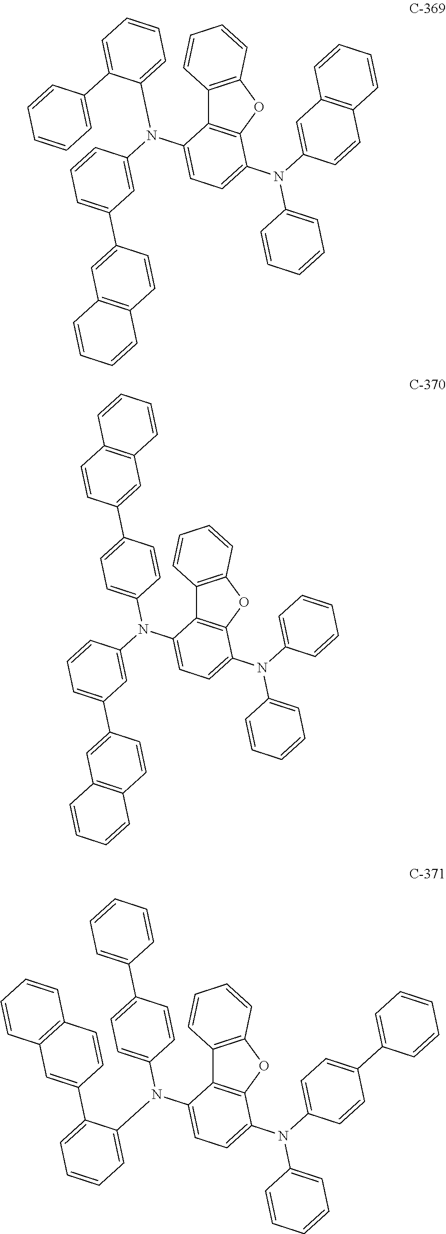 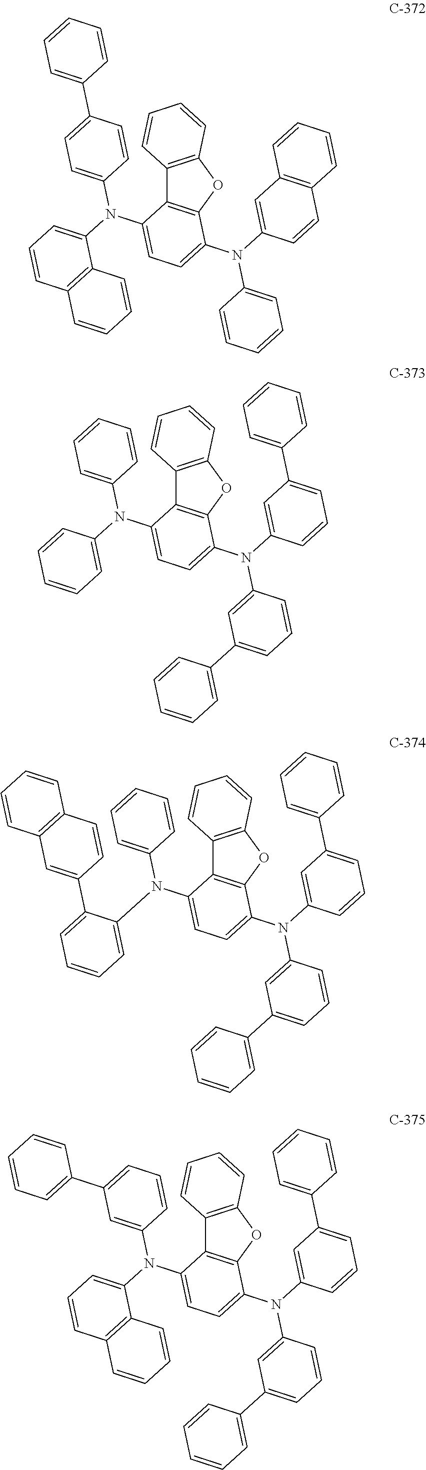 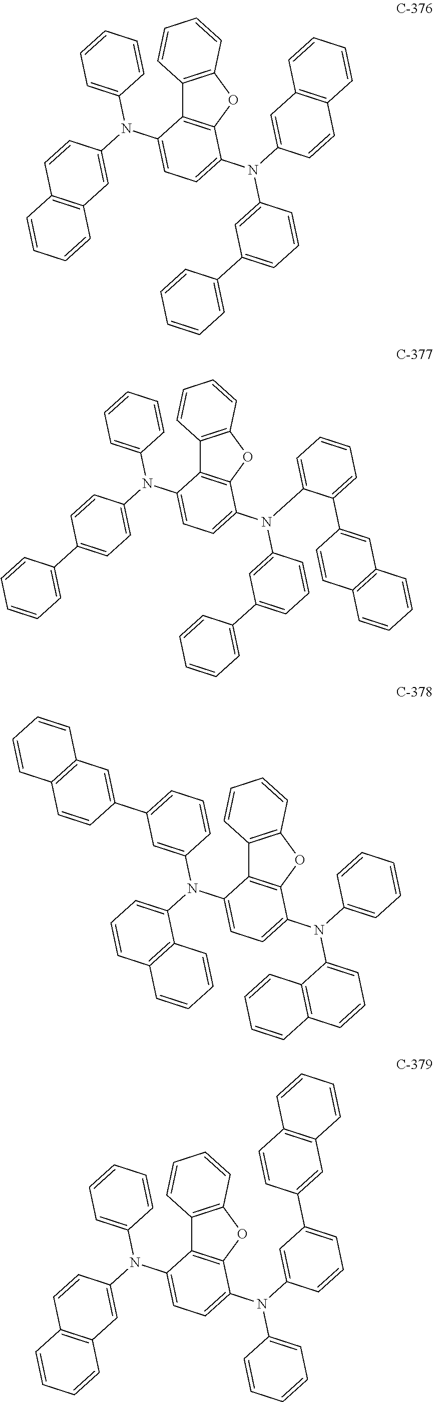 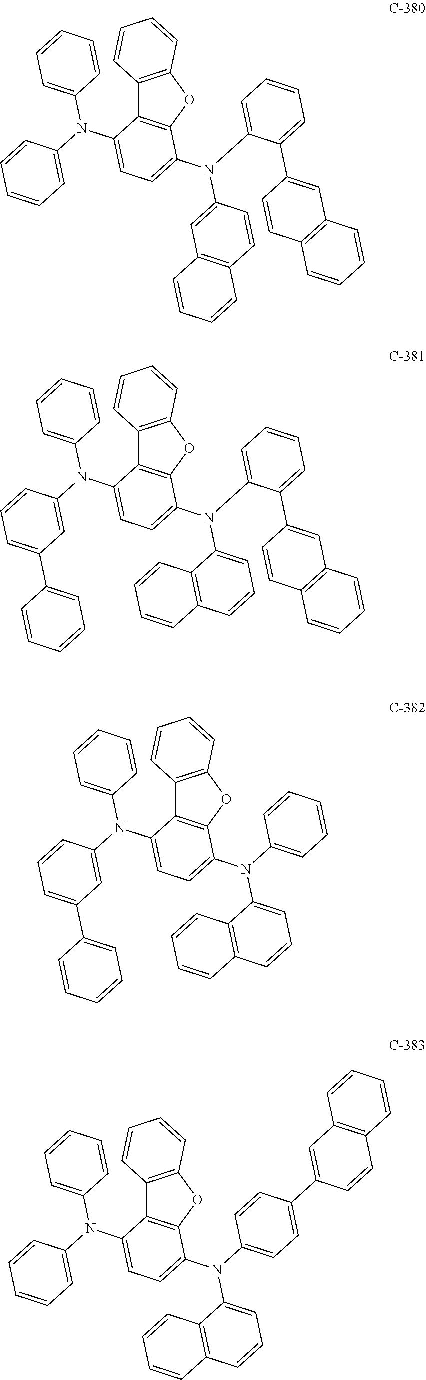 [SMILES: c1ccc(N(c2ccccc2)c2ccc(N(c3ccc4ccccc4c3)c3ccccc3-c3ccc4ccccc4c3)c3oc4ccccc4c23)cc1 ; c1ccc(-c2cccc(N(c3ccccc3)c3ccc(N(c4ccccc4-c4ccc5ccccc5c4)c4cccc5ccccc45)c4oc5ccccc5c34)c2)cc1 ; c1ccc(-c2cccc(N(c3ccccc3)c3ccc(N(c4ccccc4)c4cccc5ccccc45)c4oc5ccccc5c34)c2)cc1 ; c1ccc(N(c2ccccc2)c2ccc(N(c3ccc(-c4ccc5ccccc5c4)cc3)c3cccc4ccccc34)c3oc4ccccc4c23)cc1] 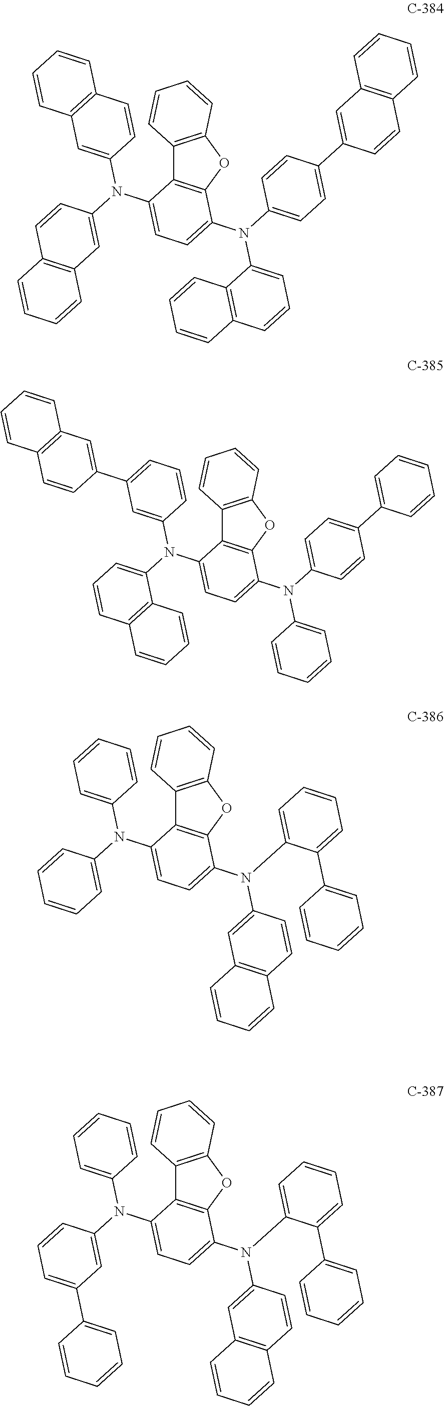 [SMILES: c1ccc2cc(-c3ccc(N(c4cccc5ccccc45)c4ccc(N(c5ccc6ccccc6c5)c5ccc6ccccc6c5)c5c4oc4ccccc45)cc3)ccc2c1 ; c1ccc(-c2ccc(N(c3ccccc3)c3ccc(N(c4cccc(-c5ccc6ccccc6c5)c4)c4cccc5ccccc45)c4c3oc3ccccc34)cc2)cc1 ; c1ccc(-c2ccccc2N(c2ccc3ccccc3c2)c2ccc(N(c3ccccc3)c3ccccc3)c3c2oc2ccccc23)cc1 ; c1ccc(-c2cccc(N(c3ccccc3)c3ccc(N(c4ccc5ccccc5c4)c4ccccc4-c4ccccc4)c4oc5ccccc5c34)c2)cc1] 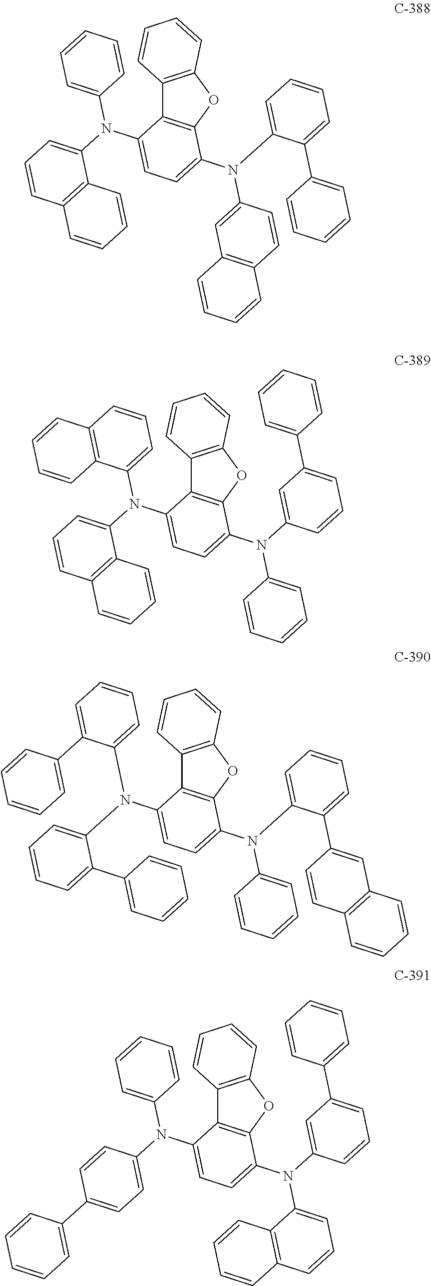 [SMILES: c1ccc(-c2ccccc2N(c2ccc3ccccc3c2)c2ccc(N(c3ccccc3)c3cccc4ccccc34)c3c2oc2ccccc23)cc1 ; c1ccc(-c2cccc(N(c3ccccc3)c3ccc(N(c4cccc5ccccc45)c4cccc5ccccc45)c4c3oc3ccccc34)c2)cc1 ; c1ccc(-c2ccccc2N(c2ccccc2-c2ccccc2)c2ccc(N(c3ccccc3)c3ccccc3-c3ccc4ccccc4c3)c3oc4ccccc4c23)cc1 ; c1ccc(-c2ccc(N(c3ccccc3)c3ccc(N(c4cccc(-c5ccccc5)c4)c4cccc5ccccc45)c4oc5ccccc5c34)cc2)cc1] 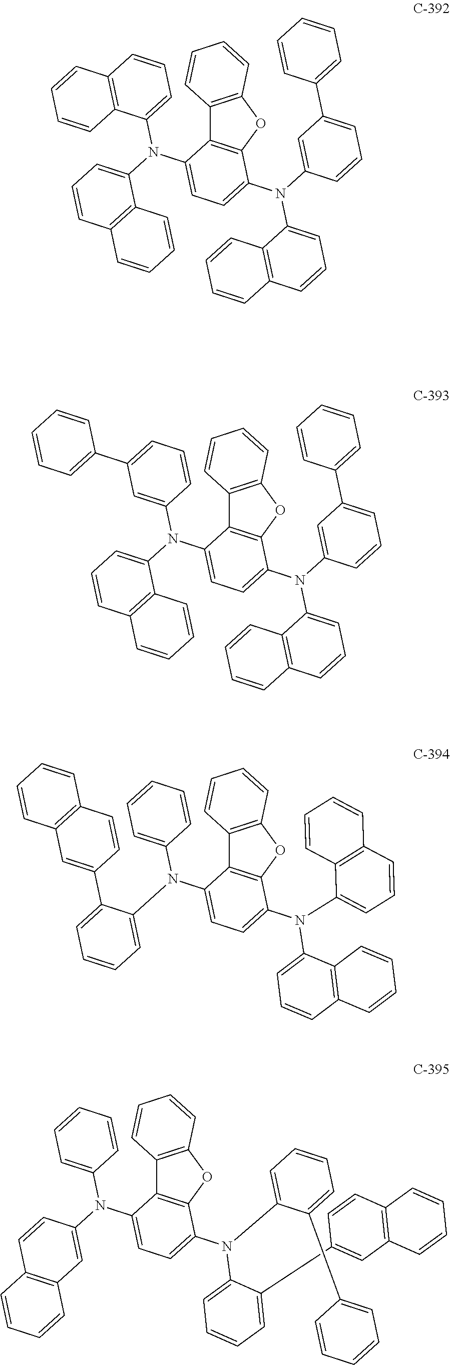 [SMILES: c1ccc(-c2cccc(N(c3cccc4ccccc34)c3ccc(N(c4cccc5ccccc45)c4cccc5ccccc45)c4c3oc3ccccc34)c2)cc1 ; c1ccc(-c2cccc(N(c3cccc4ccccc34)c3ccc(N(c4cccc(-c5ccccc5)c4)c4cccc5ccccc45)c4c3oc3ccccc34)c2)cc1 ; c1ccc(N(c2ccccc2-c2ccc3ccccc3c2)c2ccc(N(c3cccc4ccccc34)c3cccc4ccccc34)c3oc4ccccc4c23)cc1 ; c1ccc(-c2ccccc2N(c2ccccc2-c2ccc3ccccc3c2)c2ccc(N(c3ccccc3)c3ccc4ccccc4c3)c3c2oc2ccccc23)cc1] 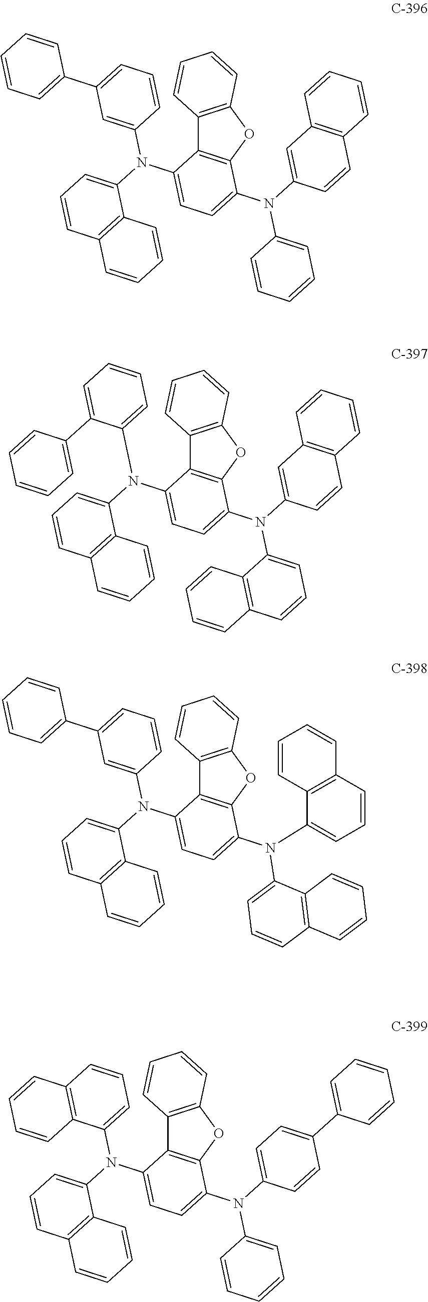 [SMILES: c1ccc(-c2cccc(N(c3cccc4ccccc34)c3ccc(N(c4ccccc4)c4ccc5ccccc5c4)c4oc5ccccc5c34)c2)cc1 ; c1ccc(-c2ccccc2N(c2cccc3ccccc23)c2ccc(N(c3ccc4ccccc4c3)c3cccc4ccccc34)c3oc4ccccc4c23)cc1 ; c1ccc(-c2cccc(N(c3cccc4ccccc34)c3ccc(N(c4cccc5ccccc45)c4cccc5ccccc45)c4oc5ccccc5c34)c2)cc1 ; c1ccc(-c2ccc(N(c3ccccc3)c3ccc(N(c4cccc5ccccc45)c4cccc5ccccc45)c4c3oc3ccccc34)cc2)cc1] 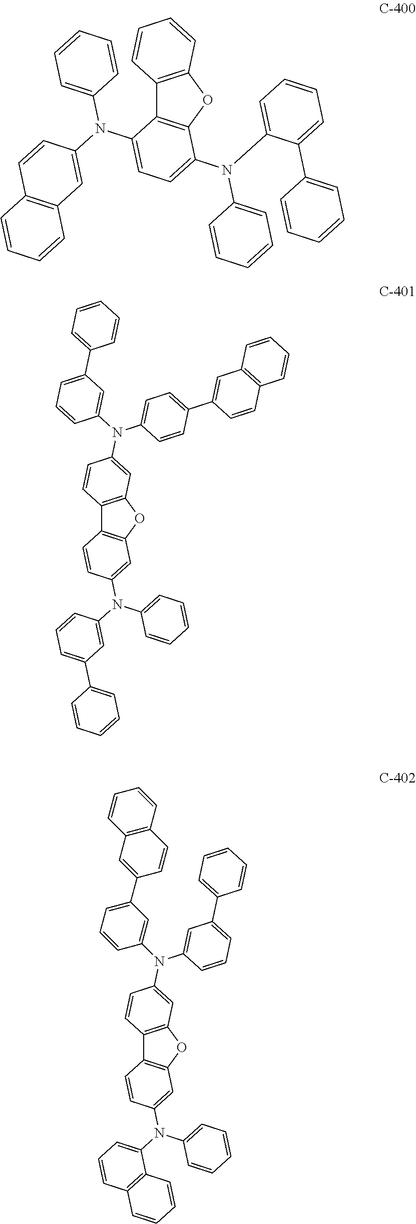 [SMILES: c1ccc(-c2ccccc2N(c2ccccc2)c2ccc(N(c3ccccc3)c3ccc4ccccc4c3)c3c2oc2ccccc23)cc1 ; c1ccc(-c2cccc(N(c3ccccc3)c3ccc4c(c3)oc3cc(N(c5ccc(-c6ccc7ccccc7c6)cc5)c5cccc(-c6ccccc6)c5)ccc34)c2)cc1 ; c1ccc(-c2cccc(N(c3cccc(-c4ccc5ccccc5c4)c3)c3ccc4c(c3)oc3cc(N(c5ccccc5)c5cccc6ccccc56)ccc34)c2)cc1] 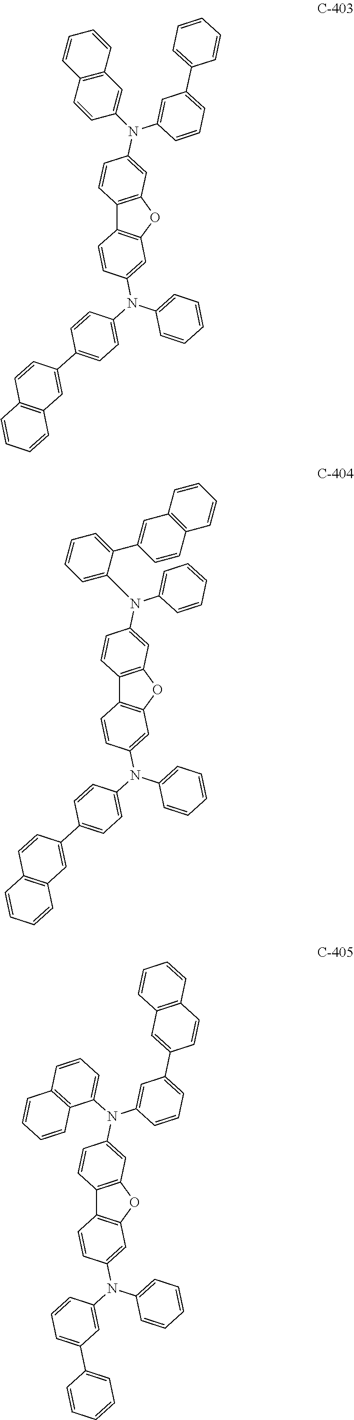 [SMILES: c1ccc(-c2cccc(N(c3ccc4ccccc4c3)c3ccc4c(c3)oc3cc(N(c5ccccc5)c5ccc(-c6ccc7ccccc7c6)cc5)ccc34)c2)cc1 ; c1ccc(N(c2ccc(-c3ccc4ccccc4c3)cc2)c2ccc3c(c2)oc2cc(N(c4ccccc4)c4ccccc4-c4ccc5ccccc5c4)ccc23)cc1 ; c1ccc(-c2cccc(N(c3ccccc3)c3ccc4c(c3)oc3cc(N(c5cccc(-c6ccc7ccccc7c6)c5)c5cccc6ccccc56)ccc34)c2)cc1] 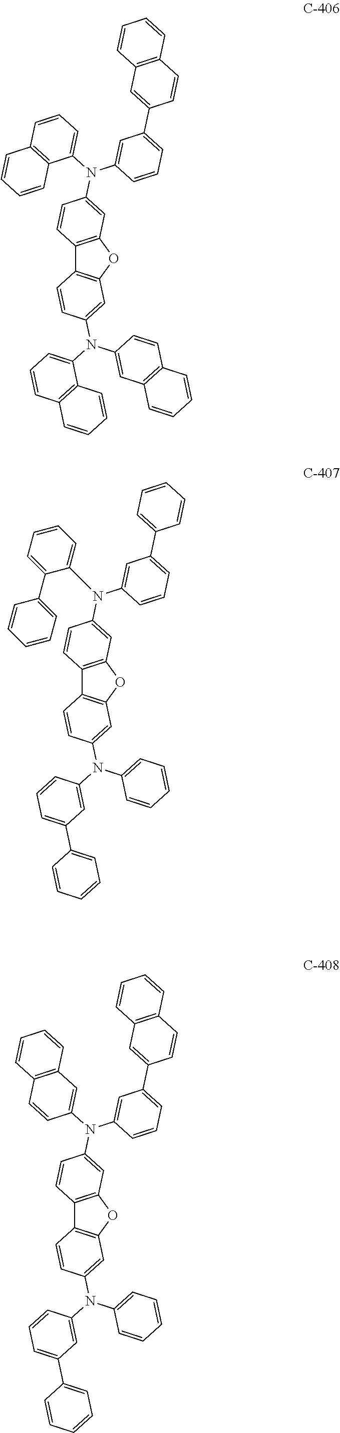 [SMILES: c1cc(-c2ccc3ccccc3c2)cc(N(c2ccc3c(c2)oc2cc(N(c4ccc5ccccc5c4)c4cccc5ccccc45)ccc23)c2cccc3ccccc23)c1 ; c1ccc(-c2cccc(N(c3ccccc3)c3ccc4c(c3)oc3cc(N(c5cccc(-c6ccccc6)c5)c5ccccc5-c5ccccc5)ccc34)c2)cc1 ; c1ccc(-c2cccc(N(c3ccccc3)c3ccc4c(c3)oc3cc(N(c5cccc(-c6ccc7ccccc7c6)c5)c5ccc6ccccc6c5)ccc34)c2)cc1] 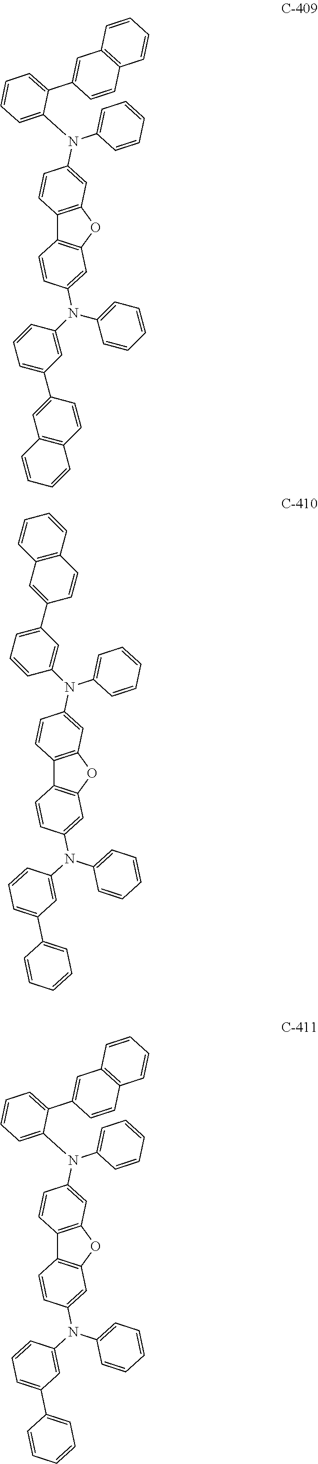 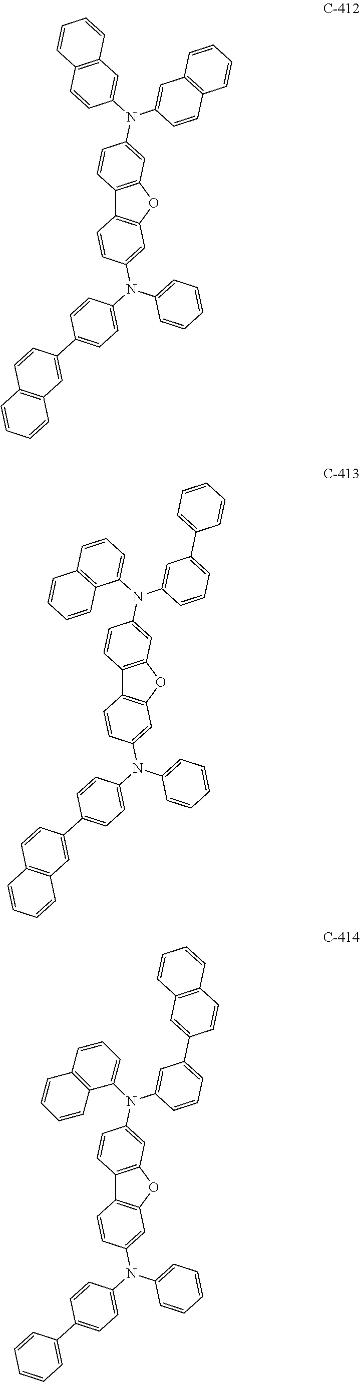 [SMILES: c1ccc(N(c2ccc(-c3ccc4ccccc4c3)cc2)c2ccc3c(c2)oc2cc(N(c4ccc5ccccc5c4)c4ccc5ccccc5c4)ccc23)cc1 ; c1ccc(-c2cccc(N(c3ccc4c(c3)oc3cc(N(c5ccccc5)c5ccc(-c6ccc7ccccc7c6)cc5)ccc34)c3cccc4ccccc34)c2)cc1 ; c1ccc(-c2ccc(N(c3ccccc3)c3ccc4c(c3)oc3cc(N(c5cccc(-c6ccc7ccccc7c6)c5)c5cccc6ccccc56)ccc34)cc2)cc1] 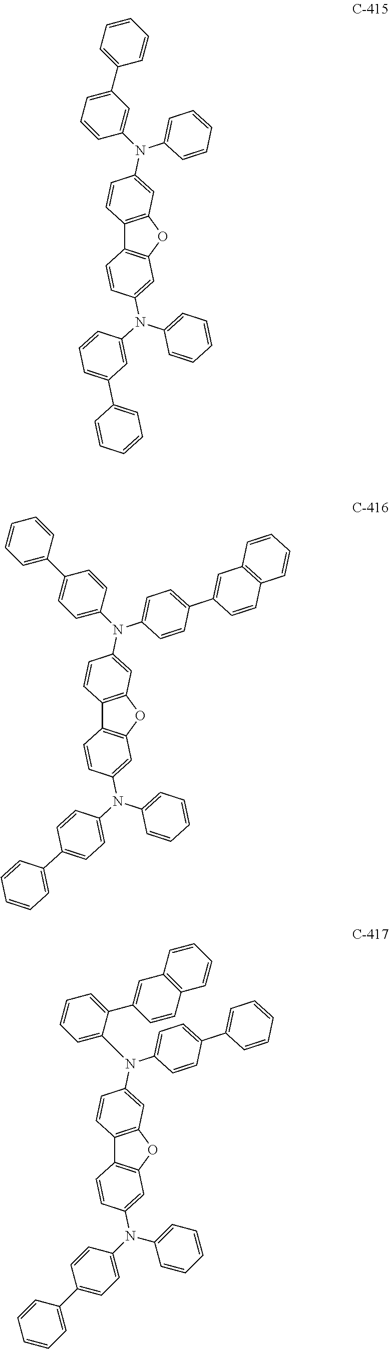 [SMILES: c1ccc(-c2cccc(N(c3ccccc3)c3ccc4c(c3)oc3cc(N(c5ccccc5)c5cccc(-c6ccccc6)c5)ccc34)c2)cc1 ; c1ccc(-c2ccc(N(c3ccccc3)c3ccc4c(c3)oc3cc(N(c5ccc(-c6ccccc6)cc5)c5ccc(-c6ccc7ccccc7c6)cc5)ccc34)cc2)cc1 ; c1ccc(-c2ccc(N(c3ccccc3)c3ccc4c(c3)oc3cc(N(c5ccc(-c6ccccc6)cc5)c5ccccc5-c5ccc6ccccc6c5)ccc34)cc2)cc1] 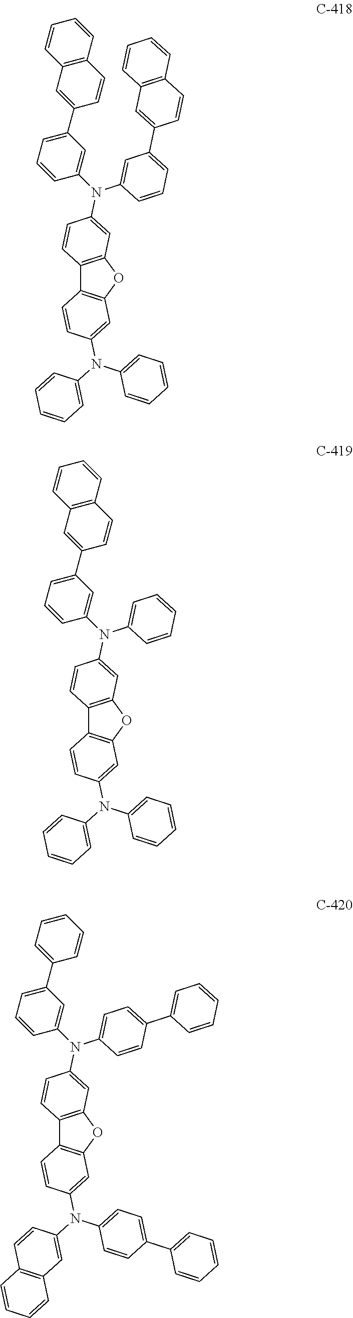 [SMILES: c1ccc(N(c2ccccc2)c2ccc3c(c2)oc2cc(N(c4cccc(-c5ccc6ccccc6c5)c4)c4cccc(-c5ccc6ccccc6c5)c4)ccc23)cc1 ; c1ccc(N(c2ccccc2)c2ccc3c(c2)oc2cc(N(c4ccccc4)c4cccc(-c5ccc6ccccc6c5)c4)ccc23)cc1 ; c1ccc(-c2ccc(N(c3cccc(-c4ccccc4)c3)c3ccc4c(c3)oc3cc(N(c5ccc(-c6ccccc6)cc5)c5ccc6ccccc6c5)ccc34)cc2)cc1] 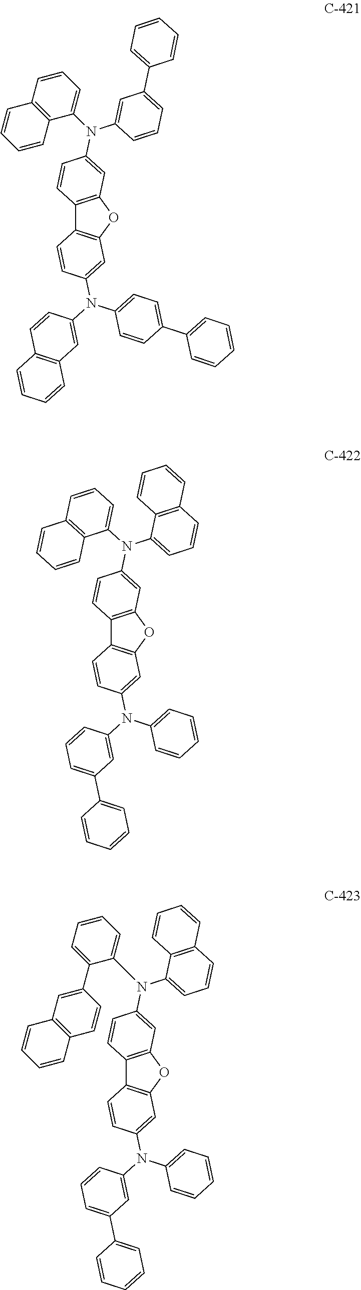 [SMILES: c1ccc(-c2ccc(N(c3ccc4ccccc4c3)c3ccc4c(c3)oc3cc(N(c5cccc(-c6ccccc6)c5)c5cccc6ccccc56)ccc34)cc2)cc1 ; c1ccc(-c2cccc(N(c3ccccc3)c3ccc4c(c3)oc3cc(N(c5cccc6ccccc56)c5cccc6ccccc56)ccc34)c2)cc1 ; c1ccc(-c2cccc(N(c3ccccc3)c3ccc4c(c3)oc3cc(N(c5ccccc5-c5ccc6ccccc6c5)c5cccc6ccccc56)ccc34)c2)cc1] 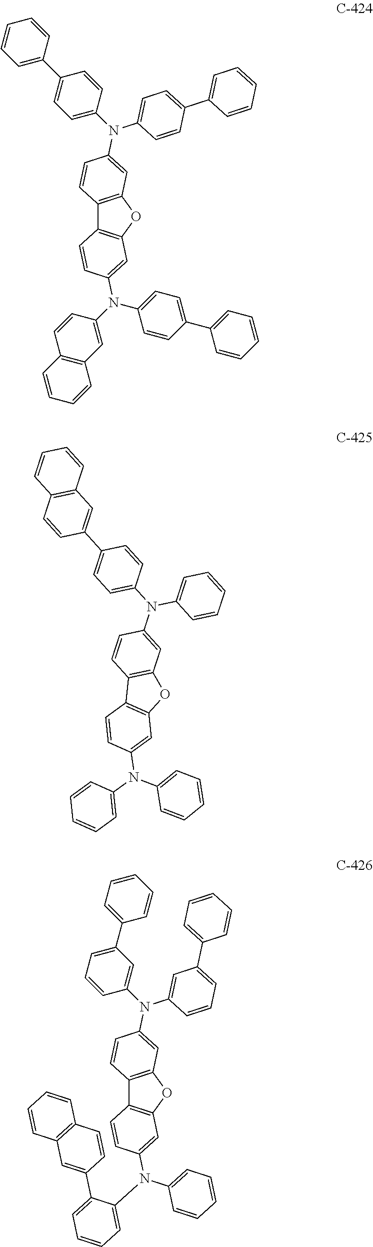 [SMILES: c1ccc(-c2ccc(N(c3ccc(-c4ccccc4)cc3)c3ccc4c(c3)oc3cc(N(c5ccc(-c6ccccc6)cc5)c5ccc6ccccc6c5)ccc34)cc2)cc1 ; c1ccc(N(c2ccccc2)c2ccc3c(c2)oc2cc(N(c4ccccc4)c4ccc(-c5ccc6ccccc6c5)cc4)ccc23)cc1 ; c1ccc(-c2cccc(N(c3cccc(-c4ccccc4)c3)c3ccc4c(c3)oc3cc(N(c5ccccc5)c5ccccc5-c5ccc6ccccc6c5)ccc34)c2)cc1] 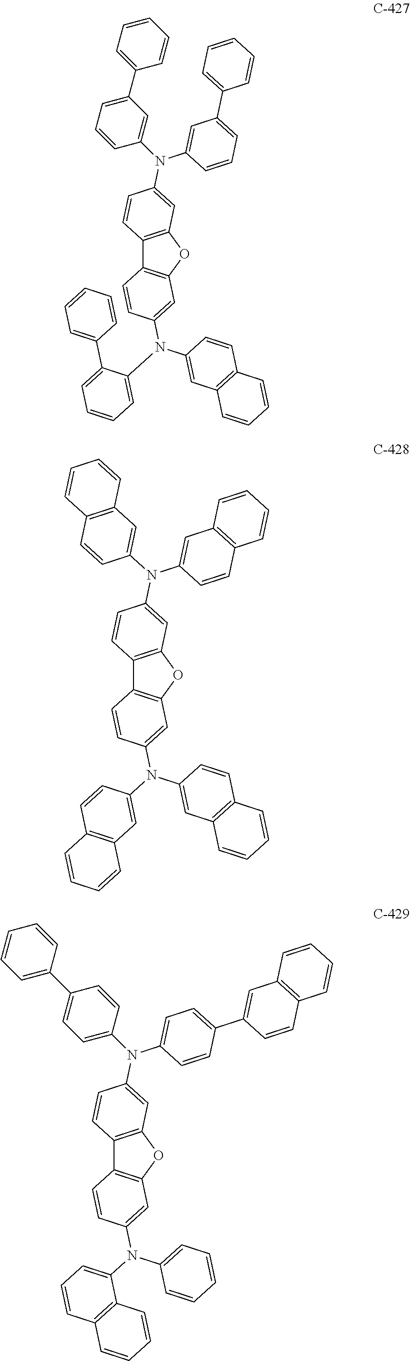 [SMILES: c1ccc(-c2cccc(N(c3cccc(-c4ccccc4)c3)c3ccc4c(c3)oc3cc(N(c5ccc6ccccc6c5)c5ccccc5-c5ccccc5)ccc34)c2)cc1 ; c1ccc2cc(N(c3ccc4ccccc4c3)c3ccc4c(c3)oc3cc(N(c5ccc6ccccc6c5)c5ccc6ccccc6c5)ccc34)ccc2c1 ; c1ccc(-c2ccc(N(c3ccc(-c4ccc5ccccc5c4)cc3)c3ccc4c(c3)oc3cc(N(c5ccccc5)c5cccc6ccccc56)ccc34)cc2)cc1] 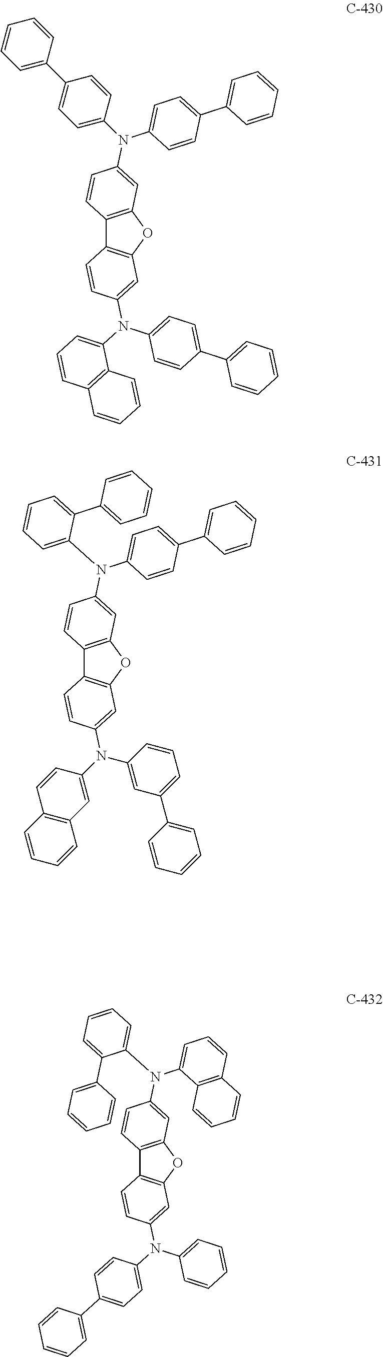 [SMILES: c1ccc(-c2ccc(N(c3ccc(-c4ccccc4)cc3)c3ccc4c(c3)oc3cc(N(c5ccc(-c6ccccc6)cc5)c5cccc6ccccc56)ccc34)cc2)cc1 ; c1ccc(-c2ccc(N(c3ccc4c(c3)oc3cc(N(c5cccc(-c6ccccc6)c5)c5ccc6ccccc6c5)ccc34)c3ccccc3-c3ccccc3)cc2)cc1 ; c1ccc(-c2ccc(N(c3ccccc3)c3ccc4c(c3)oc3cc(N(c5ccccc5-c5ccccc5)c5cccc6ccccc56)ccc34)cc2)cc1] 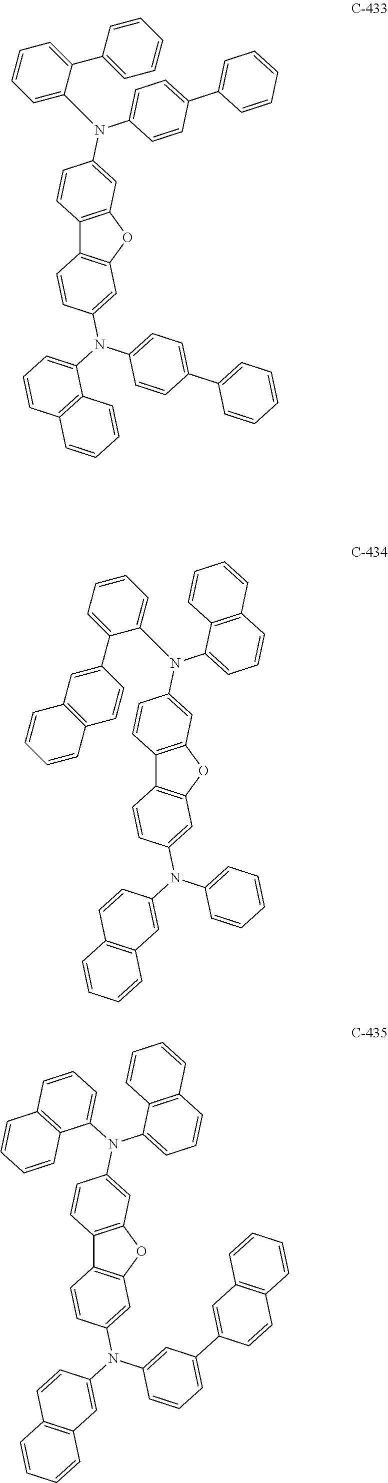 [SMILES: c1ccc(-c2ccc(N(c3ccc4c(c3)oc3cc(N(c5ccc(-c6ccccc6)cc5)c5cccc6ccccc56)ccc34)c3ccccc3-c3ccccc3)cc2)cc1 ; c1ccc(N(c2ccc3ccccc3c2)c2ccc3c(c2)oc2cc(N(c4ccccc4-c4ccc5ccccc5c4)c4cccc5ccccc45)ccc23)cc1 ; c1cc(-c2ccc3ccccc3c2)cc(N(c2ccc3ccccc3c2)c2ccc3c(c2)oc2cc(N(c4cccc5ccccc45)c4cccc5ccccc45)ccc23)c1] 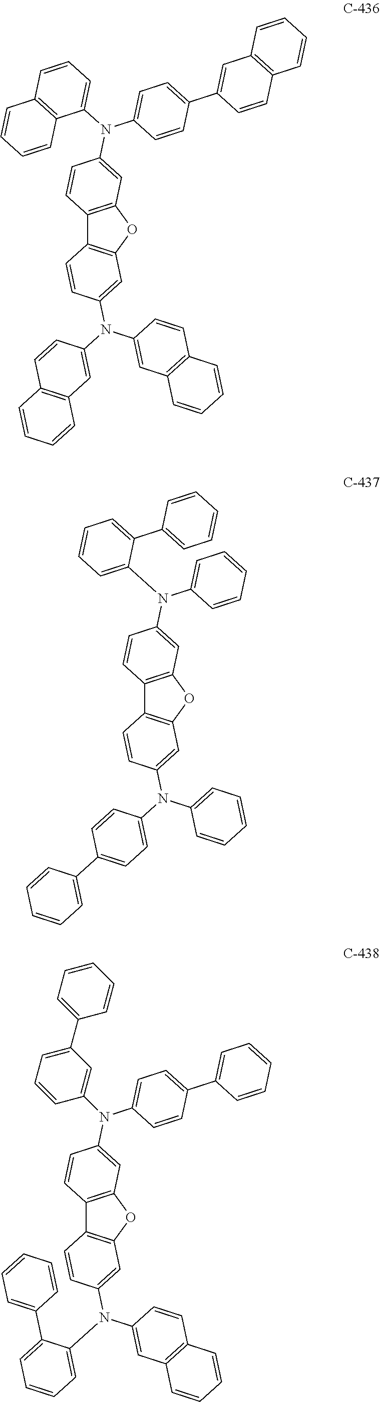 [SMILES: c1ccc2cc(-c3ccc(N(c4ccc5c(c4)oc4cc(N(c6ccc7ccccc7c6)c6ccc7ccccc7c6)ccc45)c4cccc5ccccc45)cc3)ccc2c1 ; c1ccc(-c2ccc(N(c3ccccc3)c3ccc4c(c3)oc3cc(N(c5ccccc5)c5ccccc5-c5ccccc5)ccc34)cc2)cc1 ; c1ccc(-c2ccc(N(c3cccc(-c4ccccc4)c3)c3ccc4c(c3)oc3cc(N(c5ccc6ccccc6c5)c5ccccc5-c5ccccc5)ccc34)cc2)cc1] 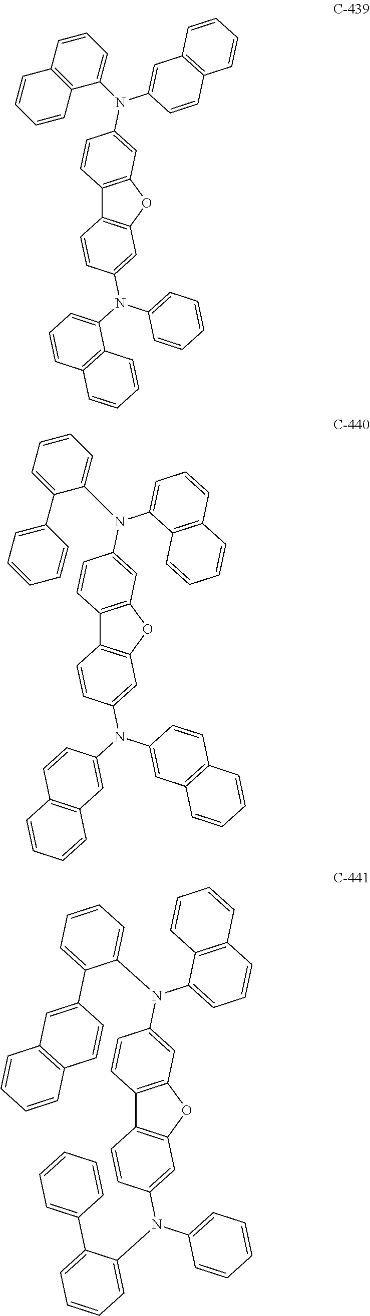 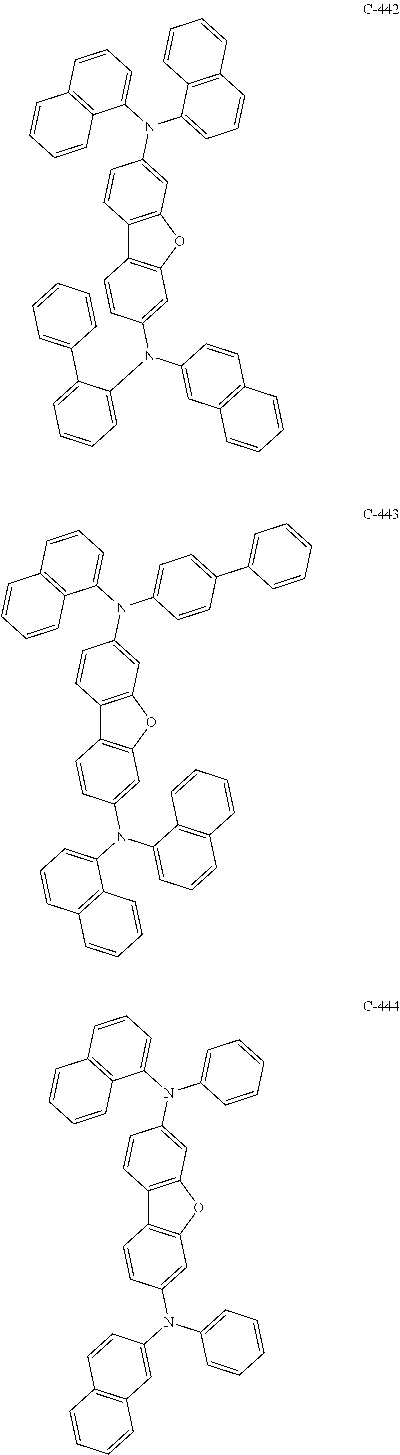 [SMILES: c1ccc(-c2ccccc2N(c2ccc3ccccc3c2)c2ccc3c(c2)oc2cc(N(c4cccc5ccccc45)c4cccc5ccccc45)ccc23)cc1 ; c1ccc(-c2ccc(N(c3ccc4c(c3)oc3cc(N(c5cccc6ccccc56)c5cccc6ccccc56)ccc34)c3cccc4ccccc34)cc2)cc1 ; c1ccc(N(c2ccc3ccccc3c2)c2ccc3c(c2)oc2cc(N(c4ccccc4)c4cccc5ccccc45)ccc23)cc1] 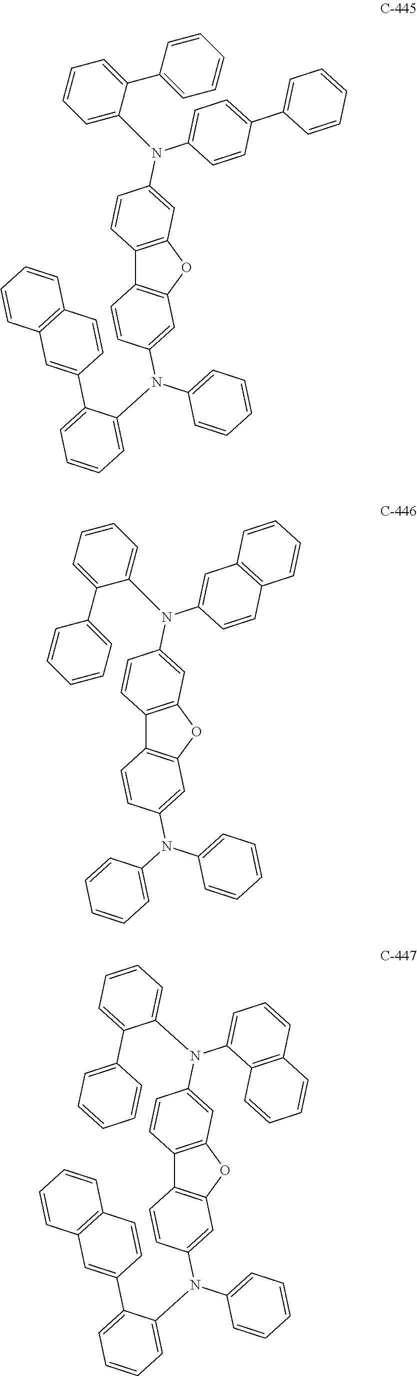 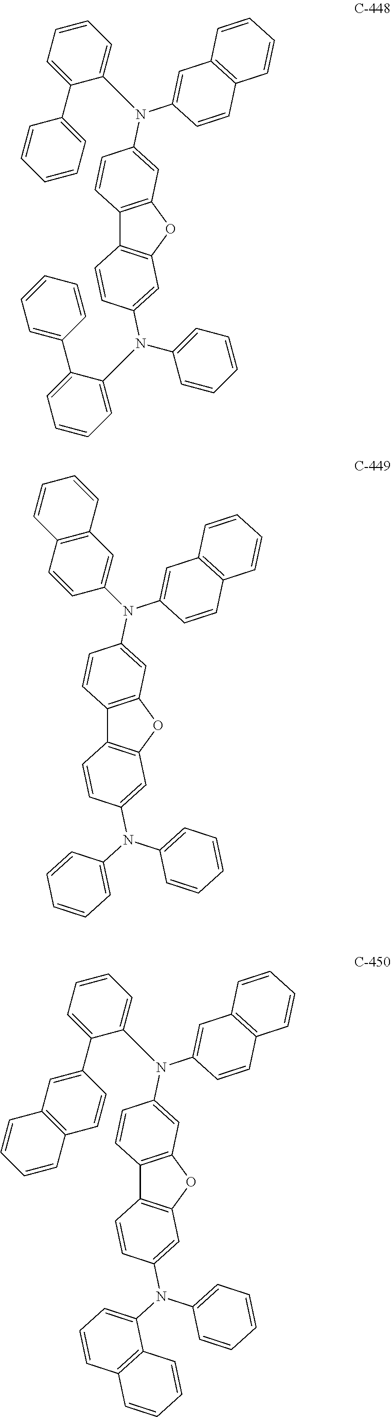 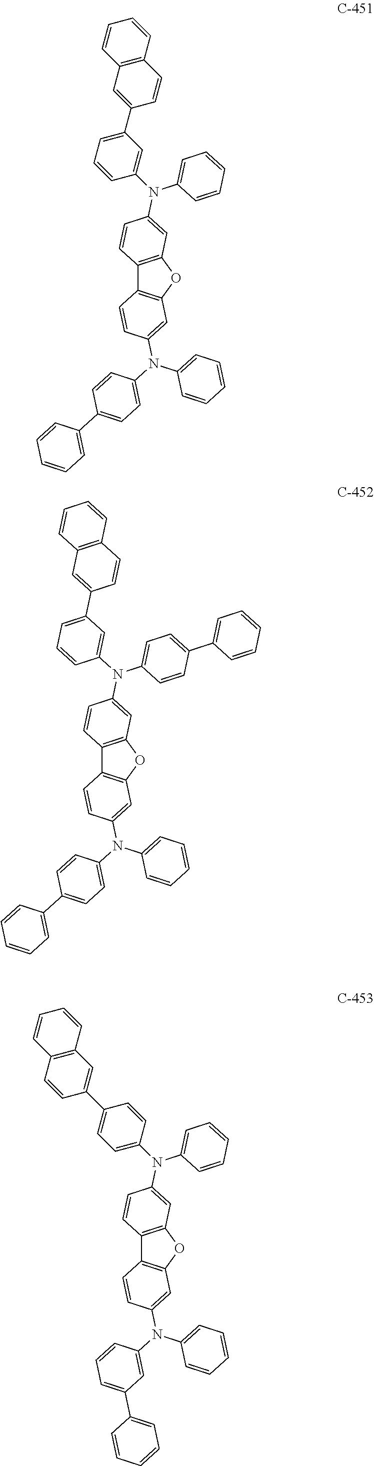 [SMILES: c1ccc(-c2ccc(N(c3ccccc3)c3ccc4c(c3)oc3cc(N(c5ccccc5)c5cccc(-c6ccc7ccccc7c6)c5)ccc34)cc2)cc1 ; c1ccc(-c2ccc(N(c3ccccc3)c3ccc4c(c3)oc3cc(N(c5ccc(-c6ccccc6)cc5)c5cccc(-c6ccc7ccccc7c6)c5)ccc34)cc2)cc1 ; c1ccc(-c2cccc(N(c3ccccc3)c3ccc4c(c3)oc3cc(N(c5ccccc5)c5ccc(-c6ccc7ccccc7c6)cc5)ccc34)c2)cc1] 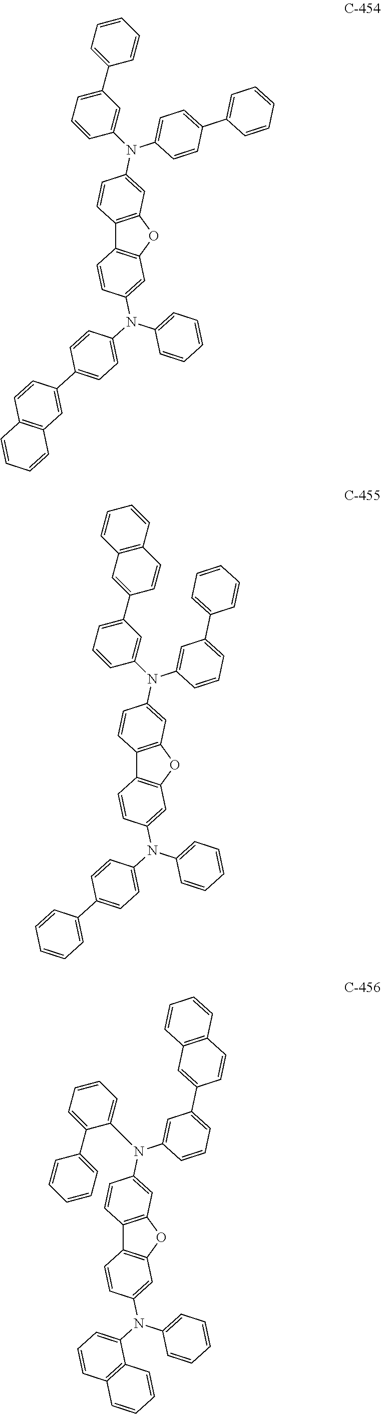 [SMILES: c1ccc(-c2ccc(N(c3cccc(-c4ccccc4)c3)c3ccc4c(c3)oc3cc(N(c5ccccc5)c5ccc(-c6ccc7ccccc7c6)cc5)ccc34)cc2)cc1 ; c1ccc(-c2ccc(N(c3ccccc3)c3ccc4c(c3)oc3cc(N(c5cccc(-c6ccccc6)c5)c5cccc(-c6ccc7ccccc7c6)c5)ccc34)cc2)cc1 ; c1ccc(-c2ccccc2N(c2cccc(-c3ccc4ccccc4c3)c2)c2ccc3c(c2)oc2cc(N(c4ccccc4)c4cccc5ccccc45)ccc23)cc1] 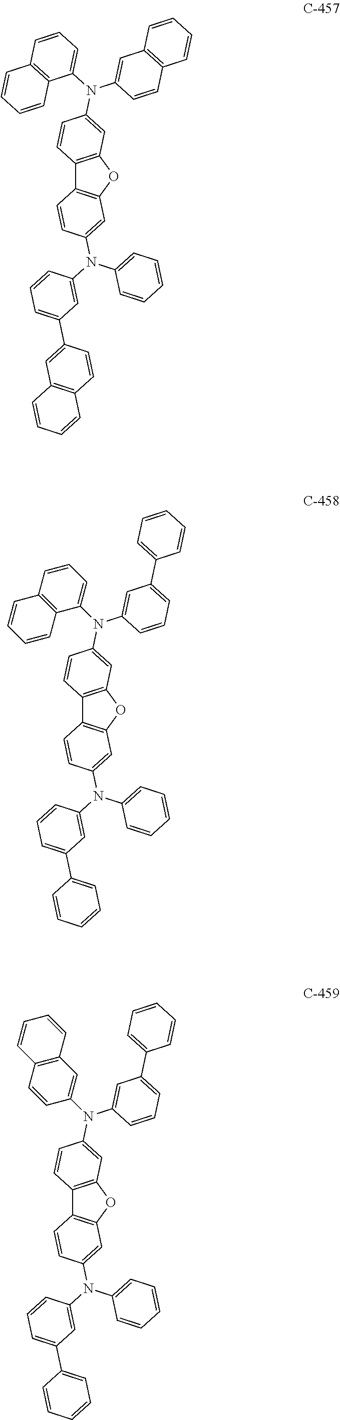 [SMILES: c1ccc(N(c2cccc(-c3ccc4ccccc4c3)c2)c2ccc3c(c2)oc2cc(N(c4ccc5ccccc5c4)c4cccc5ccccc45)ccc23)cc1 ; c1ccc(-c2cccc(N(c3ccccc3)c3ccc4c(c3)oc3cc(N(c5cccc(-c6ccccc6)c5)c5cccc6ccccc56)ccc34)c2)cc1 ; c1ccc(-c2cccc(N(c3ccccc3)c3ccc4c(c3)oc3cc(N(c5cccc(-c6ccccc6)c5)c5ccc6ccccc6c5)ccc34)c2)cc1] 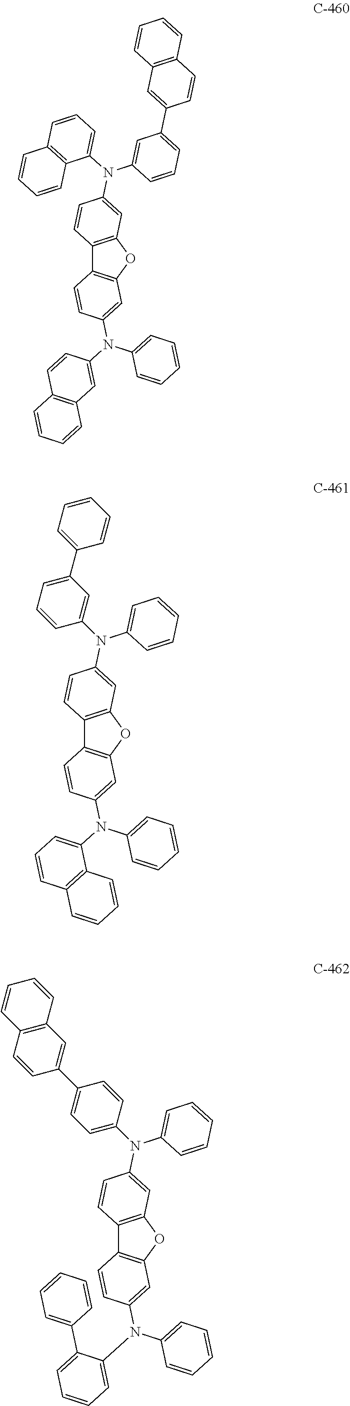 [SMILES: c1ccc(N(c2ccc3ccccc3c2)c2ccc3c(c2)oc2cc(N(c4cccc(-c5ccc6ccccc6c5)c4)c4cccc5ccccc45)ccc23)cc1 ; c1ccc(-c2cccc(N(c3ccccc3)c3ccc4c(c3)oc3cc(N(c5ccccc5)c5cccc6ccccc56)ccc34)c2)cc1 ; c1ccc(-c2ccccc2N(c2ccccc2)c2ccc3c(c2)oc2cc(N(c4ccccc4)c4ccc(-c5ccc6ccccc6c5)cc4)ccc23)cc1] 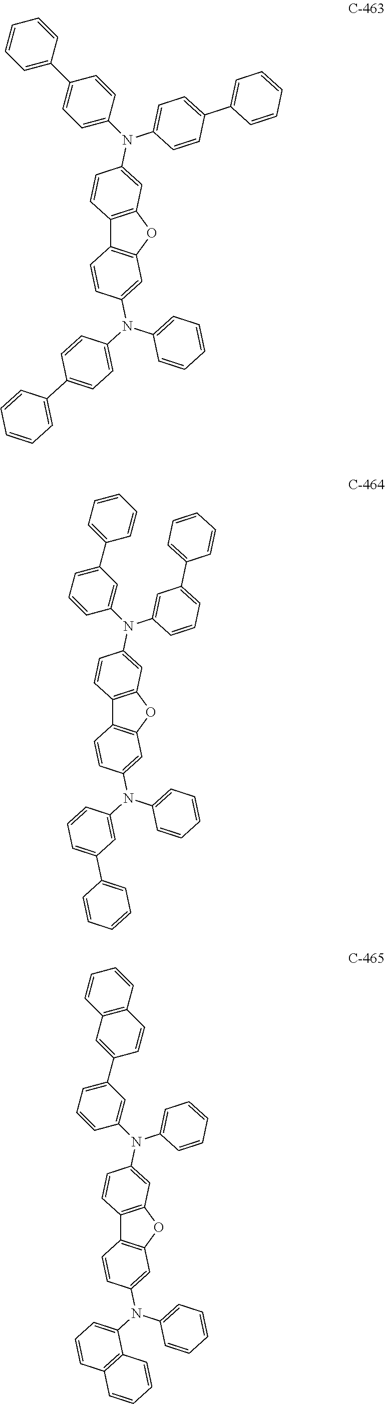 [SMILES: c1ccc(-c2ccc(N(c3ccccc3)c3ccc4c(c3)oc3cc(N(c5ccc(-c6ccccc6)cc5)c5ccc(-c6ccccc6)cc5)ccc34)cc2)cc1 ; c1ccc(-c2cccc(N(c3ccccc3)c3ccc4c(c3)oc3cc(N(c5cccc(-c6ccccc6)c5)c5cccc(-c6ccccc6)c5)ccc34)c2)cc1 ; c1ccc(N(c2cccc(-c3ccc4ccccc4c3)c2)c2ccc3c(c2)oc2cc(N(c4ccccc4)c4cccc5ccccc45)ccc23)cc1] 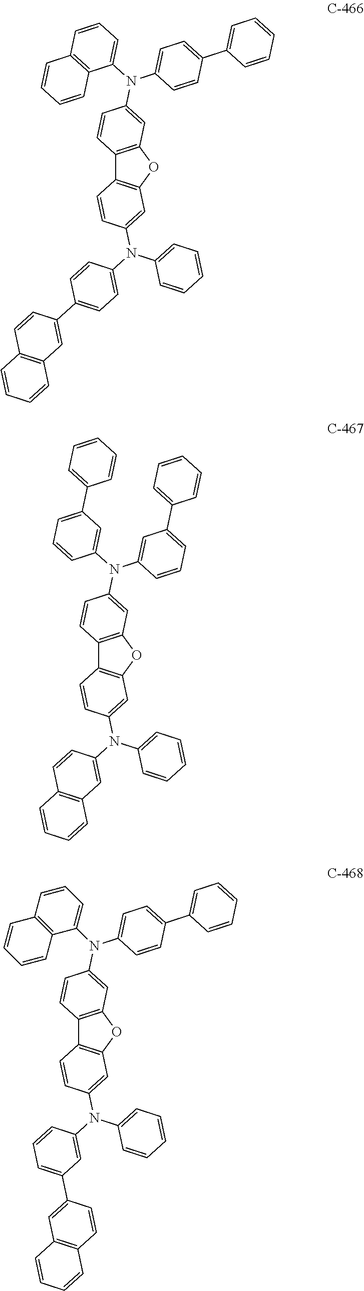 [SMILES: c1ccc(-c2ccc(N(c3ccc4c(c3)oc3cc(N(c5ccccc5)c5ccc(-c6ccc7ccccc7c6)cc5)ccc34)c3cccc4ccccc34)cc2)cc1 ; c1ccc(-c2cccc(N(c3cccc(-c4ccccc4)c3)c3ccc4c(c3)oc3cc(N(c5ccccc5)c5ccc6ccccc6c5)ccc34)c2)cc1 ; c1ccc(-c2ccc(N(c3ccc4c(c3)oc3cc(N(c5ccccc5)c5cccc(-c6ccc7ccccc7c6)c5)ccc34)c3cccc4ccccc34)cc2)cc1] 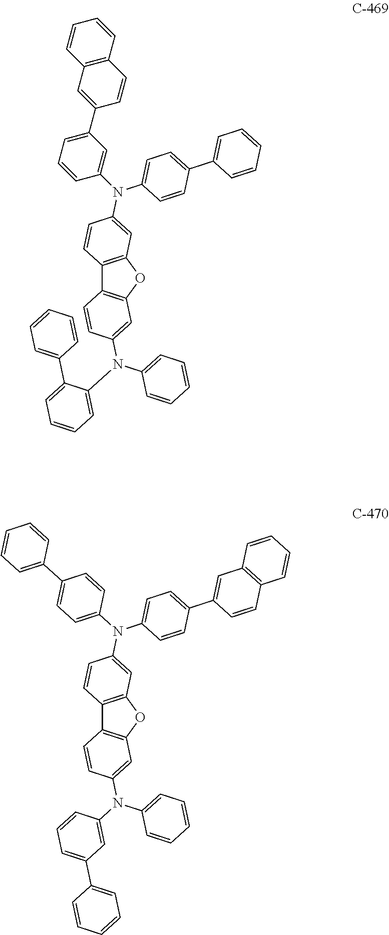 [SMILES: c1ccc(-c2ccc(N(c3cccc(-c4ccc5ccccc5c4)c3)c3ccc4c(c3)oc3cc(N(c5ccccc5)c5ccccc5-c5ccccc5)ccc34)cc2)cc1 ; c1ccc(-c2ccc(N(c3ccc(-c4ccc5ccccc5c4)cc3)c3ccc4c(c3)oc3cc(N(c5ccccc5)c5cccc(-c6ccccc6)c5)ccc34)cc2)cc1] 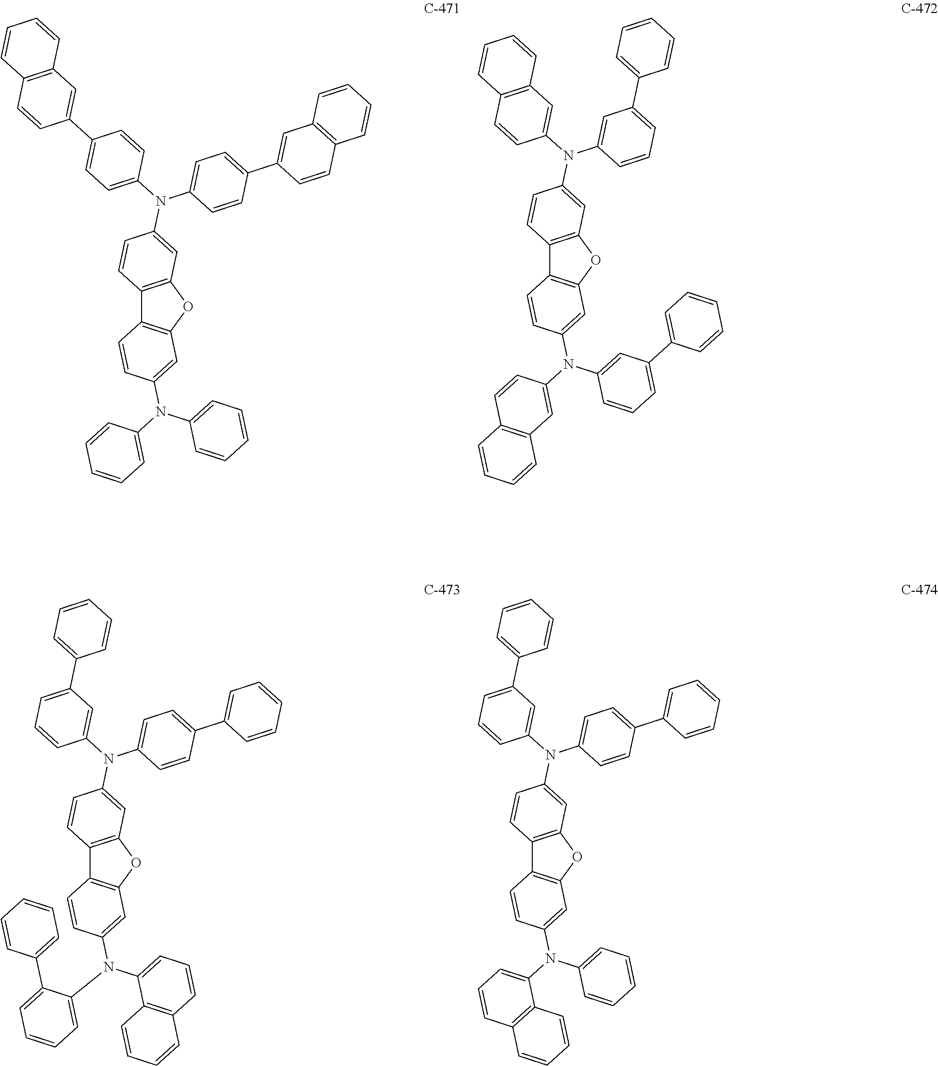 [SMILES: c1ccc(N(c2ccccc2)c2ccc3c(c2)oc2cc(N(c4ccc(-c5ccc6ccccc6c5)cc4)c4ccc(-c5ccc6ccccc6c5)cc4)ccc23)cc1 ; c1ccc(-c2cccc(N(c3ccc4ccccc4c3)c3ccc4c(c3)oc3cc(N(c5cccc(-c6ccccc6)c5)c5ccc6ccccc6c5)ccc34)c2)cc1 ; c1ccc(-c2ccc(N(c3cccc(-c4ccccc4)c3)c3ccc4c(c3)oc3cc(N(c5ccccc5-c5ccccc5)c5cccc6ccccc56)ccc34)cc2)cc1 ; c1ccc(-c2ccc(N(c3cccc(-c4ccccc4)c3)c3ccc4c(c3)oc3cc(N(c5ccccc5)c5cccc6ccccc56)ccc34)cc2)cc1] 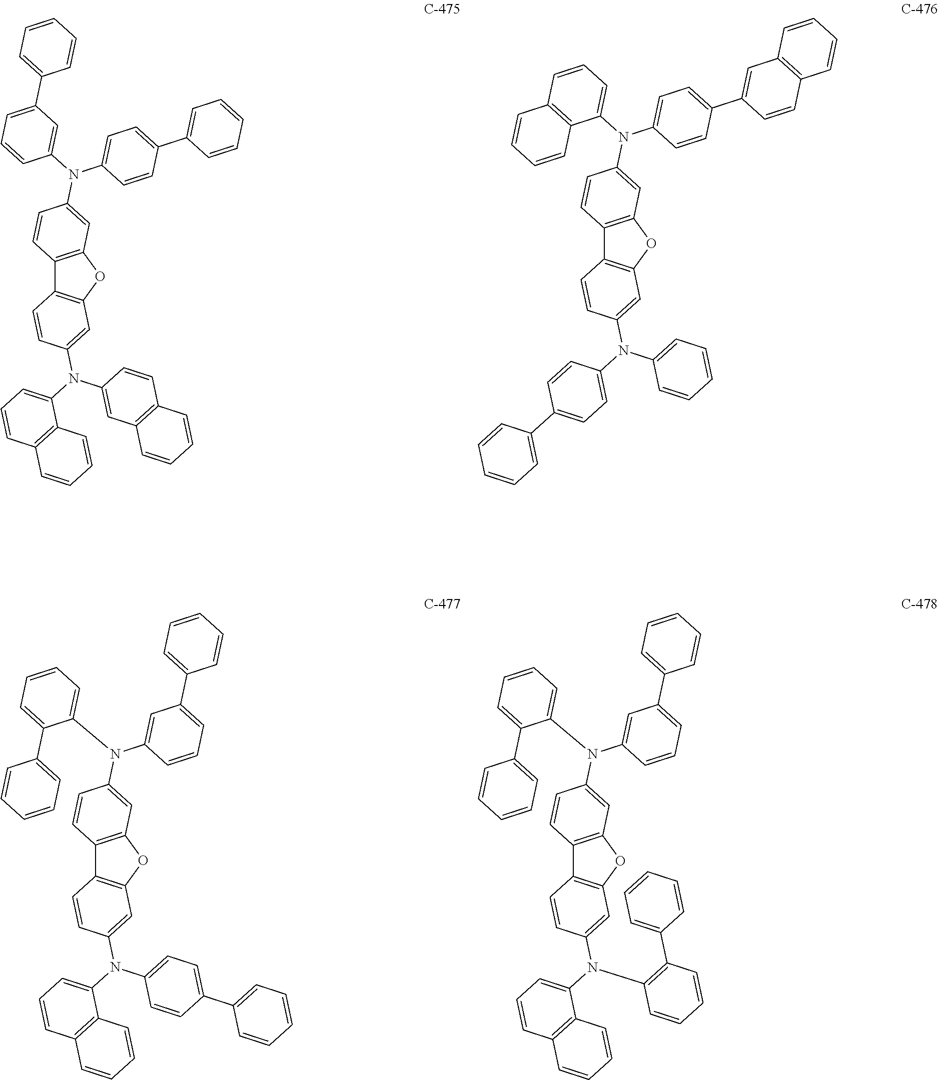 [SMILES: c1ccc(-c2ccc(N(c3cccc(-c4ccccc4)c3)c3ccc4c(c3)oc3cc(N(c5ccc6ccccc6c5)c5cccc6ccccc56)ccc34)cc2)cc1 ; c1ccc(-c2ccc(N(c3ccccc3)c3ccc4c(c3)oc3cc(N(c5ccc(-c6ccc7ccccc7c6)cc5)c5cccc6ccccc56)ccc34)cc2)cc1 ; c1ccc(-c2ccc(N(c3ccc4c(c3)oc3cc(N(c5cccc(-c6ccccc6)c5)c5ccccc5-c5ccccc5)ccc34)c3cccc4ccccc34)cc2)cc1 ; c1ccc(-c2cccc(N(c3ccc4c(c3)oc3cc(N(c5ccccc5-c5ccccc5)c5cccc6ccccc56)ccc34)c3ccccc3-c3ccccc3)c2)cc1] 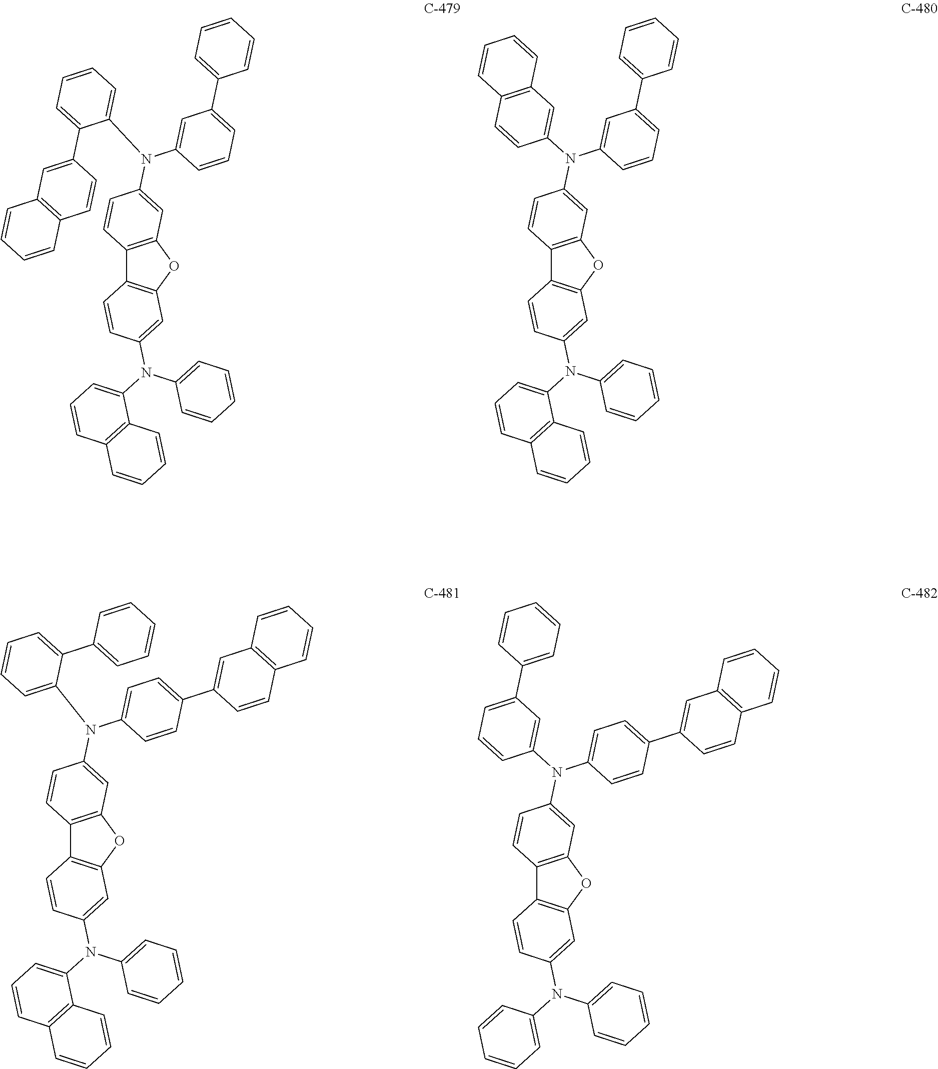 [SMILES: c1ccc(-c2cccc(N(c3ccc4c(c3)oc3cc(N(c5ccccc5)c5cccc6ccccc56)ccc34)c3ccccc3-c3ccc4ccccc4c3)c2)cc1 ; c1ccc(-c2cccc(N(c3ccc4ccccc4c3)c3ccc4c(c3)oc3cc(N(c5ccccc5)c5cccc6ccccc56)ccc34)c2)cc1 ; c1ccc(-c2ccccc2N(c2ccc(-c3ccc4ccccc4c3)cc2)c2ccc3c(c2)oc2cc(N(c4ccccc4)c4cccc5ccccc45)ccc23)cc1 ; c1ccc(-c2cccc(N(c3ccc(-c4ccc5ccccc5c4)cc3)c3ccc4c(c3)oc3cc(N(c5ccccc5)c5ccccc5)ccc34)c2)cc1] 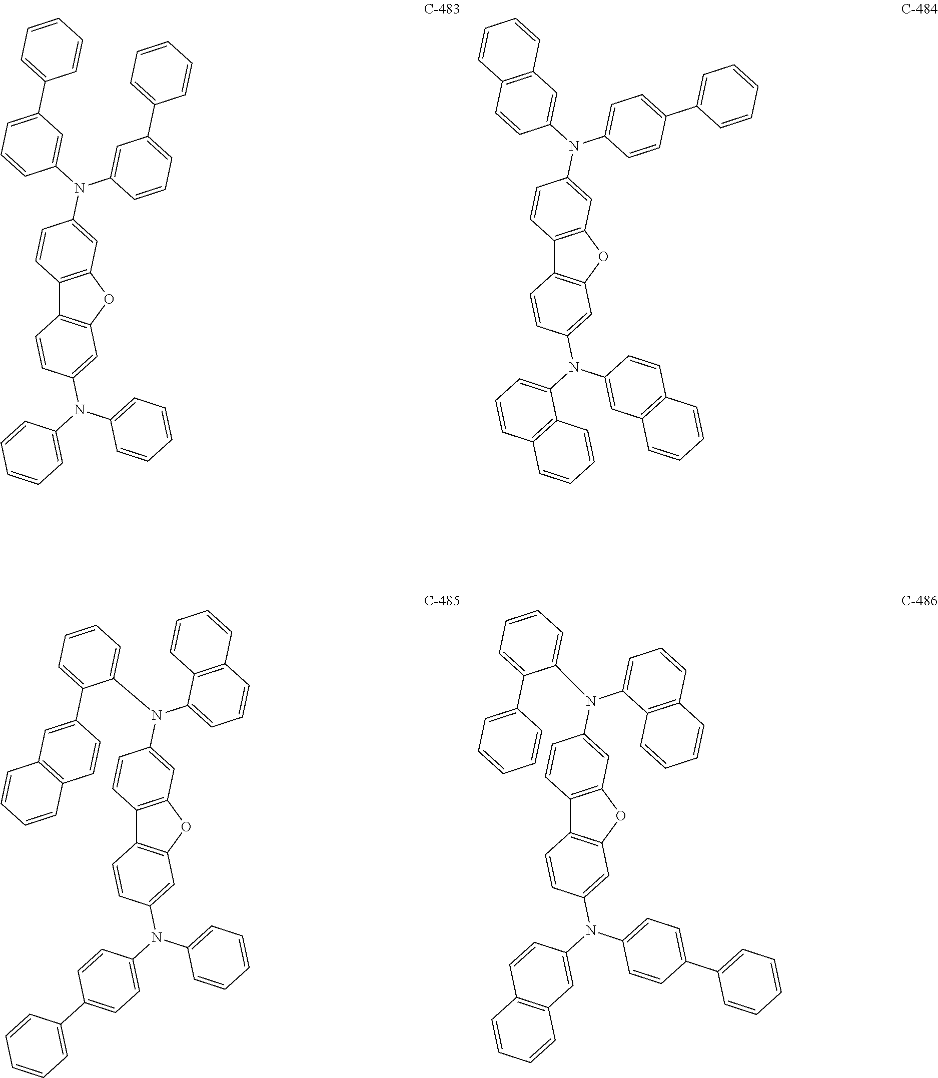 [SMILES: c1ccc(-c2cccc(N(c3cccc(-c4ccccc4)c3)c3ccc4c(c3)oc3cc(N(c5ccccc5)c5ccccc5)ccc34)c2)cc1 ; c1ccc(-c2ccc(N(c3ccc4ccccc4c3)c3ccc4c(c3)oc3cc(N(c5ccc6ccccc6c5)c5cccc6ccccc56)ccc34)cc2)cc1 ; c1ccc(-c2ccc(N(c3ccccc3)c3ccc4c(c3)oc3cc(N(c5ccccc5-c5ccc6ccccc6c5)c5cccc6ccccc56)ccc34)cc2)cc1 ; c1ccc(-c2ccc(N(c3ccc4ccccc4c3)c3ccc4c(c3)oc3cc(N(c5ccccc5-c5ccccc5)c5cccc6ccccc56)ccc34)cc2)cc1] 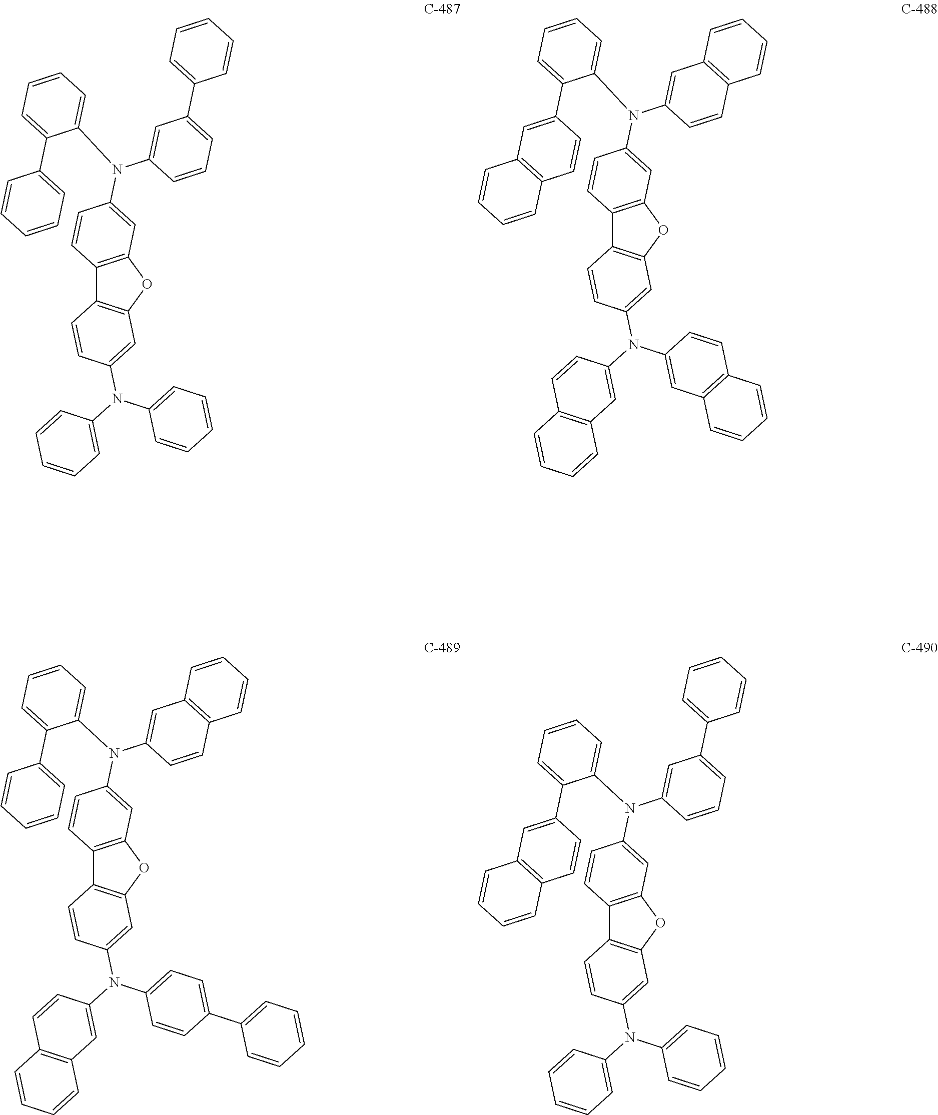 [SMILES: c1ccc(-c2cccc(N(c3ccc4c(c3)oc3cc(N(c5ccccc5)c5ccccc5)ccc34)c3ccccc3-c3ccccc3)c2)cc1 ; c1ccc(N(c2ccc3ccccc3c2)c2ccc3c(c2)oc2cc(N(c4ccc5ccccc5c4)c4ccc5ccccc5c4)ccc23)c(-c2ccc3ccccc3c2)c1 ; c1ccc(-c2ccc(N(c3ccc4ccccc4c3)c3ccc4c(c3)oc3cc(N(c5ccc6ccccc6c5)c5ccccc5-c5ccccc5)ccc34)cc2)cc1 ; c1ccc(-c2cccc(N(c3ccc4c(c3)oc3cc(N(c5ccccc5)c5ccccc5)ccc34)c3ccccc3-c3ccc4ccccc4c3)c2)cc1] 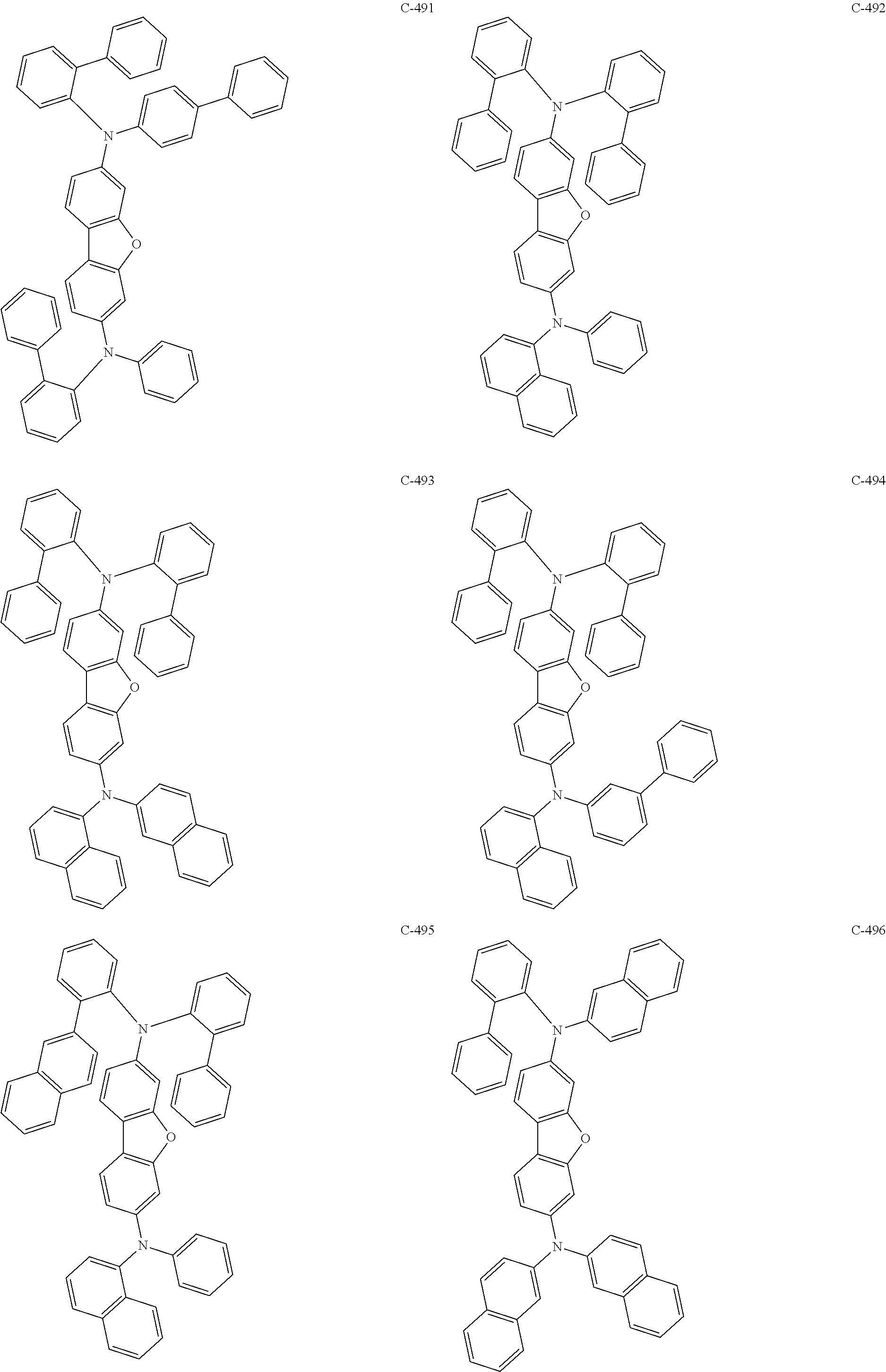 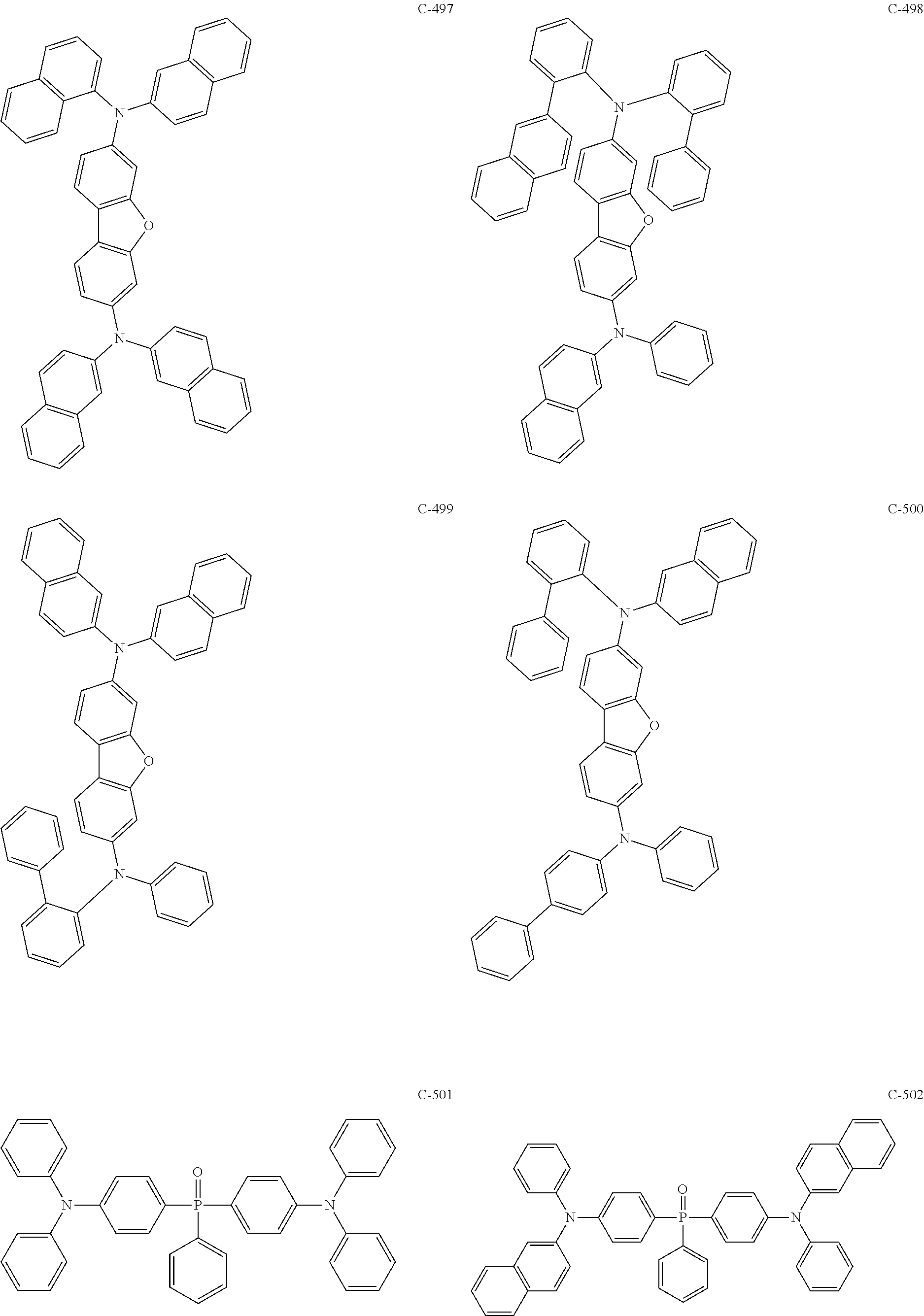 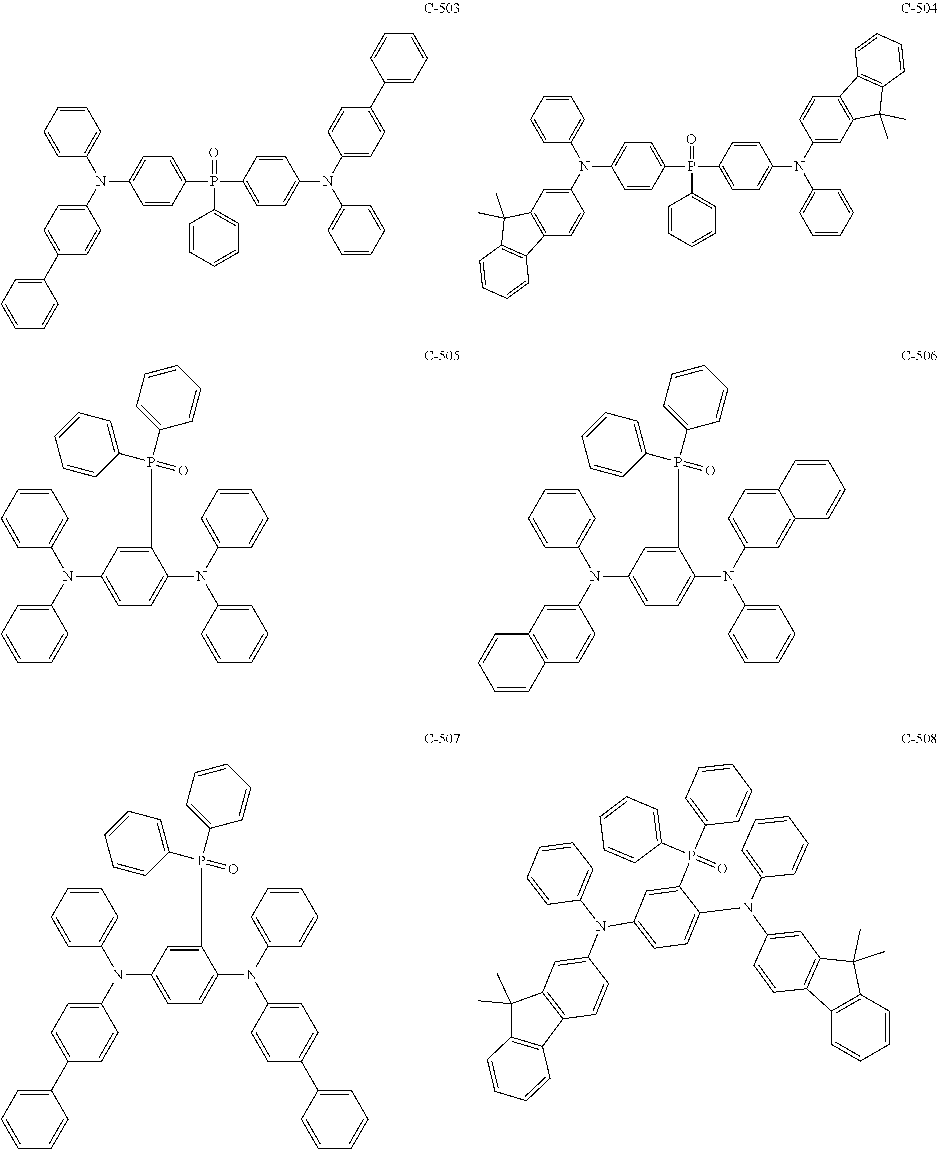 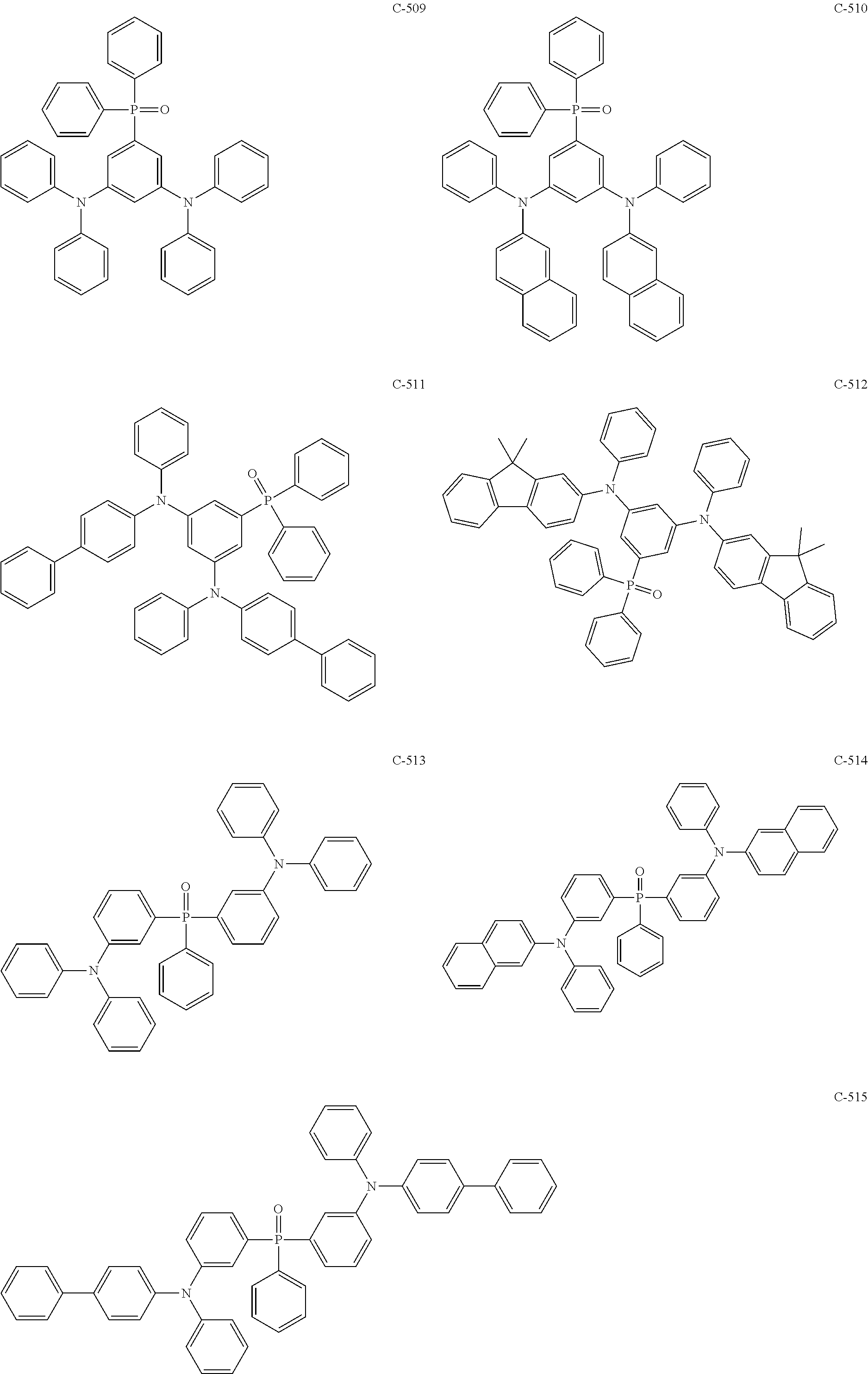 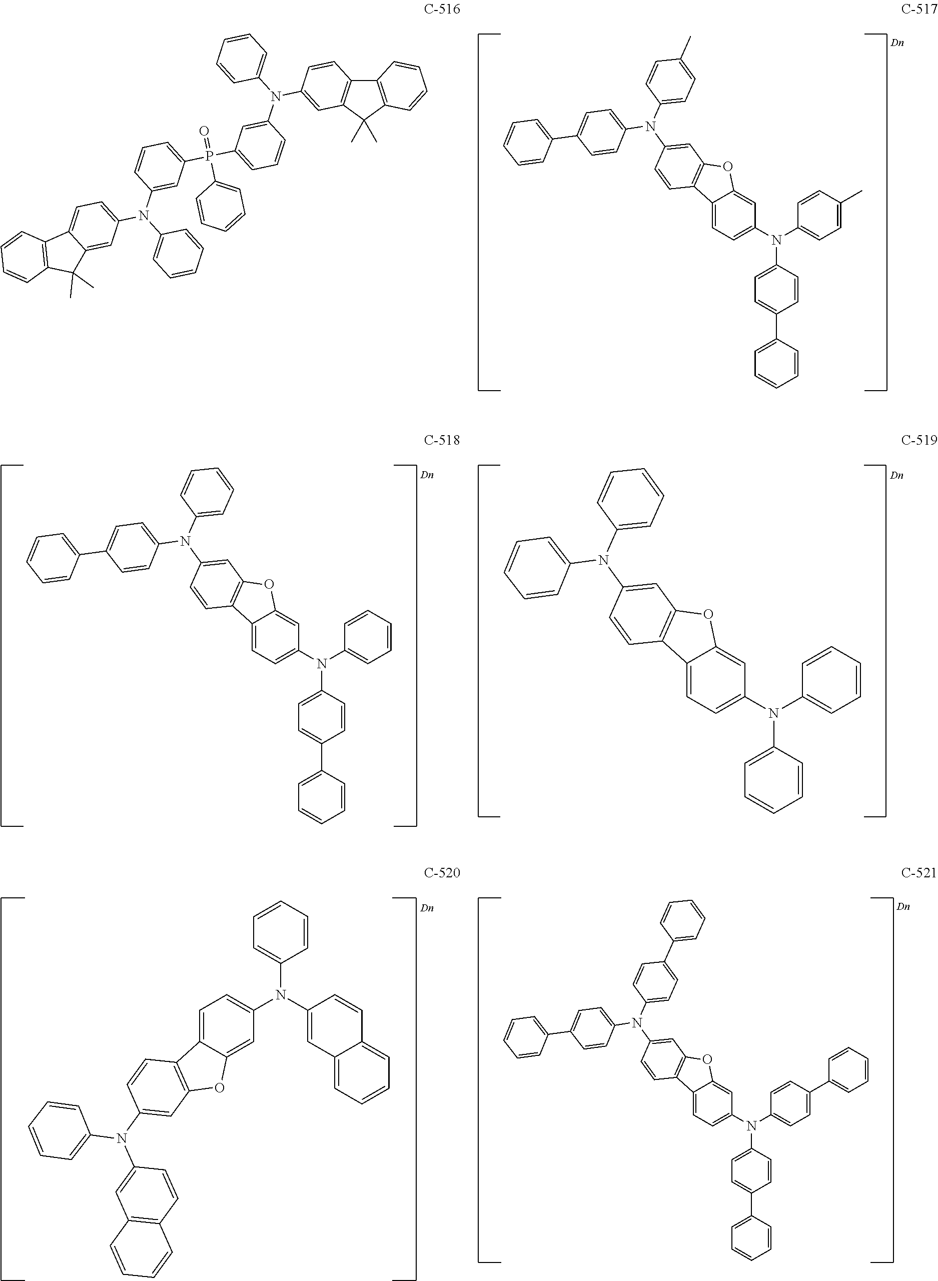 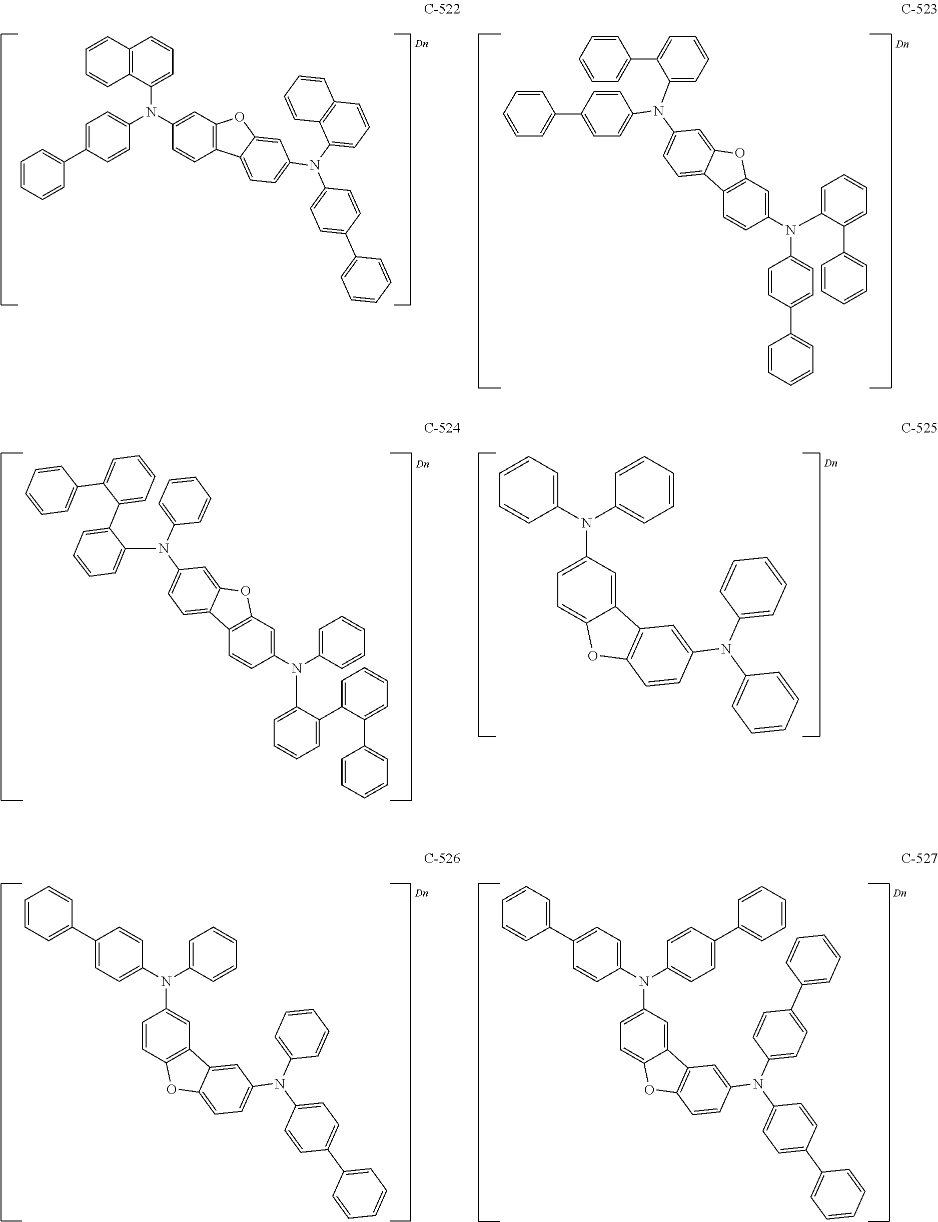 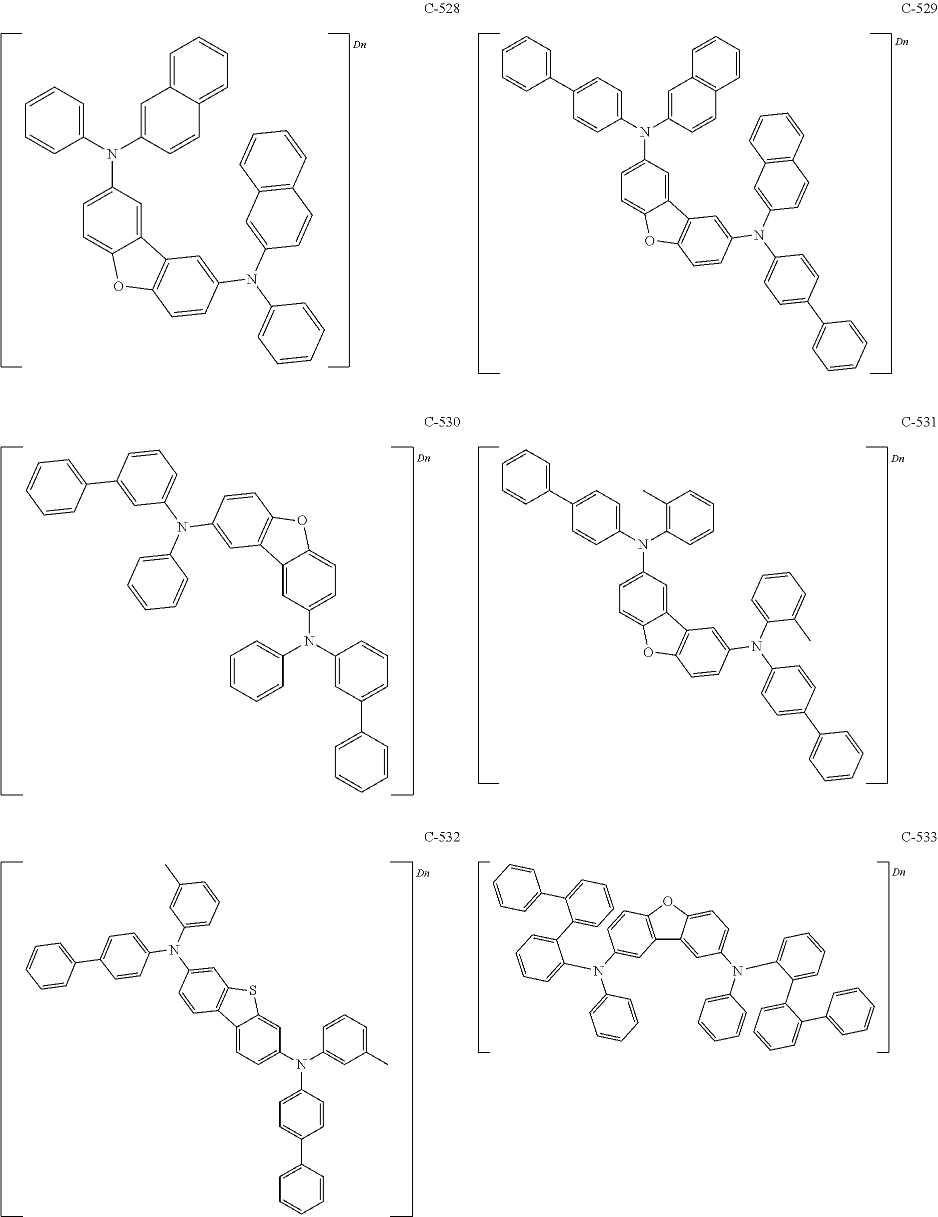 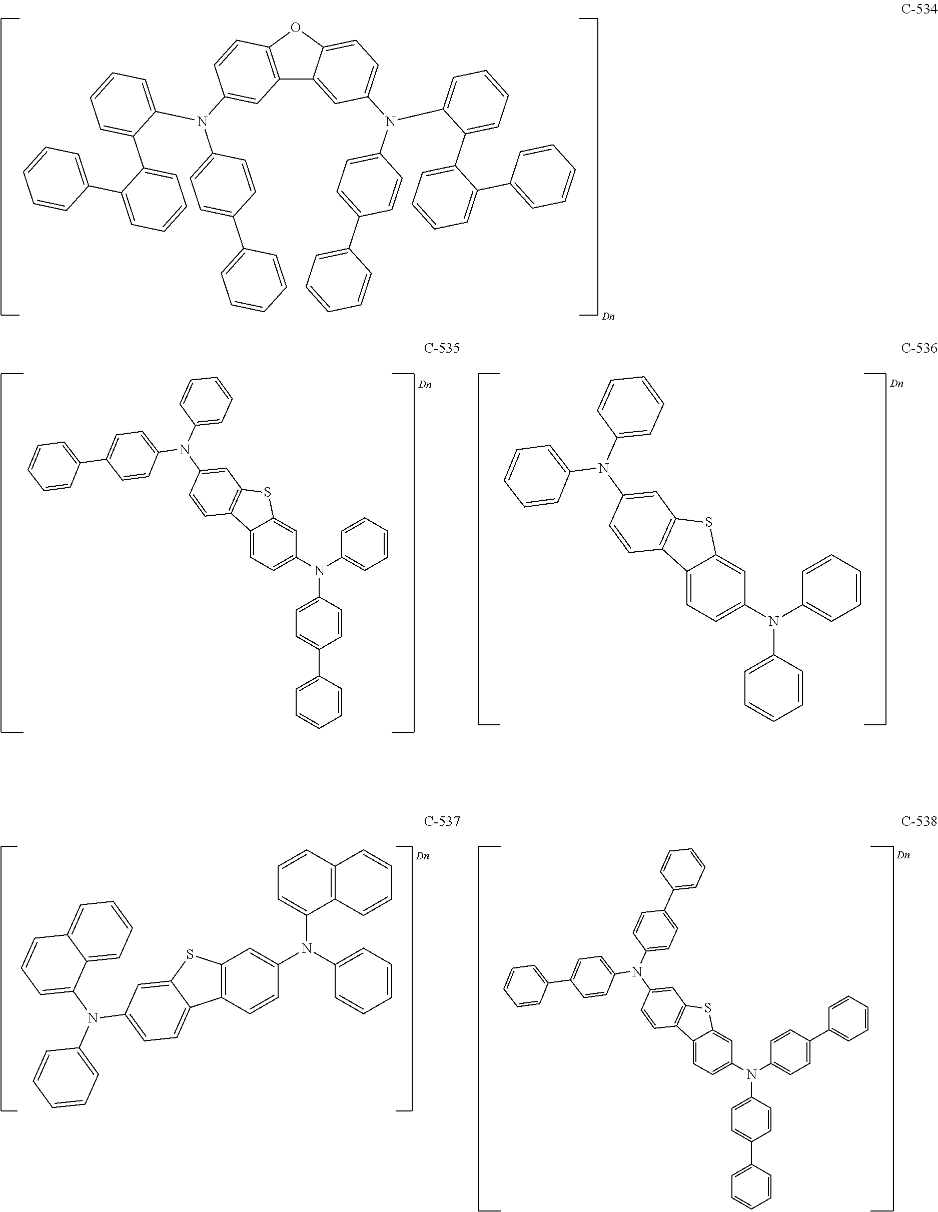 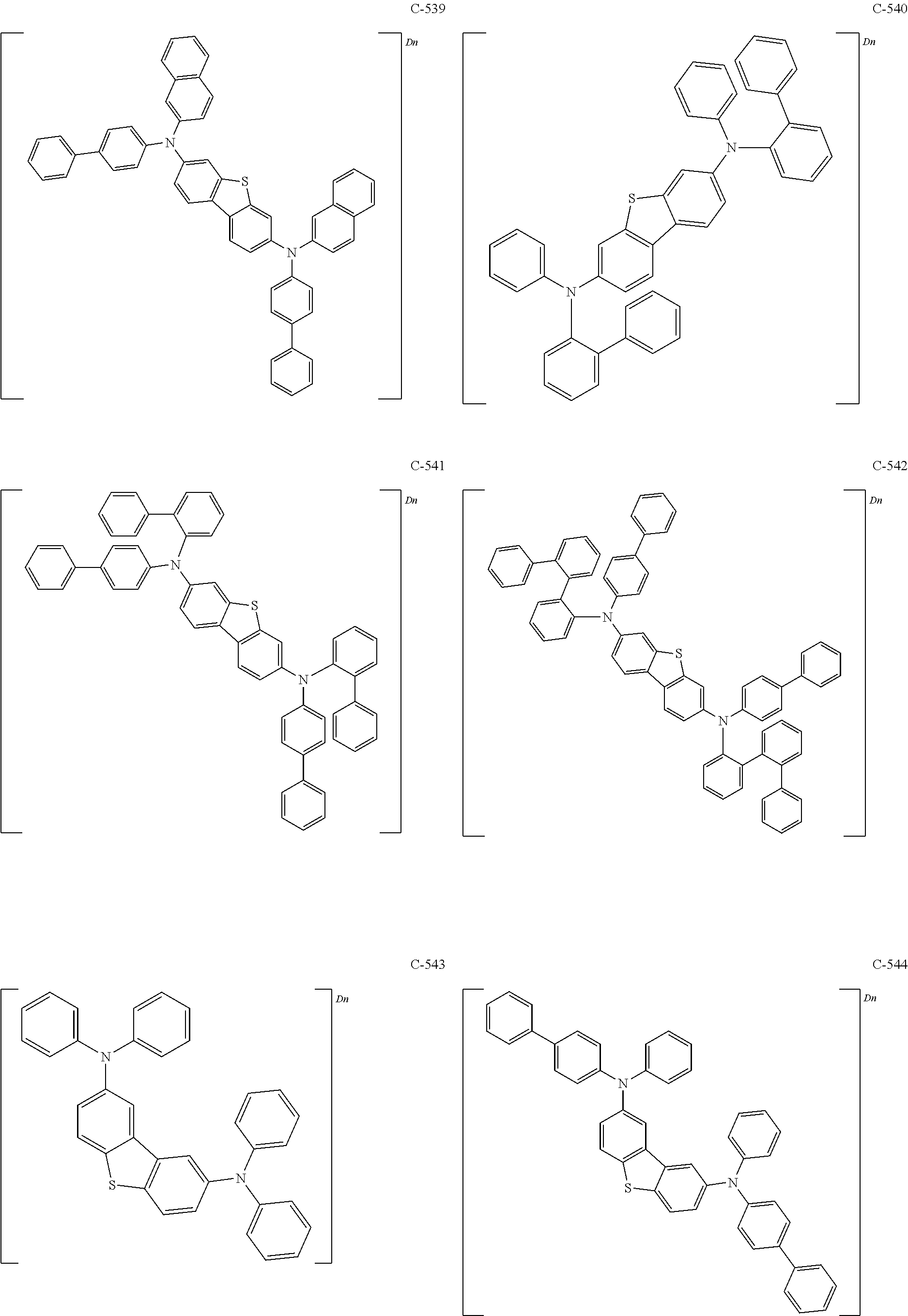 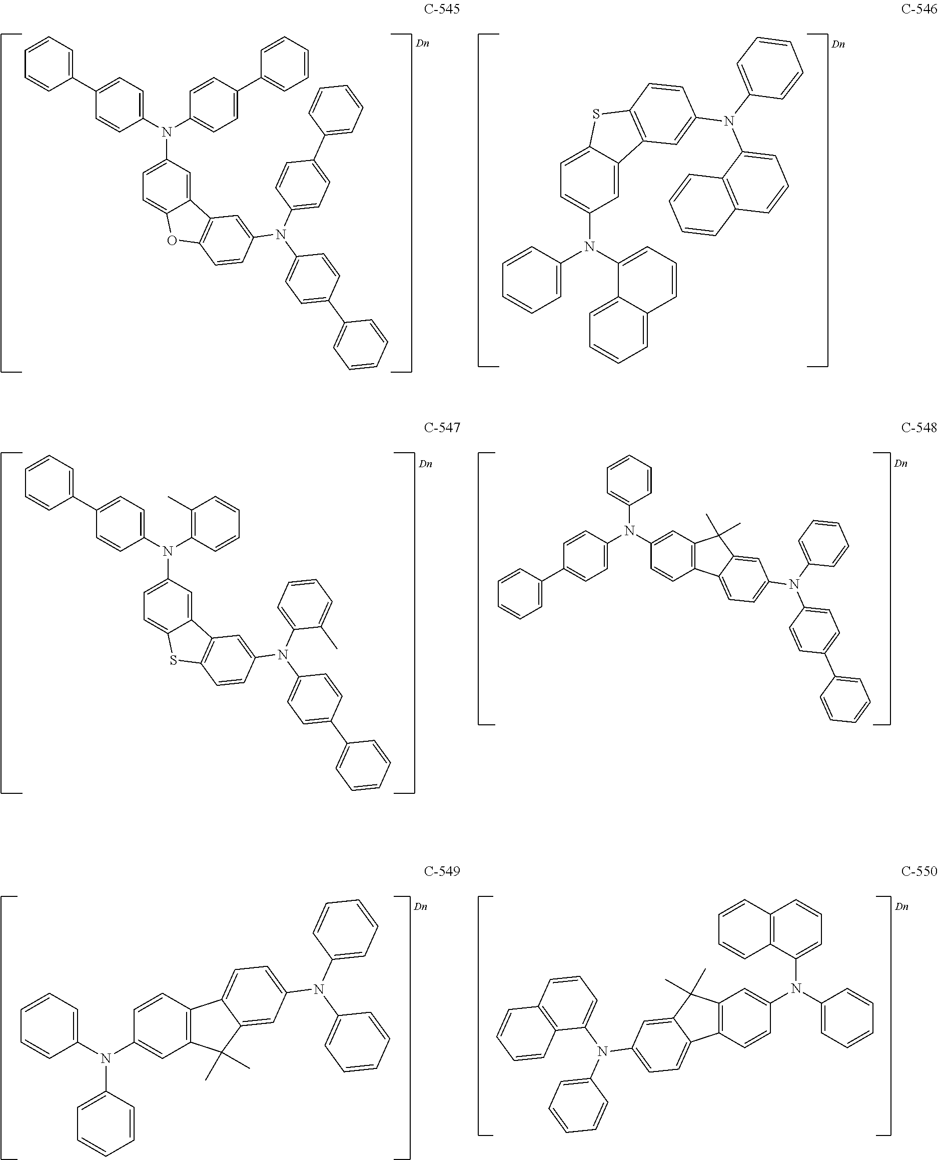 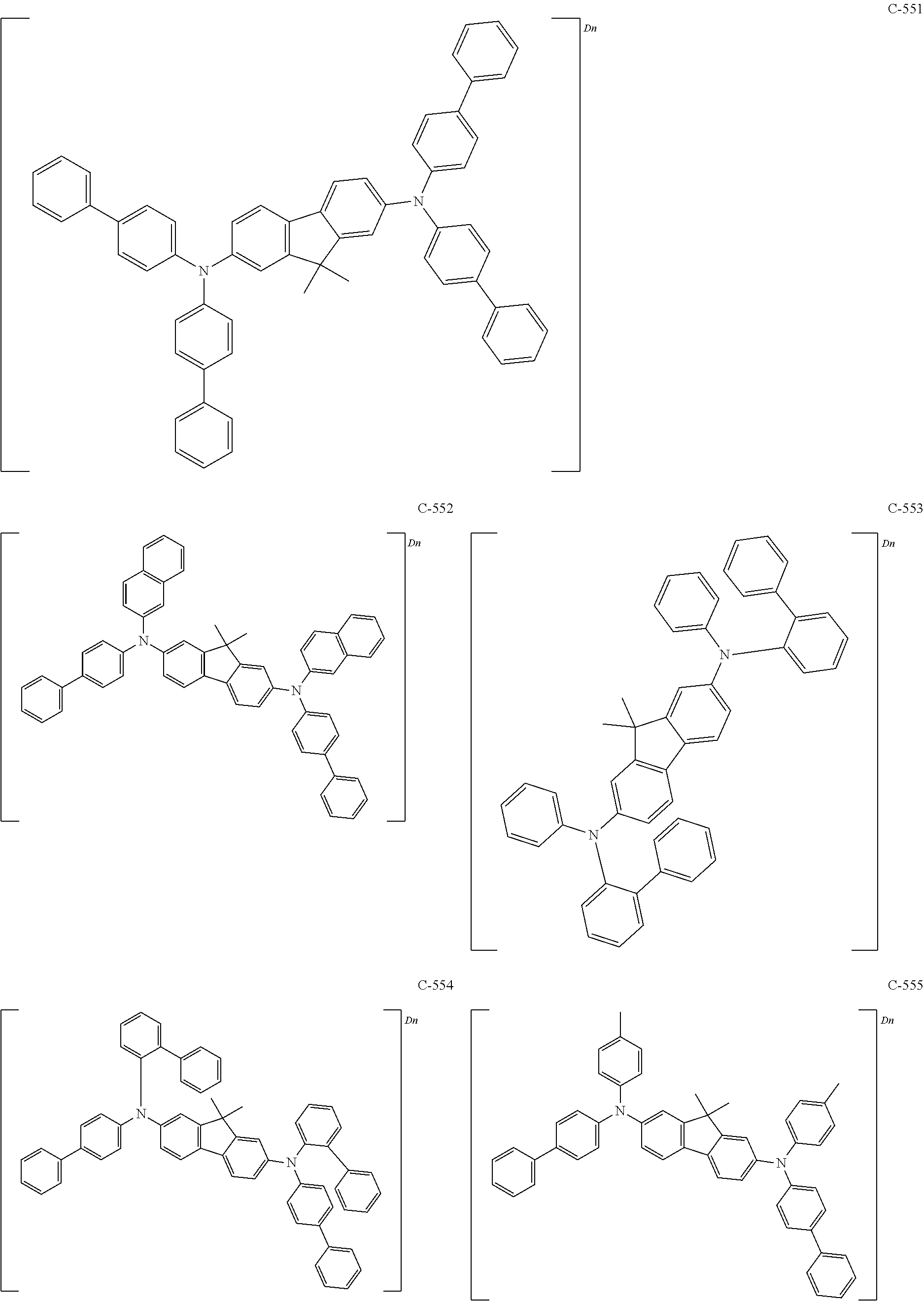 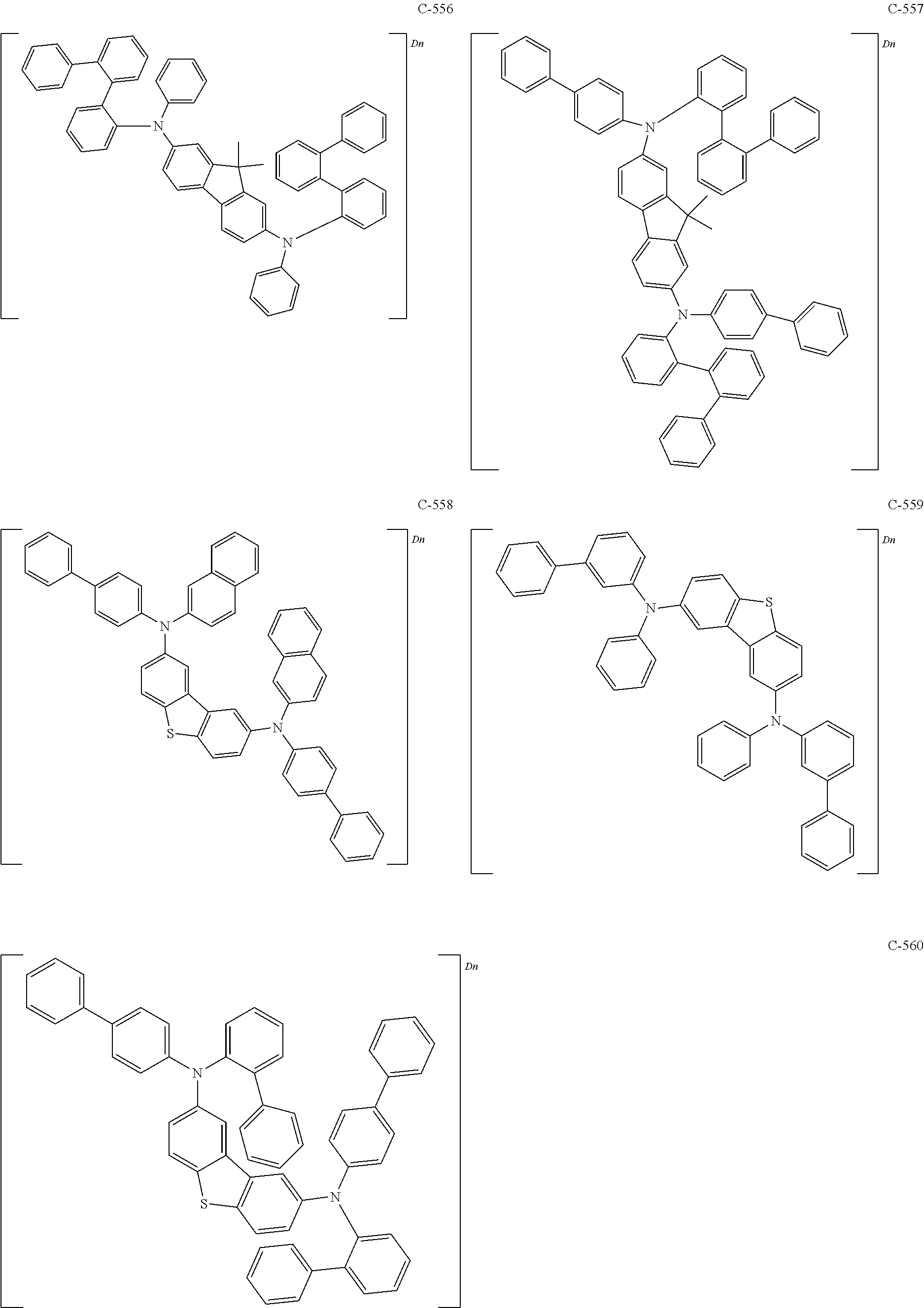 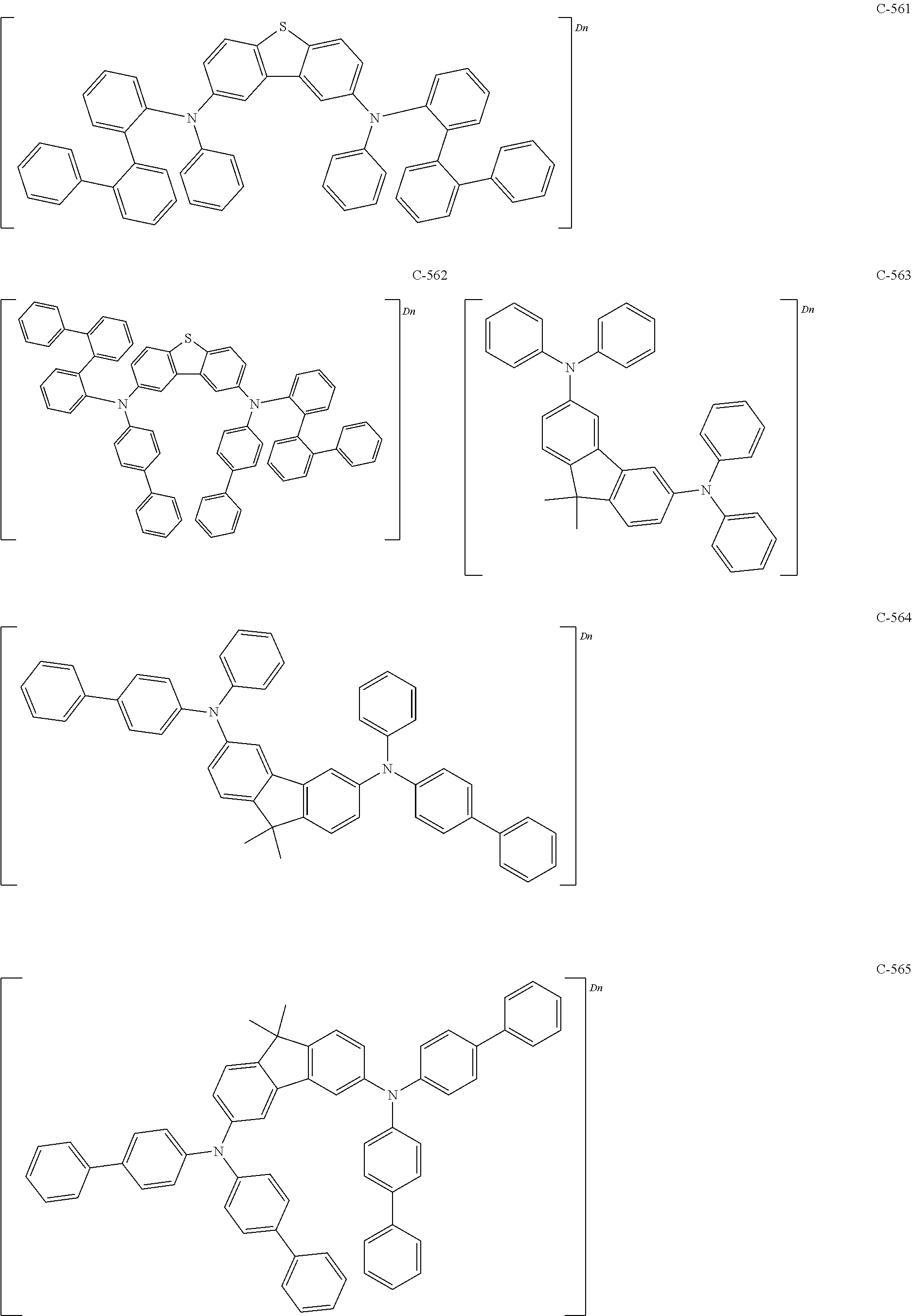 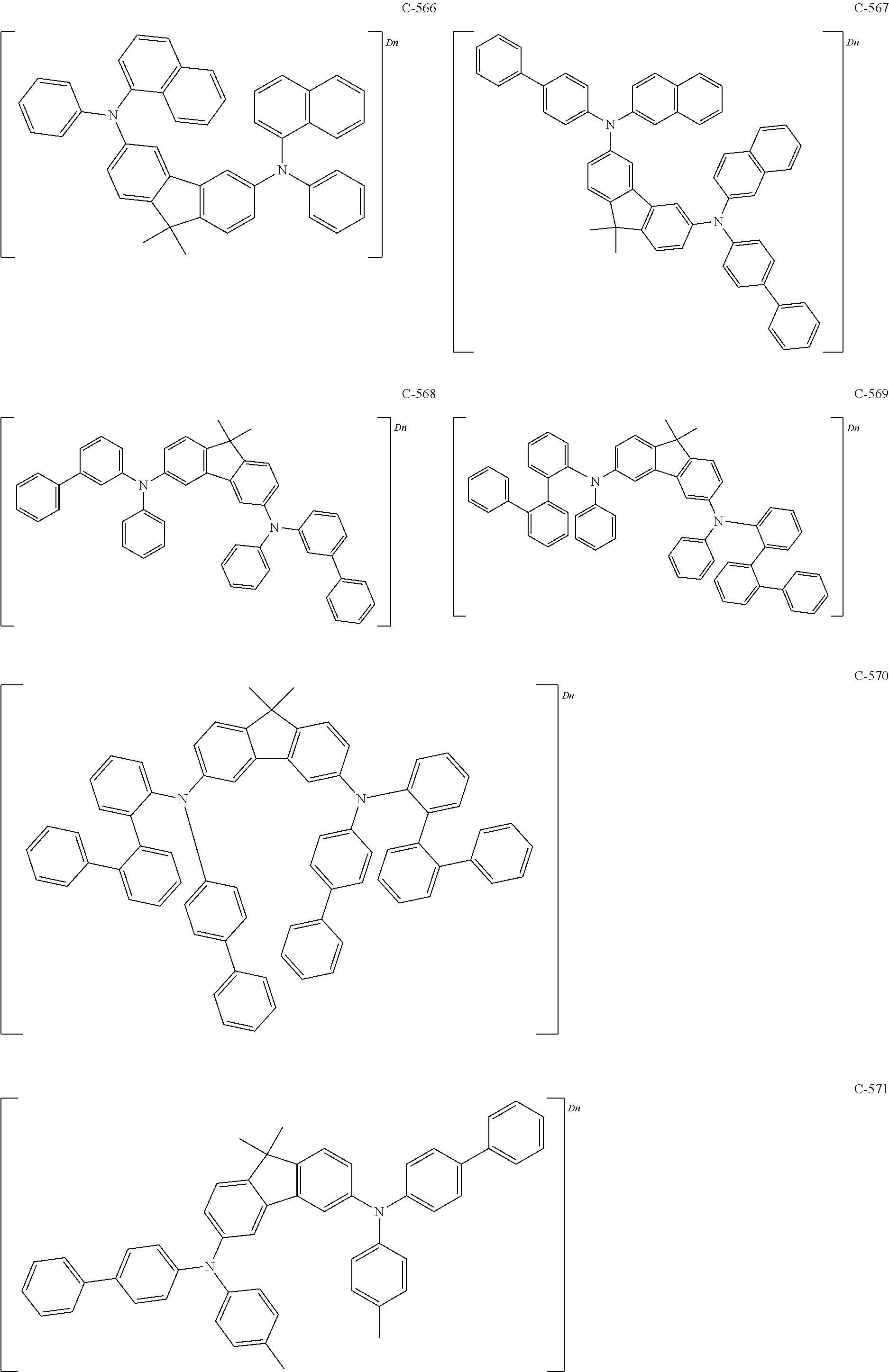 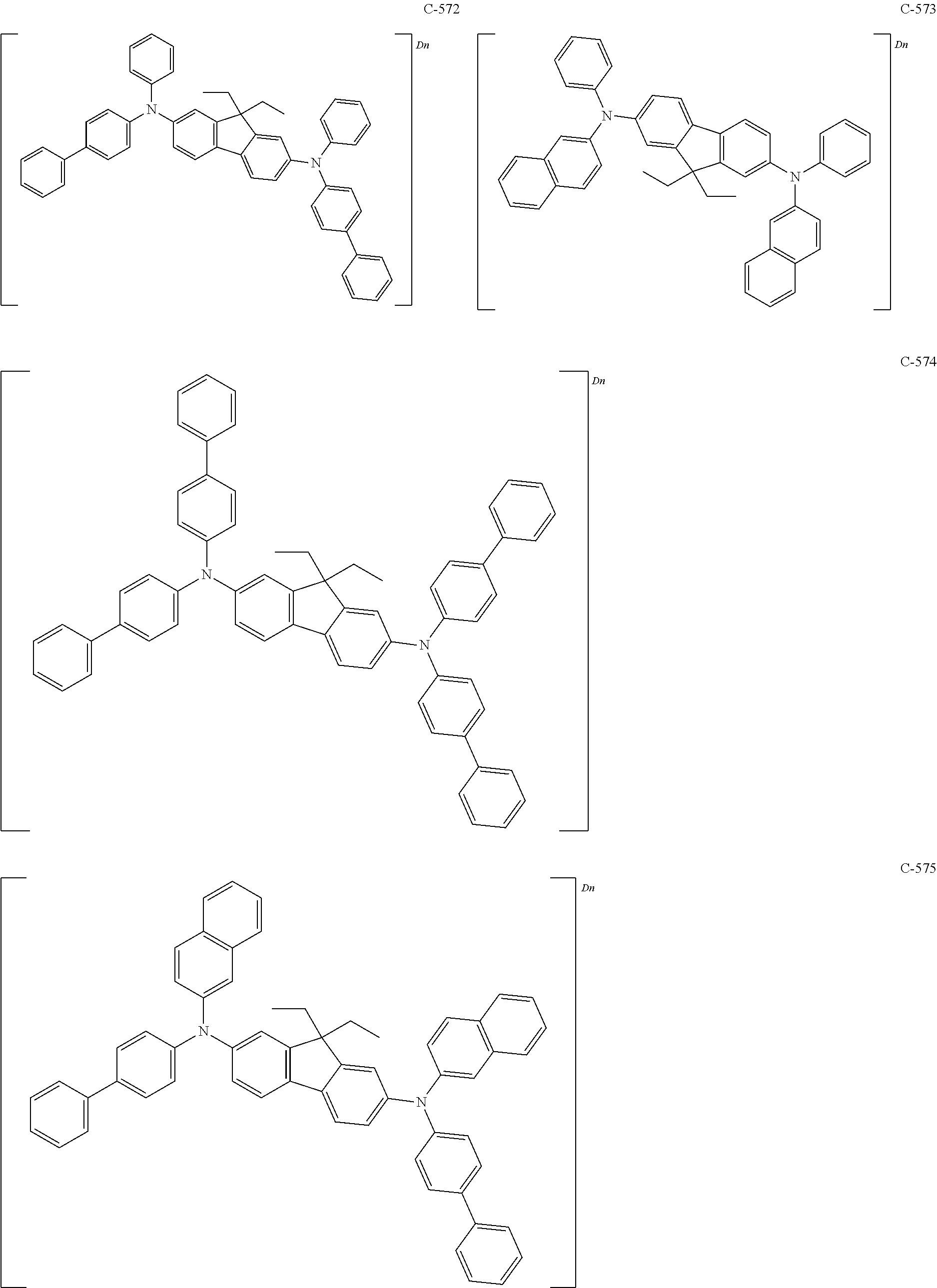 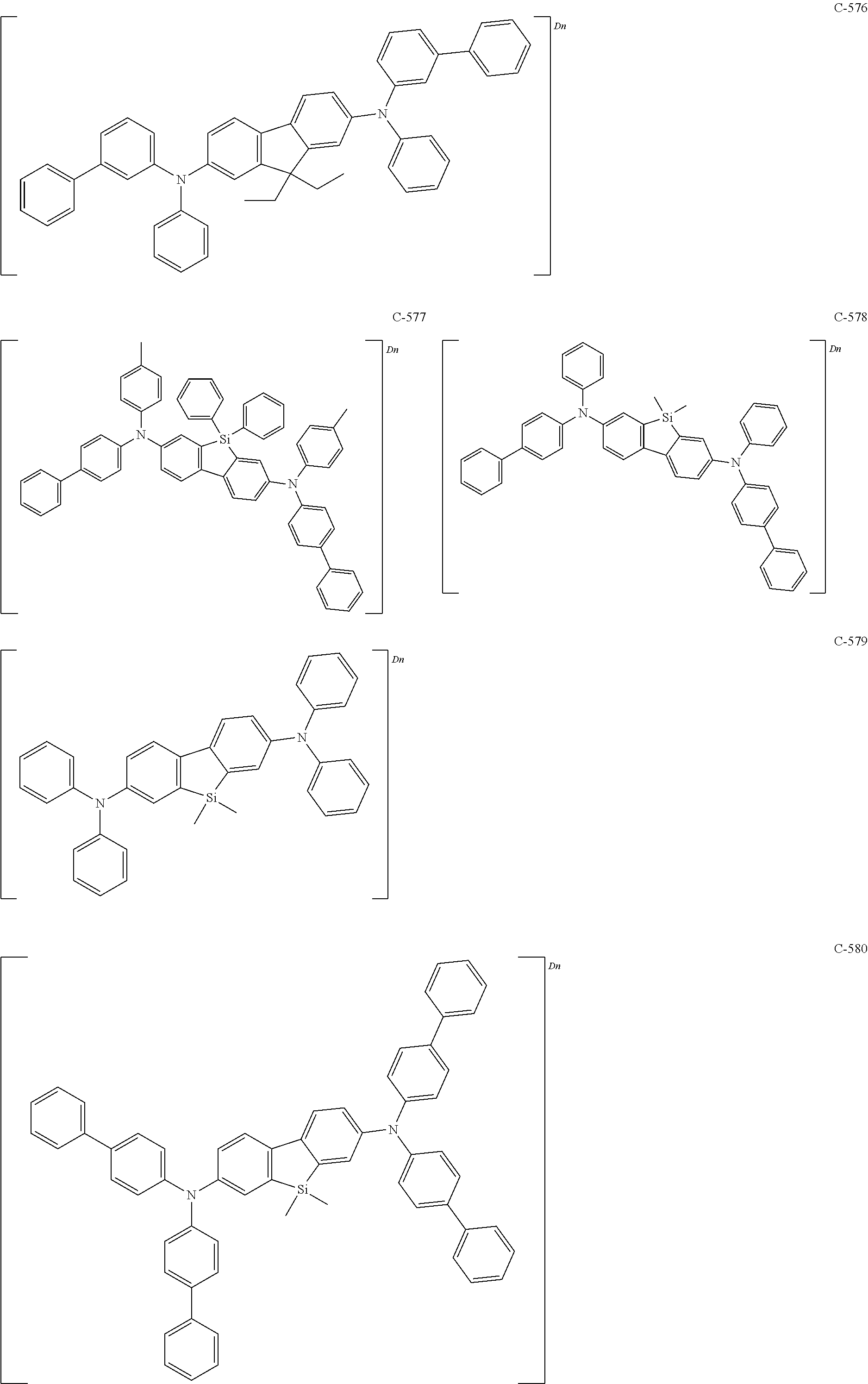 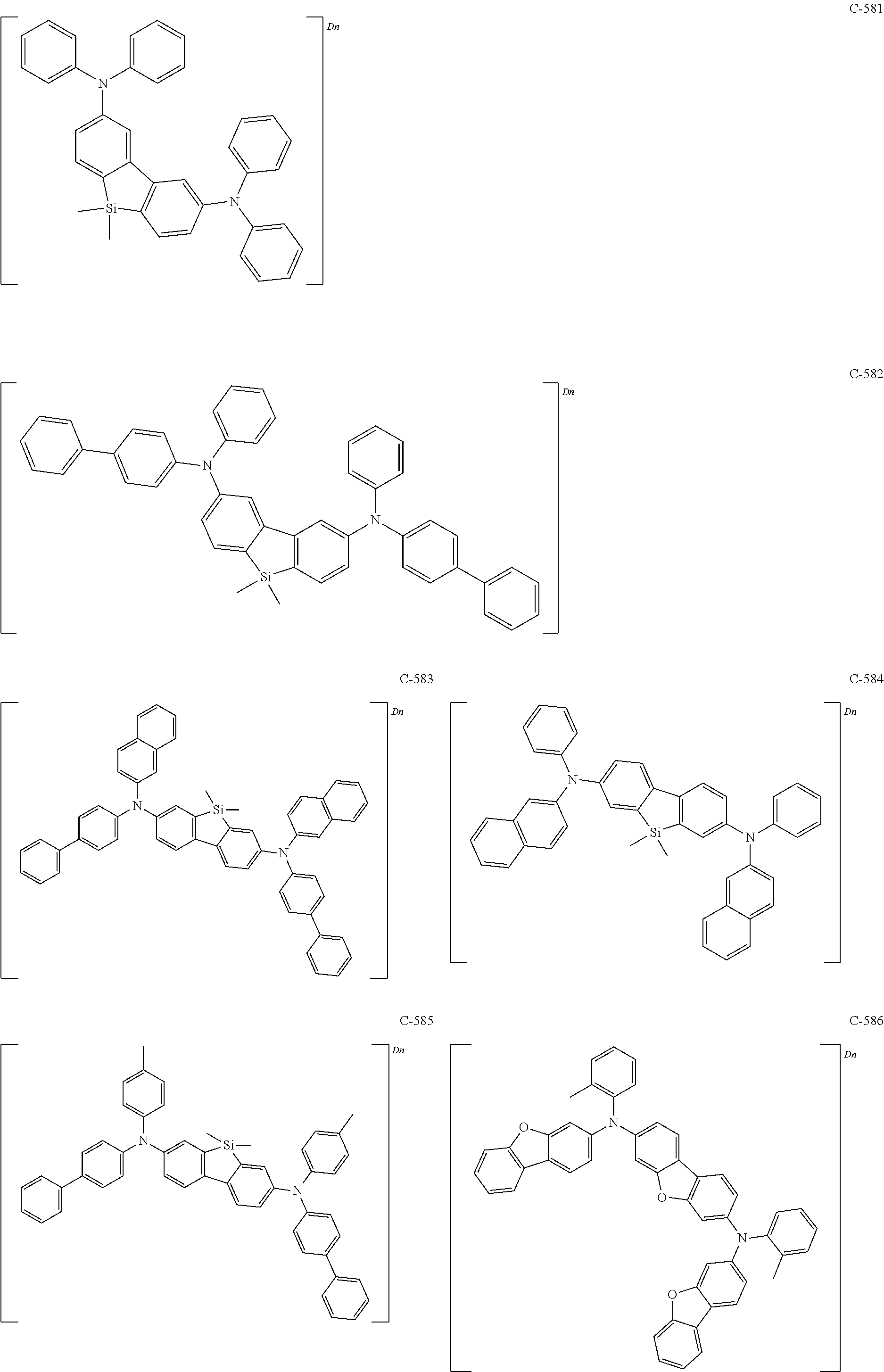 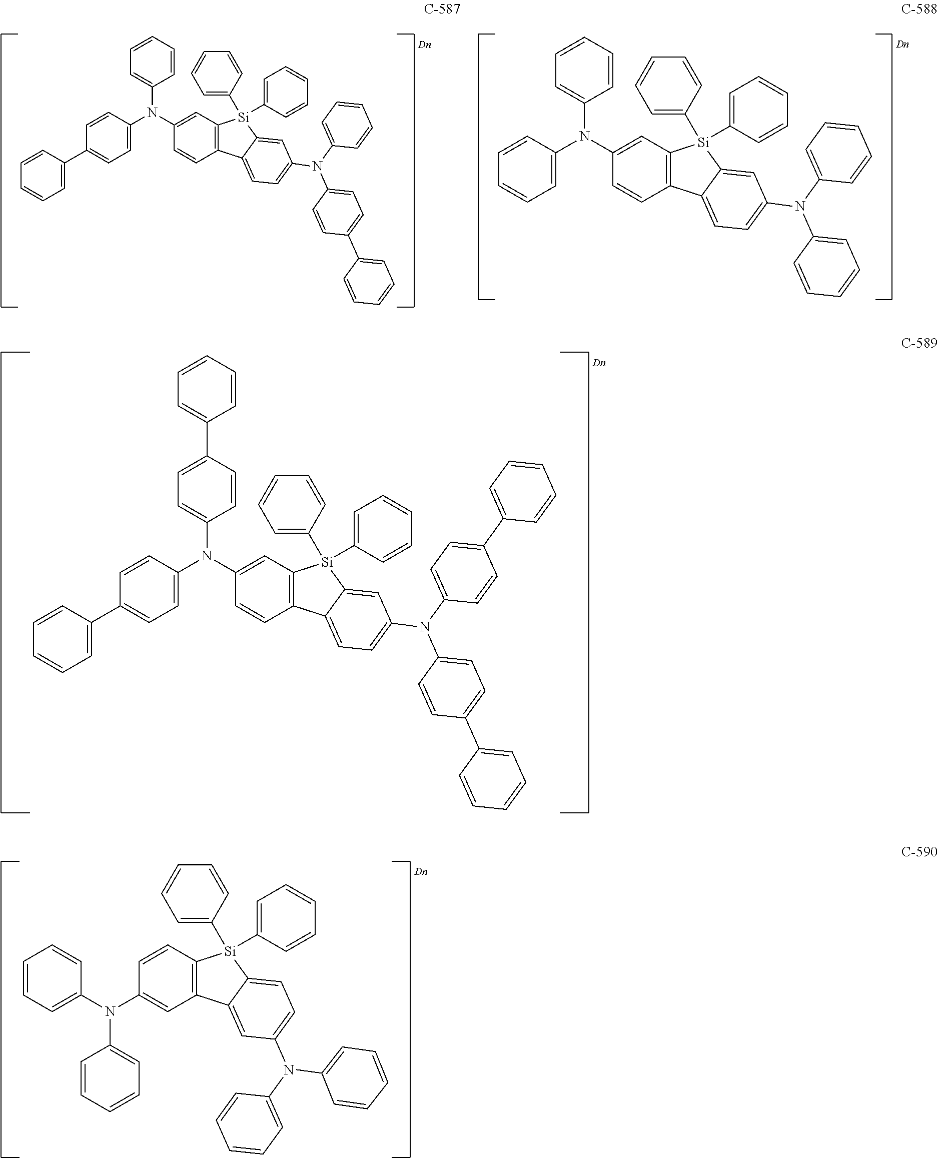 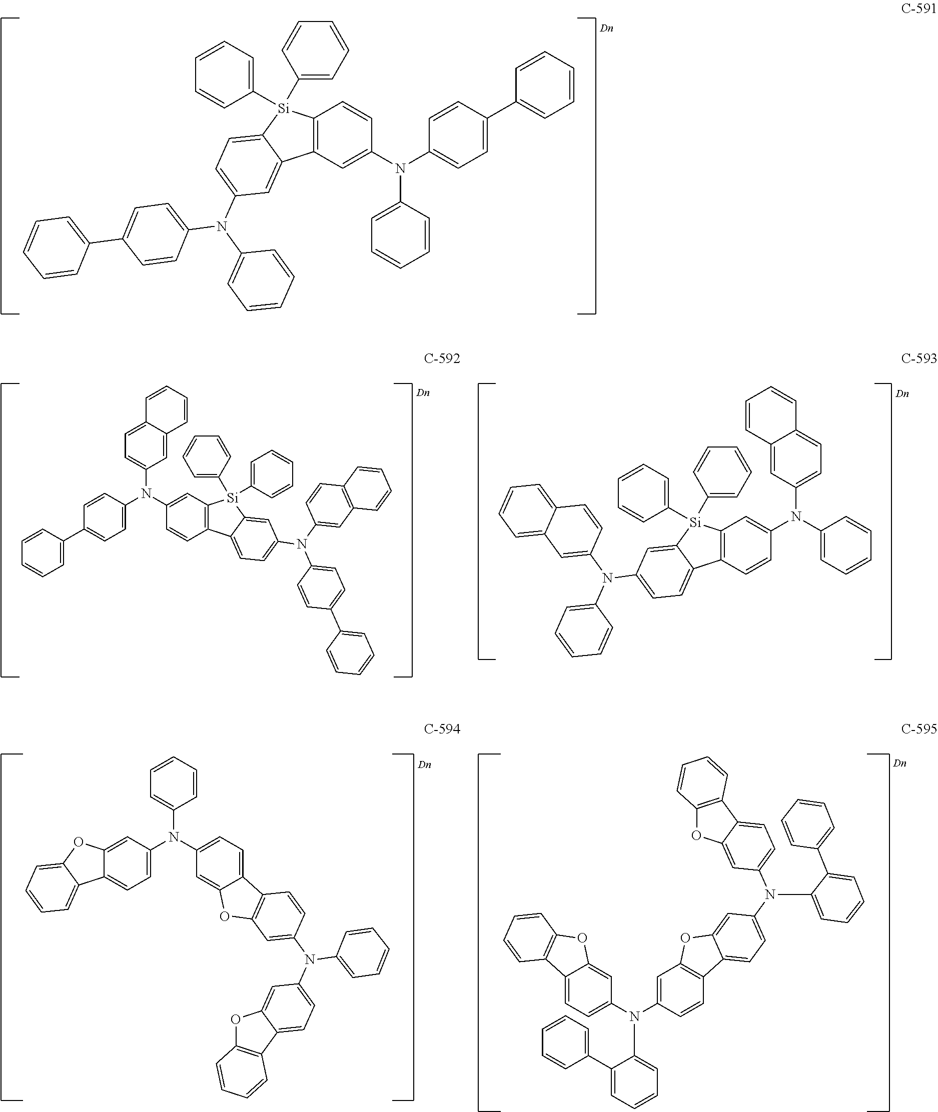 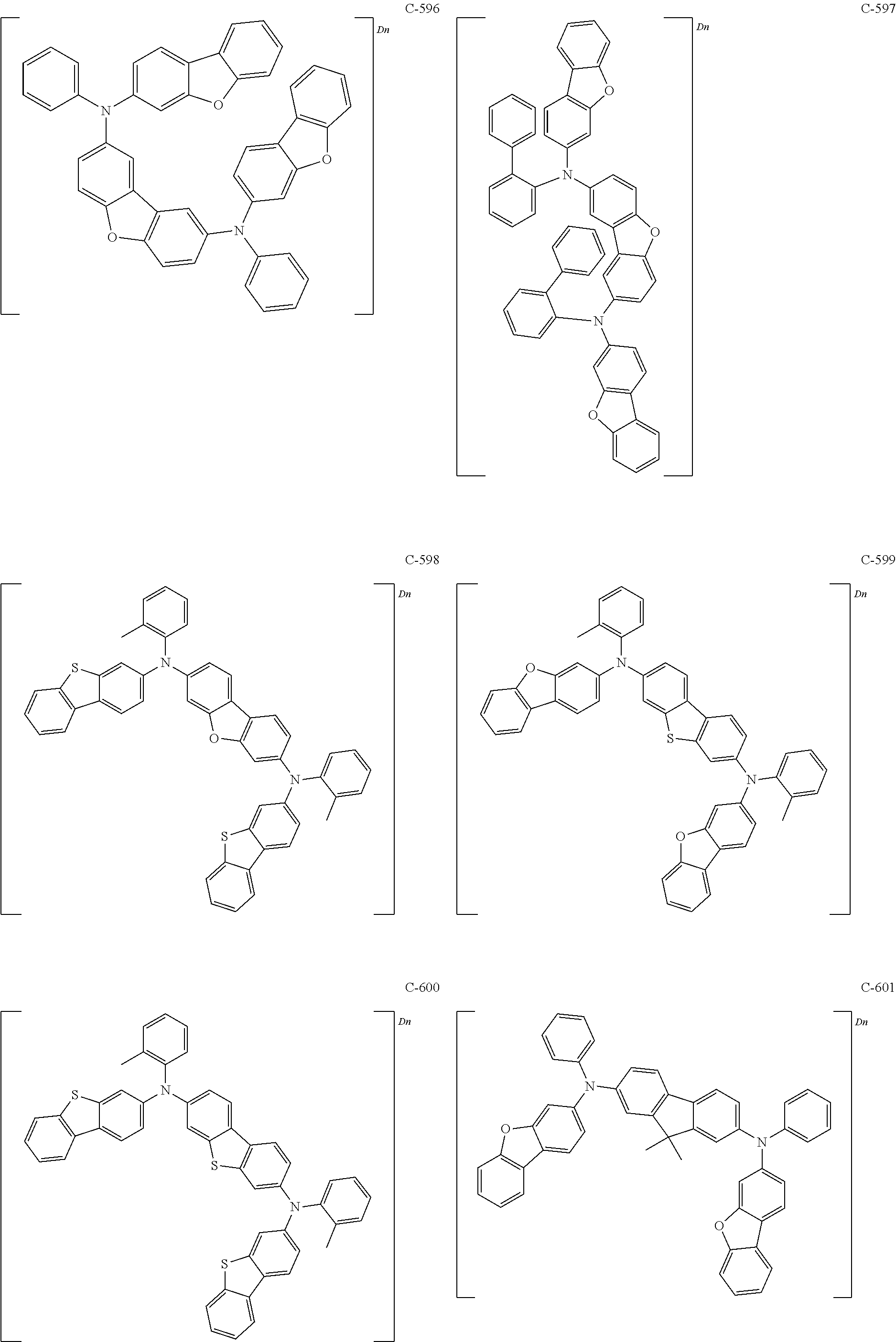 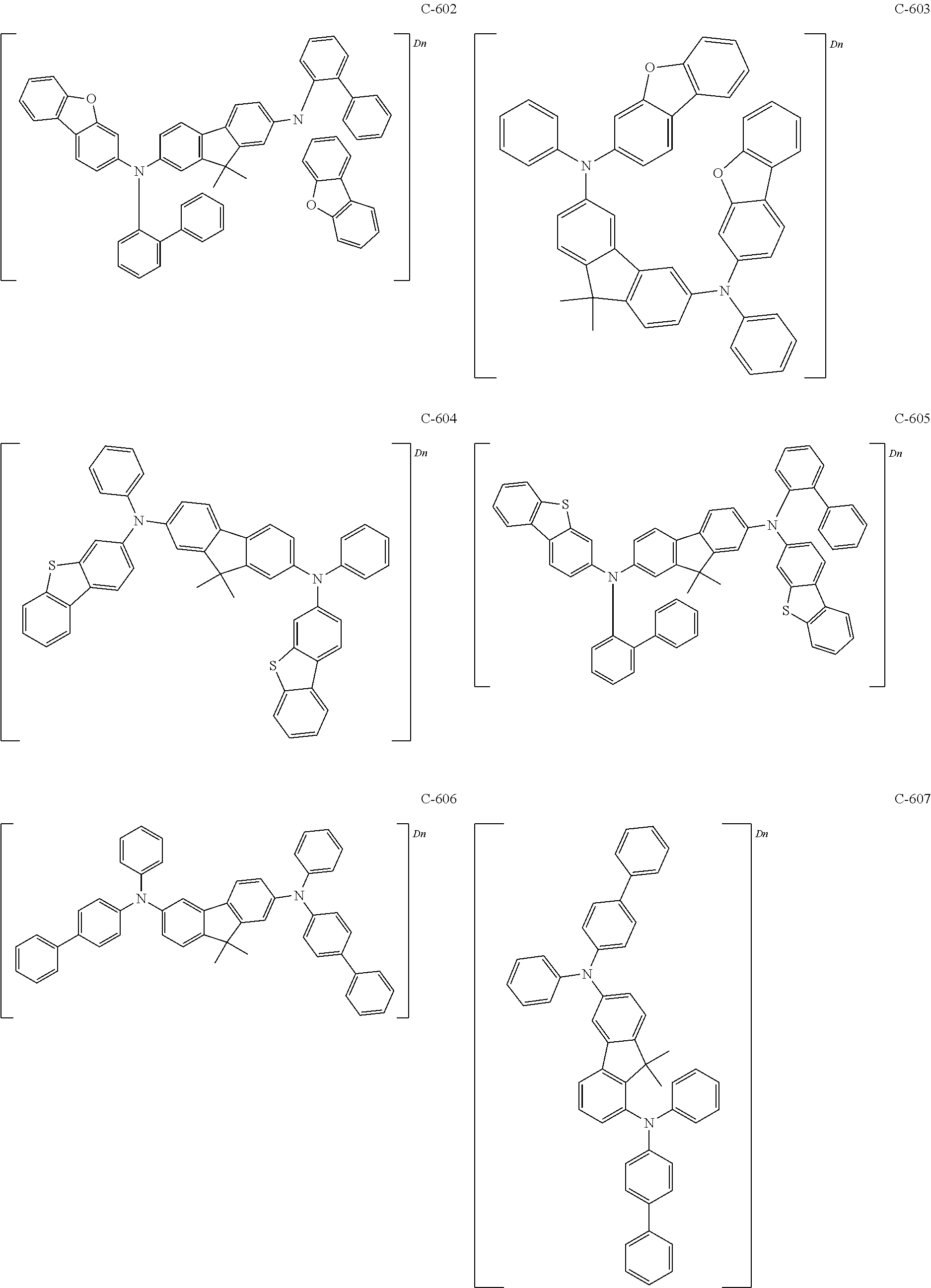 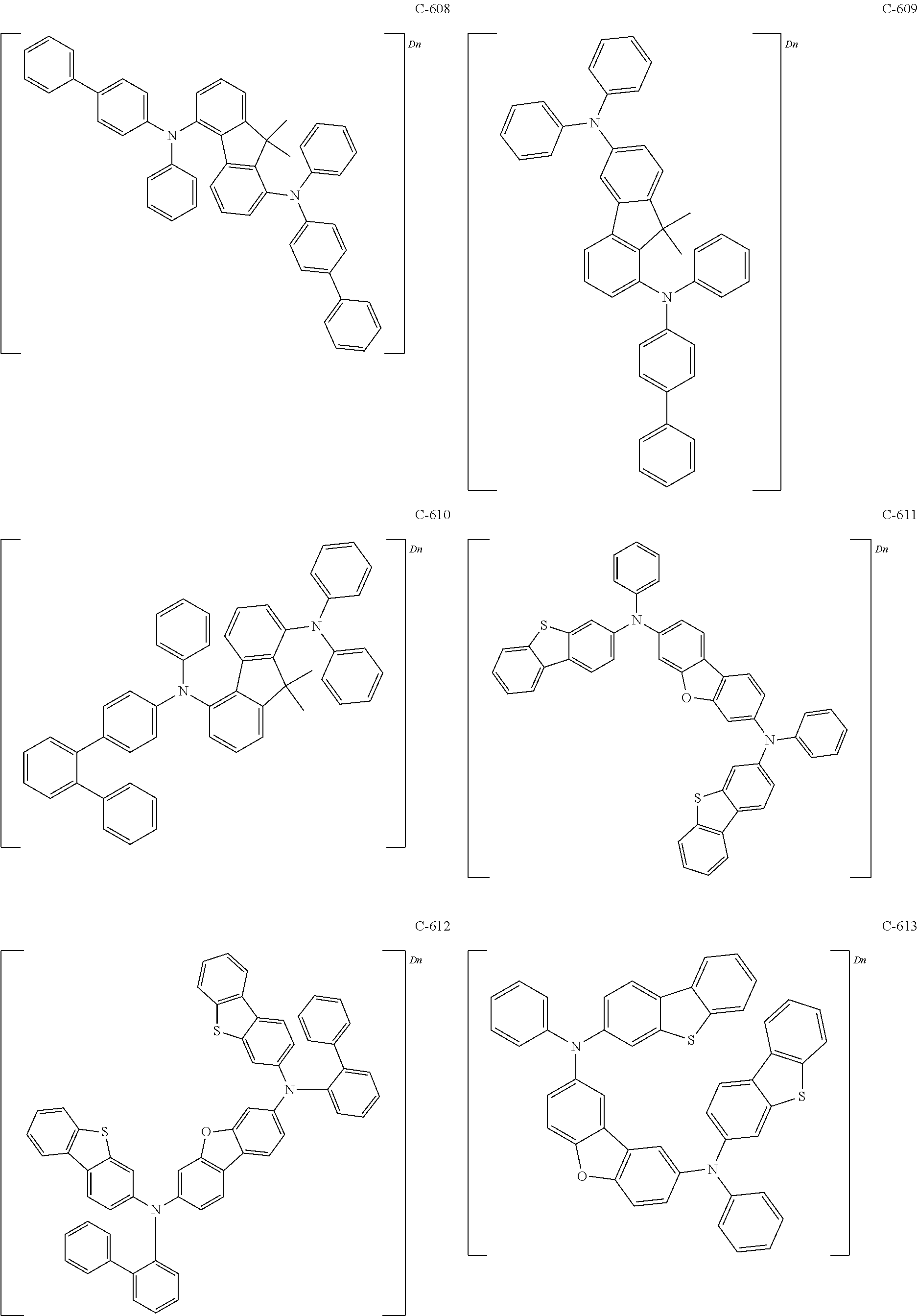 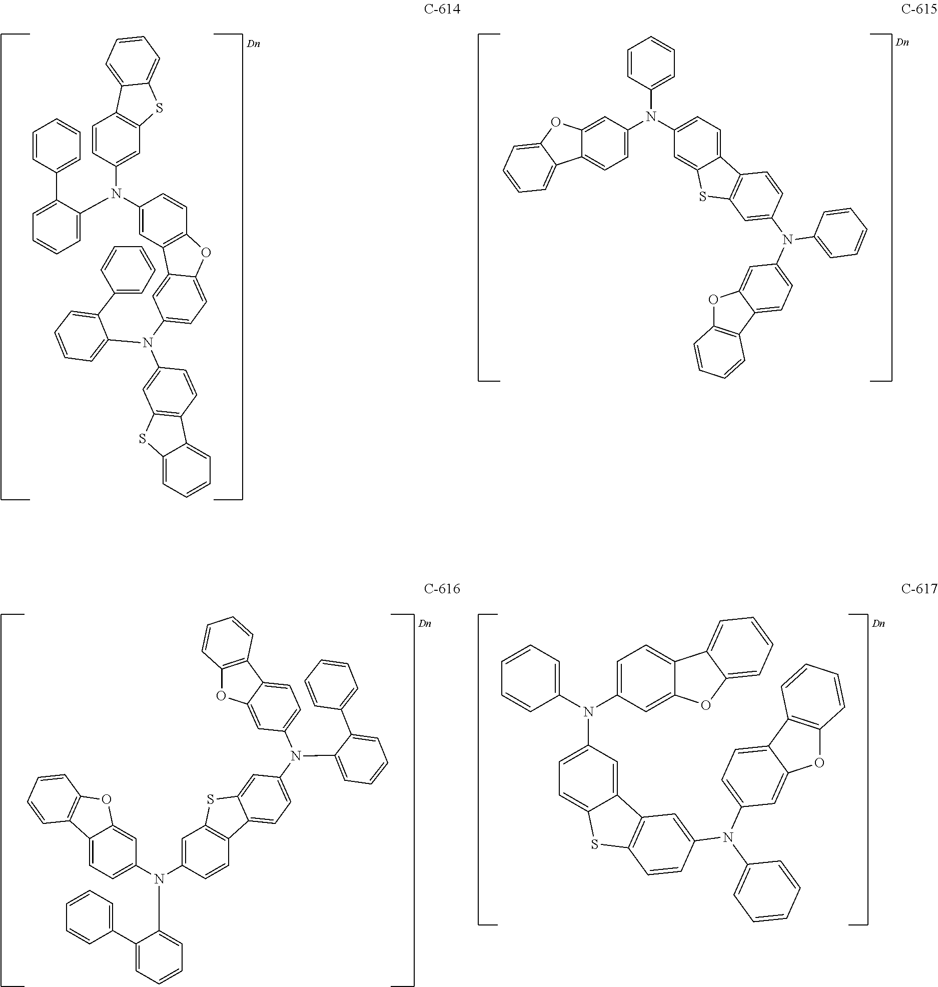 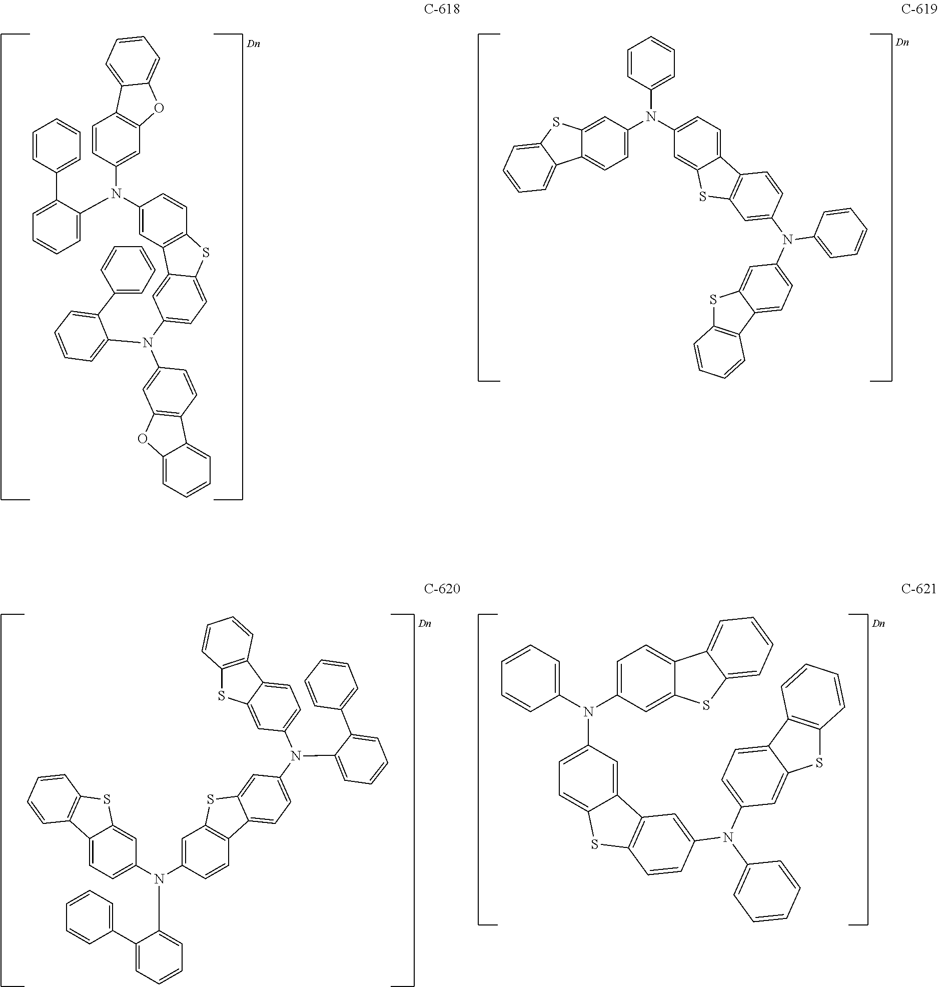 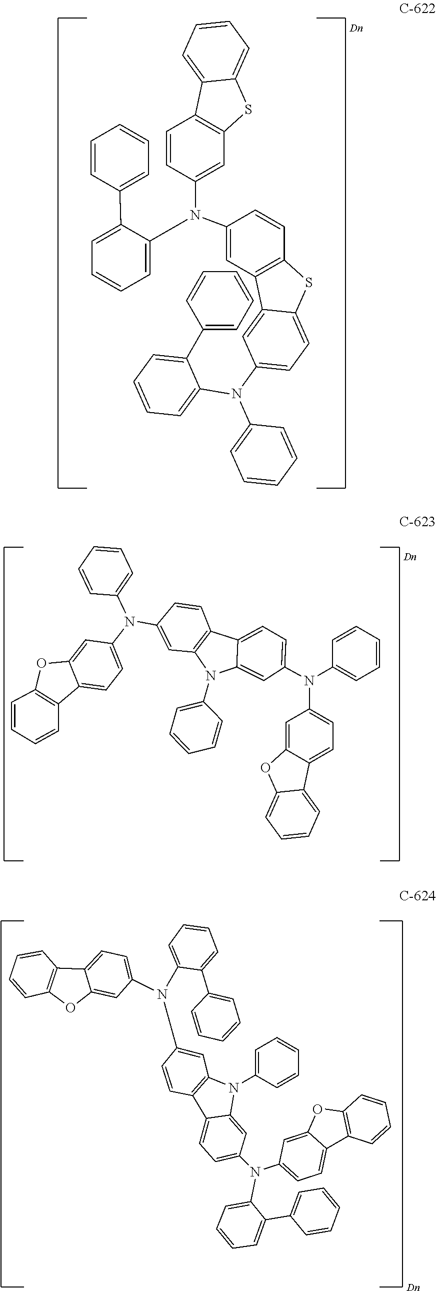 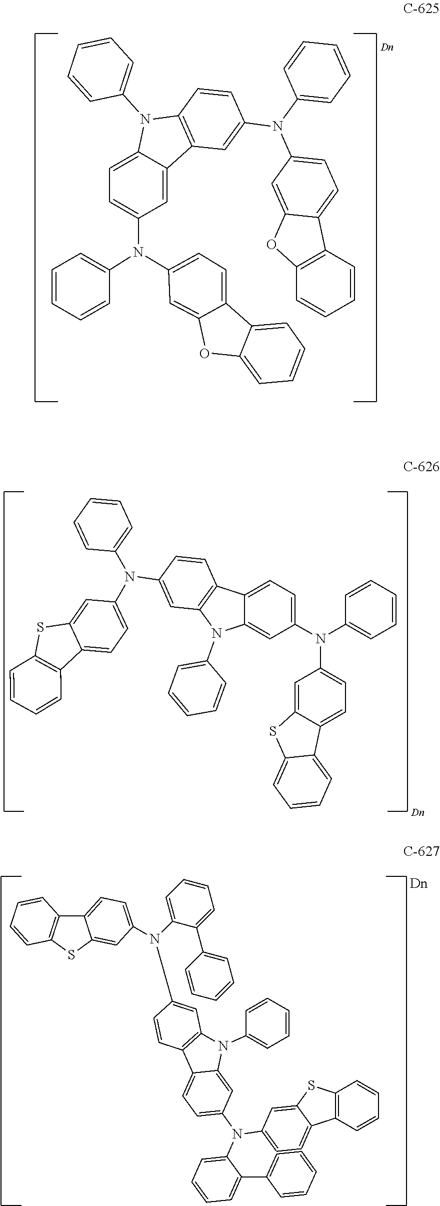 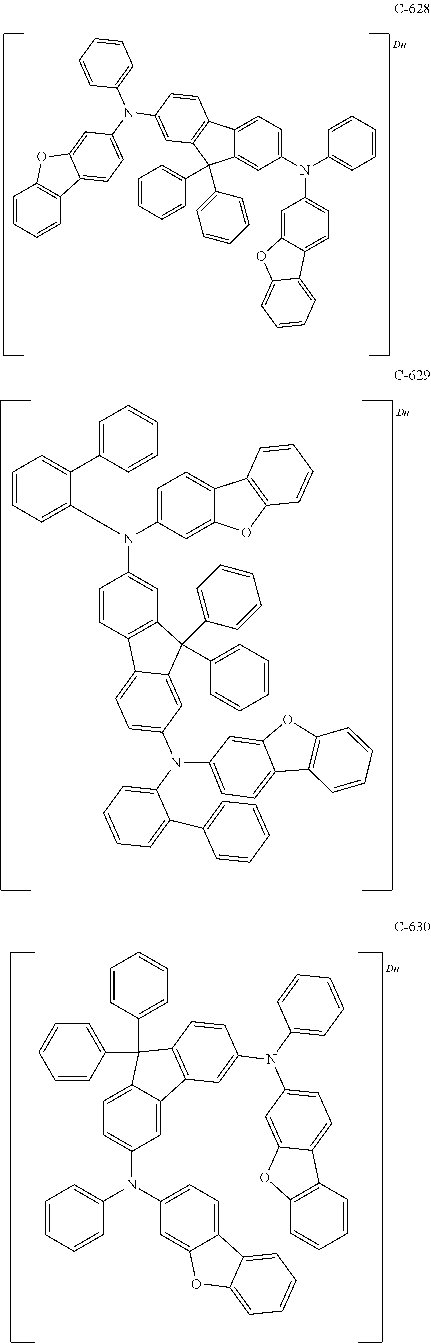 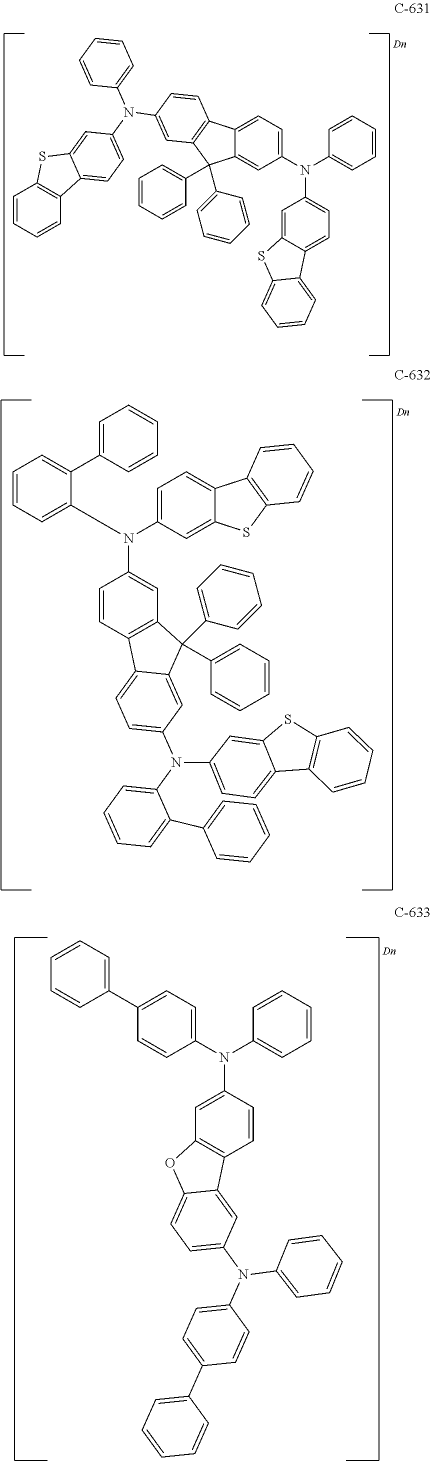 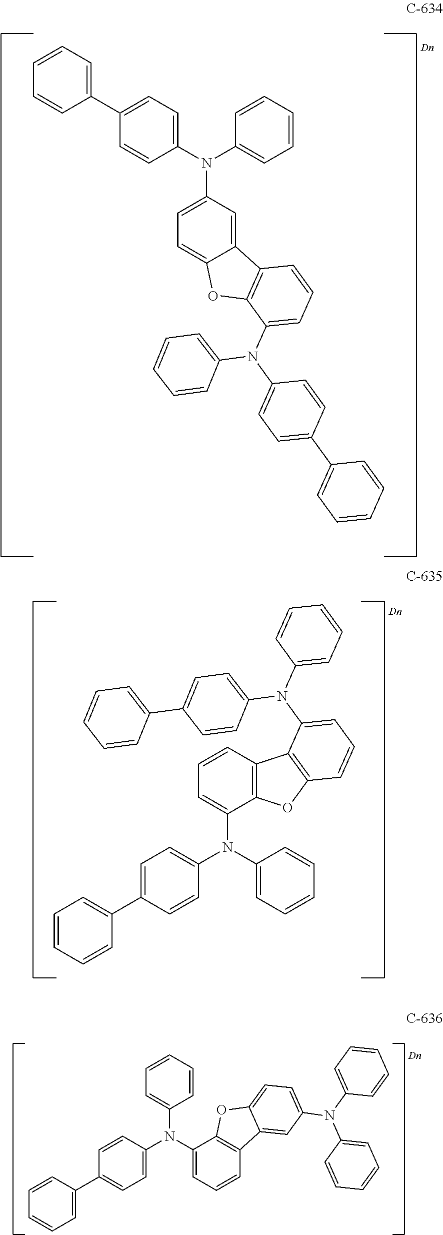 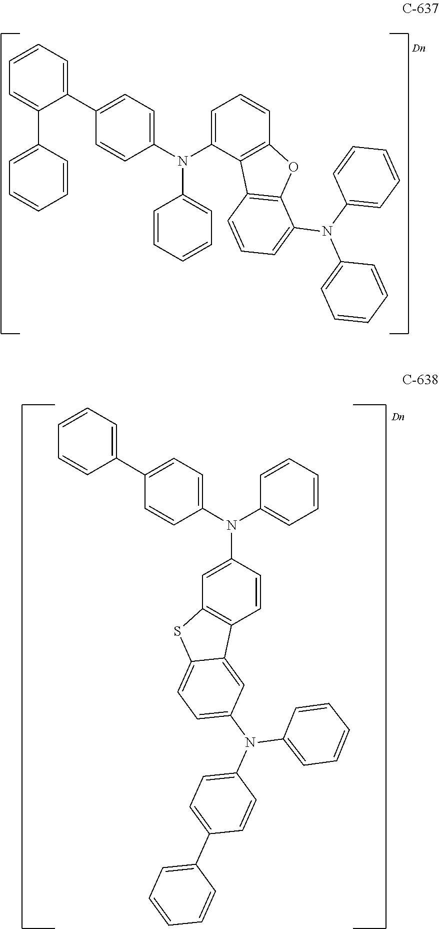 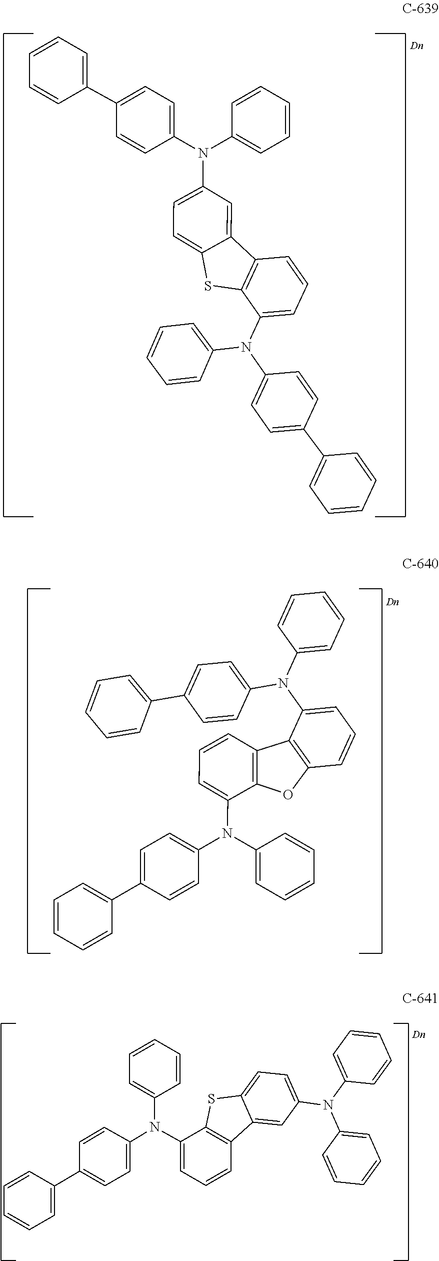 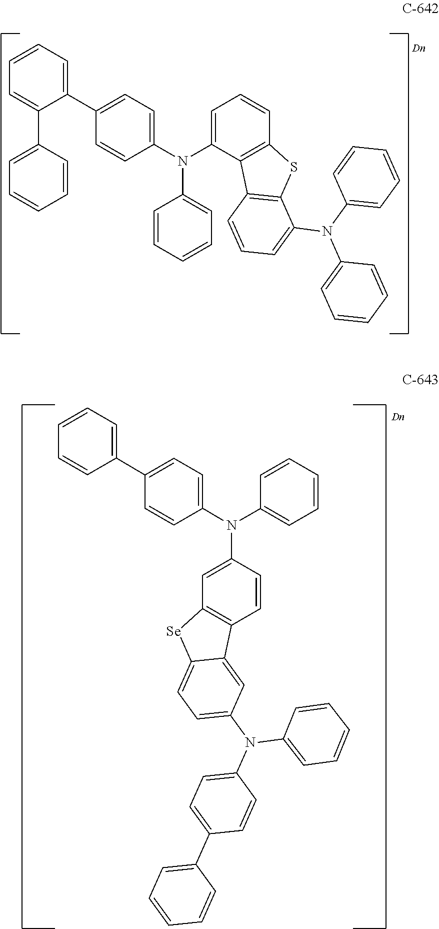 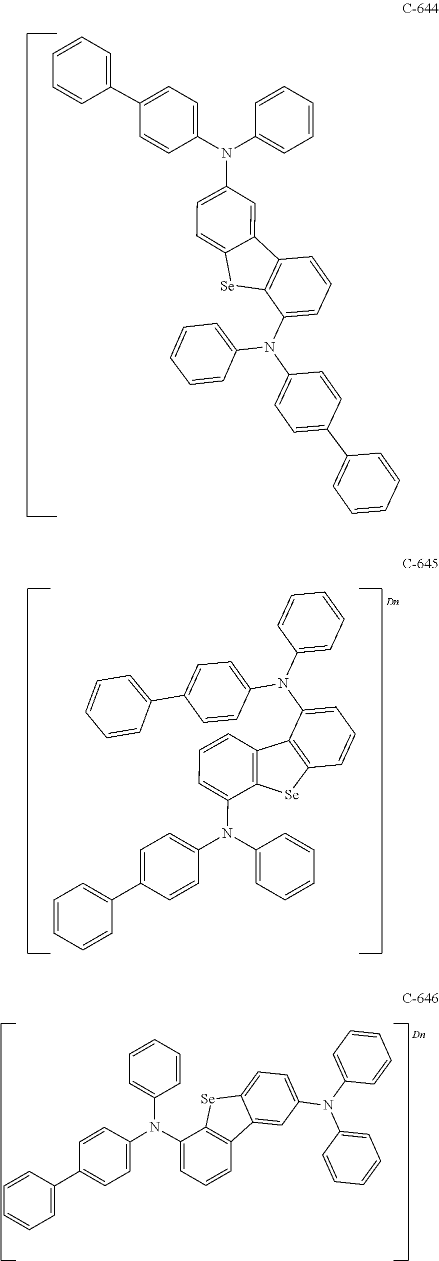 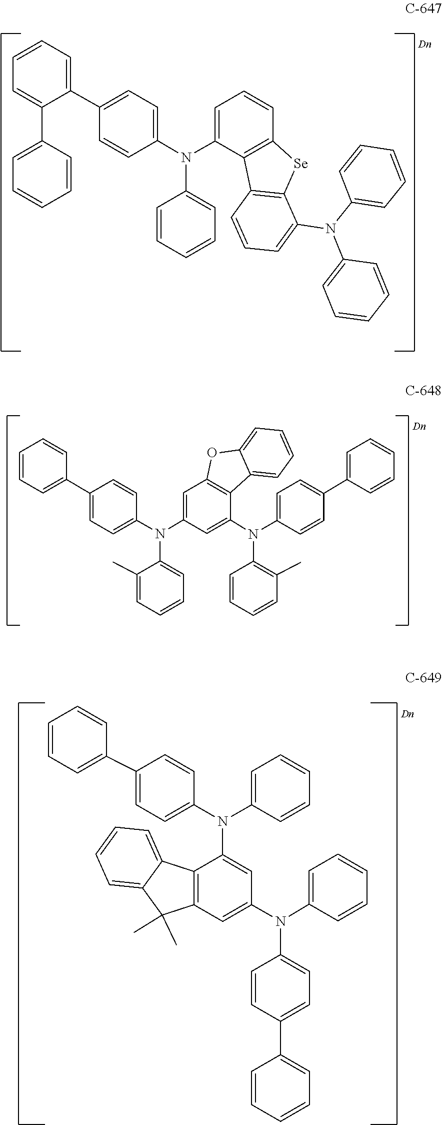 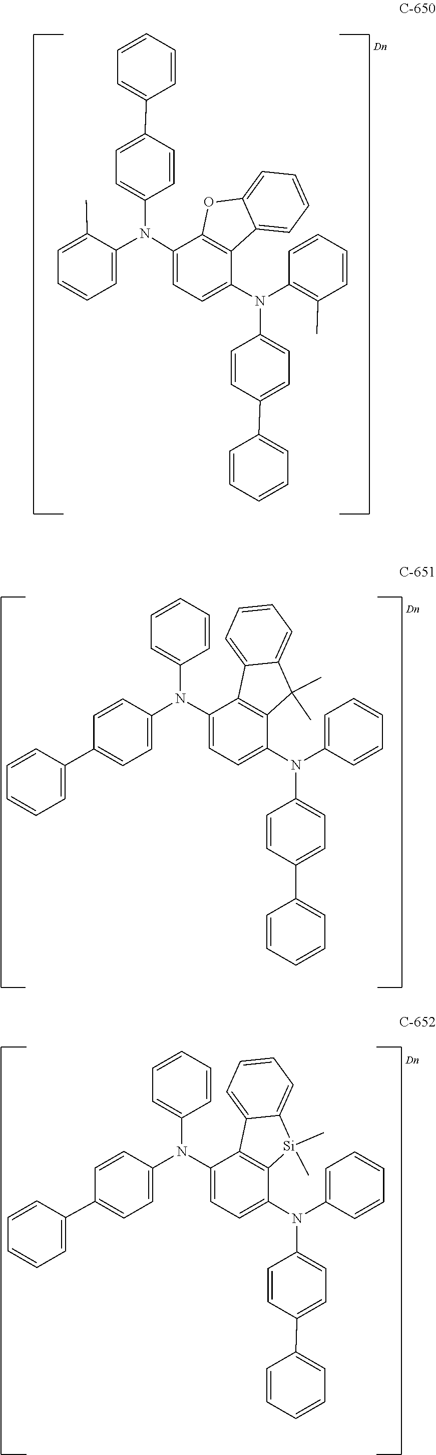 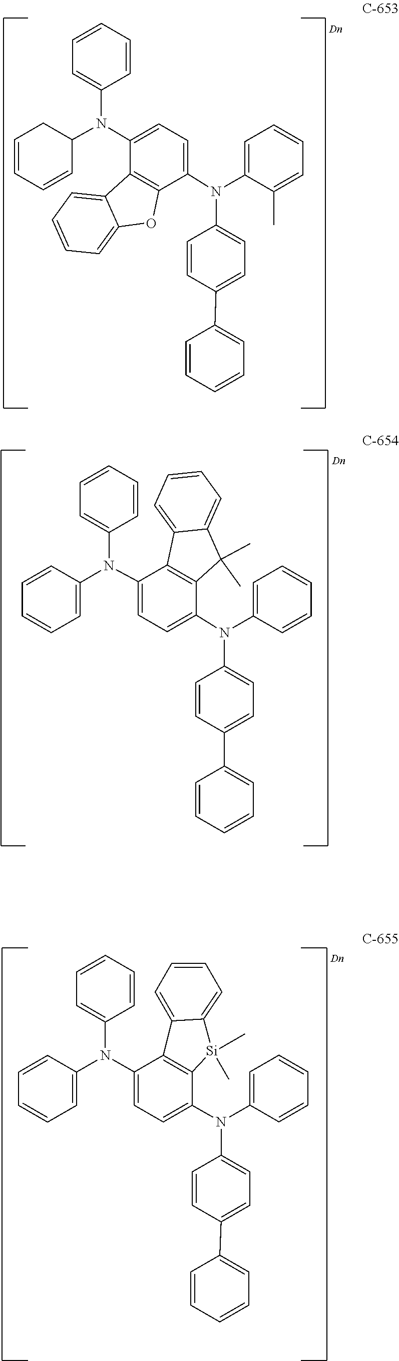 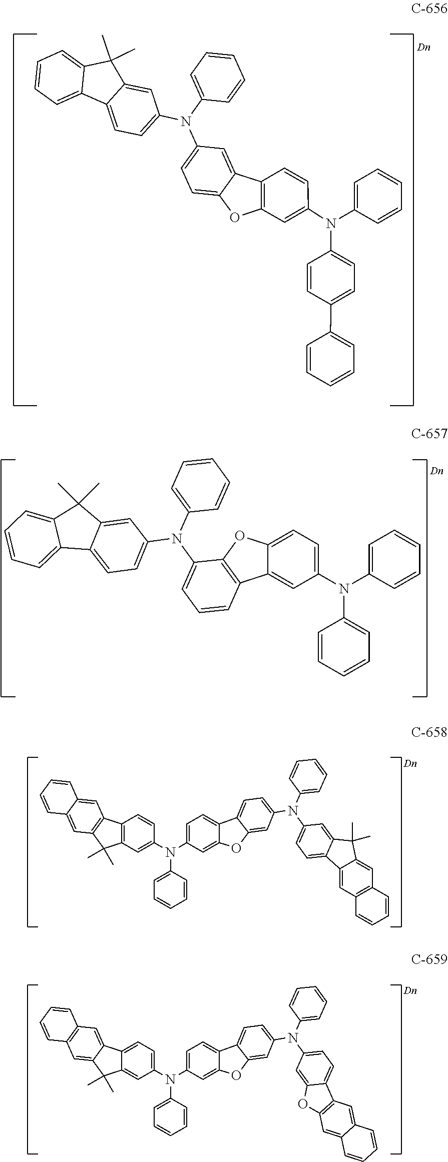 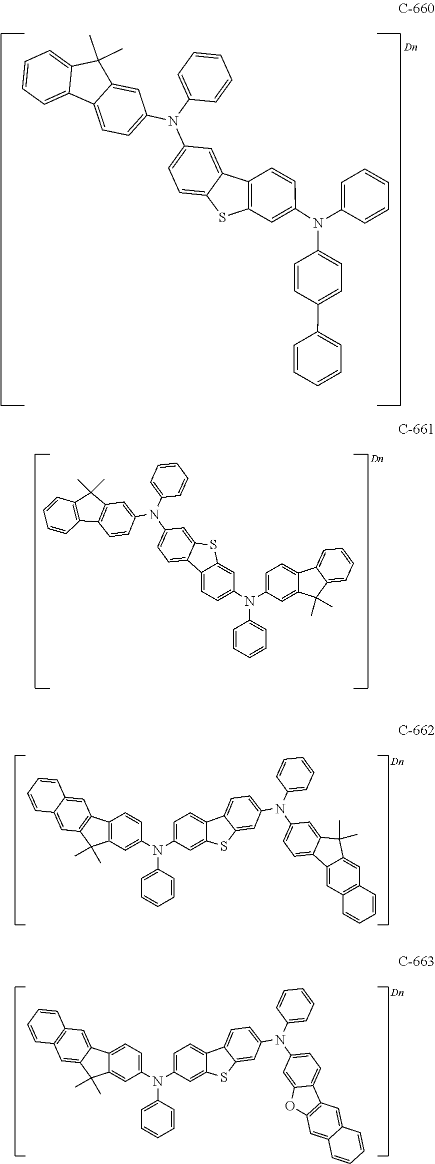 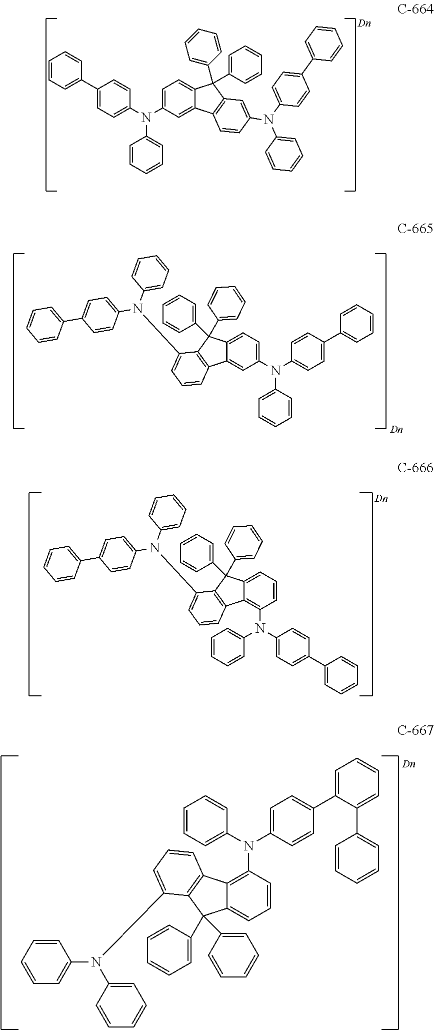 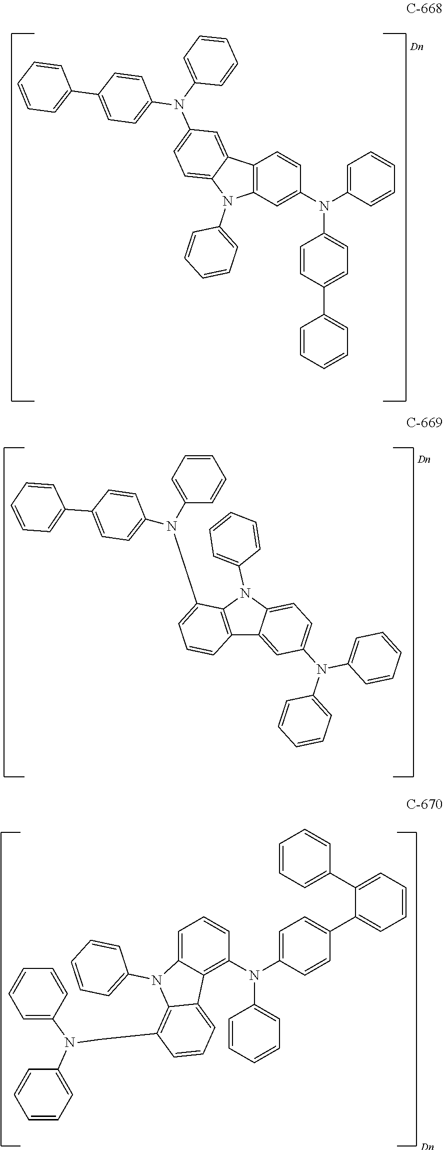 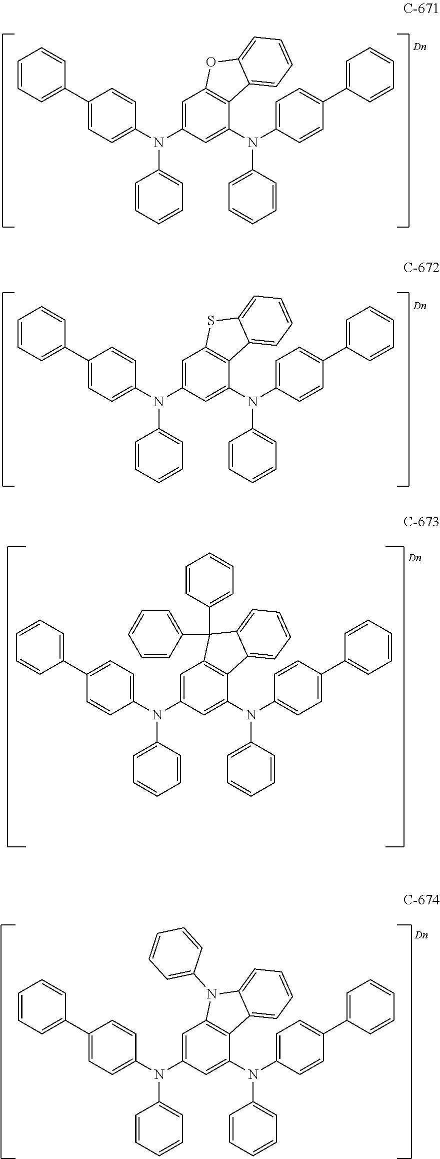 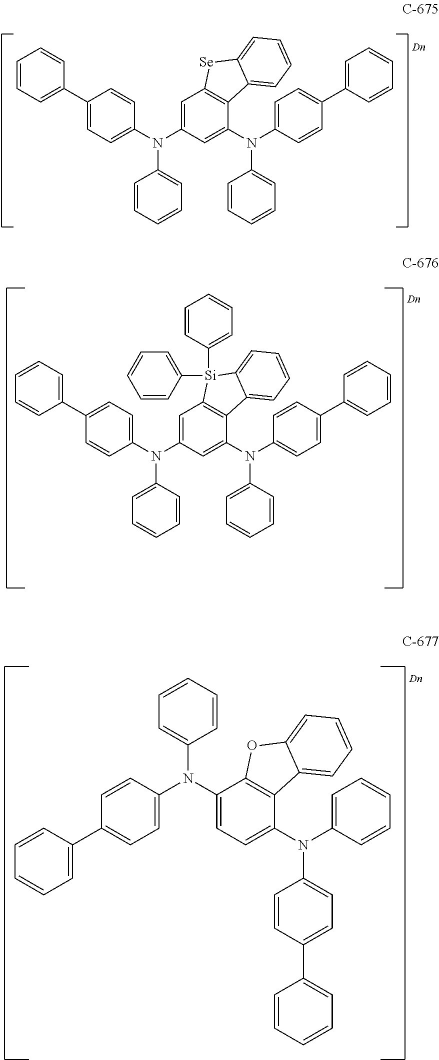 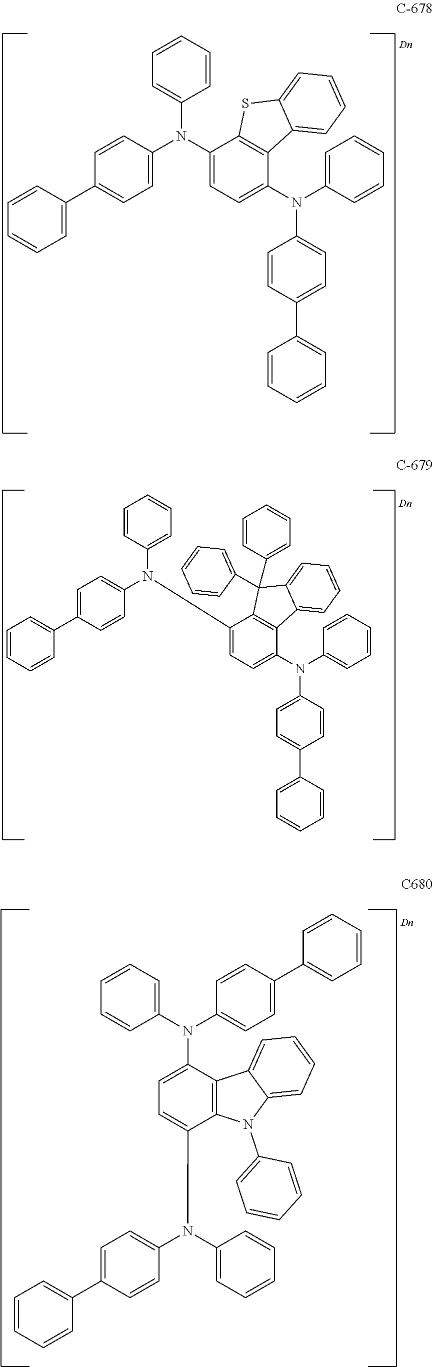 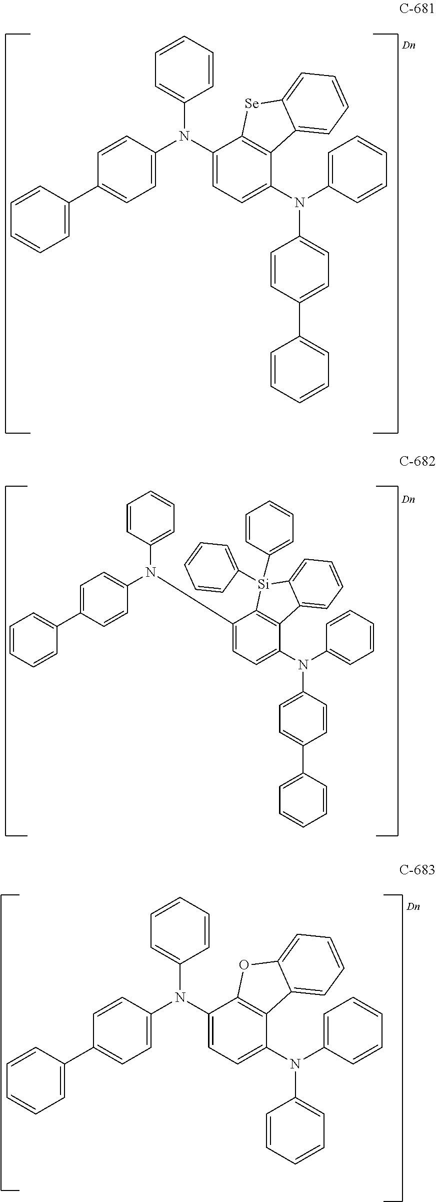 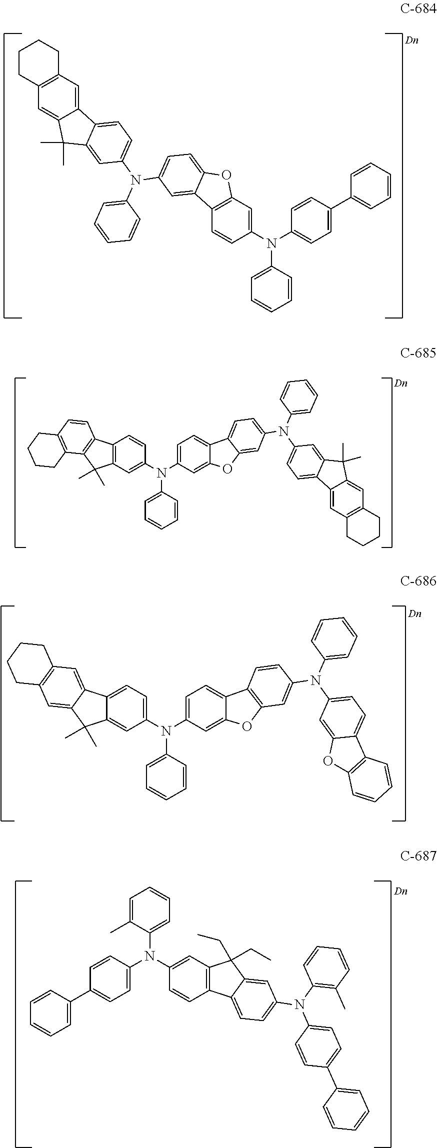 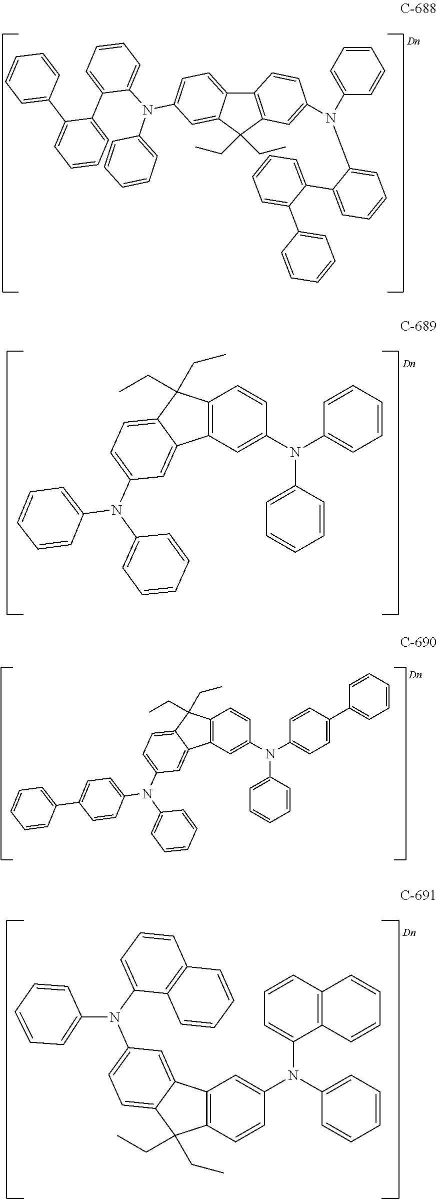 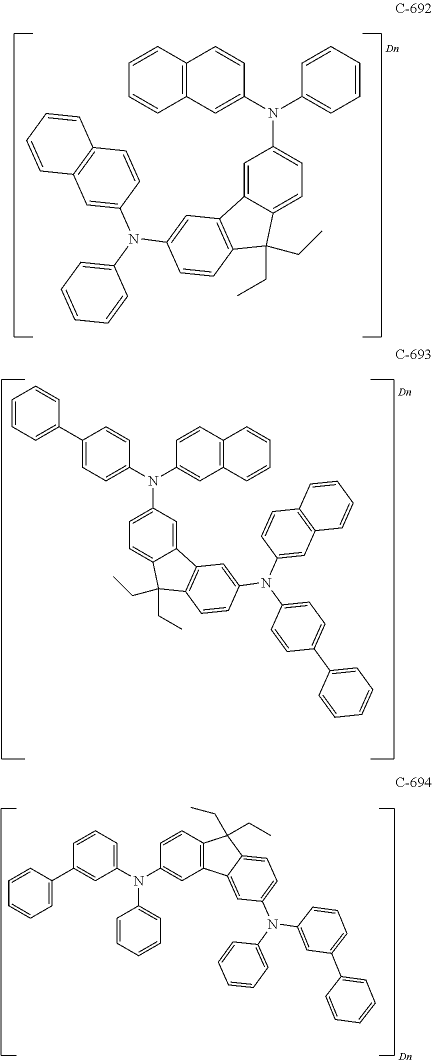 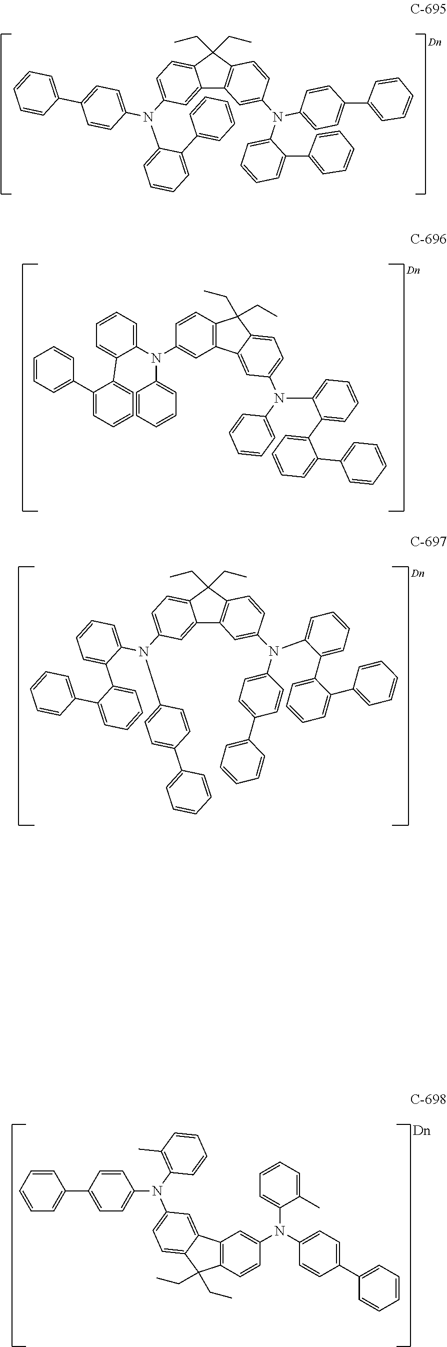 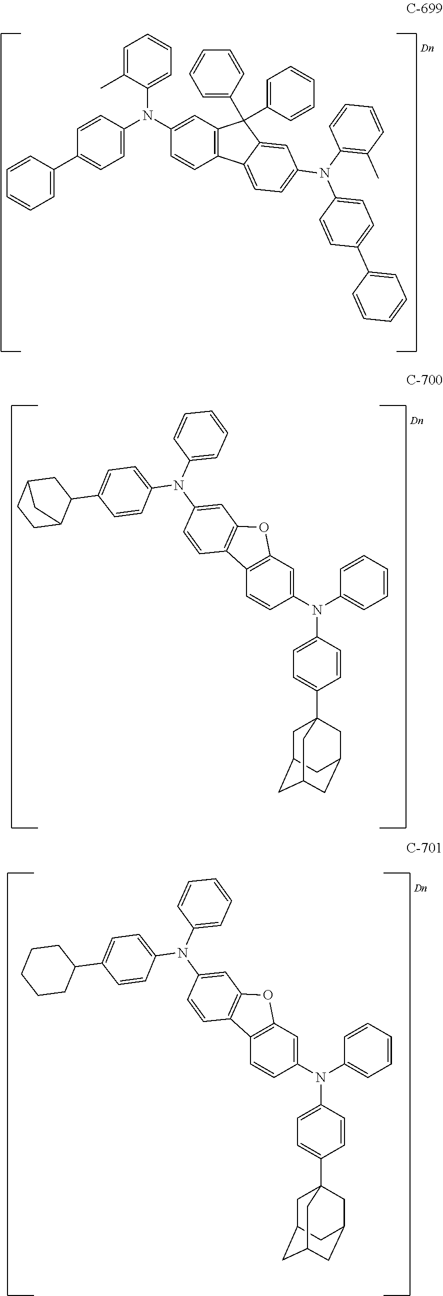 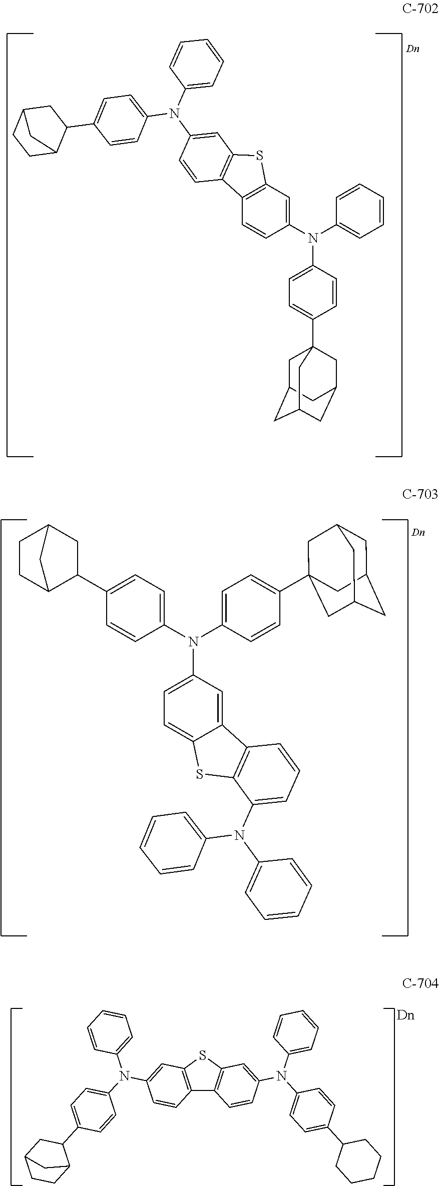 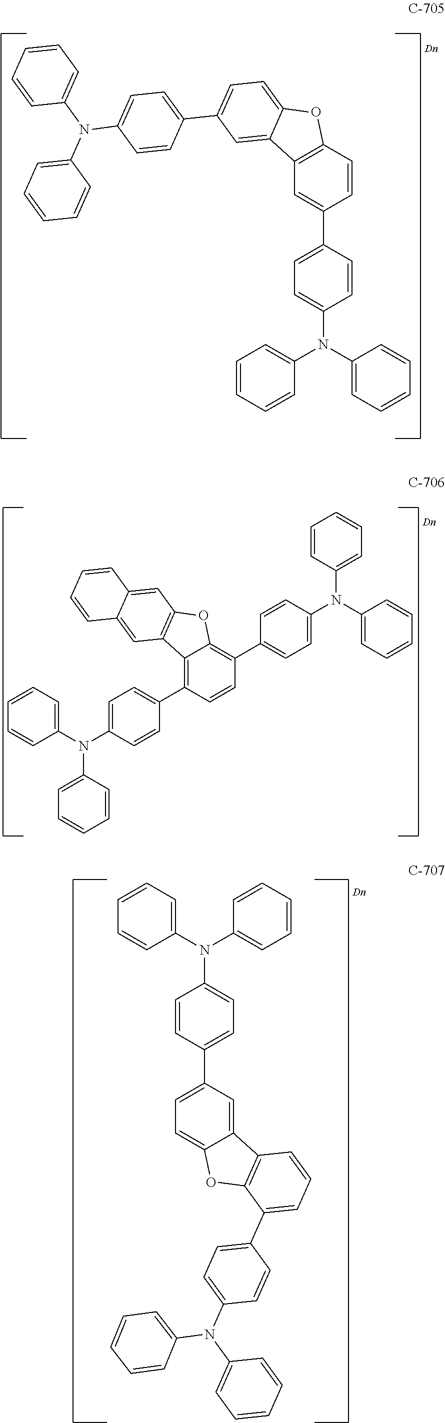 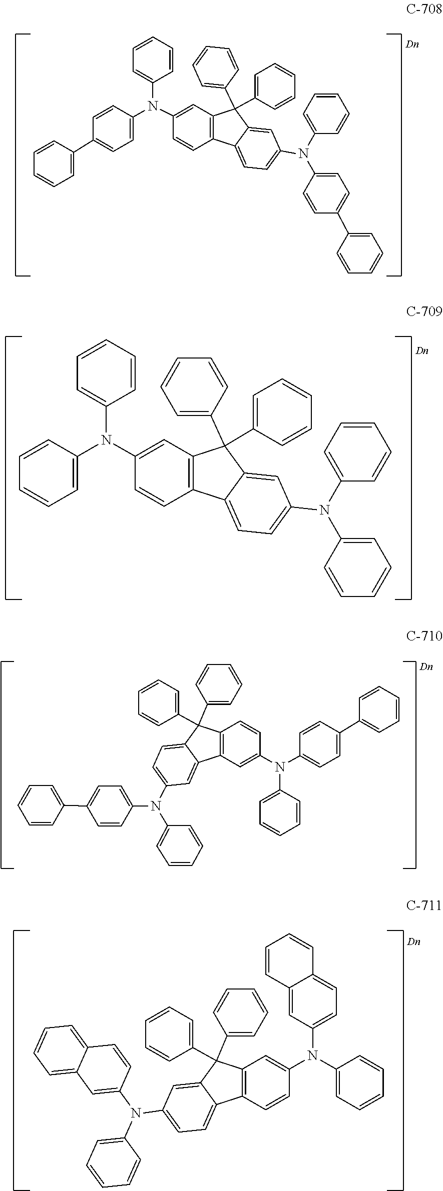 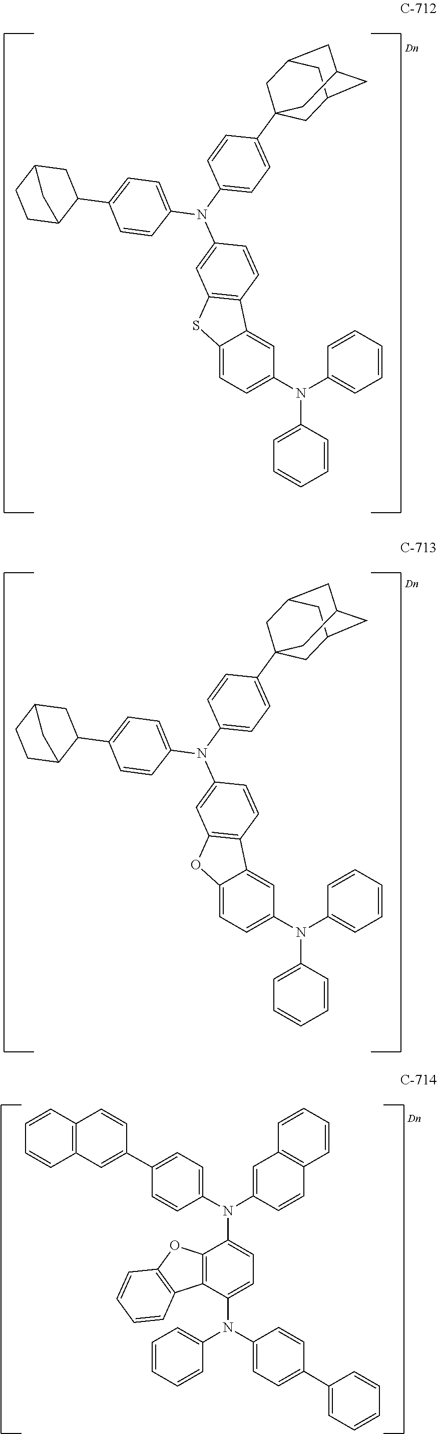 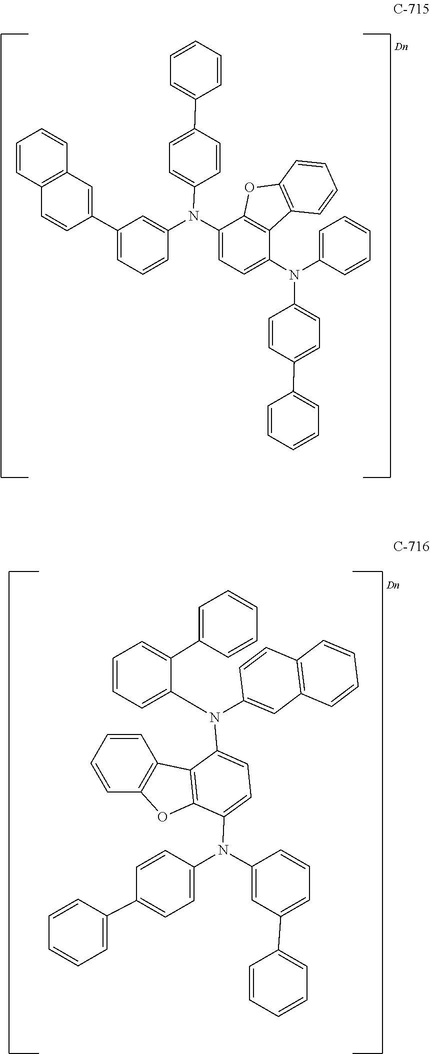 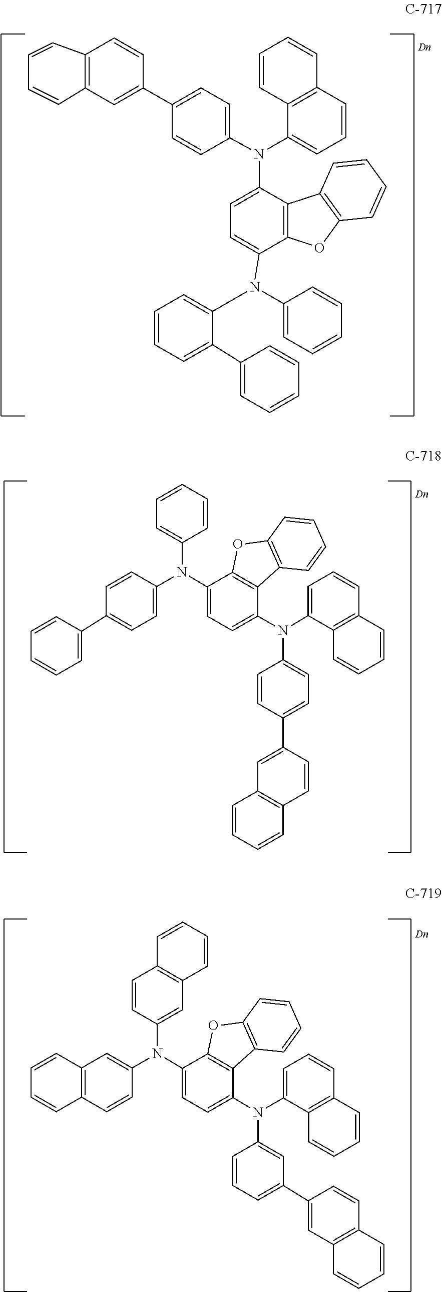 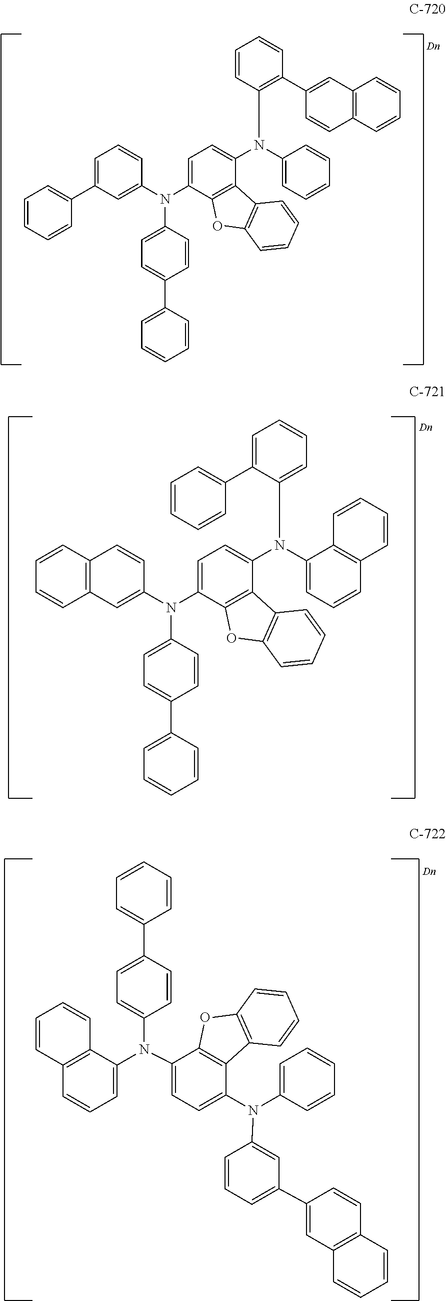 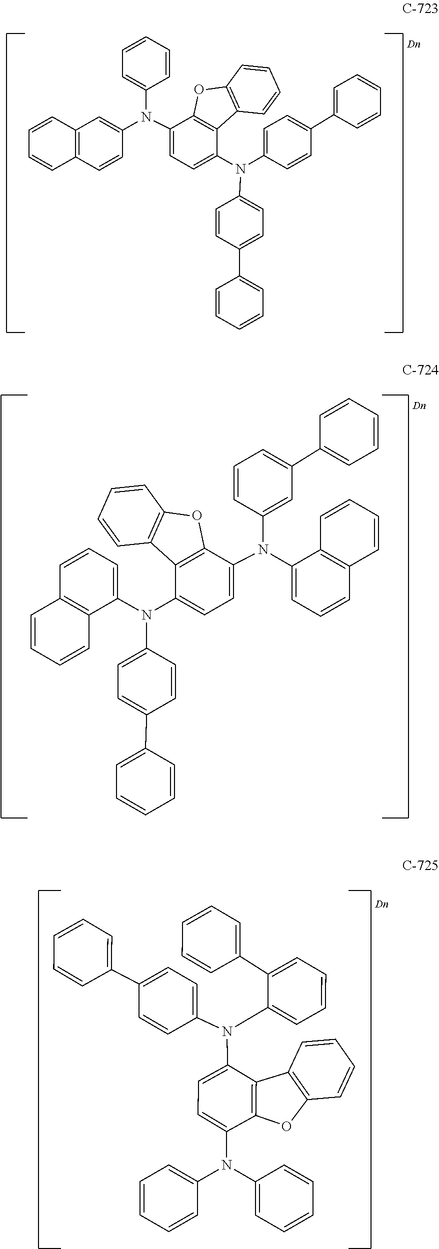 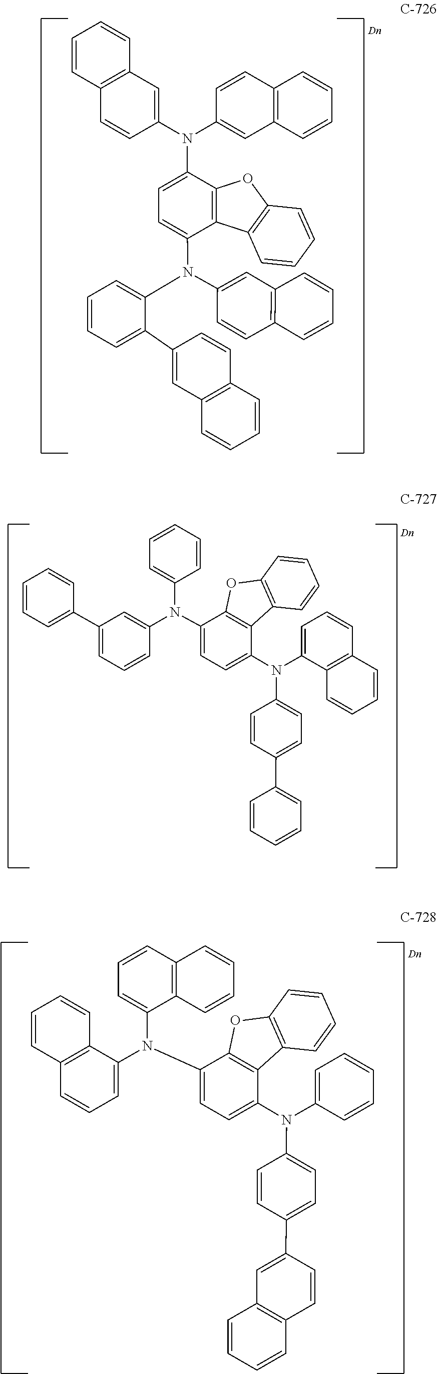 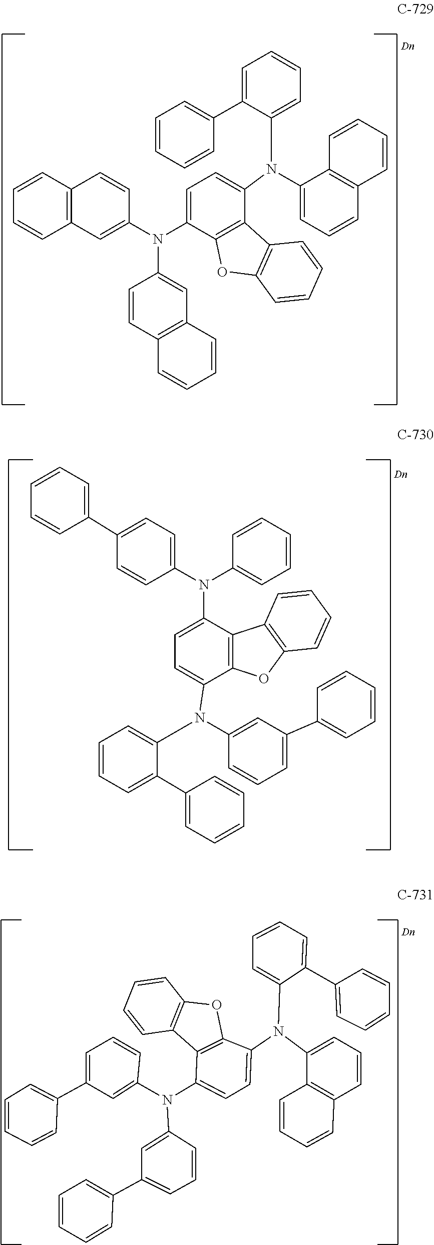 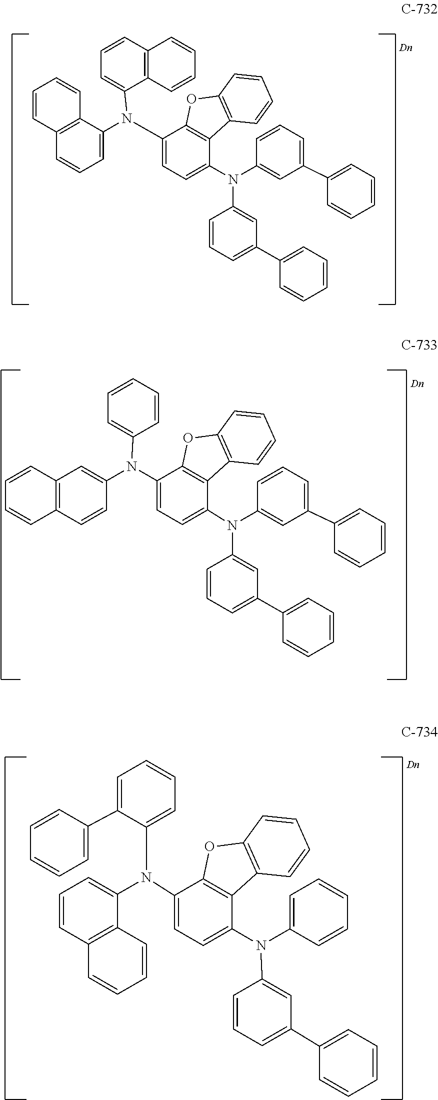 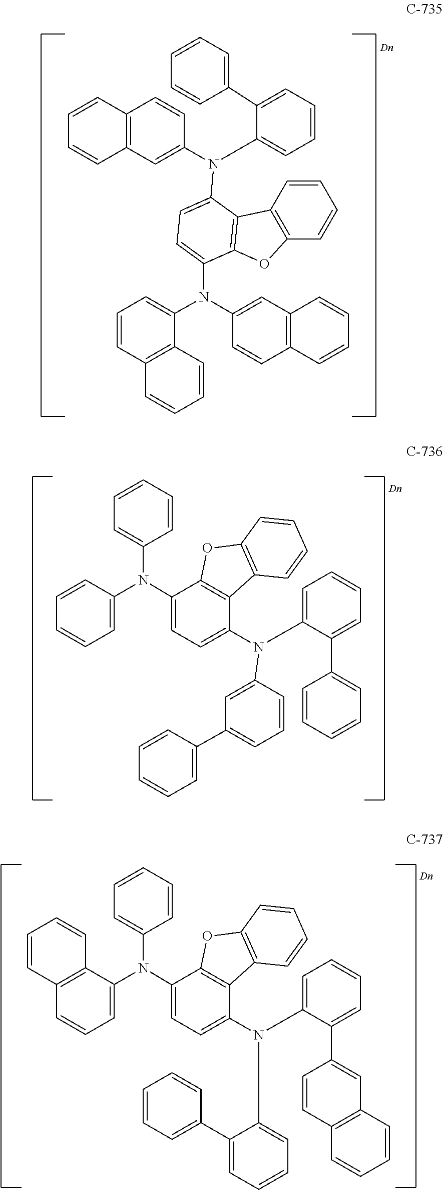 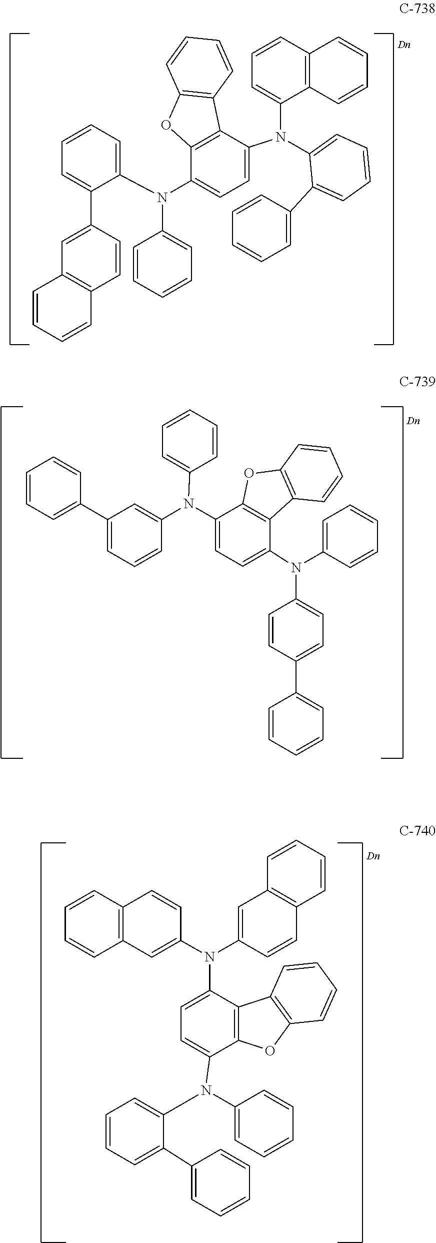 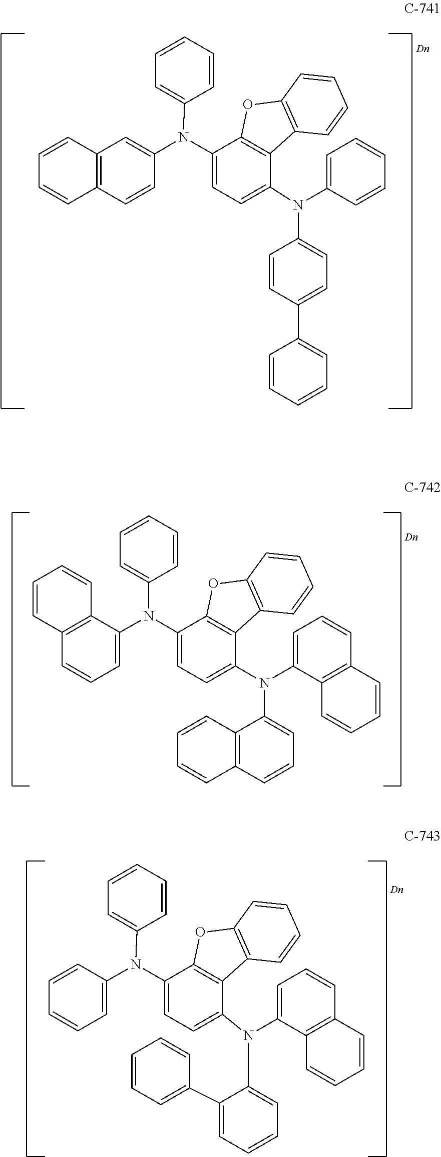 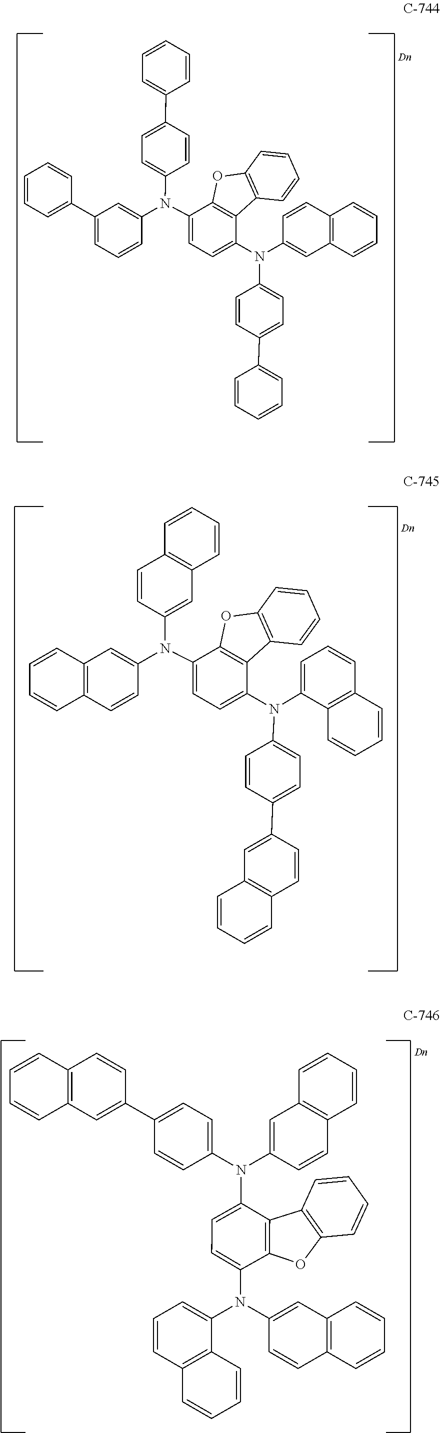 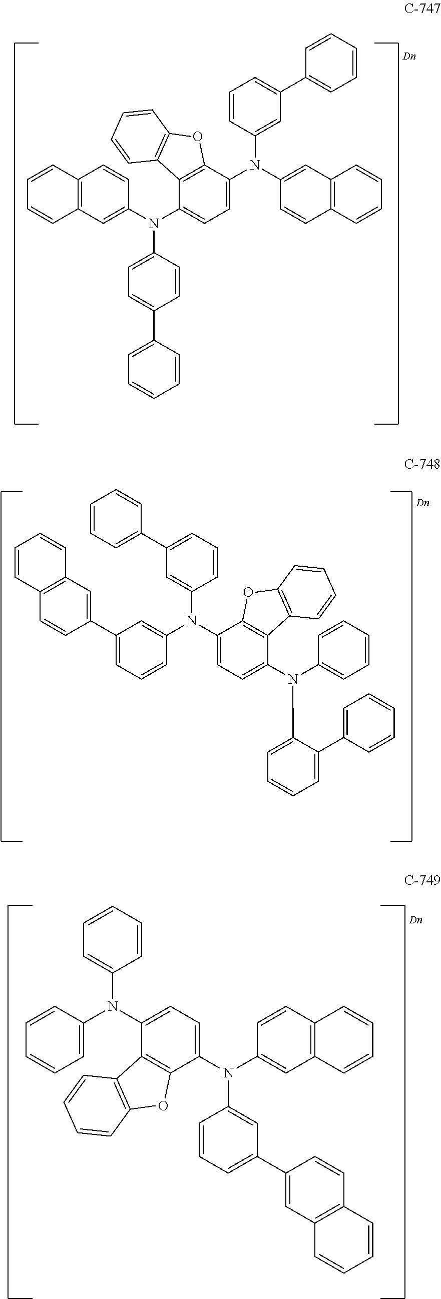 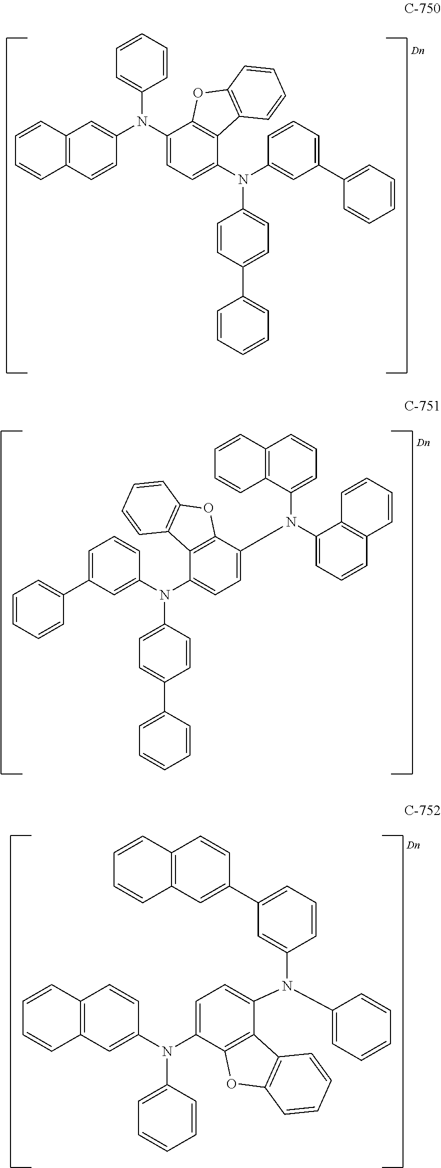 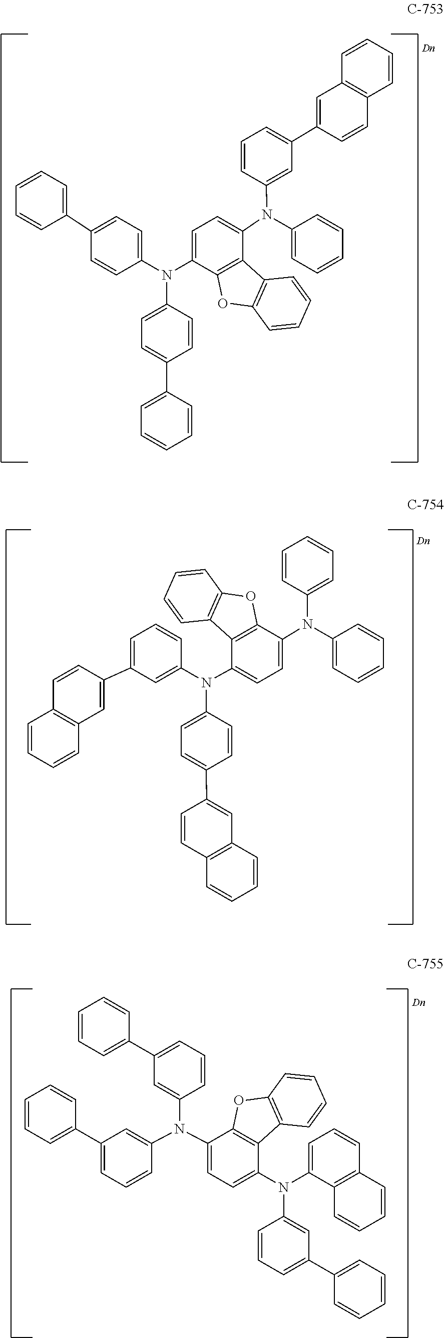 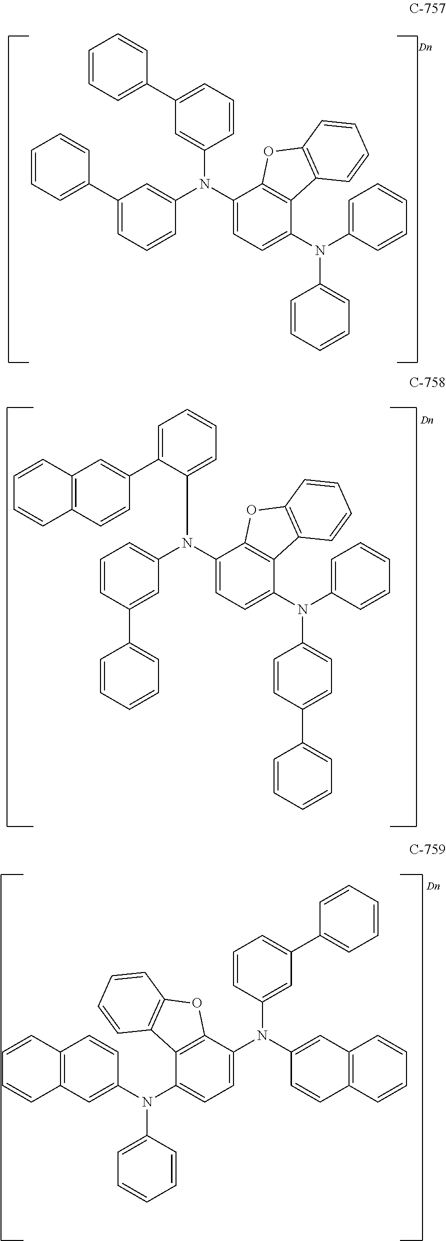 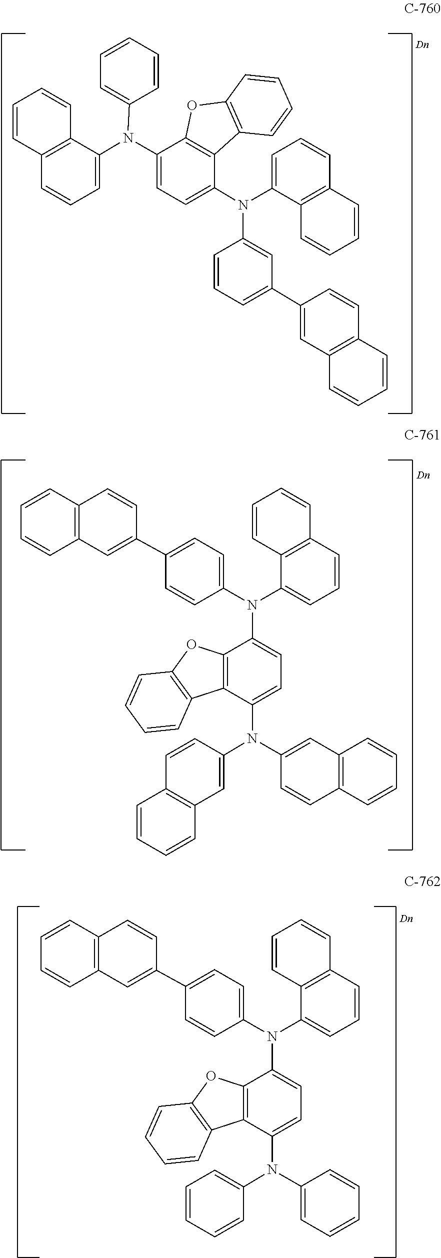 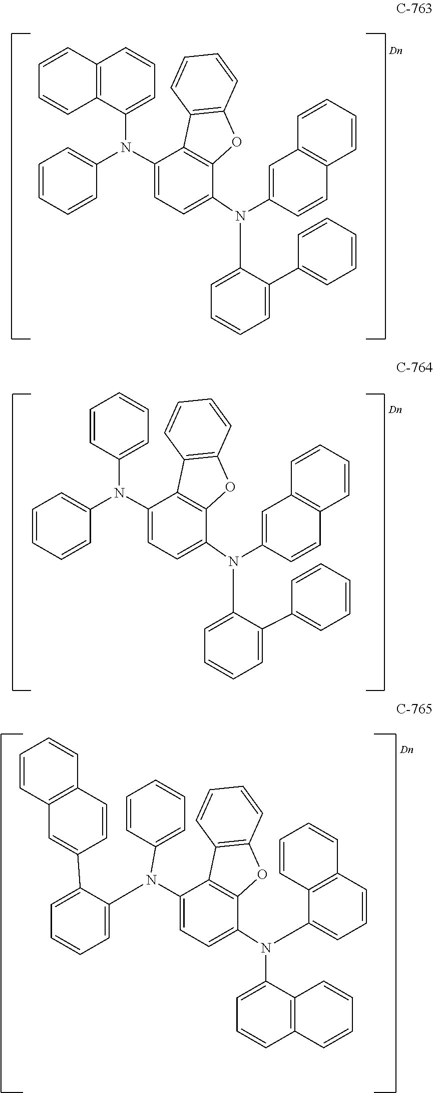 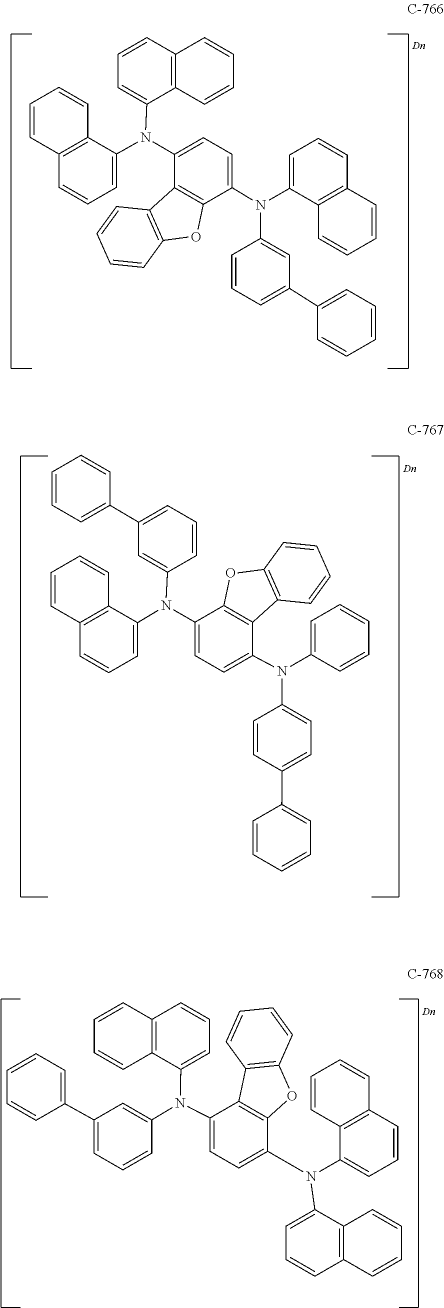 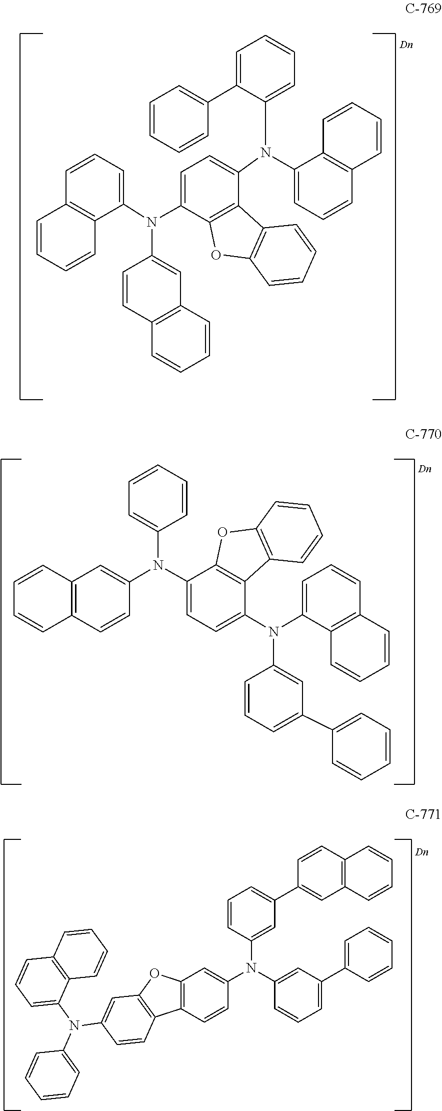 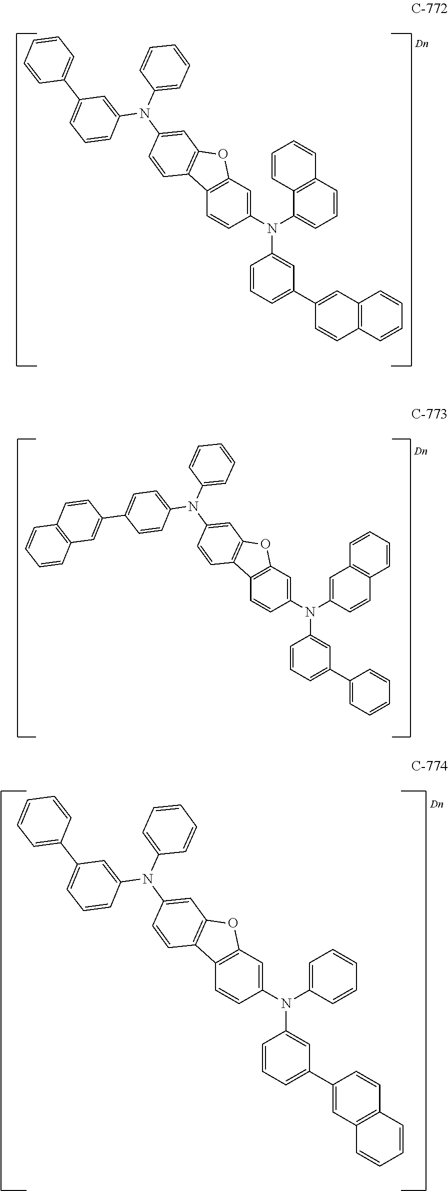 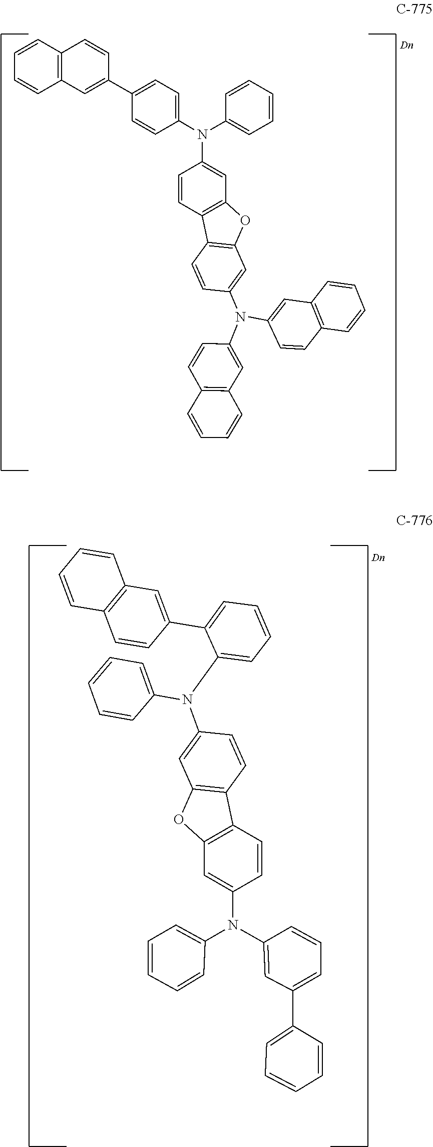 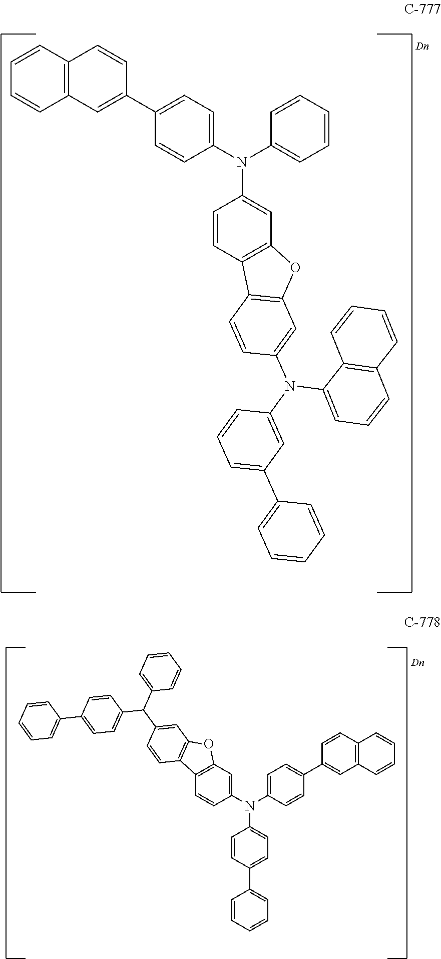 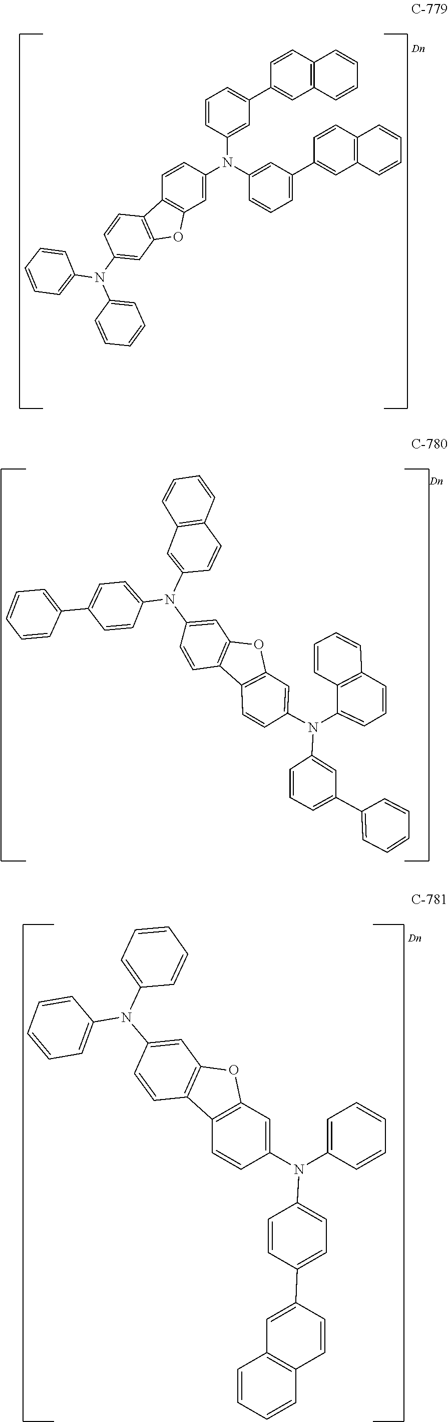 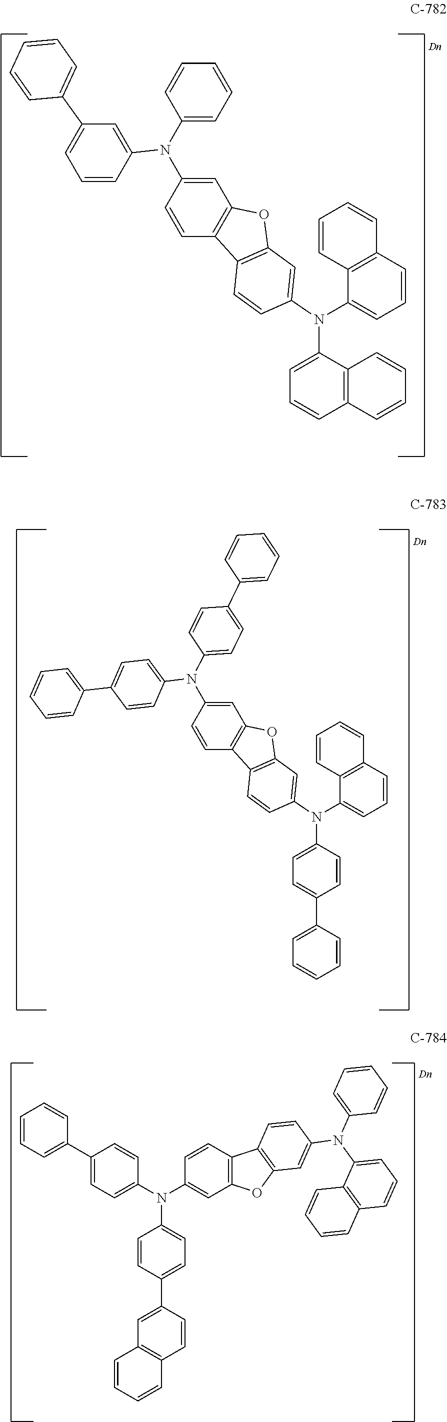 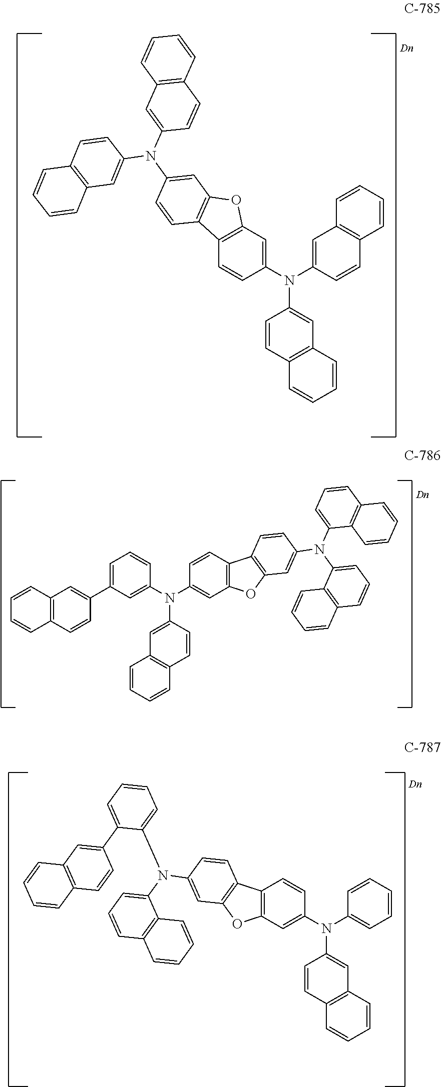 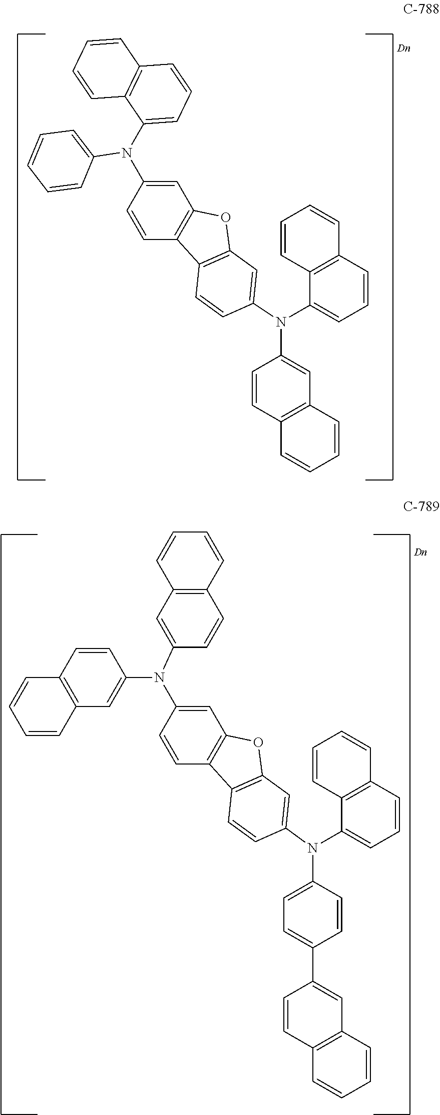 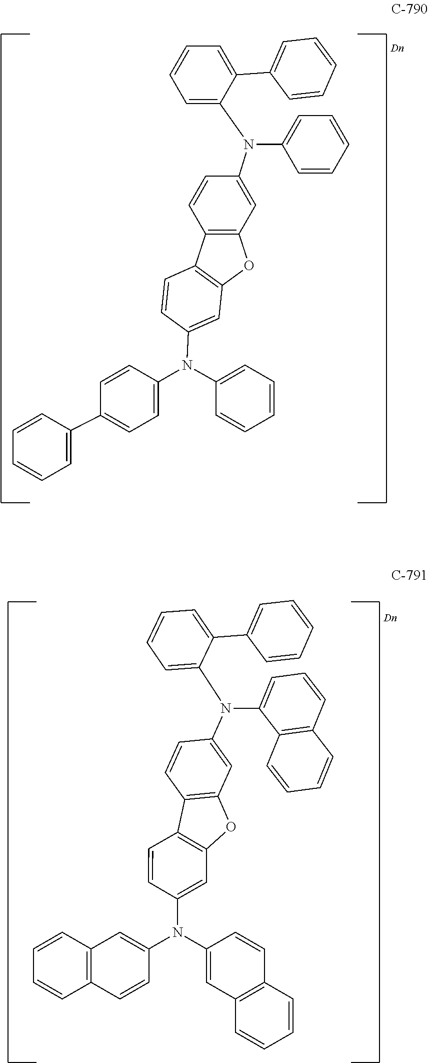 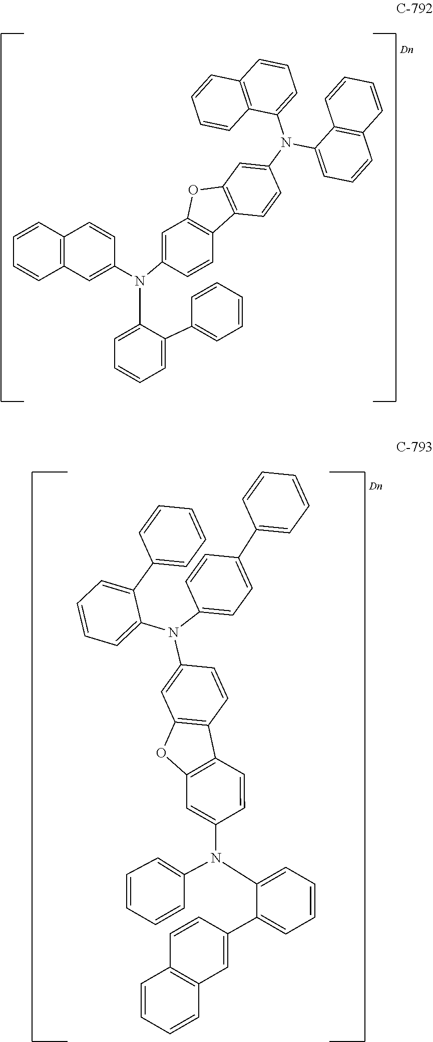 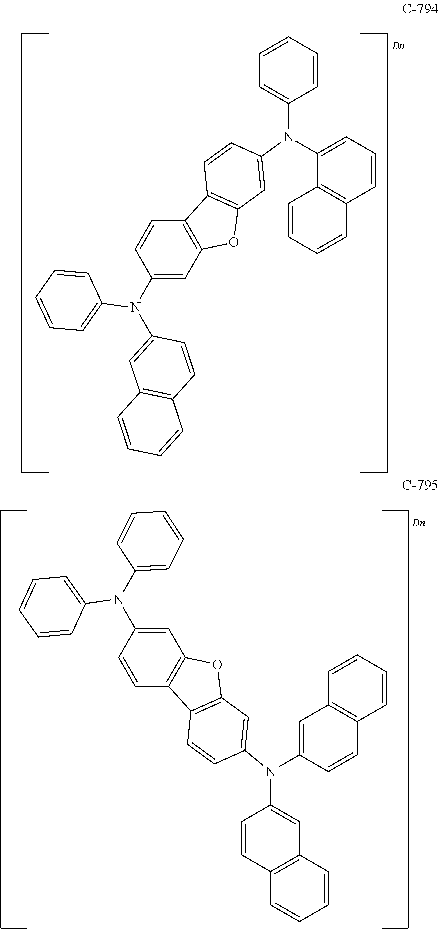 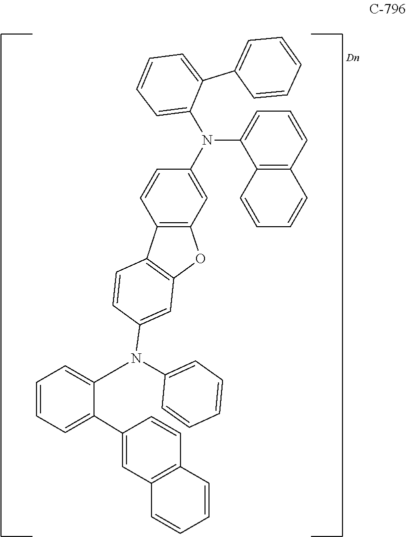 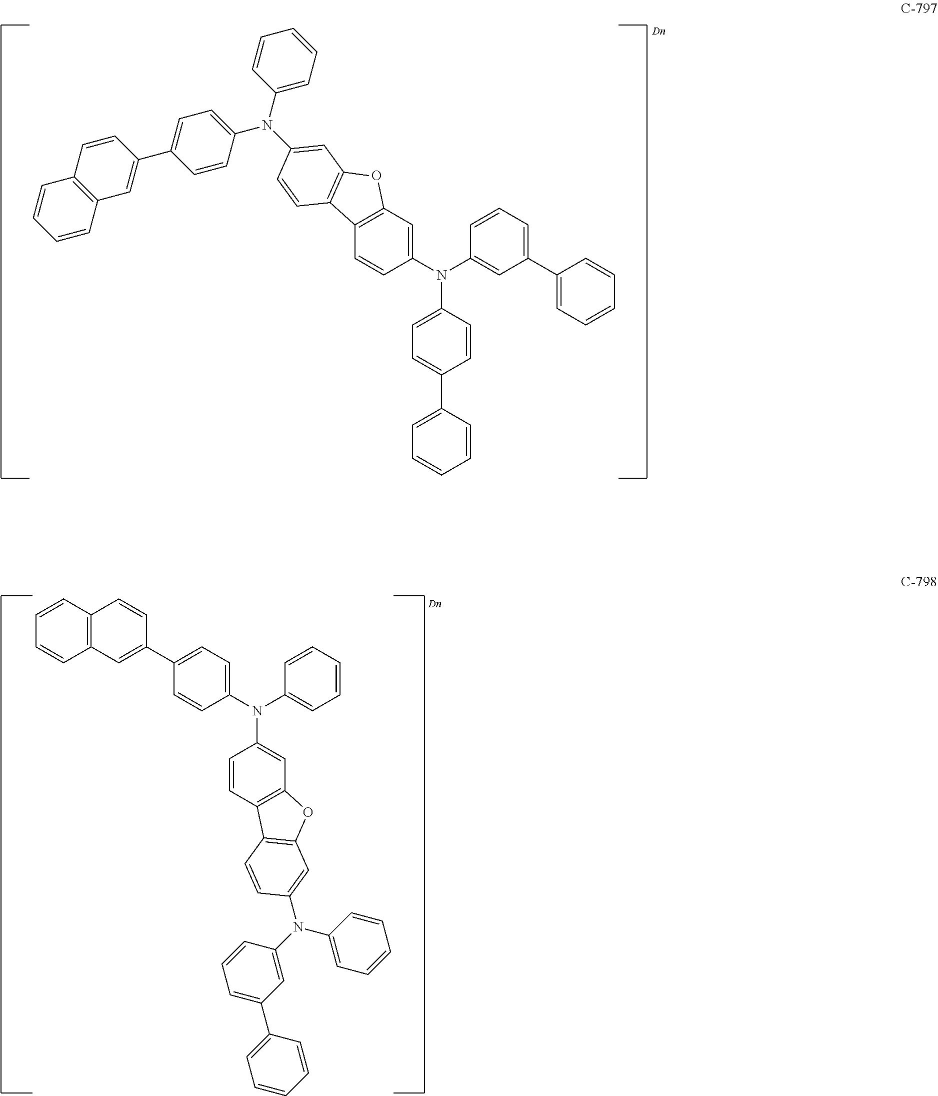 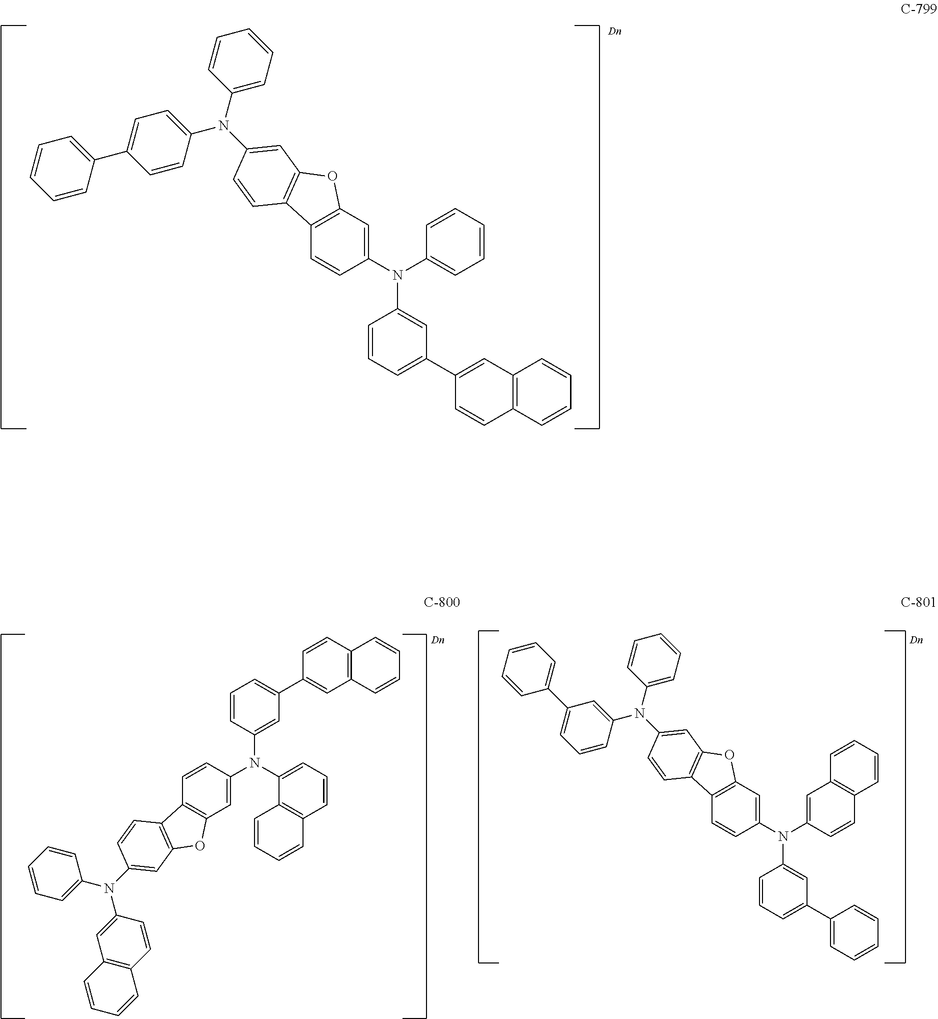 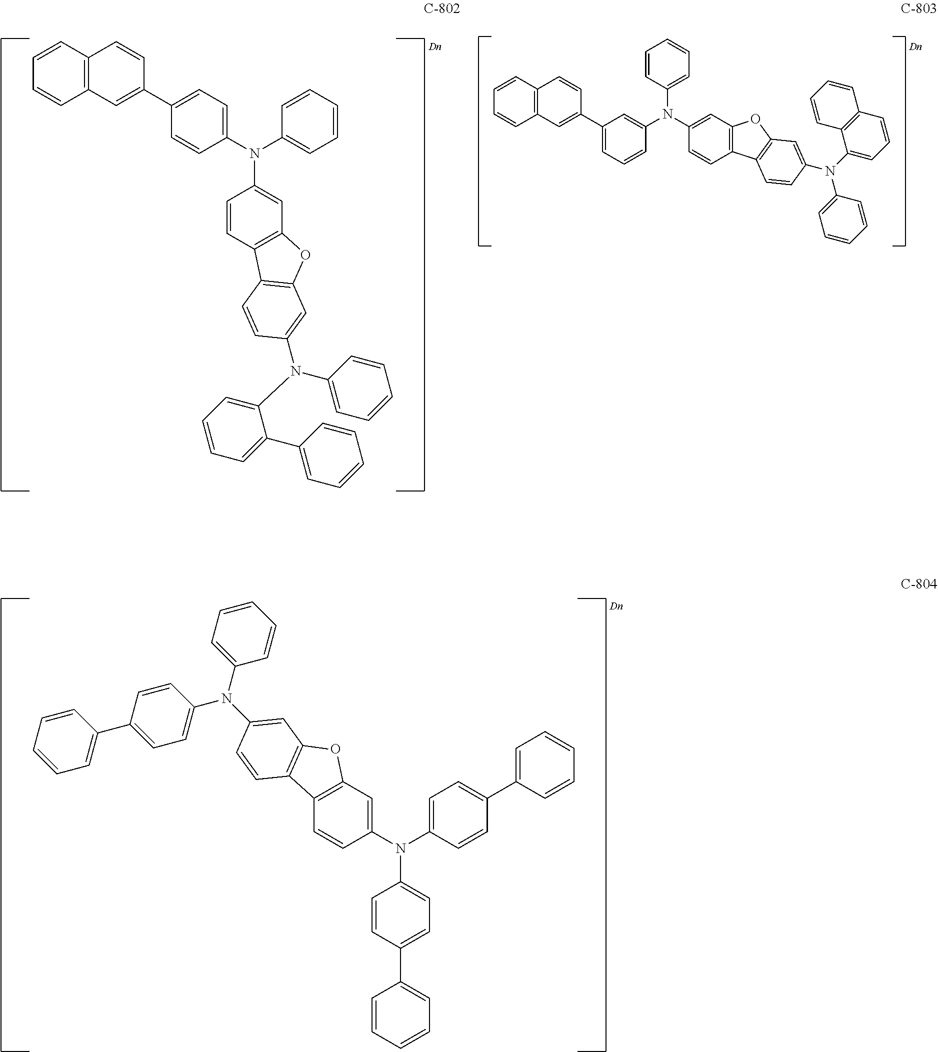 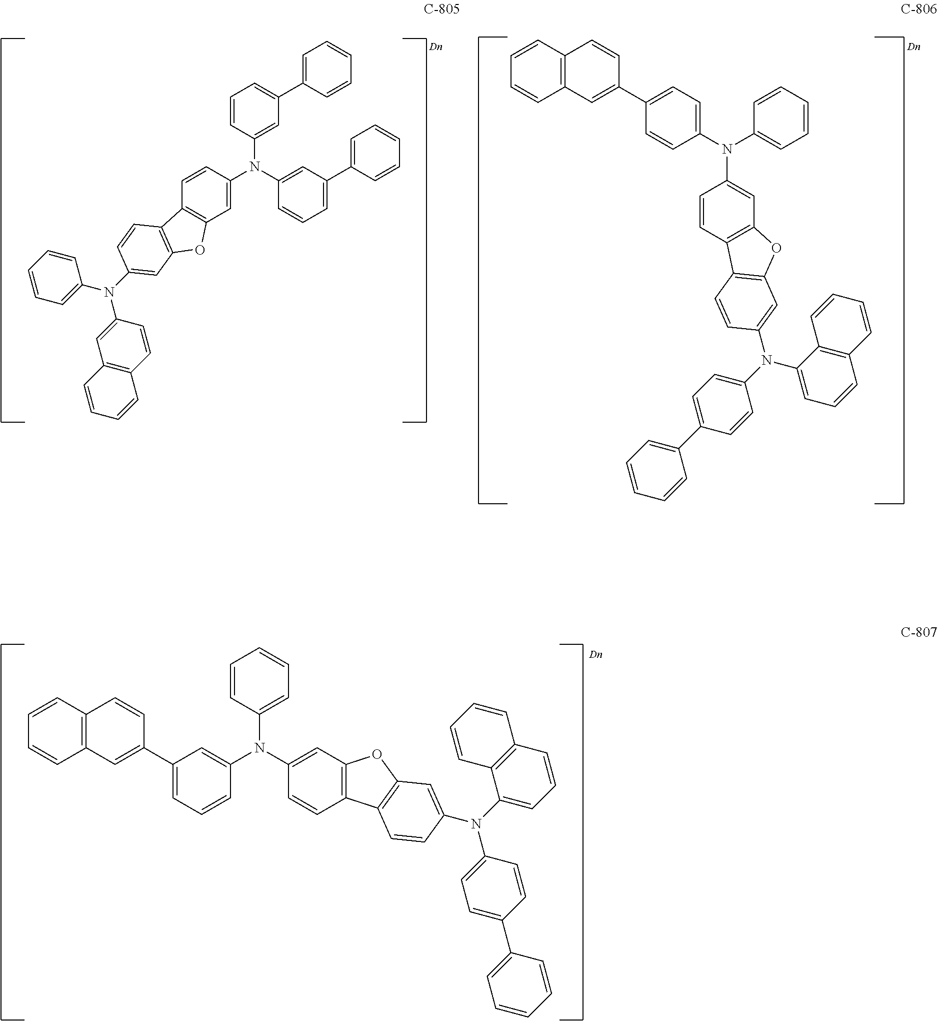 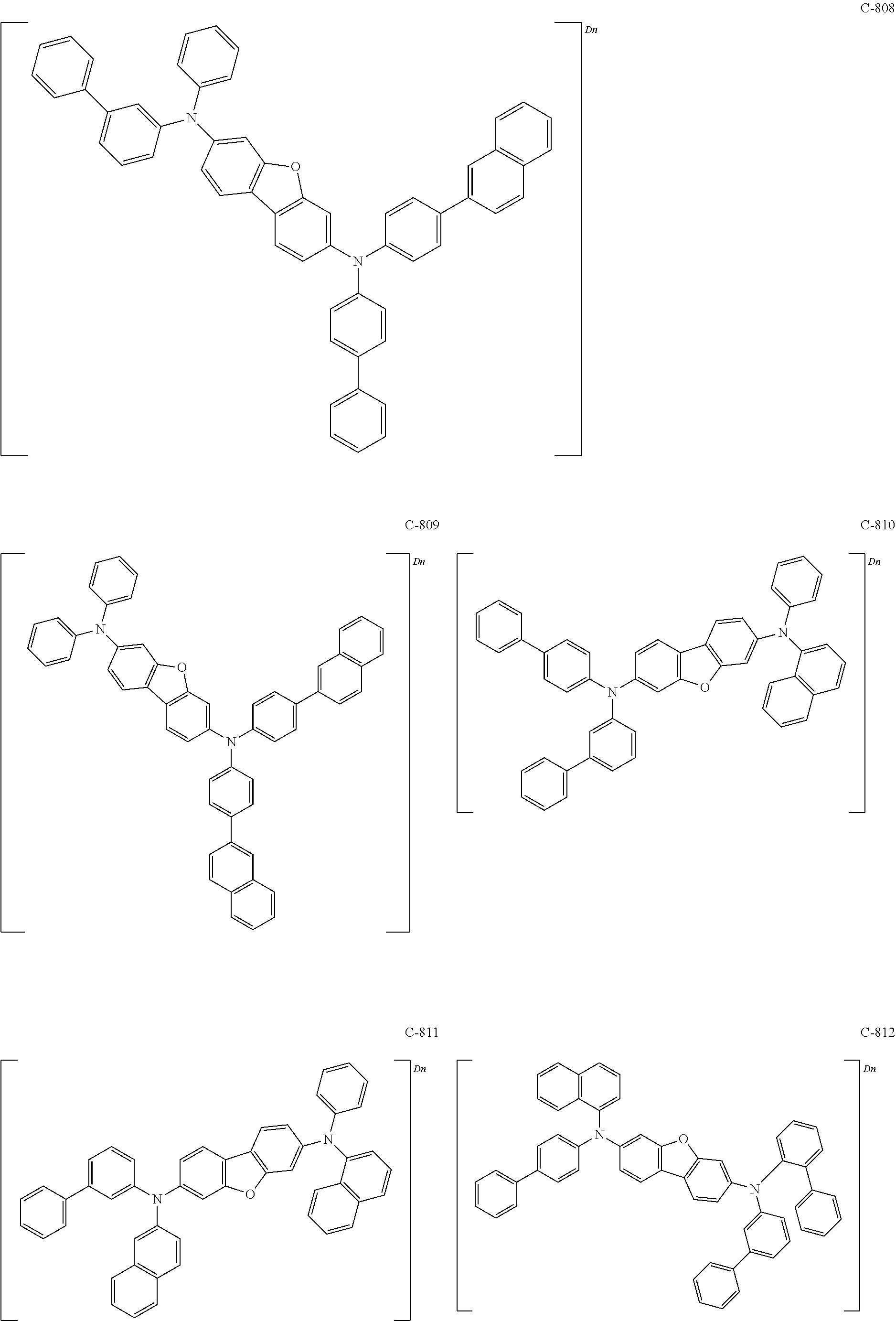 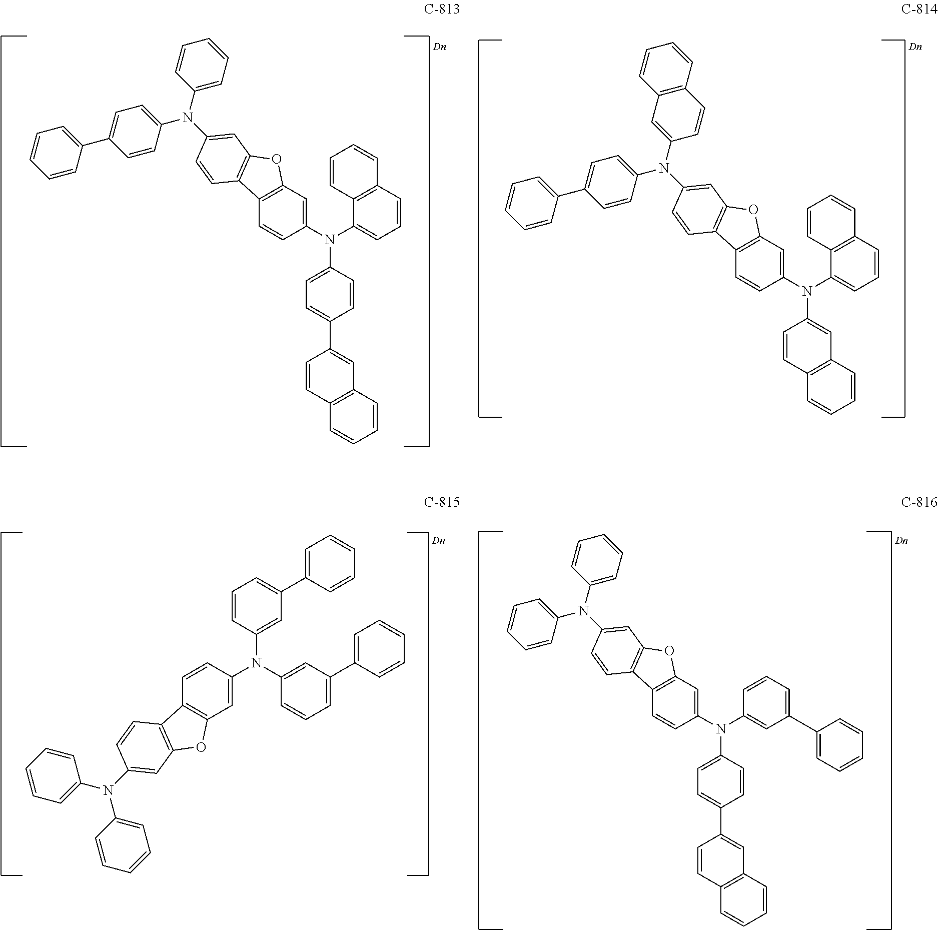 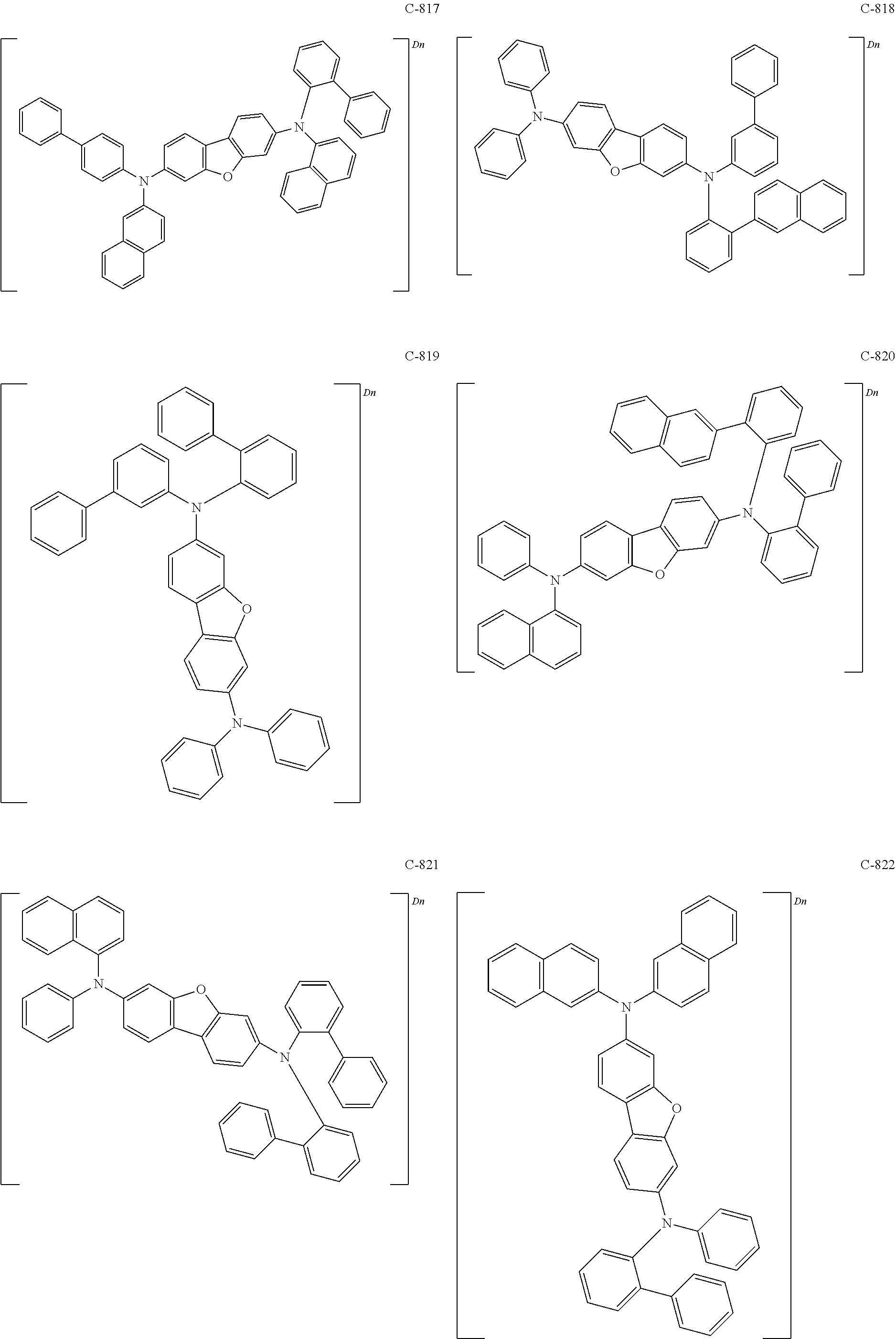 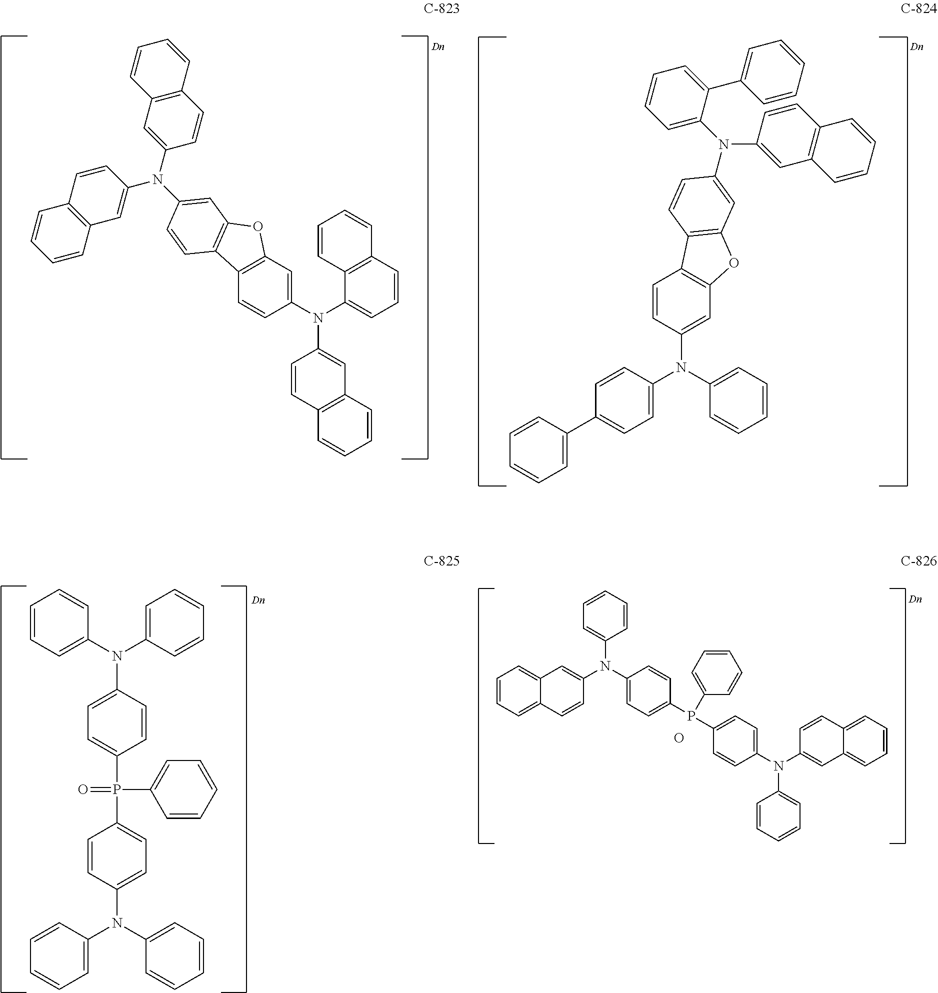 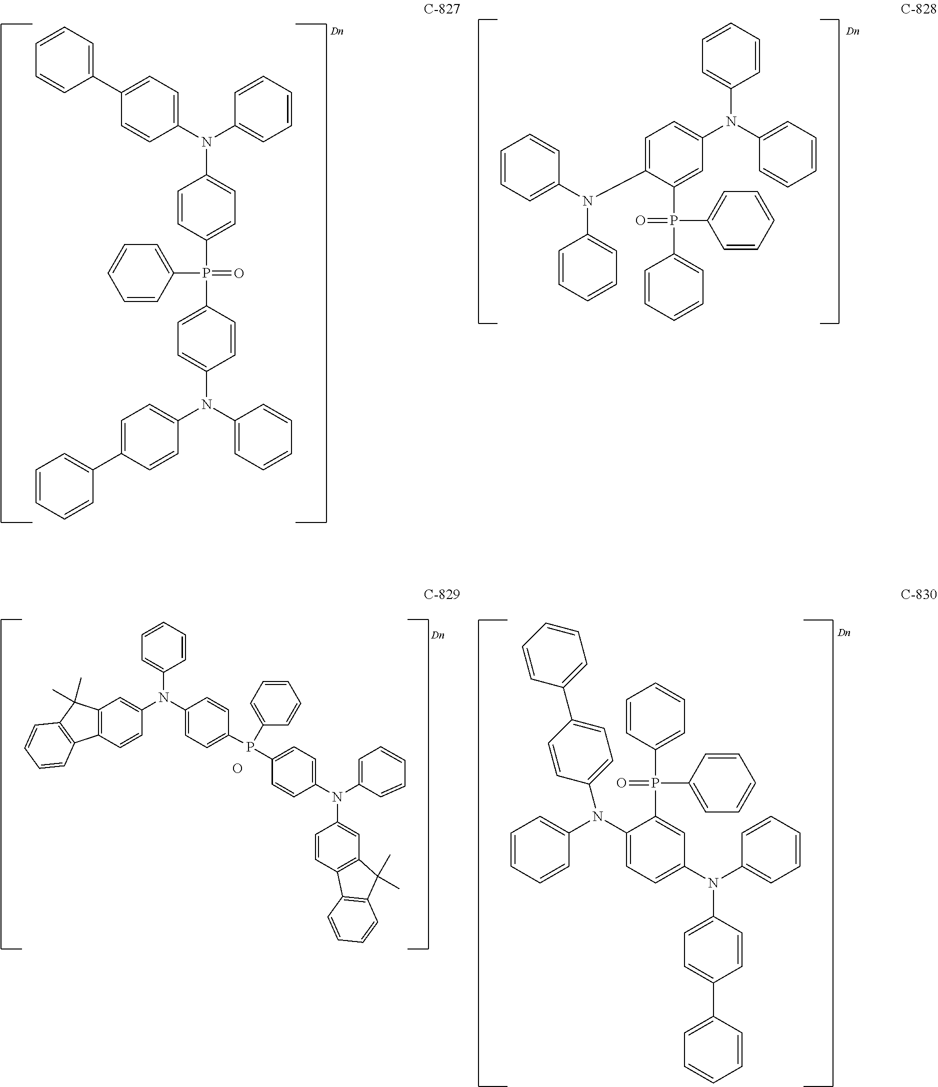 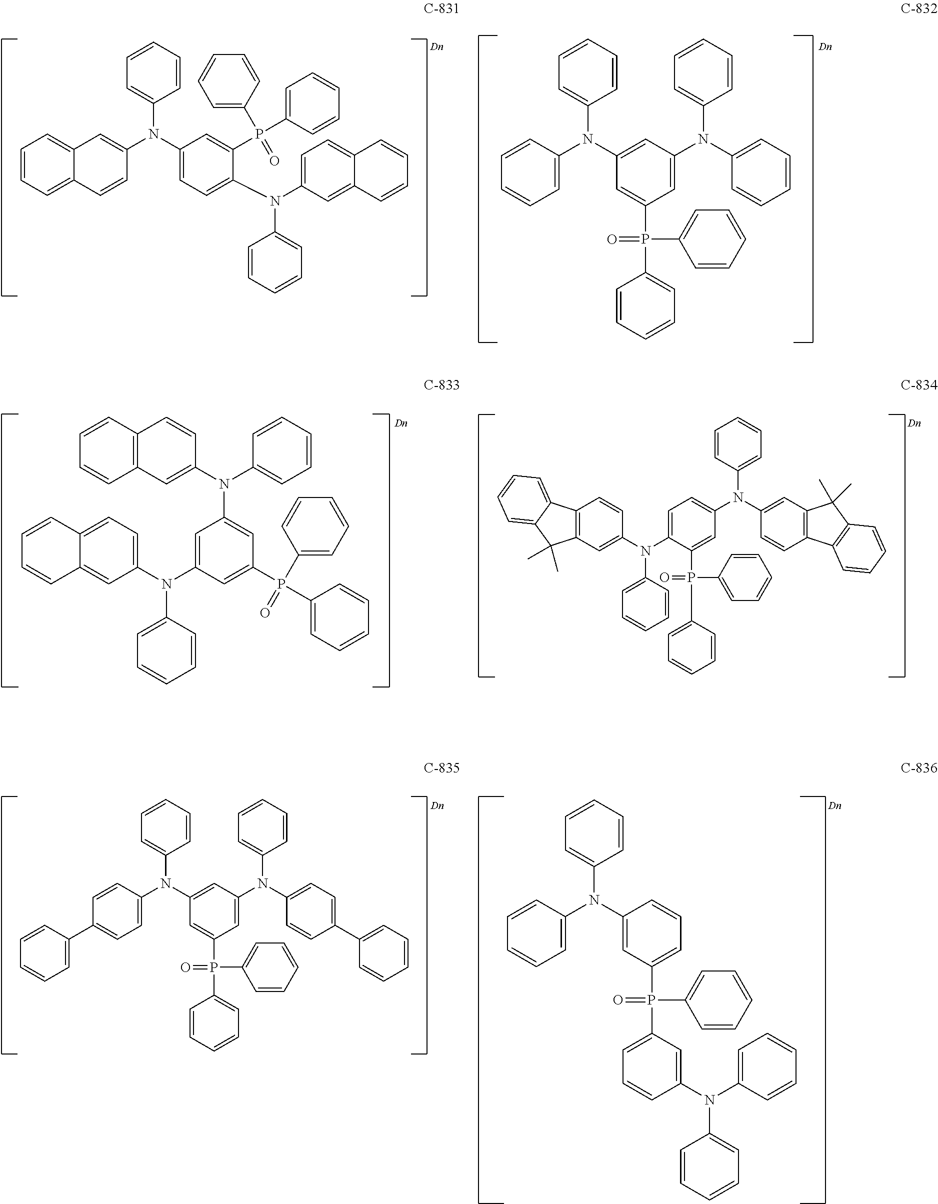 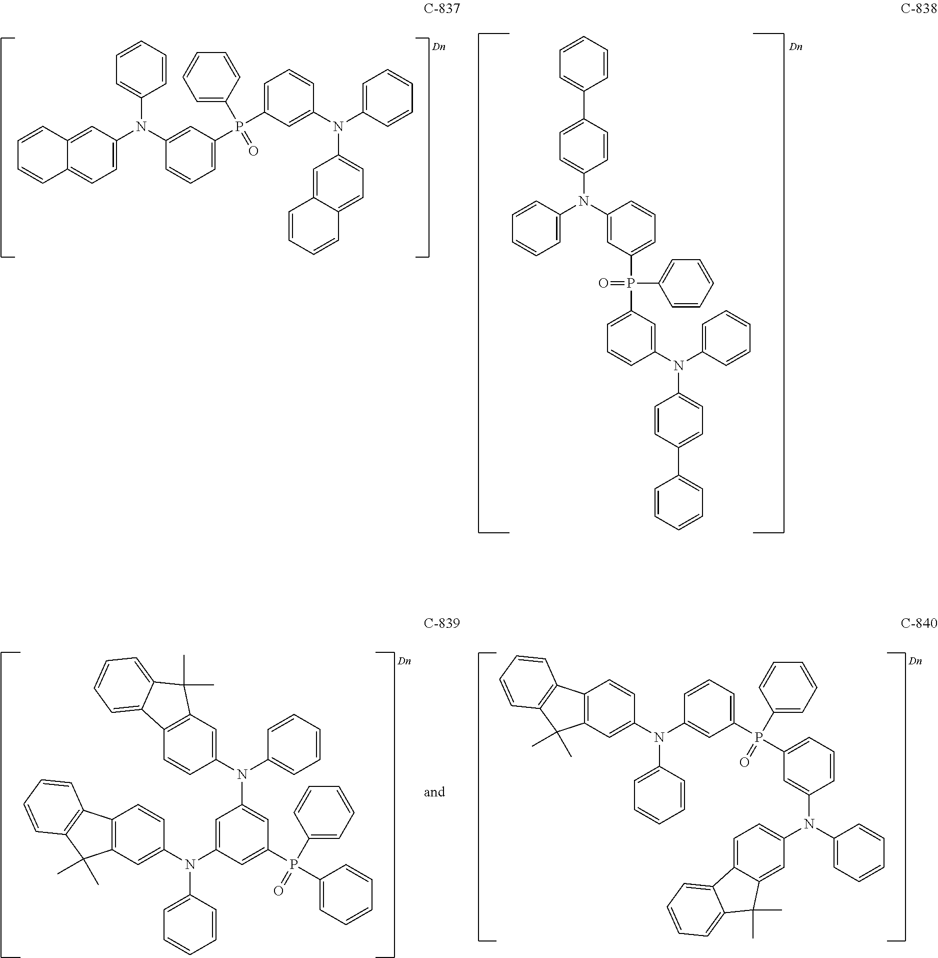
wherein in the compounds above, Dₙ means that n number of hydrogens are replaced with deuterium, and n represents an integer from 1 to the maximum number of hydrogens in the compound.

13. The organic electroluminescent device according to claim 1, wherein the organic layer further comprises a first stack and a second stack, wherein a charge generation region is comprised between the first stack and the second stack, and wherein the charge generation region comprises an N-type charge-generation layer, and a P-type charge-generation layer.

14. The organic electroluminescent device according to claim 13, wherein the N-type charge-generation layer comprises a compound having a structure comprising at least one of a phenanthroline and a terpyridine, and wherein the N-type charge-generation layer is doped with Li or Yb.

15. An organic electroluminescent material comprising a compound represented by the following Formula 1, for use in a hole injection layer: 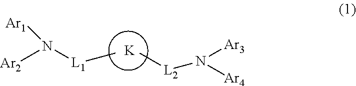
wherein in Formula 1,
ring K is selected from the following Formulas 1-1 to 1-4, 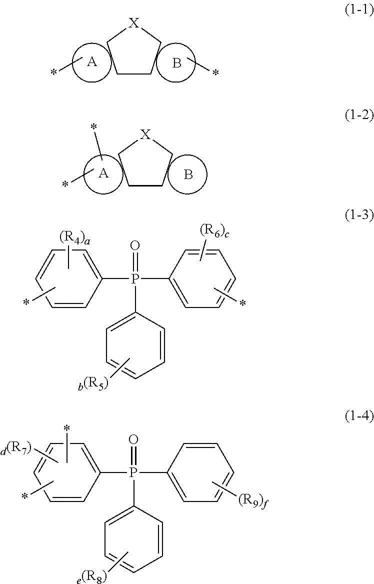
wherein in Formulas 1, and 1-1 to 1-4,
X represents —N(R₁)—, —O—, —S—, —C(R₂)(R₃)—, —Se—, —Si(R₂)(R₃)—, or —P(═O)—,
ring A and ring B each independently represent a substituted or unsubstituted (C₆-C₃₀)arene, or a substituted or unsubstituted (3- to 30-membered)heteroarene;
R₁ to R₃ each independently represent a substituted or unsubstituted (C₆-C₃₀)aryl, a substituted or unsubstituted (C₁-C₃₀)alkyl, a substituted or unsubstituted (C₃-C₃₀)cycloalkyl, or a substituted or unsubstituted (3- to 30-membered)heteroaryl, or may be linked to an adjacent substituent(s) to form a substituted or unsubstituted (C₃-C₃₀) mono- or polycyclic, alicyclic, aromatic ring, or a combination thereof, wherein the carbon atoms of the formed ring may be replaced with at least one heteroatom selected from nitrogen, oxygen, and sulfur;
R₄ to R₉ and Ar₁ to Ar₄ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀)aliphatic ring(s) and a (C₆-C₃₀)aromatic ring(s), a substituted or unsubstituted mono- or di(C₁-C₃₀)alkylamino, a substituted or unsubstituted mono- or di(C₂-C₃₀)alkenylamino, a substituted or unsubstituted mono- or di(C₆-C₃₀)arylamino, a substituted or unsubstituted mono- or di(3- to 30-membered)heteroarylamino, a substituted or unsubstituted (C₁-C₃₀)alkyl(C₂-C₃₀)alkenylamino, a substituted or unsubstituted (C₁-C₃₀)alkyl(C₆-C₃₀)arylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, or a substituted or unsubstituted (C₆-C₃₀)aryl(3- to 30-membered)heteroarylamino, or may be linked to an adjacent substituent(s) to form a substituted or unsubstituted (C₃-C₃₀)mono- or polycyclic, alicyclic, aromatic ring, or a combination thereof, wherein the carbon atoms of the formed ring may be replaced with at least one heteroatom selected from nitrogen, oxygen, and sulfur;
L₁ and L₂ each independently represent a single bond, a substituted or unsubstituted (C₆-C₃₀)arylene, or a substituted or unsubstituted (3- to 30-membered)heteroarylene;
a and c each represent an integer of 1 to 4, b, e, and f each represent an integer of 1 to 5, and d represents an integer of 1 to 3; and
the heteroaryl(ene) comprises at least one heteroatom selected from B, N, O, S, Si, Se, and P.

16. The organic electroluminescent material according to claim 15, wherein the ring A and the ring B are each independently selected from the following Formulas 1-5 to 1-9: 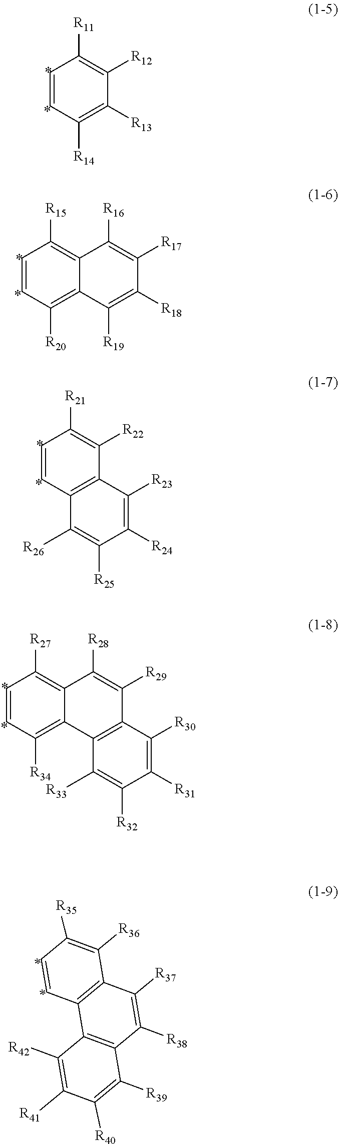
wherein in Formulas 1-5 to 1-9,
R₁₁ to R₄₂ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀)aliphatic ring(s) and a (C₆-C₃₀)aromatic ring(s), a substituted or unsubstituted mono- or di(C₁-C₃₀)alkylamino, a substituted or unsubstituted mono- or di(C₂-C₃₀)alkenylamino, a substituted or unsubstituted mono- or di(C₆-C₃₀)arylamino, a substituted or unsubstituted mono- or di(3- to 30-membered)heteroarylamino, a substituted or unsubstituted (C₁-C₃₀)alkyl(C₂-C₃₀)alkenylamino, a substituted or unsubstituted (C₁-C₃₀)alkyl(C₆-C₃₀)arylamino, a substituted or unsubstituted (C₁-C₃₀)alkyl(3- to 30-membered)heteroarylamino, a substituted or unsubstituted (C₂-C₃₀)alkenyl(C₆-C₃₀)arylamino, a substituted or unsubstituted (C₂-C₃₀)alkenyl(3- to 30-membered)heteroarylamino, or a substituted or unsubstituted (C₆-C₃₀)aryl(3- to 30-membered)heteroarylamino, or may be linked to an adjacent substituent(s) to form a substituted or unsubstituted (C₃-C₃₀) mono- or polycyclic, alicyclic, aromatic ring, or a combination thereof, wherein the carbon atoms of the formed ring may be replaced with at least one heteroatom selected from nitrogen, oxygen, and sulfur.

17. The organic electroluminescent material according to claim 15, wherein the compound represented by Formula 1 is at least one selected from the following compounds: 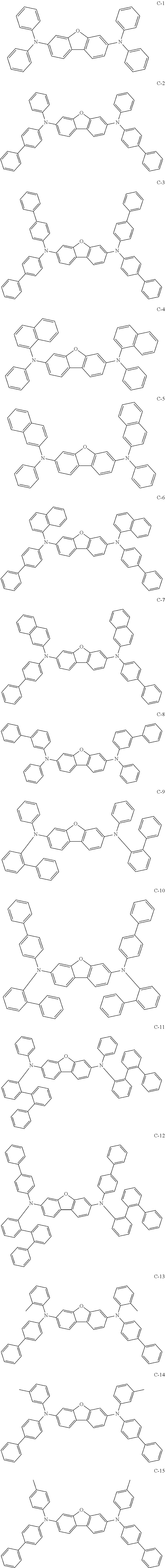 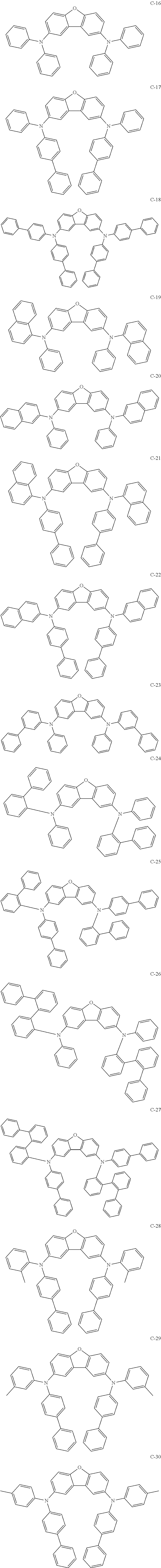 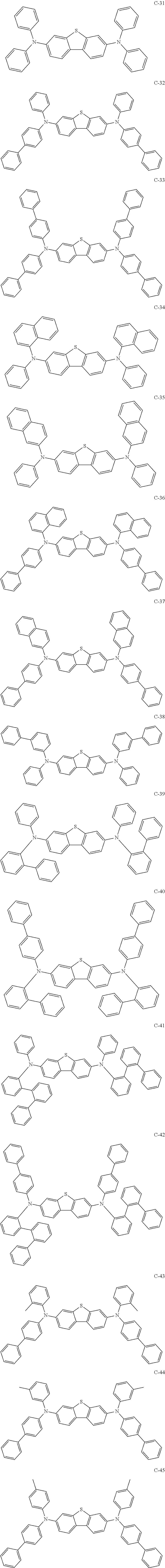 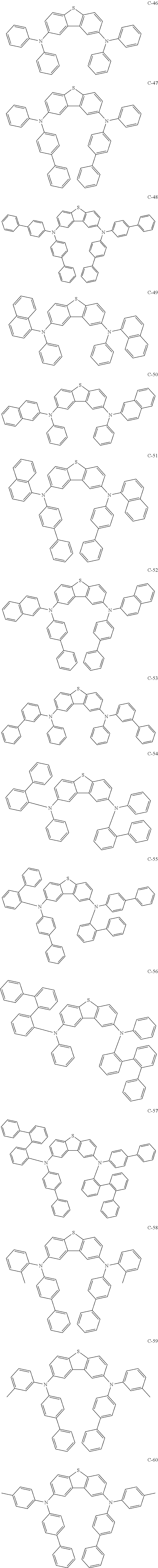 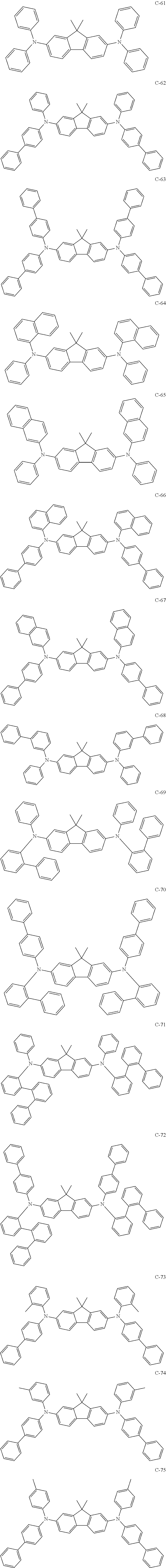 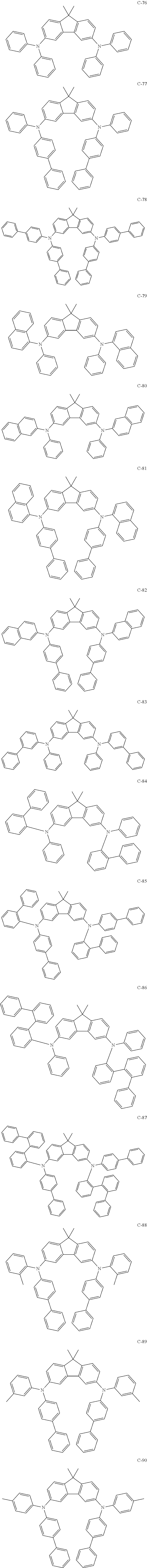 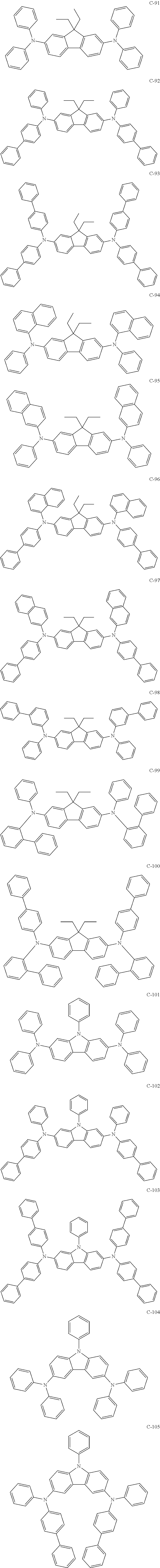 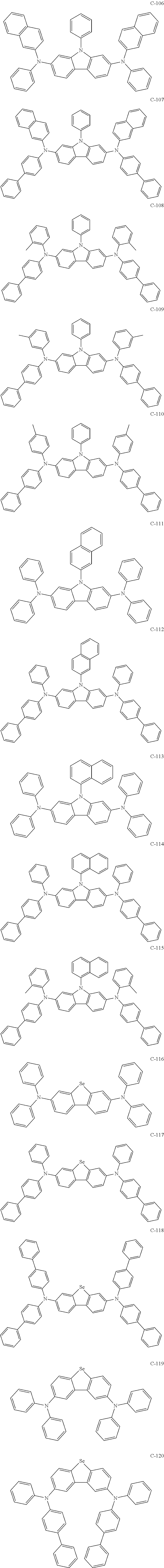 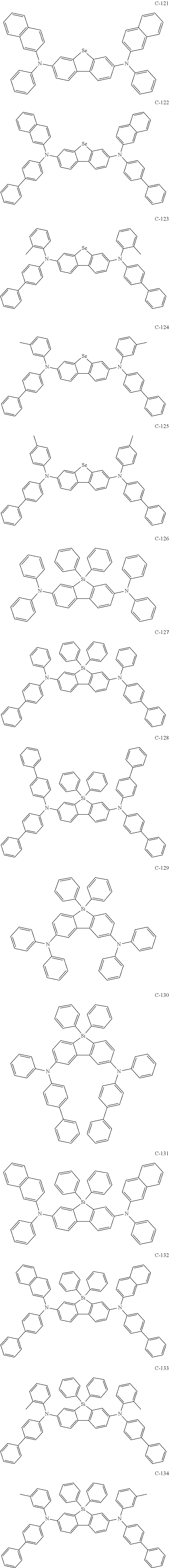 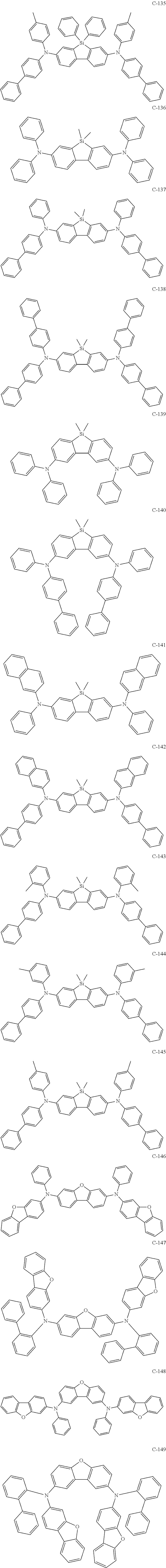 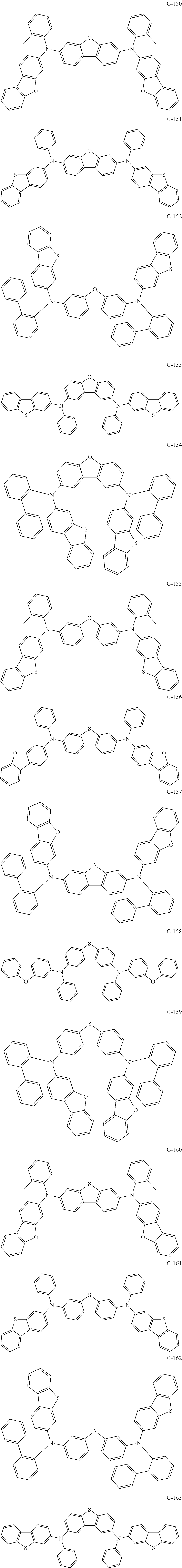 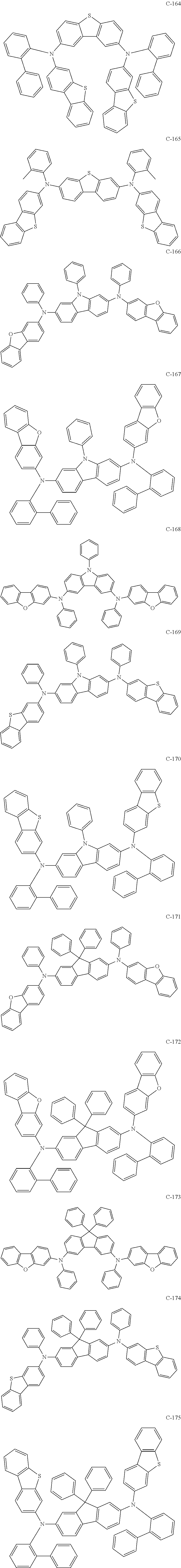 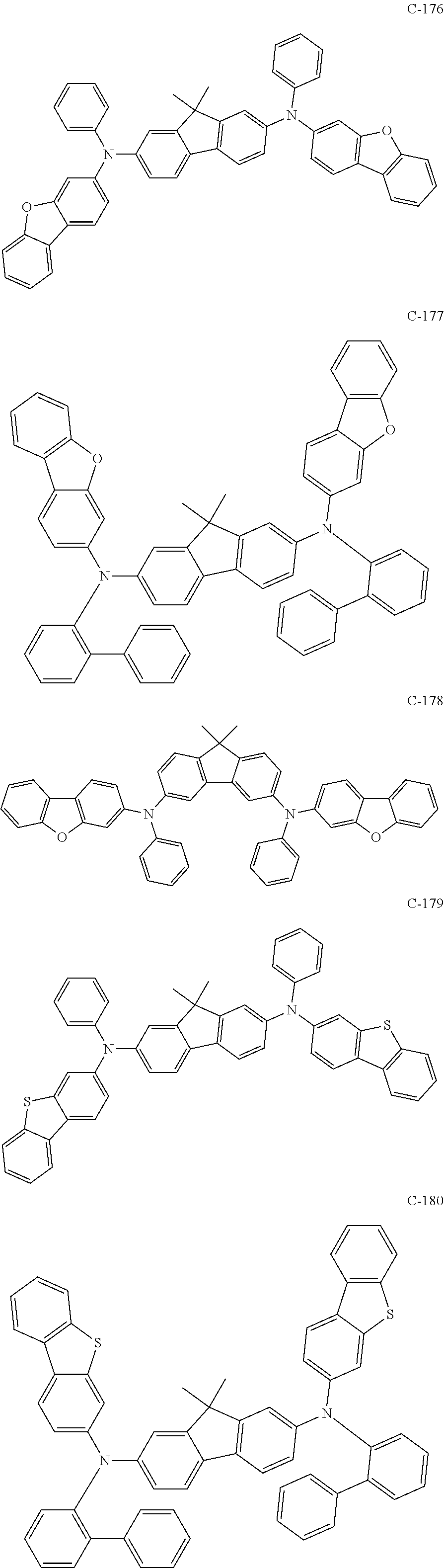 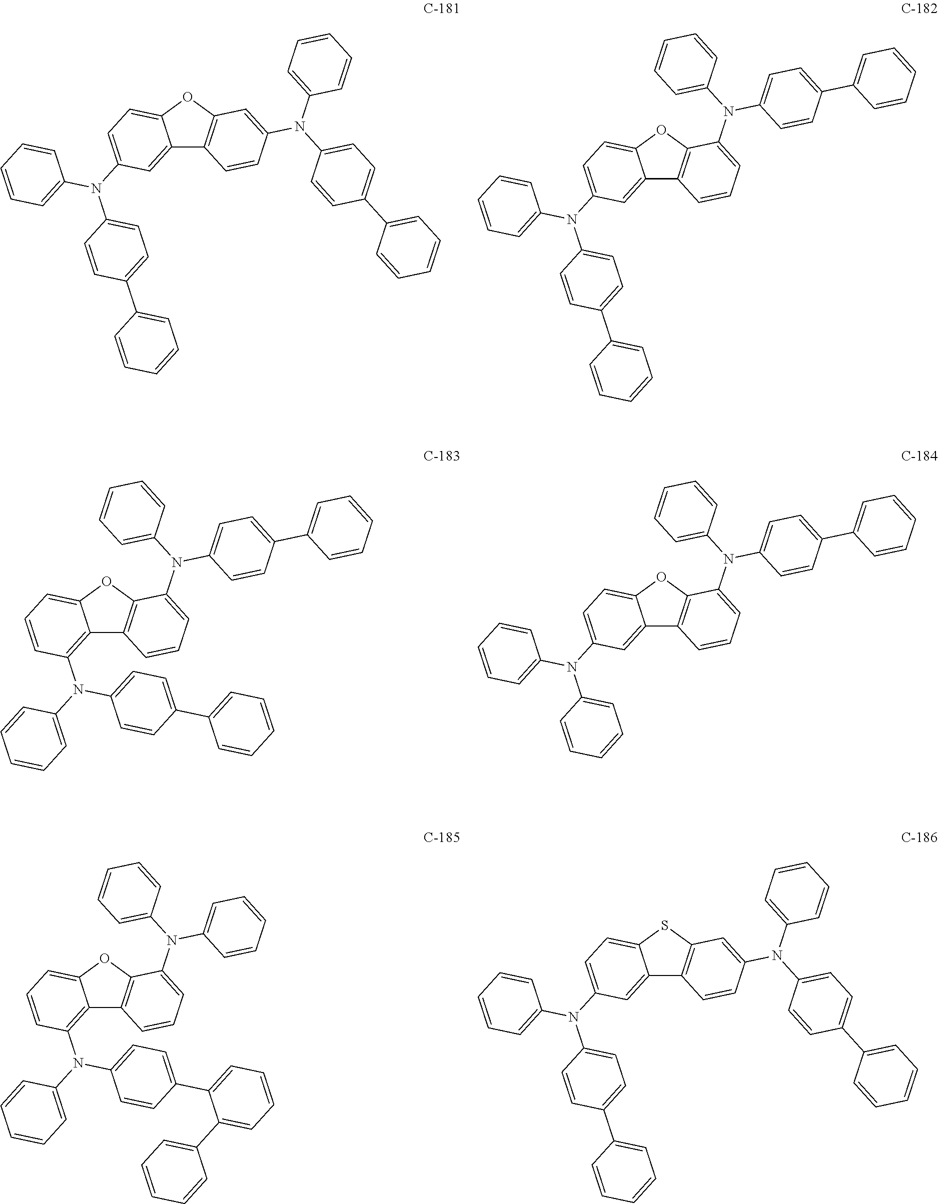 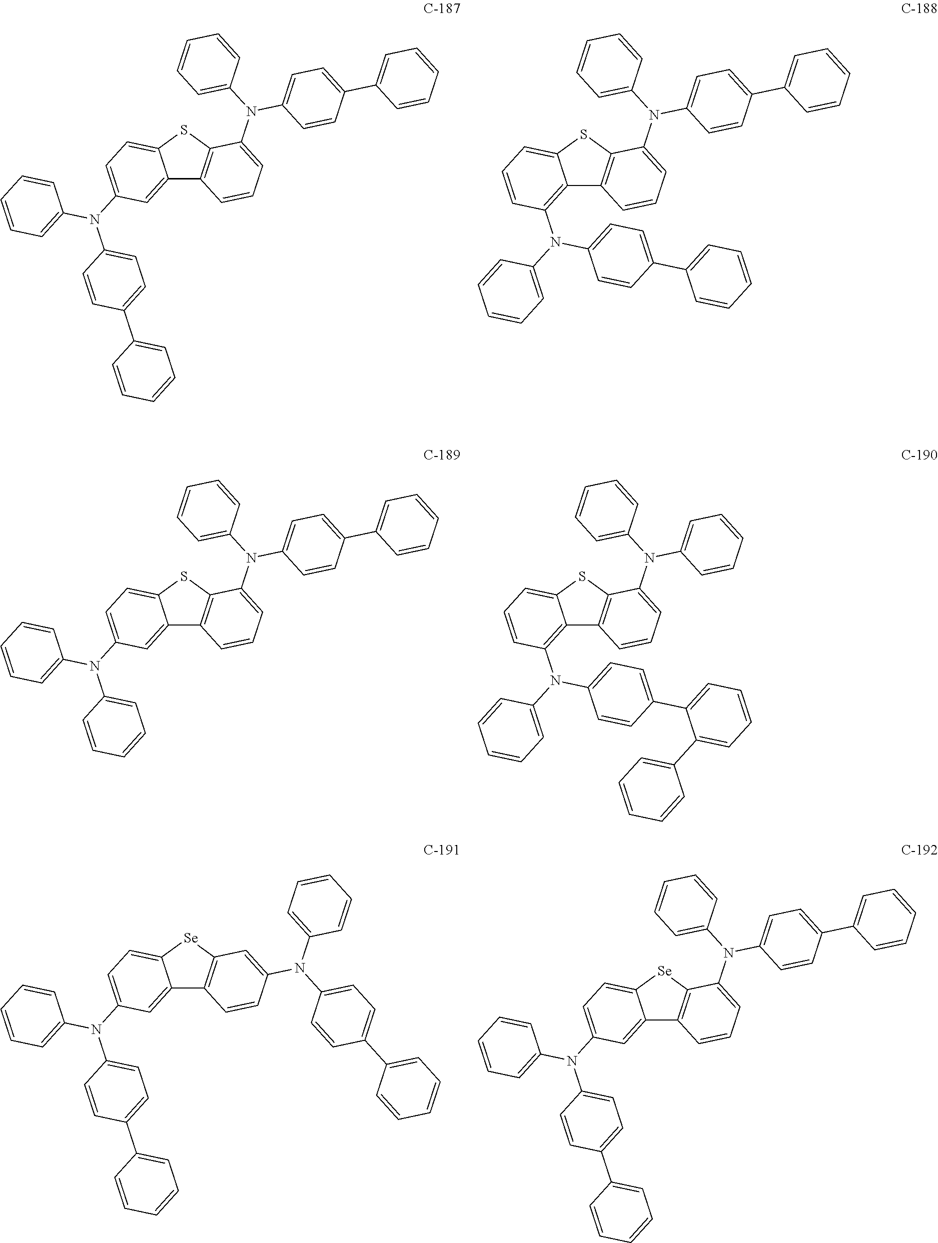 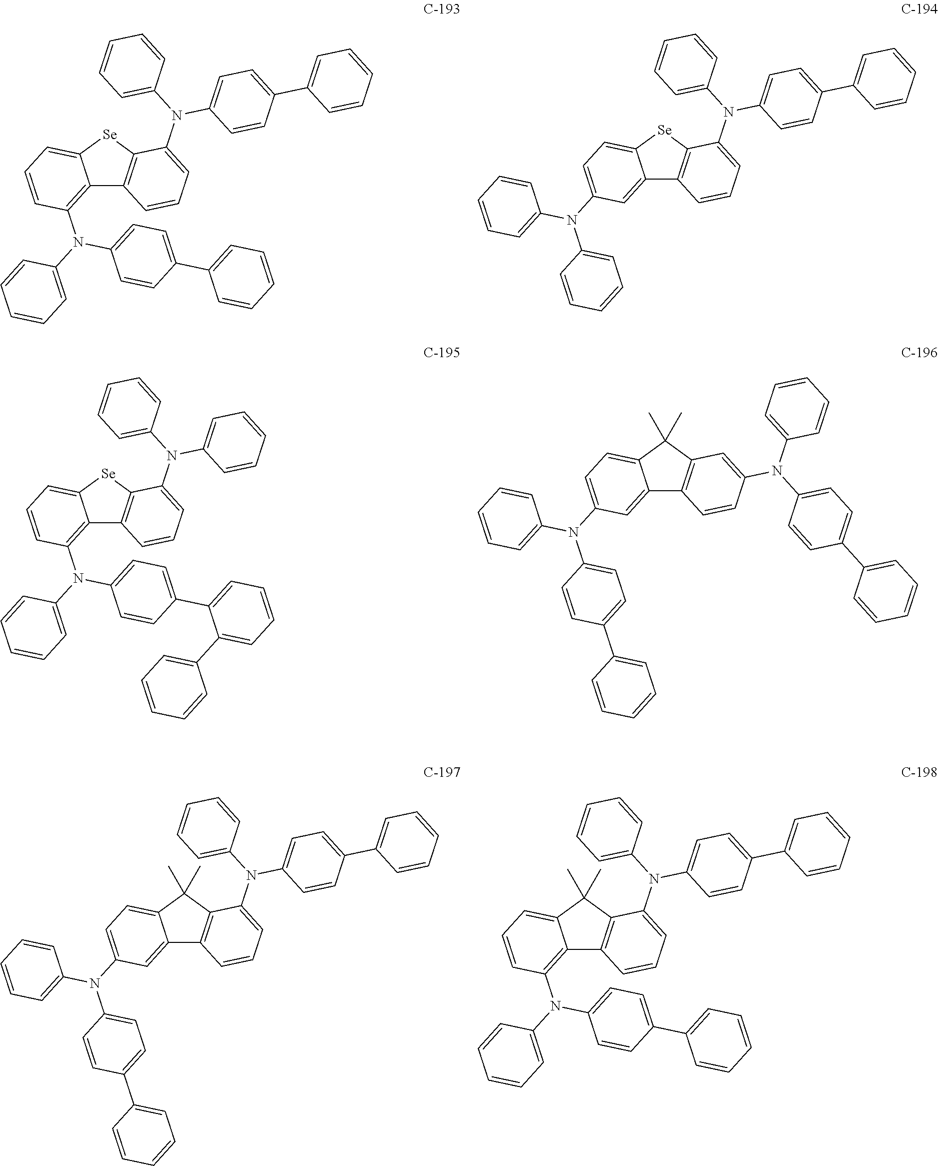 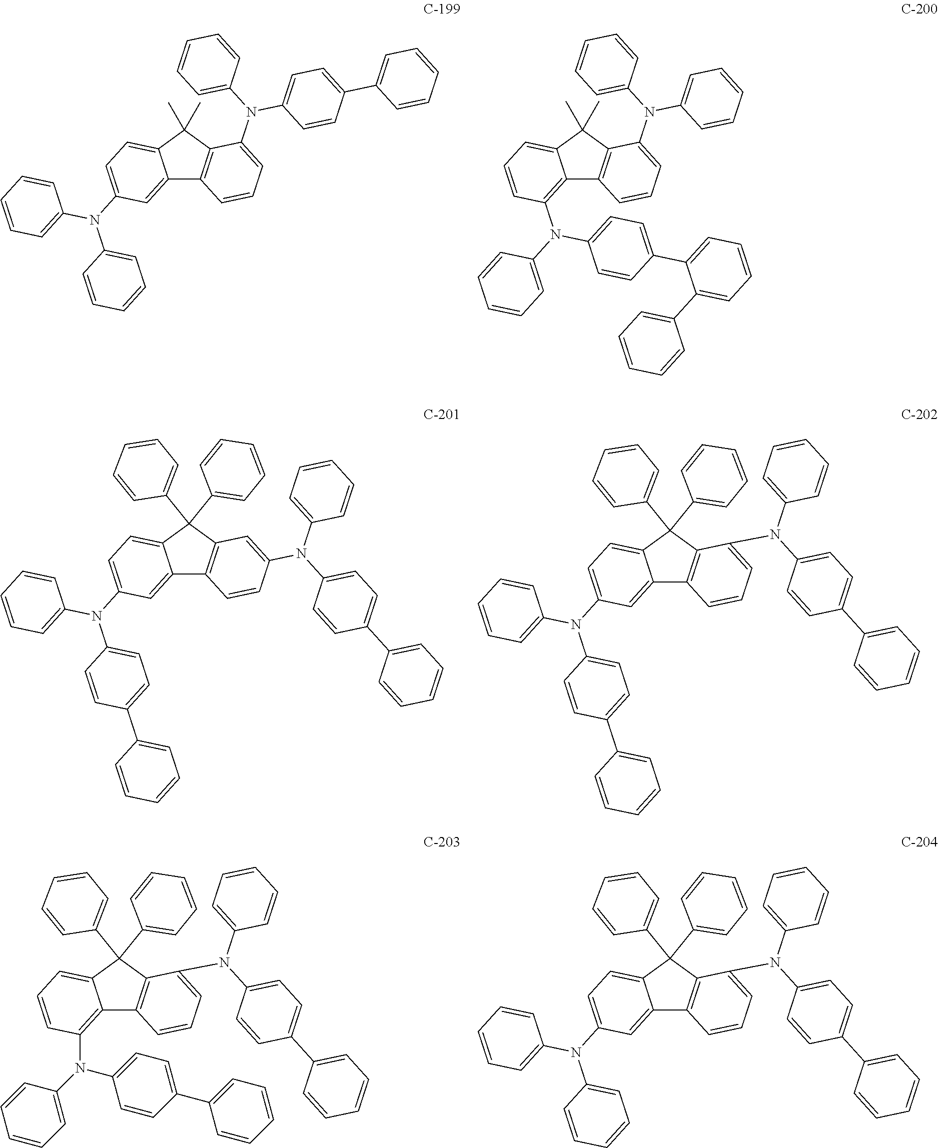 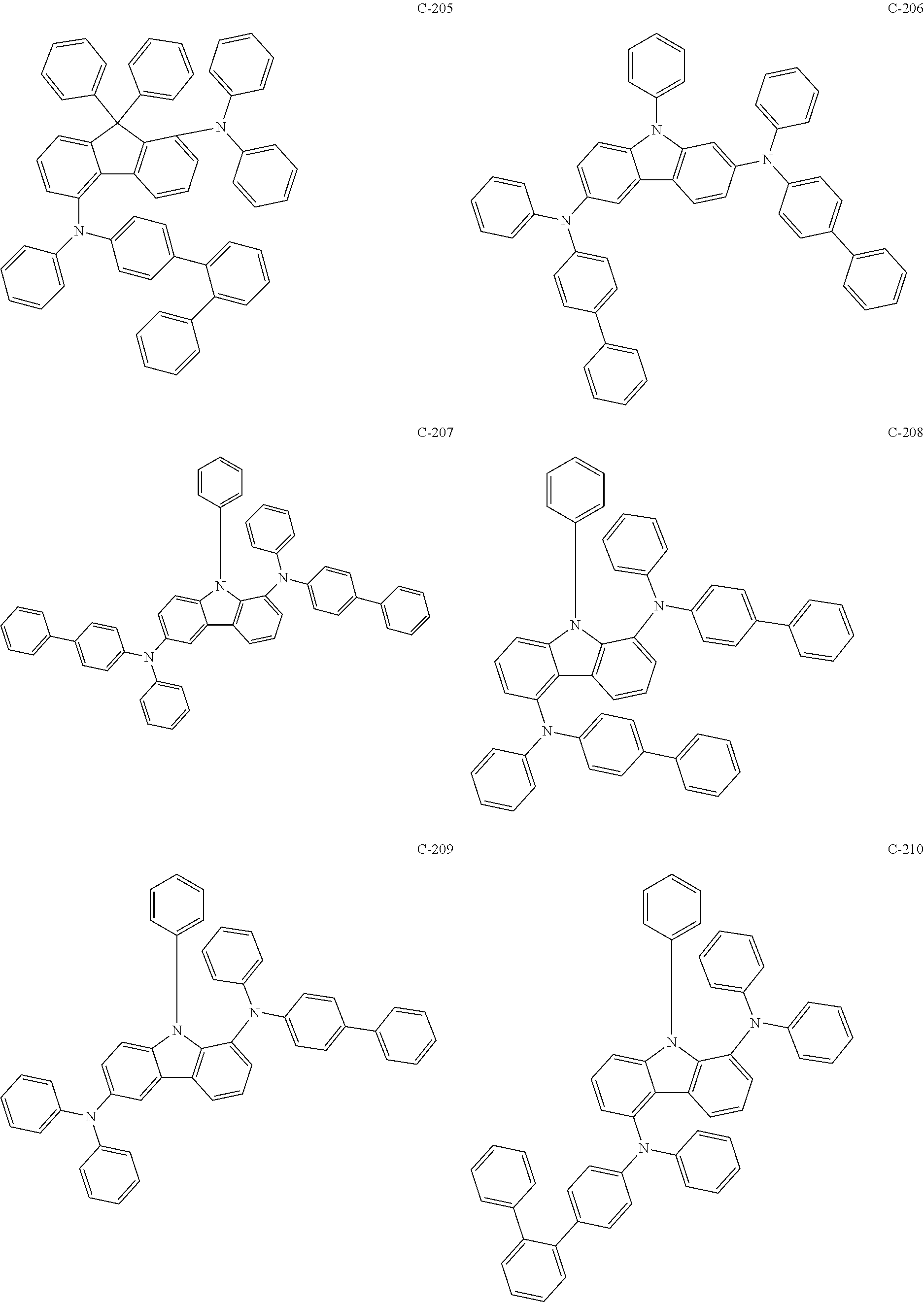 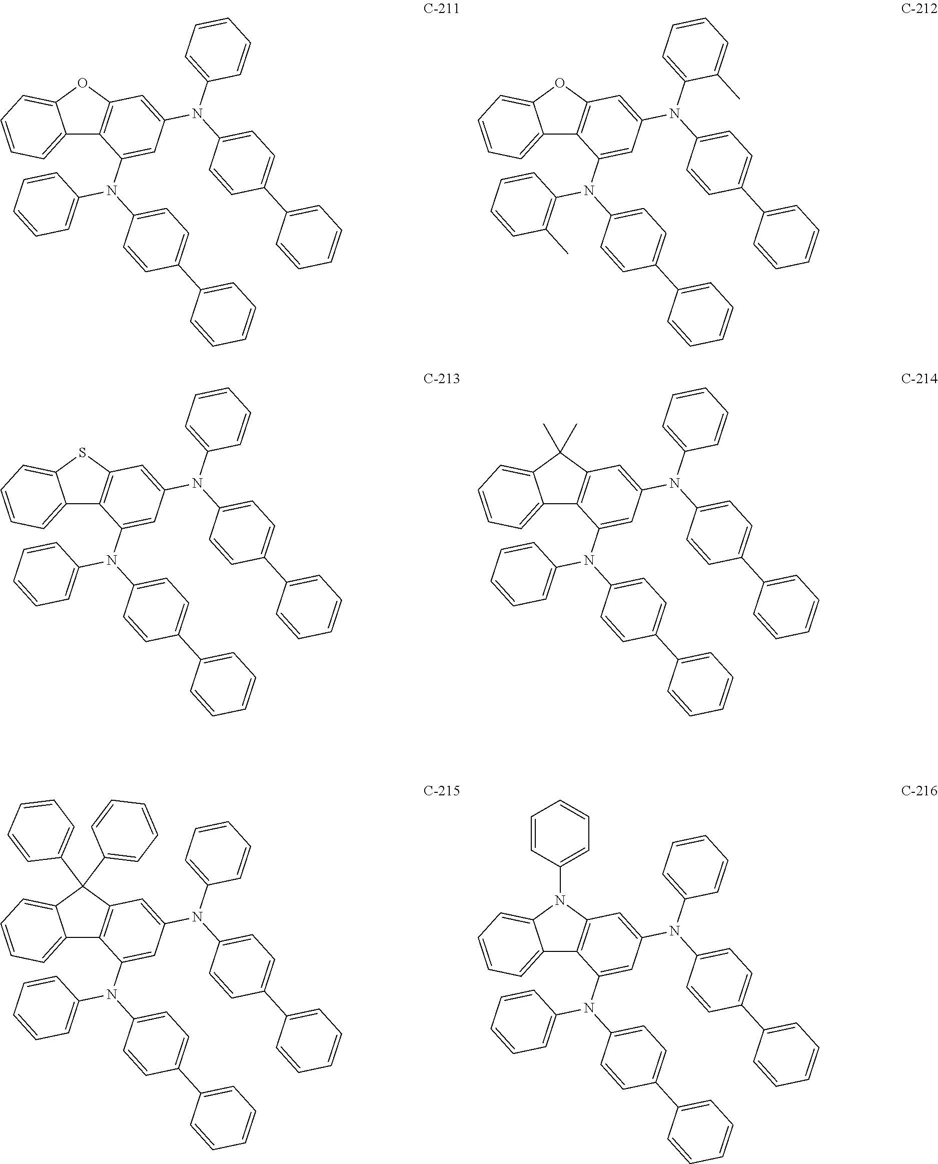 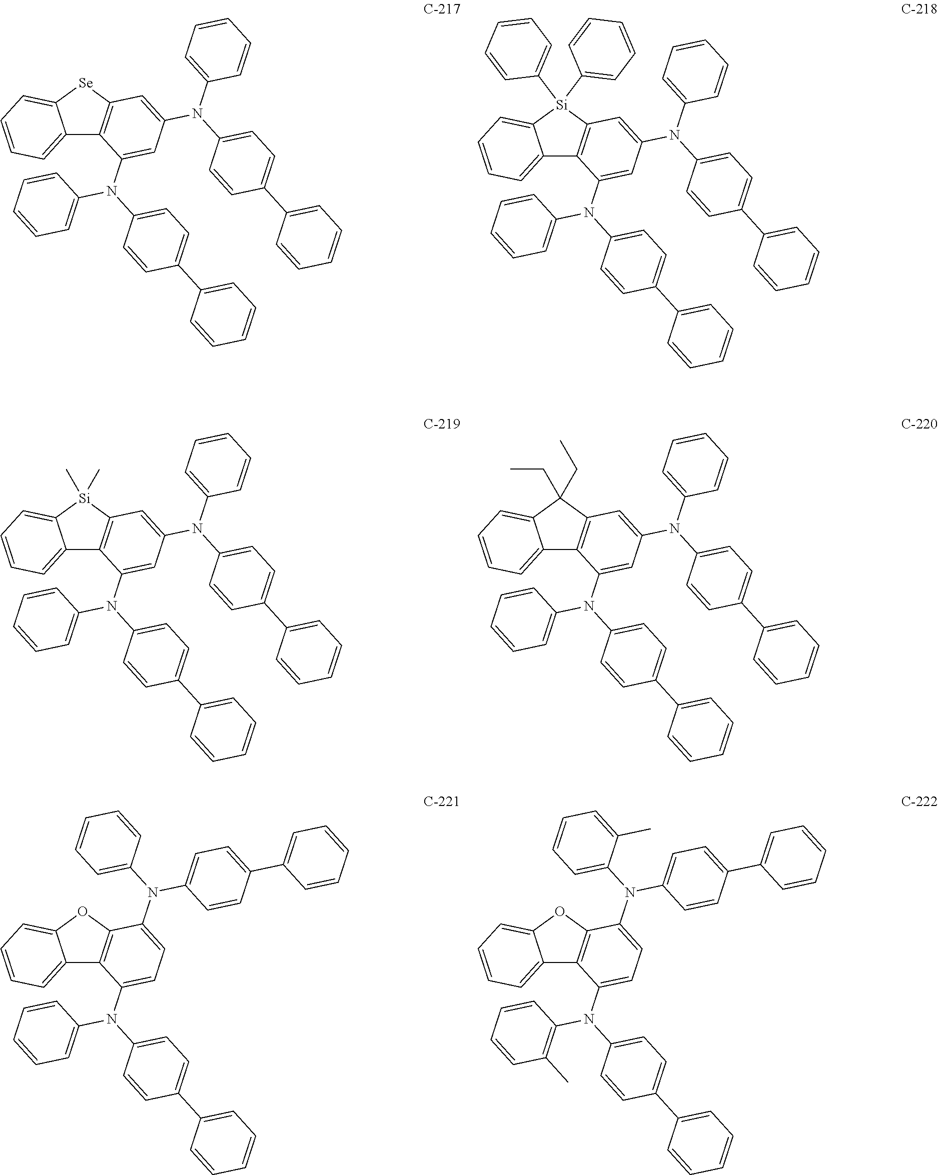 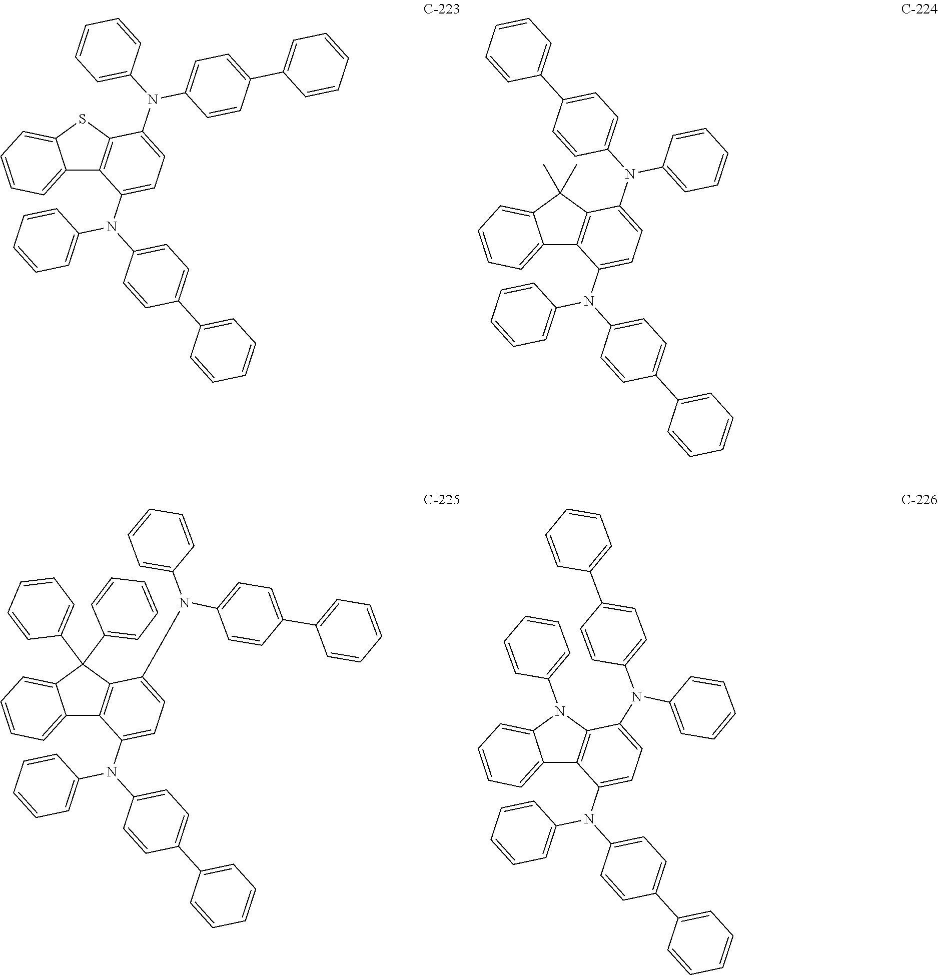 [SMILES: c1ccc(-c2ccc(N(c3ccccc3)c3ccc(N(c4ccccc4)c4ccc(-c5ccccc5)cc4)c4c3sc3ccccc34)cc2)cc1 ; CC1(C)c2ccccc2-c2c(N(c3ccccc3)c3ccc(-c4ccccc4)cc3)ccc(N(c3ccccc3)c3ccc(-c4ccccc4)cc3)c21 ; c1ccc(-c2ccc(N(c3ccccc3)c3ccc(N(c4ccccc4)c4ccc(-c5ccccc5)cc4)c4c3-c3ccccc3C4(c3ccccc3)c3ccccc3)cc2)cc1 ; c1ccc(-c2ccc(N(c3ccccc3)c3ccc(N(c4ccccc4)c4ccc(-c5ccccc5)cc4)c4c3c3ccccc3n4-c3ccccc3)cc2)cc1] 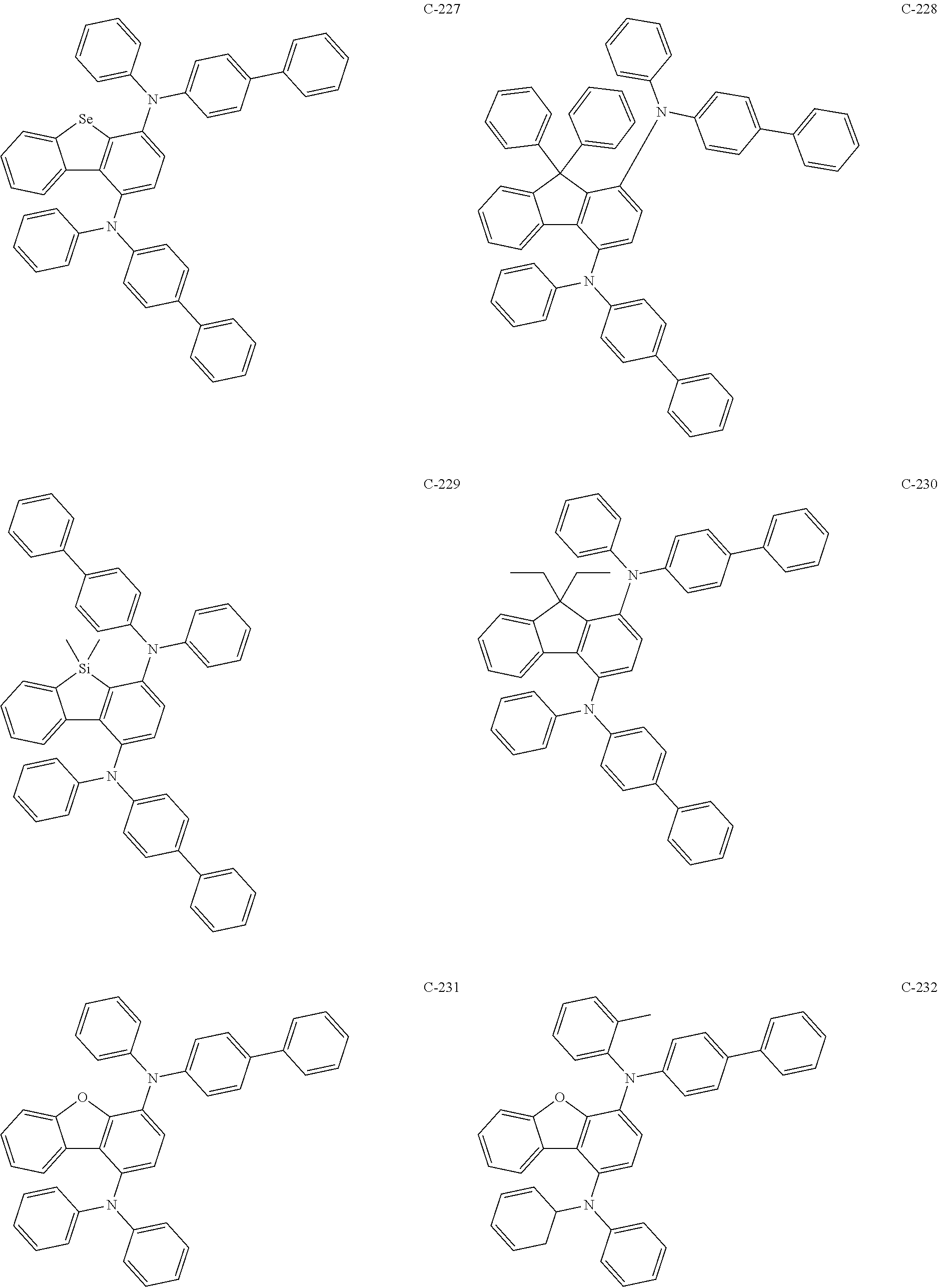 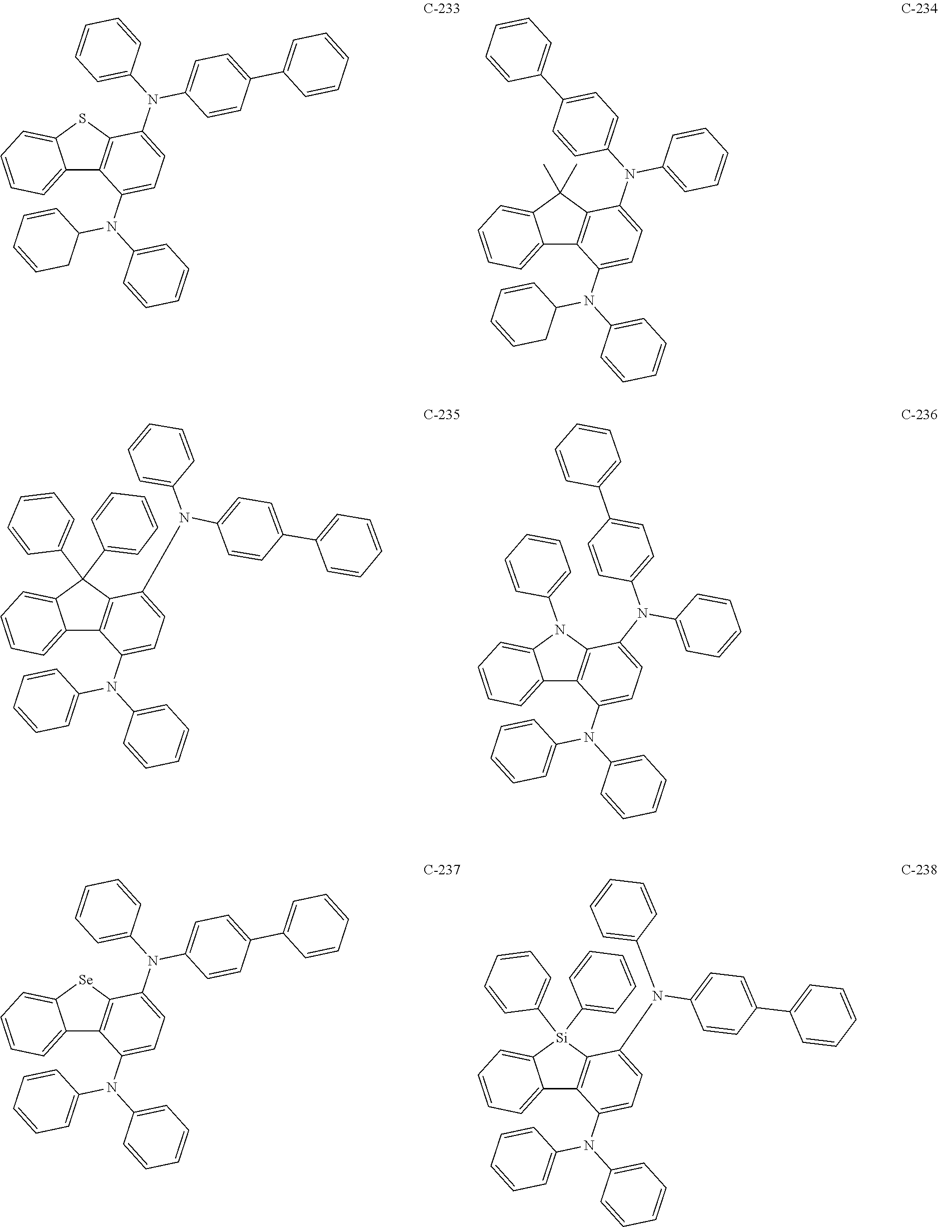 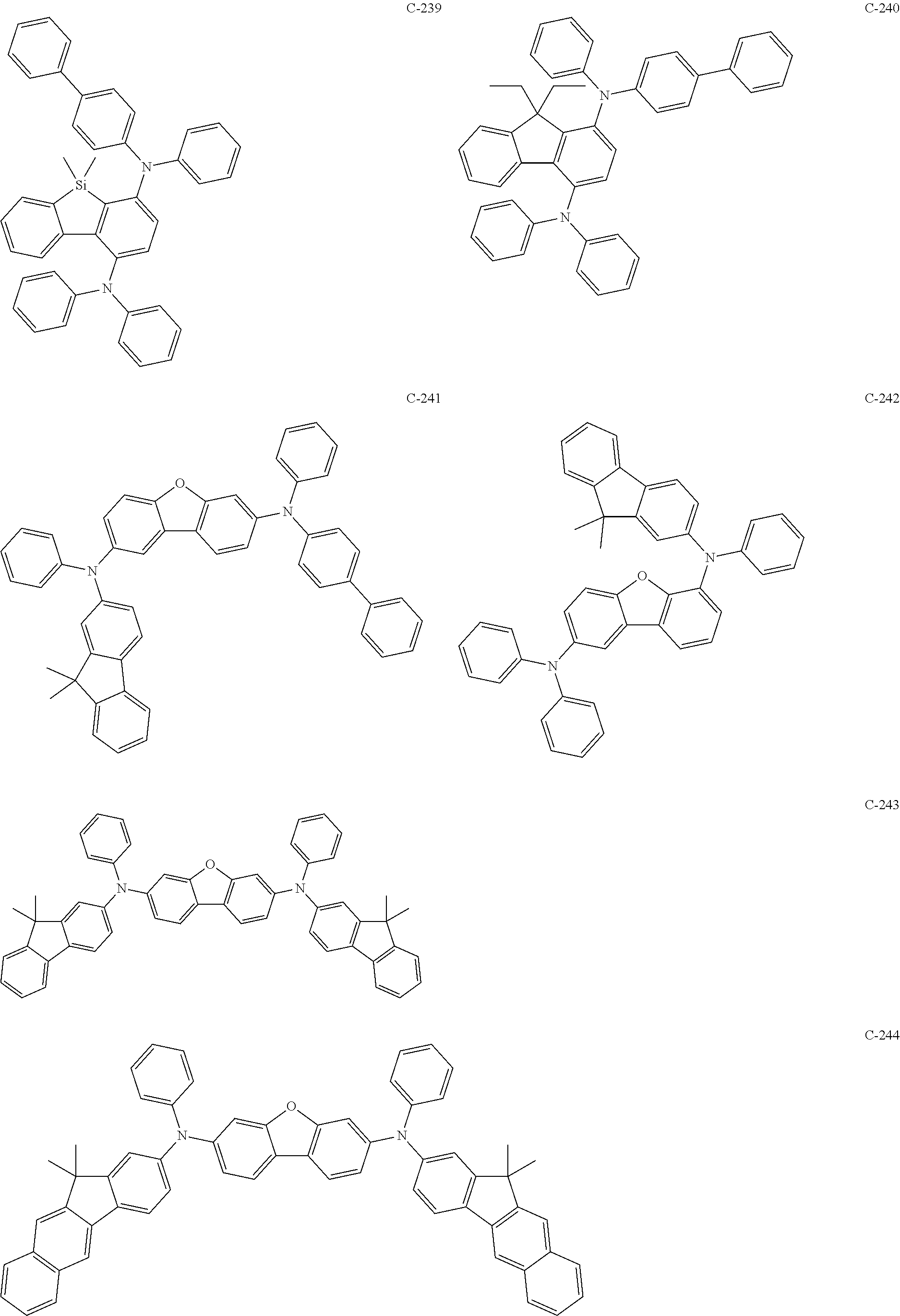 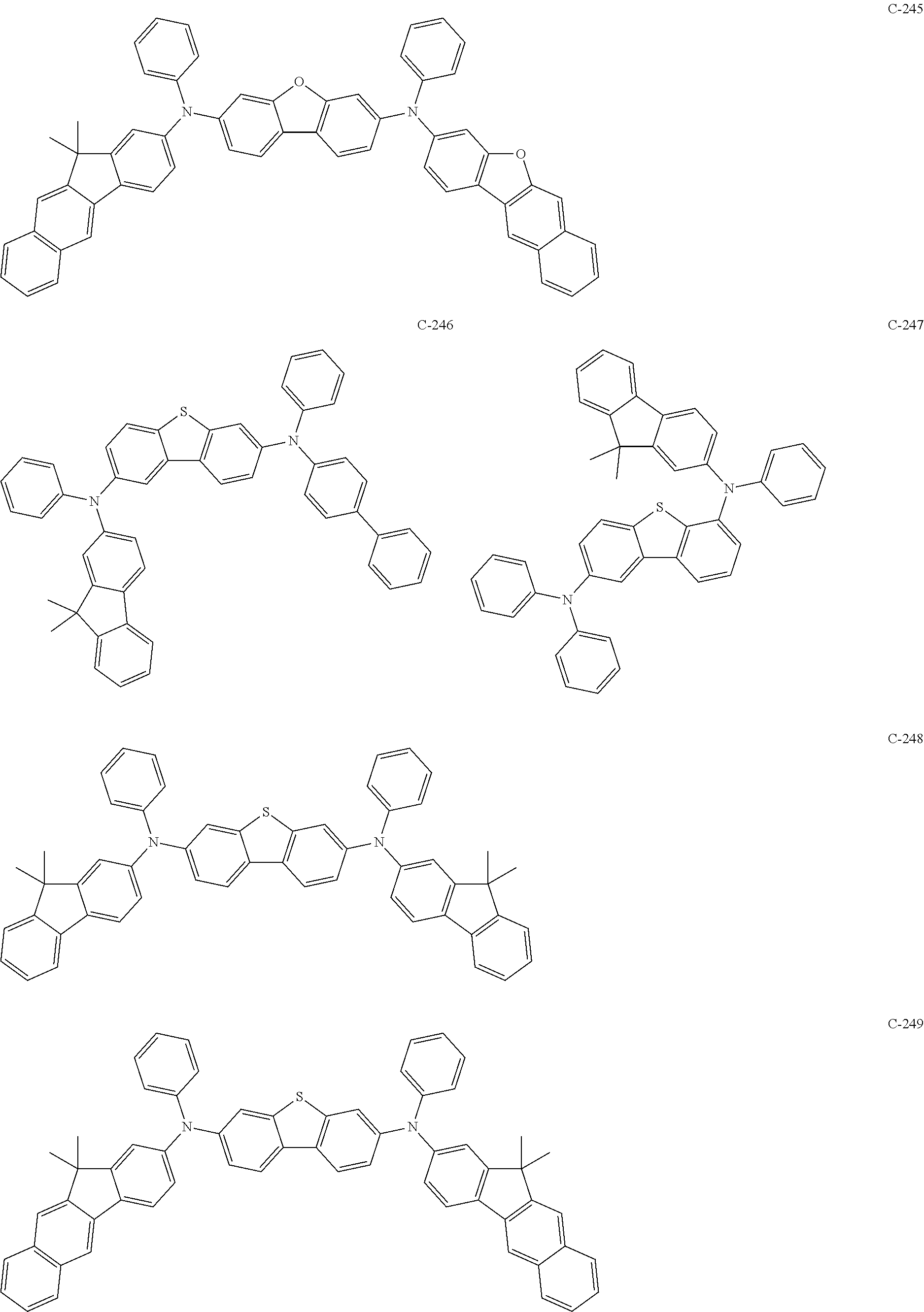 [SMILES: CC1(C)c2cc(N(c3ccccc3)c3ccc4c(c3)oc3cc(N(c5ccccc5)c5ccc6c(c5)oc5cc7ccccc7cc56)ccc34)ccc2-c2cc3ccccc3cc21 ; CC1(C)c2ccccc2-c2ccc(N(c3ccccc3)c3ccc4sc5cc(N(c6ccccc6)c6ccc(-c7ccccc7)cc6)ccc5c4c3)cc21 ; CC1(C)c2ccccc2-c2ccc(N(c3ccccc3)c3cccc4c3sc3ccc(N(c5ccccc5)c5ccccc5)cc34)cc21 ; CC1(C)c2ccccc2-c2ccc(N(c3ccccc3)c3ccc4c(c3)sc3cc(N(c5ccccc5)c5ccc6c(c5)C(C)(C)c5ccccc5-6)ccc34)cc21 ; CC1(C)c2cc(N(c3ccccc3)c3ccc4c(c3)sc3cc(N(c5ccccc5)c5ccc6c(c5)C(C)(C)c5cc7ccccc7cc5-6)ccc34)ccc2-c2cc3ccccc3cc21] 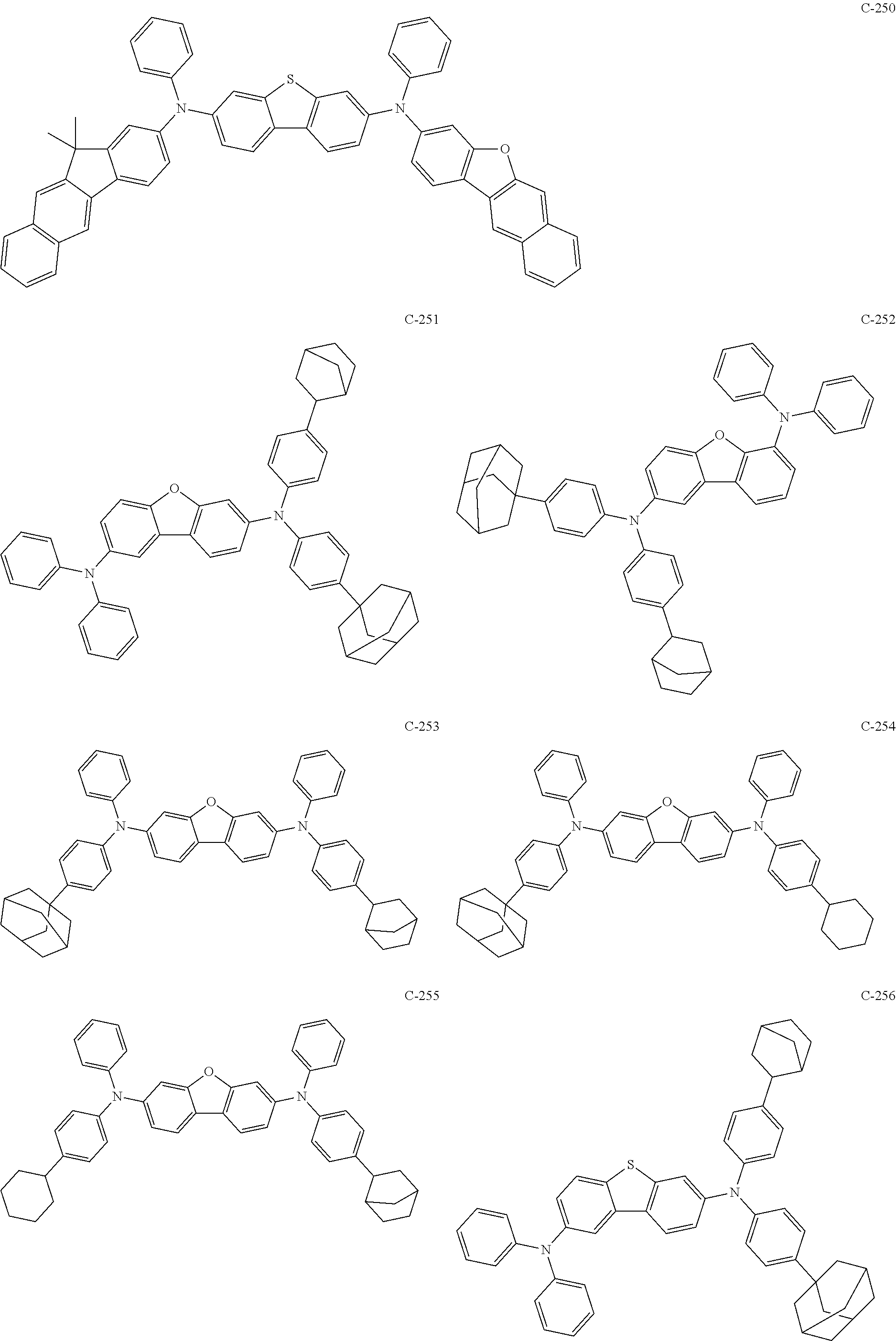 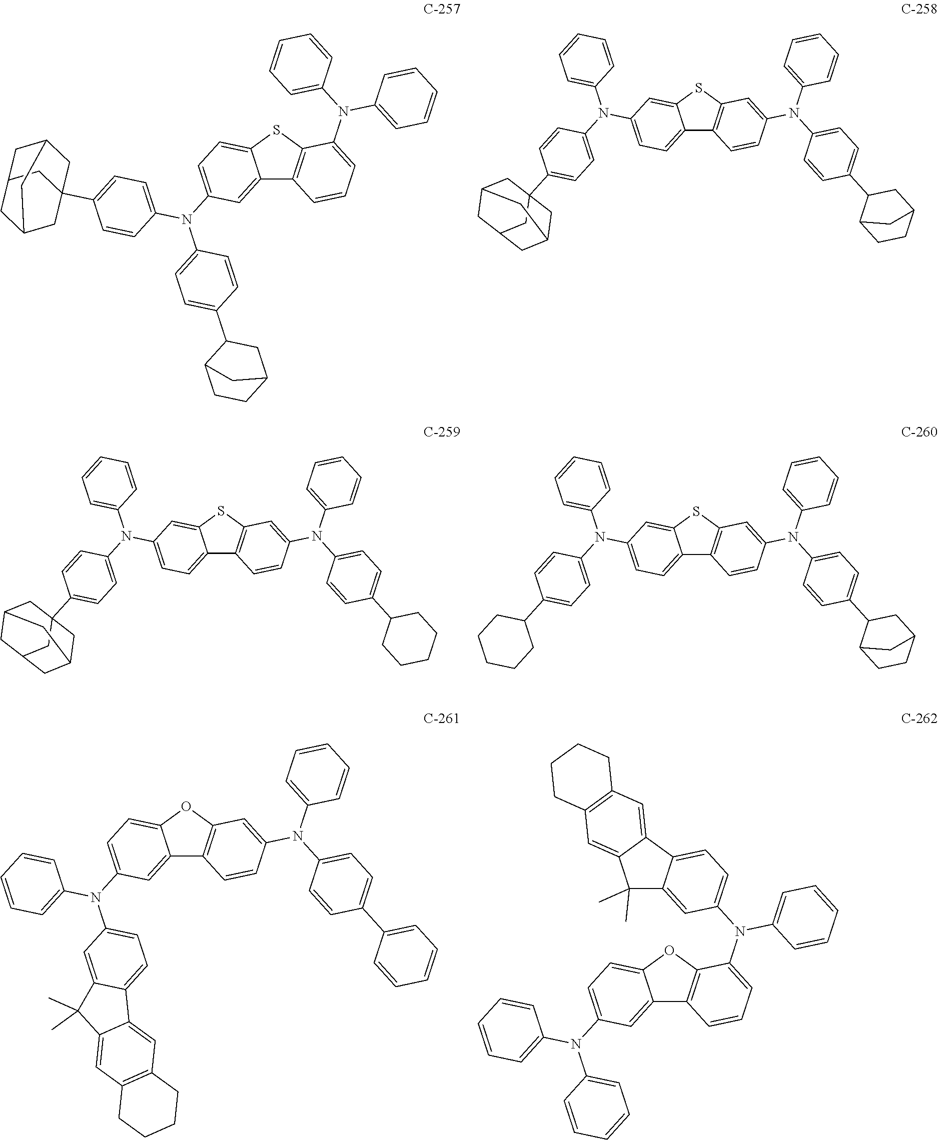 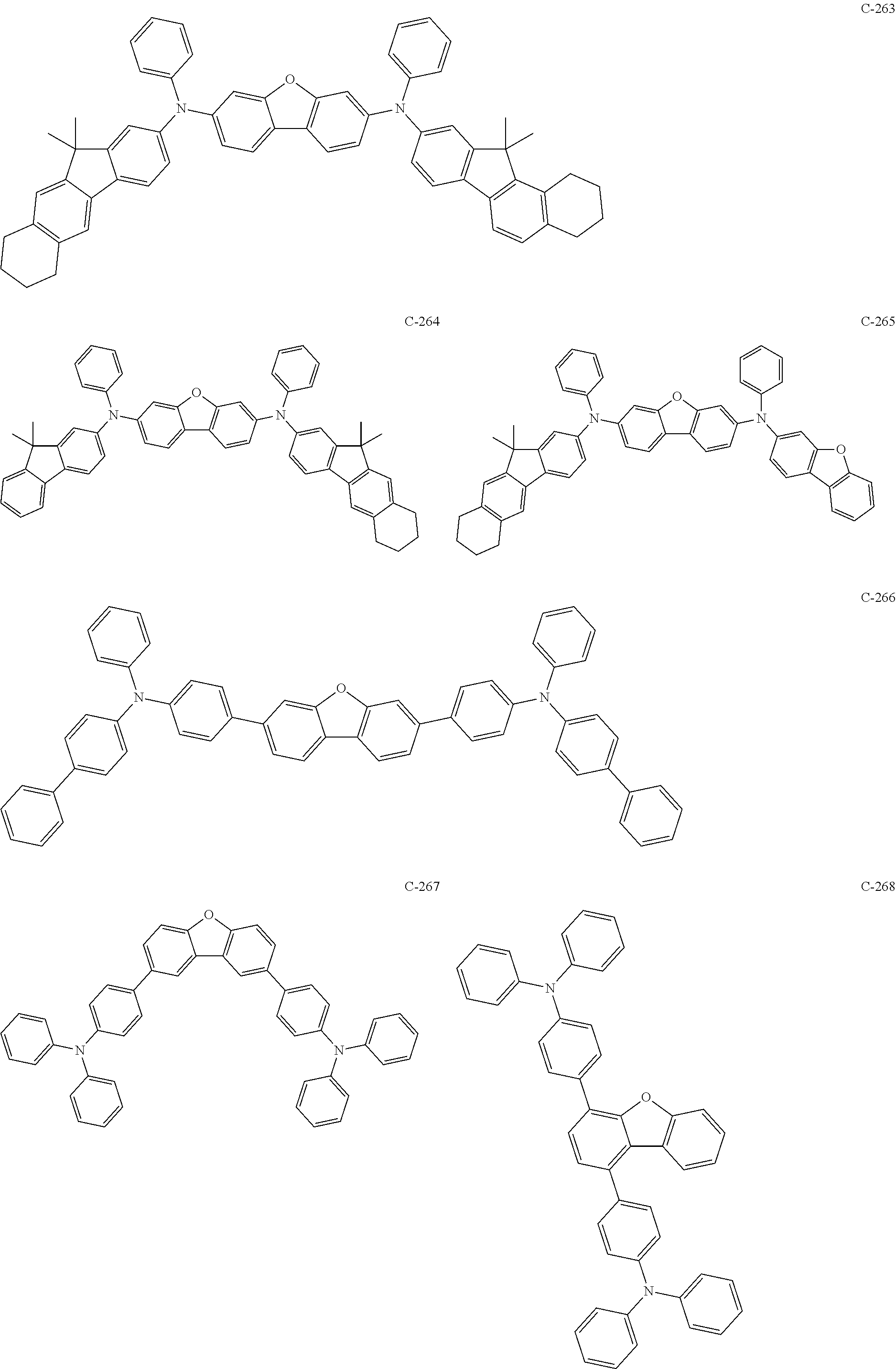 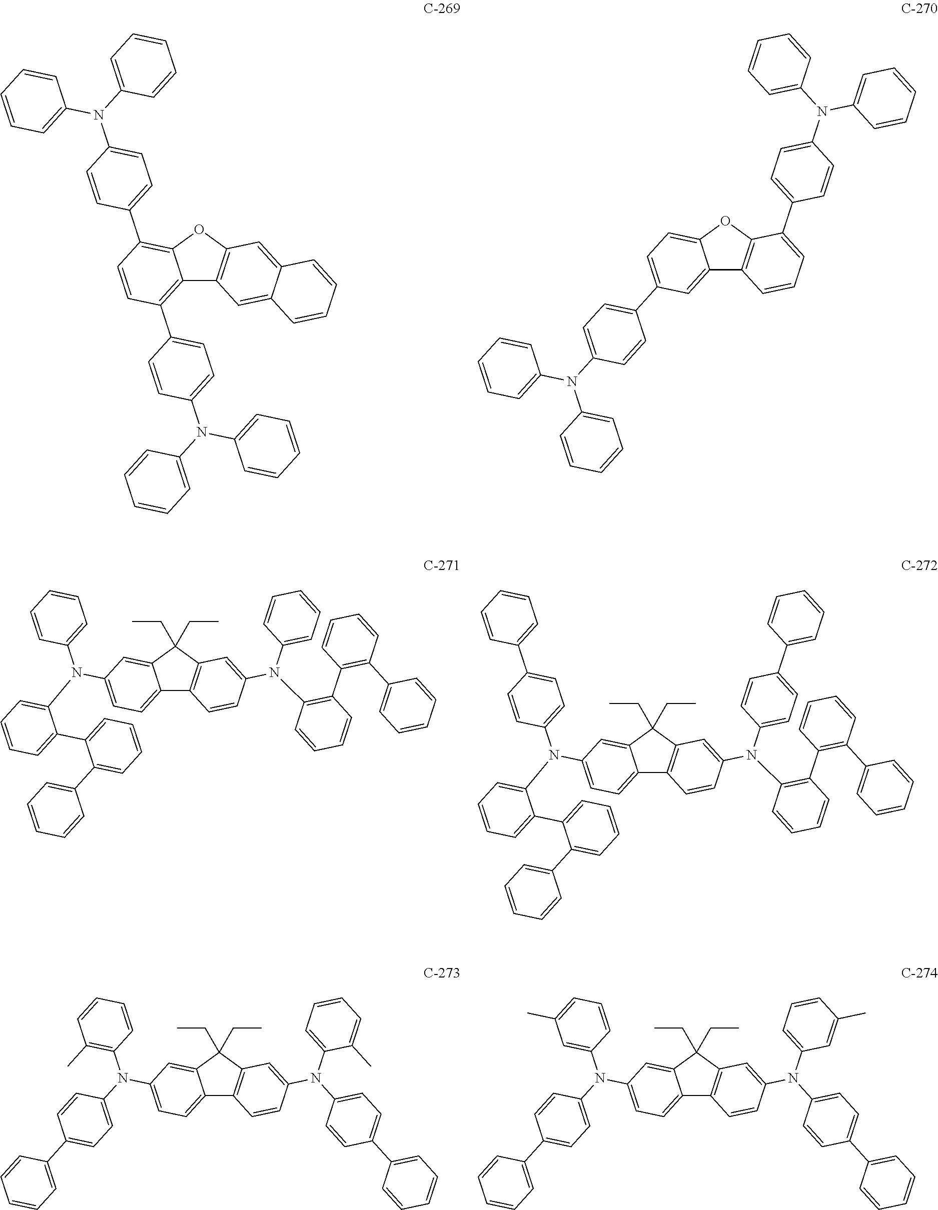 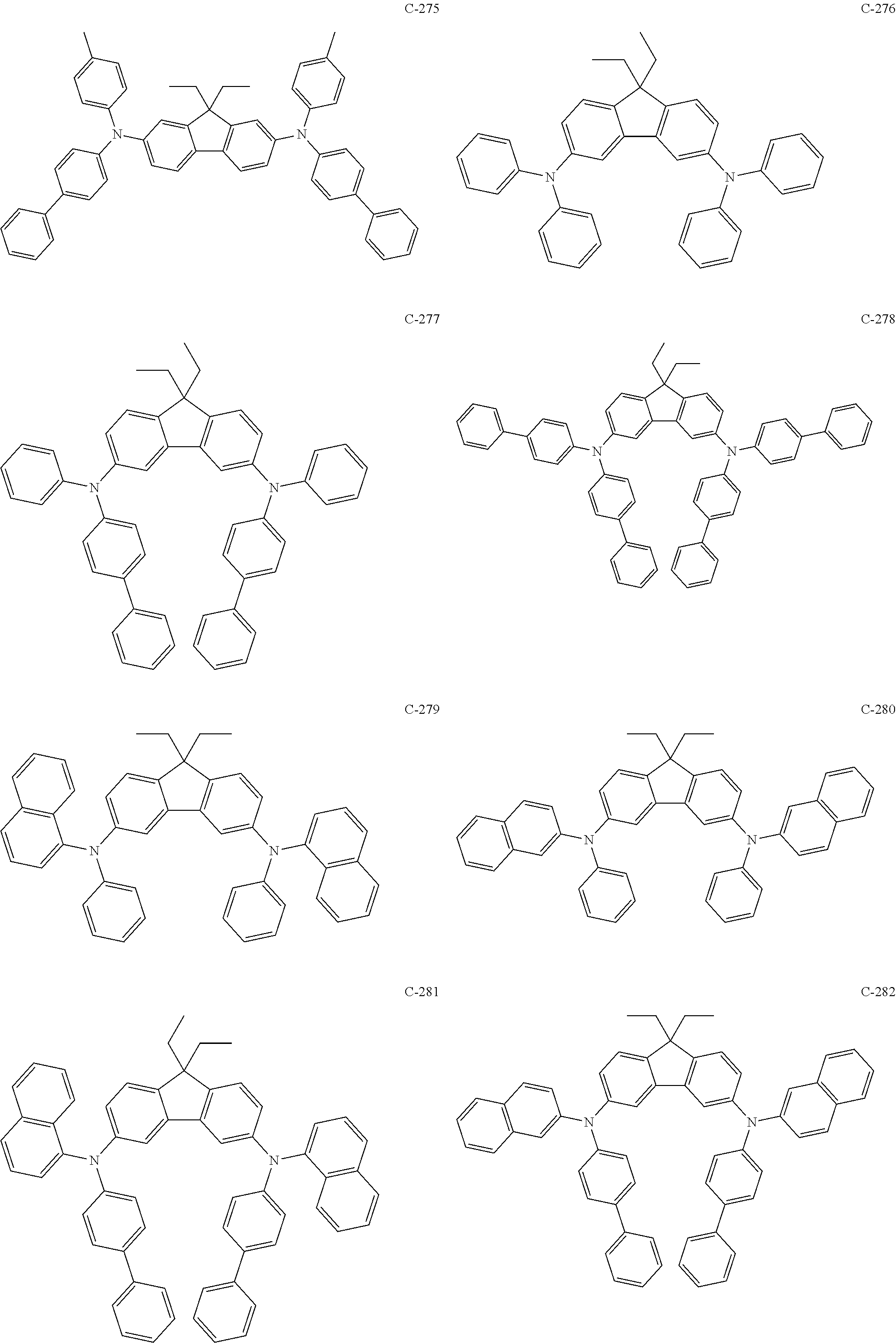 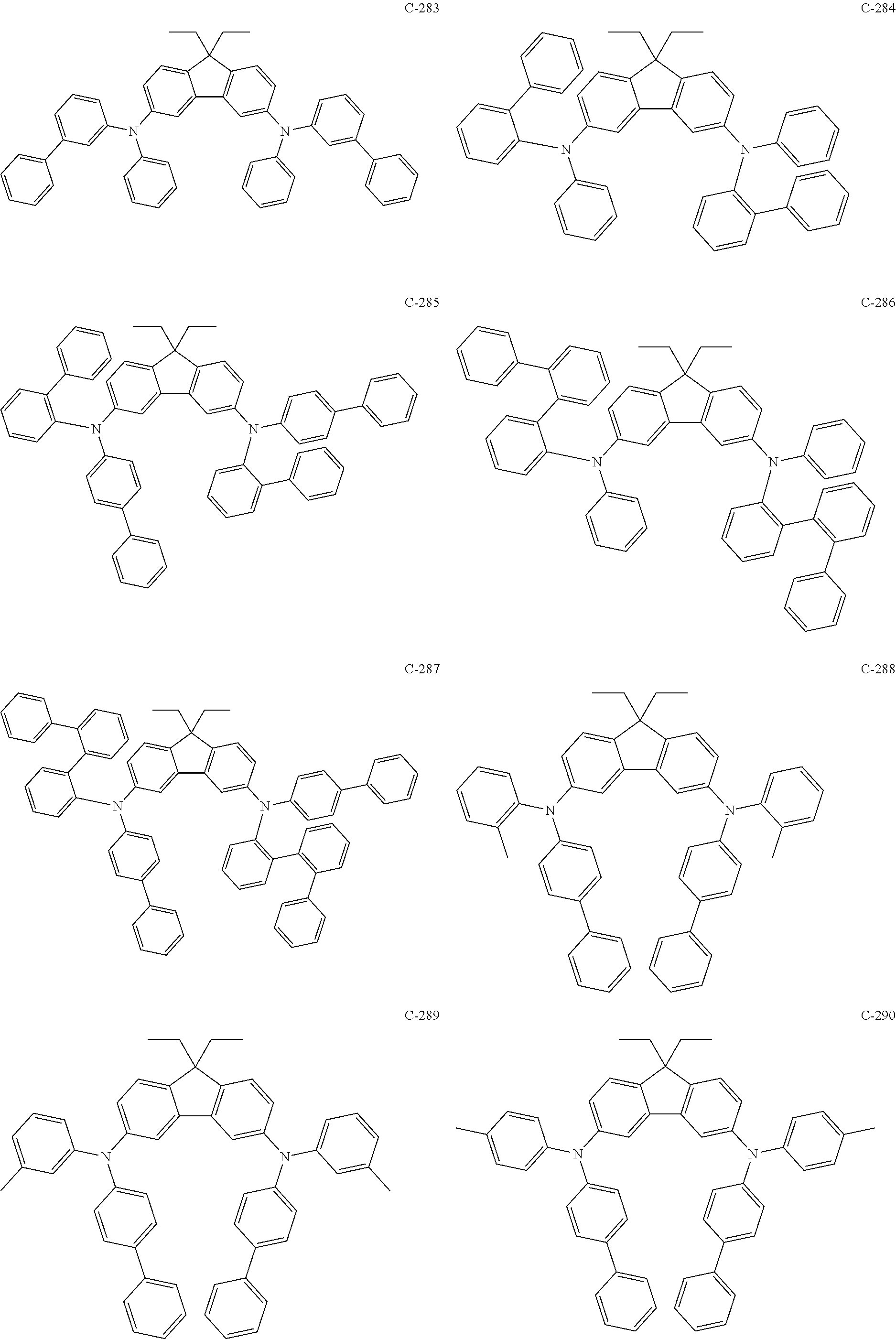 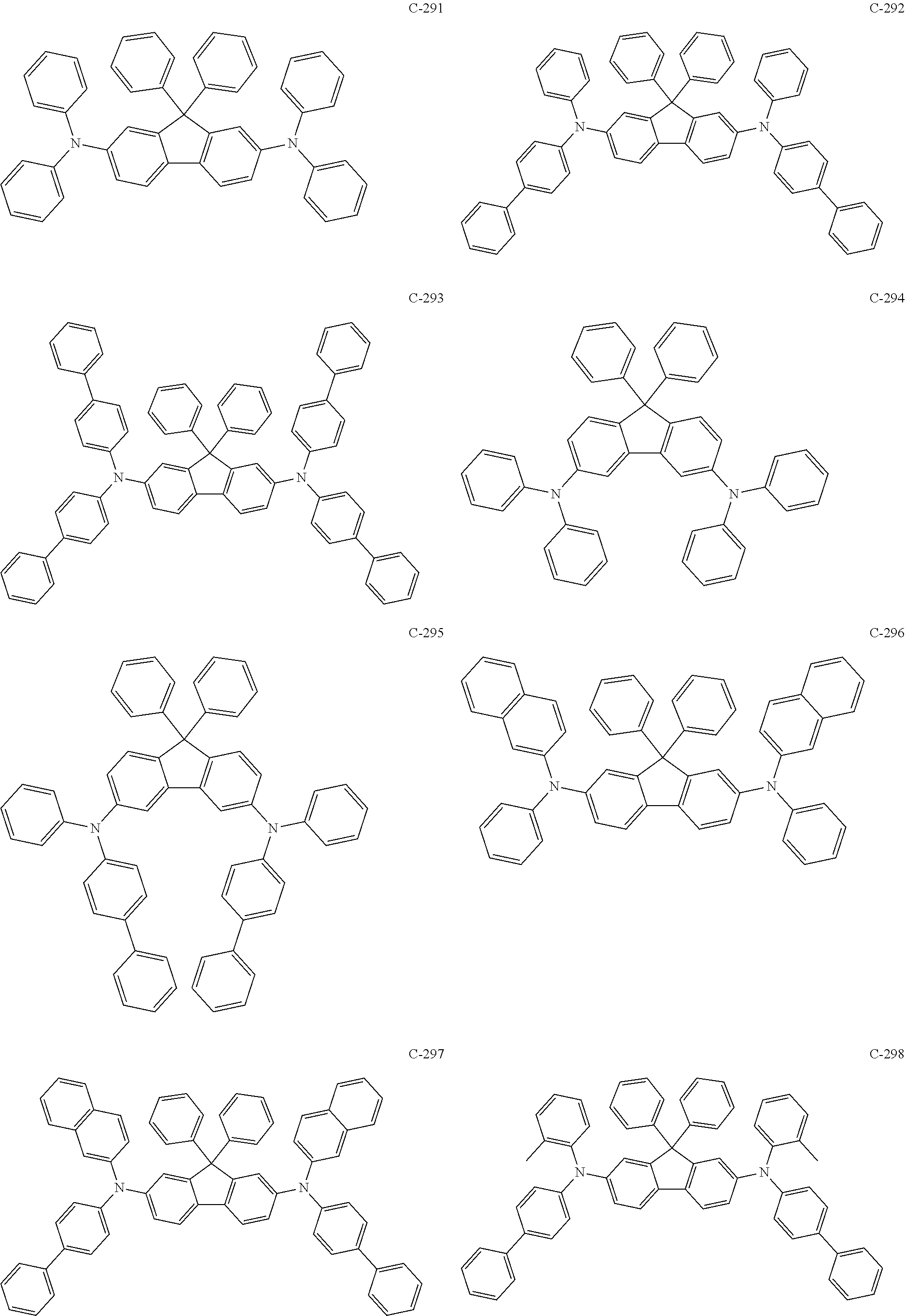 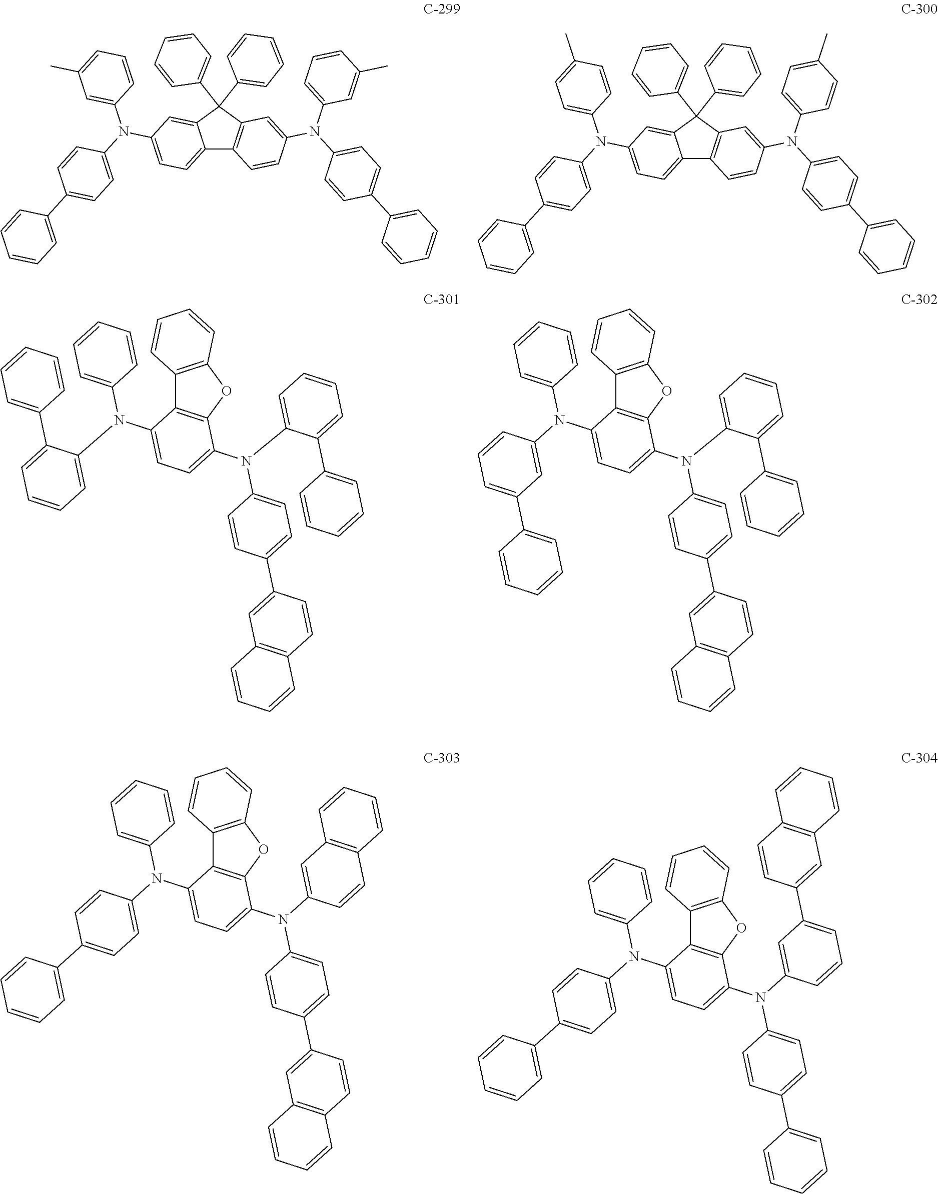 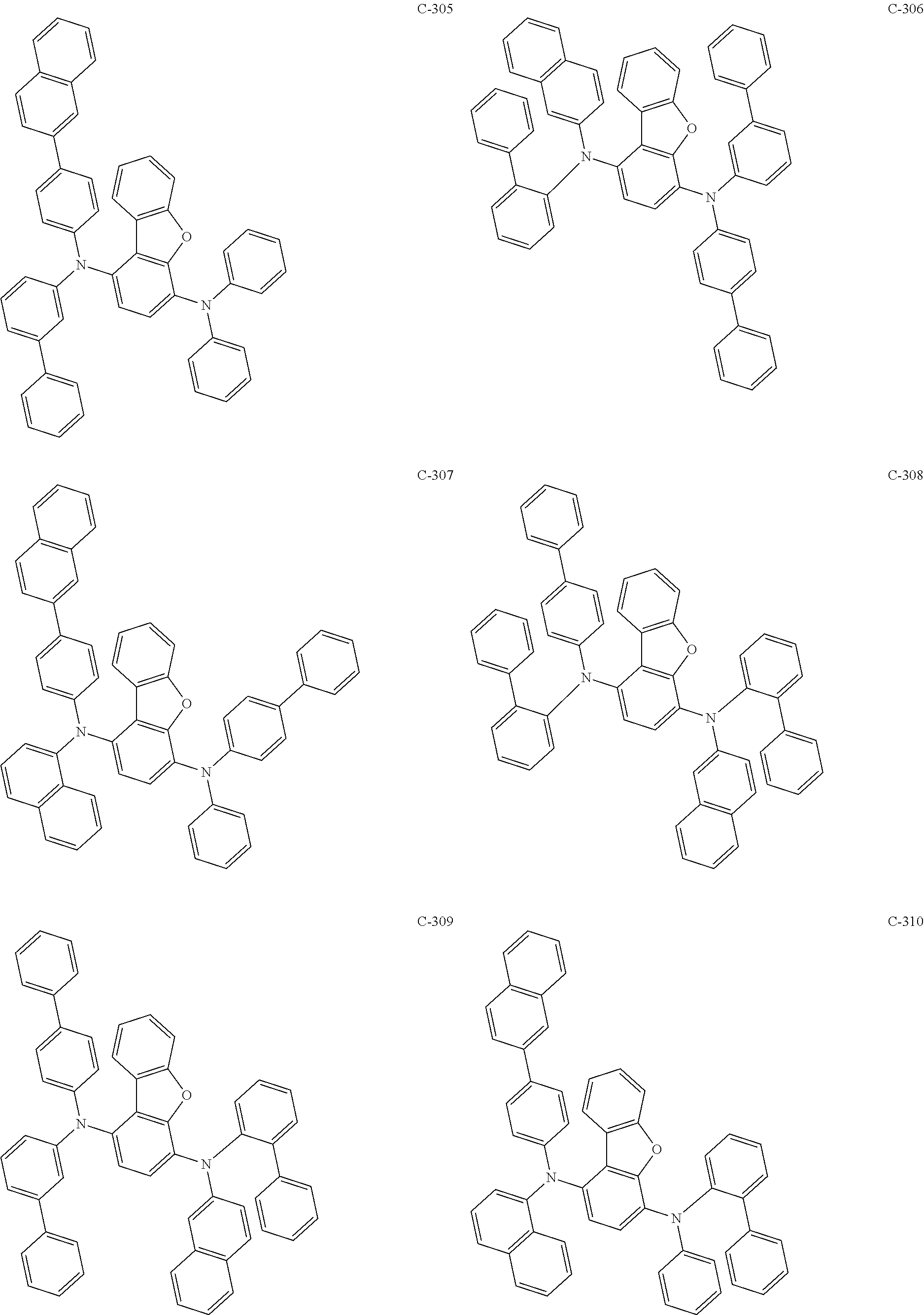 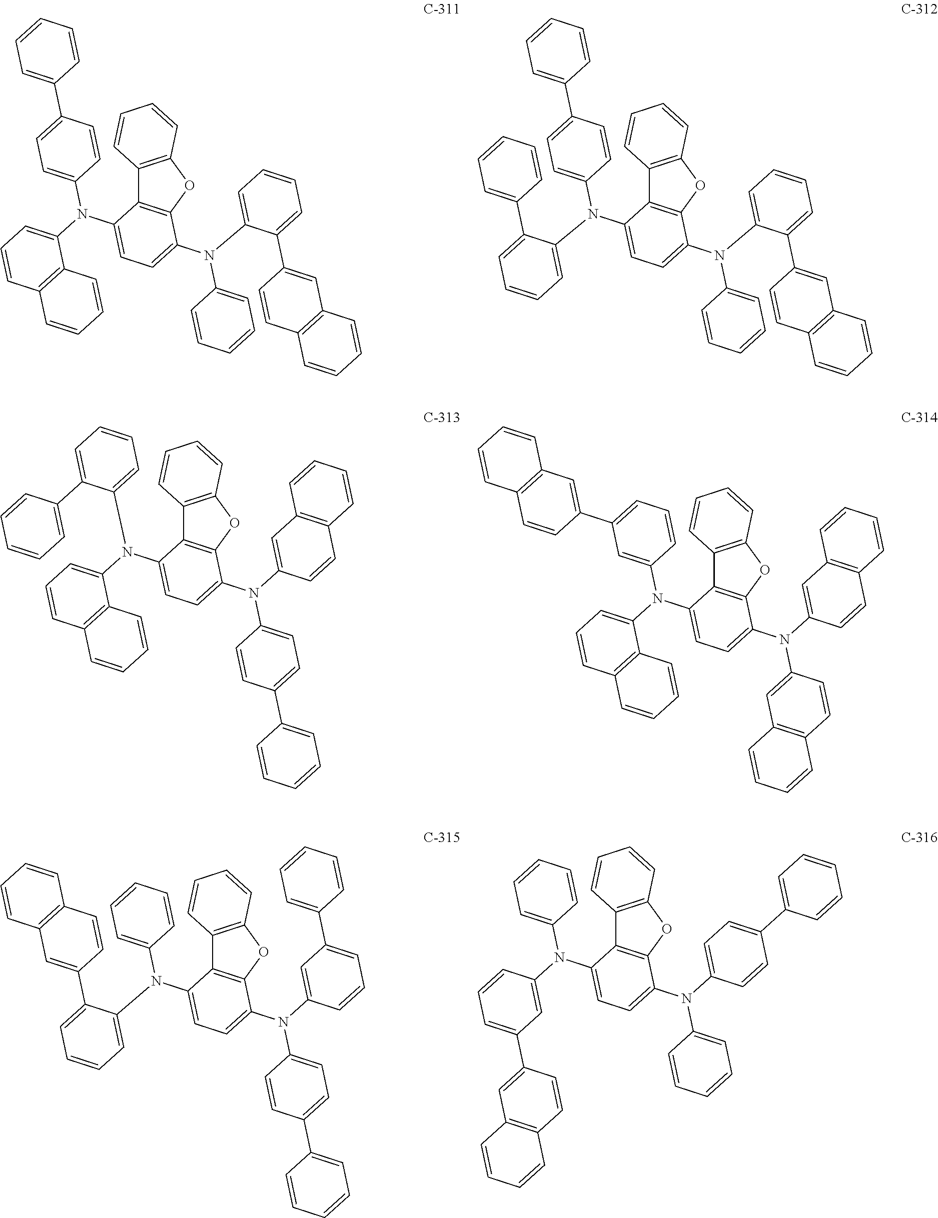 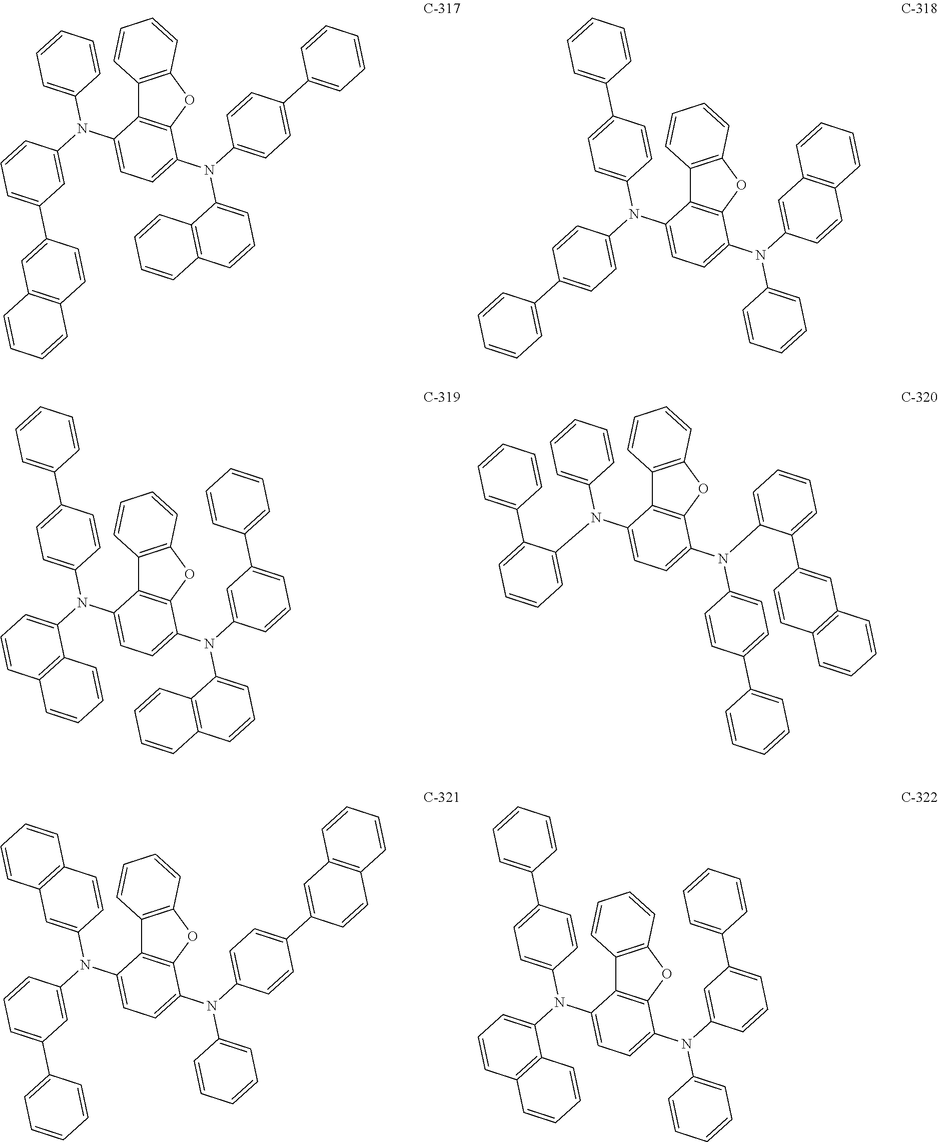 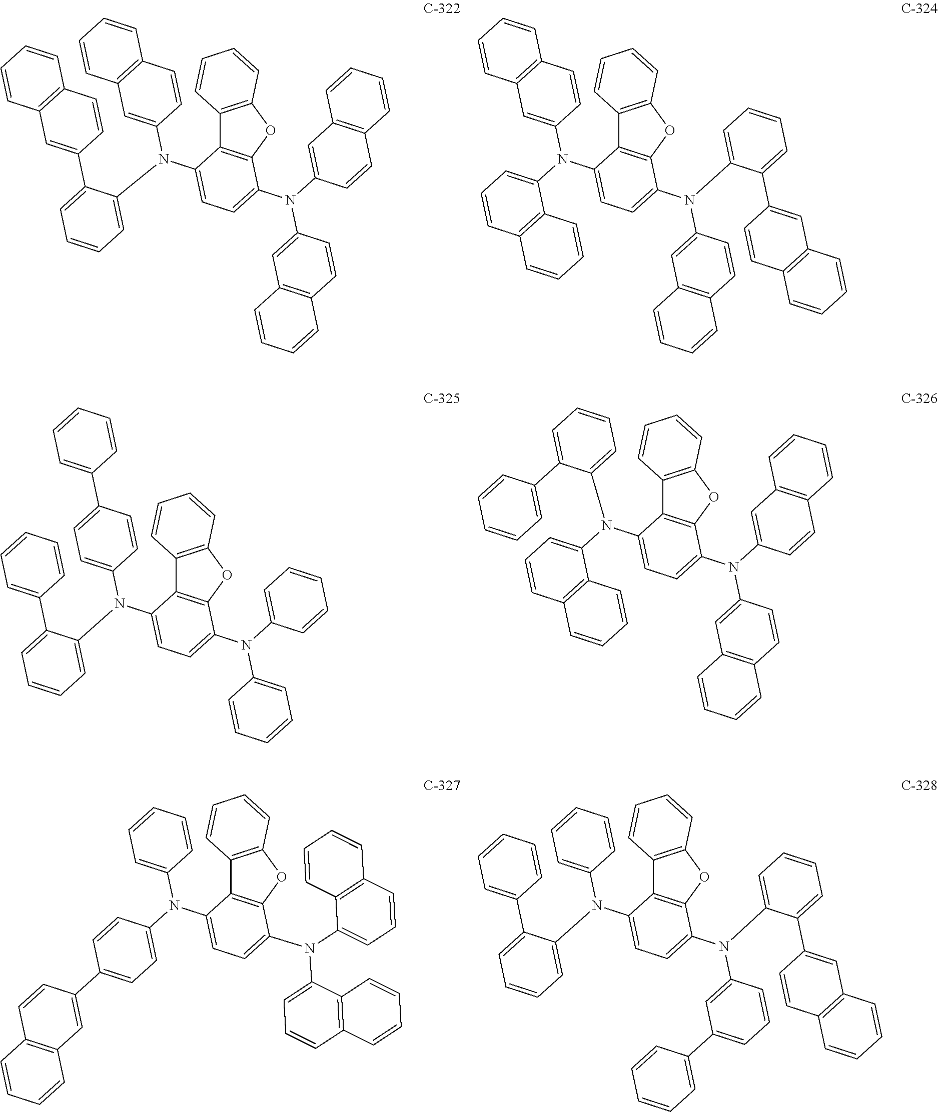 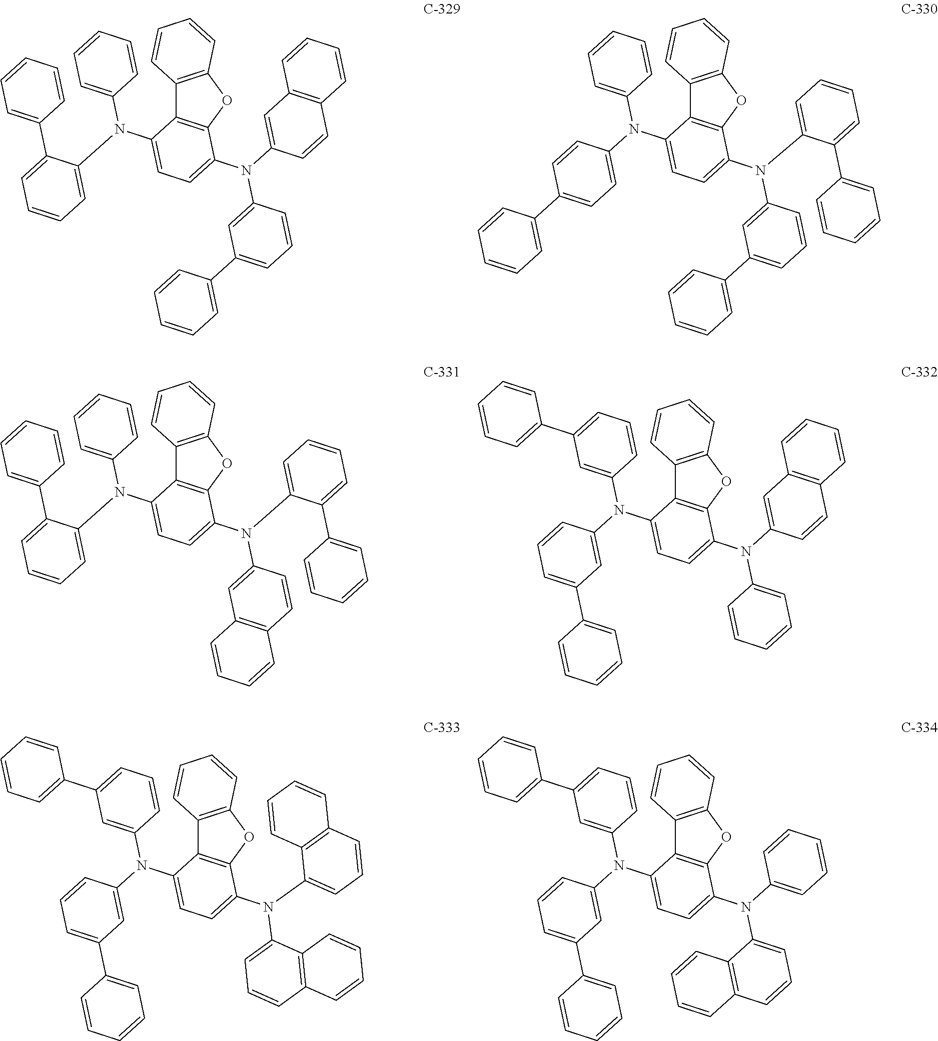 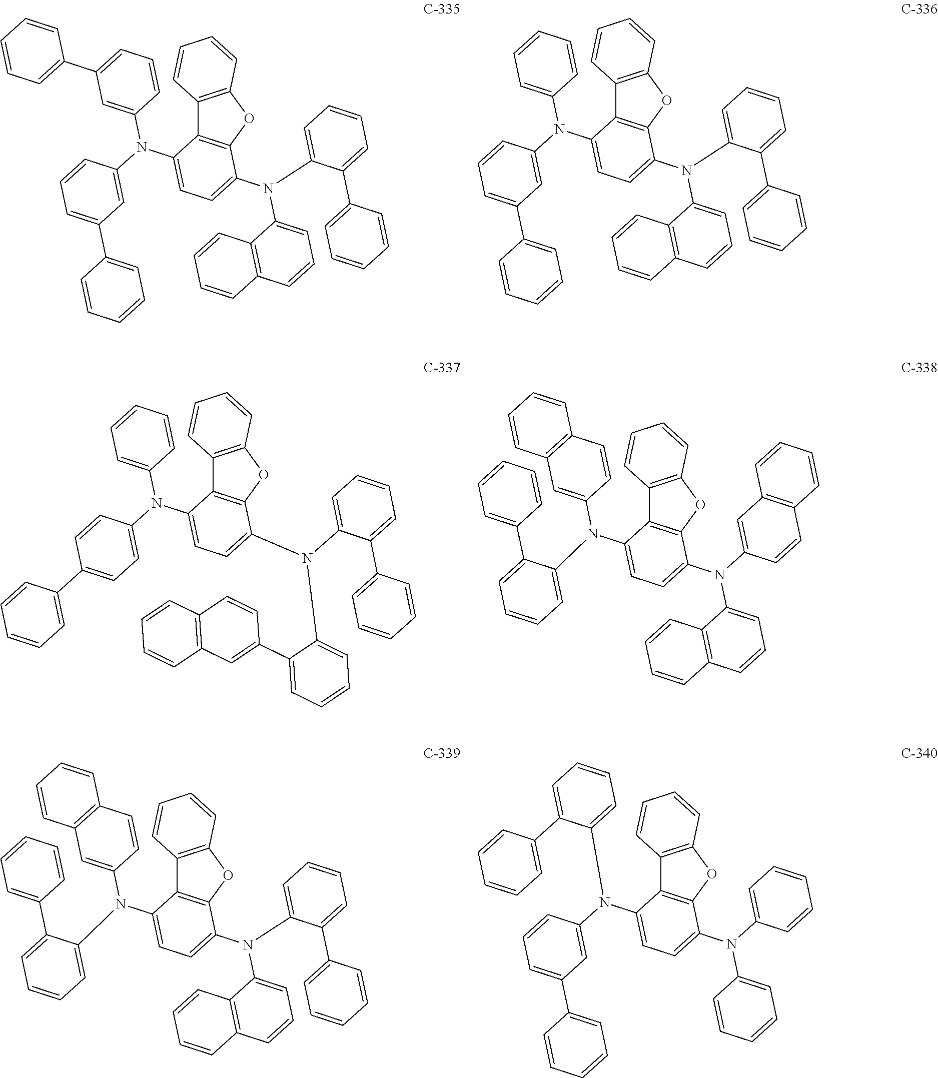 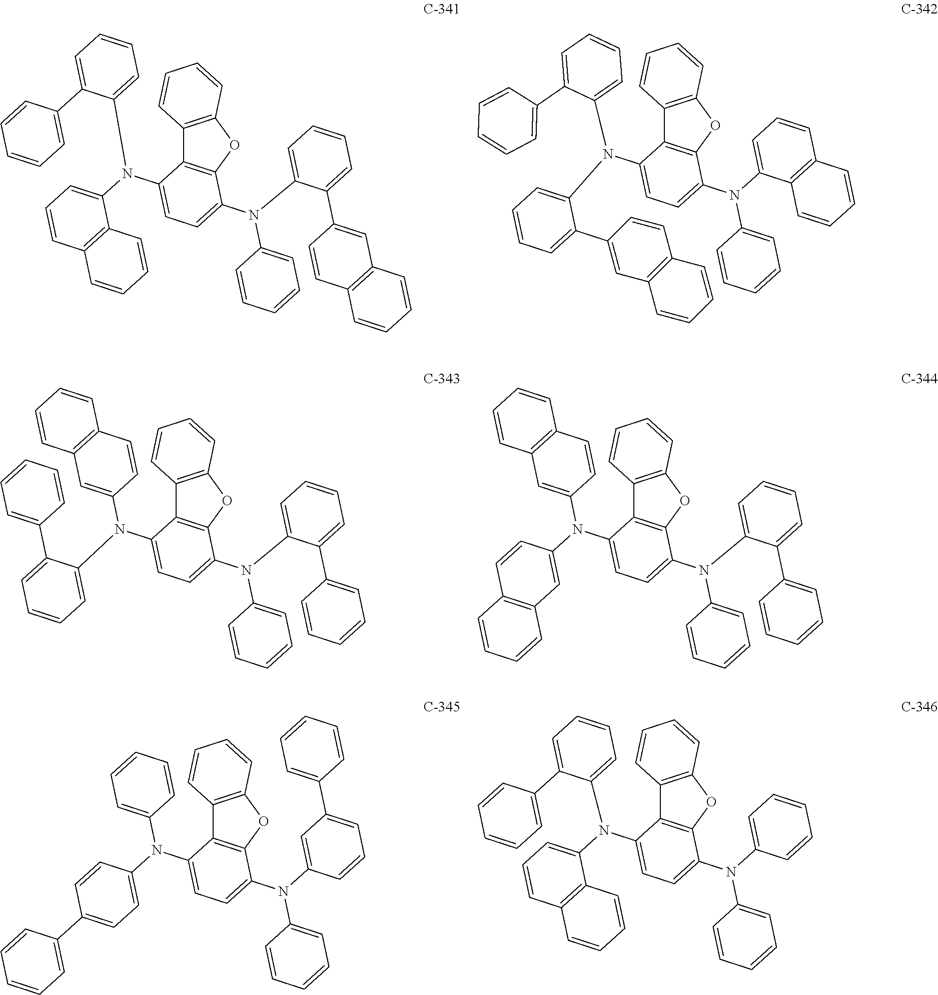 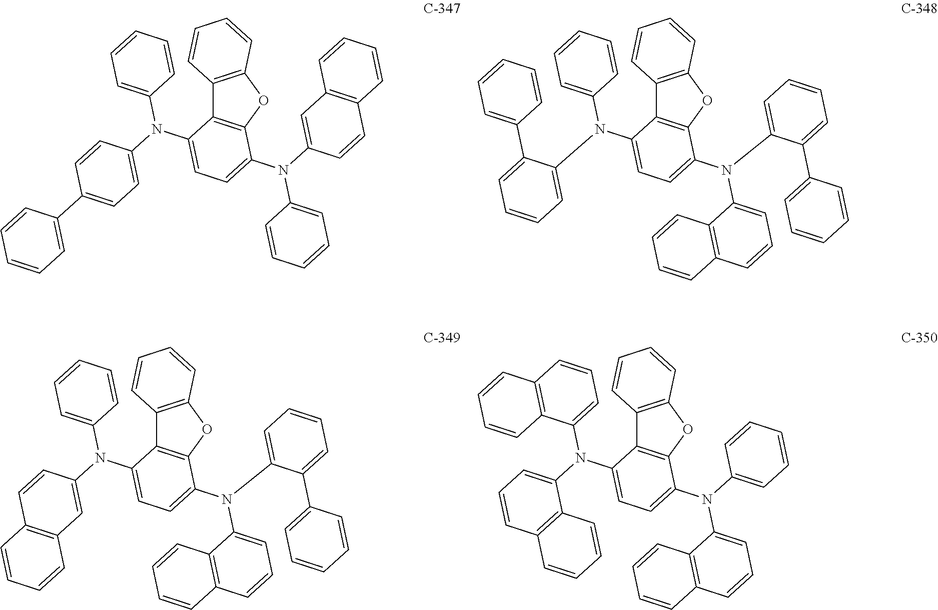 [SMILES: c1ccc(-c2ccc(N(c3ccccc3)c3ccc(N(c4ccccc4)c4ccc5ccccc5c4)c4oc5ccccc5c34)cc2)cc1 ; c1ccc(-c2ccccc2N(c2cccc3ccccc23)c2ccc(N(c3ccccc3)c3ccccc3-c3ccccc3)c3c2oc2ccccc23)cc1 ; c1ccc(-c2ccccc2N(c2cccc3ccccc23)c2ccc(N(c3ccccc3)c3ccc4ccccc4c3)c3c2oc2ccccc23)cc1 ; c1ccc(N(c2cccc3ccccc23)c2ccc(N(c3cccc4ccccc34)c3cccc4ccccc34)c3c2oc2ccccc23)cc1] 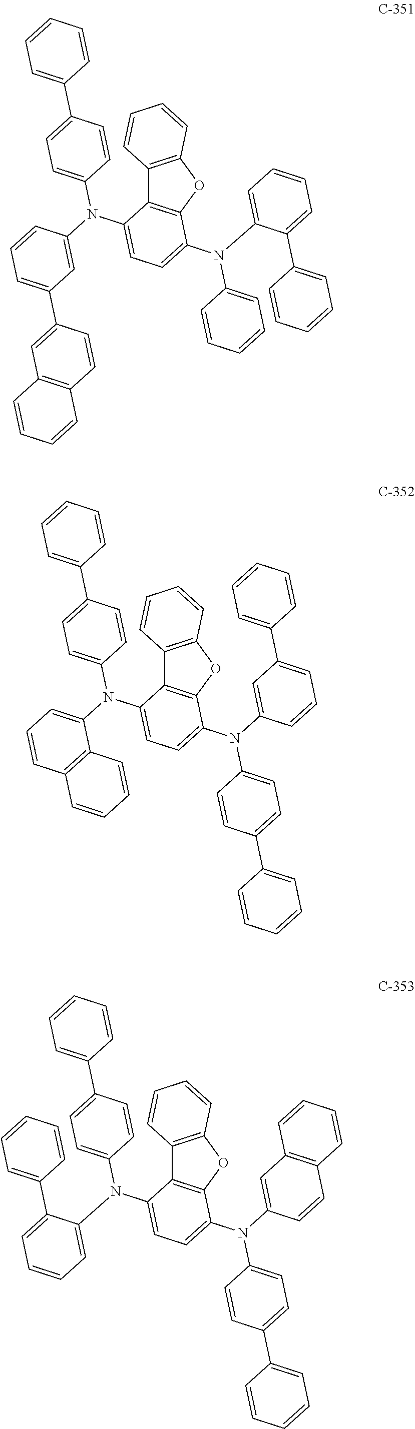 [SMILES: c1ccc(-c2ccc(N(c3cccc(-c4ccc5ccccc5c4)c3)c3ccc(N(c4ccccc4)c4ccccc4-c4ccccc4)c4oc5ccccc5c34)cc2)cc1 ; c1ccc(-c2ccc(N(c3cccc(-c4ccccc4)c3)c3ccc(N(c4ccc(-c5ccccc5)cc4)c4cccc5ccccc45)c4c3oc3ccccc34)cc2)cc1 ; c1ccc(-c2ccc(N(c3ccc4ccccc4c3)c3ccc(N(c4ccc(-c5ccccc5)cc4)c4ccccc4-c4ccccc4)c4c3oc3ccccc34)cc2)cc1] 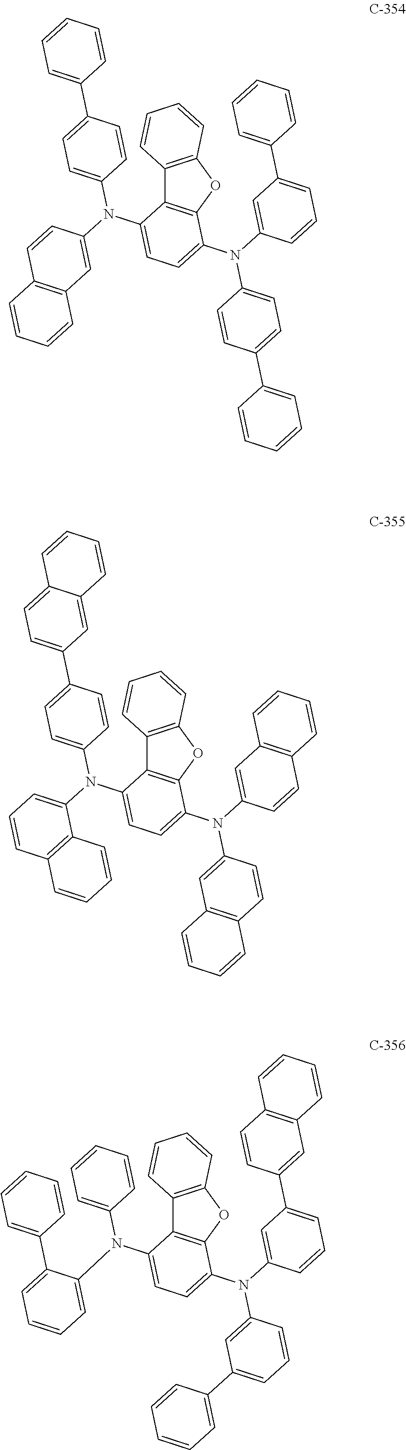 [SMILES: c1ccc(-c2ccc(N(c3cccc(-c4ccccc4)c3)c3ccc(N(c4ccc(-c5ccccc5)cc4)c4ccc5ccccc5c4)c4c3oc3ccccc34)cc2)cc1 ; c1ccc2cc(-c3ccc(N(c4cccc5ccccc45)c4ccc(N(c5ccc6ccccc6c5)c5ccc6ccccc6c5)c5oc6ccccc6c45)cc3)ccc2c1 ; c1ccc(-c2cccc(N(c3cccc(-c4ccc5ccccc5c4)c3)c3ccc(N(c4ccccc4)c4ccccc4-c4ccccc4)c4c3oc3ccccc34)c2)cc1] 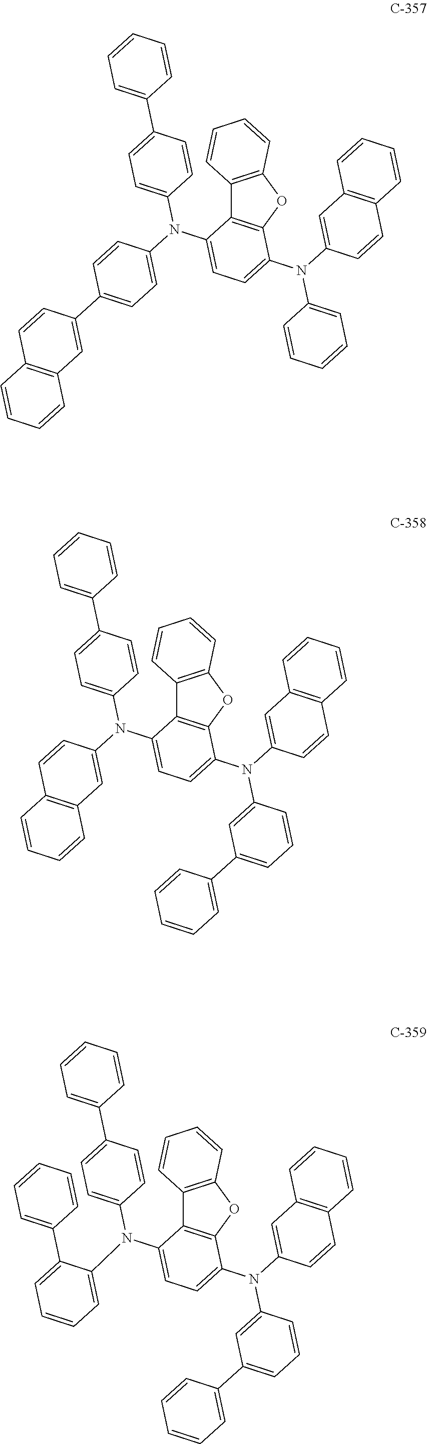 [SMILES: c1ccc(-c2ccc(N(c3ccc(-c4ccc5ccccc5c4)cc3)c3ccc(N(c4ccccc4)c4ccc5ccccc5c4)c4oc5ccccc5c34)cc2)cc1 ; c1ccc(-c2ccc(N(c3ccc4ccccc4c3)c3ccc(N(c4cccc(-c5ccccc5)c4)c4ccc5ccccc5c4)c4oc5ccccc5c34)cc2)cc1 ; c1ccc(-c2ccc(N(c3ccccc3-c3ccccc3)c3ccc(N(c4cccc(-c5ccccc5)c4)c4ccc5ccccc5c4)c4oc5ccccc5c34)cc2)cc1] 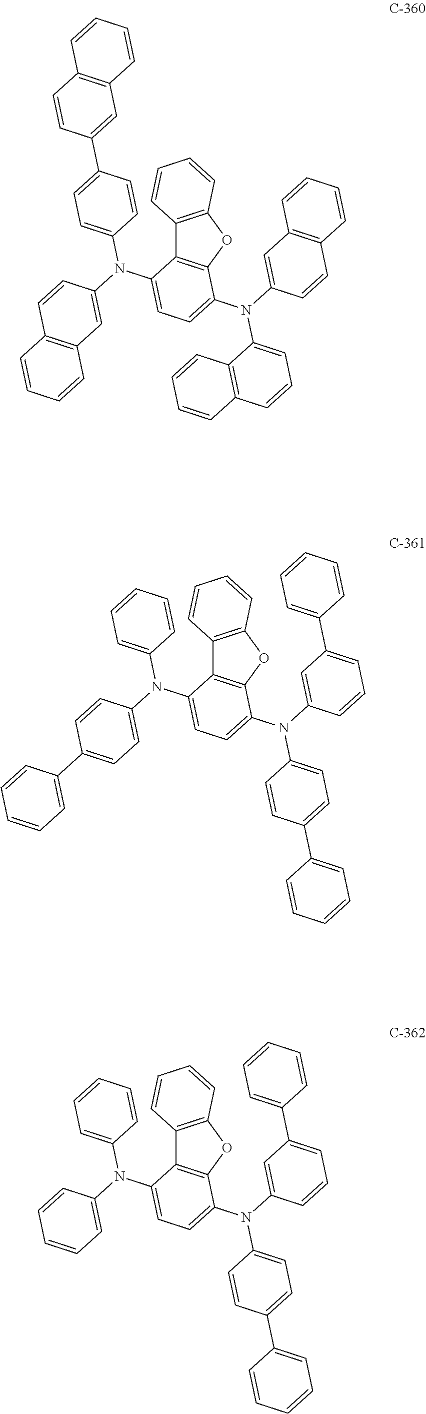 [SMILES: c1ccc2cc(-c3ccc(N(c4ccc5ccccc5c4)c4ccc(N(c5ccc6ccccc6c5)c5cccc6ccccc56)c5oc6ccccc6c45)cc3)ccc2c1 ; c1ccc(-c2ccc(N(c3cccc(-c4ccccc4)c3)c3ccc(N(c4ccccc4)c4ccc(-c5ccccc5)cc4)c4c3oc3ccccc34)cc2)cc1 ; c1ccc(-c2ccc(N(c3cccc(-c4ccccc4)c3)c3ccc(N(c4ccccc4)c4ccccc4)c4c3oc3ccccc34)cc2)cc1] 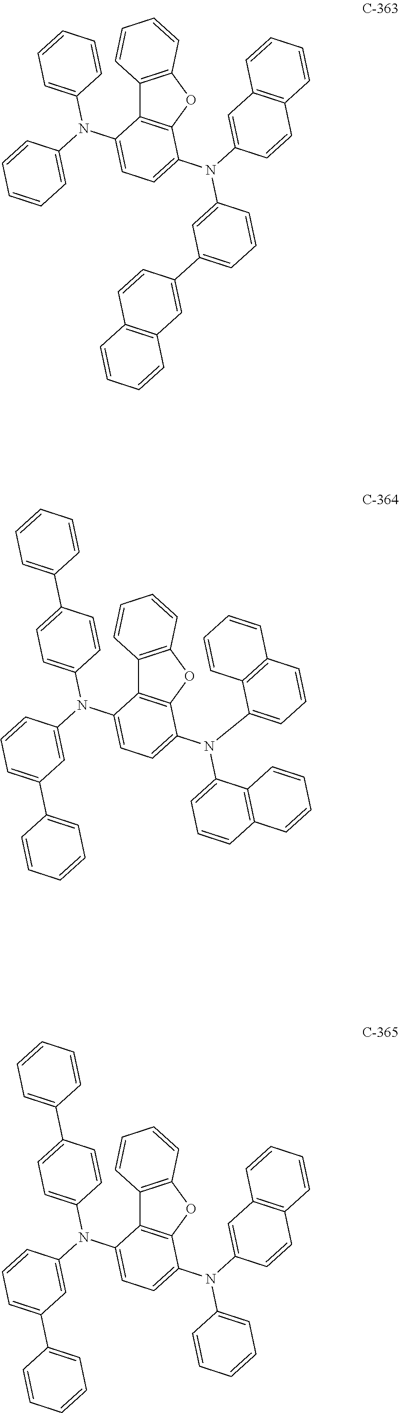 [SMILES: c1ccc(N(c2ccccc2)c2ccc(N(c3cccc(-c4ccc5ccccc5c4)c3)c3ccc4ccccc4c3)c3oc4ccccc4c23)cc1 ; c1ccc(-c2ccc(N(c3cccc(-c4ccccc4)c3)c3ccc(N(c4cccc5ccccc45)c4cccc5ccccc45)c4oc5ccccc5c34)cc2)cc1 ; c1ccc(-c2ccc(N(c3cccc(-c4ccccc4)c3)c3ccc(N(c4ccccc4)c4ccc5ccccc5c4)c4oc5ccccc5c34)cc2)cc1] 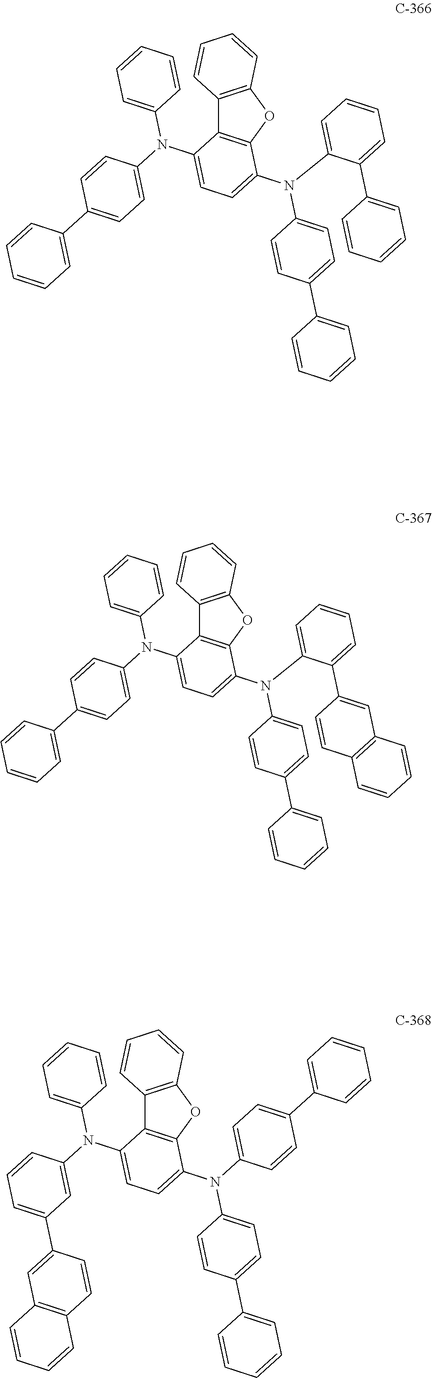 [SMILES: c1ccc(-c2ccc(N(c3ccccc3-c3ccccc3)c3ccc(N(c4ccccc4)c4ccc(-c5ccccc5)cc4)c4c3oc3ccccc34)cc2)cc1 ; c1ccc(-c2ccc(N(c3ccccc3-c3ccc4ccccc4c3)c3ccc(N(c4ccccc4)c4ccc(-c5ccccc5)cc4)c4c3oc3ccccc34)cc2)cc1 ; c1ccc(-c2ccc(N(c3ccc(-c4ccccc4)cc3)c3ccc(N(c4ccccc4)c4cccc(-c5ccc6ccccc6c5)c4)c4c3oc3ccccc34)cc2)cc1] 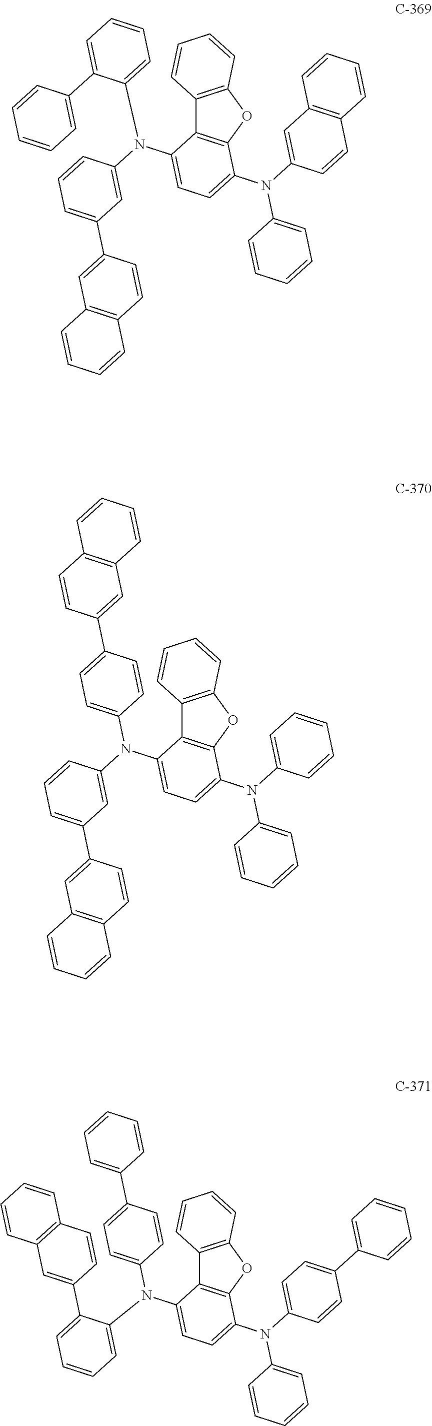 [SMILES: c1ccc(-c2ccccc2N(c2cccc(-c3ccc4ccccc4c3)c2)c2ccc(N(c3ccccc3)c3ccc4ccccc4c3)c3oc4ccccc4c23)cc1 ; c1ccc(N(c2ccccc2)c2ccc(N(c3ccc(-c4ccc5ccccc5c4)cc3)c3cccc(-c4ccc5ccccc5c4)c3)c3c2oc2ccccc23)cc1 ; c1ccc(-c2ccc(N(c3ccccc3)c3ccc(N(c4ccc(-c5ccccc5)cc4)c4ccccc4-c4ccc5ccccc5c4)c4c3oc3ccccc34)cc2)cc1] 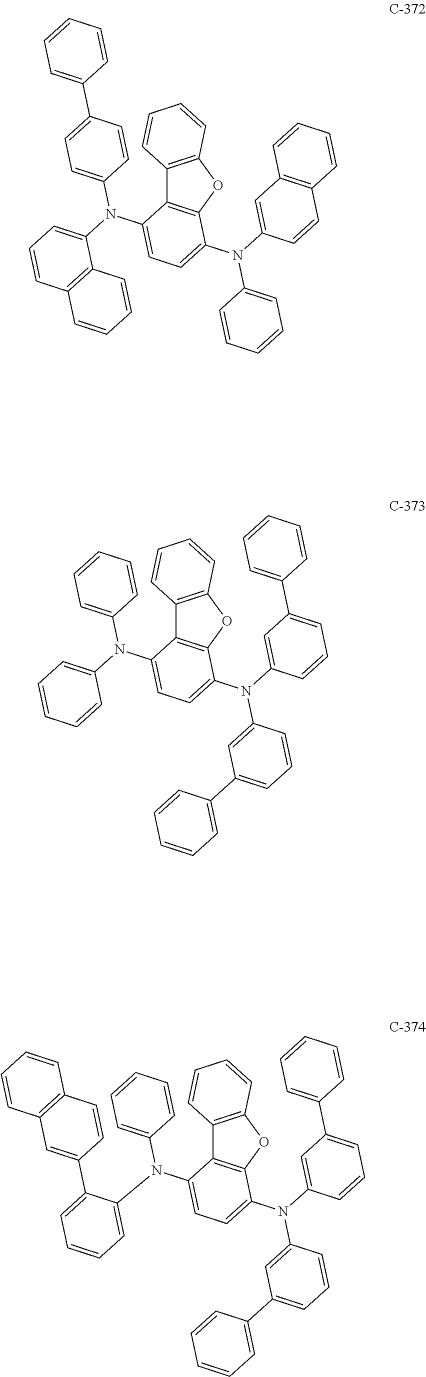 [SMILES: c1ccc(-c2ccc(N(c3cccc4ccccc34)c3ccc(N(c4ccccc4)c4ccc5ccccc5c4)c4oc5ccccc5c34)cc2)cc1 ; c1ccc(-c2cccc(N(c3cccc(-c4ccccc4)c3)c3ccc(N(c4ccccc4)c4ccccc4)c4c3oc3ccccc34)c2)cc1 ; c1ccc(-c2cccc(N(c3cccc(-c4ccccc4)c3)c3ccc(N(c4ccccc4)c4ccccc4-c4ccc5ccccc5c4)c4c3oc3ccccc34)c2)cc1] 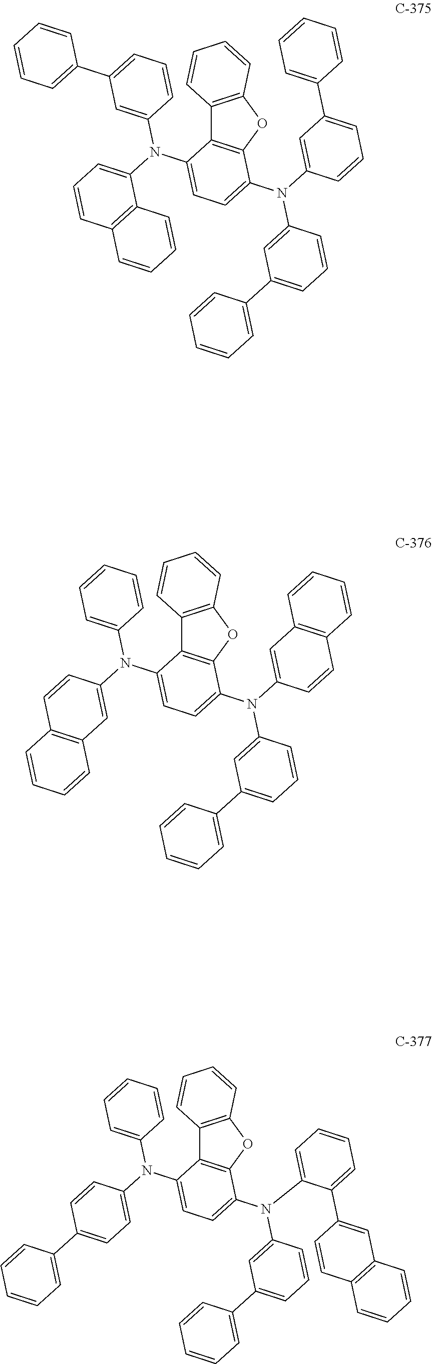 [SMILES: c1ccc(-c2cccc(N(c3cccc(-c4ccccc4)c3)c3ccc(N(c4cccc(-c5ccccc5)c4)c4cccc5ccccc45)c4c3oc3ccccc34)c2)cc1 ; c1ccc(-c2cccc(N(c3ccc4ccccc4c3)c3ccc(N(c4ccccc4)c4ccc5ccccc5c4)c4c3oc3ccccc34)c2)cc1 ; c1ccc(-c2ccc(N(c3ccccc3)c3ccc(N(c4cccc(-c5ccccc5)c4)c4ccccc4-c4ccc5ccccc5c4)c4oc5ccccc5c34)cc2)cc1] 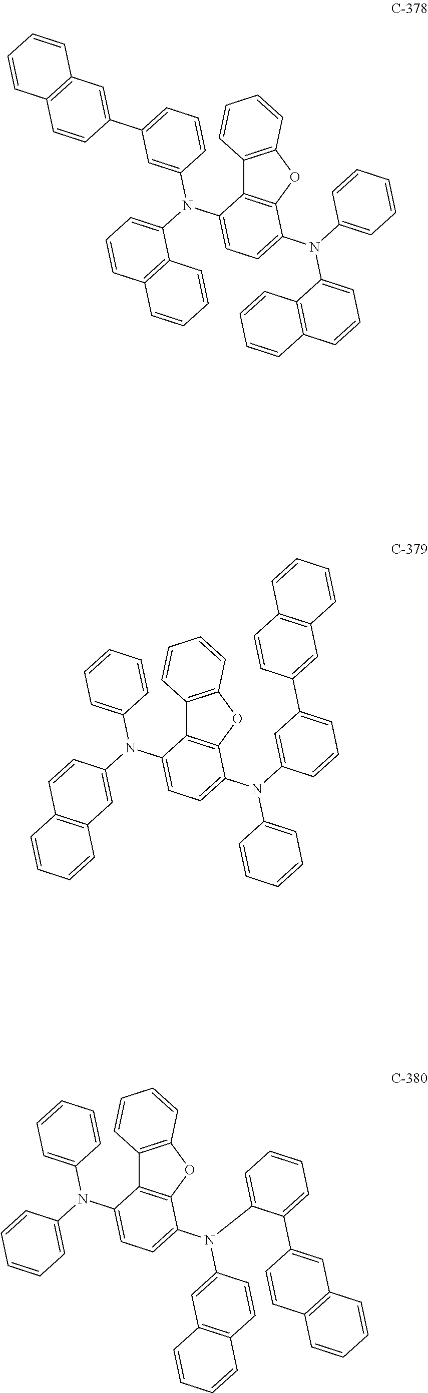 [SMILES: c1ccc(N(c2cccc3ccccc23)c2ccc(N(c3cccc(-c4ccc5ccccc5c4)c3)c3cccc4ccccc34)c3c2oc2ccccc23)cc1 ; c1ccc(N(c2cccc(-c3ccc4ccccc4c3)c2)c2ccc(N(c3ccccc3)c3ccc4ccccc4c3)c3c2oc2ccccc23)cc1 ; c1ccc(N(c2ccccc2)c2ccc(N(c3ccc4ccccc4c3)c3ccccc3-c3ccc4ccccc4c3)c3oc4ccccc4c23)cc1] 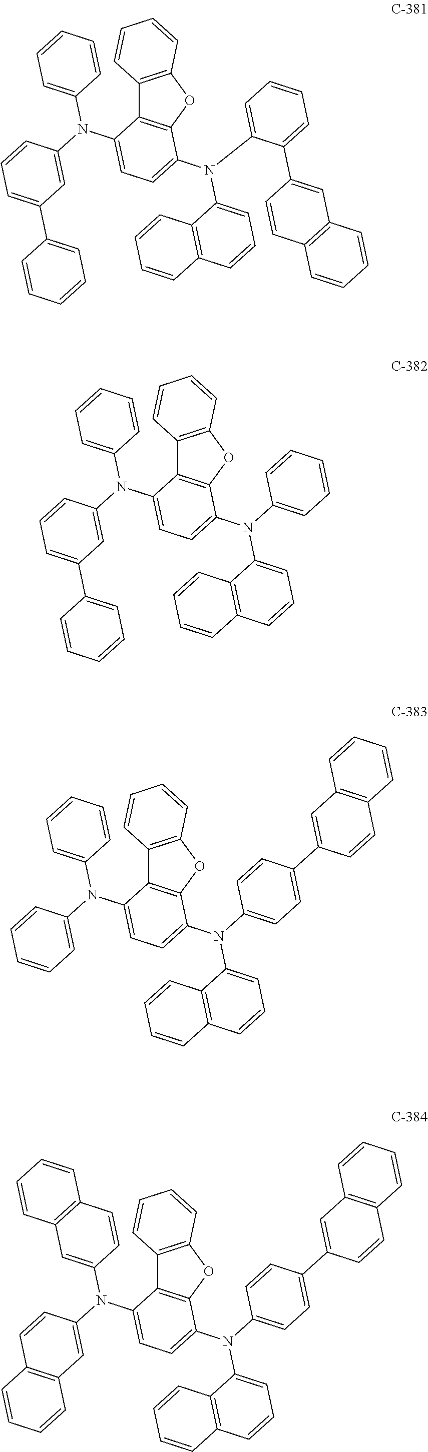 [SMILES: c1ccc(-c2cccc(N(c3ccccc3)c3ccc(N(c4ccccc4-c4ccc5ccccc5c4)c4cccc5ccccc45)c4oc5ccccc5c34)c2)cc1 ; c1ccc(-c2cccc(N(c3ccccc3)c3ccc(N(c4ccccc4)c4cccc5ccccc45)c4oc5ccccc5c34)c2)cc1 ; c1ccc(N(c2ccccc2)c2ccc(N(c3ccc(-c4ccc5ccccc5c4)cc3)c3cccc4ccccc34)c3oc4ccccc4c23)cc1 ; c1ccc2cc(-c3ccc(N(c4cccc5ccccc45)c4ccc(N(c5ccc6ccccc6c5)c5ccc6ccccc6c5)c5c4oc4ccccc45)cc3)ccc2c1] 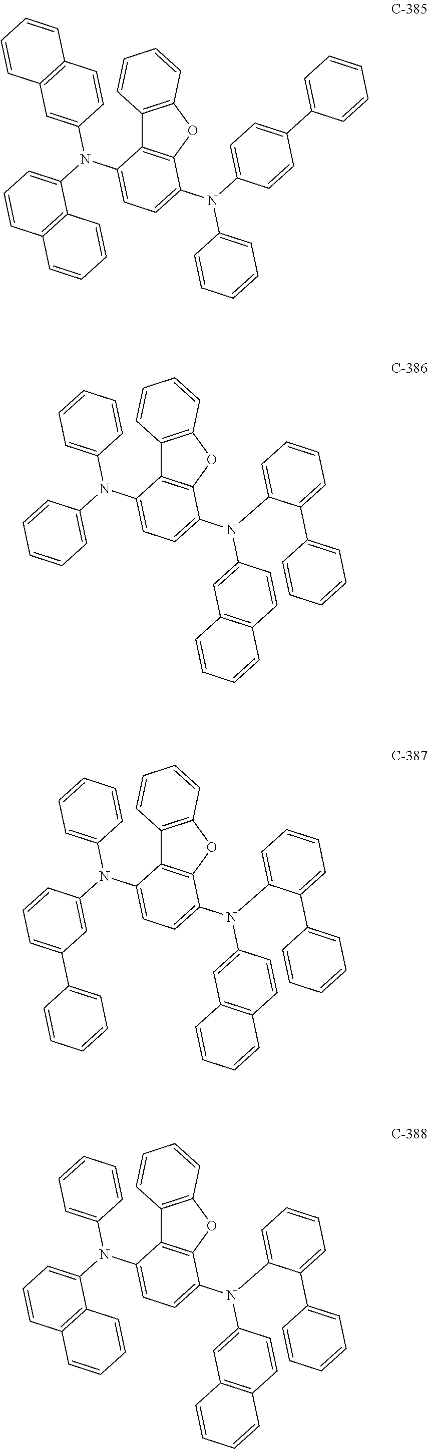 [SMILES: c1ccc(-c2ccc(N(c3ccccc3)c3ccc(N(c4ccc5ccccc5c4)c4cccc5ccccc45)c4c3oc3ccccc34)cc2)cc1 ; c1ccc(-c2ccccc2N(c2ccc3ccccc3c2)c2ccc(N(c3ccccc3)c3ccccc3)c3c2oc2ccccc23)cc1 ; c1ccc(-c2cccc(N(c3ccccc3)c3ccc(N(c4ccc5ccccc5c4)c4ccccc4-c4ccccc4)c4oc5ccccc5c34)c2)cc1 ; c1ccc(-c2ccccc2N(c2ccc3ccccc3c2)c2ccc(N(c3ccccc3)c3cccc4ccccc34)c3c2oc2ccccc23)cc1] 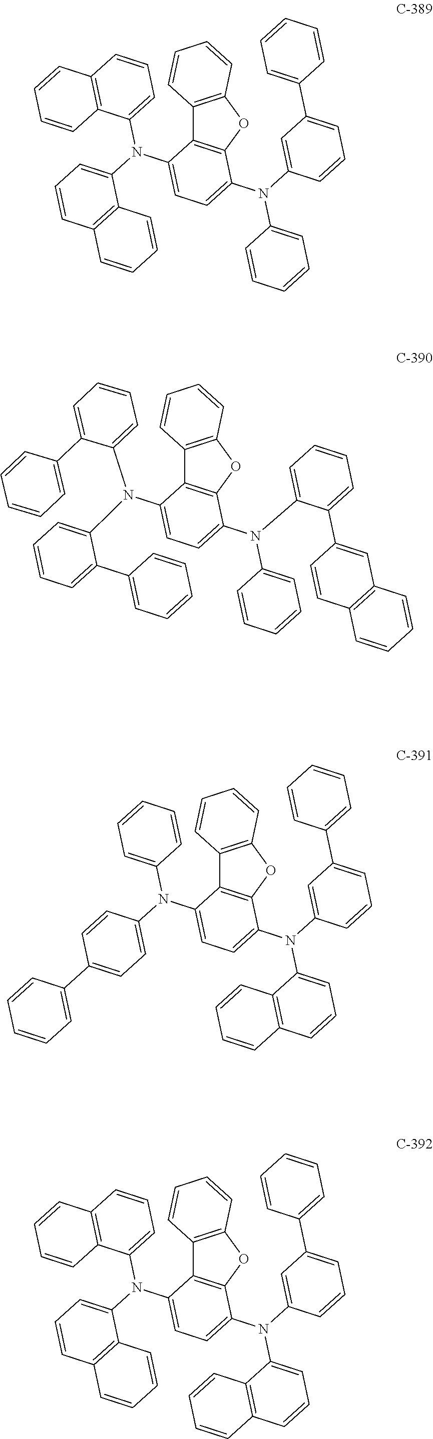 [SMILES: c1ccc(-c2cccc(N(c3ccccc3)c3ccc(N(c4cccc5ccccc45)c4cccc5ccccc45)c4c3oc3ccccc34)c2)cc1 ; c1ccc(-c2ccccc2N(c2ccccc2-c2ccccc2)c2ccc(N(c3ccccc3)c3ccccc3-c3ccc4ccccc4c3)c3oc4ccccc4c23)cc1 ; c1ccc(-c2ccc(N(c3ccccc3)c3ccc(N(c4cccc(-c5ccccc5)c4)c4cccc5ccccc45)c4oc5ccccc5c34)cc2)cc1 ; c1ccc(-c2cccc(N(c3cccc4ccccc34)c3ccc(N(c4cccc5ccccc45)c4cccc5ccccc45)c4c3oc3ccccc34)c2)cc1] 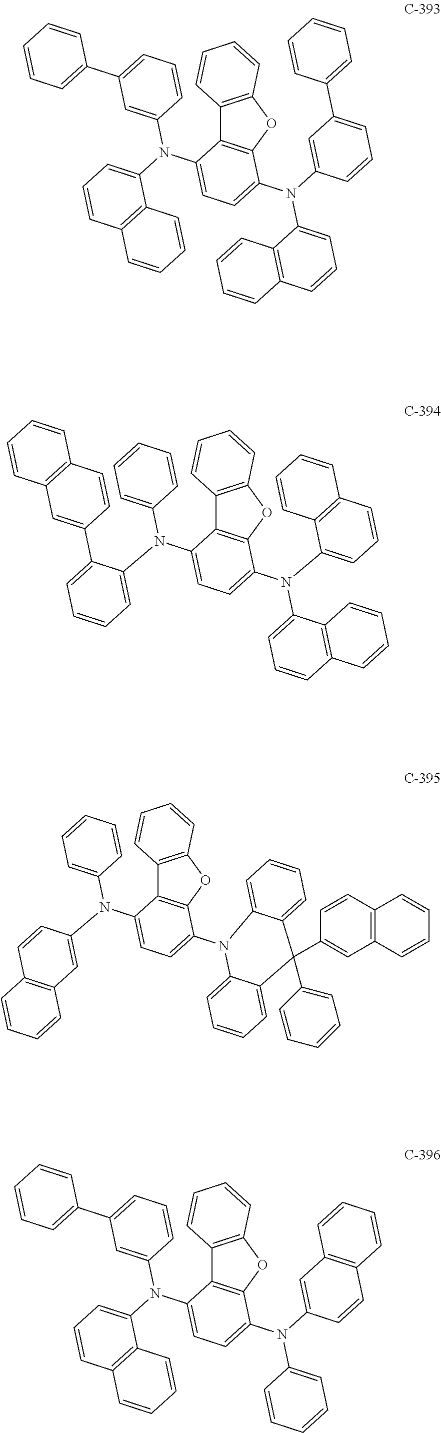 [SMILES: c1ccc(-c2cccc(N(c3cccc4ccccc34)c3ccc(N(c4cccc(-c5ccccc5)c4)c4cccc5ccccc45)c4c3oc3ccccc34)c2)cc1 ; c1ccc(N(c2ccccc2-c2ccc3ccccc3c2)c2ccc(N(c3cccc4ccccc34)c3cccc4ccccc34)c3oc4ccccc4c23)cc1 ; c1ccc(N(c2ccc3ccccc3c2)c2ccc(N3c4ccccc4C(c4ccccc4)(c4ccc5ccccc5c4)c4ccccc43)c3oc4ccccc4c23)cc1 ; c1ccc(-c2cccc(N(c3cccc4ccccc34)c3ccc(N(c4ccccc4)c4ccc5ccccc5c4)c4oc5ccccc5c34)c2)cc1] 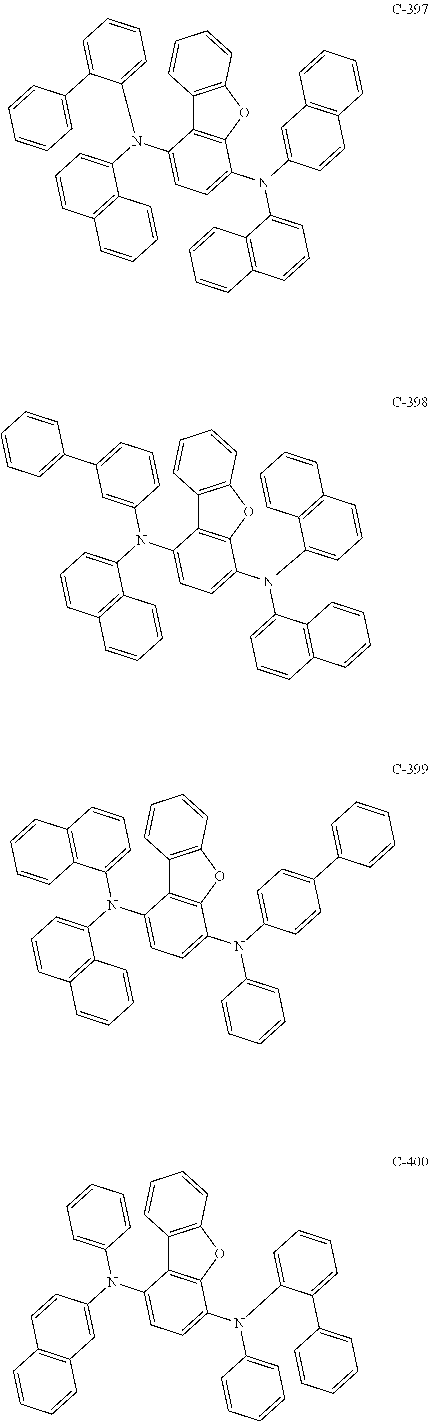 [SMILES: c1ccc(-c2ccccc2N(c2cccc3ccccc23)c2ccc(N(c3ccc4ccccc4c3)c3cccc4ccccc34)c3oc4ccccc4c23)cc1 ; c1ccc(-c2cccc(N(c3cccc4ccccc34)c3ccc(N(c4cccc5ccccc45)c4cccc5ccccc45)c4oc5ccccc5c34)c2)cc1 ; c1ccc(-c2ccc(N(c3ccccc3)c3ccc(N(c4cccc5ccccc45)c4cccc5ccccc45)c4c3oc3ccccc34)cc2)cc1 ; c1ccc(-c2ccccc2N(c2ccccc2)c2ccc(N(c3ccccc3)c3ccc4ccccc4c3)c3c2oc2ccccc23)cc1] 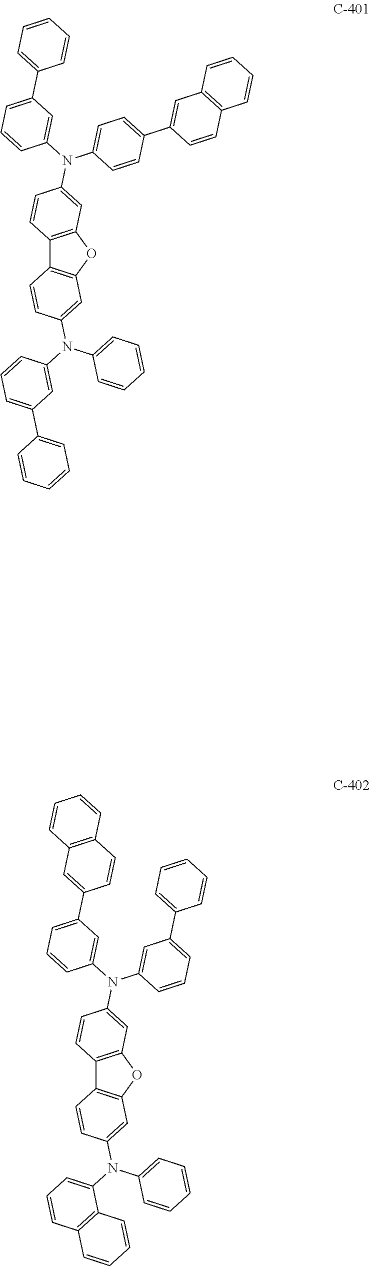 [SMILES: c1ccc(-c2cccc(N(c3ccccc3)c3ccc4c(c3)oc3cc(N(c5ccc(-c6ccc7ccccc7c6)cc5)c5cccc(-c6ccccc6)c5)ccc34)c2)cc1 ; c1ccc(-c2cccc(N(c3cccc(-c4ccc5ccccc5c4)c3)c3ccc4c(c3)oc3cc(N(c5ccccc5)c5cccc6ccccc56)ccc34)c2)cc1] 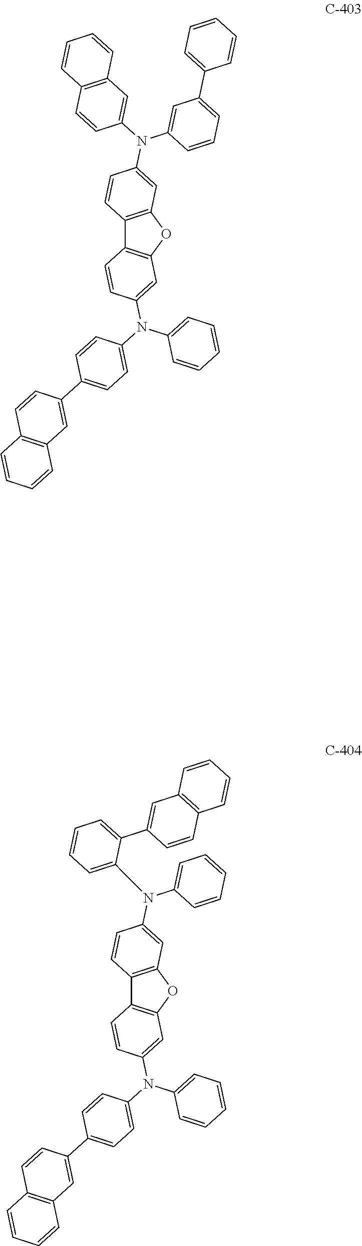 [SMILES: c1ccc(-c2cccc(N(c3ccc4ccccc4c3)c3ccc4c(c3)oc3cc(N(c5ccccc5)c5ccc(-c6ccc7ccccc7c6)cc5)ccc34)c2)cc1 ; c1ccc(N(c2ccc(-c3ccc4ccccc4c3)cc2)c2ccc3c(c2)oc2cc(N(c4ccccc4)c4ccccc4-c4ccc5ccccc5c4)ccc23)cc1] 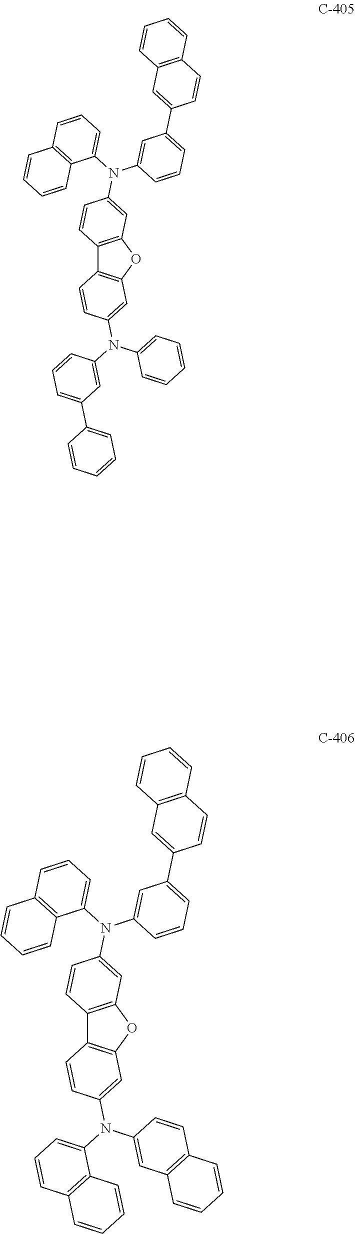 [SMILES: c1ccc(-c2cccc(N(c3ccccc3)c3ccc4c(c3)oc3cc(N(c5cccc(-c6ccc7ccccc7c6)c5)c5cccc6ccccc56)ccc34)c2)cc1 ; c1cc(-c2ccc3ccccc3c2)cc(N(c2ccc3c(c2)oc2cc(N(c4ccc5ccccc5c4)c4cccc5ccccc45)ccc23)c2cccc3ccccc23)c1] 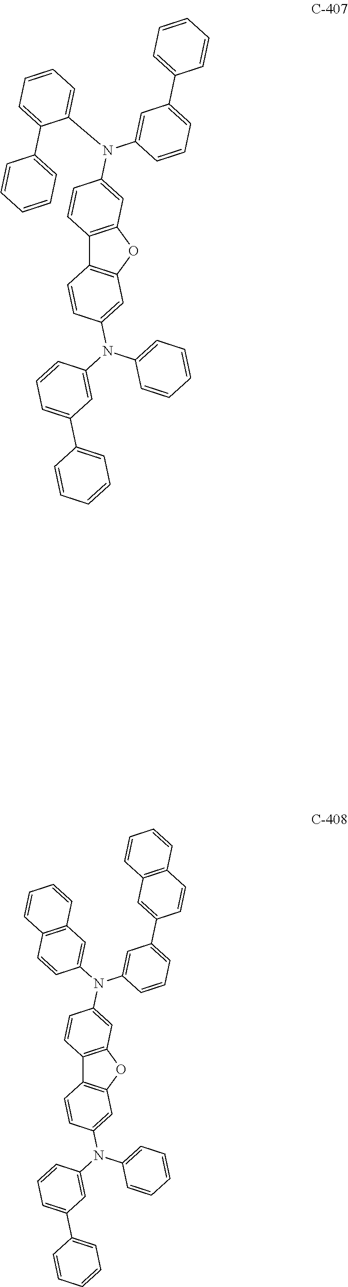 [SMILES: c1ccc(-c2cccc(N(c3ccccc3)c3ccc4c(c3)oc3cc(N(c5cccc(-c6ccccc6)c5)c5ccccc5-c5ccccc5)ccc34)c2)cc1 ; c1ccc(-c2cccc(N(c3ccccc3)c3ccc4c(c3)oc3cc(N(c5cccc(-c6ccc7ccccc7c6)c5)c5ccc6ccccc6c5)ccc34)c2)cc1] 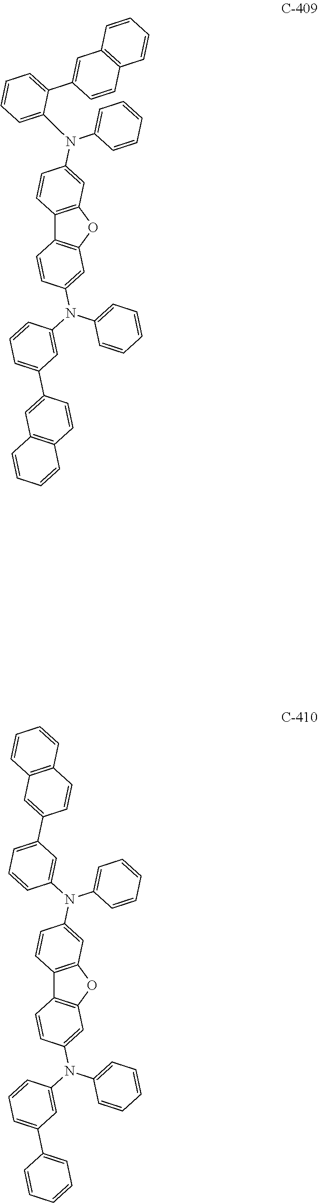 [SMILES: c1ccc(N(c2cccc(-c3ccc4ccccc4c3)c2)c2ccc3c(c2)oc2cc(N(c4ccccc4)c4ccccc4-c4ccc5ccccc5c4)ccc23)cc1 ; c1ccc(-c2cccc(N(c3ccccc3)c3ccc4c(c3)oc3cc(N(c5ccccc5)c5cccc(-c6ccc7ccccc7c6)c5)ccc34)c2)cc1] 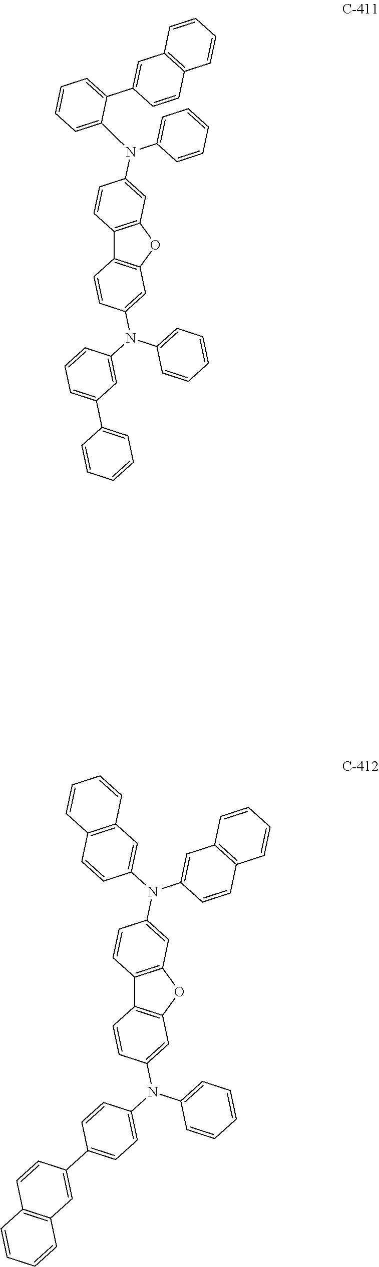 [SMILES: c1ccc(-c2cccc(N(c3ccccc3)c3ccc4c(c3)oc3cc(N(c5ccccc5)c5ccccc5-c5ccc6ccccc6c5)ccc34)c2)cc1 ; c1ccc(N(c2ccc(-c3ccc4ccccc4c3)cc2)c2ccc3c(c2)oc2cc(N(c4ccc5ccccc5c4)c4ccc5ccccc5c4)ccc23)cc1] 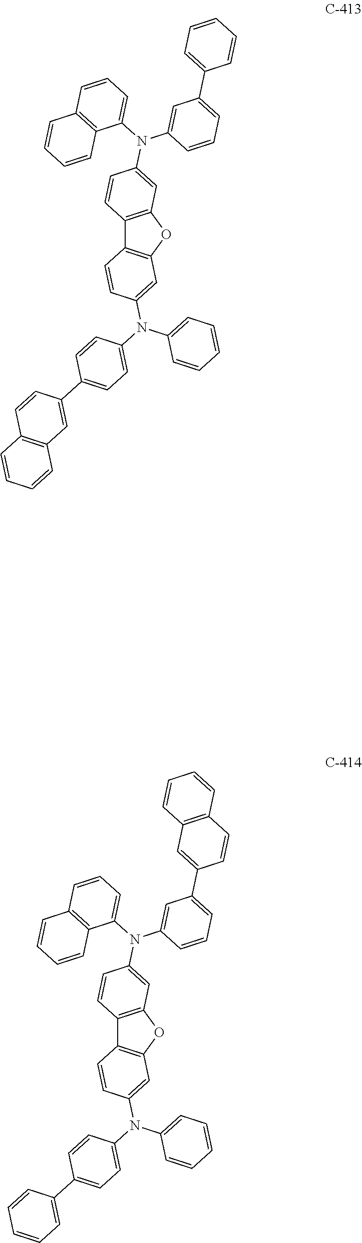 [SMILES: c1ccc(-c2cccc(N(c3ccc4c(c3)oc3cc(N(c5ccccc5)c5ccc(-c6ccc7ccccc7c6)cc5)ccc34)c3cccc4ccccc34)c2)cc1 ; c1ccc(-c2ccc(N(c3ccccc3)c3ccc4c(c3)oc3cc(N(c5cccc(-c6ccc7ccccc7c6)c5)c5cccc6ccccc56)ccc34)cc2)cc1] 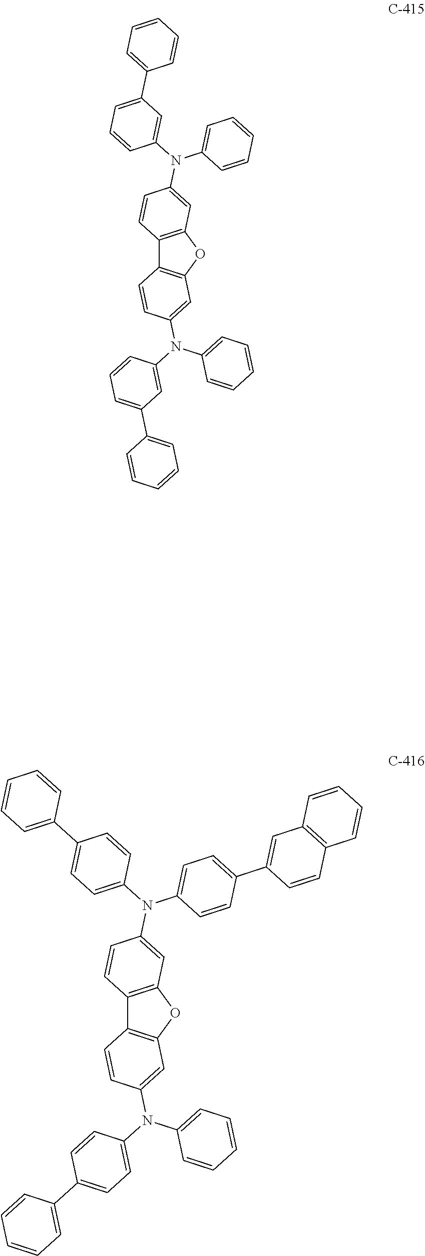 [SMILES: c1ccc(-c2cccc(N(c3ccccc3)c3ccc4c(c3)oc3cc(N(c5ccccc5)c5cccc(-c6ccccc6)c5)ccc34)c2)cc1 ; c1ccc(-c2ccc(N(c3ccccc3)c3ccc4c(c3)oc3cc(N(c5ccc(-c6ccccc6)cc5)c5ccc(-c6ccc7ccccc7c6)cc5)ccc34)cc2)cc1] 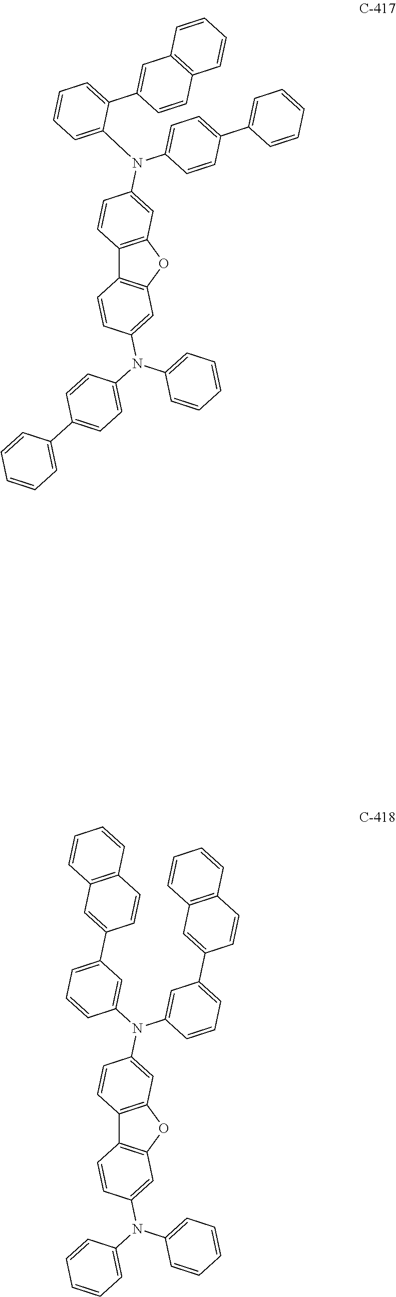 [SMILES: c1ccc(-c2ccc(N(c3ccccc3)c3ccc4c(c3)oc3cc(N(c5ccc(-c6ccccc6)cc5)c5ccccc5-c5ccc6ccccc6c5)ccc34)cc2)cc1 ; c1ccc(N(c2ccccc2)c2ccc3c(c2)oc2cc(N(c4cccc(-c5ccc6ccccc6c5)c4)c4cccc(-c5ccc6ccccc6c5)c4)ccc23)cc1] 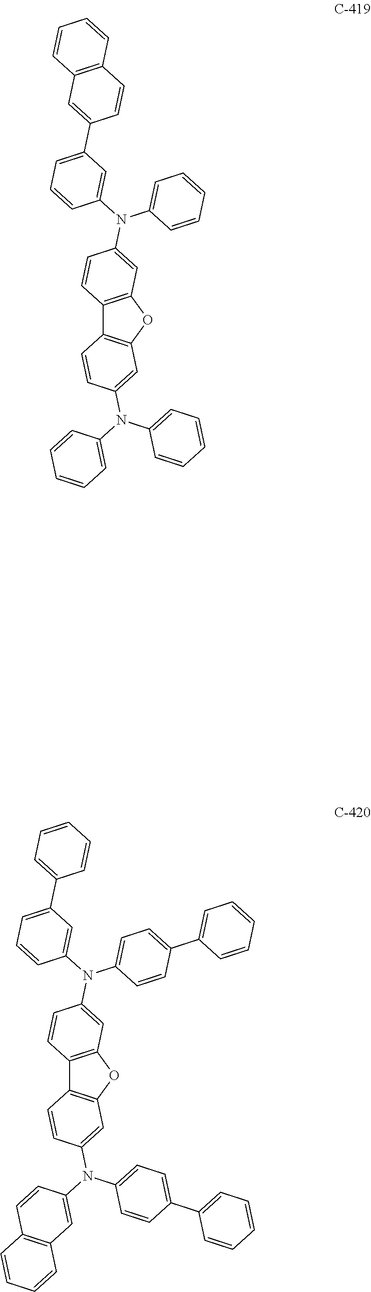 [SMILES: c1ccc(N(c2ccccc2)c2ccc3c(c2)oc2cc(N(c4ccccc4)c4cccc(-c5ccc6ccccc6c5)c4)ccc23)cc1 ; c1ccc(-c2ccc(N(c3cccc(-c4ccccc4)c3)c3ccc4c(c3)oc3cc(N(c5ccc(-c6ccccc6)cc5)c5ccc6ccccc6c5)ccc34)cc2)cc1] 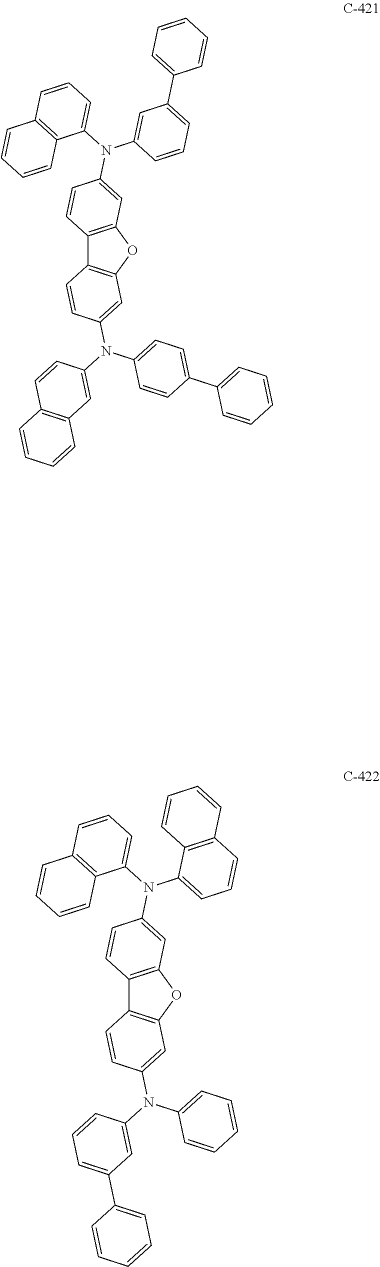 [SMILES: c1ccc(-c2ccc(N(c3ccc4ccccc4c3)c3ccc4c(c3)oc3cc(N(c5cccc(-c6ccccc6)c5)c5cccc6ccccc56)ccc34)cc2)cc1 ; c1ccc(-c2cccc(N(c3ccccc3)c3ccc4c(c3)oc3cc(N(c5cccc6ccccc56)c5cccc6ccccc56)ccc34)c2)cc1] 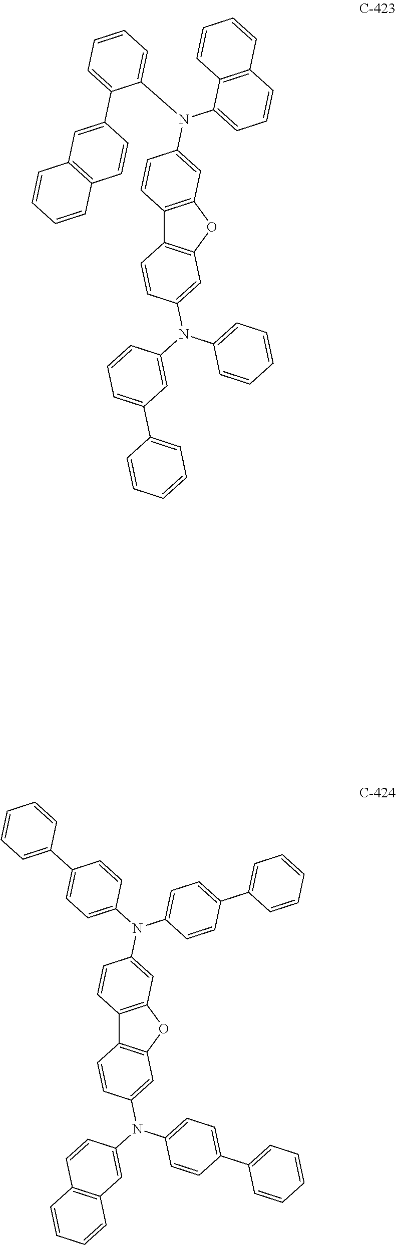 [SMILES: c1ccc(-c2cccc(N(c3ccccc3)c3ccc4c(c3)oc3cc(N(c5ccccc5-c5ccc6ccccc6c5)c5cccc6ccccc56)ccc34)c2)cc1 ; c1ccc(-c2ccc(N(c3ccc(-c4ccccc4)cc3)c3ccc4c(c3)oc3cc(N(c5ccc(-c6ccccc6)cc5)c5ccc6ccccc6c5)ccc34)cc2)cc1] 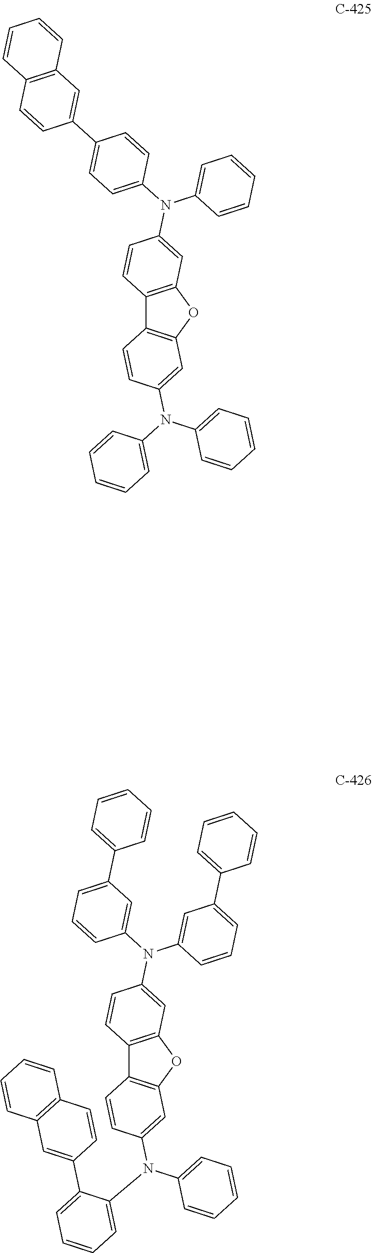 [SMILES: c1ccc(N(c2ccccc2)c2ccc3c(c2)oc2cc(N(c4ccccc4)c4ccc(-c5ccc6ccccc6c5)cc4)ccc23)cc1 ; c1ccc(-c2cccc(N(c3cccc(-c4ccccc4)c3)c3ccc4c(c3)oc3cc(N(c5ccccc5)c5ccccc5-c5ccc6ccccc6c5)ccc34)c2)cc1] 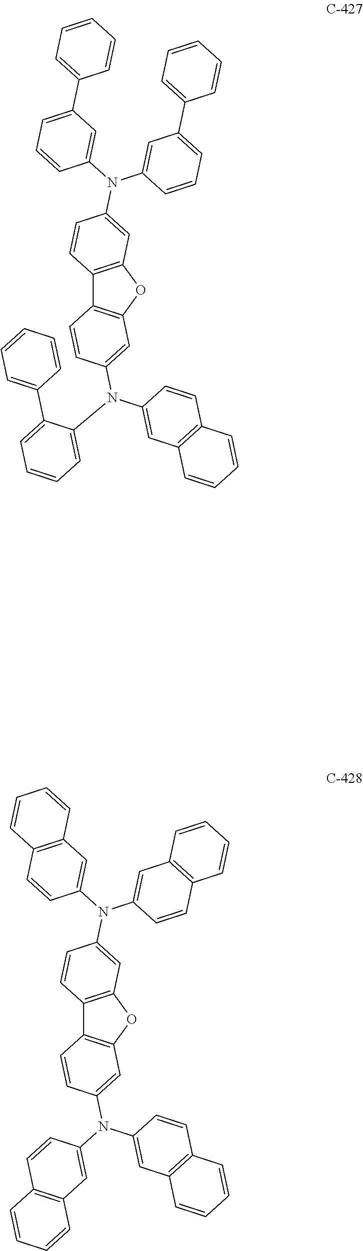 [SMILES: c1ccc(-c2cccc(N(c3cccc(-c4ccccc4)c3)c3ccc4c(c3)oc3cc(N(c5ccc6ccccc6c5)c5ccccc5-c5ccccc5)ccc34)c2)cc1 ; c1ccc2cc(N(c3ccc4ccccc4c3)c3ccc4c(c3)oc3cc(N(c5ccc6ccccc6c5)c5ccc6ccccc6c5)ccc34)ccc2c1] 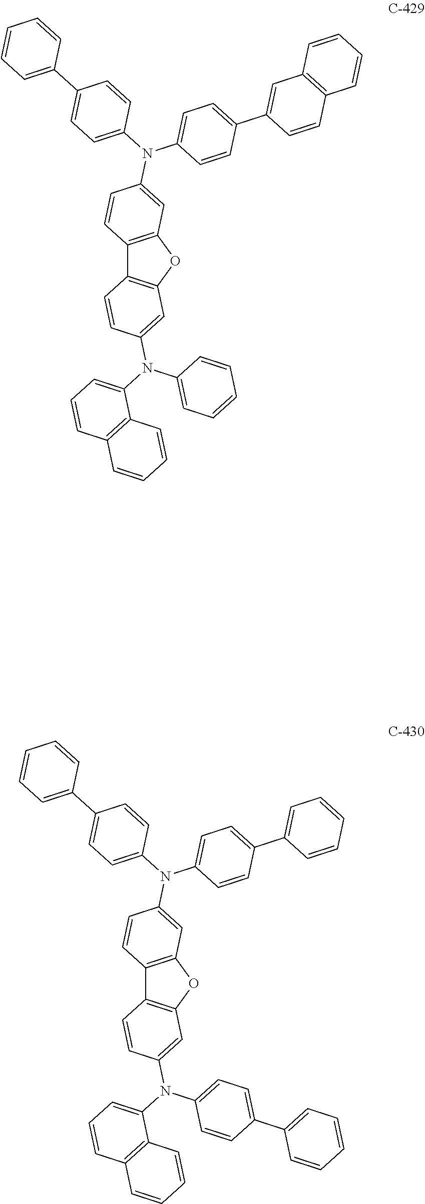 [SMILES: c1ccc(-c2ccc(N(c3ccc(-c4ccc5ccccc5c4)cc3)c3ccc4c(c3)oc3cc(N(c5ccccc5)c5cccc6ccccc56)ccc34)cc2)cc1 ; c1ccc(-c2ccc(N(c3ccc(-c4ccccc4)cc3)c3ccc4c(c3)oc3cc(N(c5ccc(-c6ccccc6)cc5)c5cccc6ccccc56)ccc34)cc2)cc1] 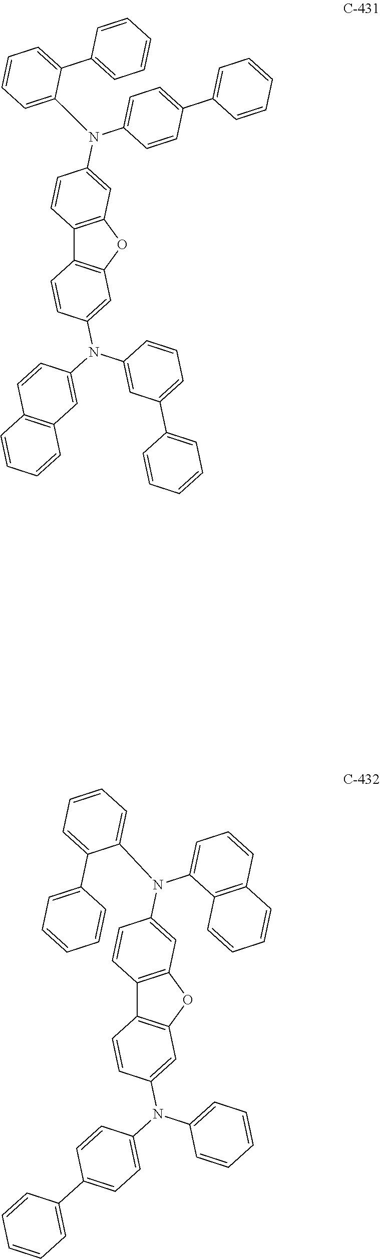 [SMILES: c1ccc(-c2ccc(N(c3ccc4c(c3)oc3cc(N(c5cccc(-c6ccccc6)c5)c5ccc6ccccc6c5)ccc34)c3ccccc3-c3ccccc3)cc2)cc1 ; c1ccc(-c2ccc(N(c3ccccc3)c3ccc4c(c3)oc3cc(N(c5ccccc5-c5ccccc5)c5cccc6ccccc56)ccc34)cc2)cc1] 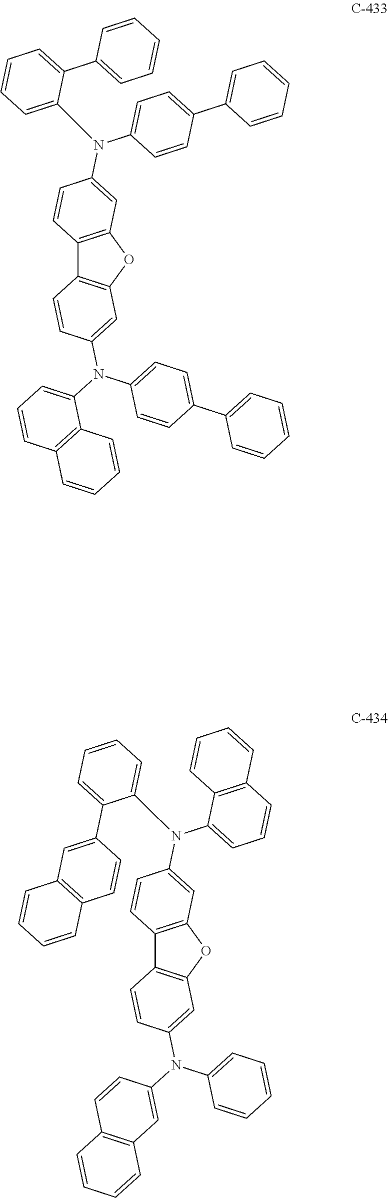 [SMILES: c1ccc(-c2ccc(N(c3ccc4c(c3)oc3cc(N(c5ccc(-c6ccccc6)cc5)c5cccc6ccccc56)ccc34)c3ccccc3-c3ccccc3)cc2)cc1 ; c1ccc(N(c2ccc3ccccc3c2)c2ccc3c(c2)oc2cc(N(c4ccccc4-c4ccc5ccccc5c4)c4cccc5ccccc45)ccc23)cc1] 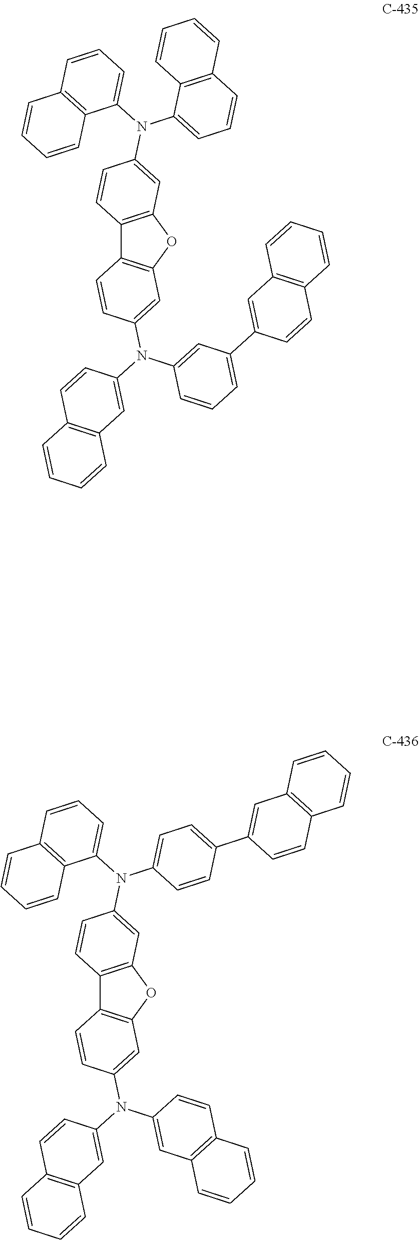 [SMILES: c1cc(-c2ccc3ccccc3c2)cc(N(c2ccc3ccccc3c2)c2ccc3c(c2)oc2cc(N(c4cccc5ccccc45)c4cccc5ccccc45)ccc23)c1 ; c1ccc2cc(-c3ccc(N(c4ccc5c(c4)oc4cc(N(c6ccc7ccccc7c6)c6ccc7ccccc7c6)ccc45)c4cccc5ccccc45)cc3)ccc2c1] 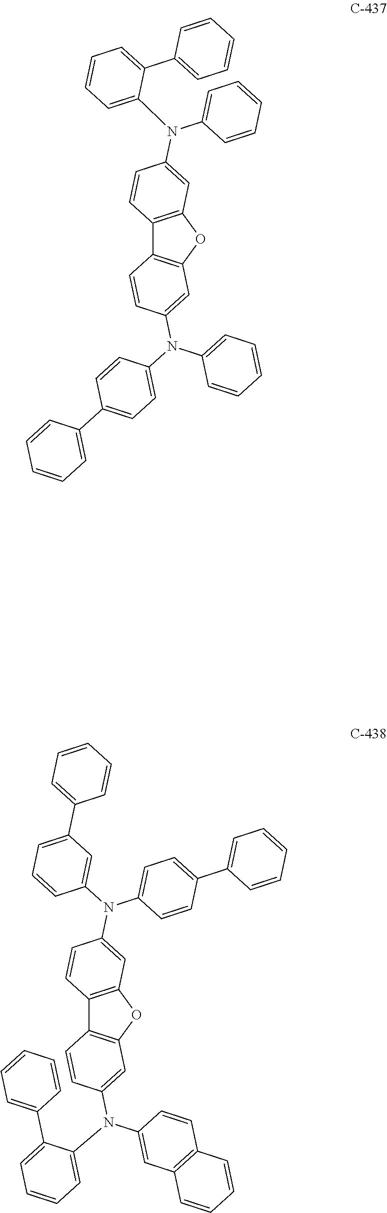 [SMILES: c1ccc(-c2ccc(N(c3ccccc3)c3ccc4c(c3)oc3cc(N(c5ccccc5)c5ccccc5-c5ccccc5)ccc34)cc2)cc1 ; c1ccc(-c2ccc(N(c3cccc(-c4ccccc4)c3)c3ccc4c(c3)oc3cc(N(c5ccc6ccccc6c5)c5ccccc5-c5ccccc5)ccc34)cc2)cc1] 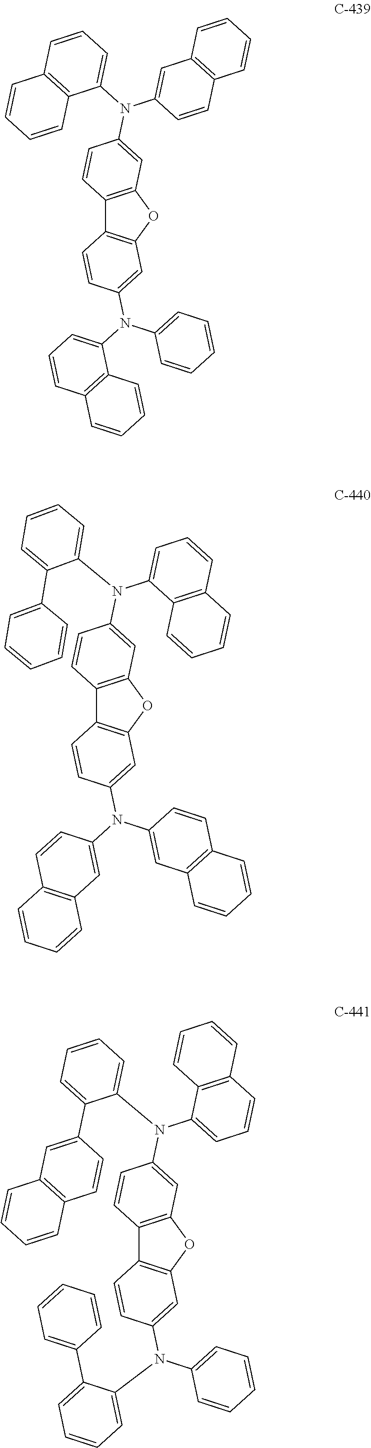 [SMILES: c1ccc(N(c2ccc3c(c2)oc2cc(N(c4ccc5ccccc5c4)c4cccc5ccccc45)ccc23)c2cccc3ccccc23)cc1 ; c1ccc(-c2ccccc2N(c2ccc3c(c2)oc2cc(N(c4ccc5ccccc5c4)c4ccc5ccccc5c4)ccc23)c2cccc3ccccc23)cc1 ; c1ccc(-c2ccccc2N(c2ccccc2)c2ccc3c(c2)oc2cc(N(c4ccccc4-c4ccc5ccccc5c4)c4cccc5ccccc45)ccc23)cc1] 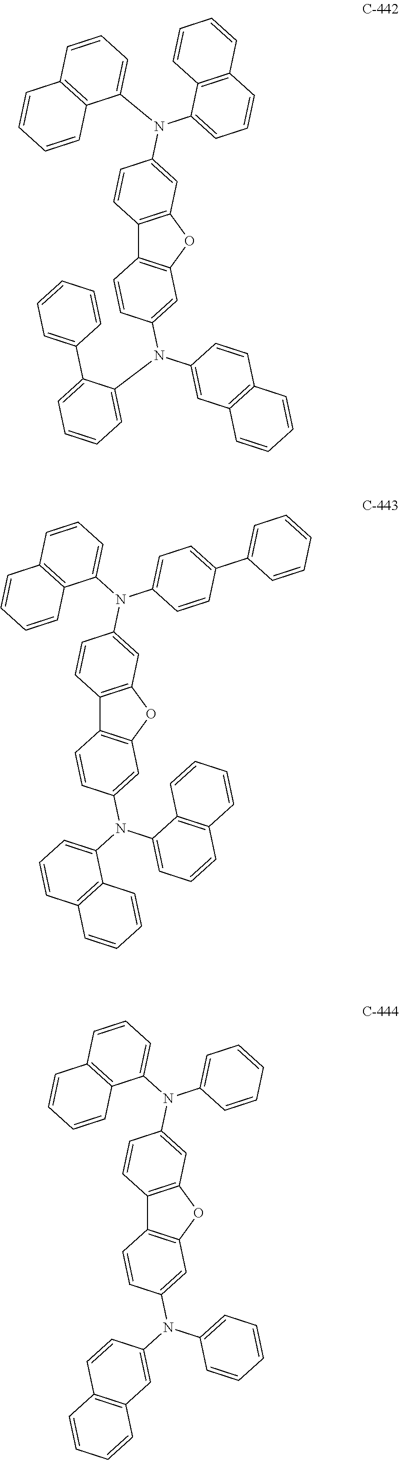 [SMILES: c1ccc(-c2ccccc2N(c2ccc3ccccc3c2)c2ccc3c(c2)oc2cc(N(c4cccc5ccccc45)c4cccc5ccccc45)ccc23)cc1 ; c1ccc(-c2ccc(N(c3ccc4c(c3)oc3cc(N(c5cccc6ccccc56)c5cccc6ccccc56)ccc34)c3cccc4ccccc34)cc2)cc1 ; c1ccc(N(c2ccc3ccccc3c2)c2ccc3c(c2)oc2cc(N(c4ccccc4)c4cccc5ccccc45)ccc23)cc1] 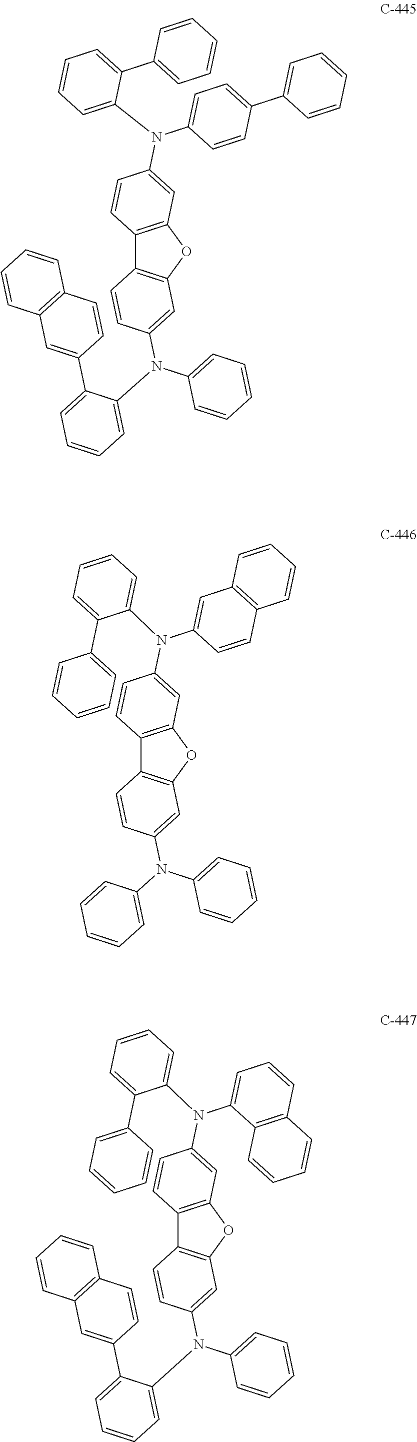 [SMILES: c1ccc(-c2ccc(N(c3ccc4c(c3)oc3cc(N(c5ccccc5)c5ccccc5-c5ccc6ccccc6c5)ccc34)c3ccccc3-c3ccccc3)cc2)cc1 ; c1ccc(-c2ccccc2N(c2ccc3ccccc3c2)c2ccc3c(c2)oc2cc(N(c4ccccc4)c4ccccc4)ccc23)cc1 ; c1ccc(-c2ccccc2N(c2ccc3c(c2)oc2cc(N(c4ccccc4)c4ccccc4-c4ccc5ccccc5c4)ccc23)c2cccc3ccccc23)cc1] 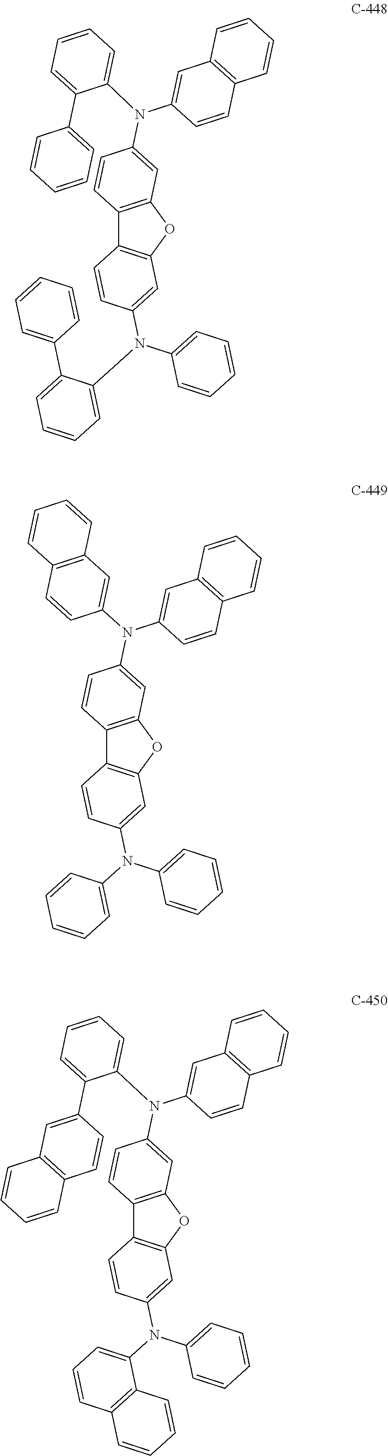 [SMILES: c1ccc(-c2ccccc2N(c2ccccc2)c2ccc3c(c2)oc2cc(N(c4ccc5ccccc5c4)c4ccccc4-c4ccccc4)ccc23)cc1 ; c1ccc(N(c2ccccc2)c2ccc3c(c2)oc2cc(N(c4ccc5ccccc5c4)c4ccc5ccccc5c4)ccc23)cc1 ; c1ccc(N(c2ccc3c(c2)oc2cc(N(c4ccc5ccccc5c4)c4ccccc4-c4ccc5ccccc5c4)ccc23)c2cccc3ccccc23)cc1] 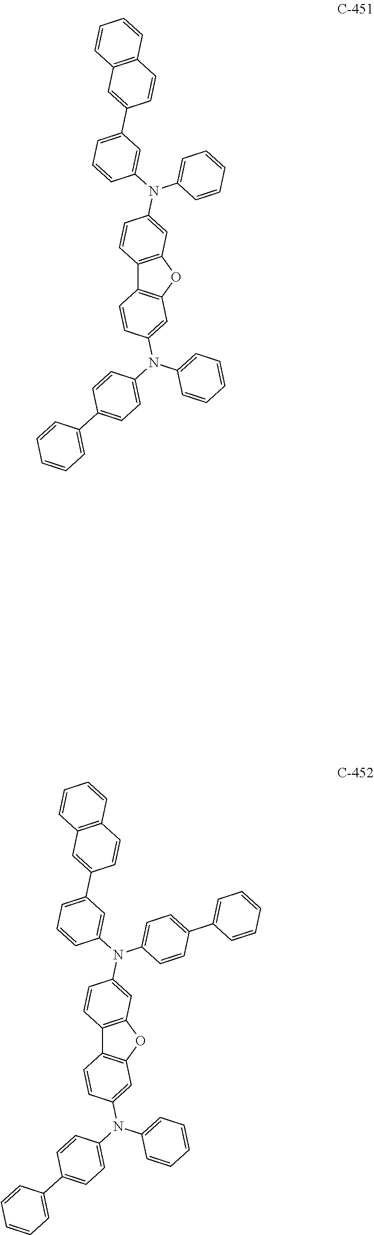 [SMILES: c1ccc(-c2ccc(N(c3ccccc3)c3ccc4c(c3)oc3cc(N(c5ccccc5)c5cccc(-c6ccc7ccccc7c6)c5)ccc34)cc2)cc1 ; c1ccc(-c2ccc(N(c3ccccc3)c3ccc4c(c3)oc3cc(N(c5ccc(-c6ccccc6)cc5)c5cccc(-c6ccc7ccccc7c6)c5)ccc34)cc2)cc1] 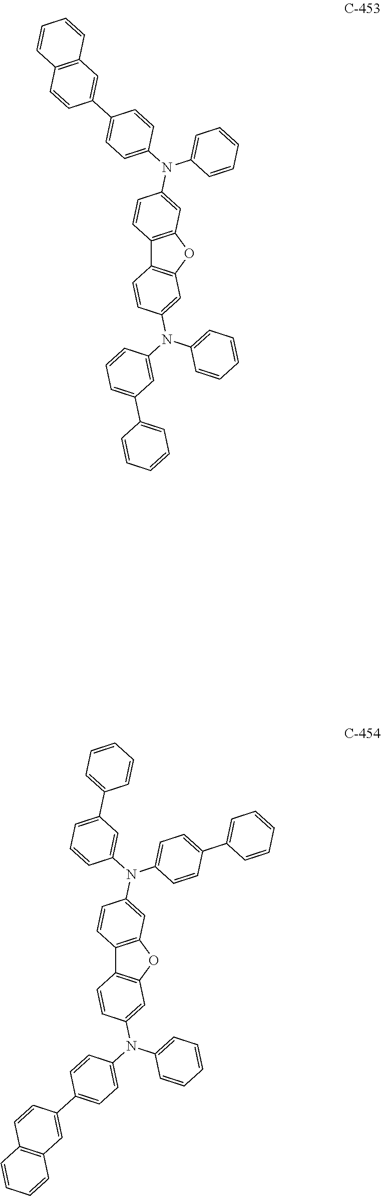 [SMILES: c1ccc(-c2cccc(N(c3ccccc3)c3ccc4c(c3)oc3cc(N(c5ccccc5)c5ccc(-c6ccc7ccccc7c6)cc5)ccc34)c2)cc1 ; c1ccc(-c2ccc(N(c3cccc(-c4ccccc4)c3)c3ccc4c(c3)oc3cc(N(c5ccccc5)c5ccc(-c6ccc7ccccc7c6)cc5)ccc34)cc2)cc1] 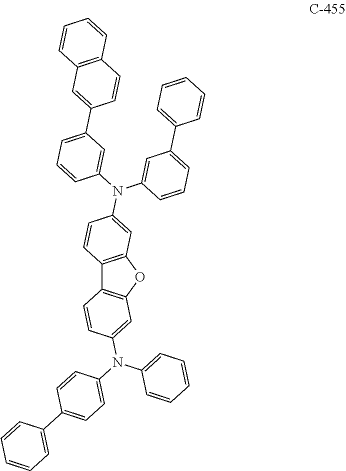 [SMILES: c1ccc(-c2ccc(N(c3ccccc3)c3ccc4c(c3)oc3cc(N(c5cccc(-c6ccccc6)c5)c5cccc(-c6ccc7ccccc7c6)c5)ccc34)cc2)cc1] 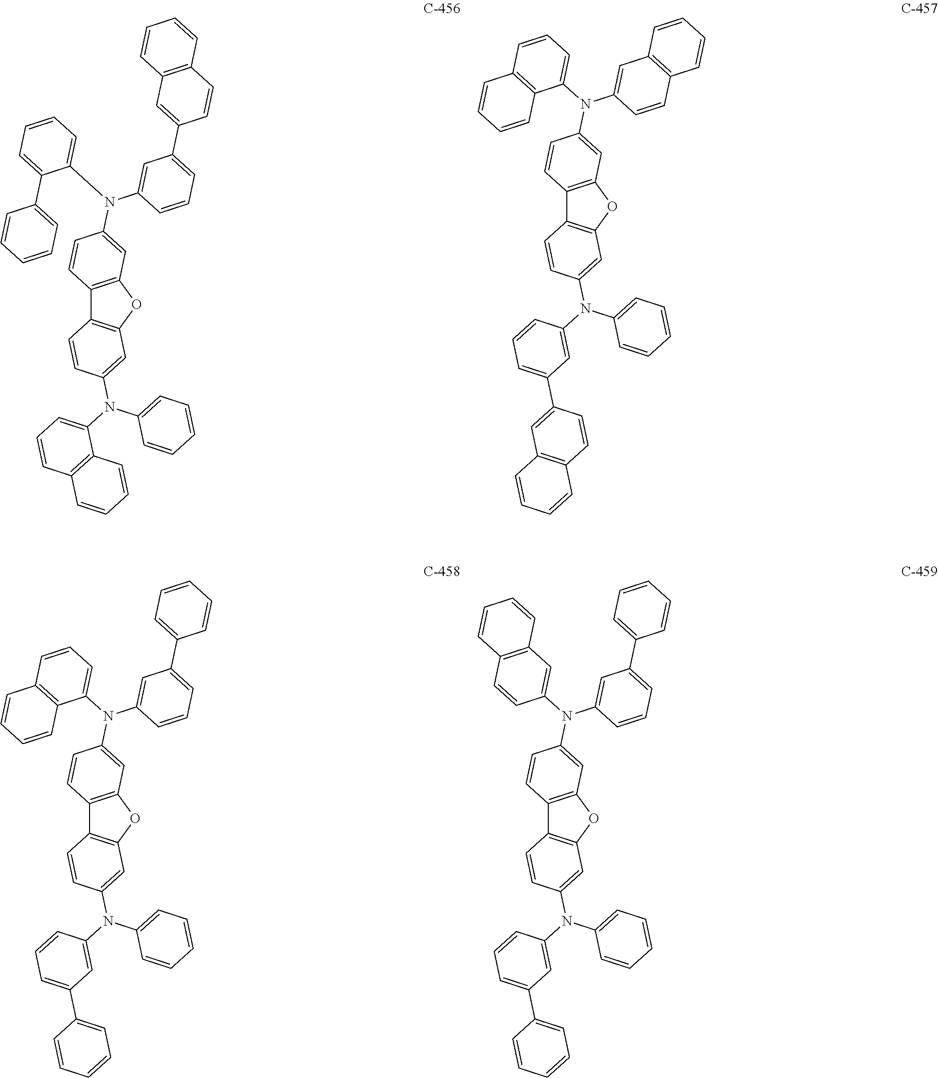 [SMILES: c1ccc(-c2ccccc2N(c2cccc(-c3ccc4ccccc4c3)c2)c2ccc3c(c2)oc2cc(N(c4ccccc4)c4cccc5ccccc45)ccc23)cc1 ; c1ccc(N(c2cccc(-c3ccc4ccccc4c3)c2)c2ccc3c(c2)oc2cc(N(c4ccc5ccccc5c4)c4cccc5ccccc45)ccc23)cc1 ; c1ccc(-c2cccc(N(c3ccccc3)c3ccc4c(c3)oc3cc(N(c5cccc(-c6ccccc6)c5)c5cccc6ccccc56)ccc34)c2)cc1 ; c1ccc(-c2cccc(N(c3ccccc3)c3ccc4c(c3)oc3cc(N(c5cccc(-c6ccccc6)c5)c5ccc6ccccc6c5)ccc34)c2)cc1] 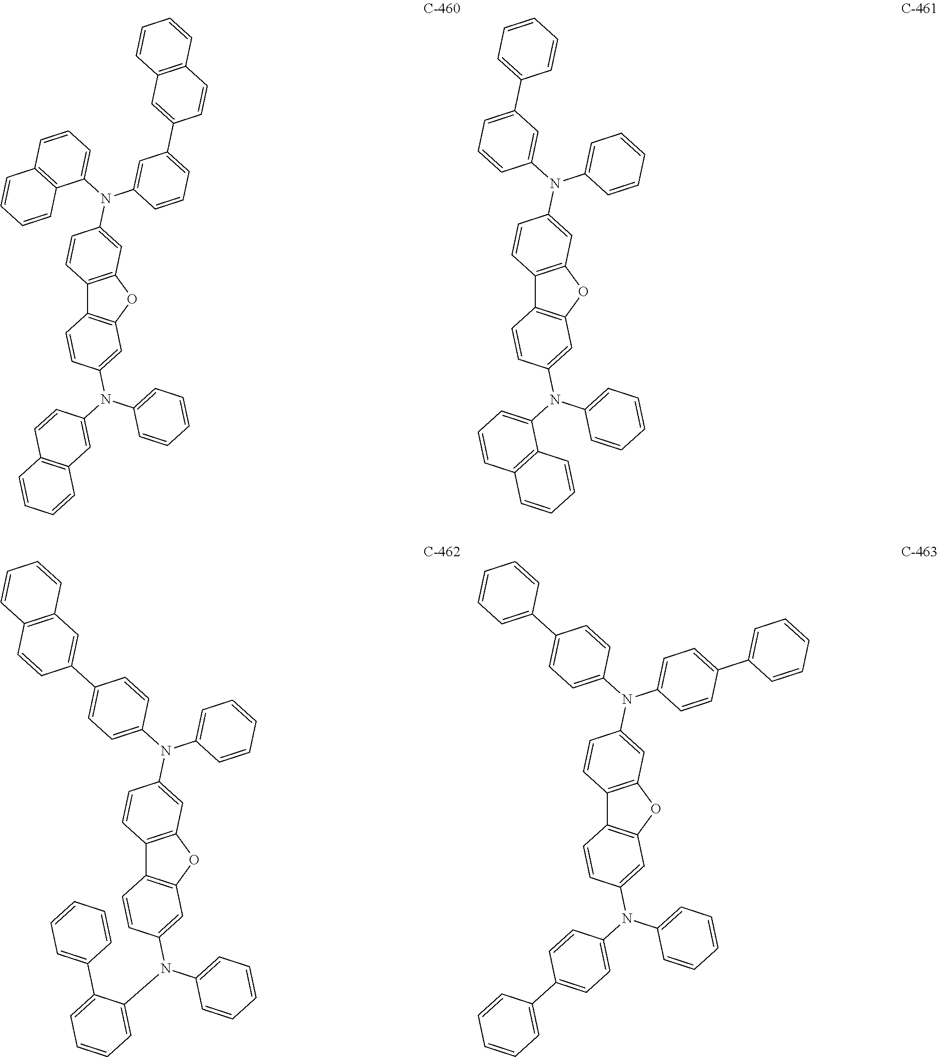 [SMILES: c1ccc(N(c2ccc3ccccc3c2)c2ccc3c(c2)oc2cc(N(c4cccc(-c5ccc6ccccc6c5)c4)c4cccc5ccccc45)ccc23)cc1 ; c1ccc(-c2cccc(N(c3ccccc3)c3ccc4c(c3)oc3cc(N(c5ccccc5)c5cccc6ccccc56)ccc34)c2)cc1 ; c1ccc(-c2ccccc2N(c2ccccc2)c2ccc3c(c2)oc2cc(N(c4ccccc4)c4ccc(-c5ccc6ccccc6c5)cc4)ccc23)cc1 ; c1ccc(-c2ccc(N(c3ccccc3)c3ccc4c(c3)oc3cc(N(c5ccc(-c6ccccc6)cc5)c5ccc(-c6ccccc6)cc5)ccc34)cc2)cc1] 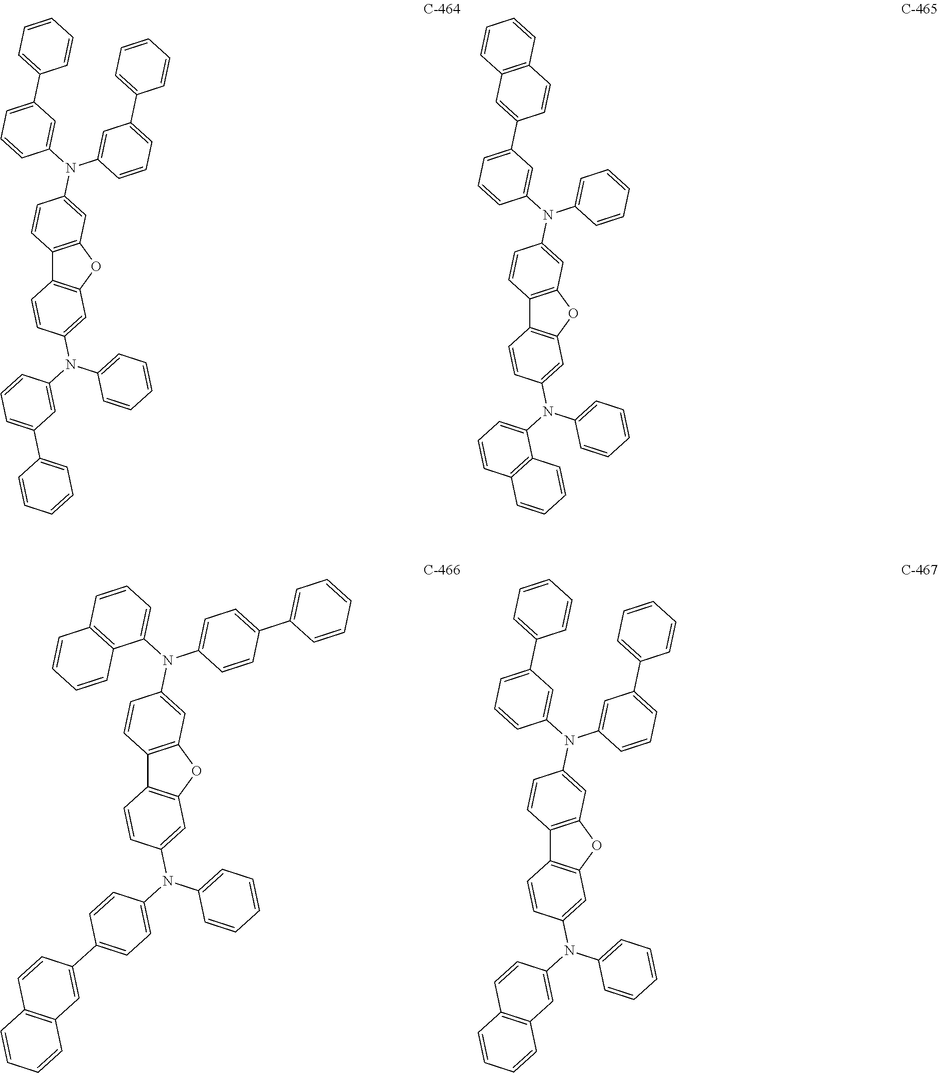 [SMILES: c1ccc(-c2cccc(N(c3ccccc3)c3ccc4c(c3)oc3cc(N(c5cccc(-c6ccccc6)c5)c5cccc(-c6ccccc6)c5)ccc34)c2)cc1 ; c1ccc(N(c2cccc(-c3ccc4ccccc4c3)c2)c2ccc3c(c2)oc2cc(N(c4ccccc4)c4cccc5ccccc45)ccc23)cc1 ; c1ccc(-c2ccc(N(c3ccc4c(c3)oc3cc(N(c5ccccc5)c5ccc(-c6ccc7ccccc7c6)cc5)ccc34)c3cccc4ccccc34)cc2)cc1 ; c1ccc(-c2cccc(N(c3cccc(-c4ccccc4)c3)c3ccc4c(c3)oc3cc(N(c5ccccc5)c5ccc6ccccc6c5)ccc34)c2)cc1] 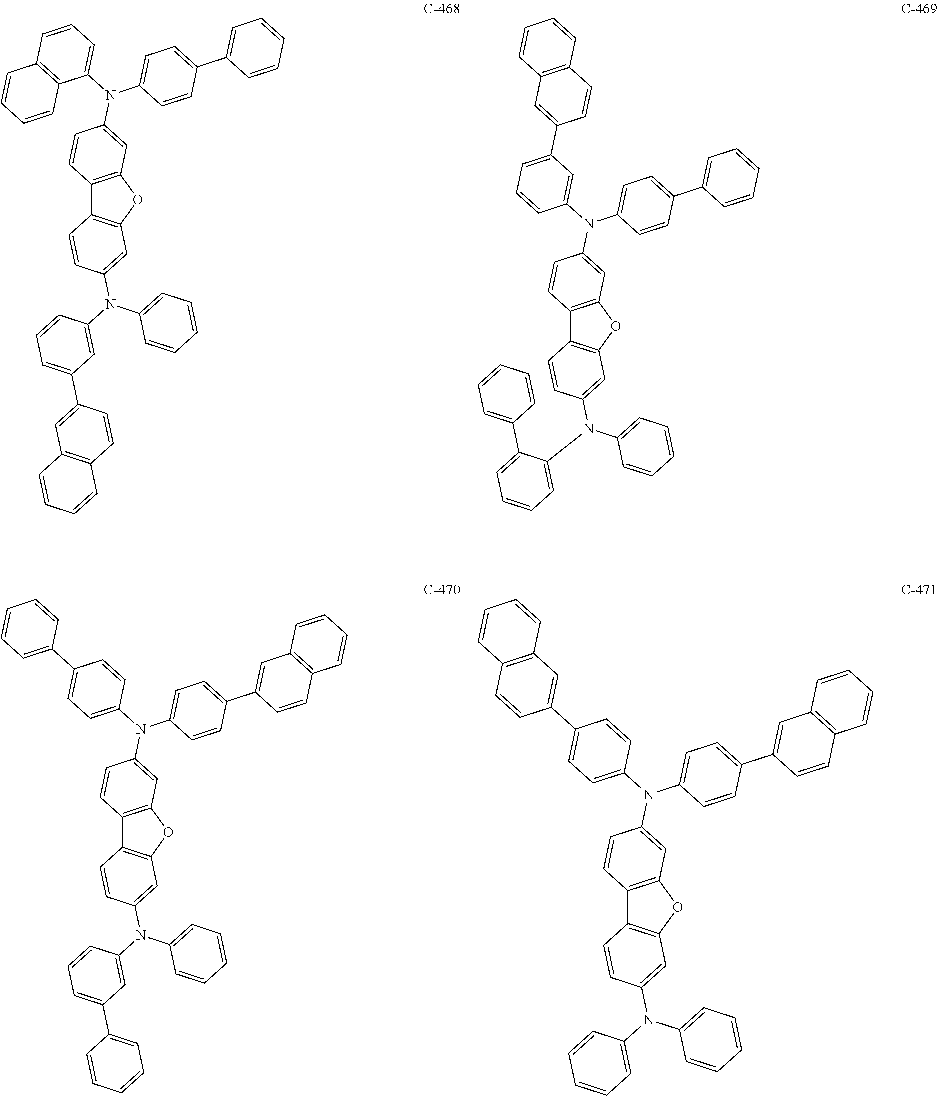 [SMILES: c1ccc(-c2ccc(N(c3ccc4c(c3)oc3cc(N(c5ccccc5)c5cccc(-c6ccc7ccccc7c6)c5)ccc34)c3cccc4ccccc34)cc2)cc1 ; c1ccc(-c2ccc(N(c3cccc(-c4ccc5ccccc5c4)c3)c3ccc4c(c3)oc3cc(N(c5ccccc5)c5ccccc5-c5ccccc5)ccc34)cc2)cc1 ; c1ccc(-c2ccc(N(c3ccc(-c4ccc5ccccc5c4)cc3)c3ccc4c(c3)oc3cc(N(c5ccccc5)c5cccc(-c6ccccc6)c5)ccc34)cc2)cc1 ; c1ccc(N(c2ccccc2)c2ccc3c(c2)oc2cc(N(c4ccc(-c5ccc6ccccc6c5)cc4)c4ccc(-c5ccc6ccccc6c5)cc4)ccc23)cc1] 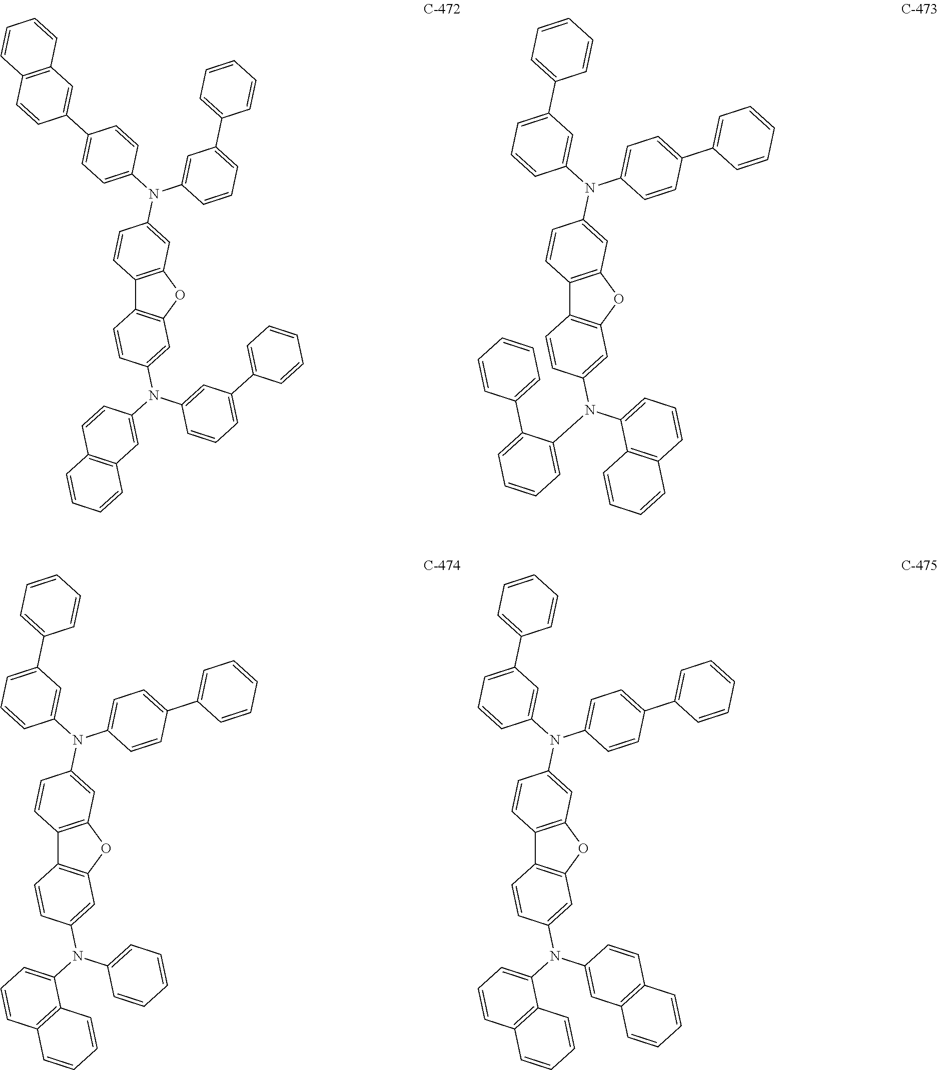 [SMILES: c1ccc(-c2cccc(N(c3ccc(-c4ccc5ccccc5c4)cc3)c3ccc4c(c3)oc3cc(N(c5cccc(-c6ccccc6)c5)c5ccc6ccccc6c5)ccc34)c2)cc1 ; c1ccc(-c2ccc(N(c3cccc(-c4ccccc4)c3)c3ccc4c(c3)oc3cc(N(c5ccccc5-c5ccccc5)c5cccc6ccccc56)ccc34)cc2)cc1 ; c1ccc(-c2ccc(N(c3cccc(-c4ccccc4)c3)c3ccc4c(c3)oc3cc(N(c5ccccc5)c5cccc6ccccc56)ccc34)cc2)cc1 ; c1ccc(-c2ccc(N(c3cccc(-c4ccccc4)c3)c3ccc4c(c3)oc3cc(N(c5ccc6ccccc6c5)c5cccc6ccccc56)ccc34)cc2)cc1] 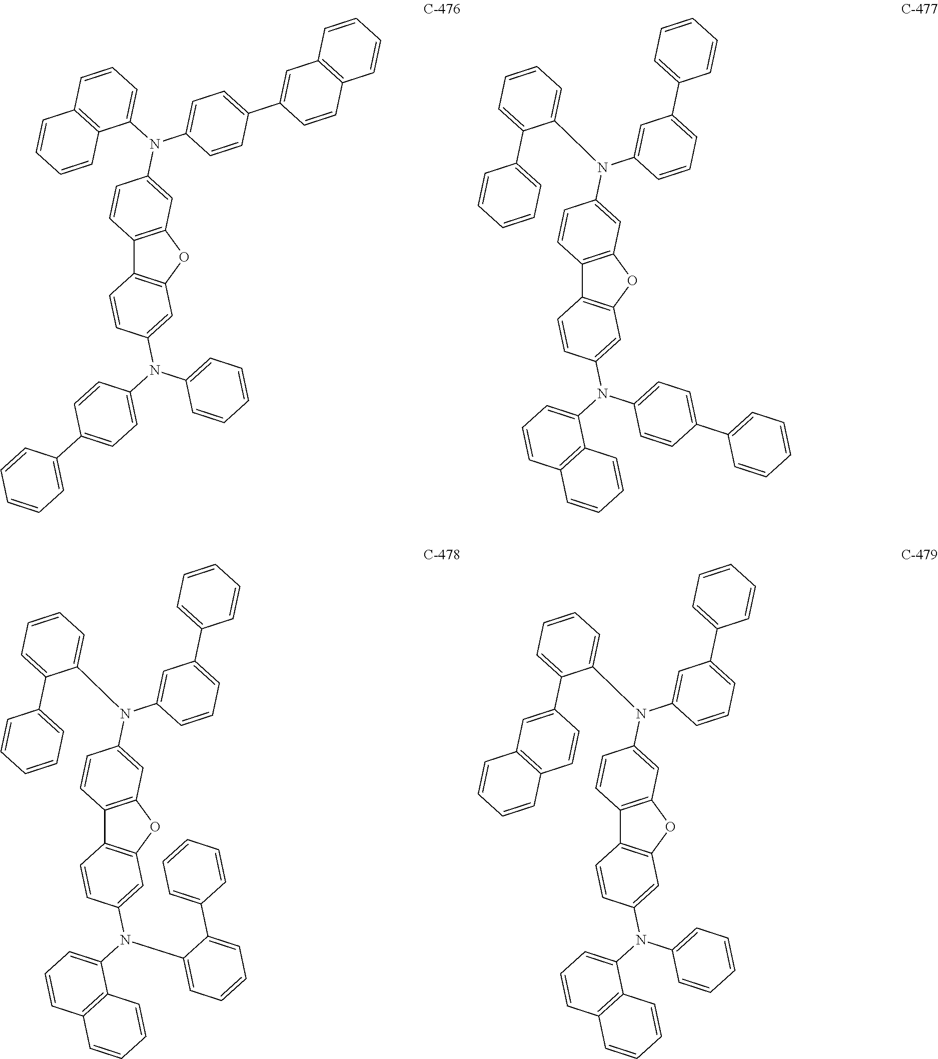 [SMILES: c1ccc(-c2ccc(N(c3ccccc3)c3ccc4c(c3)oc3cc(N(c5ccc(-c6ccc7ccccc7c6)cc5)c5cccc6ccccc56)ccc34)cc2)cc1 ; c1ccc(-c2ccc(N(c3ccc4c(c3)oc3cc(N(c5cccc(-c6ccccc6)c5)c5ccccc5-c5ccccc5)ccc34)c3cccc4ccccc34)cc2)cc1 ; c1ccc(-c2cccc(N(c3ccc4c(c3)oc3cc(N(c5ccccc5-c5ccccc5)c5cccc6ccccc56)ccc34)c3ccccc3-c3ccccc3)c2)cc1 ; c1ccc(-c2cccc(N(c3ccc4c(c3)oc3cc(N(c5ccccc5)c5cccc6ccccc56)ccc34)c3ccccc3-c3ccc4ccccc4c3)c2)cc1] 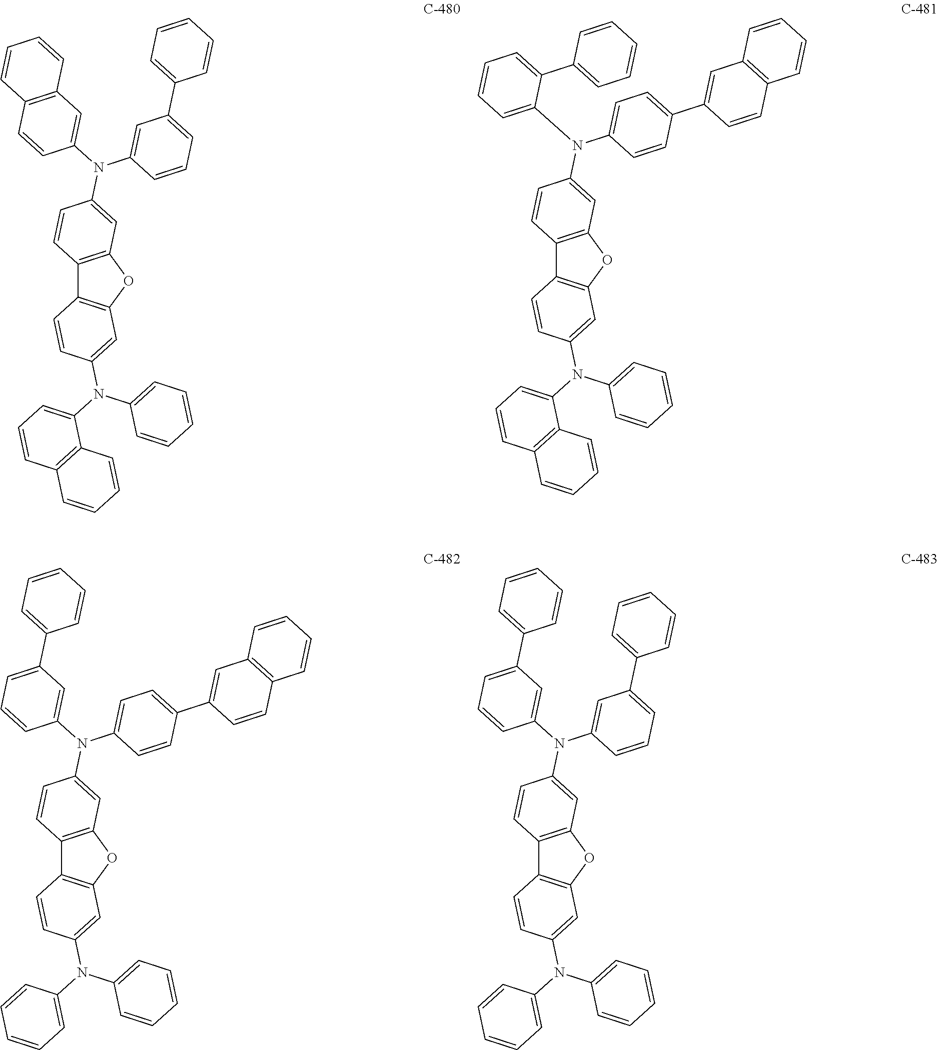 [SMILES: c1ccc(-c2cccc(N(c3ccc4ccccc4c3)c3ccc4c(c3)oc3cc(N(c5ccccc5)c5cccc6ccccc56)ccc34)c2)cc1 ; c1ccc(-c2ccccc2N(c2ccc(-c3ccc4ccccc4c3)cc2)c2ccc3c(c2)oc2cc(N(c4ccccc4)c4cccc5ccccc45)ccc23)cc1 ; c1ccc(-c2cccc(N(c3ccc(-c4ccc5ccccc5c4)cc3)c3ccc4c(c3)oc3cc(N(c5ccccc5)c5ccccc5)ccc34)c2)cc1 ; c1ccc(-c2cccc(N(c3cccc(-c4ccccc4)c3)c3ccc4c(c3)oc3cc(N(c5ccccc5)c5ccccc5)ccc34)c2)cc1] 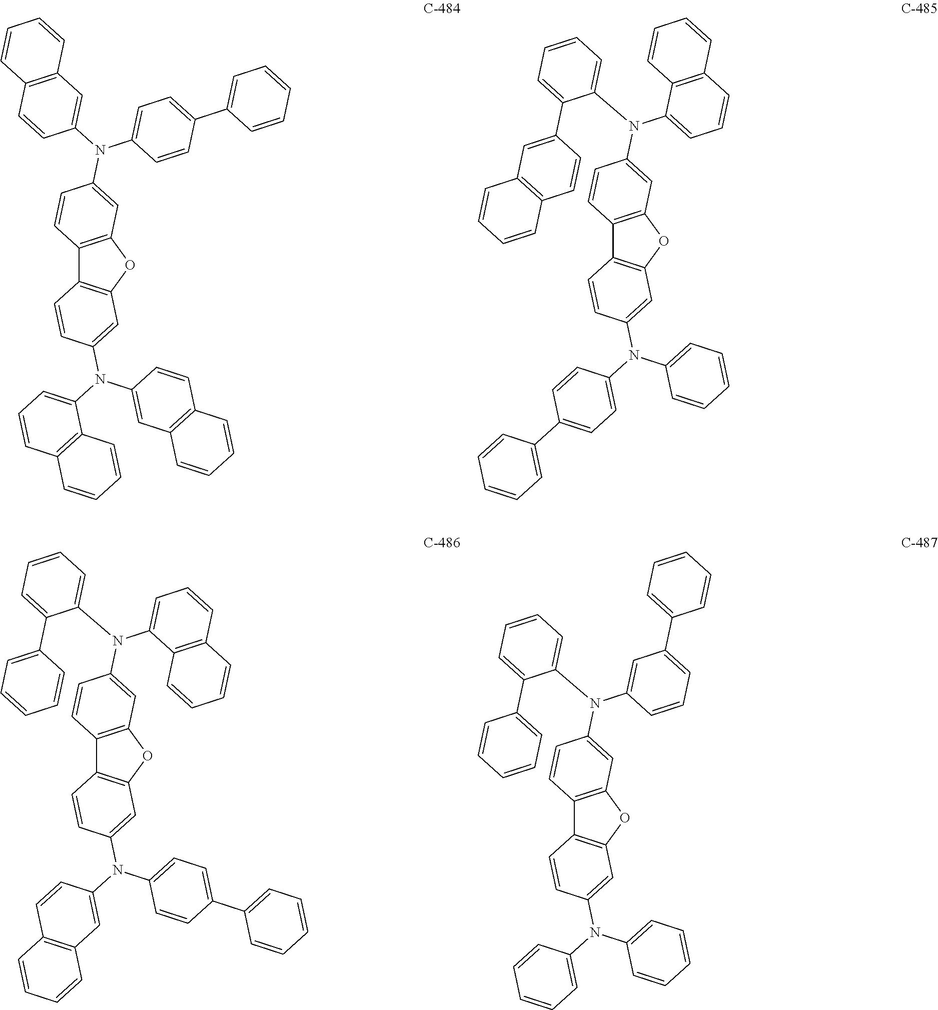 [SMILES: c1ccc(-c2ccc(N(c3ccc4ccccc4c3)c3ccc4c(c3)oc3cc(N(c5ccc6ccccc6c5)c5cccc6ccccc56)ccc34)cc2)cc1 ; c1ccc(-c2ccc(N(c3ccccc3)c3ccc4c(c3)oc3cc(N(c5ccccc5-c5ccc6ccccc6c5)c5cccc6ccccc56)ccc34)cc2)cc1 ; c1ccc(-c2ccc(N(c3ccc4ccccc4c3)c3ccc4c(c3)oc3cc(N(c5ccccc5-c5ccccc5)c5cccc6ccccc56)ccc34)cc2)cc1 ; c1ccc(-c2cccc(N(c3ccc4c(c3)oc3cc(N(c5ccccc5)c5ccccc5)ccc34)c3ccccc3-c3ccccc3)c2)cc1] 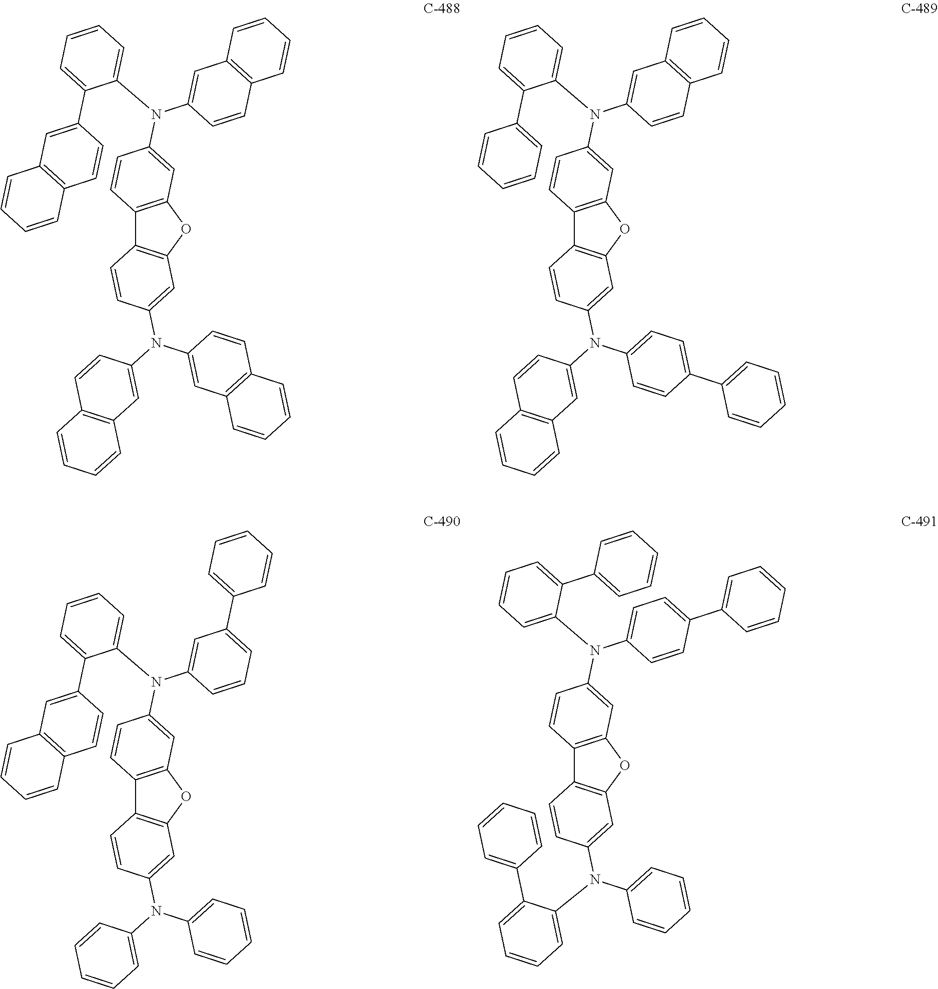 [SMILES: c1ccc(N(c2ccc3ccccc3c2)c2ccc3c(c2)oc2cc(N(c4ccc5ccccc5c4)c4ccc5ccccc5c4)ccc23)c(-c2ccc3ccccc3c2)c1 ; c1ccc(-c2ccc(N(c3ccc4ccccc4c3)c3ccc4c(c3)oc3cc(N(c5ccc6ccccc6c5)c5ccccc5-c5ccccc5)ccc34)cc2)cc1 ; c1ccc(-c2cccc(N(c3ccc4c(c3)oc3cc(N(c5ccccc5)c5ccccc5)ccc34)c3ccccc3-c3ccc4ccccc4c3)c2)cc1 ; c1ccc(-c2ccc(N(c3ccc4c(c3)oc3cc(N(c5ccccc5)c5ccccc5-c5ccccc5)ccc34)c3ccccc3-c3ccccc3)cc2)cc1] 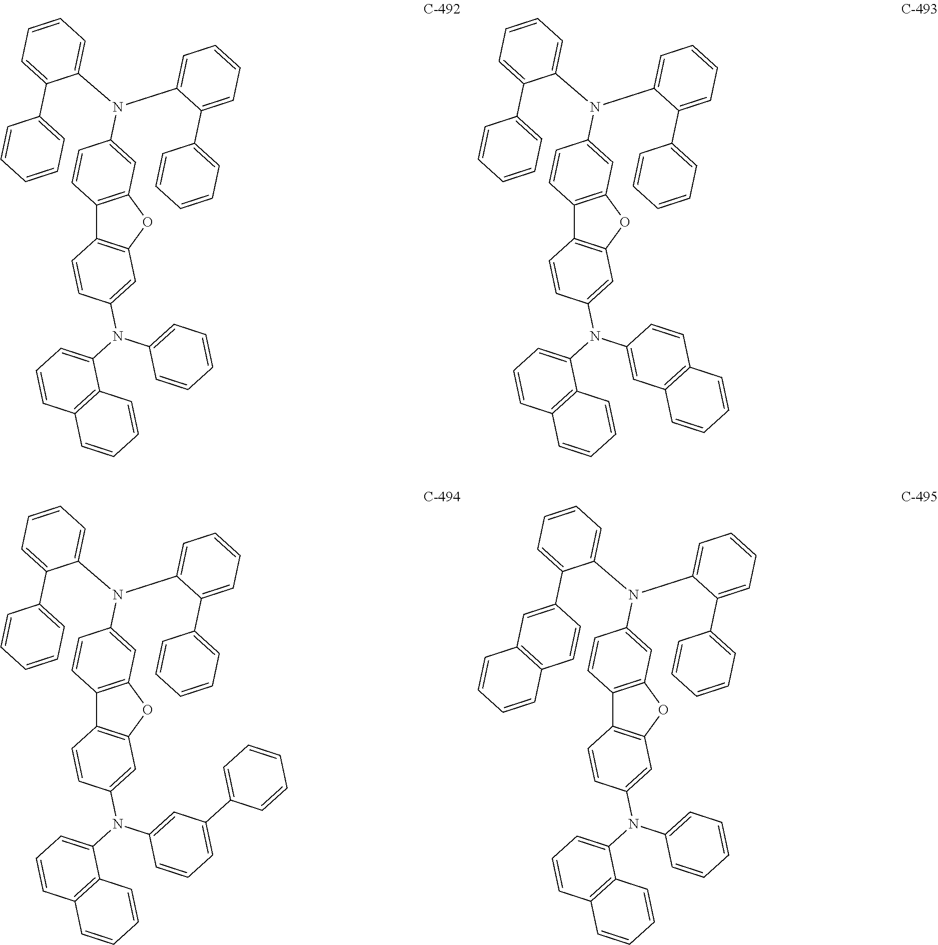 [SMILES: c1ccc(-c2ccccc2N(c2ccc3c(c2)oc2cc(N(c4ccccc4)c4cccc5ccccc45)ccc23)c2ccccc2-c2ccccc2)cc1 ; c1ccc(-c2ccccc2N(c2ccc3c(c2)oc2cc(N(c4ccc5ccccc5c4)c4cccc5ccccc45)ccc23)c2ccccc2-c2ccccc2)cc1 ; c1ccc(-c2cccc(N(c3ccc4c(c3)oc3cc(N(c5ccccc5-c5ccccc5)c5ccccc5-c5ccccc5)ccc34)c3cccc4ccccc34)c2)cc1 ; c1ccc(-c2ccccc2N(c2ccc3c(c2)oc2cc(N(c4ccccc4)c4cccc5ccccc45)ccc23)c2ccccc2-c2ccc3ccccc3c2)cc1] 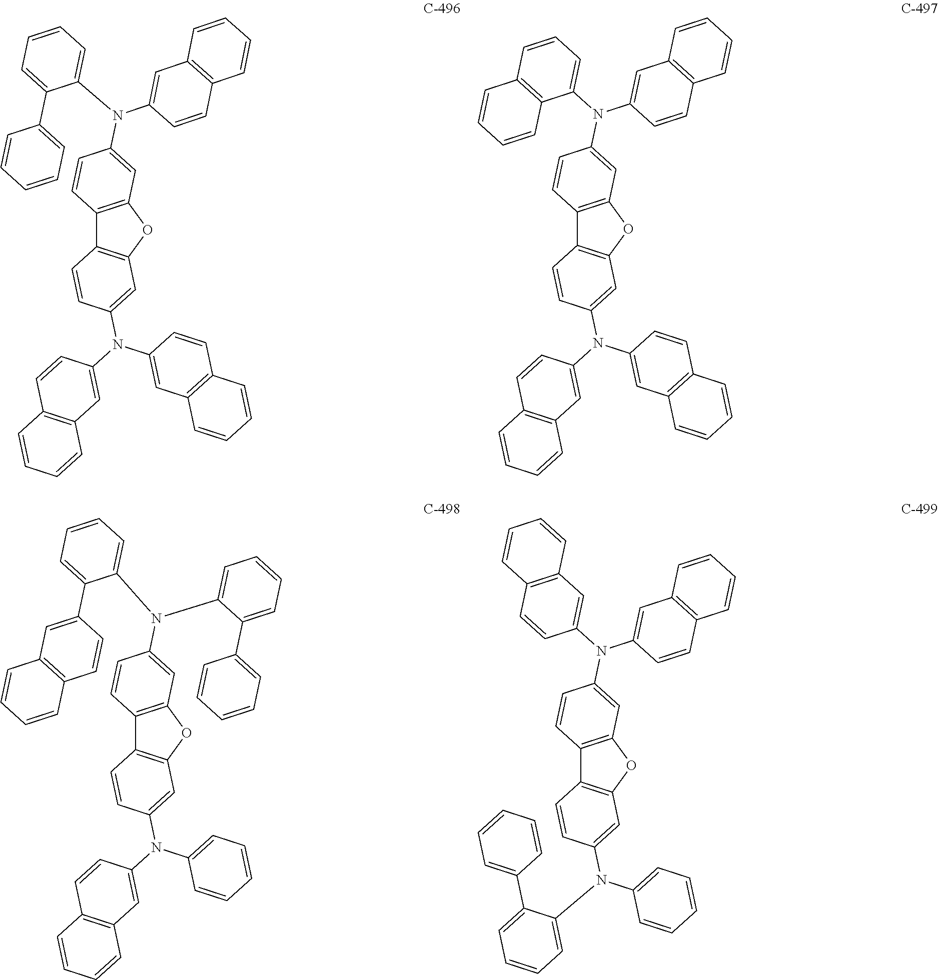 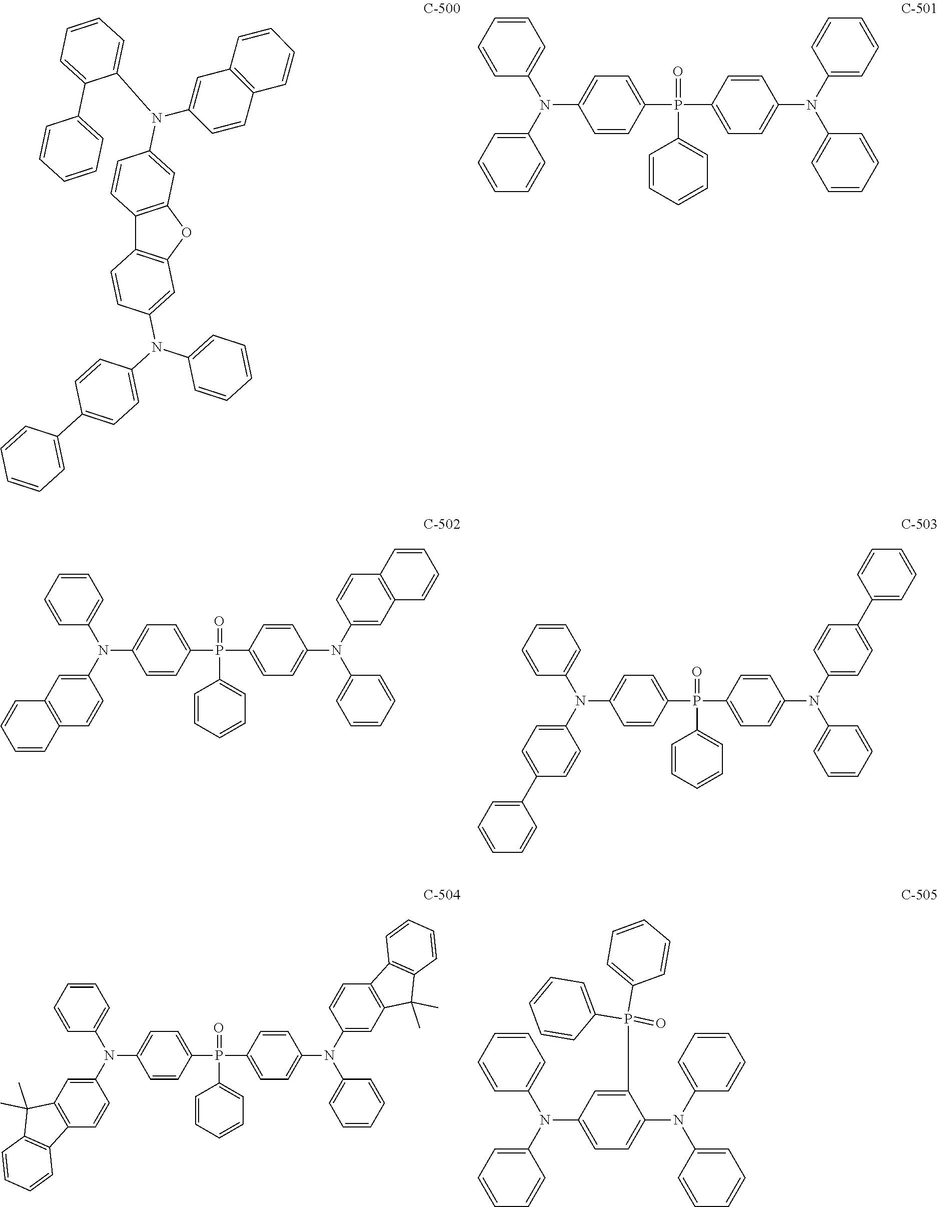 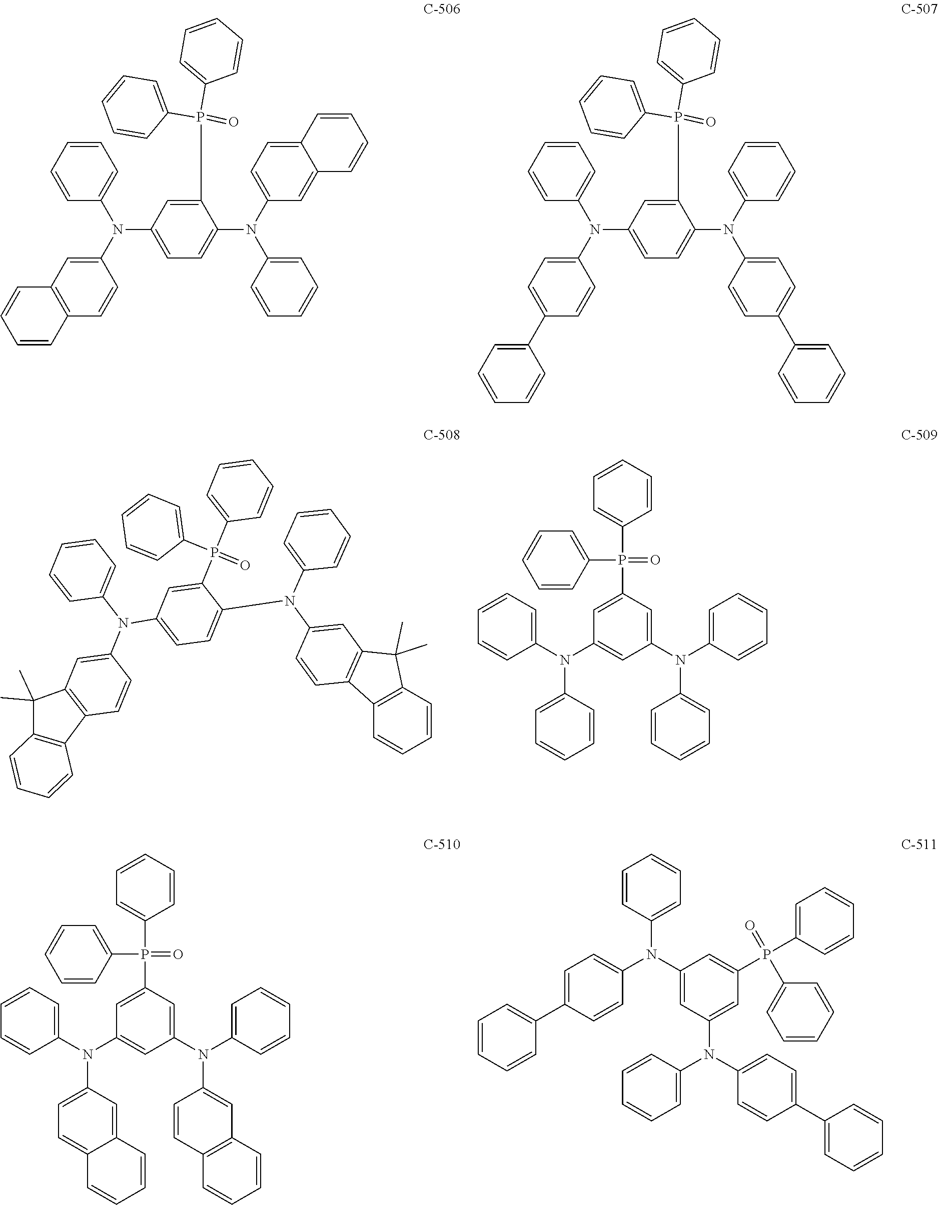 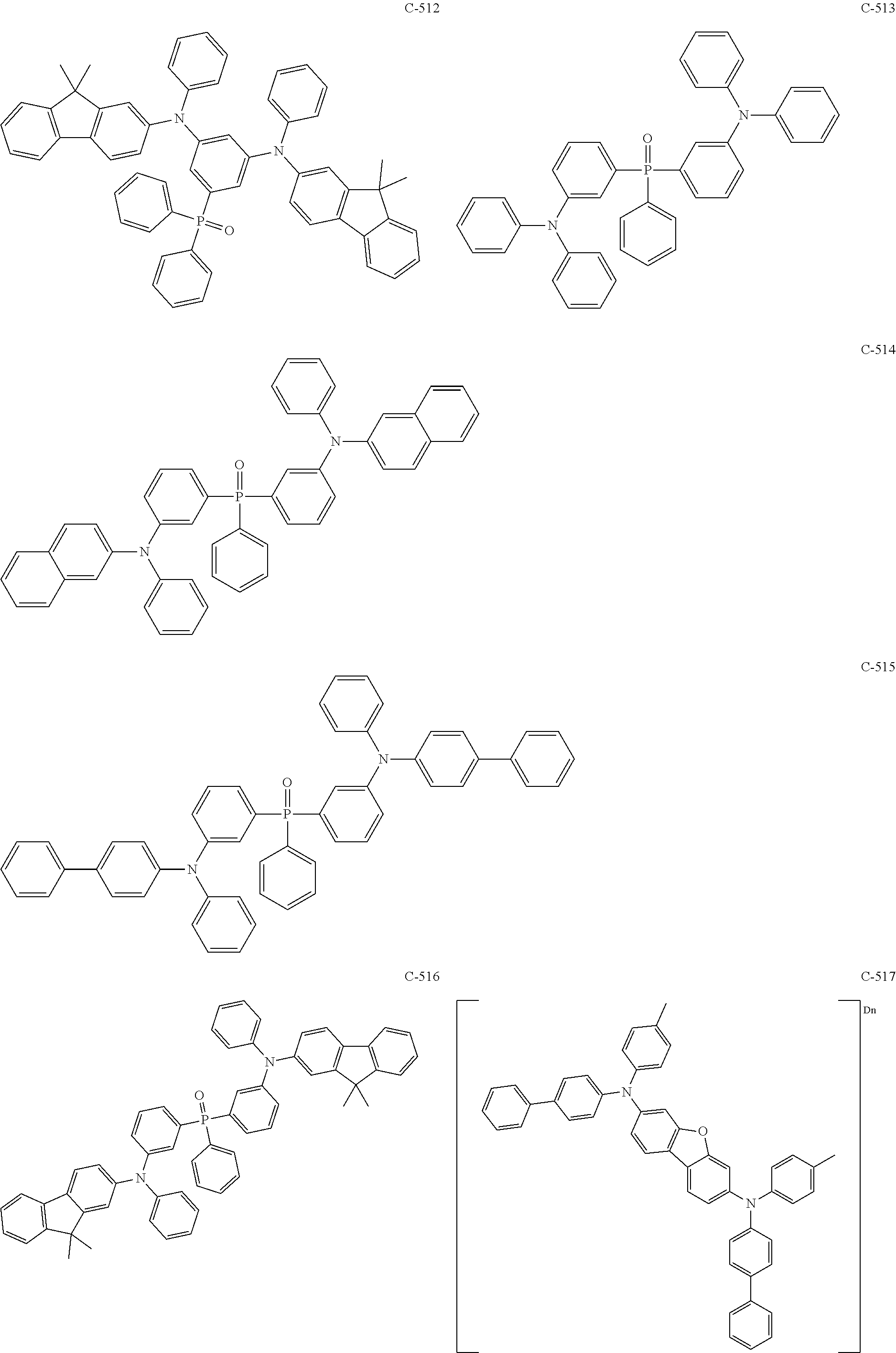 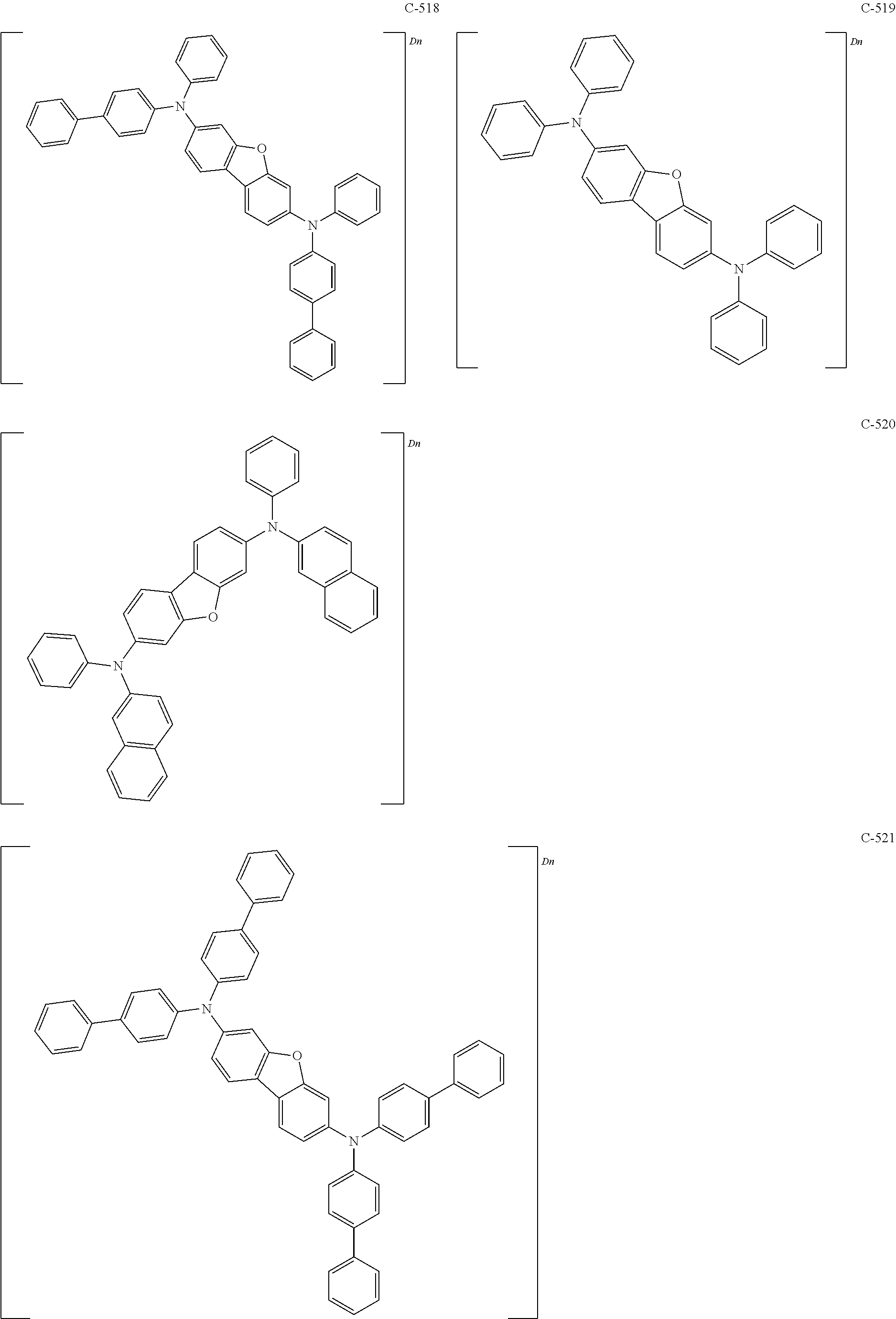 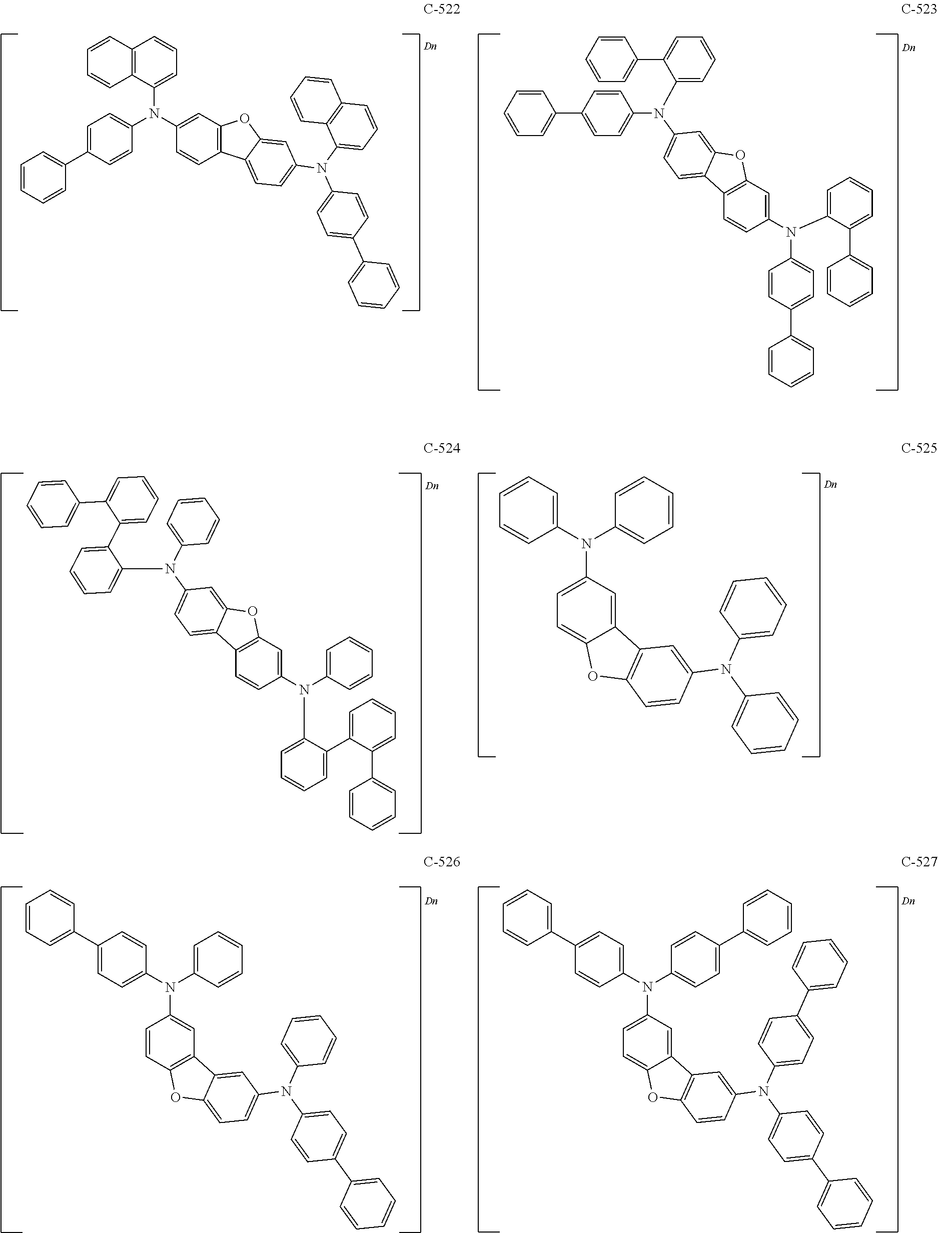 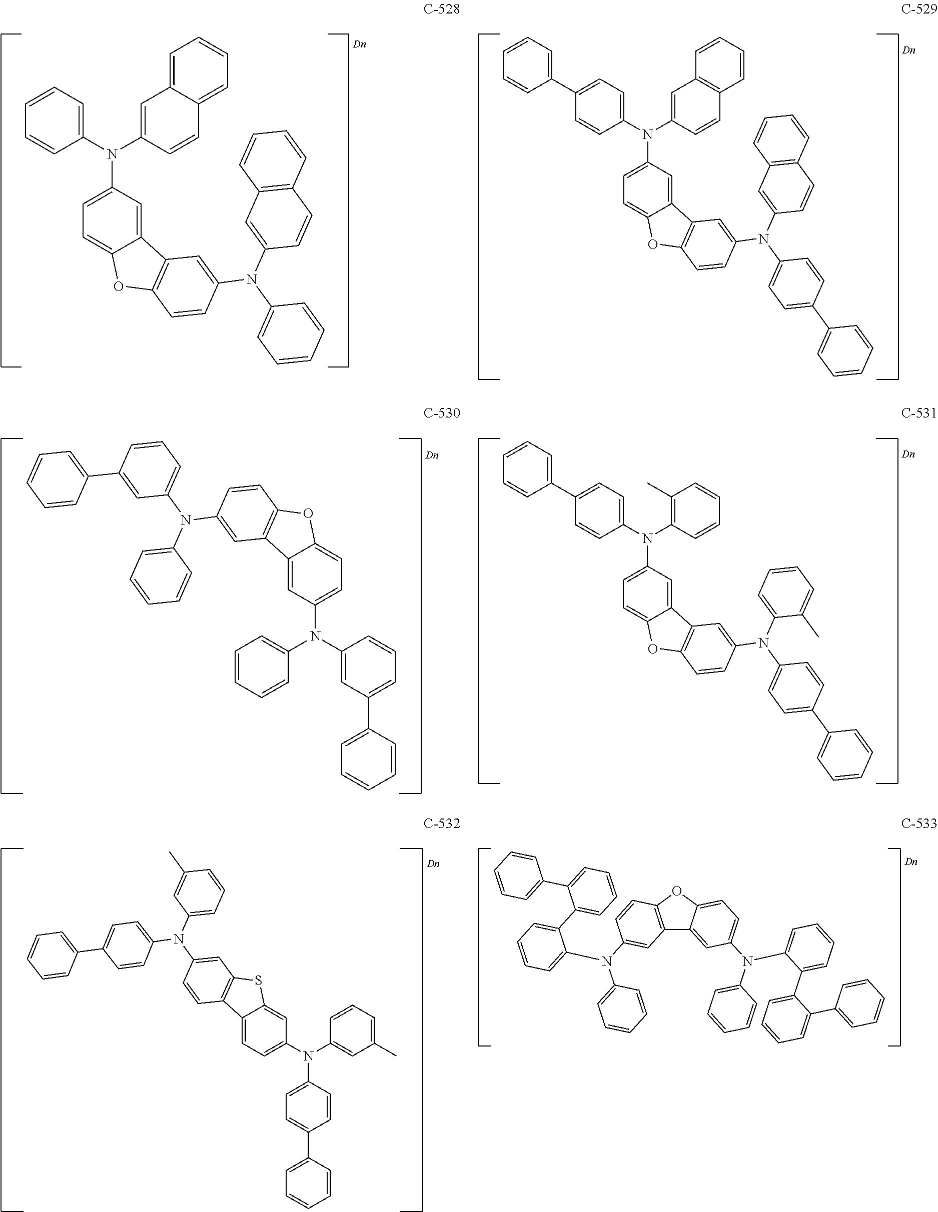 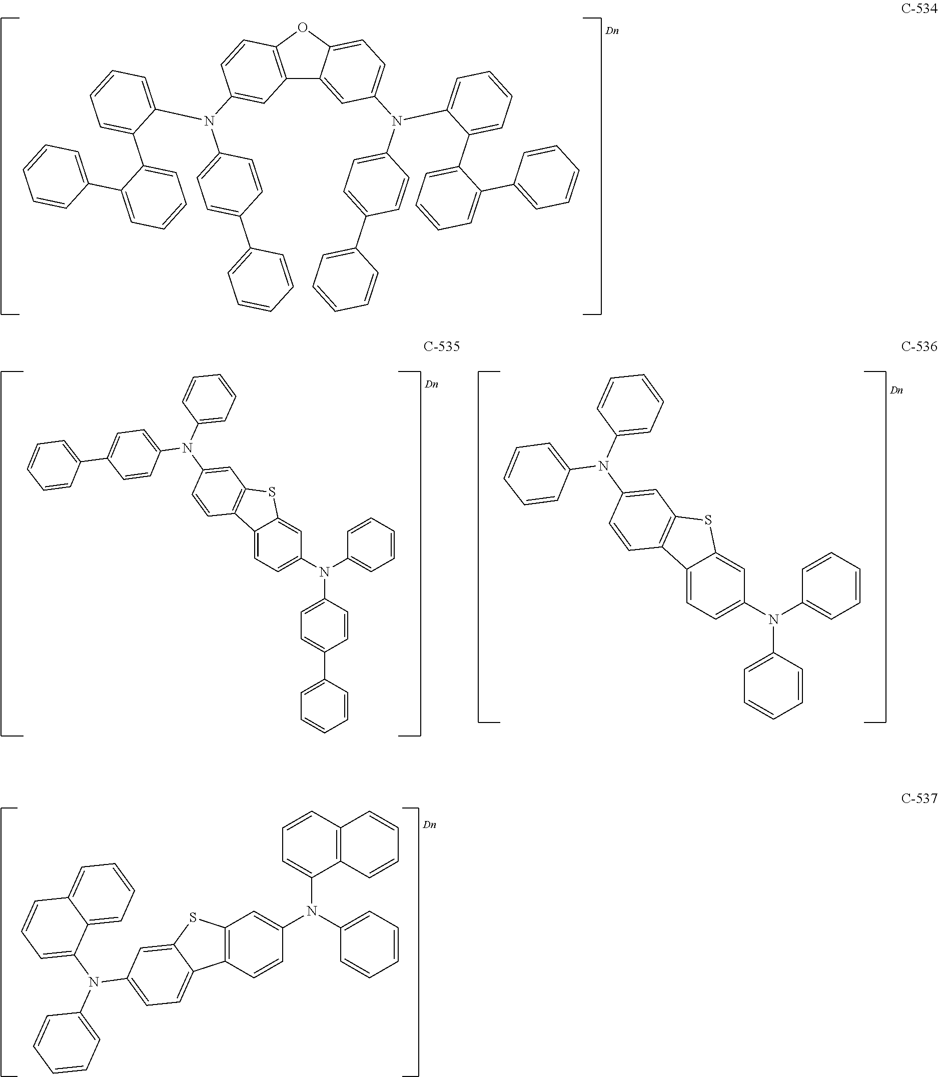 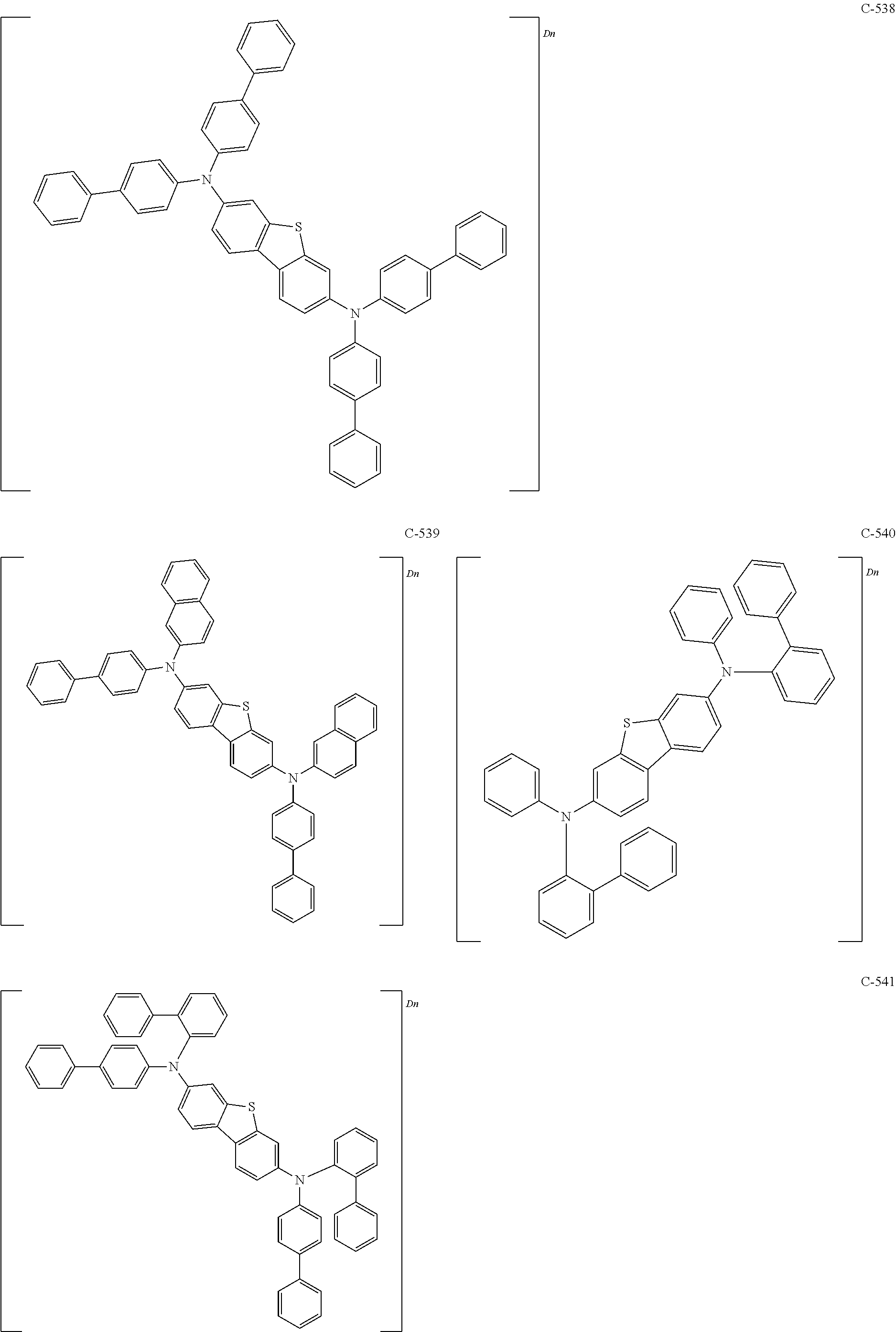 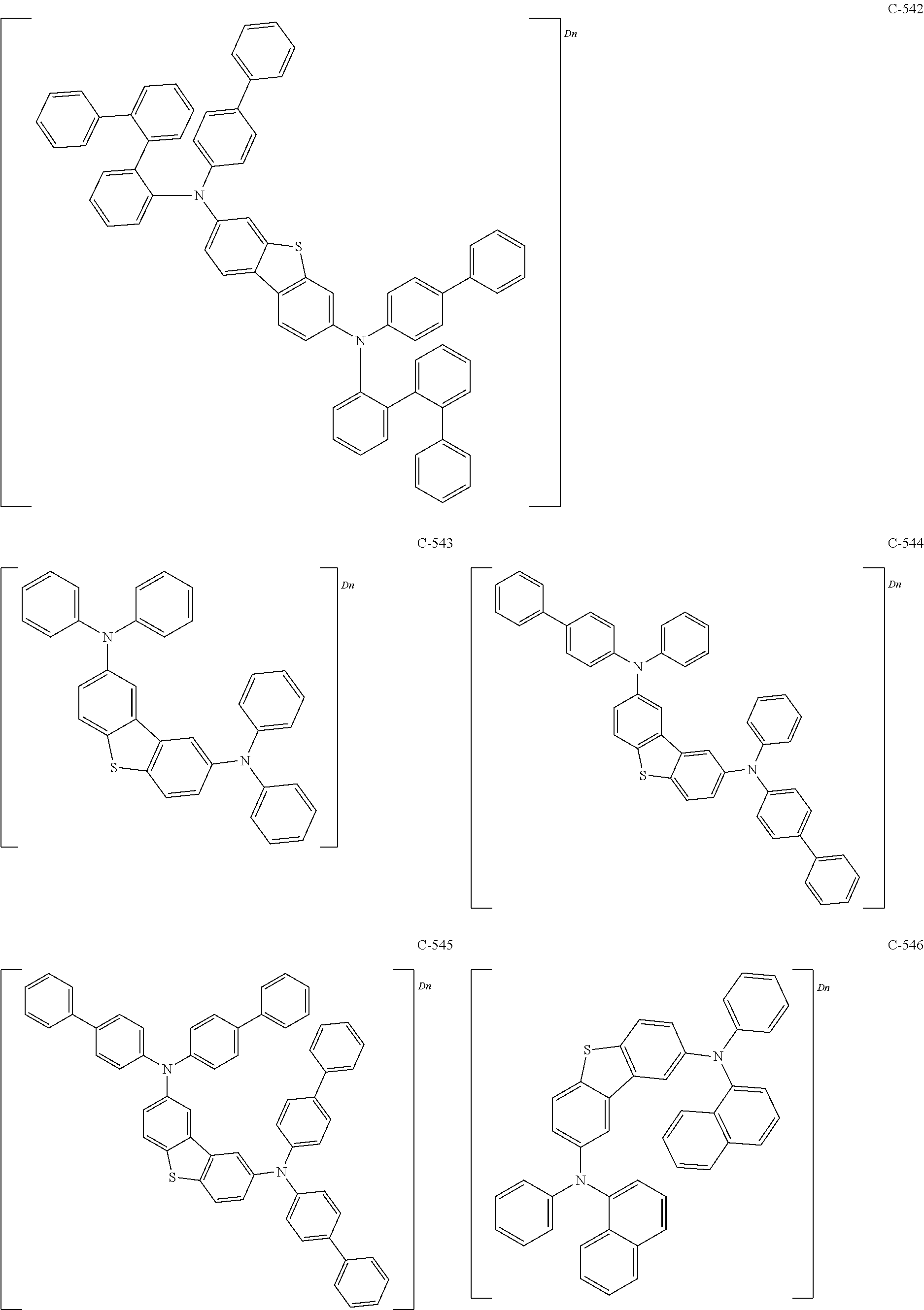 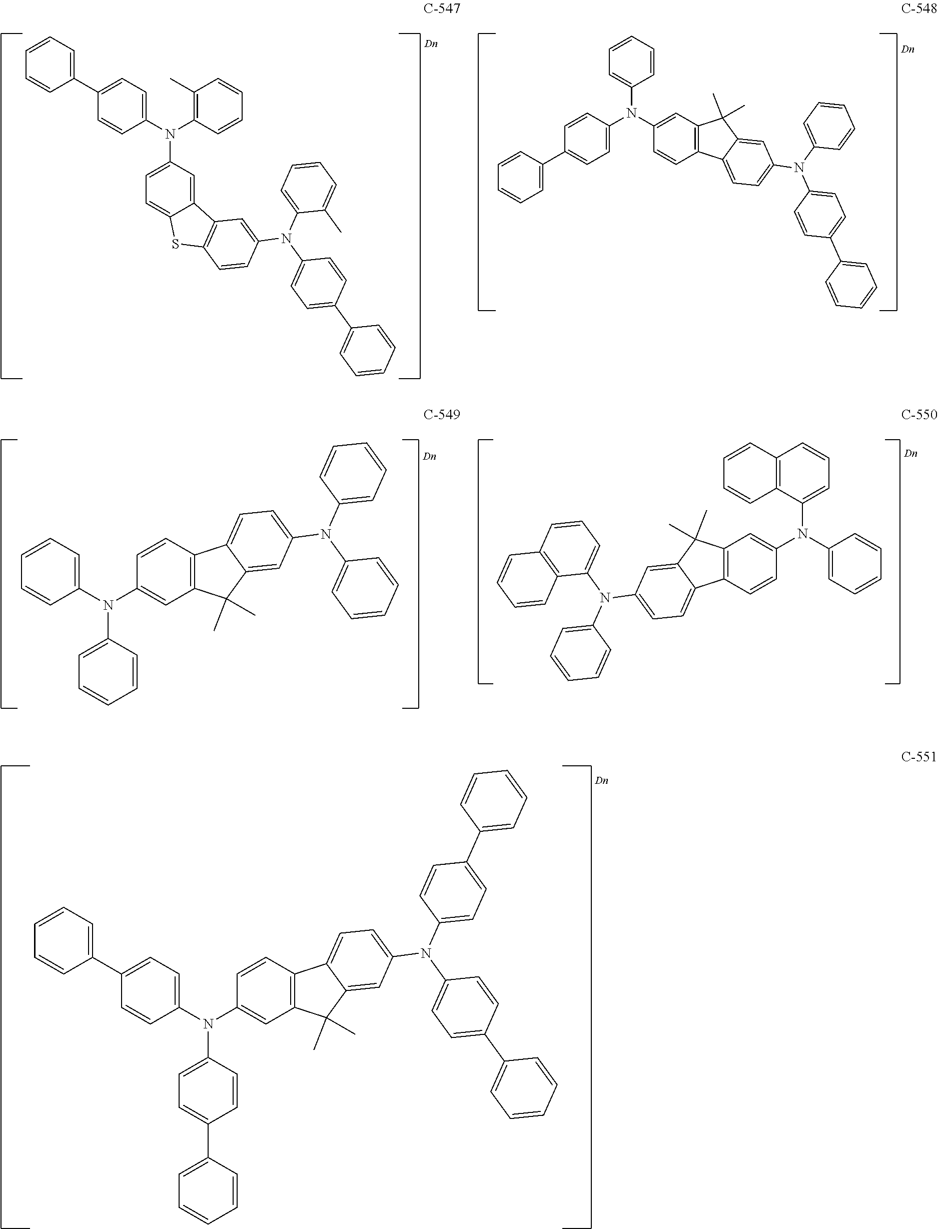 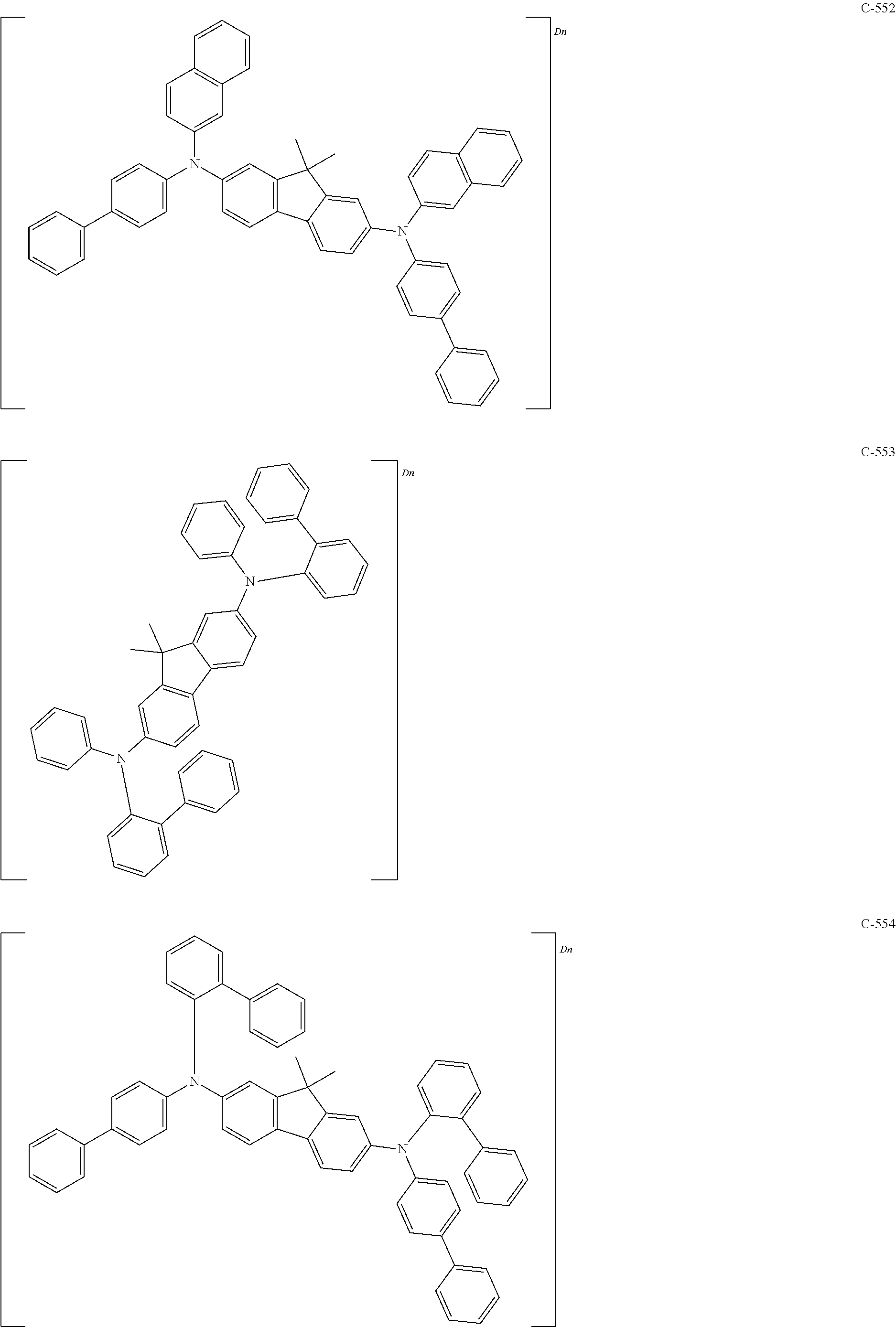 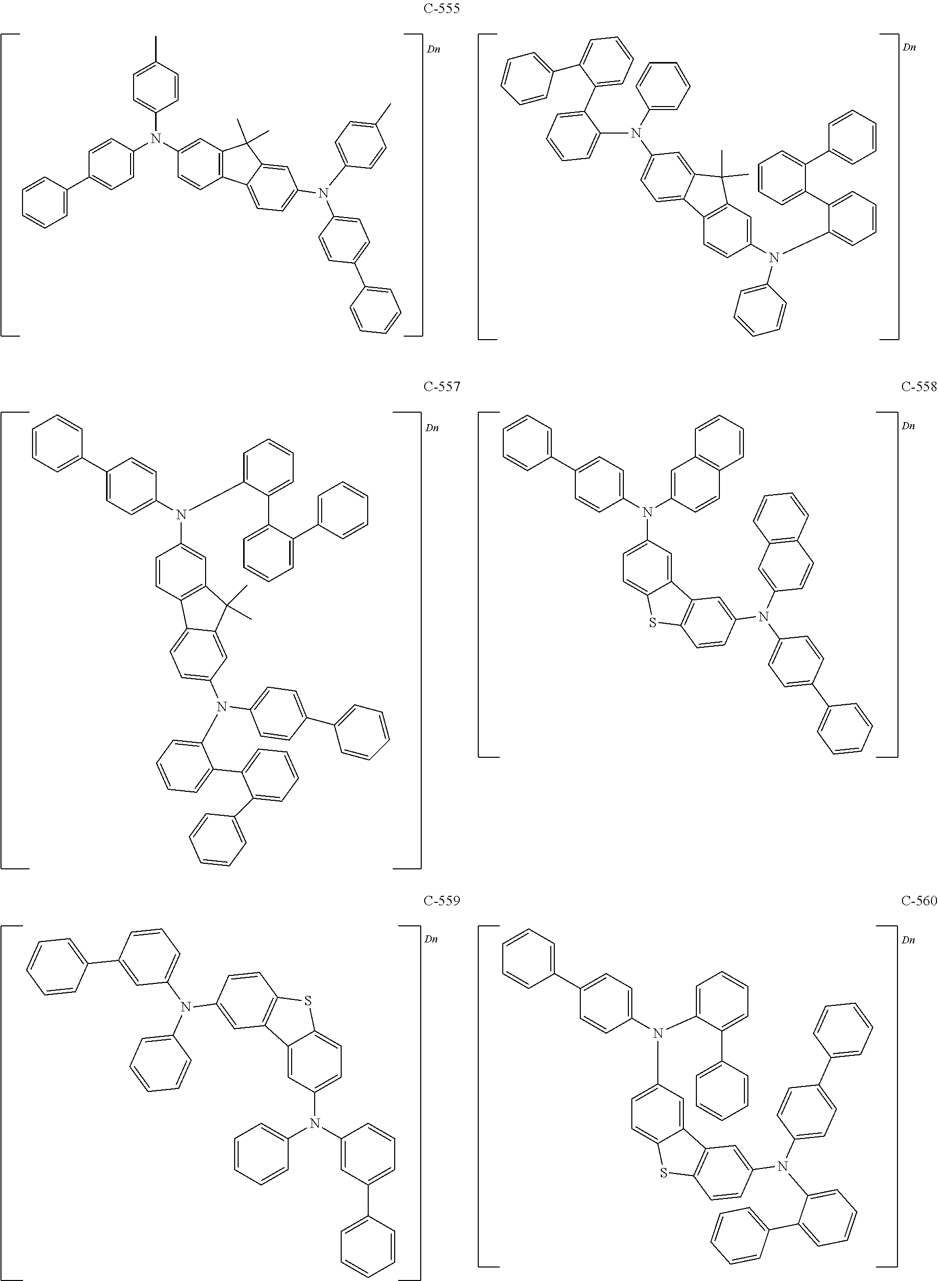 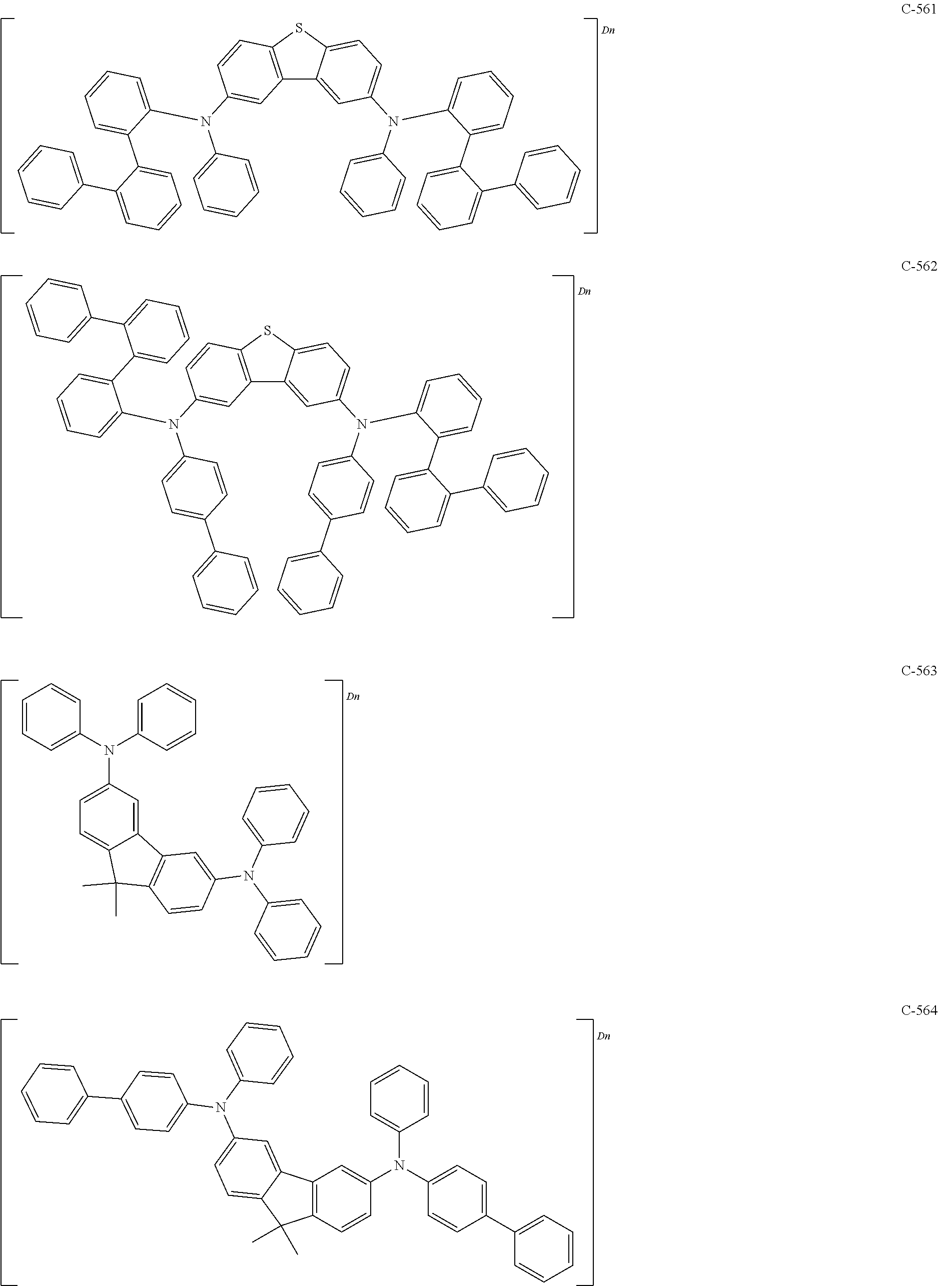 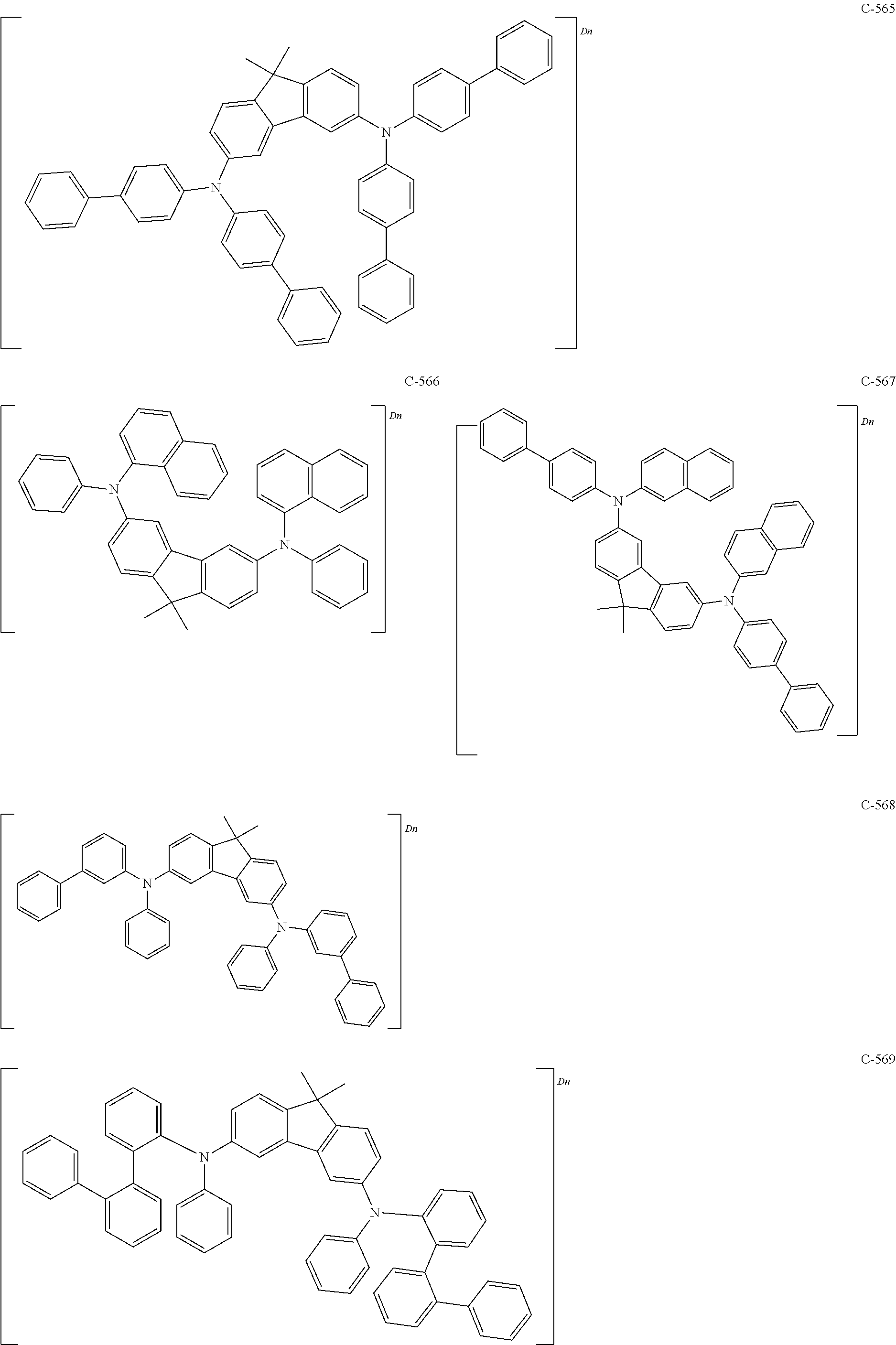 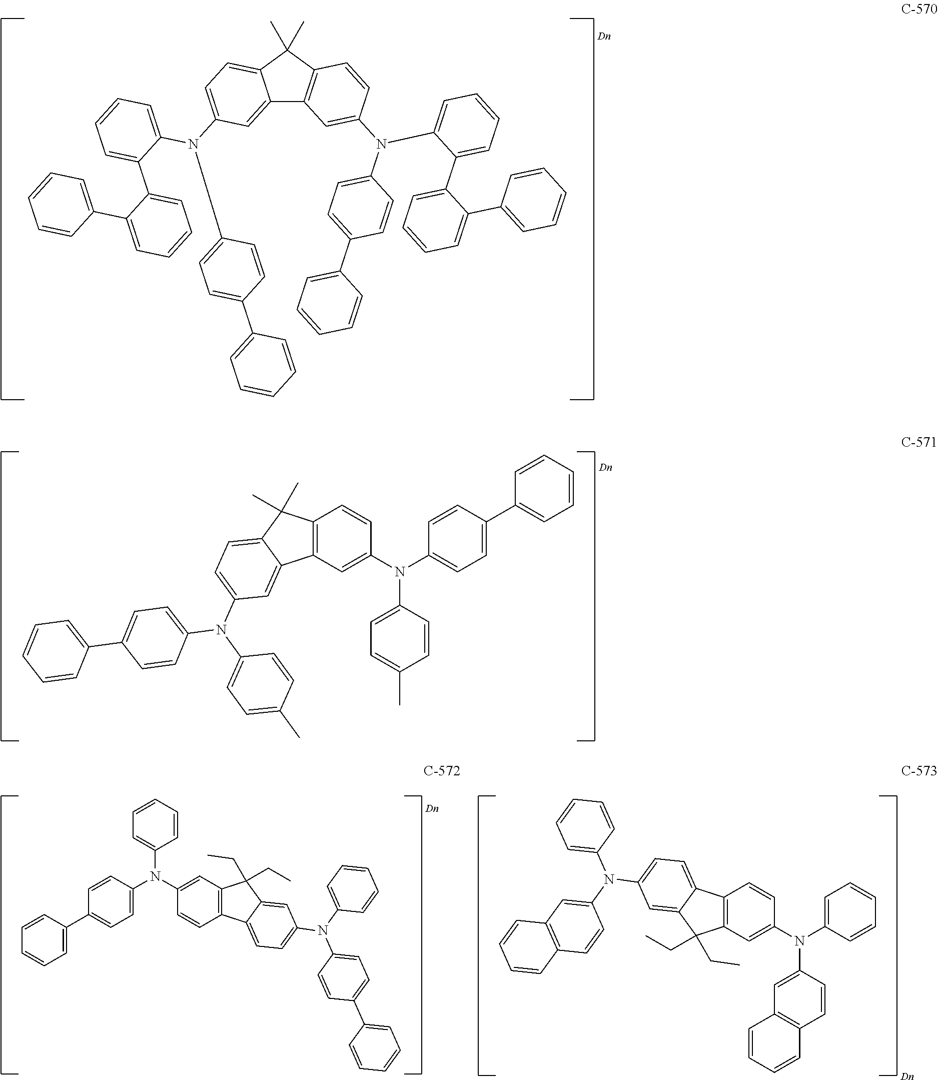 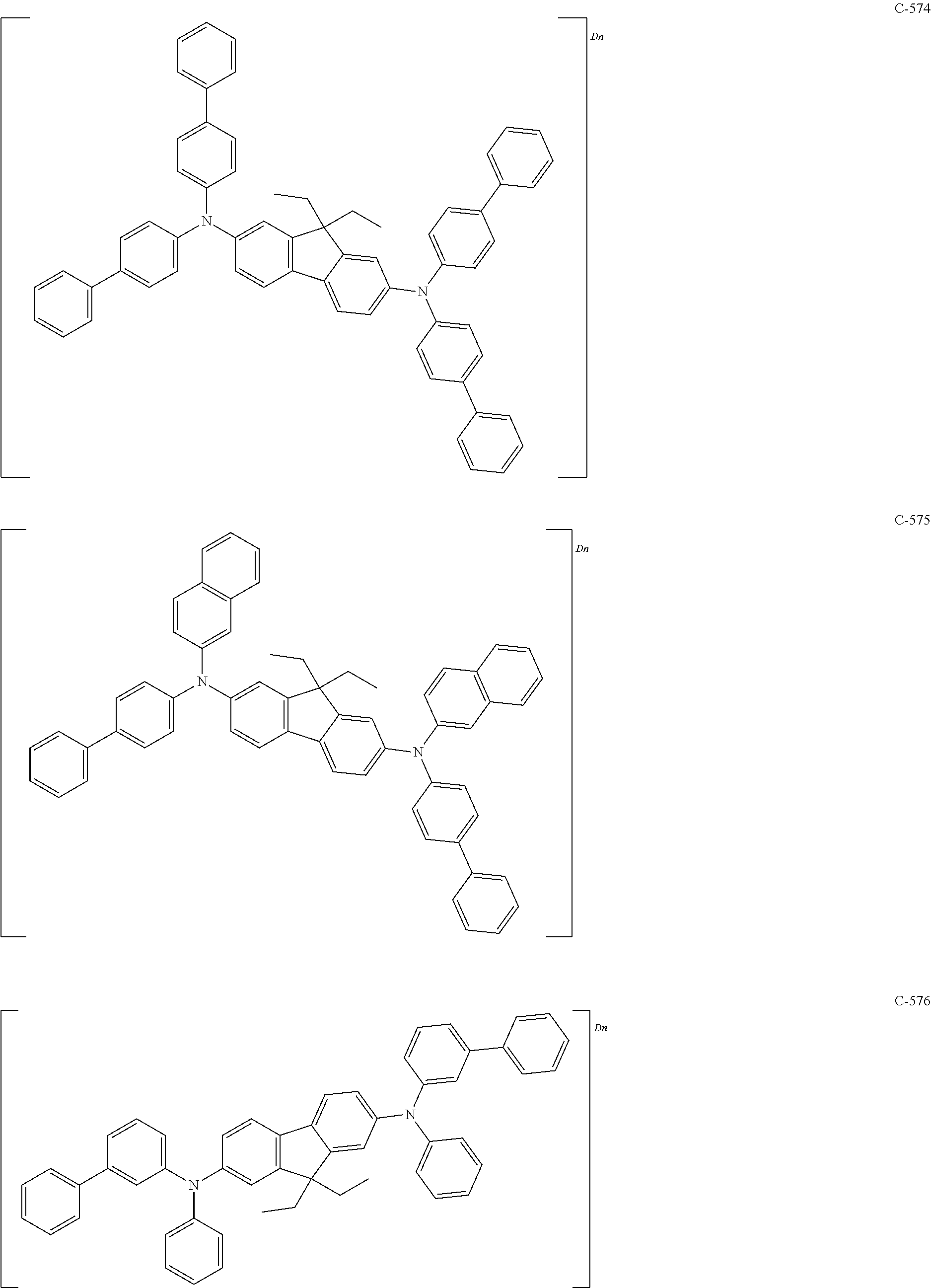 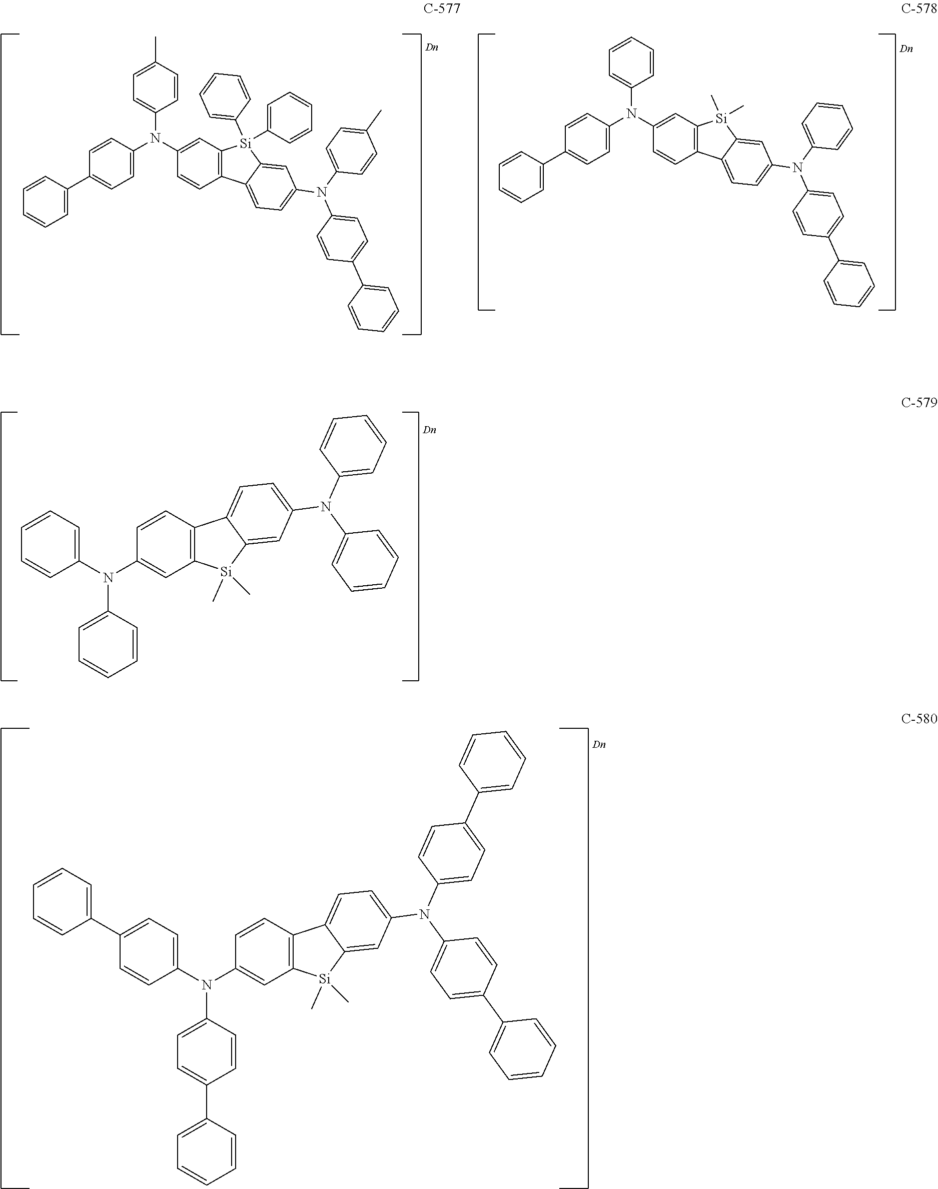 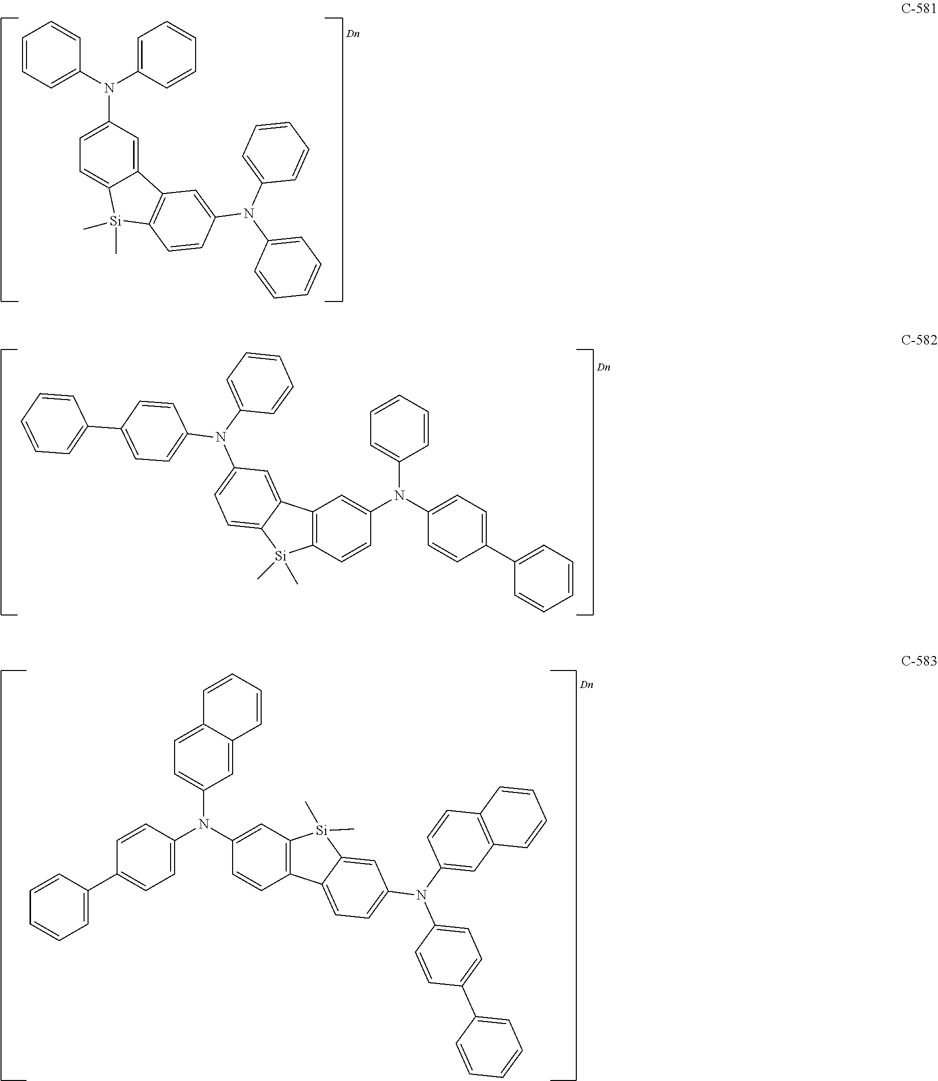 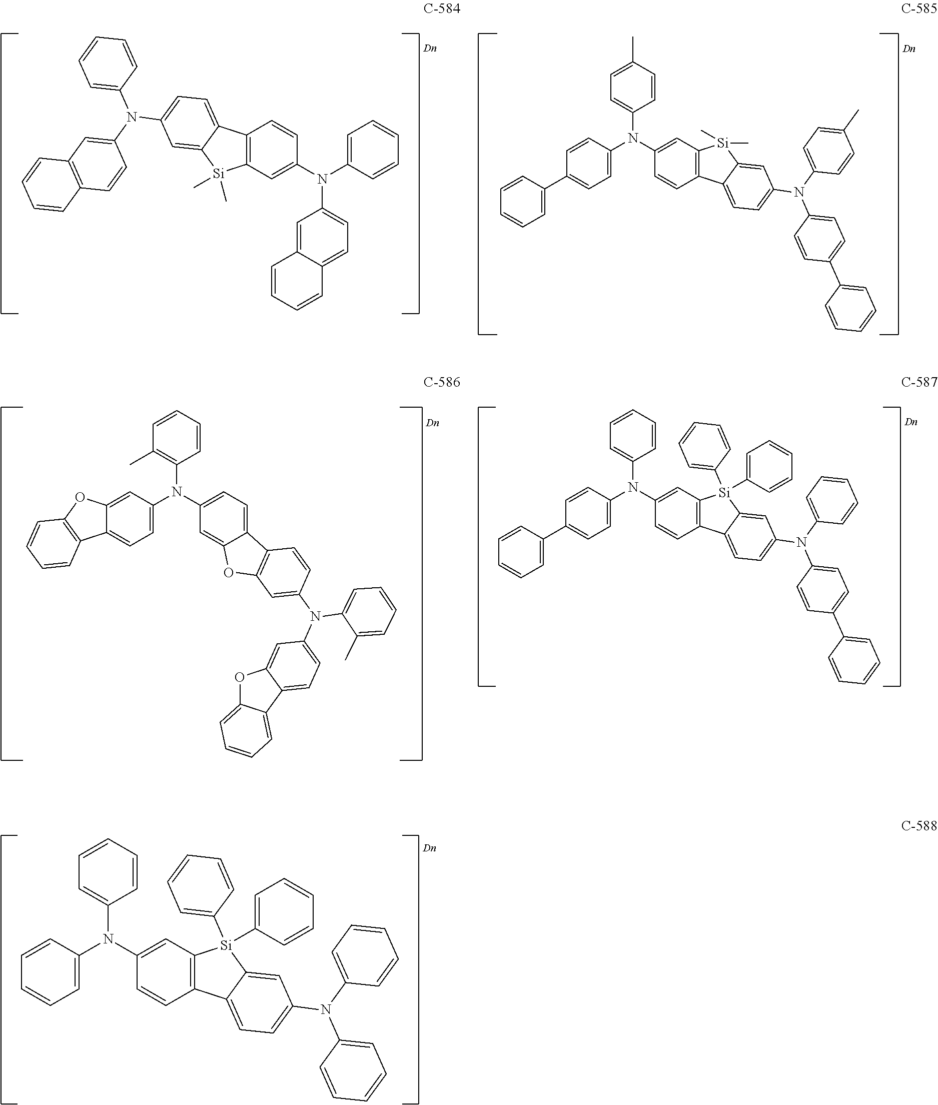 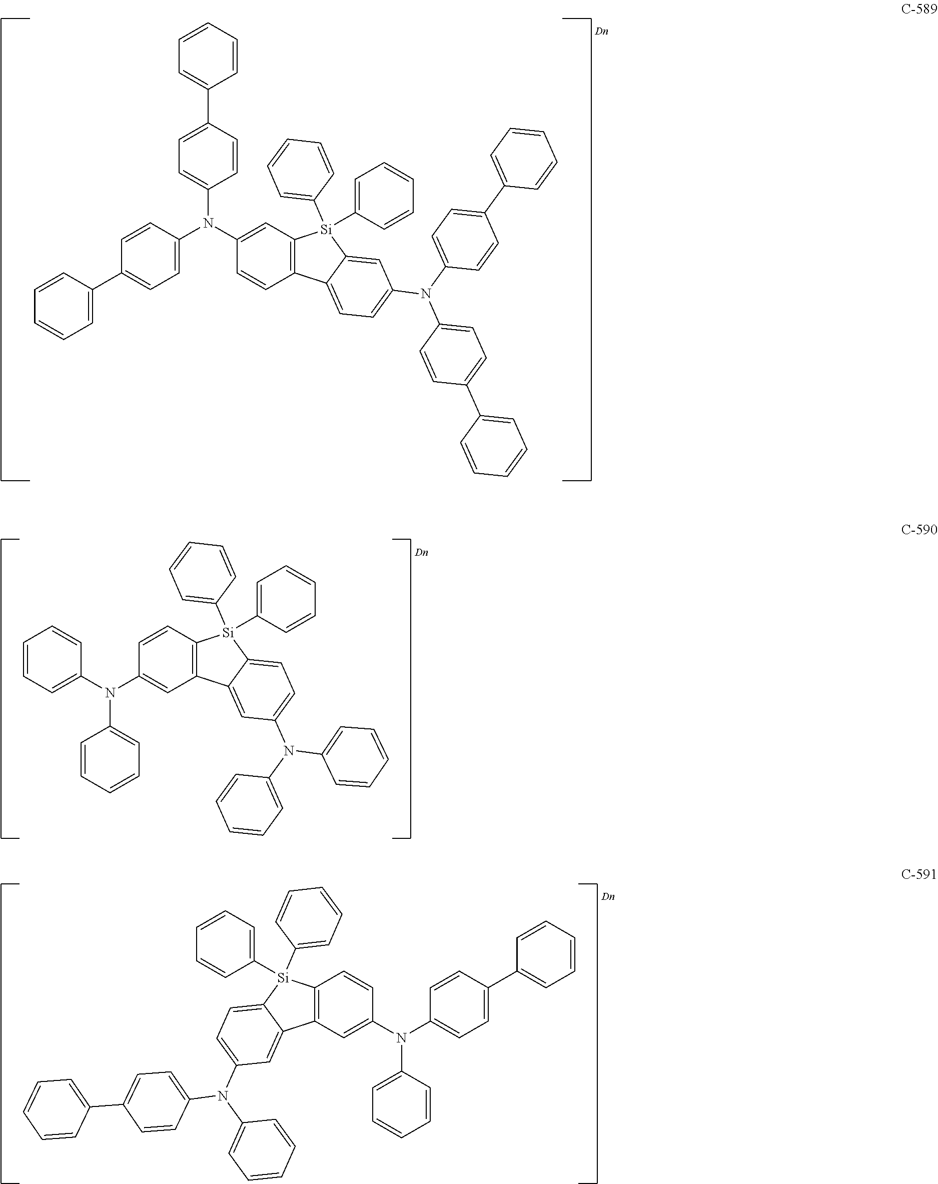 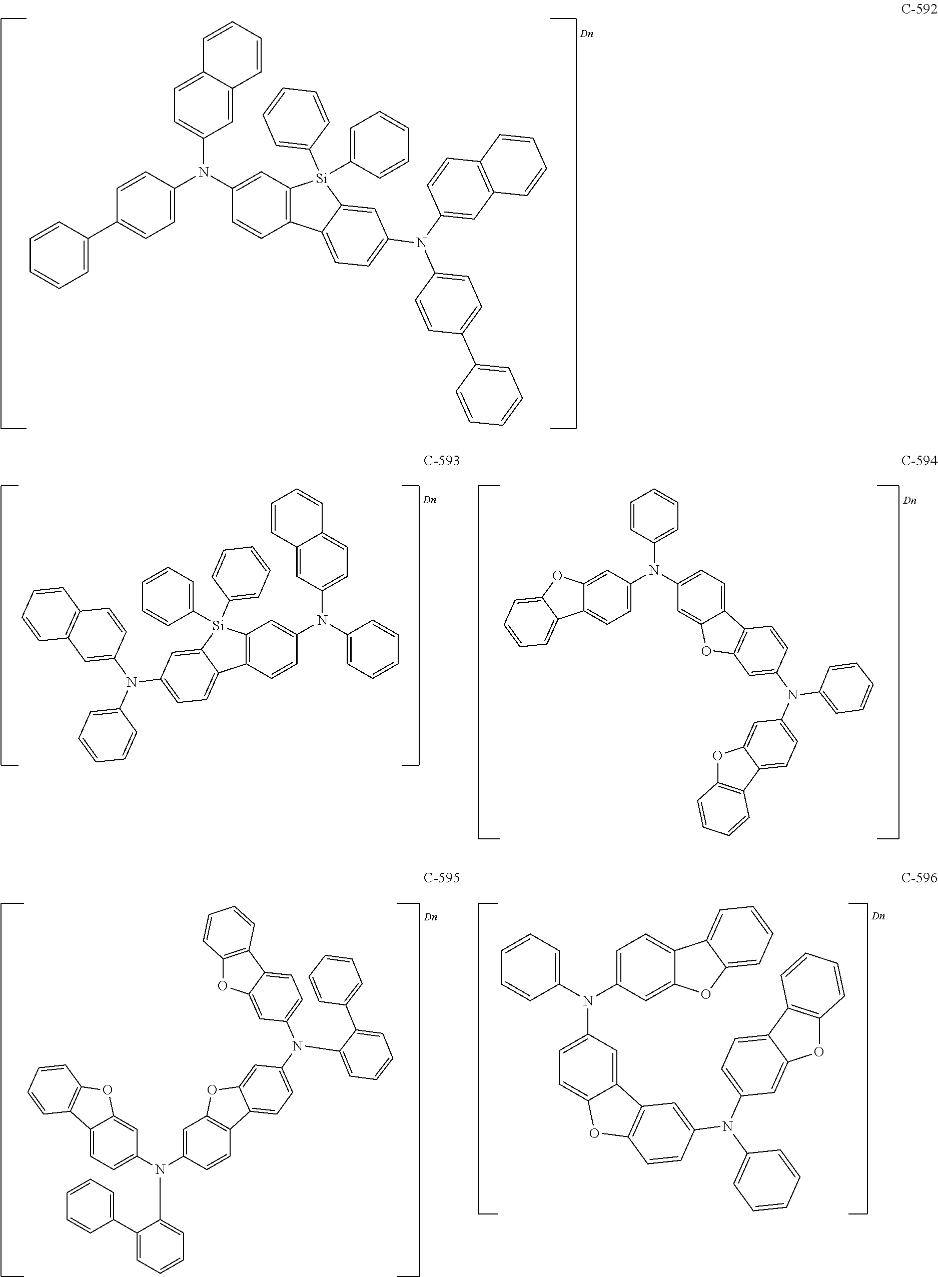 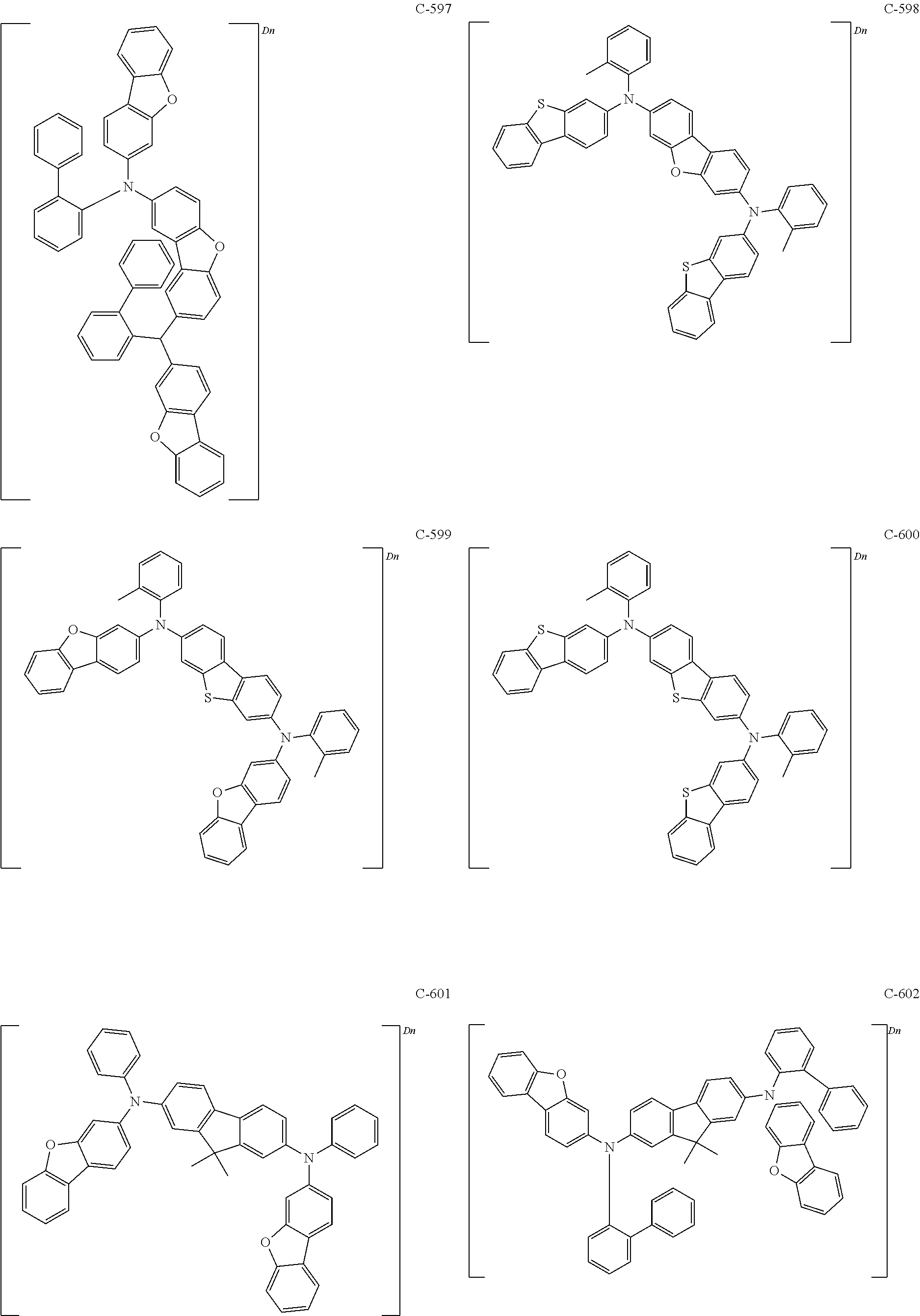 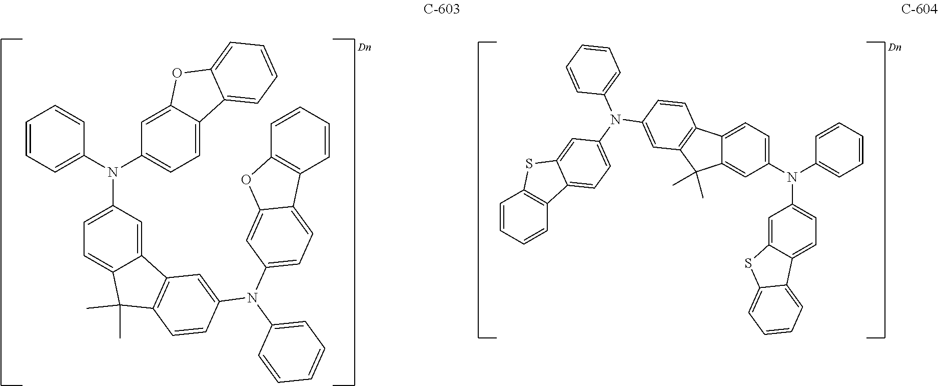 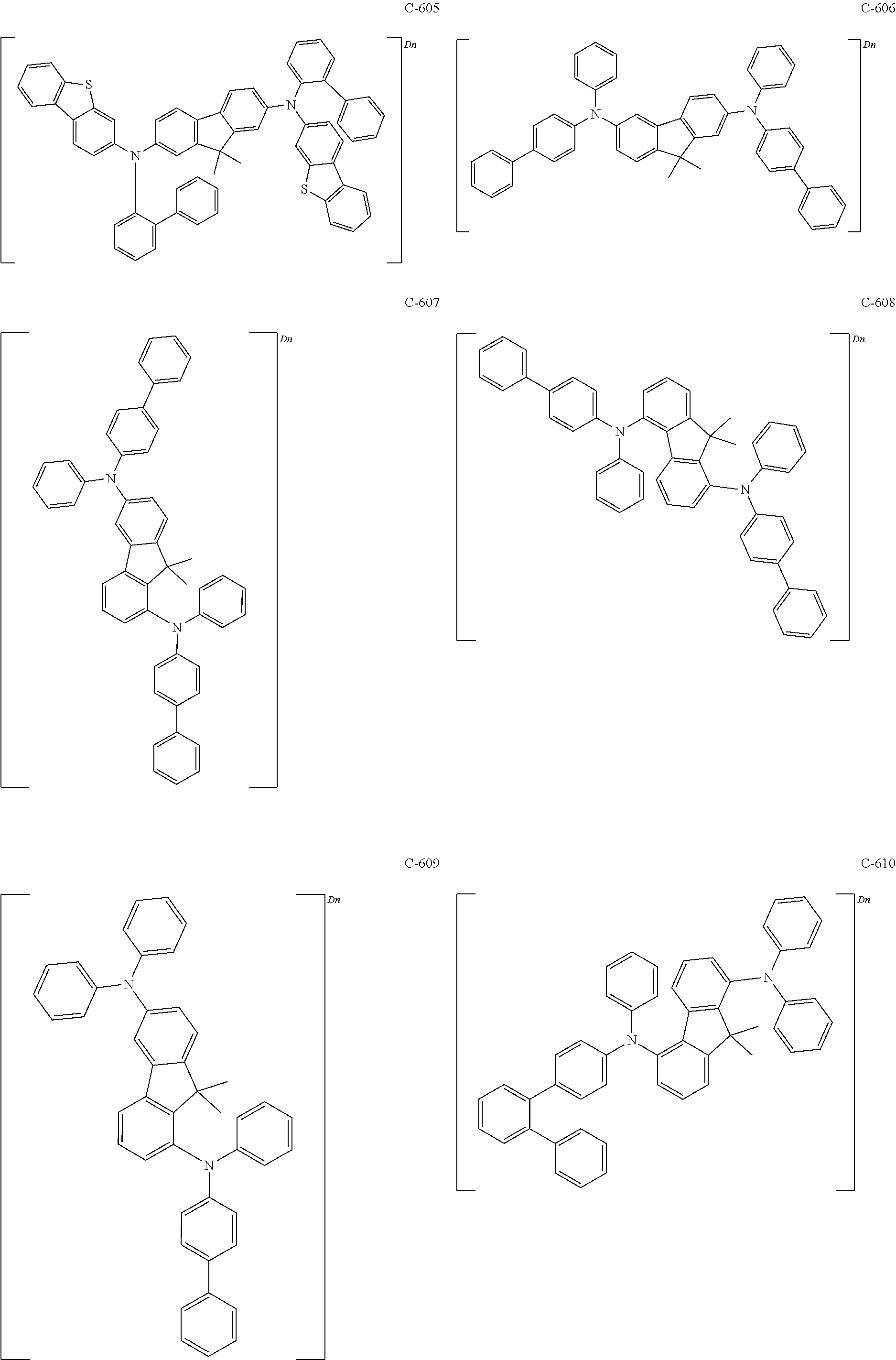 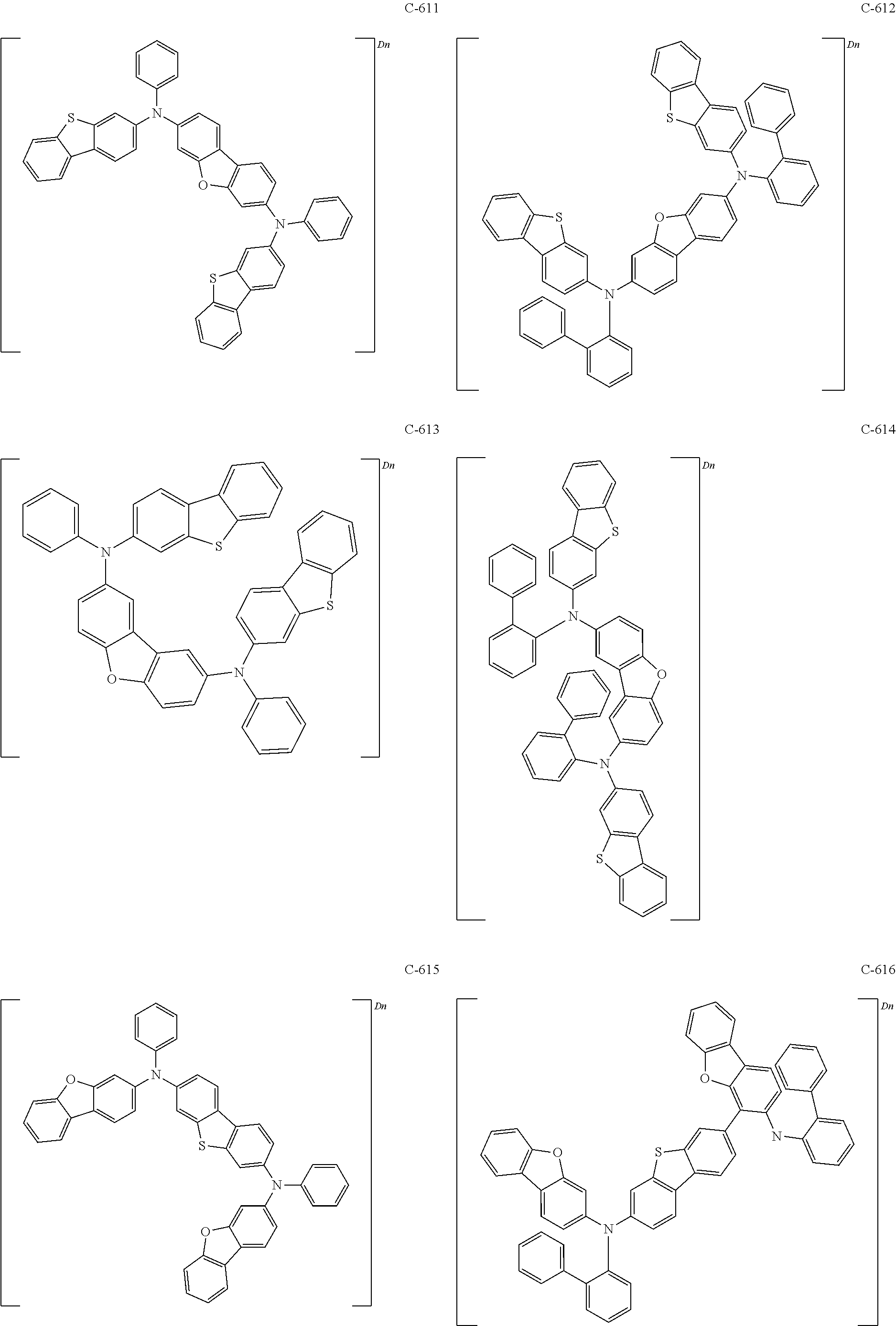 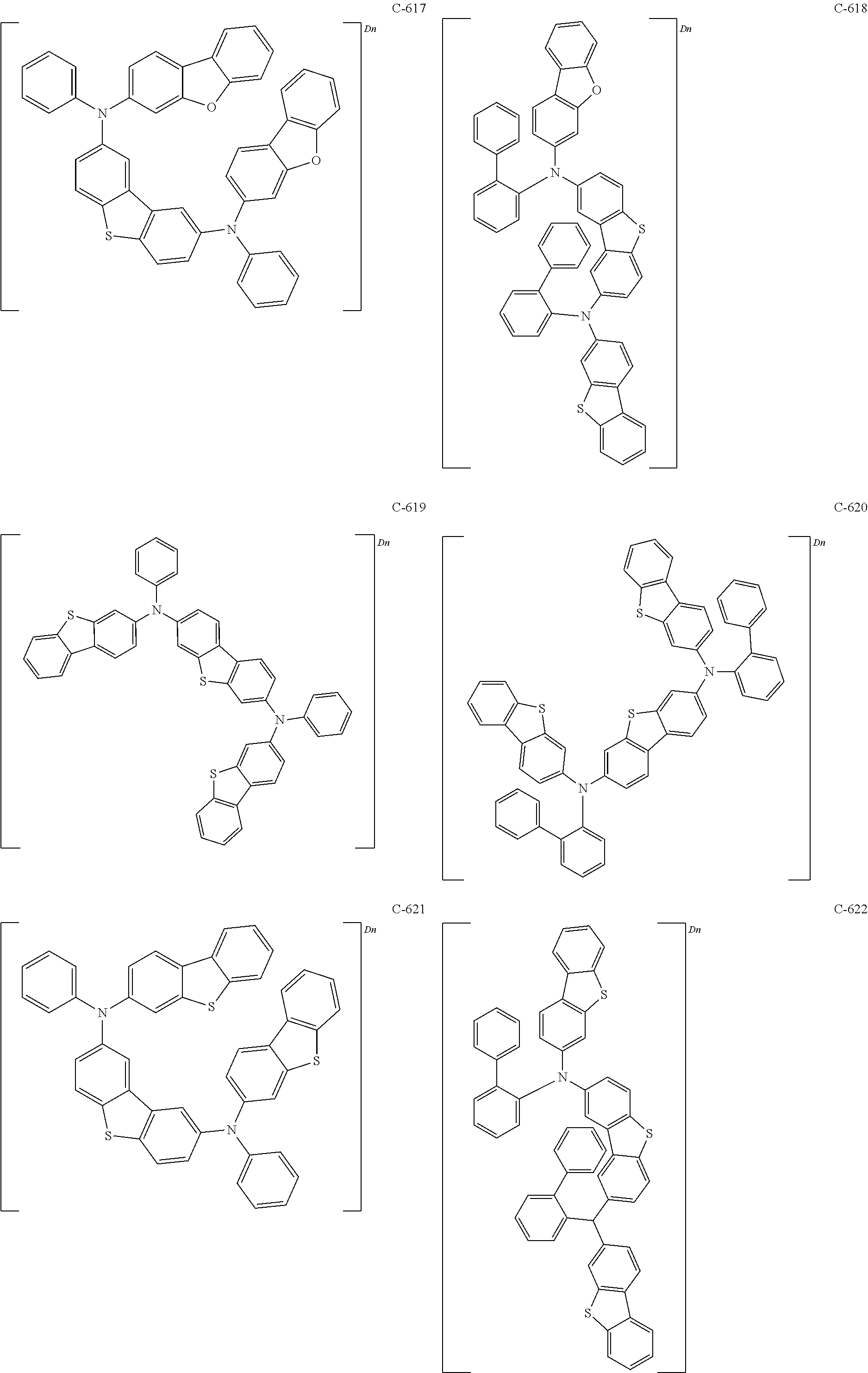 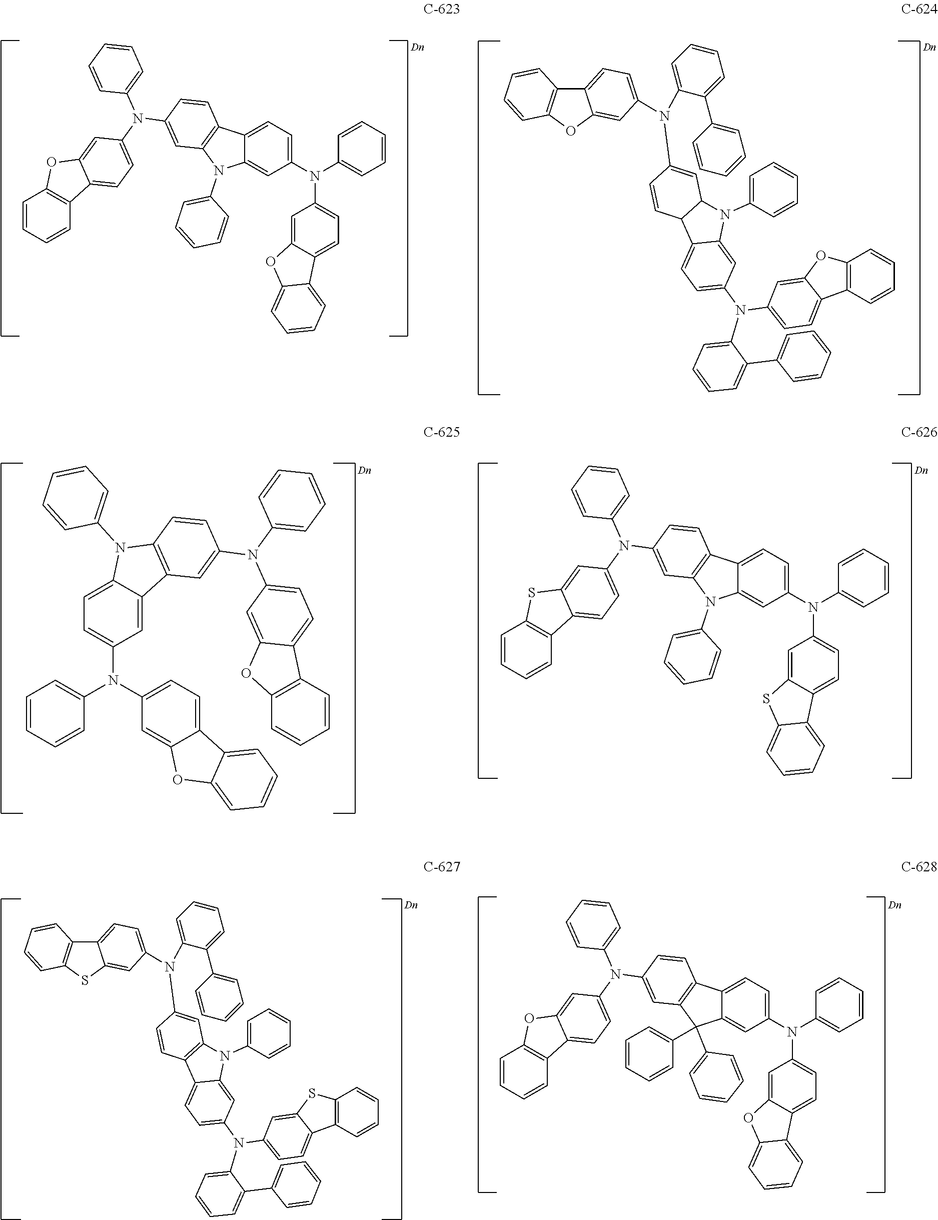 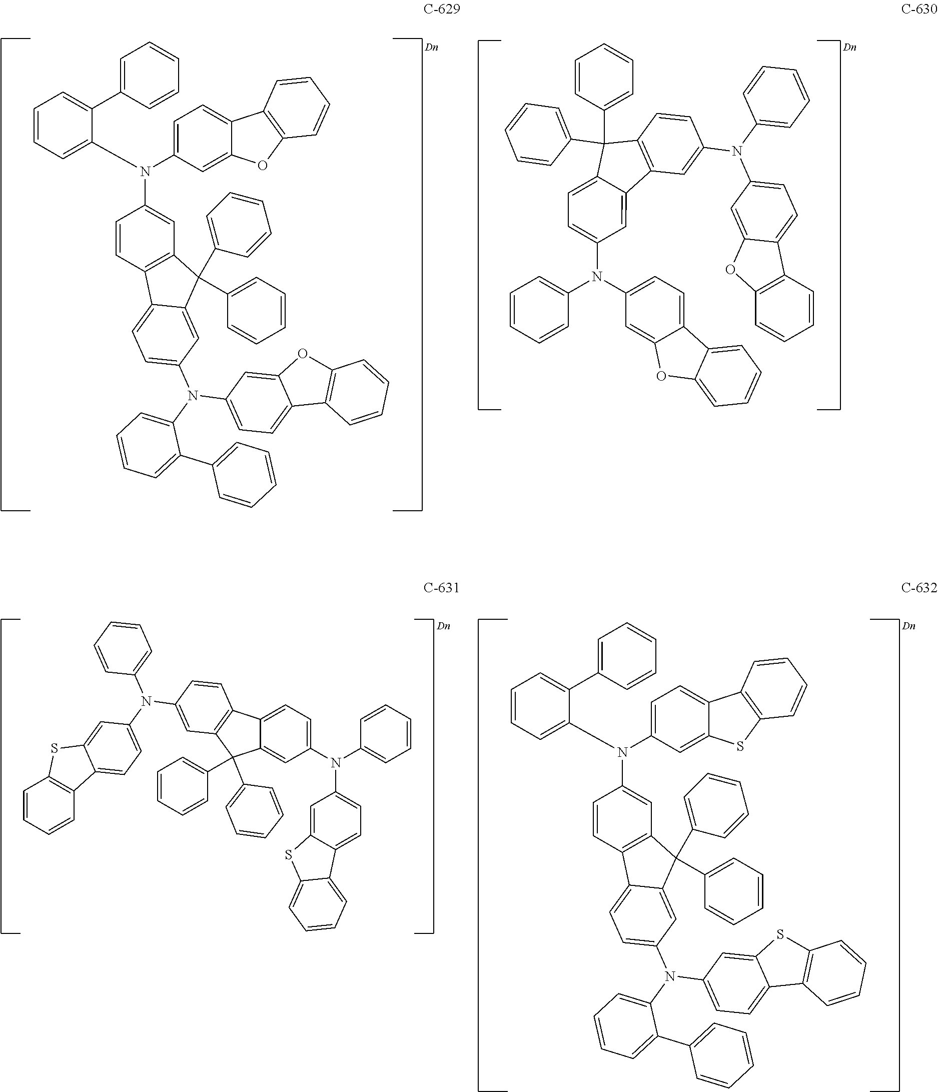 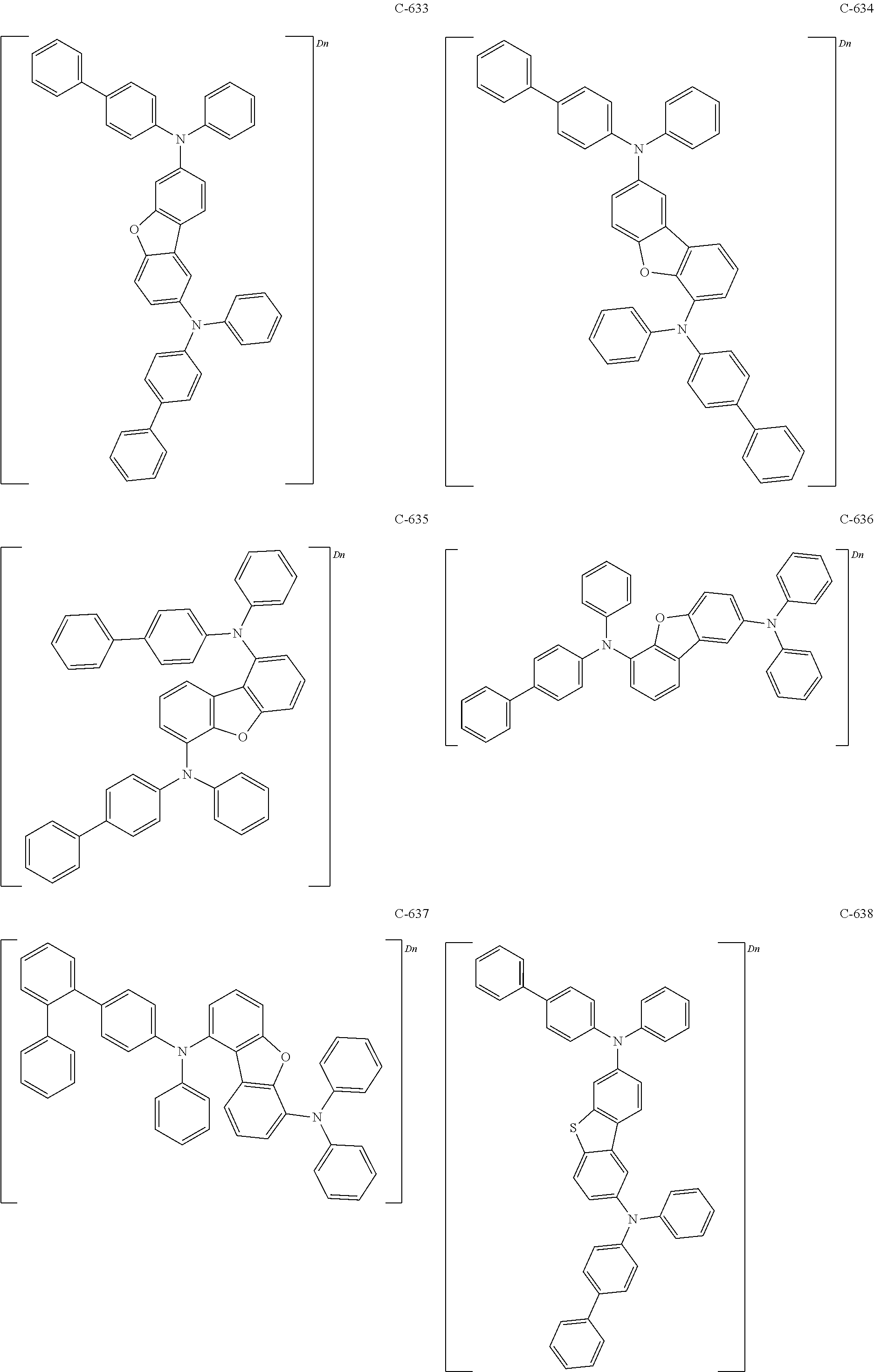 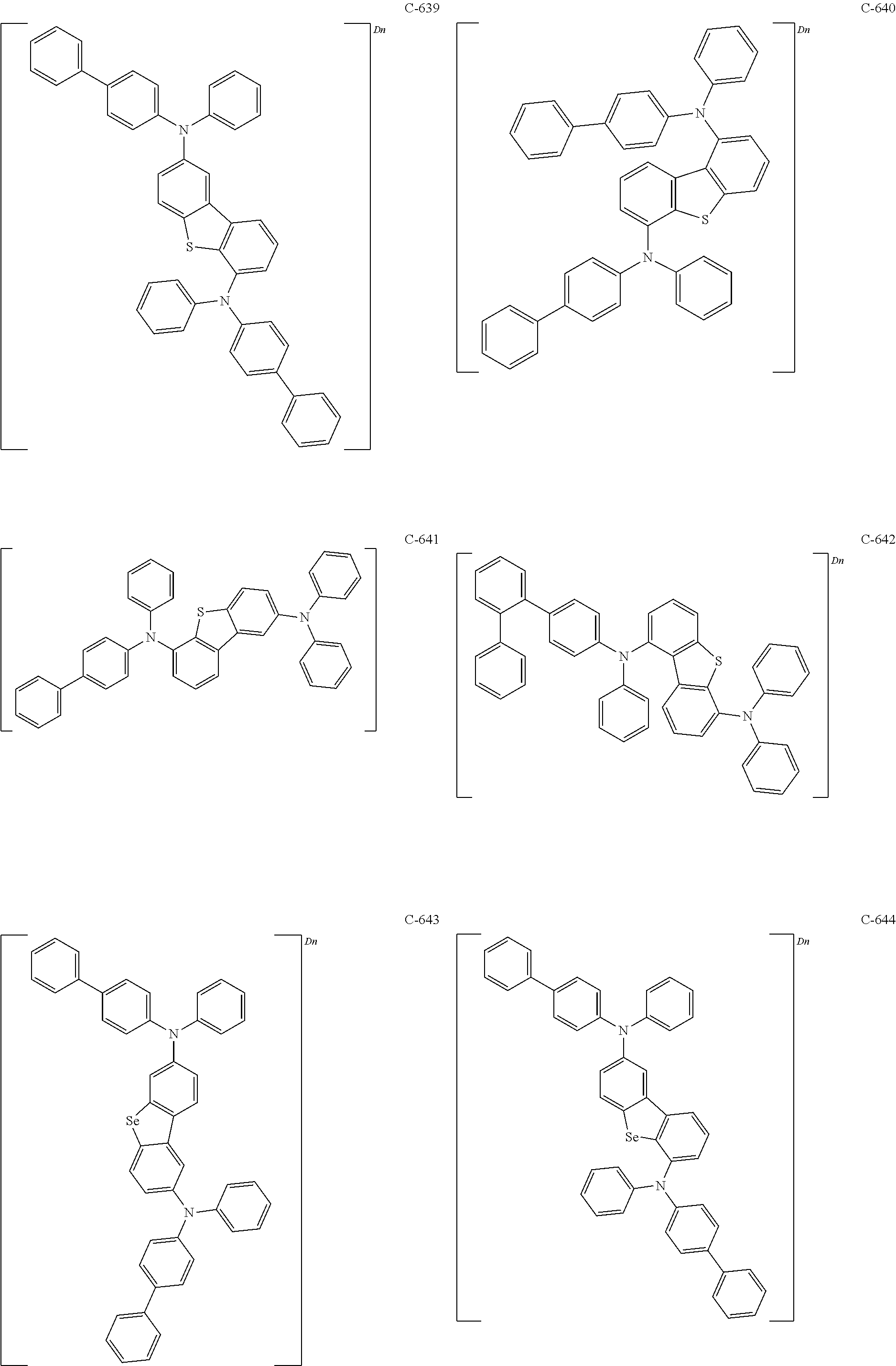 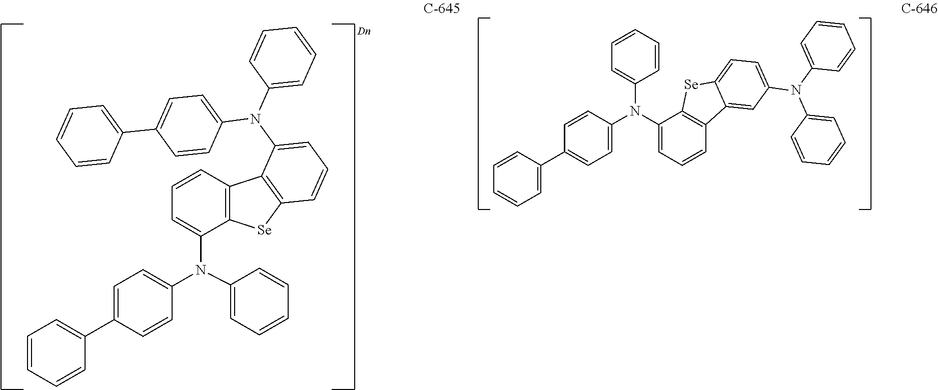 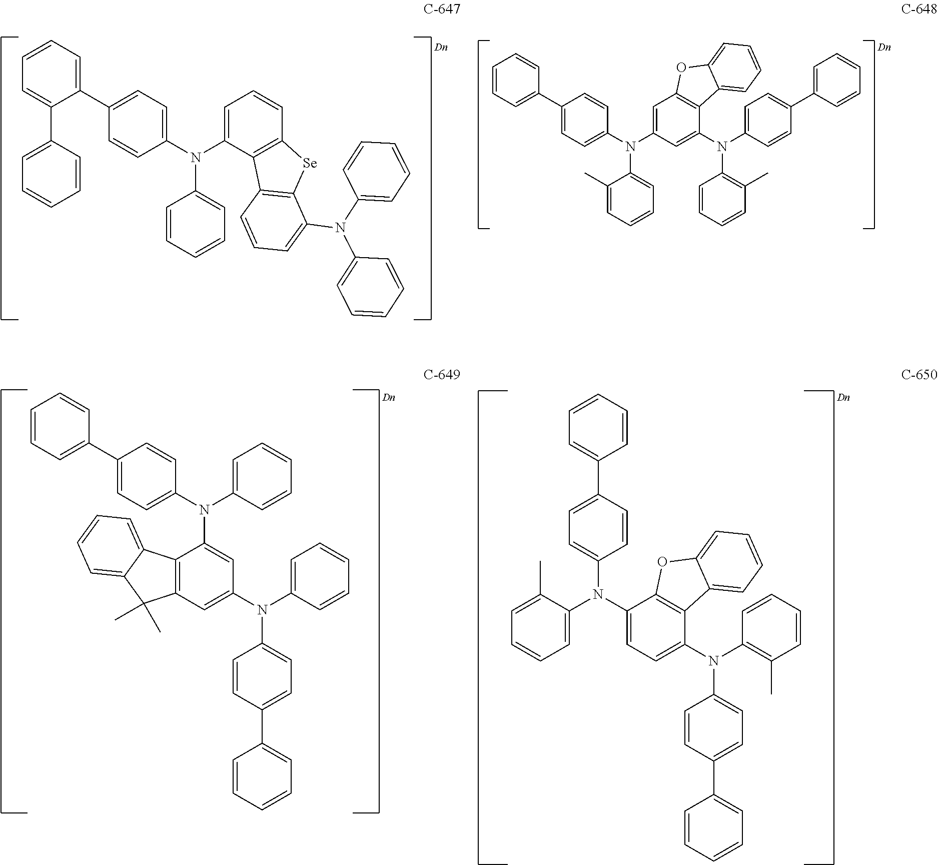 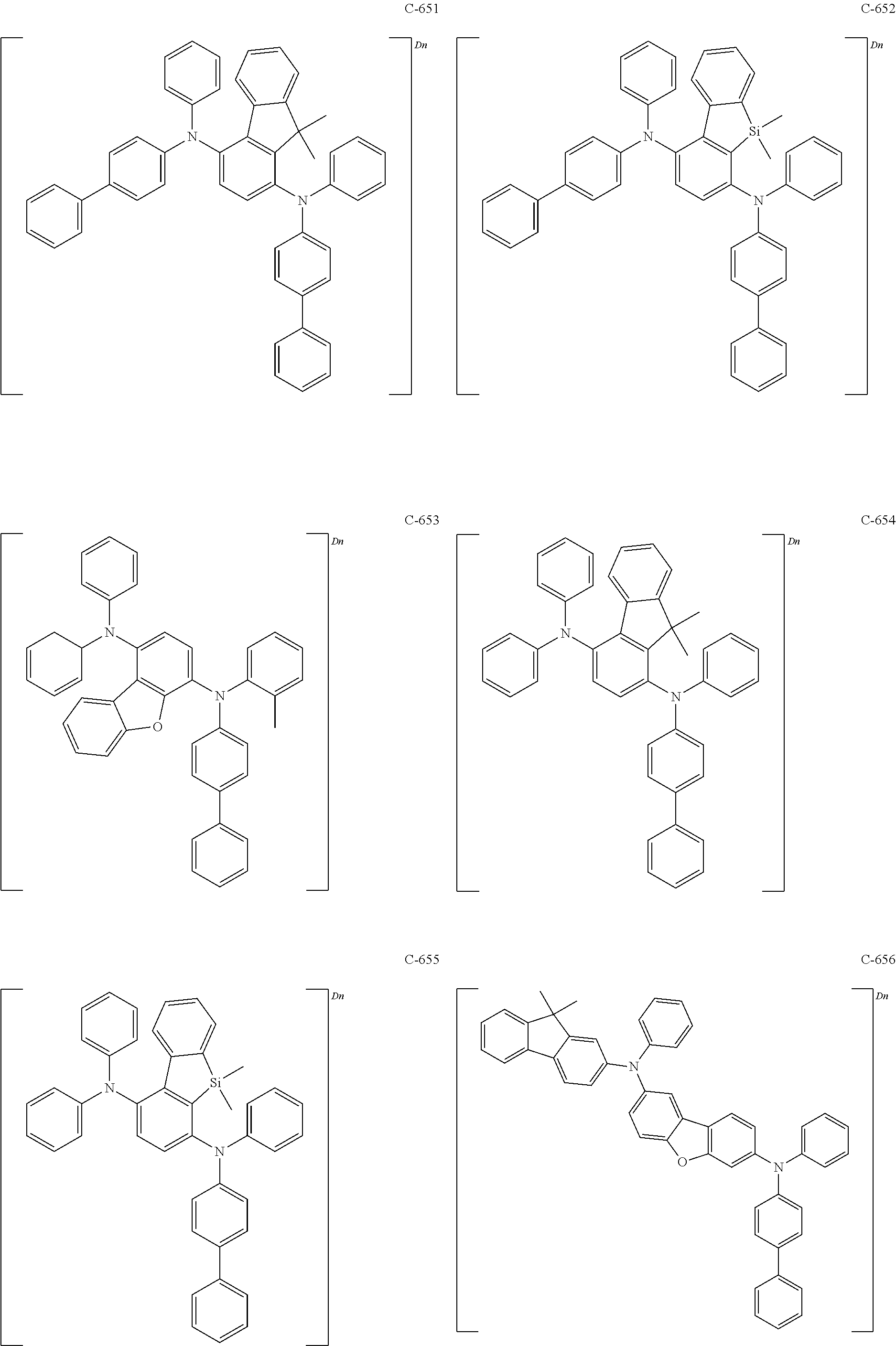 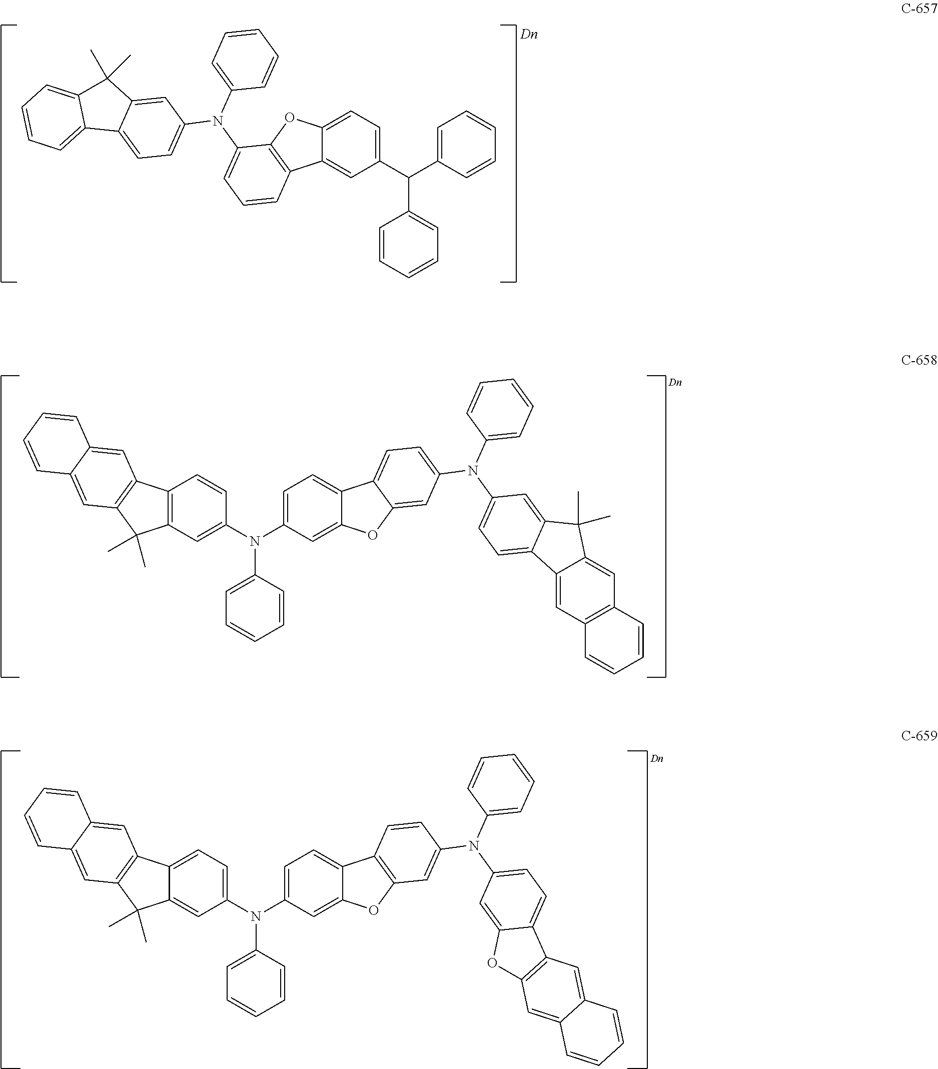 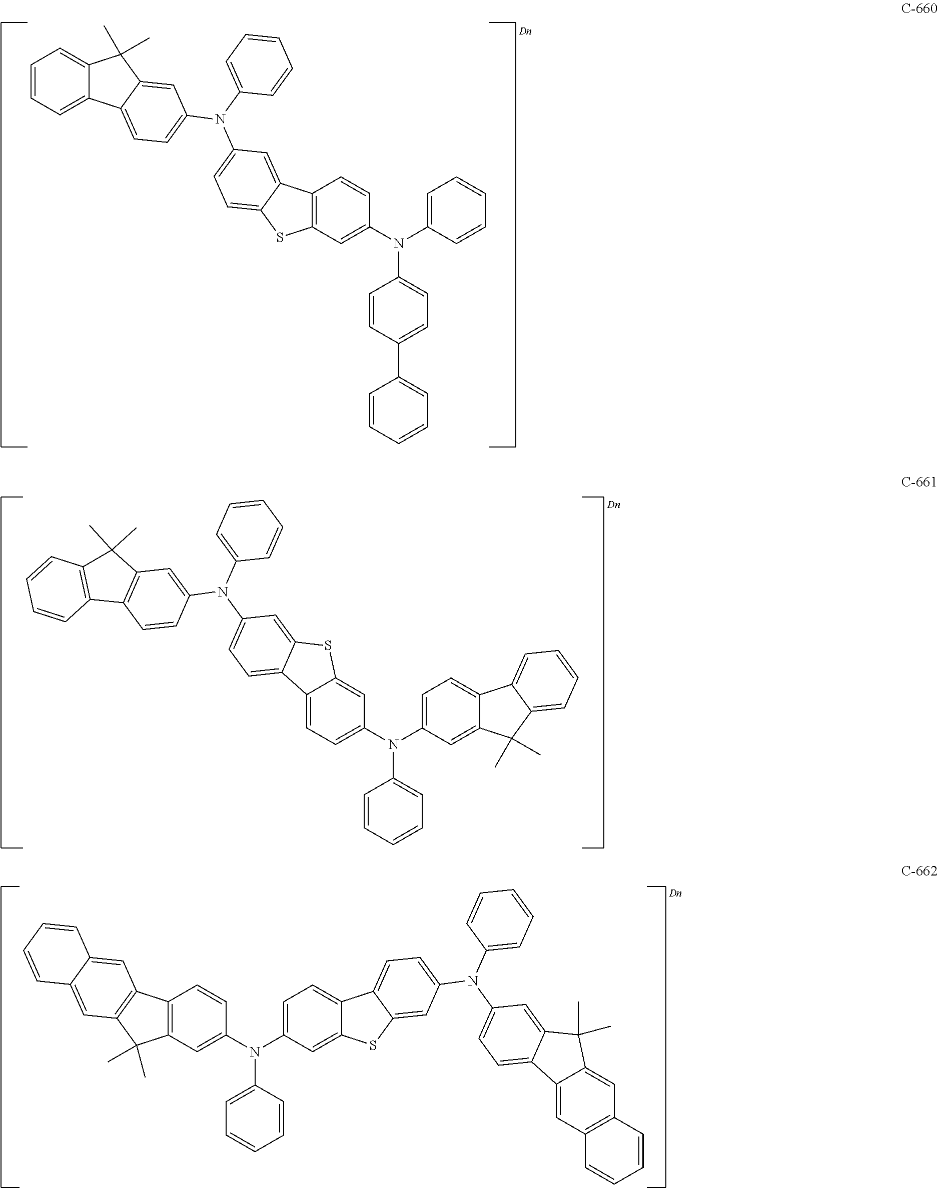 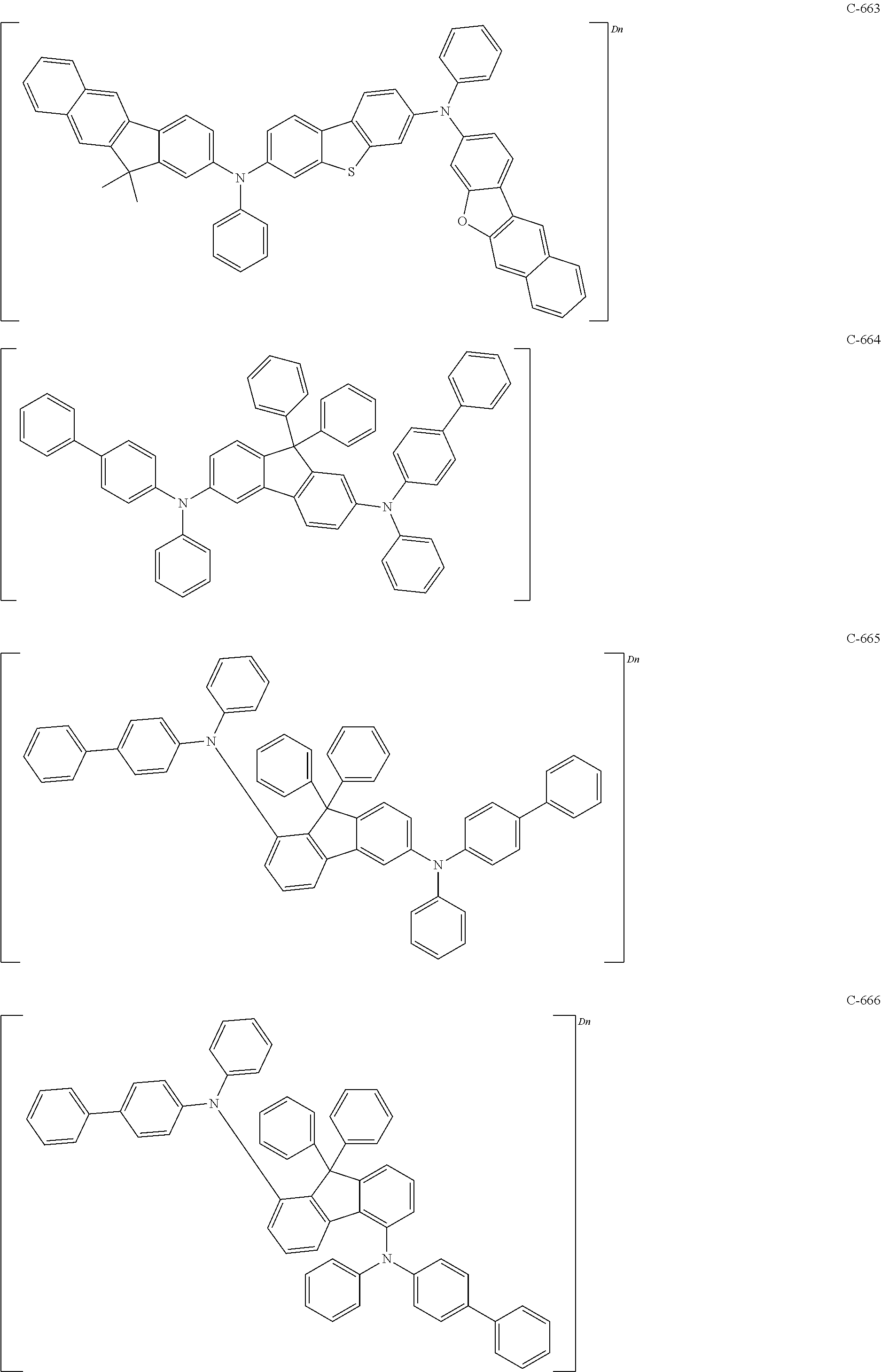 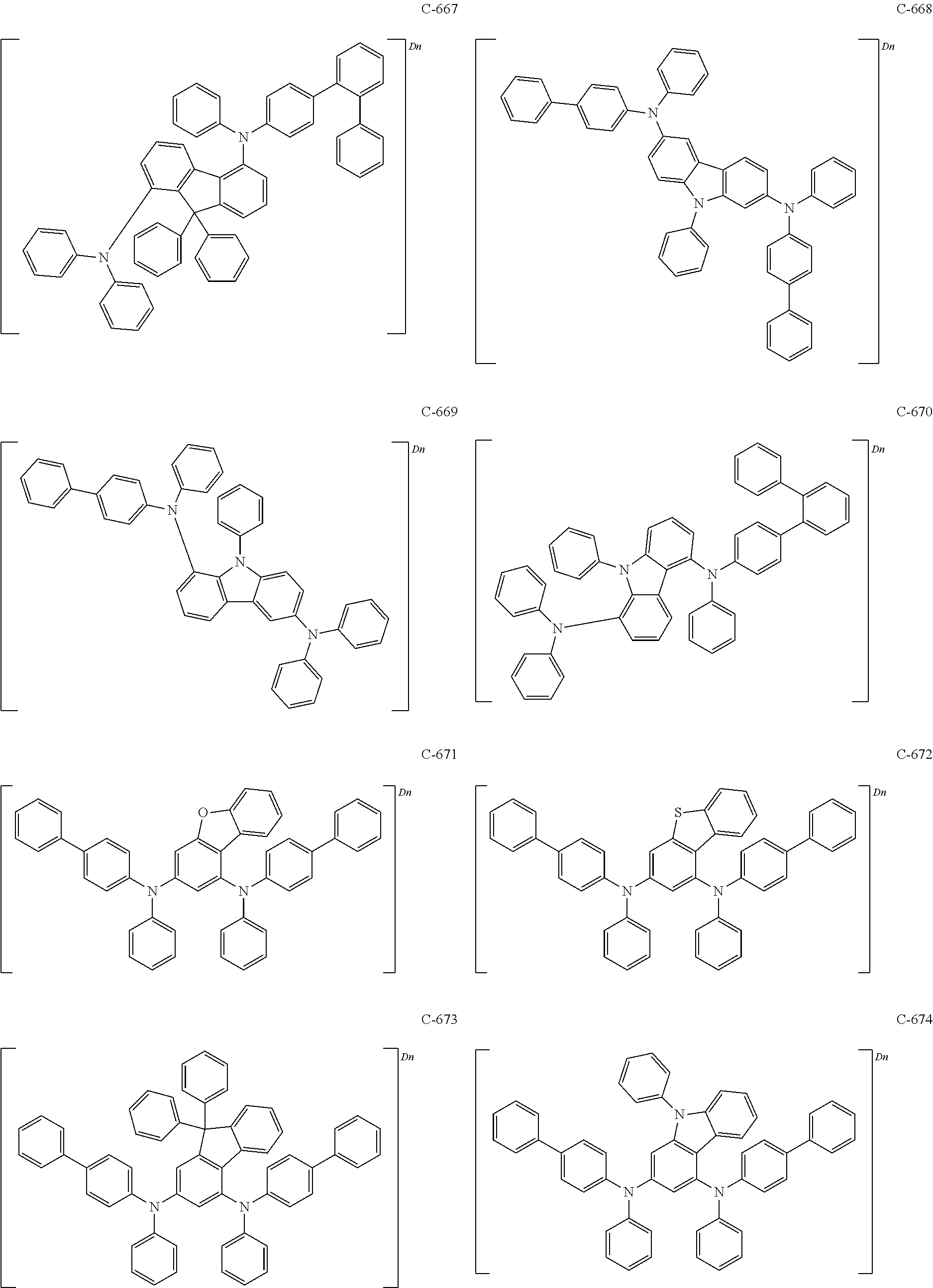 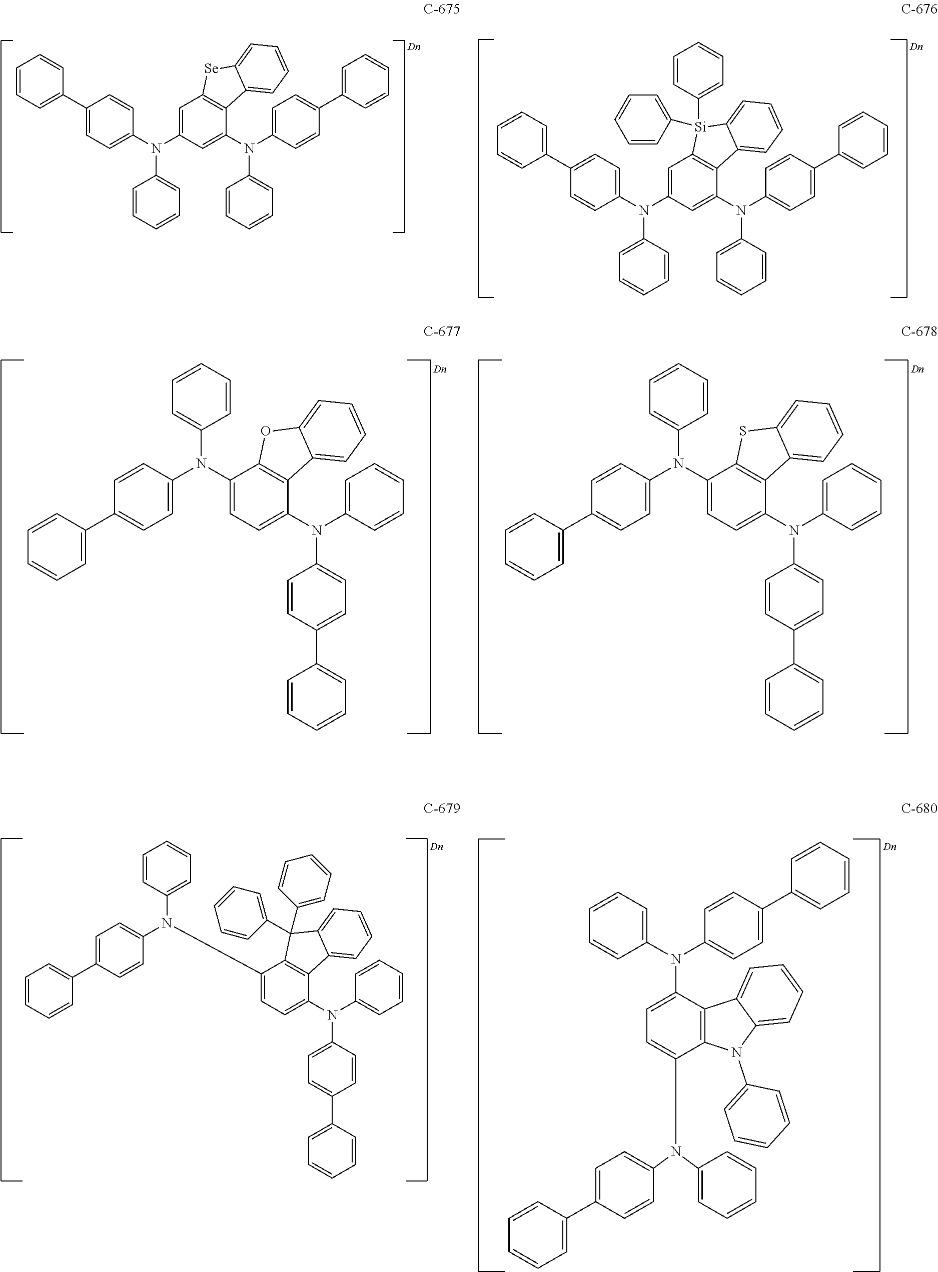 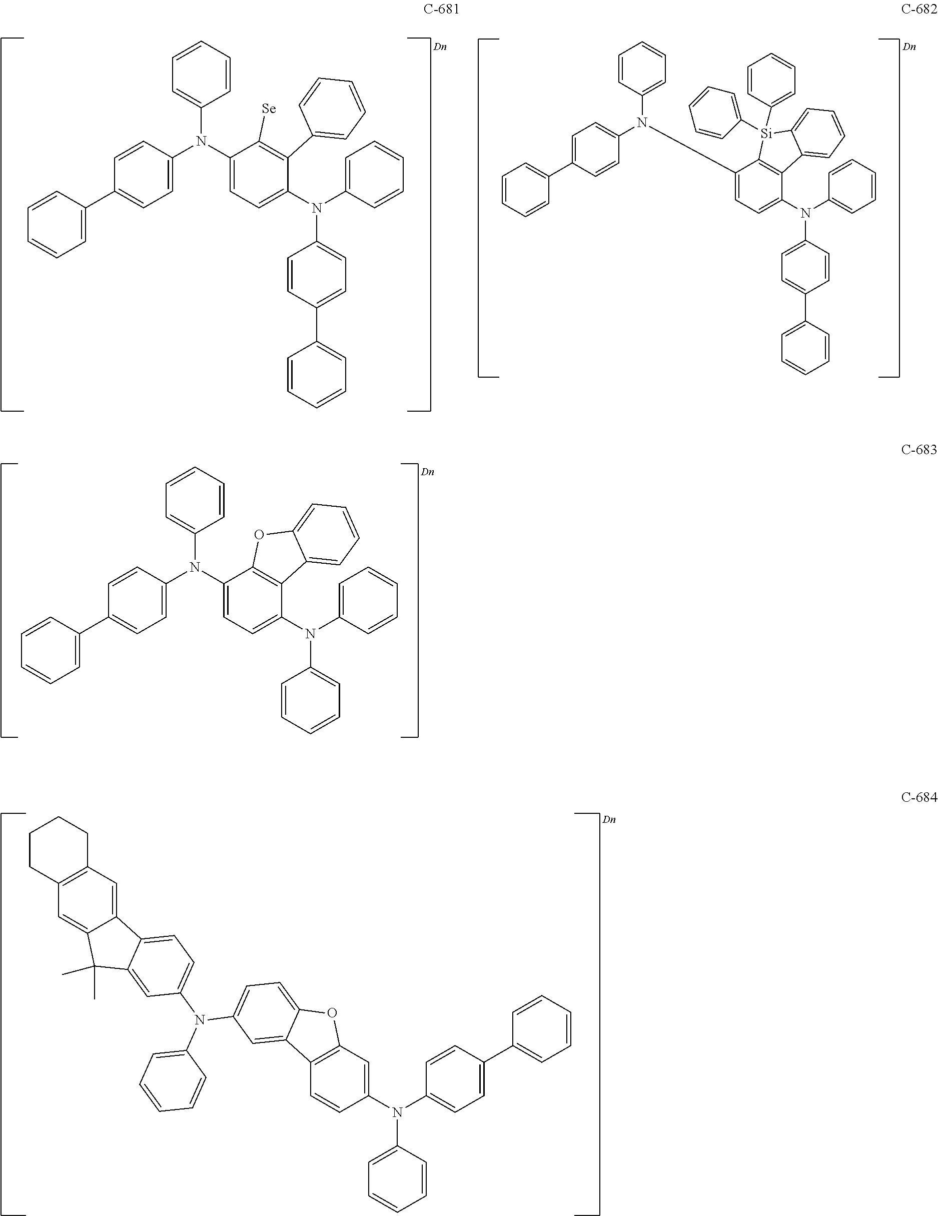 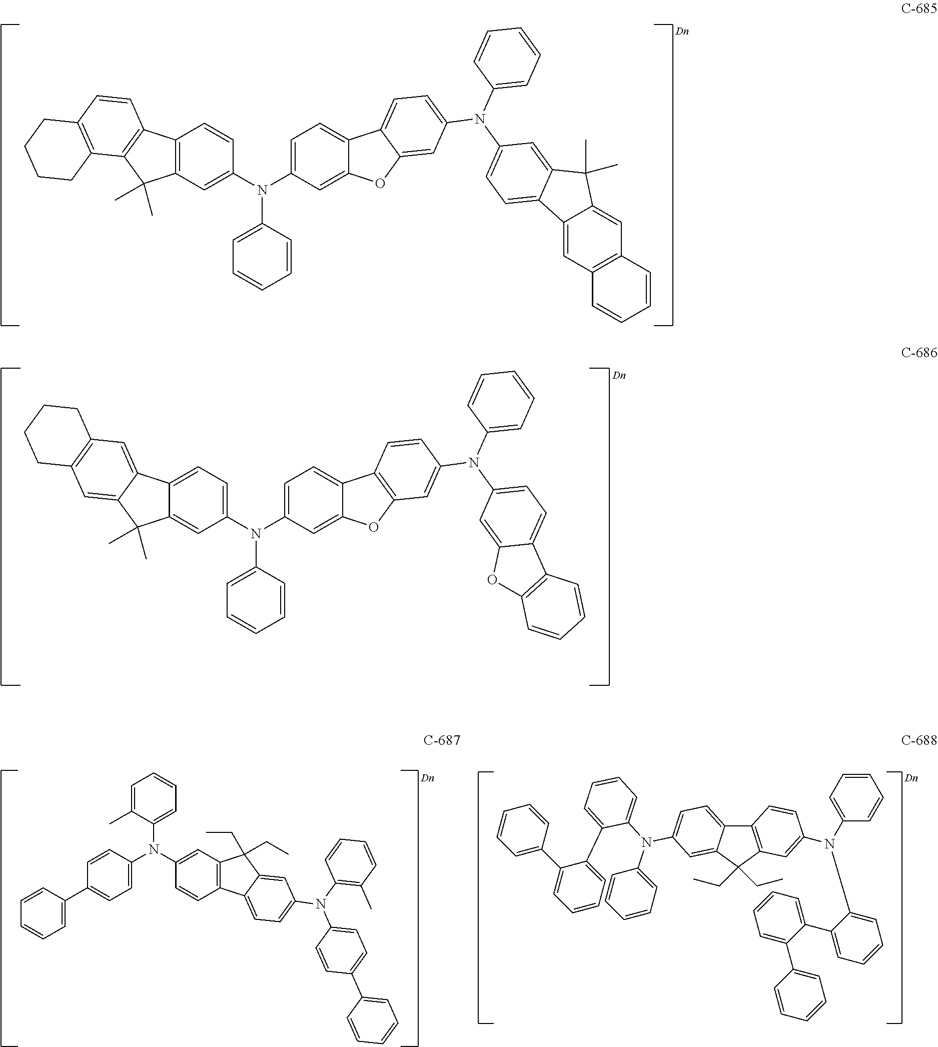 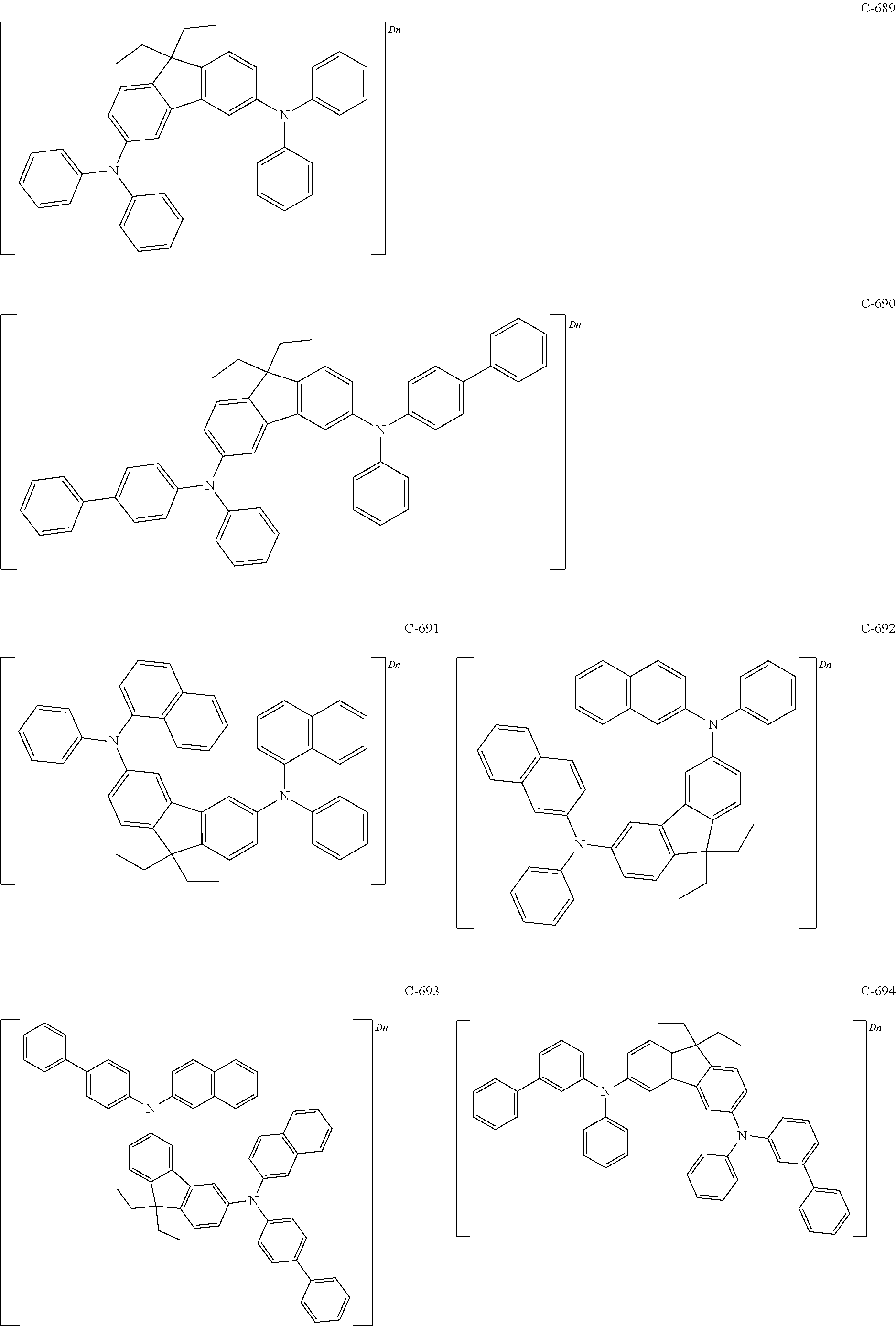 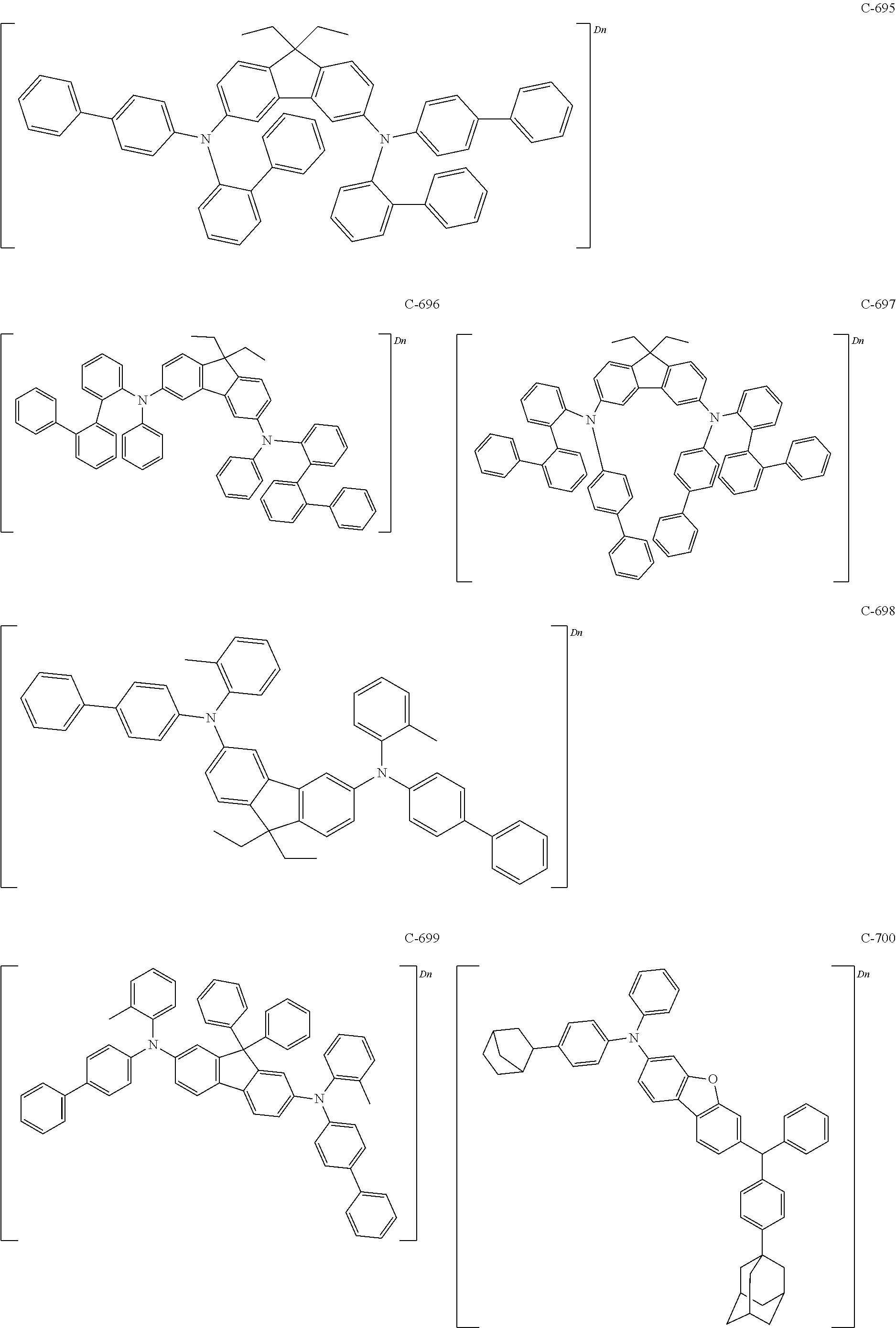 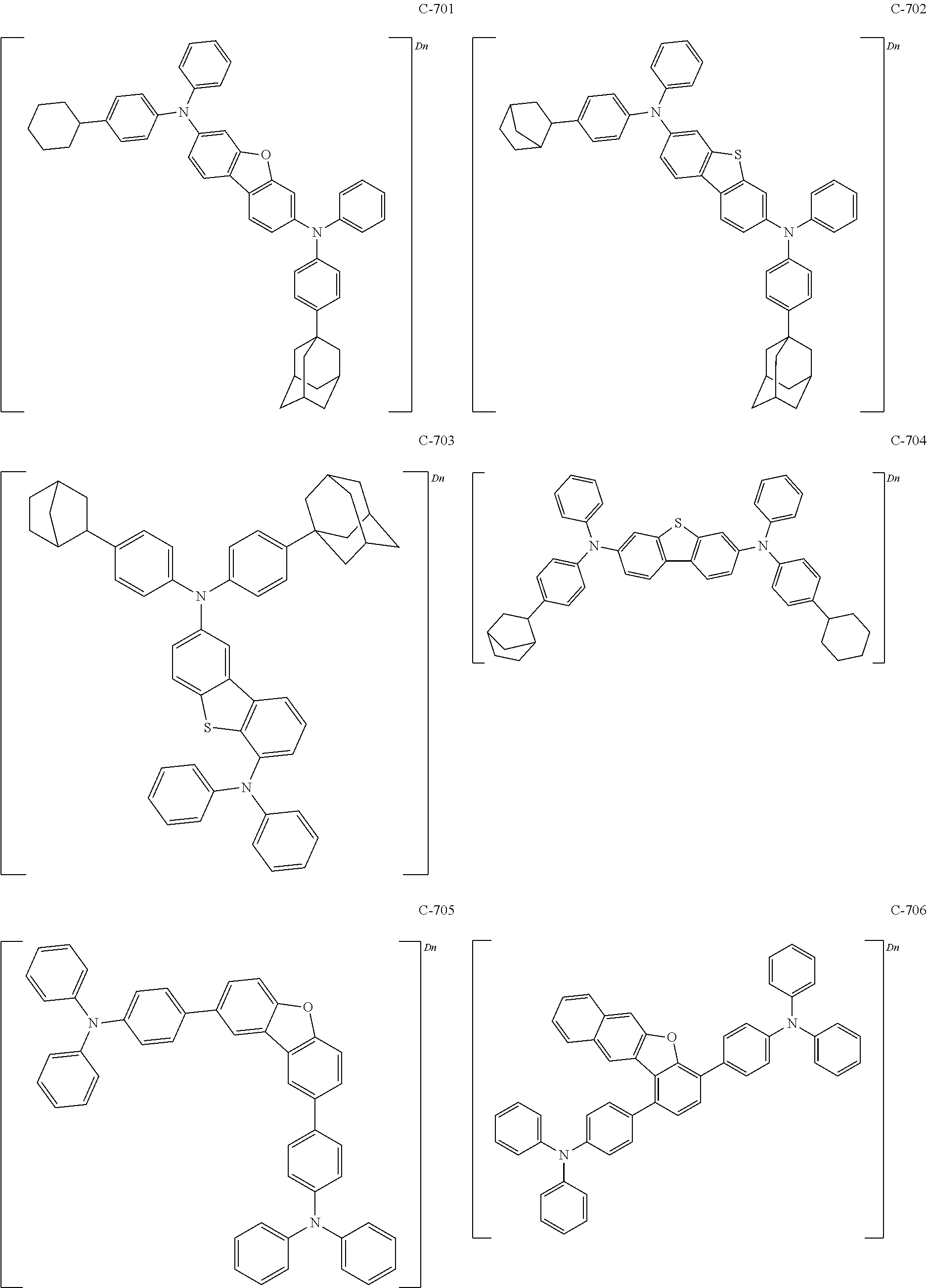 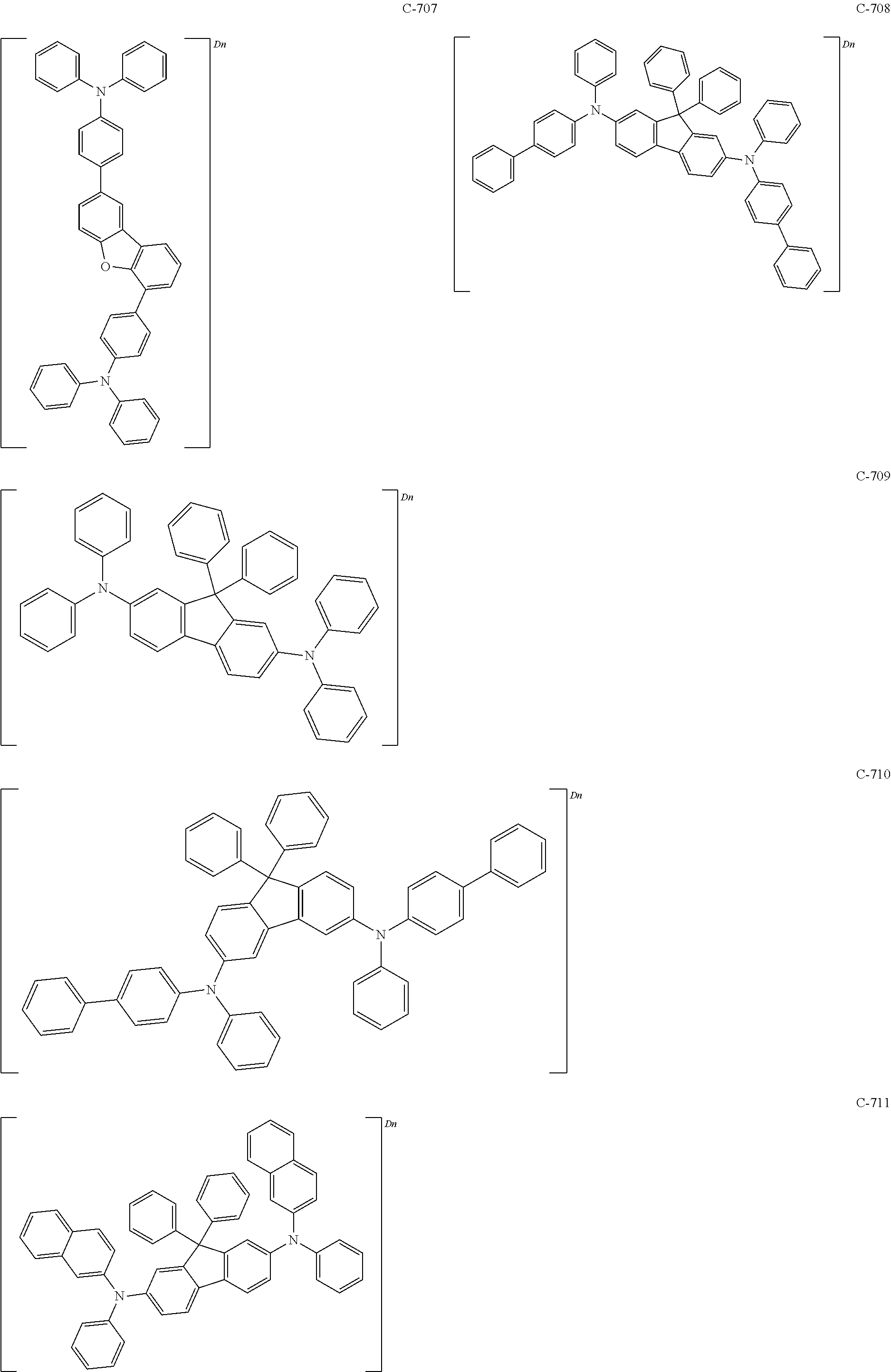 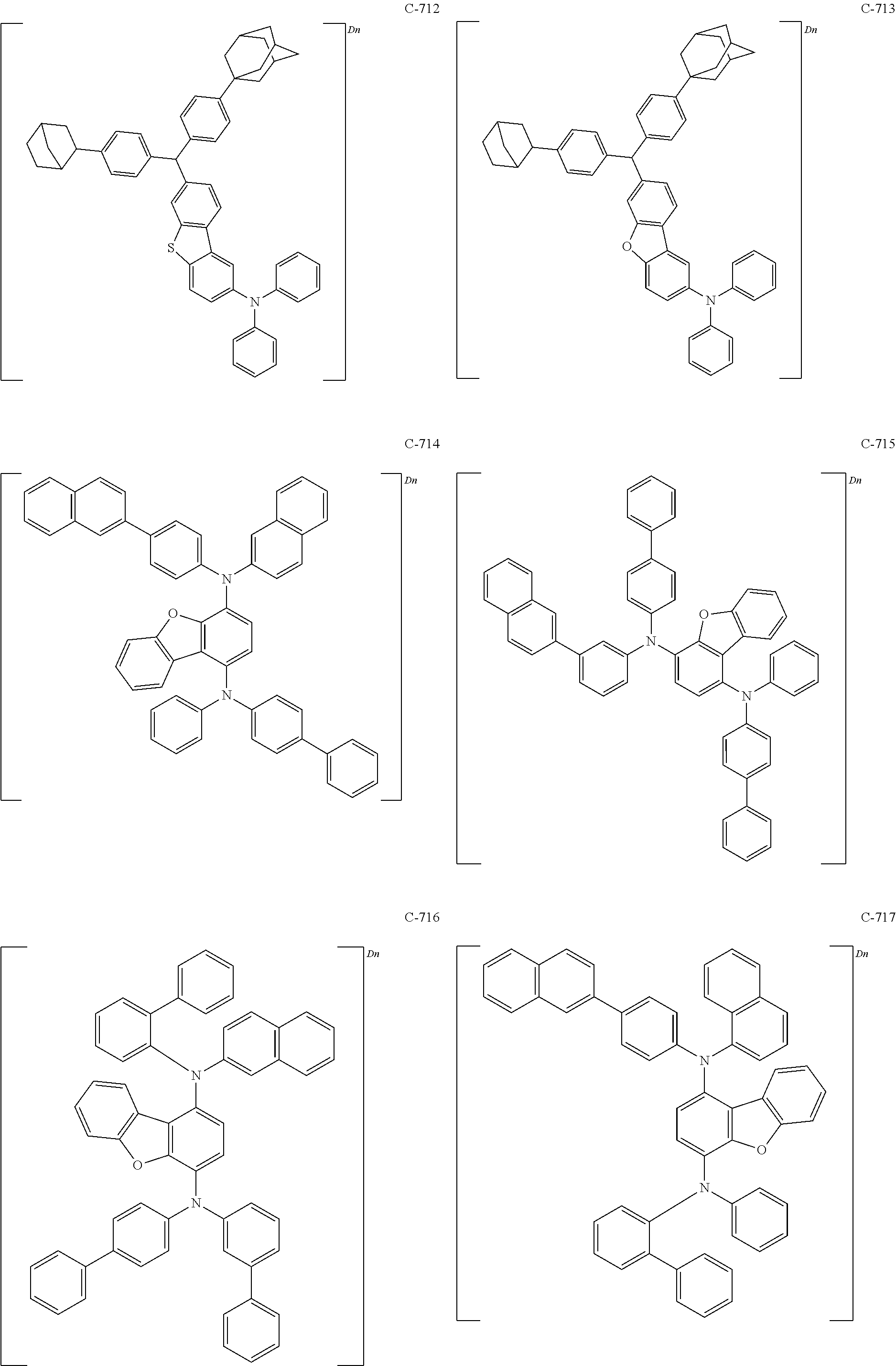 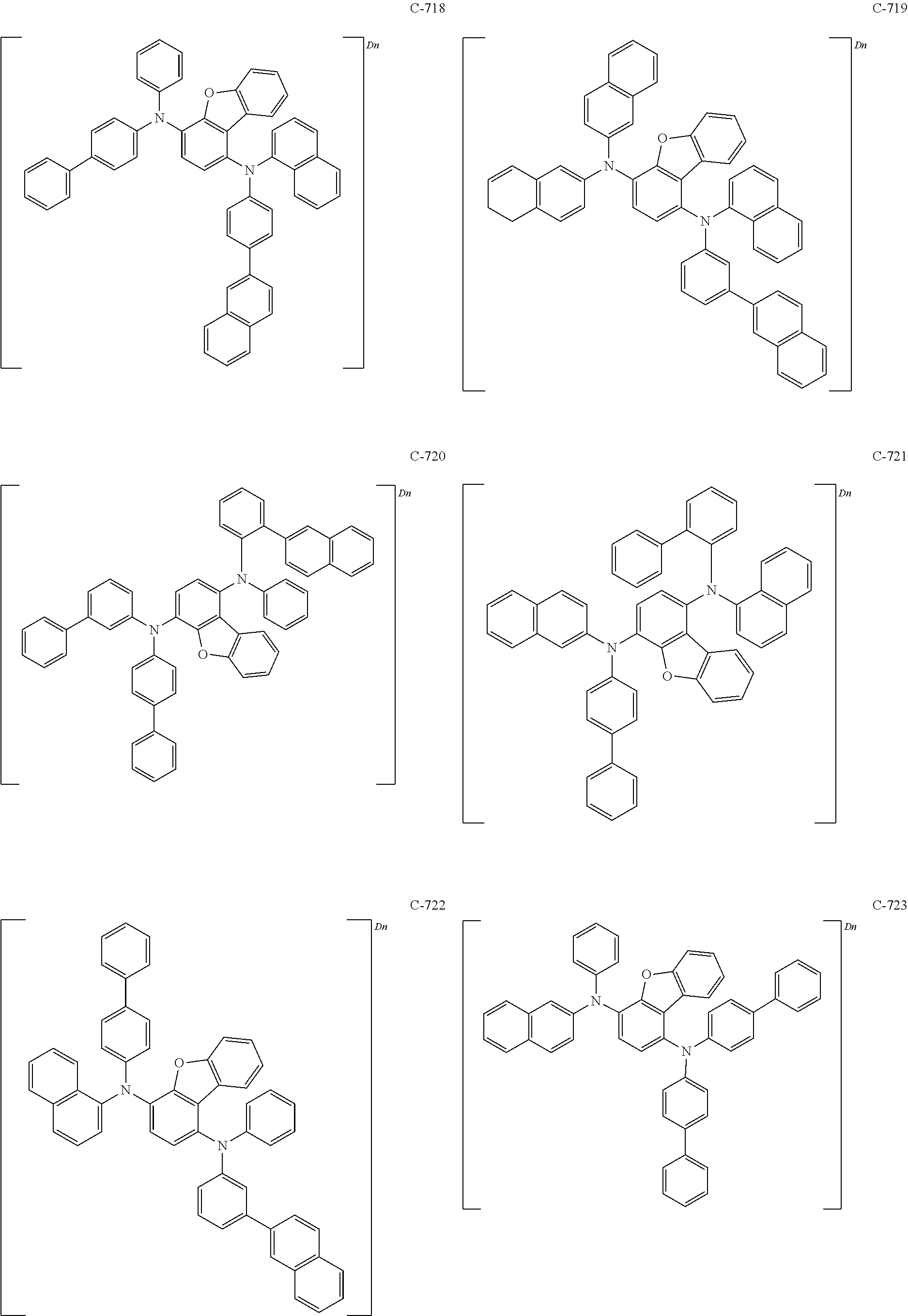 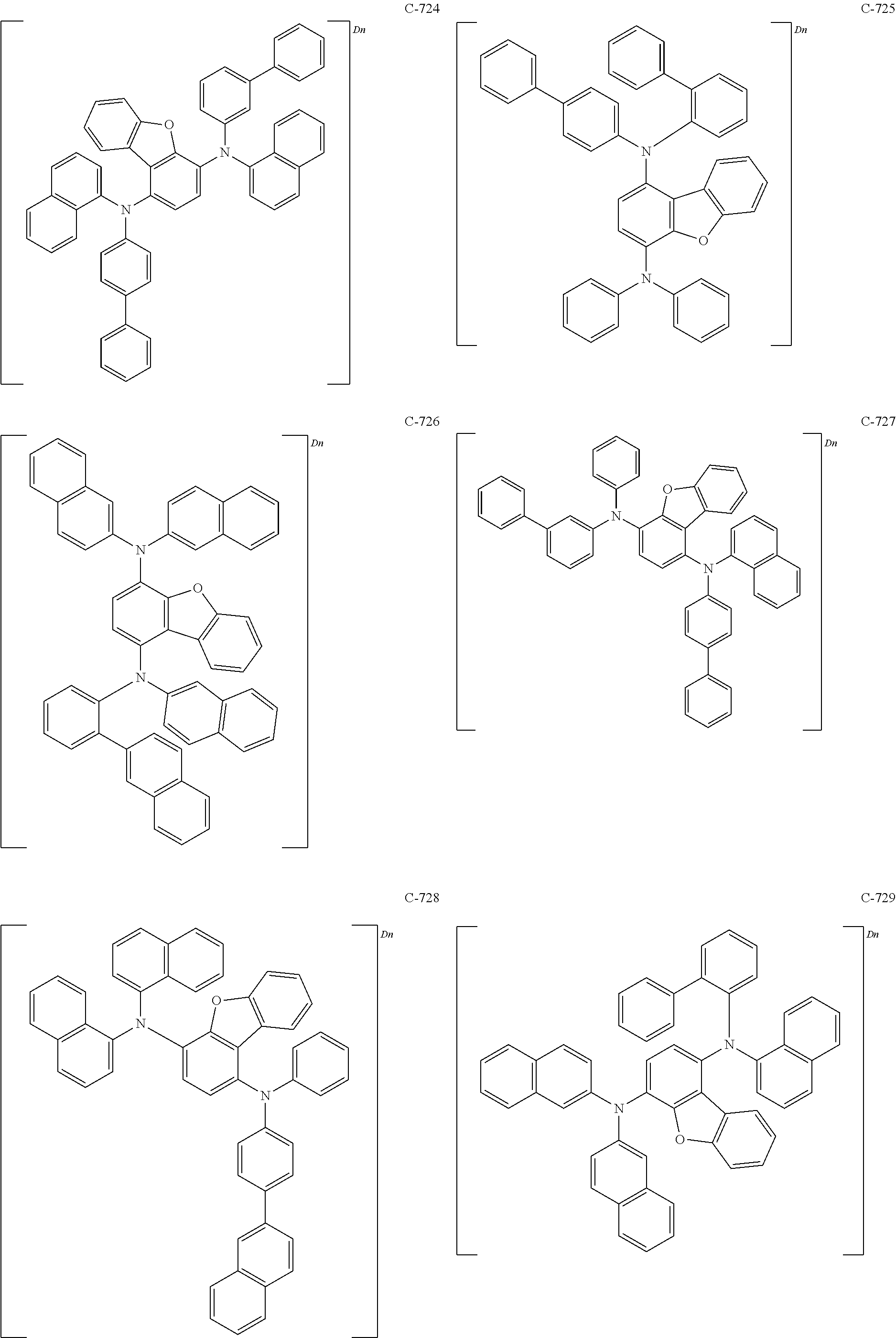 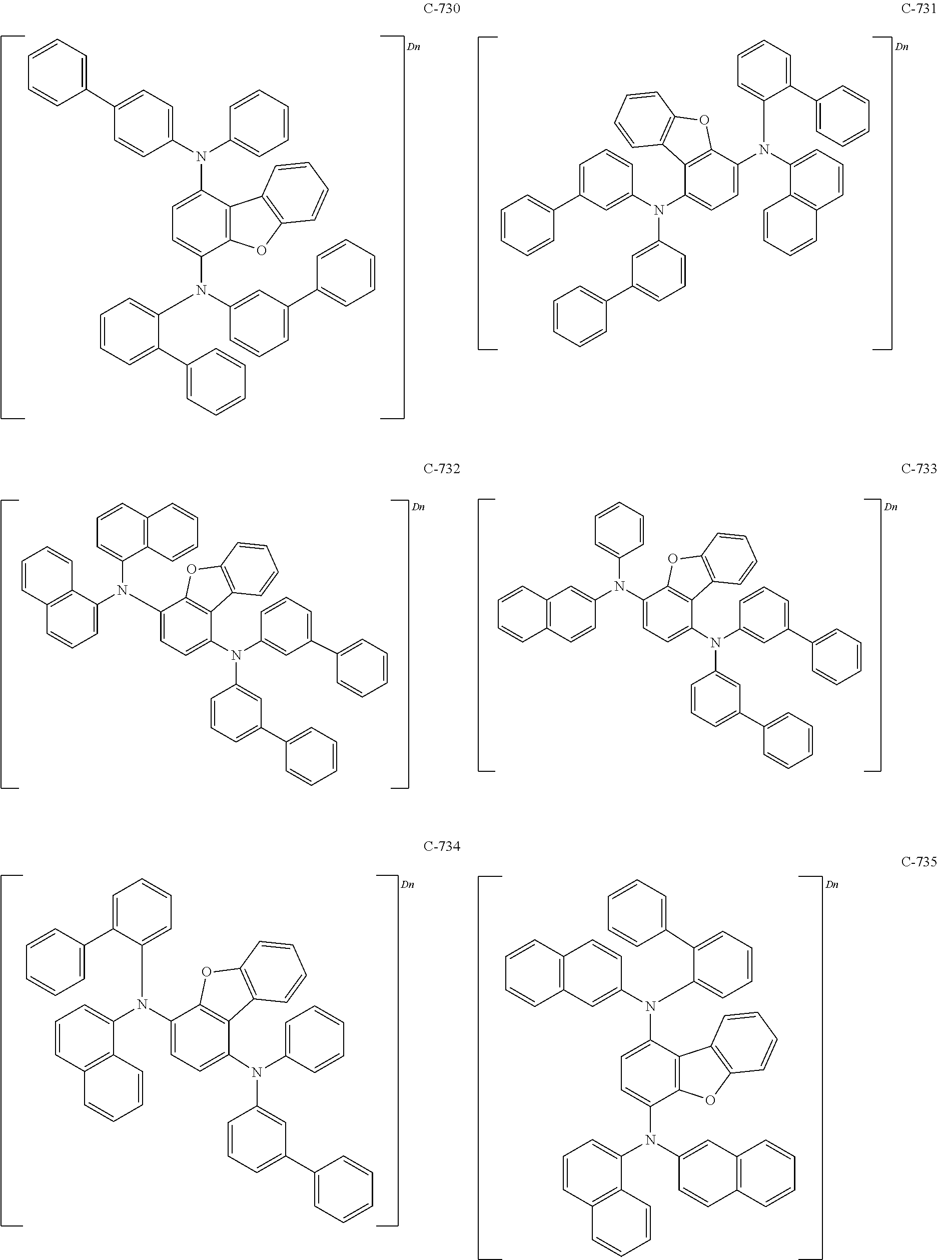 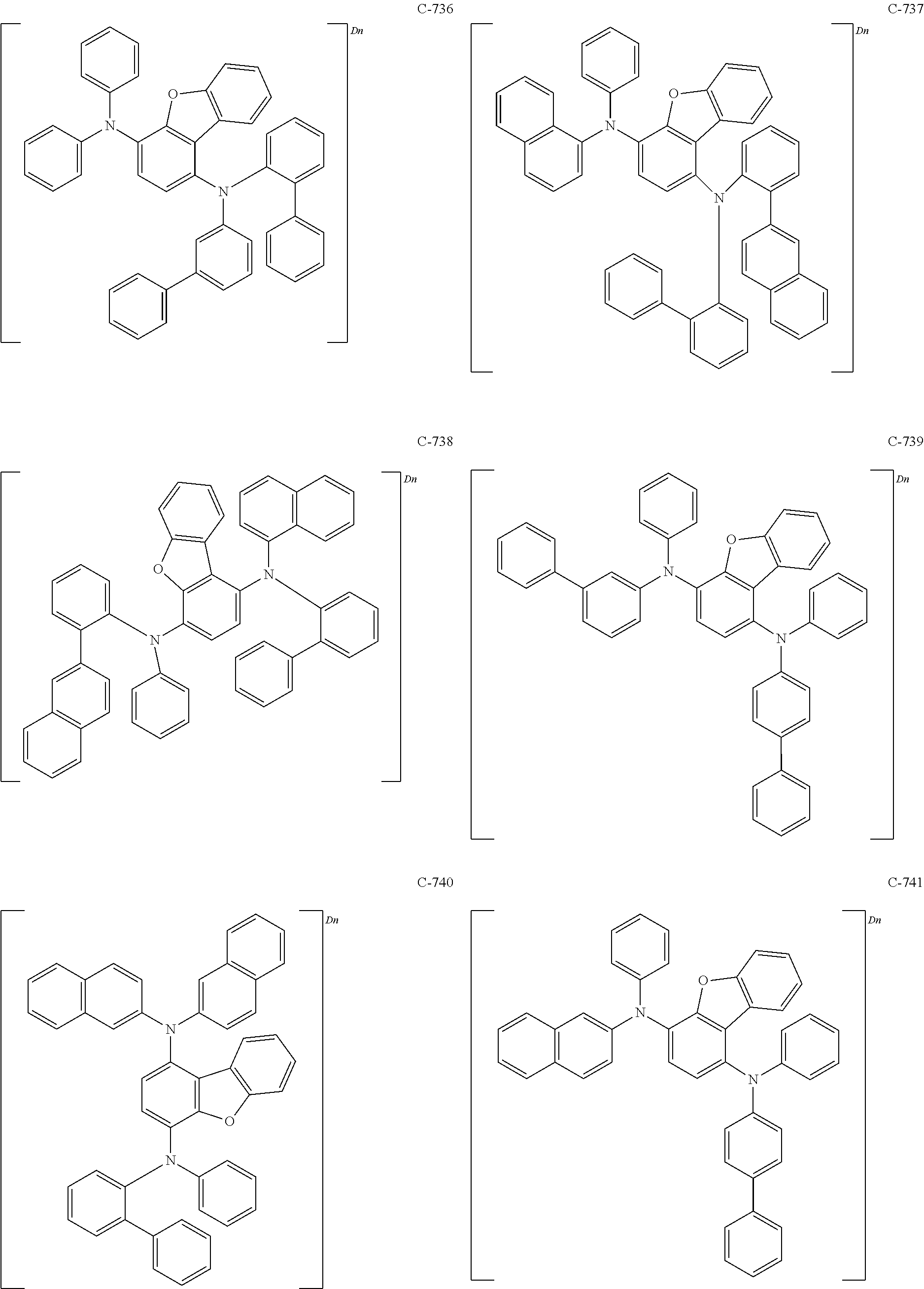 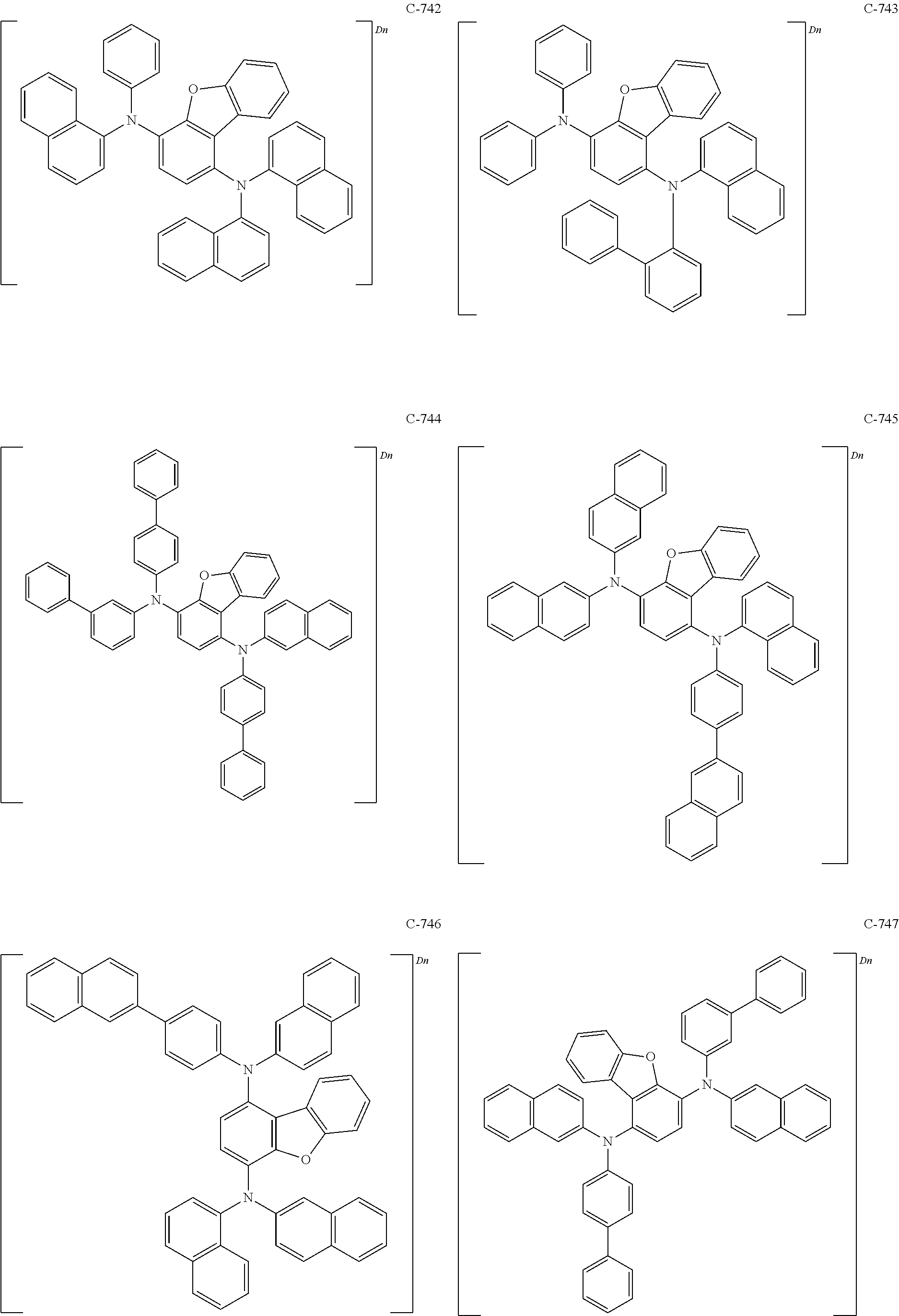 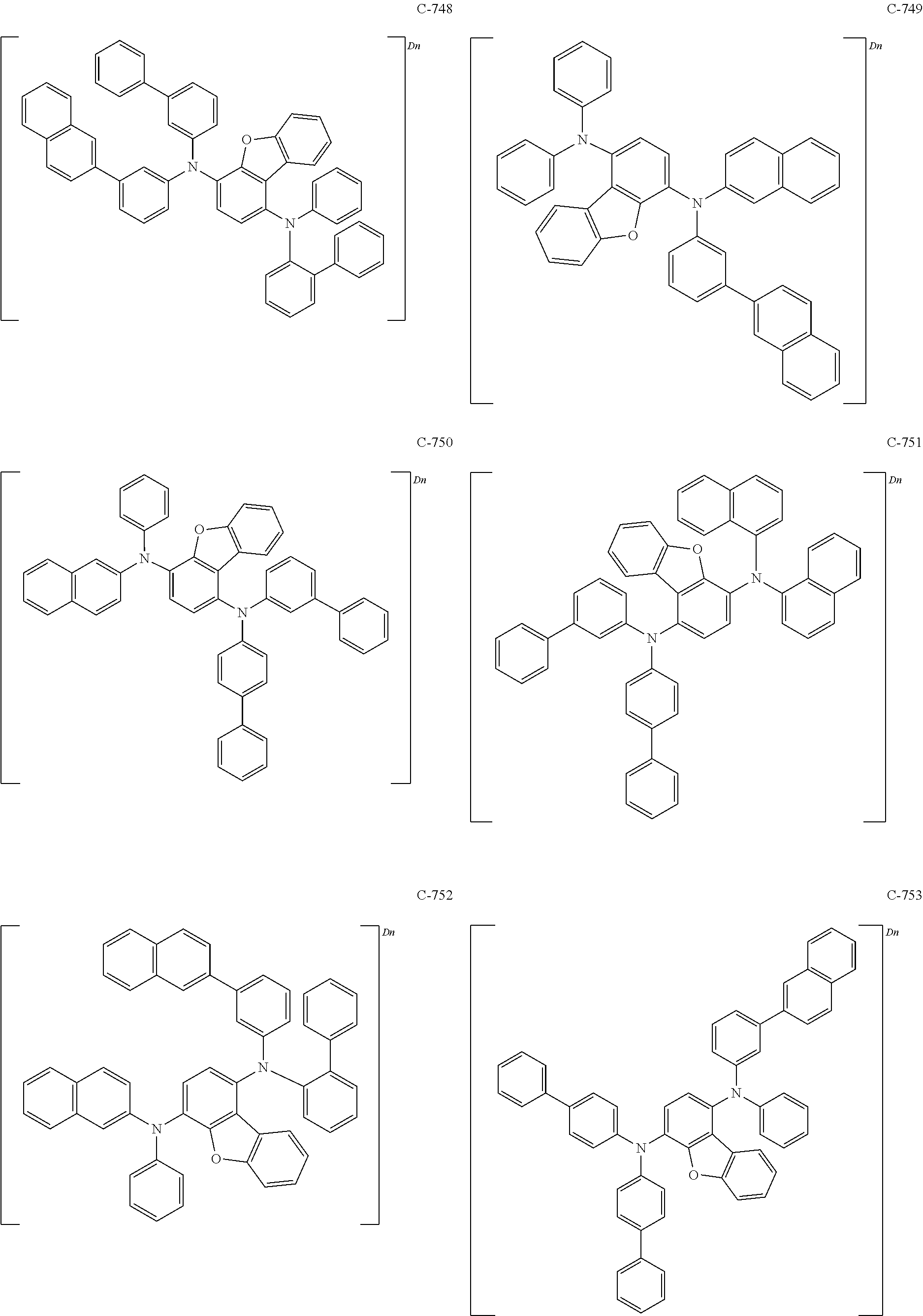 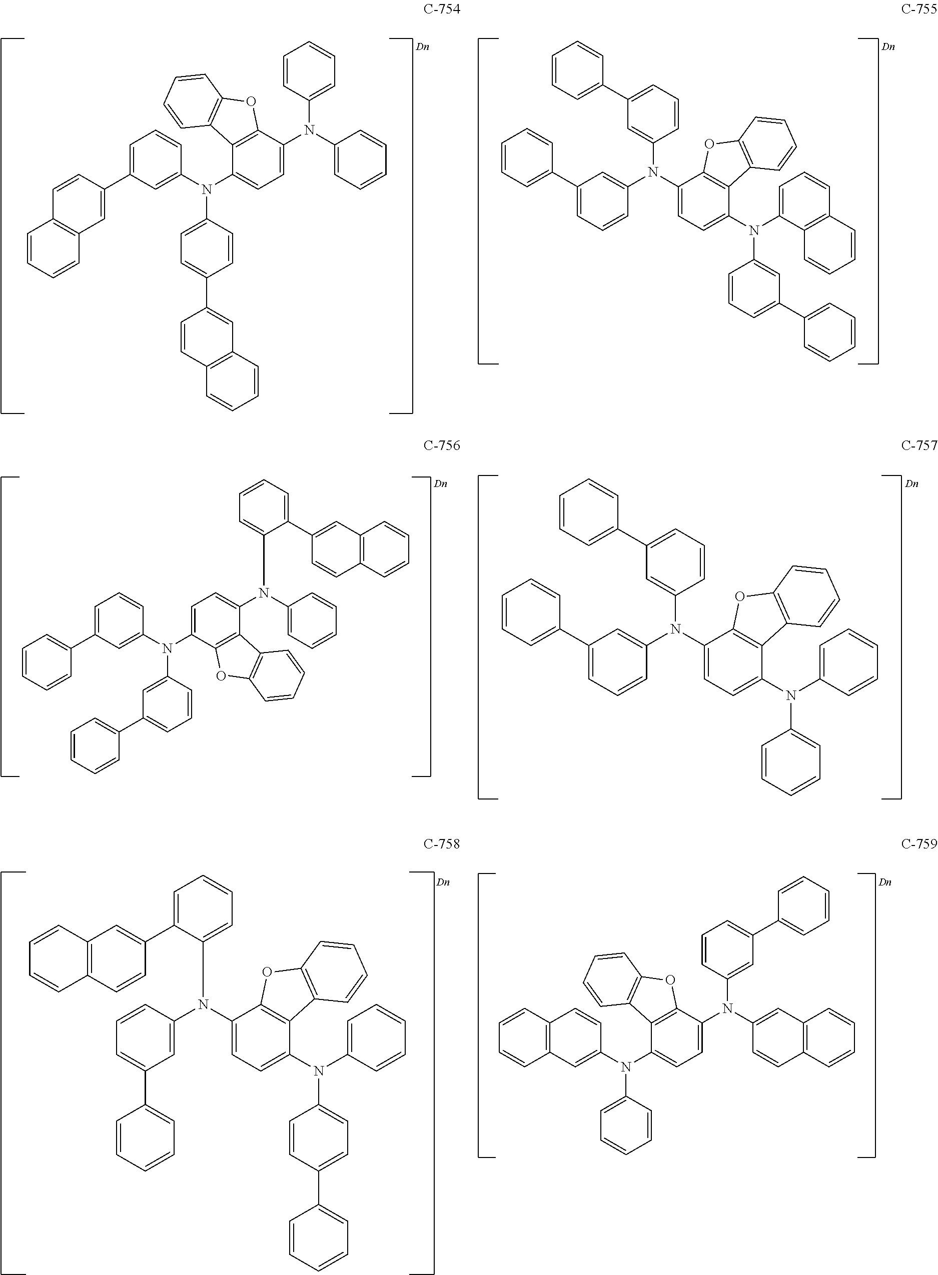 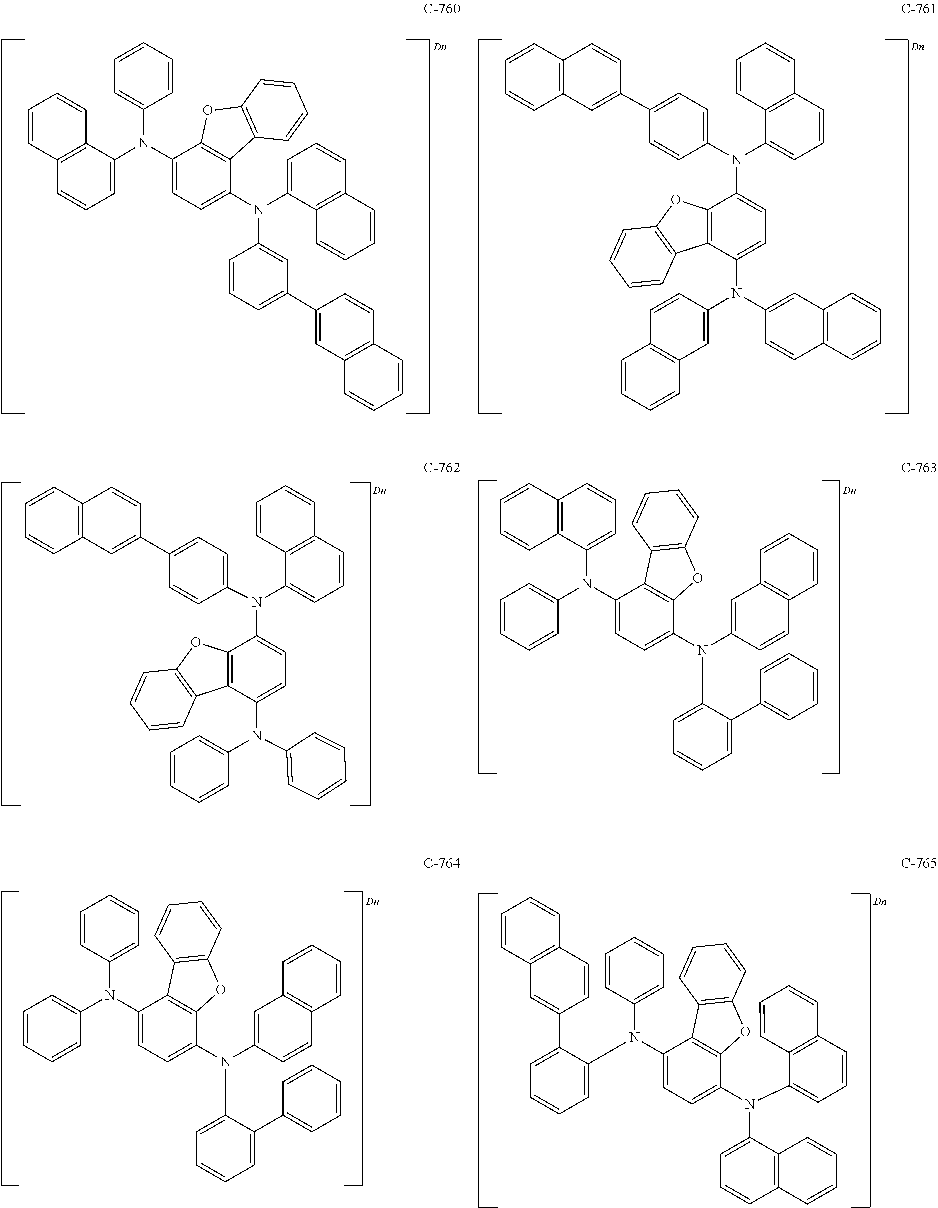 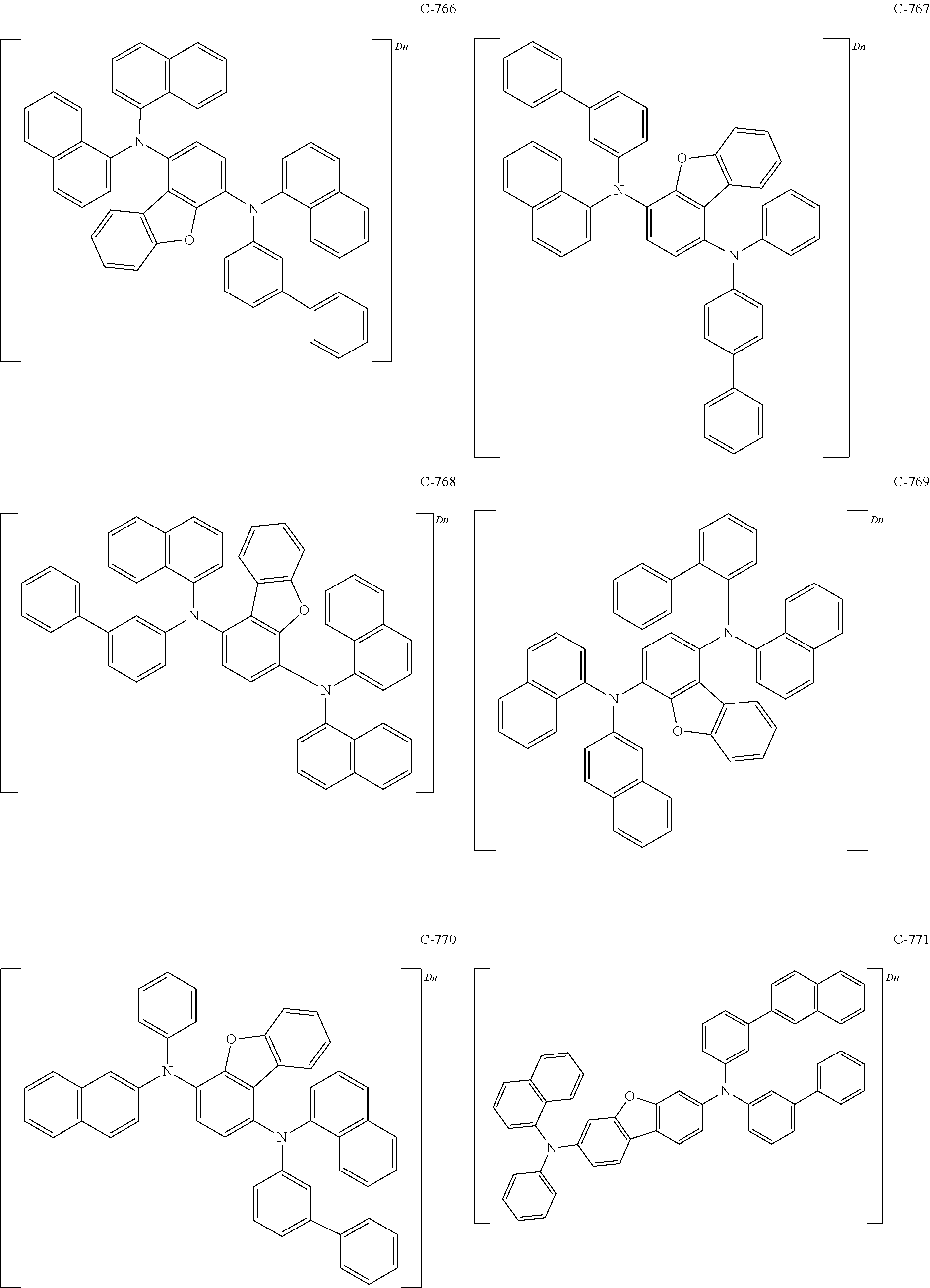 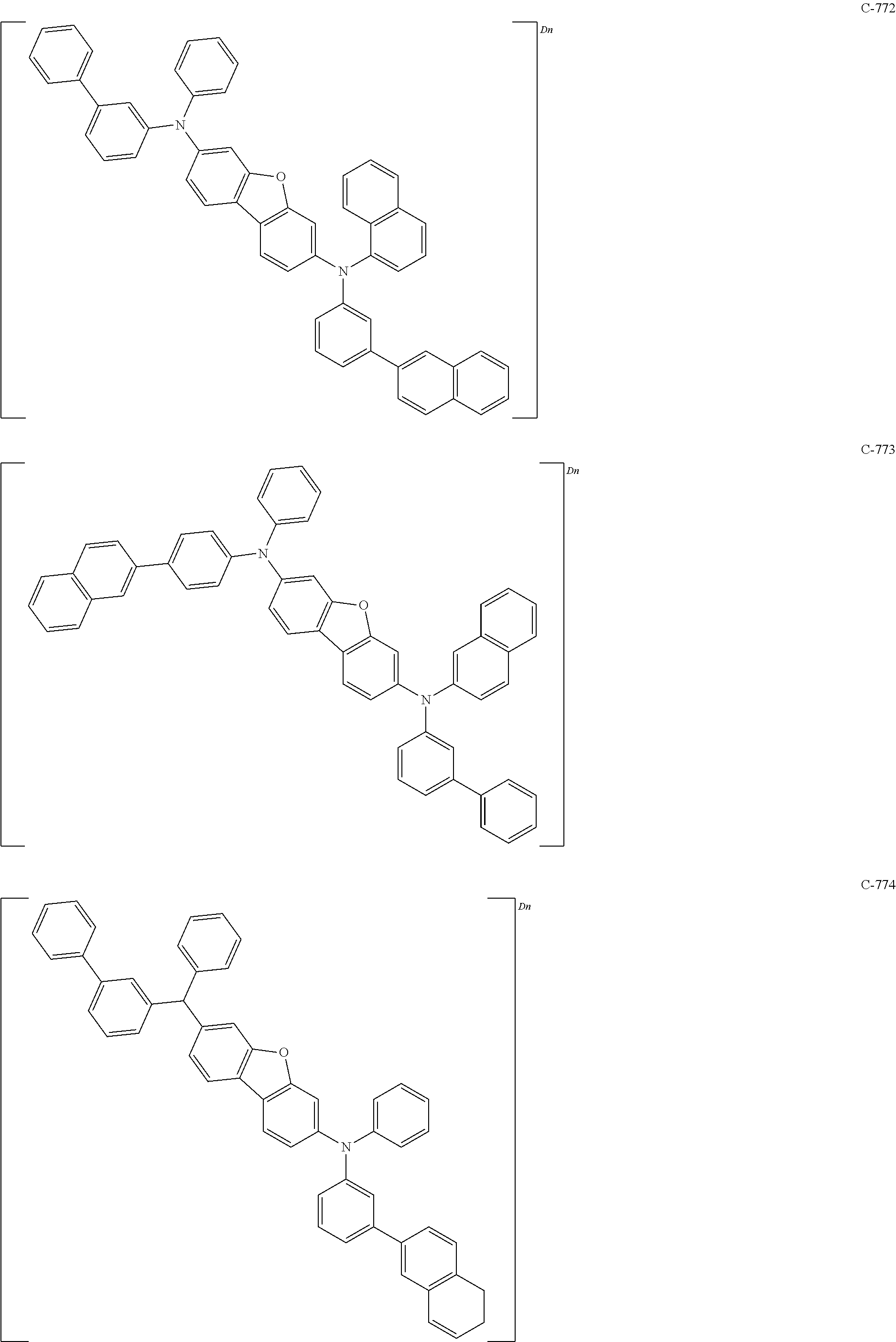 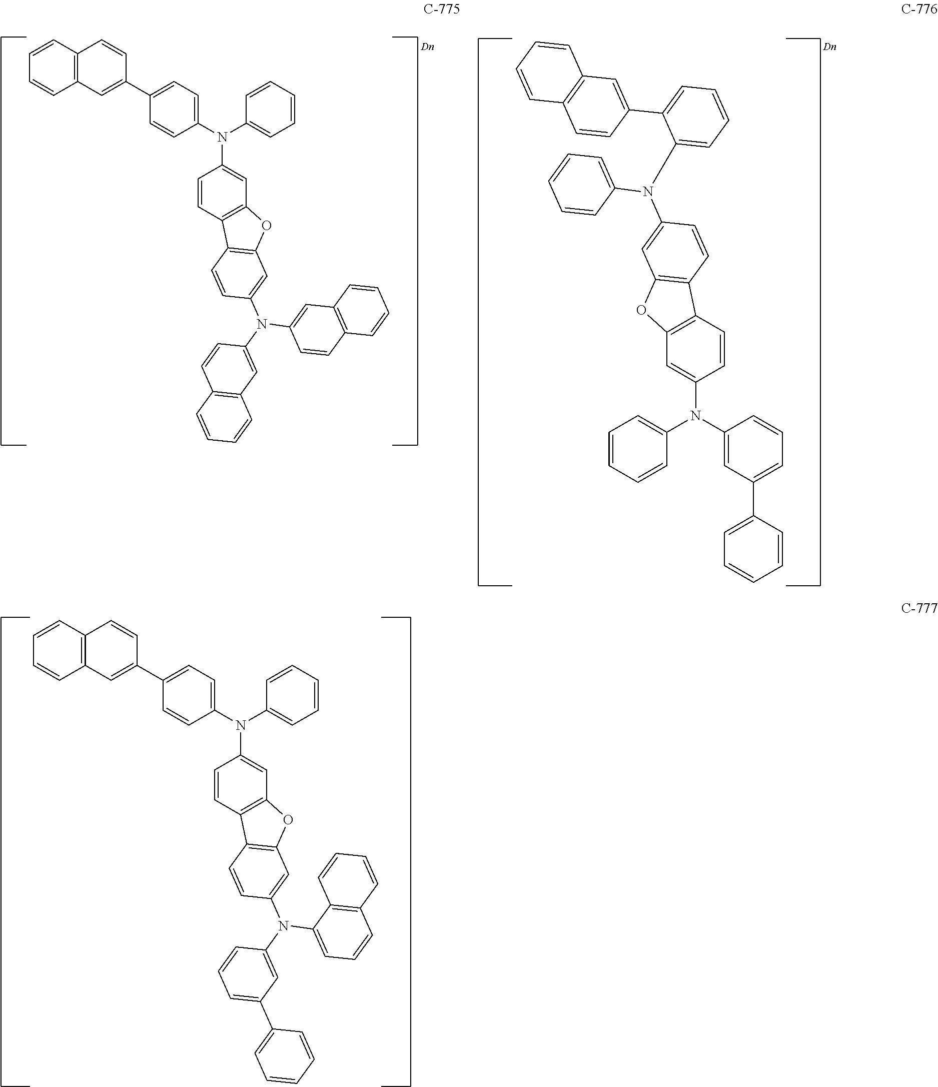 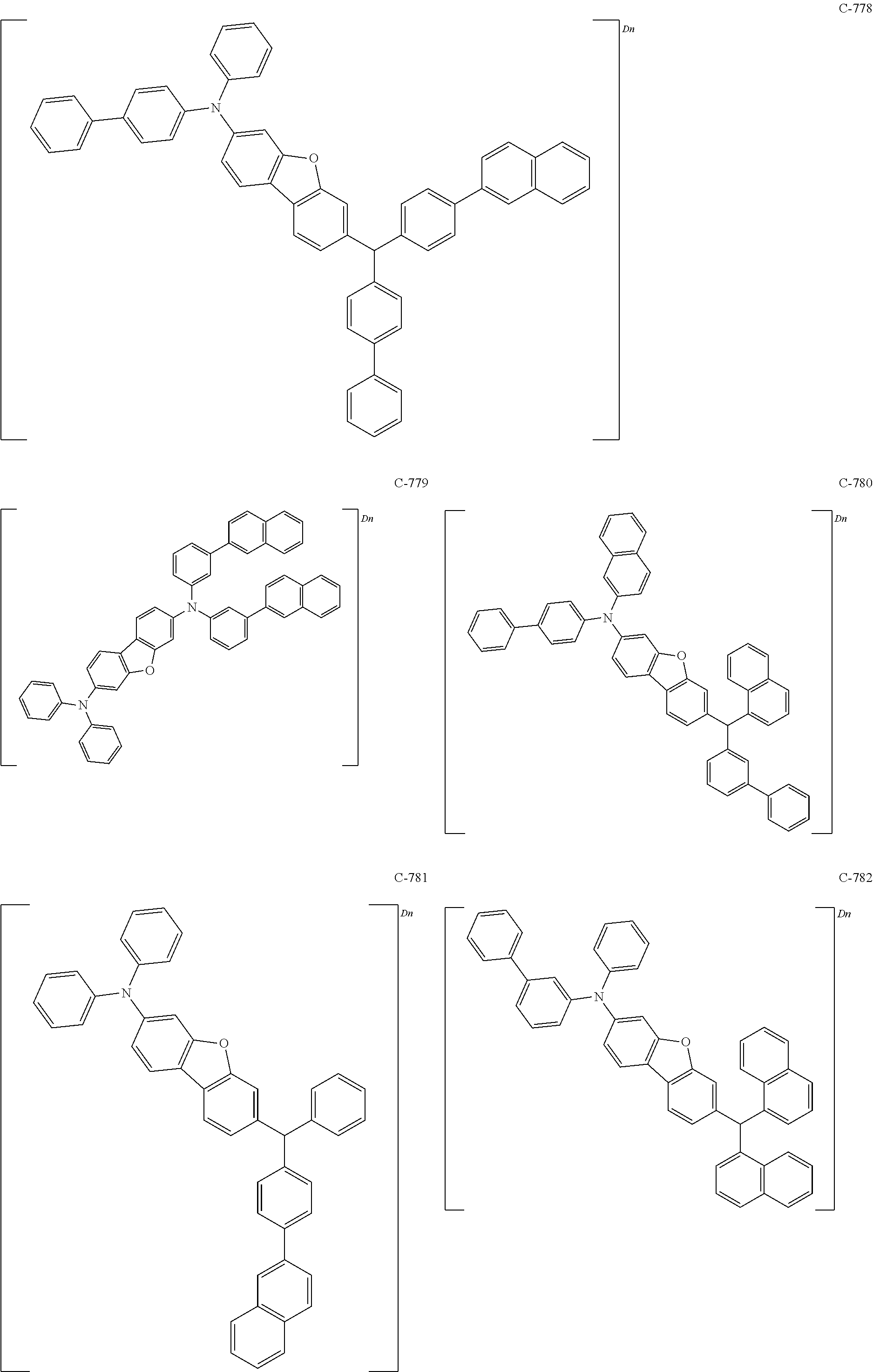 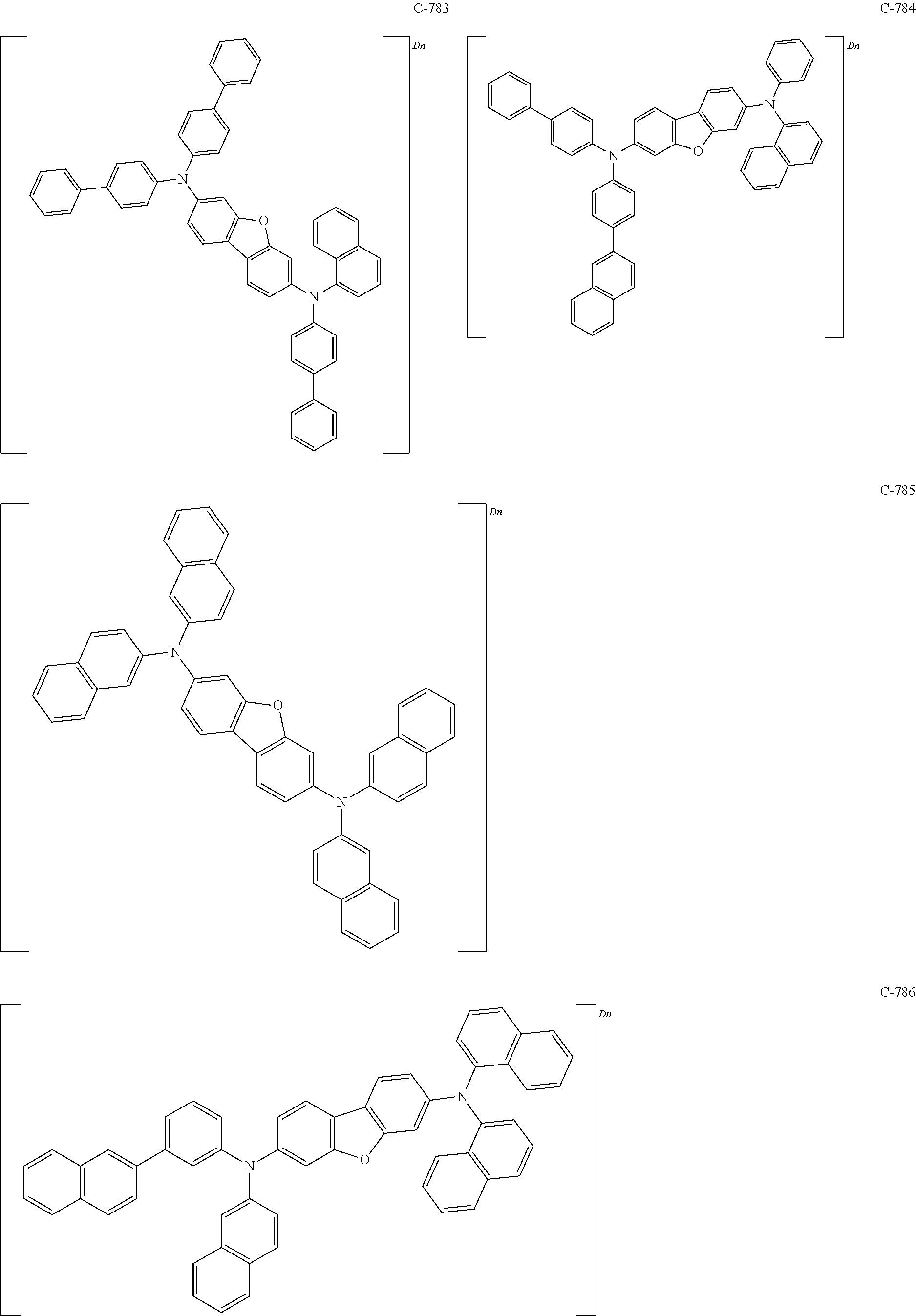 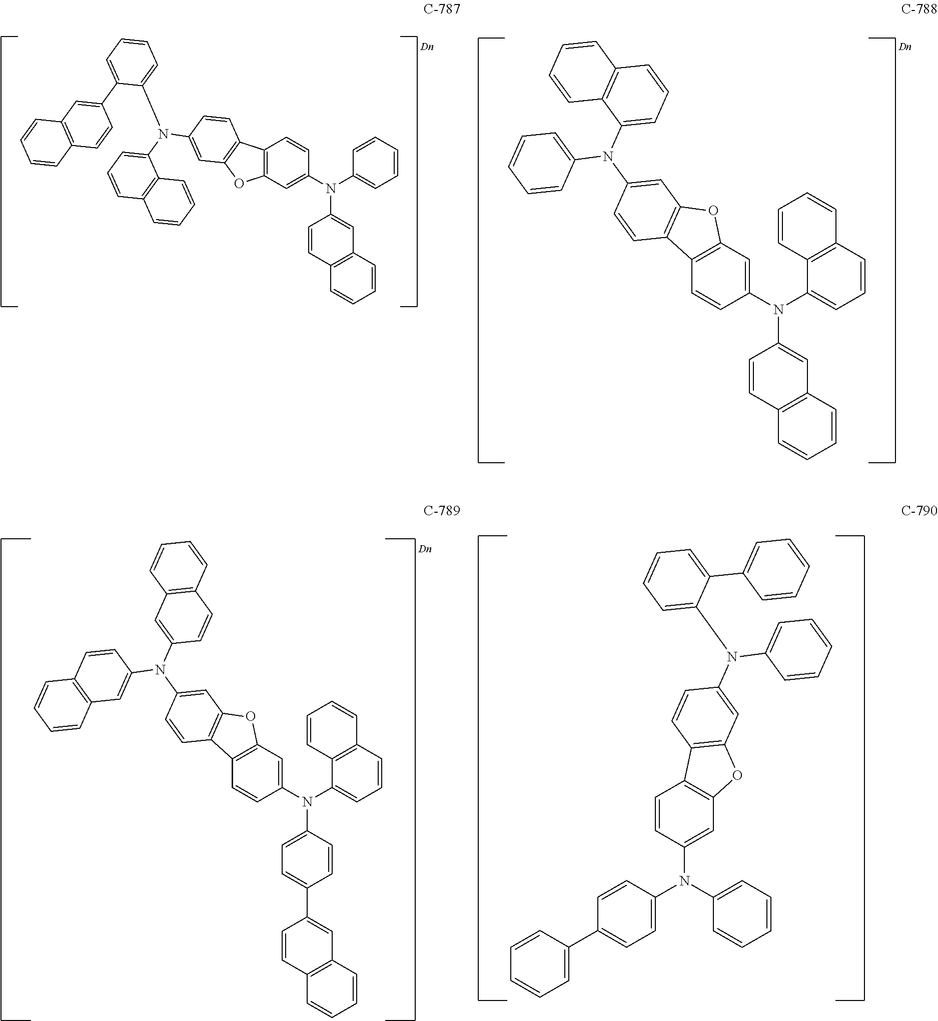 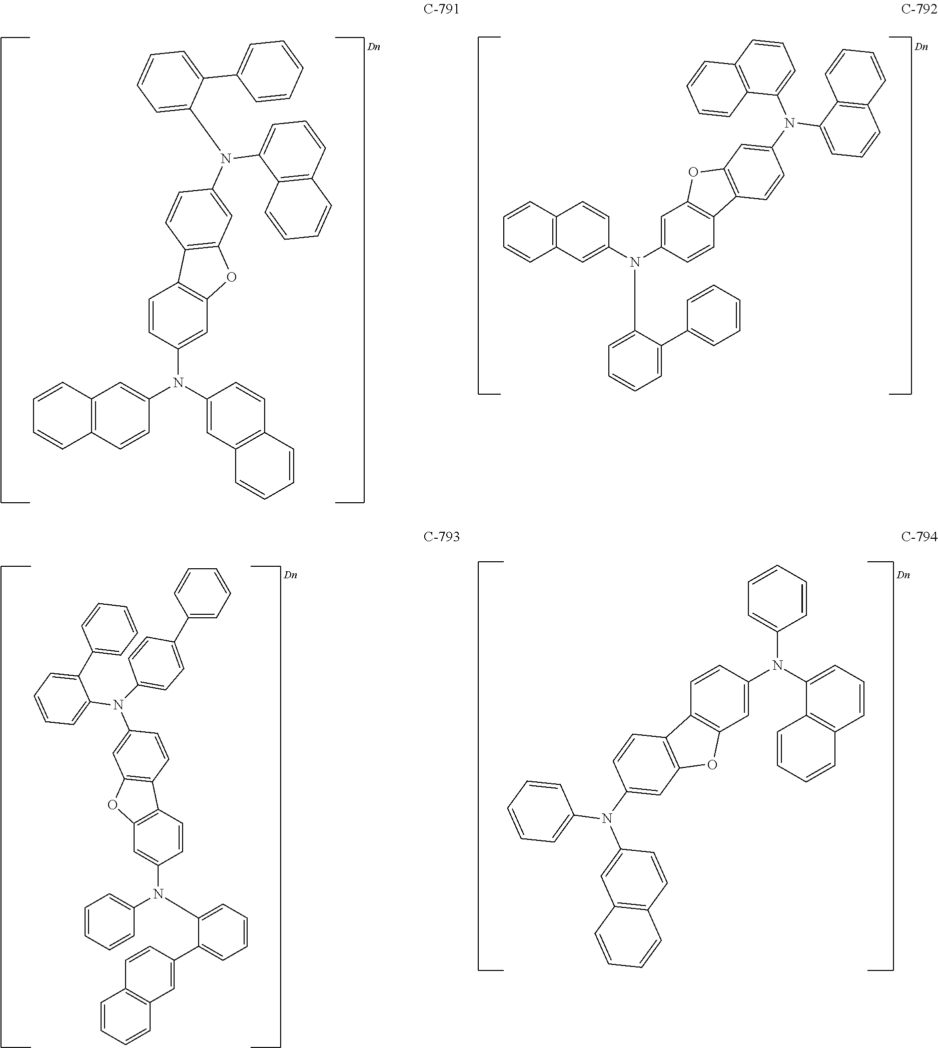 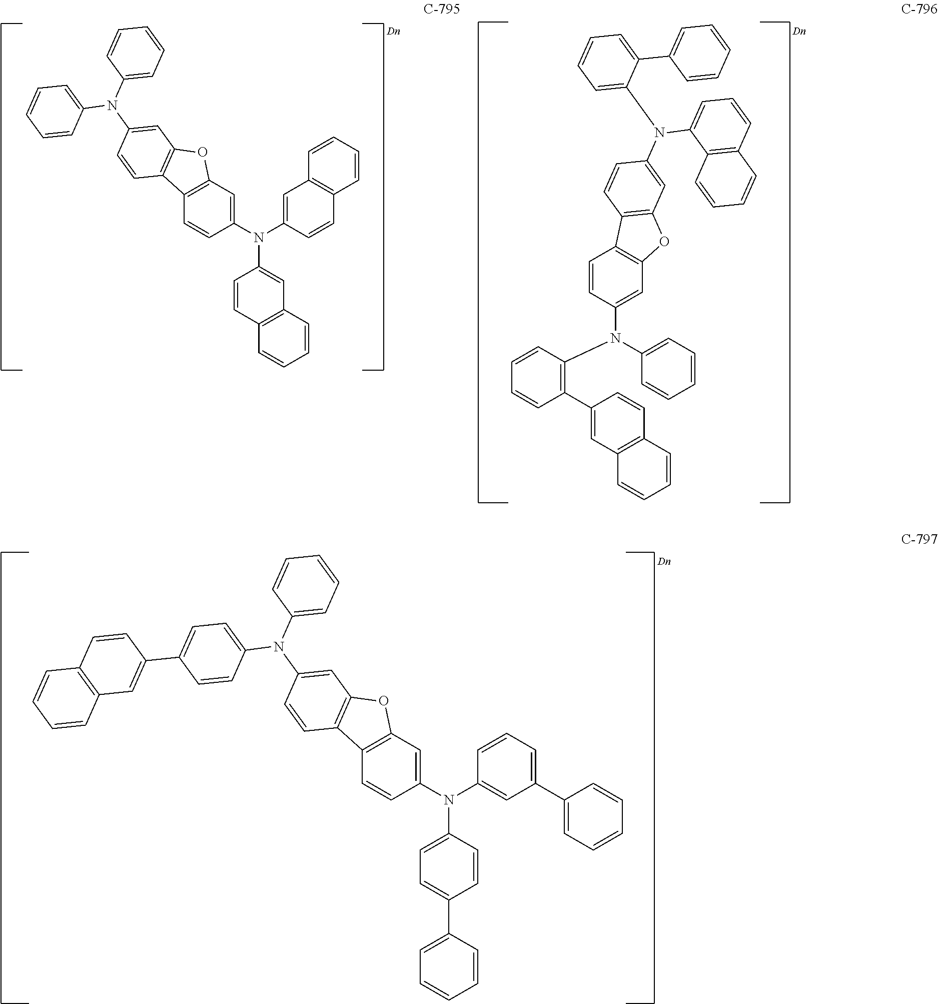 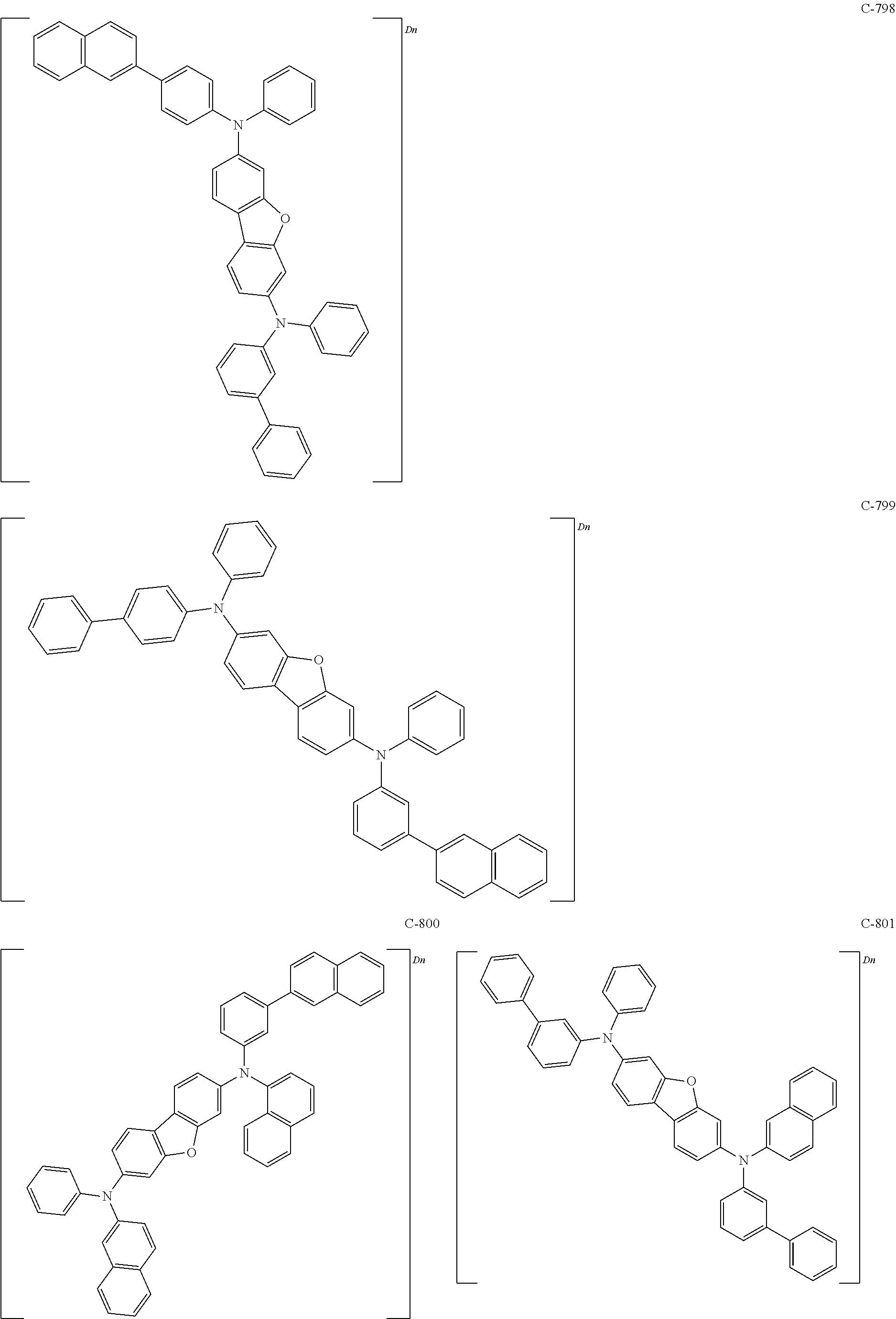 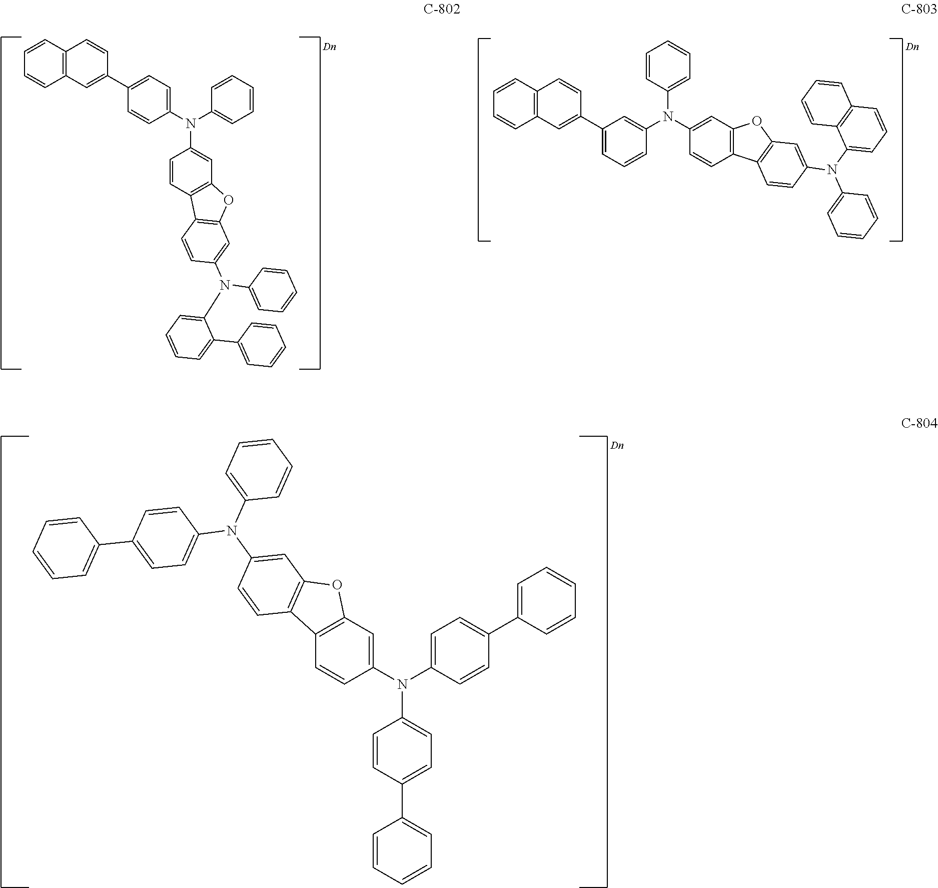 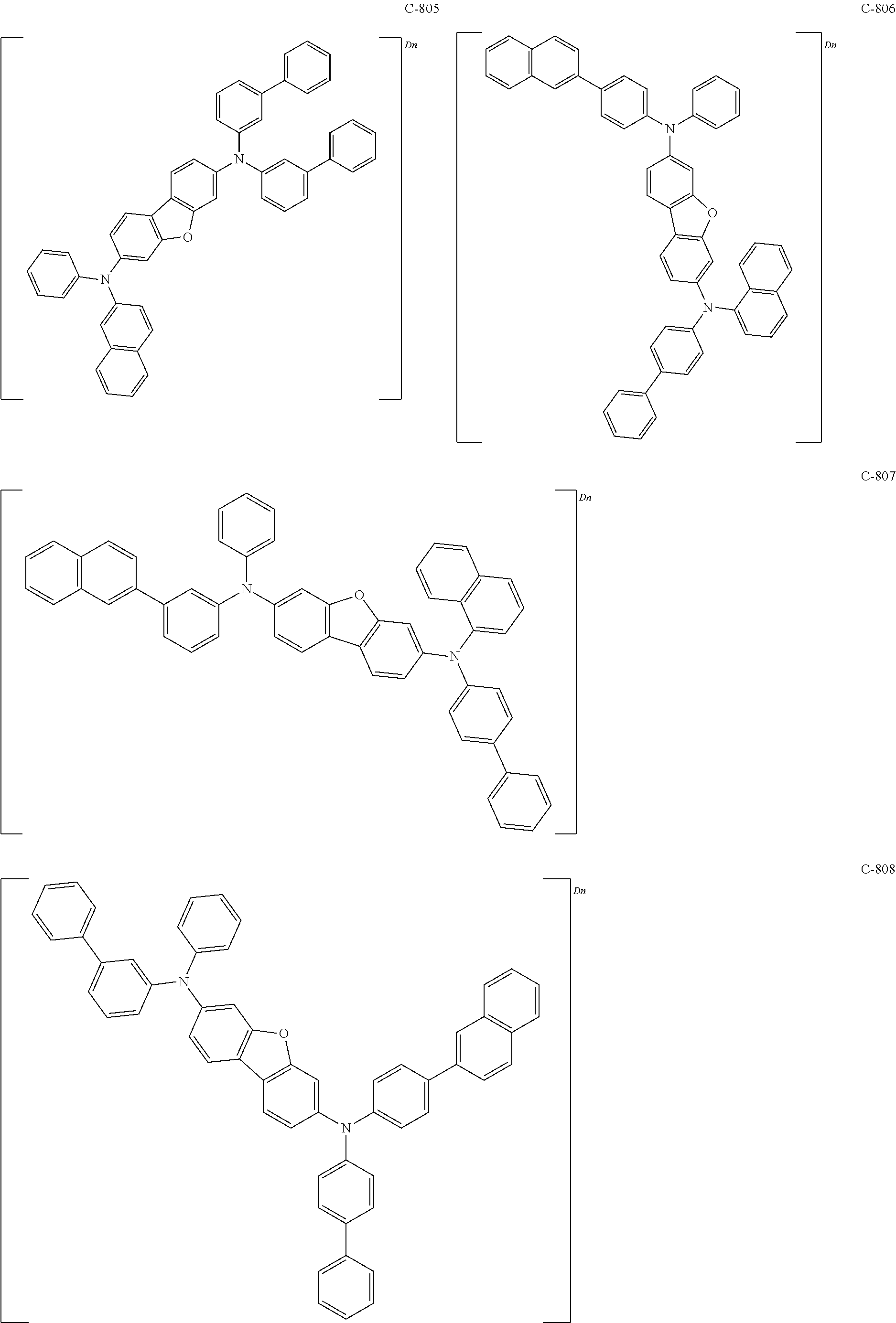 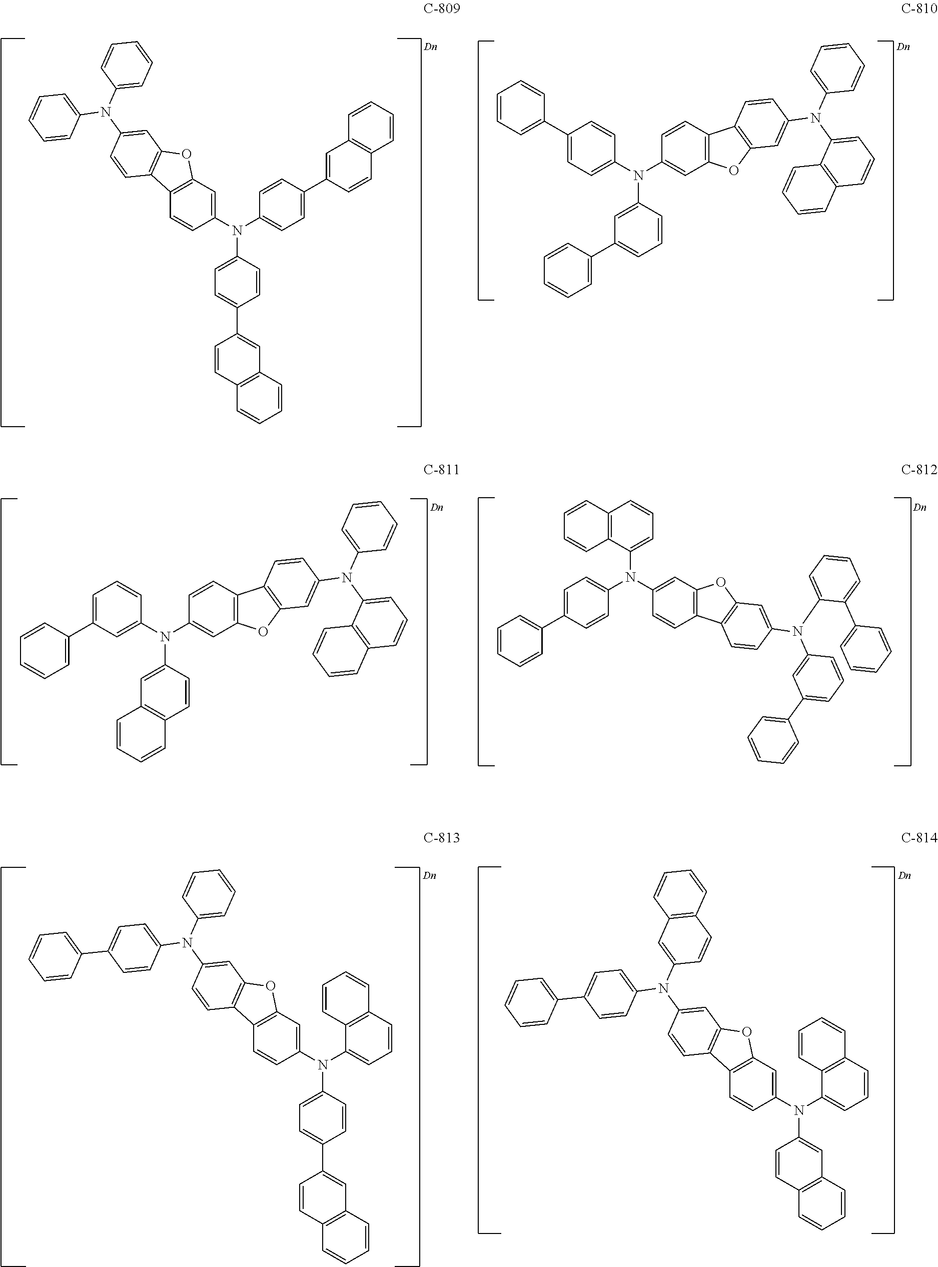 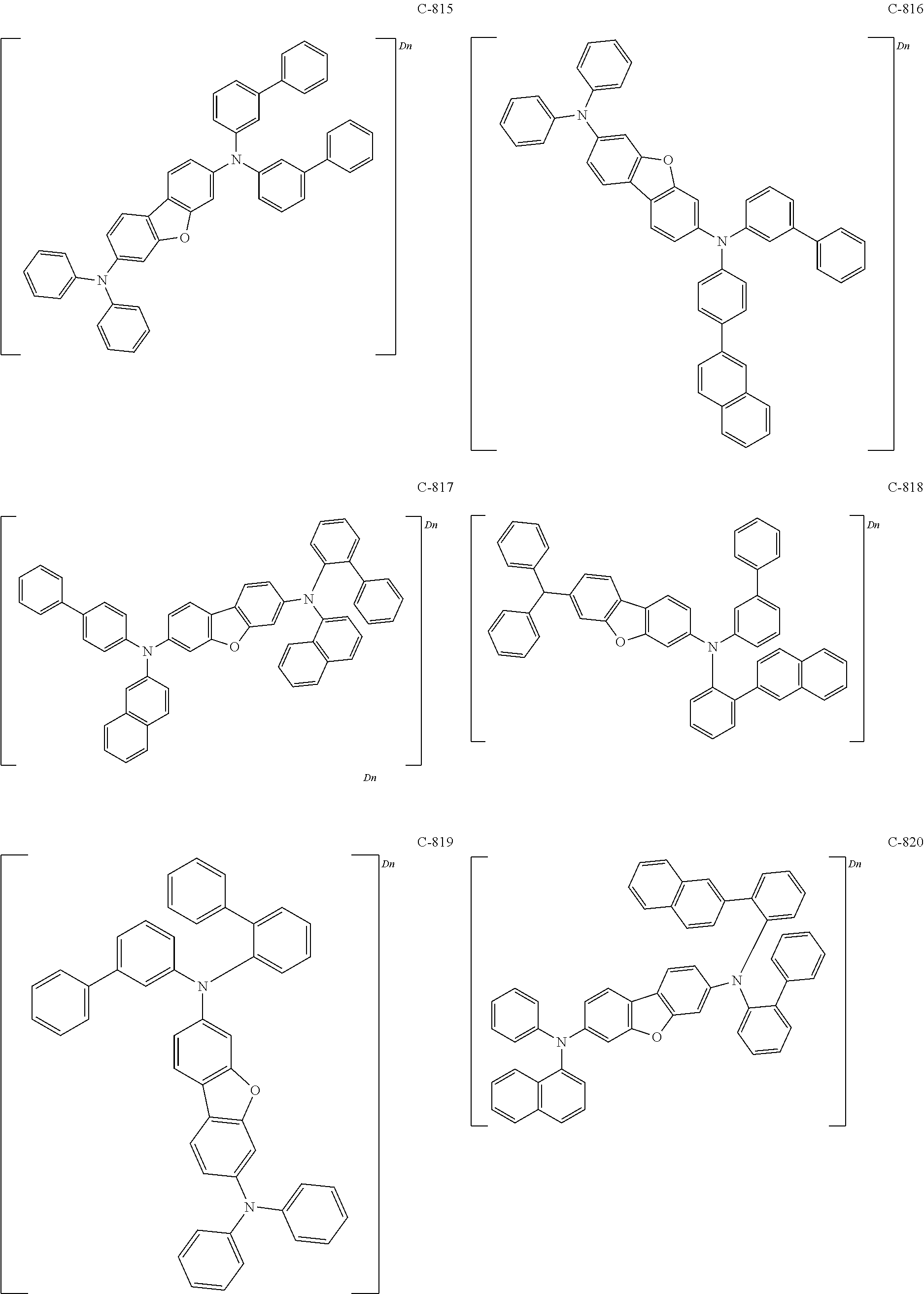 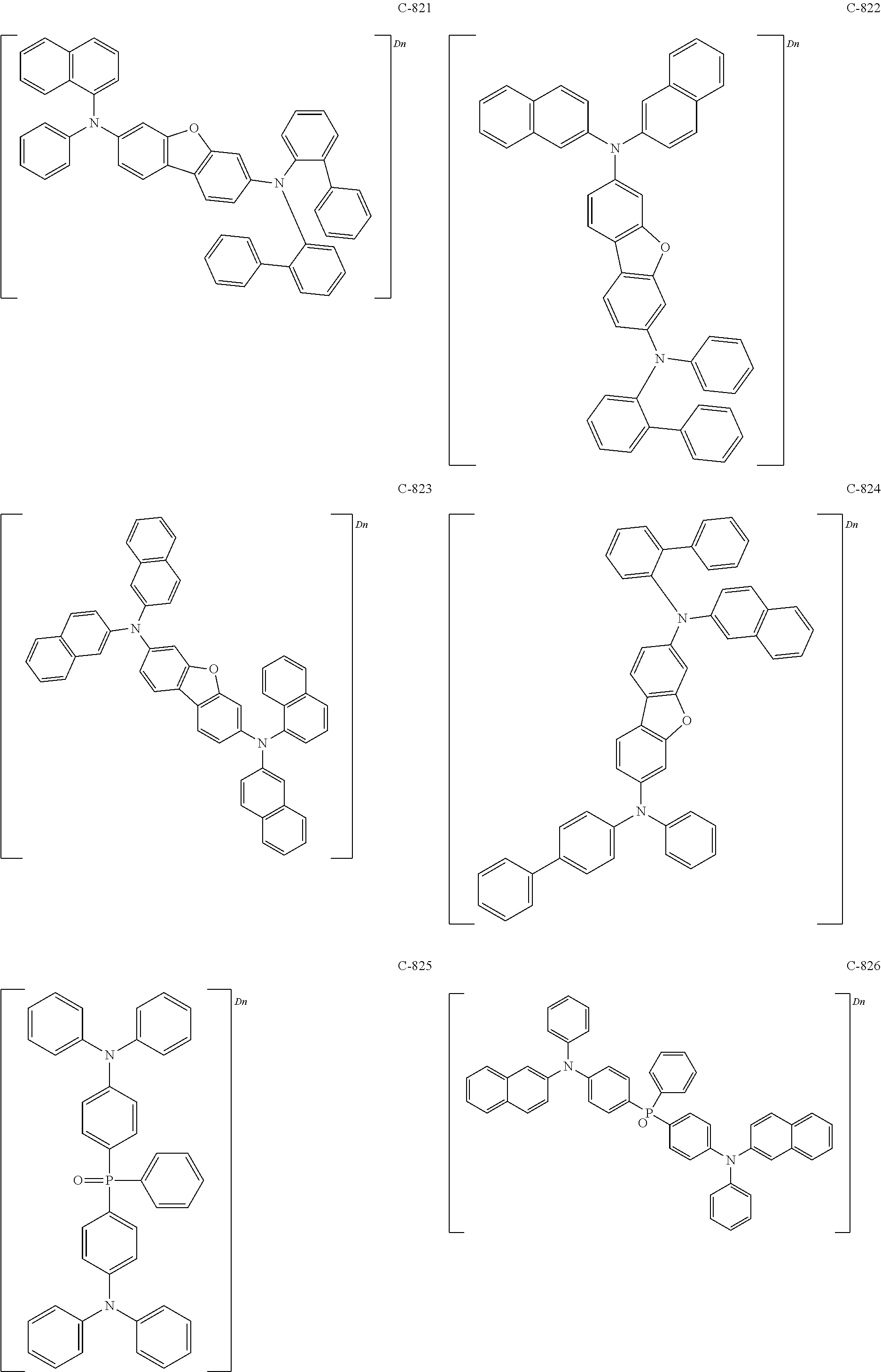 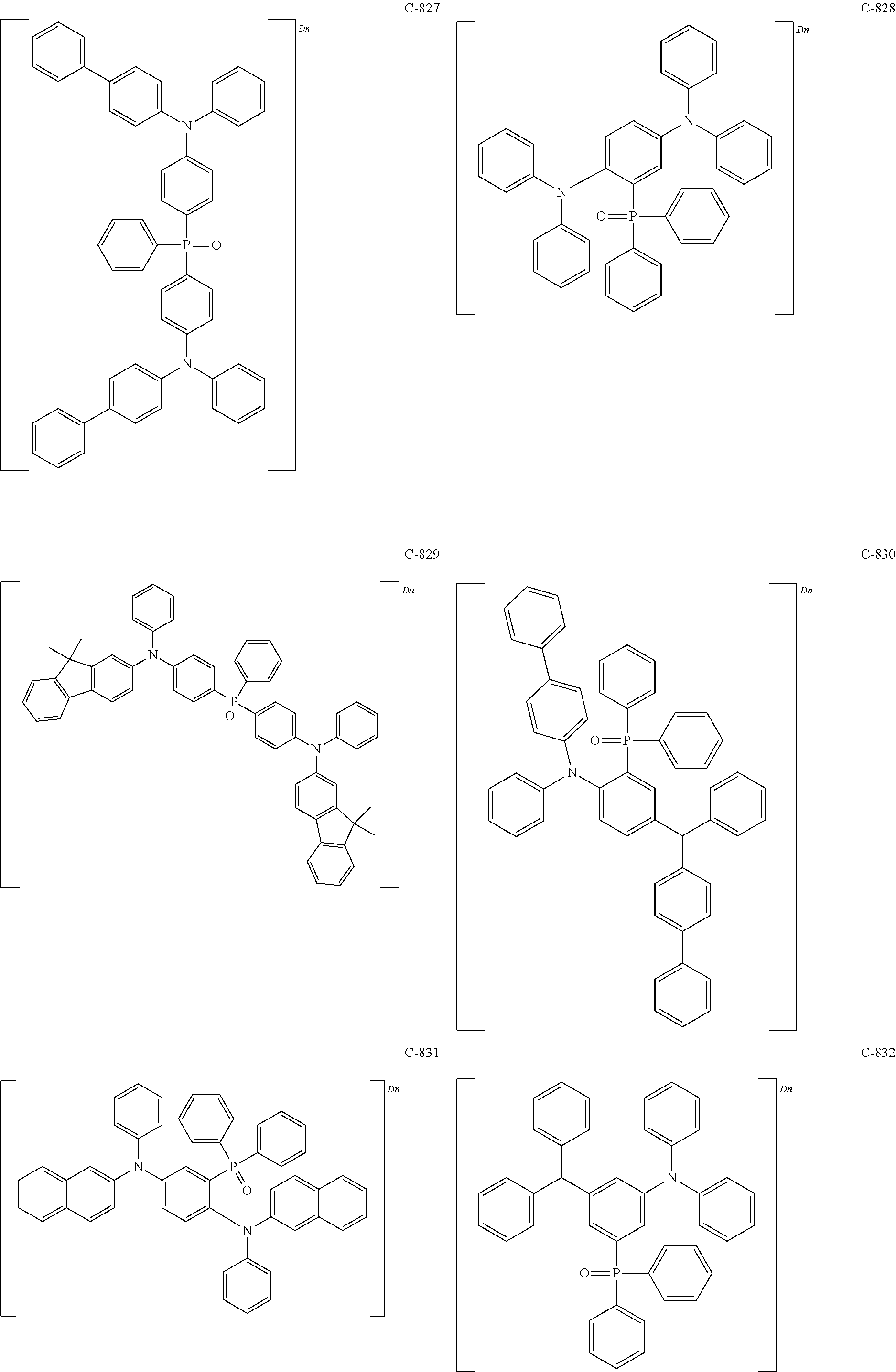 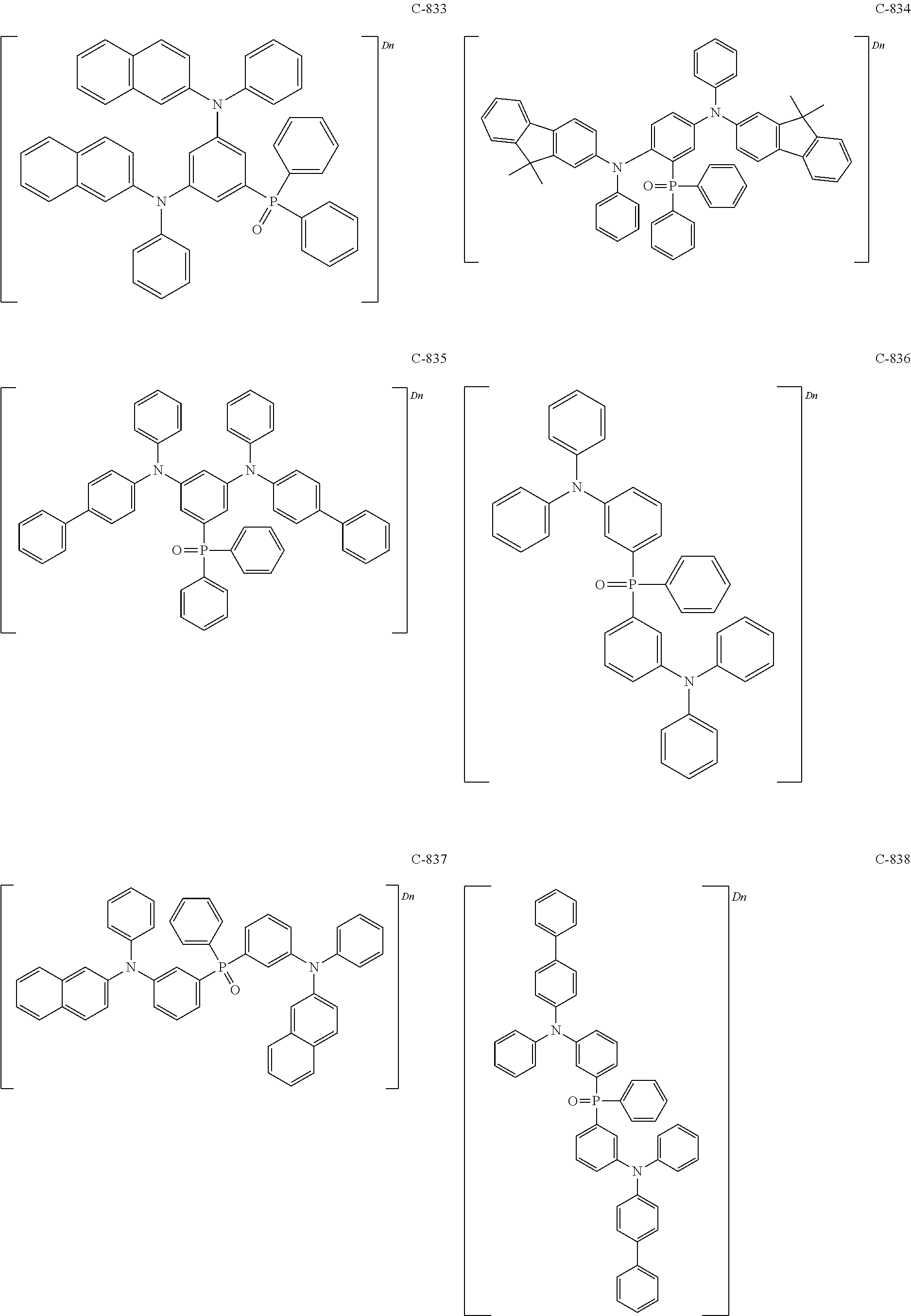 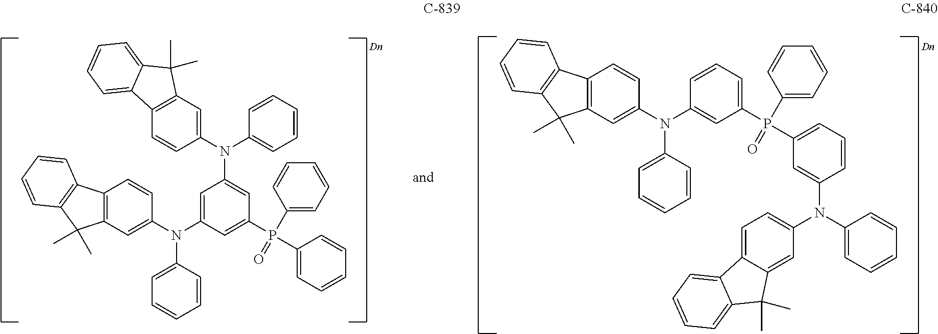
wherein in the compounds above, Dₙ means that n number of hydrogens are replaced with deuterium, and n represents an integer from 1 to the maximum number of hydrogens in the compound.

18. An organic electroluminescent device comprising the organic electroluminescent material according to claim 15.
